(19) United States
(12) Patent Application Publication
(10) Pub. No.: US 2023/0227856 A1
(43) Pub. Date: Jul. 20, 2023

(54) SELECTION BY ESSENTIAL-GENE KNOCK-IN

(71) Applicants: Editas Medicine, Inc., Cambridge, MA (US); BlueRock Therapeutics LP, Cambridge, MA (US)

(72) Inventors: John Anthony Zuris, Boston, MA (US); Carrie Marie Margulies, Waban, MA (US); Chew-Li Soh, Brooklyn, NY (US); Peter Tonge, Toronto (CA); Mark James Tomishima, Springfield, NJ (US); Conor Brian McAuliffe, Morristown, NJ (US); Claudio Monetti, Toronto (CA)

(21) Appl. No.: 17/923,358

(22) PCT Filed: May 4, 2021
(86) PCT No.: PCT/US2021/030744

§ 371 Date: Nov. 4, 2022

Related U.S. Application Data
(60) Provisional application No. 63/019,950, filed on May 4, 2020.

(51) Int. Cl. C12N 15/90; C12N 15/11; C12N 9/22; C12N 15/10; C12N 5/074; C12N 5/0783

(52) U.S. Cl. CPC C12N 15/907; C12N 15/11; C12N 9/22; C12N 15/1082; C12N 5/0696; C12N 5/0636; C12N 5/0646; C12N 2310/20; C12N 2800/80

(57) ABSTRACT

Strategies, systems, compositions, and methods for efficient production of knock-in cellular clones without reporter genes. An essential gene is targeted using a knock-in cassette that comprises an exogenous coding sequence for a gene product of interest (or “cargo sequence”) in frame with and downstream (3′) of an exogenous coding sequence or partial coding sequence of the essential gene. Undesired targeting events create a non-functional version of the essential gene, in essence a knock-out, which is “rescued” by correct integration of the knock-in cassette, which restores the essential gene coding region so that a functional gene product is produced and positions the cargo sequence in frame with and downstream of the essential gene coding sequence.

### CROSS REFERENCE TO RELATED APPLICATIONS

This application claims the benefit of U.S. Provisional Application No. 63/019,950, filed May 4, 2020, the contents of which is hereby incorporated in its entirety.

### BACKGROUND

One major problem with targeted integration strategies for the generation of genetically engineered cells is that successful targeted integration events can be rare, especially when using double-stranded DNA (dsDNA) as a template where knock-in efficiencies are often below 5%. There is therefore typically a requirement for a screening or selection strategy that enriches for cellular clones that harbor a successfully integrated allele or gene. Many selection strategies have been devised to identify correctly targeted clones, e.g., by co-integration of reporter genes that confer fluorescence, antibiotic resistance, etc. However, these selection strategies are time consuming, inefficient and not desirable for use in a therapeutic context. Indeed, even for a single targeted integration, it can be necessary to screen hundreds, sometimes thousands, of clones in order to identify a successfully targeted clone. In situations where multiple edits are desired it can be necessary to screen tens of thousands of clones or more.

### SUMMARY

The present disclosure provides strategies, systems, compositions, and methods for genetically engineering cells via targeted integration that do not require external selection markers, such as fluorescent or antibiotic resistance markers, while yielding a high frequency of correctly targeted clones. In general, the strategies, systems, compositions, and methods for genetically engineering cells via targeted integration provided herein feature a targeted break in an essential gene mediated by a nuclease, and integration of an exogenous knock-in cassette that, if inserted correctly, results in a functional variant of the essential gene and also includes an expression construct harboring a cargo sequence.

In one aspect, the disclosure features a method of editing the genome of a cell (e.g., a cell in a population of cells), the method comprising contacting the cell (or the population of cells) with: (i) a nuclease that causes a break within an endogenous coding sequence of an essential gene in the cell, wherein the essential gene encodes a gene product that is required for survival and/or proliferation of the cell, and (ii) a donor template that comprises a knock-in cassette comprising an exogenous coding sequence for a gene product of interest in frame with and downstream (3′) of an exogenous coding sequence or partial coding sequence of the essential gene, wherein the knock-in cassette is integrated into the genome of the cell by homology-directed repair (HDR) of the break, resulting in a genome-edited cell that expresses: (a) the gene product of interest, and (b) the gene product encoded by the essential gene that is required for survival and/or proliferation of the cell, or a functional variant thereof.

In some embodiments, following the contacting step, at least about 60%, at least about 65%, at least about 70%, at least about 75%, at least about 80%, at least about 85%, at least about 90%, at least about 95%, or more, of the viable cells of the population of cells are genome-edited cells, and/or about 40% or less, about 35% or less, about 30% or less, about 25% or less, about 20% or less, about 15% or less, about 10% or less, or about 5% or less, of the population of cells lacking an integrated knock-in cassette are viable cells. In some embodiments, following the contacting step, at least about 80% of the viable cells of the population of cells are genome-edited cells, and about 20% or less of the population of cells lacking an integrated knock-in cassette are viable cells. In some embodiments, following the contacting step, at least about 60% of the viable cells of the population of cells are genome-edited cells, and about 40% or less of the population of cells lacking an integrated knock-in cassette are viable cells. In some embodiments, following the contacting step, at least about 90% of the viable cells of the population of cells are genome-edited cells, and about 10% or less of the population of cells lacking an integrated knock-in cassette are viable cells. In some embodiments, following the contacting step, at least about 95% of the viable cells of the population of cells are genome-edited cells, and about 5% or less of the population of cells lacking an integrated knock-in cassette are viable cells.

In some embodiments, if the knock-in cassette is not integrated into the genome of the cell by homology-directed repair (HDR) in the correct position or orientation, the cell no longer expresses the gene product encoded by the essential gene, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the essential gene. In some embodiments, the break is located within the last exon of the essential gene. In some embodiments, the break is located within the penultimate exon of the essential gene.

In some embodiments, the nuclease is highly efficient, e.g., capable of editing at least about 60%, at least about 65%, at least about 75%, at least about 80%, at least about 85%, at least about 90%, at least about 95%, or more, of cells contacted with the nuclease. In some embodiments, the nuclease is capable of introducing indels (insertions or deletions) in at least about 60%, at least about 65%, at least about 75%, at least about 80%, at least about 85%, at least about 90%, at least about 95%, or more, of cells contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the cell (or the population of cells) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the nuclease is a CRISPR/Cas nuclease selected from Table 5. In some embodiments, the guide molecule comprises a targeting domain sequence that is complementary to a portion of the endogenous coding sequence of the essential gene. In some embodiments, the guide molecule comprises a targeting domain sequence that differs by no more than 3 nucleotides from a sequence that is complementary to a portion of the endogenous coding sequence of the essential gene. In some embodiments, the guide molecule specifically binds to the portion of the endogenous coding sequence of the essential gene. In some embodiments, the guide molecule does not bind to an endogenous coding sequence of another gene, e.g., a different essential gene. In some embodiments, the guide molecule binds to and mediates CRISPR/Cas cleavage at a location within the essential gene that is necessary for function (e.g., functional gene expression or protein function). In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the cell. In some embodiments, the donor template comprises a 3′ homology arm comprising a sequence homologous to a sequence located 3′ of the break in the genome of the cell. In some embodiments, the donor template comprises a 5′ homology arm comprising a sequence homologous to a sequence located 5′ of the break in the genome of the cell, and the donor template comprises a 3′ homology arm comprising a sequence homologous to a sequence located 3′ of the break in the genome of the cell.

In some embodiments, the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the essential gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the essential gene in the knock-in cassette encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, the exogenous coding sequence or partial coding sequence of the essential gene in the knock-in cassette is less than 100% identical to the corresponding endogenous coding sequence of the essential gene of the cell, e.g., less than 99%, less than 95%, less than 90%, less than 85%, or less than 80% identical to the corresponding endogenous coding sequence of the essential gene of the cell. In some embodiments, the exogenous coding sequence or partial coding sequence of the essential gene in the knock-in cassette is 80% to 99% identical to the corresponding endogenous coding sequence of the essential gene of the cell, e.g., 85% to 95% or 90% to 99% identical to the corresponding endogenous coding sequence of the essential gene of the cell. In some embodiments, the exogenous coding sequence or partial coding sequence of the essential gene in the knock-in cassette has been codon optimized relative to the corresponding endogenous coding sequence of the essential gene of the cell to remove a target site of the nuclease, to reduce the likelihood of homologous recombination after integration of the knock-in cassette into the genome of the cell, or to increase expression of the gene product of the essential gene and/or the gene product of interest after integration of the knock-in cassette into the genome of the cell.

In some embodiments, the nuclease is a Cas (e.g., Cas9 or Cas12a), the exogenous coding sequence or partial coding sequence of the essential gene in the knock-in cassette includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11. In some embodiments, the essential gene is a gene selected from Table 3, Table 4, or Table 17.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited cell comprises knock-in cassettes at one or both alleles of the essential gene. In some embodiments, the genome-edited cell expresses (a) the first and second gene products of interest, and (b) the gene product encoded by the essential gene that is required for survival and/or proliferation of the cell, or a functional variant thereof. In some embodiments, the genome-edited cell expresses (a) the first and second gene products of interest from the same allele of an essential gene, and (b) the gene product encoded by the essential gene that is required for survival and/or proliferation of the cell, or a functional variant thereof. In some embodiments, the genome-edited cell expresses (a) the first and second gene products of interest from different alleles of the essential gene, and (b) the gene product encoded by the essential gene that is required for survival and/or proliferation of the cell, or a functional variant thereof.

In some embodiments, the method comprises contacting the cell (or the population of cells) with a first a donor template that comprises a first knock-in cassette comprising a first exogenous coding sequence for a first gene product of interest in frame with and downstream (3′) of an exogenous coding sequence or partial coding sequence of the essential gene, and with a second donor template that comprises a second knock-in cassette comprising a second exogenous coding sequence for a second gene product of interest in frame with and downstream (3′) of an exogenous coding sequence or partial coding sequence of the essential gene. In some embodiments, the genome-edited cell comprises the first knock-in cassette at a first allele of the essential gene and the second knock-in cassette at the second allele of the essential gene. In some embodiments, the genome-edited cell expresses (a) the first and second gene products of interest, and (b) the gene product encoded by the essential gene that is required for survival and/or proliferation of the cell, or a functional variant thereof.

In some embodiments, the method comprises contacting the cell (or the population of cells) with a first a donor template that comprises a first knock-in cassette comprising a first exogenous coding sequence for a first gene product of interest in frame with and downstream (3′) of an exogenous coding sequence or partial coding sequence of a first essential gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, the genome-edited cell comprises the first knock-in cassette at one or both alleles of the first essential gene and the second knock-in cassette at one or both alleles of the second essential gene. In some embodiments, the genome-edited cell expresses (a) the first and second gene products of interest, and (b) the gene products encoded by the first and second essential genes required for survival and/or proliferation of the cell, or a functional variant thereof.

In another aspect, the disclosure features a genetically modified cell comprising a genome with an exogenous coding sequence for a gene product of interest in frame with and downstream (3′) of a coding sequence of an essential gene, wherein the essential gene encodes a gene product that is required for survival and/or proliferation of the cell, and wherein at least part of the coding sequence of the essential gene comprises an exogenous coding sequence.

In some embodiments, the exogenous coding sequence of the essential gene comprises about 2000, 1500, 1000, 750, 500, 400, 300, 200, 100, or 50 base pairs of the coding sequence of the essential gene.

In some embodiments, the exogenous coding sequence of the essential gene encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, the exogenous coding sequence of the essential gene is less than 100% identical to the corresponding endogenous coding sequence of the essential gene of the cell. In some embodiments, the exogenous coding sequence of the essential gene has been codon optimized relative to the corresponding endogenous coding sequence of the essential gene of the cell to remove a target site of a nuclease, e.g., a Cas. In some embodiments, the nuclease is a Cas (e.g., Cas9 or Cas12a), the exogenous coding sequence of the essential gene includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11.

In some embodiments, the cell's genome comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the cell's genome comprises an IRES or 2A element located between the coding sequence of the essential gene and the exogenous coding sequence for the gene product of interest.

In some embodiments, the cell's genome comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the cell's genome does not comprise a reporter gene, e.g., a fluorescent reporter gene or an antibiotic resistance gene.

In another aspect, the disclosure features an engineered cell comprising a genomic modification, wherein the genomic modification comprises an insertion of an exogenous knock-in cassette within an endogenous coding sequence of an essential gene in the cell's genome, wherein the essential gene encodes a gene product that is required for survival and/or proliferation of the cell, wherein the knock-in cassette comprises an exogenous coding sequence for a gene product of interest in frame with and downstream (3′) of an exogenous coding sequence or partial coding sequence encoding the gene product of the essential gene, or a functional variant thereof, and wherein the cell expresses the gene product of interest and the gene product encoded by the essential gene that is required for survival and/or proliferation of the cell, or a functional variant thereof, optionally wherein the gene product of interest and the gene product encoded by the essential gene are expressed from the endogenous promoter of the essential gene.

In some embodiments, the exogenous coding sequence or partial coding sequence encoding the gene product of the essential gene comprises about 2000, 1500, 1000, 750, 500, 400, 300, 200, 100, or 50 base pairs of the coding sequence of the essential gene.

In some embodiments, wherein the exogenous coding sequence or partial coding sequence encoding the gene product of the essential gene encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, exogenous coding sequence or partial coding sequence encoding the gene product of the essential gene is less than 100% identical to the corresponding endogenous coding sequence of the essential gene of the cell. In some embodiments, the exogenous coding sequence or partial coding sequence encoding the gene product of the essential gene has been codon optimized relative to the corresponding endogenous coding sequence of the essential gene of the cell to remove a target site of a nuclease, e.g., a Cas. In some embodiments, the nuclease is a Cas (e.g., Cas9 or Cas12a), the exogenous coding sequence or partial coding sequence encoding the gene product of the essential gene includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11.

In some embodiments, the exit's genome comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the cell's genome comprises an IRES or 2A element located between the coding sequence of the essential gene and the exogenous coding sequence for the gene product of interest.

In some embodiments, the cell's genome comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the cell's genome does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited cell comprises knock-in cassettes at one or both alleles of the essential gene. In some embodiments, the genome-edited cell expresses (a) the first and second gene products of interest, and (b) the gene product encoded by the essential gene that is required for survival and/or proliferation of the cell, or a functional variant thereof.

In some embodiments, the engineered cell comprises a first knock-in cassette comprising a first exogenous coding sequence for a first gene product of interest in frame with and downstream (3′) of an exogenous coding sequence or partial coding sequence of the essential gene, and with a second donor template that comprises a second knock-in cassette comprising a second exogenous coding sequence for a second gene product of interest in frame with and downstream (3′) of an exogenous coding sequence or partial coding sequence of the essential gene. In some embodiments, the engineered cell comprises the first knock-in cassette and the second knock-in cassette at a first allele of the essential gene, optionally wherein the engineered cell also comprises the first knock-in cassette and the second knock-in cassette at a second allele of the essential gene. In some embodiments, the engineered cell comprises the first knock-in cassette at a first allele of the essential gene and the second knock-in cassette at the second allele of the essential gene. In some embodiments, the engineered cell expresses (a) the first and second gene products of interest, and (b) the gene product encoded by the essential gene that is required for survival and/or proliferation of the cell, or a functional variant thereof.

In some embodiments, the engineered cell comprises a first knock-in cassette comprising a first exogenous coding sequence for a first gene product of interest in frame with and downstream (3′) of an exogenous coding sequence or partial coding sequence of a first essential gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, the engineered cell comprises the first knock-in cassette at one or both alleles of the first essential gene and the second knock-in cassette at one or both alleles of the second essential gene. In some embodiments, the genome-edited cell expresses (a) the first and second gene products of interest, and (b) the gene products encoded by the first and second essential genes required for survival and/or proliferation of the cell, or a functional variant thereof.

In another aspect, the disclosure features any of the cells described herein for use as a medicament and/or for use in the treatment of a disease, disorder or condition, e.g., a disease, disorder or condition described herein, e.g., a cancer, e.g., a cancer described herein.

In another aspect, the disclosure features a cell, or a population of cells, produced by any of the methods described herein, or progeny thereof.

In another aspect, the disclosure features a system for editing the genome of a cell (or a cell in a population of cells), the system comprising the cell (or the population of cells), a nuclease that causes a break within an endogenous coding sequence of an essential gene of the cell, wherein the essential gene encodes a gene product that is required for survival and/or proliferation of the cell, and a donor template that comprises a knock-in cassette comprising an exogenous coding sequence for a gene product of interest in frame with and downstream (3′) of an exogenous coding sequence or partial coding sequence of the essential gene.

In some embodiments, after contacting the population of cells with the nuclease and the donor template, at least about 60%, at least about 65%, at least about 70%, at least about 75%, at least about 80%, at least about 85%, at least about 90%, at least about 95%, or more, of the viable cells of the population of cells are genome-edited cells, and/or about 40% or less, about 35% or less, about 30% or less, about 25% or less, about 20% or less, about 15% or less, about 10% or less, or about 5% or less, of the population of cells lacking an integrated knock-in cassette are viable cells. In some embodiments, after contacting the population of cells with the nuclease and the donor template, at least about 80% of the viable cells of the population of cells are genome-edited cells, and about 20% or less of the population of cells lacking an integrated knock-in cassette are viable cells. In some embodiments, after contacting the population of cells with the nuclease and the donor template, at least about 60% of the viable cells of the population of cells are genome-edited cells, and about 40% or less of the population of cells lacking an integrated knock-in cassette are viable cells. In some embodiments, after contacting the population of cells with the nuclease and the donor template, at least about 90% of the viable cells of the population of cells are genome-edited cells, and about 10% or less of the population of cells lacking an integrated knock-in cassette are viable cells. In some embodiments, after contacting the population of cells with the nuclease and the donor template, at least about 95% of the viable cells of the population of cells are genome-edited cells, and about 5% or less of the population of cells lacking an integrated knock-in cassette are viable cells.

In some embodiments, after contacting the cell or population of cells with the nuclease and the donor template, if the knock-in cassette is not integrated into the genome of the cell by homology-directed repair (HDR) in the correct position or orientation, the cell no longer expresses the gene product encoded by the essential gene, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the essential gene. In some embodiments, the break is located within the last exon of the essential gene.

In some embodiments, the nuclease is highly efficient, e.g., capable of editing at least about 60%, at least about 65%, at least about 75%, at least about 80%, at least about 85%, at least about 90%, at least about 95%, or more, of cells contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the cell (or the population of cells) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the essential gene. In some embodiments, the guide molecule comprises a targeting domain sequence that differs by no more than 3 nucleotides from a sequence that is complementary to a portion of the endogenous coding sequence of the essential gene. In some embodiments, the guide molecule specifically binds to the portion of the endogenous coding sequence of the essential gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the cell. In some embodiments, the donor template comprises a 3′ homology arm comprising a sequence homologous to a sequence located 3′ of the break in the genome of the cell. In some embodiments, the donor template comprises a 5′ homology arm comprising a sequence homologous to a sequence located 5′ of the break in the genome of the cell, and the donor template comprises a 3′ homology arm comprising a sequence homologous to a sequence located 3′ of the break in the genome of the cell.

In some embodiments, the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the essential gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the essential gene in the knock-in cassette encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, the exogenous coding sequence or partial coding sequence of the essential gene in the knock-in cassette is less than 100% identical to the corresponding endogenous coding sequence of the essential gene of the cell. In some embodiments, the exogenous coding sequence or partial coding sequence of the essential gene in the knock-in cassette has been codon optimized relative to the corresponding endogenous coding sequence of the essential gene of the cell to remove a target site of the nuclease, to reduce the likelihood of homologous recombination after integration of the knock-in cassette into the genome of the cell, or to increase expression of the gene product of the essential gene and/or the gene product of interest after integration of the knock-in cassette into the genome of the cell.

In some embodiments, the nuclease is a Cas (e.g., Cas9 or Cas12a), the exogenous coding sequence or partial coding sequence of the essential gene in the knock-in cassette includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, after contacting the population of cells with the nuclease and the donor template, the genome-edited cell comprises knock-in cassettes at one or both alleles of the essential gene. In some embodiments, the genome-edited cell expresses (a) the first and second gene products of interest, and (b) the gene product encoded by the essential gene that is required for survival and/or proliferation of the cell, or a functional variant thereof.

In some embodiments, the system comprises a first a donor template that comprises a first knock-in cassette comprising a first exogenous coding sequence for a first gene product of interest in frame with and downstream (3′) of an exogenous coding sequence or partial coding sequence of the essential gene, and with a second donor template that comprises a second knock-in cassette comprising a second exogenous coding sequence for a second gene product of interest in frame with and downstream (3′) of an exogenous coding sequence or partial coding sequence of the essential gene. In some embodiments, after contacting the population of cells with the nuclease and the donor templates, the genome-edited cell comprises the first knock-in cassette at a first allele of the essential gene and the second knock-in cassette at the second allele of the essential gene. In some embodiments, the genome-edited cell expresses (a) the first and second gene products of interest, and (b) the gene product encoded by the essential gene that is required for survival and/or proliferation of the cell, or a functional variant thereof.

In some embodiments, the system comprises a first a donor template that comprises a first knock-in cassette comprising a first exogenous coding sequence for a first gene product of interest in frame with and downstream (3′) of an exogenous coding sequence or partial coding sequence of a first essential gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, after contacting the population of cells with the nuclease and the donor templates, the genome-edited cell comprises the first knock-in cassette at one or both alleles of the first essential gene and the second knock-in cassette at one or both alleles of the second essential gene. In some embodiments, the genome-edited cell expresses (a) the first and second gene products of interest, and (b) the gene products encoded by the first and second essential genes required for survival and/or proliferation of the cell, or a functional variant thereof.

In another aspect, the disclosure features a donor template comprising a knock-in cassette with an exogenous coding sequence for a gene product of interest in frame with and downstream (3′) of an exogenous coding sequence or partial coding sequence of an essential gene, wherein the essential gene encodes a gene product that is required for survival and/or proliferation of the cell.

In some embodiments, the donor template is for use in editing the genome of a cell by homology-directed repair (HDR).

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of a target site in the genome of the cell. In some embodiments, the donor template comprises a 3′ homology arm comprising a sequence homologous to a sequence located 3′ of a target site in the genome of the cell. In some embodiments, the donor template comprises a 5′ homology arm comprising a sequence homologous to a sequence located 5′ of a target site in the genome of the cell, and the donor template comprises a 3′ homology arm comprising a sequence homologous to a sequence located 3′ of a target site in the genome of the cell.

In some embodiments, the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the essential gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the essential gene in the knock-in cassette encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene.

In some embodiments, the exogenous coding sequence or partial coding sequence of the essential gene in the knock-in cassette is less than 100% identical to the corresponding endogenous coding sequence of the essential gene of the cell. In some embodiments, the exogenous coding sequence or partial coding sequence of the essential gene in the knock-in cassette has been codon optimized relative to the corresponding endogenous coding sequence of the essential gene of the cell to remove a target site of the nuclease, to reduce the likelihood of homologous recombination after integration of the knock-in cassette into the genome of the cell, or to increase expression of the gene product of the essential gene and/or the gene product of interest after integration of the knock-in cassette into the genome of the cell.

In some embodiments, the nuclease is a Cas (e.g., Cas9 or Cas12a), the exogenous coding sequence or partial coding sequence of the essential gene in the knock-in cassette includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11.

In some embodiments, the donor template does not comprise a reporter gene, e.g., a fluorescent reporter gene or an antibiotic resistance gene.

In one aspect, the disclosure features a method of producing a population of modified cells, the method comprising contacting cells with: (i) a nuclease that causes a break within an endogenous coding sequence of an essential gene in a plurality of the cells, wherein the essential gene encodes a gene product that is required for survival and/or proliferation of the cells, and (ii) a donor template that comprises a knock-in cassette comprising an exogenous coding sequence for a gene product of interest in frame with and downstream (3′) of an exogenous coding sequence or partial coding sequence of the essential gene, wherein the knock-in cassette is integrated into the genome of a plurality of the cells by homology-directed repair (HDR) of the break, resulting in genome-edited cells that expresses: (a) the gene product of interest, and (b) the gene product encoded by the essential gene that is required for survival and/or proliferation of the plurality of cells, or a functional variant thereof, and wherein following the contacting step, at least about 60%, at least about 65%, at least about 70%, at least about 75%, at least about 80%, at least about 85%, at least about 90%, at least about 95%, or more, of the viable cells are genome-edited cells, and/or about 40% or less, about 35% or less, about 30% or less, about 25% or less, about 20% or less, about 15% or less, about 10% or less, or about 5% or less, of the cells lacking an integrated knock-in cassette are viable cells, thereby producing a population of modified cells. In some embodiments, following the contacting step, at least about 80% of the viable cells are genome-edited cells, and about 20% or less of the cells lacking an integrated knock-in cassette are viable cells. In some embodiments, following the contacting step, at least about 60% of the viable cells are genome-edited cells, and about 40% or less of the cells lacking an integrated knock-in cassette are viable cells. In some embodiments, following the contacting step, at least about 90% of the viable cells are genome-edited cells, and about 10% or less of the cells lacking an integrated knock-in cassette are viable cells. In some embodiments, following the contacting step, at least about 95% of the viable cells are genome-edited cells, and about 5% or less of cells lacking an integrated knock-in cassette are viable cells.

In some embodiments, if the knock-in cassette is not integrated into the genome of the cell by homology-directed repair (HDR) in the correct position or orientation, the cell no longer expresses the gene product encoded by the essential gene, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the essential gene. In some embodiments, the break is located within the last exon of the essential gene.

In some embodiments, the nuclease is highly efficient, e.g., capable of editing at least about 60%, at least about 65%, at least about 75%, at least about 80%, at least about 85%, at least about 90%, at least about 95%, or more, of cells contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the cell (or the population of cells) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the essential gene. In some embodiments, the guide molecule comprises a targeting domain sequence that differs by no more than 3 nucleotides from a sequence that is complementary to a portion of the endogenous coding sequence of the essential gene. In some embodiments, the guide molecule specifically binds to the portion of the endogenous coding sequence of the essential gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the cell. In some embodiments, the donor template comprises a 3′ homology arm comprising a sequence homologous to a sequence located 3′ of the break in the genome of the cell. In some embodiments, the donor template comprises a 5′ homology arm comprising a sequence homologous to a sequence located 5′ of the break in the genome of the cell, and the donor template comprises a 3′ homology arm comprising a sequence homologous to a sequence located 3′ of the break in the genome of the cell.

In some embodiments, the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the essential gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the essential gene in the knock-in cassette encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, the exogenous coding sequence or partial coding sequence of the essential gene in the knock-in cassette is less than 100% identical to the corresponding endogenous coding sequence of the essential gene of the cell. In some embodiments, the exogenous coding sequence or partial coding sequence of the essential gene in the knock-in cassette has been codon optimized relative to the corresponding endogenous coding sequence of the essential gene of the cell to remove a target site of the nuclease, to reduce the likelihood of homologous recombination after integration of the knock-in cassette into the genome of the cell, or to increase expression of the gene product of the essential gene and/or the gene product of interest after integration of the knock-in cassette into the genome of the cell.

In some embodiments, the nuclease is a Cas (e.g., Cas9 or Cas12a), the exogenous coding sequence or partial coding sequence of the essential gene in the knock-in cassette includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited cells comprise knock-in cassettes at one or both alleles of the essential gene. In some embodiments, the genome-edited cells expresses (a) the first and second gene products of interest, and (b) the gene product encoded by the essential gene that is required for survival and/or proliferation of the cells, or a functional variant thereof.

In some embodiments, the method comprises contacting the cells (or the population of cells) with a first a donor template that comprises a first knock-in cassette comprising a first exogenous coding sequence for a first gene product of interest in frame with and downstream (3′) of an exogenous coding sequence or partial coding sequence of the essential gene, and with a second donor template that comprises a second knock-in cassette comprising a second exogenous coding sequence for a second gene product of interest in frame with and downstream (3′) of an exogenous coding sequence or partial coding sequence of the essential gene. In some embodiments, the genome-edited cells comprise the first knock-in cassette at a first allele of the essential gene and the second knock-in cassette at the second allele of the essential gene. In some embodiments, the genome-edited cells expresses (a) the first and second gene products of interest, and (b) the gene product encoded by the essential gene that is required for survival and/or proliferation of the cells, or a functional variant thereof.

In some embodiments, the method comprises contacting the cells (or the population of cells) with a first a donor template that comprises a first knock-in cassette comprising a first exogenous coding sequence for a first gene product of interest in frame with and downstream (3′) of an exogenous coding sequence or partial coding sequence of a first essential gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, the genome-edited cells comprise the first knock-in cassette at one or both alleles of the first essential gene and the second knock-in cassette at one or both alleles of the second essential gene. In some embodiments, the genome-edited cells expresses (a) the first and second gene products of interest, and (b) the gene products encoded by the first and second essential genes required for survival and/or proliferation of the cells, or a functional variant thereof.

In another aspect, the disclosure features a method of selecting and/or identifying a cell comprising a knock-in of a gene product of interest within an endogenous coding sequence of an essential gene in the cell, the method comprising contacting a population of cells with: (i) a nuclease that causes a break within an endogenous coding sequence of an essential gene in a plurality of the cells, wherein the essential gene encodes a gene product that is required for survival and/or proliferation of the cells, and (ii) a donor template that comprises a knock-in cassette comprising an exogenous coding sequence for a gene product of interest in frame with and downstream (3′) of an exogenous coding sequence or partial coding sequence of the essential gene, wherein the knock-in cassette is integrated into the genome of a plurality of the cells by homology-directed repair (HDR) of the break, and identifying a genome-edited cell within the population of cells that expresses: (a) the gene product of interest, and (b) the gene product encoded by the essential gene that is required for survival and/or proliferation of the cell, or a functional variant thereof.

In some embodiments, following the contacting step, at least about 60%, at least about 65%, at least about 70%, at least about 75%, at least about 80%, at least about 85%, at least about 90%, at least about 95%, or more, of the viable cells of the population of cells are genome-edited cells, and/or about 40% or less, about 35% or less, about 30% or less, about 25% or less, about 20% or less, about 15% or less, about 10% or less, or about 5% or less, of the population of cells lacking an integrated knock-in cassette are viable cells. In some embodiments, following the contacting step, at least about 80% of the viable cells of the population of cells are genome-edited cells, and about 20% or less of the population of cells lacking an integrated knock-in cassette are viable cells. In some embodiments, following the contacting step, at least about 60% of the viable cells of the population of cells are genome-edited cells, and about 40% or less of the population of cells lacking an integrated knock-in cassette are viable cells. In some embodiments, following the contacting step, at least about 90% of the viable cells of the population of cells are genome-edited cells, and about 10% or less of the population of cells lacking an integrated knock-in cassette are viable cells. In some embodiments, following the contacting step, at least about 95% of the viable cells of the population of cells are genome-edited cells, and about 5% or less of the population of cells lacking an integrated knock-in cassette are viable cells.

In some embodiments, if the knock-in cassette is not integrated into the genome of the cell by homology-directed repair (HDR) in the correct position or orientation, the cell no longer expresses the gene product encoded by the essential gene, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the essential gene. In some embodiments, the break is located within the last exon of the essential gene.

In some embodiments, the nuclease is highly efficient, e.g., capable of editing at least about 60%, at least about 65%, at least about 75%, at least about 80%, at least about 85%, at least about 90%, at least about 95%, or more, of cells contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the cell (or the population of cells) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the essential gene. In some embodiments, the guide molecule comprises a targeting domain sequence that differs by no more than 3 nucleotides from a sequence that is complementary to a portion of the endogenous coding sequence of the essential gene. In some embodiments, the guide molecule specifically binds to the portion of the endogenous coding sequence of the essential gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the cell. In some embodiments, the donor template comprises a 3′ homology arm comprising a sequence homologous to a sequence located 3′ of the break in the genome of the cell. In some embodiments, the donor template comprises a 5′ homology arm comprising a sequence homologous to a sequence located 5′ of the break in the genome of the cell, and the donor template comprises a 3′ homology arm comprising a sequence homologous to a sequence located 3′ of the break in the genome of the cell.

In some embodiments, the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the essential gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the essential gene in the knock-in cassette encodes a C-terminal fragment of a protein encoded by the essential gene. In some embodiments, the C-terminal fragment is less than about 500, 250, 150, 125, 100, 75, 50, 25, 20, 15 or 10 amino acids in length. In some embodiments, the C-terminal fragment includes an amino acid sequence that is encoded by a region of the endogenous coding sequence of the essential gene that spans the break.

In some embodiments, the exogenous coding sequence or partial coding sequence of the essential gene in the knock-in cassette is less than 100% identical to the corresponding endogenous coding sequence of the essential gene of the cell. In some embodiments, the exogenous coding sequence or partial coding sequence of the essential gene in the knock-in cassette has been codon optimized relative to the corresponding endogenous coding sequence of the essential gene of the cell to remove a target site of the nuclease, to reduce the likelihood of homologous recombination after integration of the knock-in cassette into the genome of the cell, or to increase expression of the gene product of the essential gene and/or the gene product of interest after integration of the knock-in cassette into the genome of the cell.

In some embodiments, the nuclease is a Cas (e.g., Cas9 or Cas12a), the exogenous coding sequence or partial coding sequence of the essential gene in the knock-in cassette includes at least one PAM site for the Cas, and the at least one PAM site (or all PAM sites) has been codon optimized or saturated with silent and/or missense mutations.

In some embodiments, the essential gene is GAPDH, TBP, E2F4, G6PD, or KIF11.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited cell comprises knock-in cassettes at one or both alleles of the essential gene. In some embodiments, the genome-edited cell expresses (a) the first and second gene products of interest, and (b) the gene product encoded by the essential gene that is required for survival and/or proliferation of the cell, or a functional variant thereof.

In some embodiments, the method comprises contacting the population of cells with a first a donor template that comprises a first knock-in cassette comprising a first exogenous coding sequence for a first gene product of interest in frame with and downstream (3′) of an exogenous coding sequence or partial coding sequence of the essential gene, and with a second donor template that comprises a second knock-in cassette comprising a second exogenous coding sequence for a second gene product of interest in frame with and downstream (3′) of an exogenous coding sequence or partial coding sequence of the essential gene. In some embodiments, the genome-edited cells comprises the first knock-in cassette at a first allele of the essential gene and the second knock-in cassette at the second allele of the essential gene. In some embodiments, the genome-edited cells expresses (a) the first and second gene products of interest, and (b) the gene product encoded by the essential gene that is required for survival and/or proliferation of the cell, or a functional variant thereof.

In some embodiments, the method comprises contacting the population of cells with a first a donor template that comprises a first knock-in cassette comprising a first exogenous coding sequence for a first gene product of interest in frame with and downstream (3′) of an exogenous coding sequence or partial coding sequence of a first essential gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, the genome-edited cells comprises the first knock-in cassette at one or both alleles of the first essential gene and the second knock-in cassette at one or both alleles of the second essential gene. In some embodiments, the genome-edited cell expresses (a) the first and second gene products of interest, and (b) the gene products encoded by the first and second essential genes required for survival and/or proliferation of the cell, or a functional variant thereof.

In another aspect, the disclosure features a method of editing the genome of an induced pluripotent stem cell (iPSC) (e.g., an iPSC in a population of iPSCs), the method comprising contacting the iPSC (or the population of iPSCs) with: (i) a nuclease that causes a break within an endogenous coding sequence of a glyceraldehyde 3-phosphate dehydrogenase (GAPDH) gene in the iPSC, and (ii) a donor template that comprises a knock-in cassette comprising an exogenous coding sequence for a gene product of interest in frame with and downstream (3′) of an exogenous coding sequence or partial coding sequence of the GAPDH gene, wherein the knock-in cassette is integrated into the genome of the iPSC by homology-directed repair (HDR) of the break, resulting in a genome-edited iPSC that expresses: (a) the gene product of interest, and (b) GAPDH, or a functional variant thereof.

In some embodiments, following the contacting step, at least about 60%, at least about 65%, at least about 70%, at least about 75%, at least about 80%, at least about 85%, at least about 90%, at least about 95%, or more, of the viable iPSCs of the population of iPSCs are genome-edited iPSCs, and/or about 40% or less, about 35% or less, about 30% or less, about 25% or less, about 20% or less, about 15% or less, about 10% or less, or about 5% or less, of the population of iPSCs lacking an integrated knock-in cassette are viable iPSCs. In some embodiments, following the contacting step, at least about 80% of the viable iPSCs of the population of iPSCs are genome-edited iPSCs, and about 20% or less of the population of iPSCs lacking an integrated knock-in cassette are viable iPSCs. In some embodiments, following the contacting step, at least about 60% of the viable iPSCs of the population of iPSCs are genome-edited iPSCs, and about 40% or less of the population of iPSCs lacking an integrated knock-in cassette are viable iPSCs. In some embodiments, following the contacting step, at least about 90% of the viable iPSCs of the population of iPSCs are genome-edited iPSCs, and about 10% or less of the population of iPSCs lacking an integrated knock-in cassette are viable iPSCs. In some embodiments, following the contacting step, at least about 95% of the viable iPSCs of the population of iPSCs are genome-edited iPSCs, and about 5% or less of the population of iPSCs lacking an integrated knock-in cassette are viable iPSCs.

In some embodiments, if the knock-in cassette is not integrated into the genome of the iPSCs by homology-directed repair (HDR) in the correct position or orientation, the iPSCs no longer expresses GAPDH, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of a protein encoded by the GAPDH gene. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the method comprises contacting the iPSC (or the population of iPSCs) with a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the genome-edited iPSC comprises the first knock-in cassette at a first allele of the GAPDH gene and the second knock-in cassette at the second allele of the GAPDH gene. In some embodiments, the genome-edited iPSC expresses (a) the first and second gene products of interest, and (b) GAPDH, or a functional variant thereof.

In another aspect, the disclosure features a genetically modified iPSC comprising a genome with an exogenous coding sequence for a gene product of interest in frame with and downstream (3′) of a coding sequence of a GAPDH gene, and wherein at least part of the coding sequence of the GAPDH gene comprises an exogenous coding sequence.

In some embodiments, the exogenous coding sequence of the GAPDH gene comprises about 2000, 1500, 1000, 750, 500, 400, 300, 200, 100, or 50 base pairs of the coding sequence of the GAPDH gene. In some embodiments, the exogenous coding sequence of the GAPDH gene comprises about 200 base pairs of the coding sequence of the GAPDH gene.

In some embodiments, the exogenous coding sequence of the GAPDH gene encodes a C-terminal fragment of a protein encoded by the GAPDH gene. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence of the GAPDH gene is less than 100% identical to the corresponding endogenous coding sequence of the GAPDH gene of the iPSC. In some embodiments, the exogenous coding sequence of the GAPDH gene has been codon optimized relative to the corresponding endogenous coding sequence of the GAPDH gene of the iPSC to remove a target site of a nuclease, e.g., a Cas. In some embodiments, the nuclease is a Cas (e.g., Cas9 or Cas12a), the exogenous coding sequence of the GAPDH gene includes at least one PAM site for the Cas, and the at least one PAM site (or all PAM sites) has been codon optimized or saturated with silent and/or missense mutations.

In some embodiments, the iPSC's genome comprises a regulatory element that enables expression of the gene product encoded by the GAPDH gene and the gene product of interest as separate gene products, optionally, wherein at least one of the gene products is a protein and the regulatory element enables expression of that protein separate from the other gene product. In some embodiments, the iPSC's genome comprises an IRES or 2A element located between the coding sequence of the GAPDH gene and the exogenous coding sequence for the gene product of interest.

In some embodiments, the iPSC's genome comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the iPSC's genome does not comprise a reporter gene, e.g., a fluorescent reporter gene or an antibiotic resistance gene.

In another aspect, the disclosure features an engineered iPSC comprising a genomic modification, wherein the genomic modification comprises an insertion of an exogenous knock-in cassette within an endogenous coding sequence of a GAPDH gene in the iPSC's genome, wherein the knock-in cassette comprises an exogenous coding sequence for a gene product of interest in frame with and downstream (3′) of an exogenous coding sequence or partial coding sequence encoding GAPDH, or a functional variant thereof, and wherein the iPSC expresses the gene product of interest and GAPDH, or a functional variant thereof, optionally wherein the gene product of interest and GAPDH are expressed from the endogenous GAPDH promoter.

In some embodiments, the exogenous coding sequence or partial coding sequence encoding GAPDH comprises about 2000, 1500, 1000, 750, 500, 400, 300, 200, 100, or 50 base pairs of the coding sequence of the GAPDH gene. In some embodiments, the exogenous coding sequence or partial coding sequence encoding GAPDH comprises about 200 base pairs of the coding sequence of the GAPDH gene.

In some embodiments, the exogenous coding sequence or partial coding sequence encoding GAPDH encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence encoding GAPDH is less than 100% identical to the corresponding endogenous coding sequence of the GAPDH gene of the iPSC. In some embodiments, the exogenous coding sequence or partial coding sequence encoding GAPDH has been codon optimized relative to the corresponding endogenous coding sequence of the GAPDH gene of the iPSC to remove a target site of a nuclease, e.g., a Cas. In some embodiments, the nuclease is a Cas (e.g., Cas9 or Cas12a), the exogenous coding sequence or partial coding sequence encoding GAPDH includes at least one PAM site for the Cas, and the at least one PAM site (or all PAM sites) has been codon optimized or saturated with silent and/or missense mutations.

In some embodiments, the iPSC's genome comprises a regulatory element that enables expression of the gene product encoded by the GAPDH gene and the gene product of interest as separate gene products, optionally, wherein at least one of the gene products is a protein and the regulatory element enables expression of that protein separate from the other gene product. In some embodiments, the iPSC's genome comprises an IRES or 2A element located between the coding sequence of the GAPDH gene and the exogenous coding sequence for the gene product of interest.

In some embodiments, the iPSC's genome comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the iPSC's genome does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the engineered iPSC comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the engineered iPSC comprises the first knock-in cassette at a first allele of the GAPDH gene and the second knock-in cassette at the second allele of the GAPDH gene. In some embodiments, the engineered iPSC expresses (a) the first and second gene products of interest, and (b) GAPDH, or a functional variant thereof.

In another aspect, the disclosure features an immune cell (e.g., an iNK cell or T cell) differentiated from an iPSC described herein.

In another aspect, the disclosure features any of the iPSCs (or iNK or T cell differentiated from an iPSC) described herein for use as a medicament and/or for use in the treatment of a disease, disorder or condition, e.g., a disease, disorder or condition described herein, e.g., a cancer, e.g., a cancer described herein.

In another aspect, the disclosure features an iPSC, or a population of iPSCs, produced by any of the methods described herein, or progeny thereof.

In another aspect, the disclosure features a system for editing the genome of an iPSC (or an iPSC in a population of iPSCs), the system comprising the iPSC (or the population of iPSC), a nuclease that causes a break within an endogenous coding sequence of a GAPDH gene of the iPSC, and a donor template that comprises a knock-in cassette comprising an exogenous coding sequence for a gene product of interest in frame with and downstream (3′) of an exogenous coding sequence or partial coding sequence of the GAPDH gene.

In some embodiments, after contacting the population of iPSCs with the nuclease and the donor template, at least about 60%, at least about 65%, at least about 70%, at least about 75%, at least about 80%, at least about 85%, at least about 90%, at least about 95%, or more, of the viable iPSCs of the population of iPSCs are genome-edited iPSCs, and/or about 40% or less, about 35% or less, about 30% or less, about 25% or less, about 20% or less, about 15% or less, about 10% or less, or about 5% or less, of the population of iPSCs lacking an integrated knock-in cassette are viable iPSCs. In some embodiments, after contacting the population of iPSCs with the nuclease and the donor template, at least about 80% of the viable iPSCs of the population of iPSCs are genome-edited iPSCs, and about 20% or less of the population of iPSCs lacking an integrated knock-in cassette are viable iPSCs. In some embodiments, after contacting the population of iPSCs with the nuclease and the donor template, at least about 60% of the viable iPSCs of the population of iPSCs are genome-edited iPSCs, and about 40% or less of the population of iPSCs lacking an integrated knock-in cassette are viable iPSCs. In some embodiments, after contacting the population of iPSCs with the nuclease and the donor template, at least about 90% of the viable iPSCs of the population of iPSCs are genome-edited iPSCs, and about 10% or less of the population of iPSCs lacking an integrated knock-in cassette are viable iPSCs. In some embodiments, after contacting the population of iPSCs with the nuclease and the donor template, at least about 95% of the viable iPSCs of the population of iPSCs are genome-edited iPSCs, and about 5% or less of the population of iPSCs lacking an integrated knock-in cassette are viable iPSCs.

In some embodiments, after contacting the iPSC or population of iPSCs with the nuclease and the donor template, if the knock-in cassette is not integrated into the genome of the iPSC by homology-directed repair (HDR) in the correct position or orientation, the iPSC no longer expresses GAPDH or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, after contacting the population of iPSCs with the nuclease and the donor template, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the system comprises a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, after contacting the population of iPSCs with the nuclease and the donor templates, the genome-edited iPSC comprises the first knock-in cassette at a first allele of the GAPDH gene and the second knock-in cassette at the second allele of the GAPDH gene. In some embodiments, the genome-edited iPSC expresses (a) the first and second gene products of interest, and (b) GAPDH, or a functional variant thereof.

In another aspect, the disclosure features a donor template comprising a knock-in cassette with an exogenous coding sequence for a gene product of interest in frame with and downstream (3′) of an exogenous coding sequence or partial coding sequence of a GAPDH gene.

In some embodiments, the donor template is for use in editing the genome of an iPSC by homology-directed repair (HDR).

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of a target site in the genome of the iPSC. In some embodiments, the donor template comprises a 3′ homology arm comprising a sequence homologous to a sequence located 3′ of a target site in the genome of the iPSC. In some embodiments, the donor template comprises a 5′ homology arm comprising a sequence homologous to a sequence located 5′ of a target site in the genome of the iPSC, and the donor template comprises a 3′ homology arm comprising a sequence homologous to a sequence located 3′ of a target site in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 10 amino acids in length. In some embodiments, the C-terminal fragment includes an amino acid sequence that is encoded by a region of the endogenous coding sequence of the GAPDH gene.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In another aspect, the disclosure features a method of producing a population of modified iPSCs, the method comprising contacting iPSCs with: (i) a nuclease that causes a break within an endogenous coding sequence of a GAPDH gene in a plurality of the iPSCs, and (ii) a donor template that comprises a knock-in cassette comprising an exogenous coding sequence for a gene product of interest in frame with and downstream (3′) of an exogenous coding sequence or partial coding sequence of the GAPDH gene, wherein the knock-in cassette is integrated into the genome of a plurality of the iPSCs by homology-directed repair (HDR) of the break, resulting in genome-edited iPSCs that expresses: (a) the gene product of interest, and (b) GAPDH, or a functional variant thereof, and wherein following the contacting step, at least about 60%, at least about 65%, at least about 70%, at least about 75%, at least about 80%, at least about 85%, at least about 90%, at least about 95%, or more, of the viable iPSCs are genome-edited iPSCs, and/or about 40% or less, about 35% or less, about 30% or less, about 25% or less, about 20% or less, about 15% or less, about 10% or less, or about 5% or less, of the iPSCs lacking an integrated knock-in cassette are viable iPSCs, thereby producing a population of modified iPSCs. In some embodiments, following the contacting step, at least about 80% of the viable iPSCs are genome-edited iPSCs, and about 20% or less of the iPSCs lacking an integrated knock-in cassette are viable iPSCs. In some embodiments, following the contacting step, at least about 60% of the viable iPSCs are genome-edited iPSCs, and about 40% or less of the iPSCs lacking an integrated knock-in cassette are viable iPSCs. In some embodiments, following the contacting step, at least about 90% of the viable iPSCs are genome-edited iPSCs, and about 10% or less of the iPSCs lacking an integrated knock-in cassette are viable iPSCs. In some embodiments, following the contacting step, at least about 95% of the viable iPSCs are genome-edited iPSCs, and about 5% or less of iPSCs lacking an integrated knock-in cassette are viable iPSCs.

In some embodiments, if the knock-in cassette is not integrated into the genome of the iPSC by homology-directed repair (HDR) in the correct position or orientation, the iPSC no longer expresses GAPDH, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSCs comprise knock-in cassettes at one or both alleles of the GAPDH gene. In some embodiments, the genome-edited iPSCs express (a) the first and second gene products of interest, and (b) GAPDH, or a functional variant thereof.

In some embodiments, the method comprises contacting iPSCs (or the population of iPSCs) with a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the genome-edited iPSCs comprise the first knock-in cassette at a first allele of the GAPDH gene and the second knock-in cassette at the second allele of the GAPDH gene. In some embodiments, the genome-edited iPSCs express (a) the first and second gene products of interest, and (b) GAPDH, or a functional variant thereof.

In another aspect, the disclosure features a method of selecting and/or identifying an iPSC comprising a knock-in of a gene product of interest within an endogenous coding sequence of a GAPDH gene in the iPSC, the method comprising contacting a population of iPSCs with: (i) a nuclease that causes a break within an endogenous coding sequence of a GAPDH gene in a plurality of the iPSCs, and (ii) a donor template that comprises a knock-in cassette comprising an exogenous coding sequence for a gene product of interest in frame with and downstream (3′) of an exogenous coding sequence or partial coding sequence of the GAPDH gene, wherein the knock-in cassette is integrated into the genome of a plurality of the iPSCs by homology-directed repair (HDR) of the break, and identifying a genome-edited iPSC within the population of iPSCs that expresses: (a) the gene product of interest, and (b) GAPDH, or a functional variant thereof.

In some embodiments, following the contacting step, at least about 60%, at least about 65%, at least about 70%, at least about 75%, at least about 80%, at least about 85%, at least about 90%, at least about 95%, or more, of the viable iPSCs of the population of iPSCs are genome-edited iPSCs, and/or about 40% or less, about 35% or less, about 30% or less, about 25% or less, about 20% or less, about 15% or less, about 10% or less, or about 5% or less, of the population of iPSCs lacking an integrated knock-in cassette are iPSCs. In some embodiments, following the contacting step, at least about 80% of the viable iPSCs of the population of iPSCs are genome-edited iPSCs, and about 20% or less of the population of iPSCs lacking an integrated knock-in cassette are viable iPSCs. In some embodiments, following the contacting step, at least about 60% of the viable iPSCs of the population of iPSCs are genome-edited iPSCs, and about 40% or less of the population of iPSCs lacking an integrated knock-in cassette are viable iPSCs. In some embodiments, following the contacting step, at least about 90% of the viable iPSCs of the population of iPSCs are genome-edited iPSCs, and about 10% or less of the population of iPSCs lacking an integrated knock-in cassette are viable iPSCs. In some embodiments, following the contacting step, at least about 95% of the viable iPSCs of the population of iPSCs are genome-edited iPSCs, and about 5% or less of the population of iPSCs lacking an integrated knock-in cassette are viable iPSCs.

In some embodiments, if the knock-in cassette is not integrated into the genome of the iPSC by homology-directed repair (HDR) in the correct position or orientation, the iPSC no longer expresses GAPDH, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the method comprises contacting the population of iPSCs with a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the genome-edited iPSCs comprise the first knock-in cassette at a first allele of the GAPDH gene and the second knock-in cassette at the second allele of the GAPDH gene. In some embodiments, the genome-edited iPSCs express (a) the first and second gene products of interest, and (b) GAPDH, or a functional variant thereof.

In another aspect, the disclosure features a method of editing the genome of an induced pluripotent stem cell (iPSC) (e.g., an iPSC in a population of iPSCs), the method comprising contacting the iPSC (or the population of iPSCs) with: (i) a nuclease that causes a break within an endogenous coding sequence of a glyceraldehyde 3-phosphate dehydrogenase (GAPDH) gene in the iPSC, and (ii) a donor template that comprises a knock-in cassette comprising an exogenous coding sequence for a gene product of interest in frame with and downstream (3′) of an exogenous coding sequence or partial coding sequence of the GAPDH gene, wherein the knock-in cassette is integrated into the genome of the iPSC by homology-directed repair (HDR) of the break, resulting in a genome-edited iPSC that expresses: (a) the gene product of interest, and (b) GAPDH, or a functional variant thereof, wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.

In some embodiments, following the contacting step, at least about 60%, at least about 65%, at least about 70%, at least about 75%, at least about 80%, at least about 85%, at least about 90%, at least about 95%, or more, of the viable iPSCs of the population of iPSCs are genome-edited iPSCs, and/or about 40% or less, about 35% or less, about 30% or less, about 25% or less, about 20% or less, about 15% or less, about 10% or less, or about 5% or less, of the population of iPSCs lacking an integrated knock-in cassette are viable iPSCs. In some embodiments, following the contacting step, at least about 80% of the viable iPSCs of the population of iPSCs are genome-edited iPSCs, and about 20% or less of the population of iPSCs lacking an integrated knock-in cassette are viable iPSCs. In some embodiments, following the contacting step, at least about 60% of the viable iPSCs of the population of iPSCs are genome-edited iPSCs, and about 40% or less of the population of iPSCs lacking an integrated knock-in cassette are viable iPSCs. In some embodiments, following the contacting step, at least about 90% of the viable iPSCs of the population of iPSCs are genome-edited iPSCs, and about 10% or less of the population of iPSCs lacking an integrated knock-in cassette are viable iPSCs. In some embodiments, following the contacting step, at least about 95% of the viable iPSCs of the population of iPSCs are genome-edited iPSCs, and about 5% or less of the population of iPSCs lacking an integrated knock-in cassette are viable iPSCs.

In some embodiments, if the knock-in cassette is not integrated into the genome of the iPSCs by homology-directed repair (HDR) in the correct position or orientation, the iPSCs no longer expresses GAPDH, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of a protein encoded by the GAPDH gene. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the method comprises contacting the iPSC (or the population of iPSCs) with a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the genome-edited iPSC comprises the first knock-in cassette at a first allele of the GAPDH gene and the second knock-in cassette at the second allele of the GAPDH gene. In some embodiments, the genome-edited iPSC expresses (a) the first and second gene products of interest, and (b) GAPDH, or a functional variant thereof.

In some embodiments, the genome-edited iPSC comprises multi-cistronic knock-ins (e.g., at one or both alleles of GAPDH gene) of two or more gene products of interest, e.g., one or more of the following gene products of interest, in order: CD16+IL15; IL15+CD16; CD16+CAR; CAR+CD16; IL15+CAR; CAR+IL15; CD16+(HLA-E or HLA-G or CD47); (HLA-E or HLA-G or CD47)+CD16; IL15+(HLA-E or HLA-G or CD47); (HLA-E or HLA-G or CD47)+IL15; CAR+(HLA-E or HLA-G or CD47); (HLA-E or HLA-G or CD47)+CAR. In some embodiments, the genome-edited iPSC comprises bi-allelic knock-ins (e.g., a first gene product of interest at a first allele of GAPDH gene, and a second gene product of interest at a second allele of GAPDH gene) of the following pairs of gene products of interest: CD16+IL15; IL15+CD16; CD16+CAR; CAR+CD16; IL15+CAR; CAR+IL15; CD16+(HLA-E or HLA-G or CD47); (HLA-E or HLA-G or CD47)+CD16; IL15+(HLA-E or HLA-G or CD47); (HLA-E or HLA-G or CD47)+IL15; CAR+(HLA-E or HLA-G or CD47); (HLA-E or HLA-G or CD47)+CAR.

In some embodiments, the method comprises contacting the iPSC (or the population of iPSCs) with a first a donor template that comprises a first knock-in cassette comprising a first exogenous coding sequence for a first gene product of interest in frame with and downstream (3′) of an exogenous coding sequence or partial coding sequence of a GAPDH gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, the genome-edited iPSC comprises the first knock-in cassette at one or both alleles of the GAPDH gene and the second knock-in cassette at one or both alleles of the second essential gene. In some embodiments, the genome-edited iPSC expresses (a) the first and second gene products of interest, (b) GAPDH, and (c) the gene product encoded by the second essential gene required for survival and/or proliferation of the iPSC, or a functional variant thereof. In some embodiments, the second essential gene is a gene listed in Table 3 or 4. In some embodiments, the second essential gene is TBP.

In another aspect, the disclosure features a genetically modified iPSC comprising a genome with an exogenous coding sequence for a gene product of interest in frame with and downstream (3′) of a coding sequence of a GAPDH gene, wherein at least part of the coding sequence of the GAPDH gene comprises an exogenous coding sequence, and wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.

In some embodiments, the exogenous coding sequence of the GAPDH gene comprises about 2000, 1500, 1000, 750, 500, 400, 300, 200, 100, or 50 base pairs of the coding sequence of the GAPDH gene. In some embodiments, the exogenous coding sequence of the GAPDH gene comprises about 200 base pairs of the coding sequence of the GAPDH gene.

In some embodiments, the exogenous coding sequence of the GAPDH gene encodes a C-terminal fragment of a protein encoded by the GAPDH gene. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence of the GAPDH gene is less than 100% identical to the corresponding endogenous coding sequence of the GAPDH gene of the iPSC. In some embodiments, the exogenous coding sequence of the GAPDH gene has been codon optimized relative to the corresponding endogenous coding sequence of the GAPDH gene of the iPSC to remove a target site of a nuclease, e.g., a Cas. In some embodiments, the nuclease is a Cas (e.g., Cas9 or Cas12a), the exogenous coding sequence of the GAPDH gene includes at least one PAM site for the Cas, and the at least one PAM site (or all PAM sites) has been codon optimized or saturated with silent and/or missense mutations.

In some embodiments, the iPSC's genome comprises a regulatory element that enables expression of the gene product encoded by the GAPDH gene and the gene product of interest as separate gene products, optionally, wherein at least one of the gene products is a protein and the regulatory element enables expression of that protein separate from the other gene product. In some embodiments, the iPSC's genome comprises an IRES or 2A element located between the coding sequence of the GAPDH gene and the exogenous coding sequence for the gene product of interest.

In some embodiments, the iPSC's genome comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the iPSC's genome does not comprise a reporter gene, e.g., a fluorescent reporter gene or an antibiotic resistance gene.

In another aspect, the disclosure features an engineered iPSC comprising a genomic modification, wherein the genomic modification comprises an insertion of an exogenous knock-in cassette within an endogenous coding sequence of a GAPDH gene in the iPSC's genome, wherein the knock-in cassette comprises an exogenous coding sequence for a gene product of interest in frame with and downstream (3′) of an exogenous coding sequence or partial coding sequence encoding GAPDH, or a functional variant thereof, wherein the iPSC expresses the gene product of interest and GAPDH, or a functional variant thereof, optionally wherein the gene product of interest and GAPDH are expressed from the endogenous GAPDH promoter, and wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.

In some embodiments, the exogenous coding sequence or partial coding sequence encoding GAPDH comprises about 2000, 1500, 1000, 750, 500, 400, 300, 200, 100, or 50 base pairs of the coding sequence of the GAPDH gene. In some embodiments, the exogenous coding sequence or partial coding sequence encoding GAPDH comprises about 200 base pairs of the coding sequence of the GAPDH gene.

In some embodiments, the exogenous coding sequence or partial coding sequence encoding GAPDH encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence encoding GAPDH is less than 100% identical to the corresponding endogenous coding sequence of the GAPDH gene of the iPSC. In some embodiments, the exogenous coding sequence or partial coding sequence encoding GAPDH has been codon optimized relative to the corresponding endogenous coding sequence of the GAPDH gene of the iPSC to remove a target site of a nuclease, e.g., a Cas. In some embodiments, the nuclease is a Cas (e.g., Cas9 or Cas12a), the exogenous coding sequence or partial coding sequence encoding GAPDH includes at least one PAM site for the Cas, and the at least one PAM site (or all PAM sites) has been codon optimized or saturated with silent and/or missense mutations.

In some embodiments, the iPSC's genome comprises a regulatory element that enables expression of the gene product encoded by the GAPDH gene and the gene product of interest as separate gene products, optionally, wherein at least one of the gene products is a protein and the regulatory element enables expression of that protein separate from the other gene product. In some embodiments, the iPSC's genome comprises an IRES or 2A element located between the coding sequence of the GAPDH gene and the exogenous coding sequence for the gene product of interest.

In some embodiments, the iPSC's genome comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the iPSC's genome does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the engineered iPSC comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the engineered iPSC comprises the first knock-in cassette at a first allele of the GAPDH gene and the second knock-in cassette at the second allele of the GAPDH gene. In some embodiments, the engineered iPSC expresses (a) the first and second gene products of interest, and (b) GAPDH, or a functional variant thereof.

In some embodiments, the engineered iPSC comprises multi-cistronic knock-ins (e.g., at one or both alleles of GAPDH gene) of two or more gene products of interest, e.g., one or more of the following gene products of interest, in order: CD16+IL15; IL15+CD16; CD16+CAR; CAR+CD16; IL15+CAR; CAR+IL15; CD16+(HLA-E or HLA-G or CD47); (HLA-E or HLA-G or CD47)+CD16; IL15+(HLA-E or HLA-G or CD47); (HLA-E or HLA-G or CD47)+IL15; CAR+(HLA-E or HLA-G or CD47); (HLA-E or HLA-G or CD47)+CAR. In some embodiments, the engineered iPSC comprises bi-allelic knock-ins (e.g., a first gene product of interest at a first allele of GAPDH gene, and a second gene product of interest at a second allele of GAPDH gene) of the following pairs of gene products of interest: CD16+IL15; IL15+CD16; CD16+CAR; CAR+CD16; IL15+CAR; CAR+IL15; CD16+(HLA-E or HLA-G or CD47); (HLA-E or HLA-G or CD47)+CD16; IL15+(HLA-E or HLA-G or CD47); (HLA-E or HLA-G or CD47)+IL15; CAR+(HLA-E or HLA-G or CD47); (HLA-E or HLA-G or CD47)+CAR.

In some embodiments, engineered iPSC comprises the first knock-in cassette at one or both alleles of the GAPDH gene and the second knock-in cassette at one or both alleles of a second essential gene. In some embodiments, the genome-edited iPSC expresses (a) the first and second gene products of interest, (b) GAPDH, and (c) the gene product encoded by the second essential gene required for survival and/or proliferation of the iPSC, or a functional variant thereof. In some embodiments, the second essential gene is a gene listed in Table 3 or 4. In some embodiments, the second essential gene is TBP.

In another aspect, the disclosure features an immune cell (e.g., an iNK cell or T cell) differentiated from an iPSC described herein.

In another aspect, the disclosure features any of the iPSCs (or iNK or T cell differentiated from an iPSC) described herein for use as a medicament and/or for use in the treatment of a disease, disorder or condition, e.g., a disease, disorder or condition described herein, e.g., a cancer, e.g., a cancer described herein.

In another aspect, the disclosure features an iPSC, or a population of iPSCs, produced by any of the methods described herein, or progeny thereof.

In another aspect, the disclosure features a system for editing the genome of an iPSC (or an iPSC in a population of iPSCs), the system comprising the iPSC (or the population of iPSC), a nuclease that causes a break within an endogenous coding sequence of a GAPDH gene of the iPSC, and a donor template that comprises a knock-in cassette comprising an exogenous coding sequence for a gene product of interest in frame with and downstream (3′) of an exogenous coding sequence or partial coding sequence of the GAPDH gene, and wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.

In some embodiments, after contacting the population of iPSCs with the nuclease and the donor template, at least about 60%, at least about 65%, at least about 70%, at least about 75%, at least about 80%, at least about 85%, at least about 90%, at least about 95%, or more, of the viable iPSCs of the population of iPSCs are genome-edited iPSCs, and/or about 40% or less, about 35% or less, about 30% or less, about 25% or less, about 20% or less, about 15% or less, about 10% or less, or about 5% or less, of the population of iPSCs lacking an integrated knock-in cassette are viable iPSCs. In some embodiments, after contacting the population of iPSCs with the nuclease and the donor template, at least about 80% of the viable iPSCs of the population of iPSCs are genome-edited iPSCs, and about 20% or less of the population of iPSCs lacking an integrated knock-in cassette are viable iPSCs. In some embodiments, after contacting the population of iPSCs with the nuclease and the donor template, at least about 60% of the viable iPSCs of the population of iPSCs are genome-edited iPSCs, and about 40% or less of the population of iPSCs lacking an integrated knock-in cassette are viable iPSCs. In some embodiments, after contacting the population of iPSCs with the nuclease and the donor template, at least about 90% of the viable iPSCs of the population of iPSCs are genome-edited iPSCs, and about 10% or less of the population of iPSCs lacking an integrated knock-in cassette are viable iPSCs. In some embodiments, after contacting the population of iPSCs with the nuclease and the donor template, at least about 95% of the viable iPSCs of the population of iPSCs are genome-edited iPSCs, and about 5% or less of the population of iPSCs lacking an integrated knock-in cassette are viable iPSCs.

In some embodiments, after contacting the iPSC or population of iPSCs with the nuclease and the donor template, if the knock-in cassette is not integrated into the genome of the iPSC by homology-directed repair (HDR) in the correct position or orientation, the iPSC no longer expresses GAPDH or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, after contacting the population of iPSCs with the nuclease and the donor template, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the system comprises a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, after contacting the population of iPSCs with the nuclease and the donor templates, the genome-edited iPSC comprises the first knock-in cassette at a first allele of the GAPDH gene and the second knock-in cassette at the second allele of the GAPDH gene. In some embodiments, the genome-edited iPSC expresses (a) the first and second gene products of interest, and (b) GAPDH, or a functional variant thereof.

In some embodiments, after contacting the population of iPSCs with the nuclease and the donor template or templates, the iPSCs comprise multi-cistronic knock-ins (e.g., at one or both alleles of GAPDH gene) of two or more gene products of interest, e.g., one or more of the following gene products of interest, in order: CD16+IL15; IL15+CD16; CD16+CAR; CAR+CD16; IL15+CAR; CAR+IL15; CD16+(HLA-E or HLA-G or CD47); (HLA-E or HLA-G or CD47)+CD16; IL15+(HLA-E or HLA-G or CD47); (HLA-E or HLA-G or CD47)+IL15; CAR+(HLA-E or HLA-G or CD47); (HLA-E or HLA-G or CD47)+CAR. In some embodiments, the iPSCs comprise bi-allelic knock-ins (e.g., a first gene product of interest at a first allele of GAPDH gene, and a second gene product of interest at a second allele of GAPDH gene) of the following pairs of gene products of interest: CD16+IL15; IL15+CD16; CD16+CAR; CAR+CD16; IL15+CAR; CAR+IL15; CD16+(HLA-E or HLA-G or CD47); (HLA-E or HLA-G or CD47)+CD16; IL15+(HLA-E or HLA-G or CD47); (HLA-E or HLA-G or CD47)+IL15; CAR+(HLA-E or HLA-G or CD47); (HLA-E or HLA-G or CD47)+CAR.

In some embodiments, the iPSCs comprise the first knock-in cassette at one or both alleles of the GAPDH gene and the second knock-in cassette at one or both alleles of a second essential gene. In some embodiments, the IPSCs express (a) the first and second gene products of interest, (b) GAPDH, and (c) the gene product encoded by the second essential gene required for survival and/or proliferation of the iPSC, or a functional variant thereof. In some embodiments, the second essential gene is a gene listed in Table 3 or 4. In some embodiments, the second essential gene is TBP.

In another aspect, the disclosure features a donor template comprising a knock-in cassette with an exogenous coding sequence for a gene product of interest in frame with and downstream (3′) of an exogenous coding sequence or partial coding sequence of a GAPDH gene, wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.

In some embodiments, the donor template is for use in editing the genome of an iPSC by homology-directed repair (HDR).

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of a target site in the genome of the iPSC. In some embodiments, the donor template comprises a 3′ homology arm comprising a sequence homologous to a sequence located 3′ of a target site in the genome of the iPSC. In some embodiments, the donor template comprises a 5′ homology arm comprising a sequence homologous to a sequence located 5′ of a target site in the genome of the iPSC, and the donor template comprises a 3′ homology arm comprising a sequence homologous to a sequence located 3′ of a target site in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 10 amino acids in length. In some embodiments, the C-terminal fragment includes an amino acid sequence that is encoded by a region of the endogenous coding sequence of the GAPDH gene.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In another aspect, the disclosure features a method of producing a population of modified iPSCs, the method comprising contacting iPSCs with: (i) a nuclease that causes a break within an endogenous coding sequence of a GAPDH gene in a plurality of the iPSCs, and (ii) a donor template that comprises a knock-in cassette comprising an exogenous coding sequence for a gene product of interest in frame with and downstream (3′) of an exogenous coding sequence or partial coding sequence of the GAPDH gene, wherein the knock-in cassette is integrated into the genome of a plurality of the iPSCs by homology-directed repair (HDR) of the break, resulting in genome-edited iPSCs that expresses: (a) the gene product of interest, and (b) GAPDH, or a functional variant thereof, wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof, and wherein following the contacting step, at least about 60%, at least about 65%, at least about 70%, at least about 75%, at least about 80%, at least about 85%, at least about 90%, at least about 95%, or more, of the viable iPSCs are genome-edited iPSCs, and/or about 40% or less, about 35% or less, about 30% or less, about 25% or less, about 20% or less, about 15% or less, about 10% or less, or about 5% or less, of the iPSCs lacking an integrated knock-in cassette are viable iPSCs, thereby producing a population of modified iPSCs. In some embodiments, following the contacting step, at least about 80% of the viable iPSCs are genome-edited iPSCs, and about 20% or less of the iPSCs lacking an integrated knock-in cassette are viable iPSCs. In some embodiments, following the contacting step, at least about 60% of the viable iPSCs are genome-edited iPSCs, and about 40% or less of the iPSCs lacking an integrated knock-in cassette are viable iPSCs. In some embodiments, following the contacting step, at least about 90% of the viable iPSCs are genome-edited iPSCs, and about 10% or less of the iPSCs lacking an integrated knock-in cassette are viable iPSCs. In some embodiments, following the contacting step, at least about 95% of the viable iPSCs are genome-edited iPSCs, and about 5% or less of iPSCs lacking an integrated knock-in cassette are viable iPSCs.

In some embodiments, if the knock-in cassette is not integrated into the genome of the iPSC by homology-directed repair (HDR) in the correct position or orientation, the iPSC no longer expresses GAPDH, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSCs comprise knock-in cassettes at one or both alleles of the GAPDH gene. In some embodiments, the genome-edited iPSCs express (a) the first and second gene products of interest, and (b) GAPDH, or a functional variant thereof.

In some embodiments, the method comprises contacting iPSCs (or the population of iPSCs) with a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the genome-edited iPSCs comprise the first knock-in cassette at a first allele of the GAPDH gene and the second knock-in cassette at the second allele of the GAPDH gene. In some embodiments, the genome-edited iPSCs express (a) the first and second gene products of interest, and (b) GAPDH, or a functional variant thereof.

In some embodiments, the genome-edited iPSCs comprise multi-cistronic knock-ins (e.g., at one or both alleles of GAPDH gene) of two or more gene products of interest, e.g., one or more of the following gene products of interest, in order: CD16+IL15; IL15+CD16; CD16+CAR; CAR+CD16; IL15+CAR; CAR+IL15; CD16+(HLA-E or HLA-G or CD47); (HLA-E or HLA-G or CD47)+CD16; IL15+(HLA-E or HLA-G or CD47); (HLA-E or HLA-G or CD47)+IL15; CAR+(HLA-E or HLA-G or CD47); (HLA-E or HLA-G or CD47)+CAR. In some embodiments, the genome-edited iPSCs comprise bi-allelic knock-ins (e.g., a first gene product of interest at a first allele of GAPDH gene, and a second gene product of interest at a second allele of GAPDH gene) of the following pairs of gene products of interest: CD16+IL15; IL15+CD16; CD16+CAR; CAR+CD16; IL15+CAR; CAR+IL15; CD16+(HLA-E or HLA-G or CD47); (HLA-E or HLA-G or CD47)+CD16; IL15+(HLA-E or HLA-G or CD47); (HLA-E or HLA-G or CD47)+IL15; CAR+(HLA-E or HLA-G or CD47); (HLA-E or HLA-G or CD47)+CAR.

In some embodiments, the method comprises contacting iPSCs (or the population of iPSCs) with a first a donor template that comprises a first knock-in cassette comprising a first exogenous coding sequence for a first gene product of interest in frame with and downstream (3′) of an exogenous coding sequence or partial coding sequence of a GAPDH gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, the genome-edited iPSC comprises the first knock-in cassette at one or both alleles of the GAPDH gene and the second knock-in cassette at one or both alleles of the second essential gene. In some embodiments, the genome-edited iPSC expresses (a) the first and second gene products of interest, (b) GAPDH, and (c) the gene product encoded by the second essential gene required for survival and/or proliferation of the iPSC, or a functional variant thereof. In some embodiments, the second essential gene is a gene listed in Table 3 or 4. In some embodiments, the second essential gene is TBP.

In another aspect, the disclosure features a method of selecting and/or identifying an iPSC comprising a knock-in of a gene product of interest within an endogenous coding sequence of a GAPDH gene in the iPSC, the method comprising contacting a population of iPSCs with: (i) a nuclease that causes a break within an endogenous coding sequence of a GAPDH gene in a plurality of the iPSCs, and (ii) a donor template that comprises a knock-in cassette comprising an exogenous coding sequence for a gene product of interest in frame with and downstream (3′) of an exogenous coding sequence or partial coding sequence of the GAPDH gene, wherein the knock-in cassette is integrated into the genome of a plurality of the iPSCs by homology-directed repair (HDR) of the break, and identifying a genome-edited iPSC within the population of iPSCs that expresses: (a) the gene product of interest, and (b) GAPDH, or a functional variant thereof, wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.

In some embodiments, following the contacting step, at least about 60%, at least about 65%, at least about 70%, at least about 75%, at least about 80%, at least about 85%, at least about 90%, at least about 95%, or more, of the viable iPSCs of the population of iPSCs are genome-edited iPSCs, and/or about 40% or less, about 35% or less, about 30% or less, about 25% or less, about 20% or less, about 15% or less, about 10% or less, or about 5% or less, of the population of iPSCs lacking an integrated knock-in cassette are iPSCs. In some embodiments, following the contacting step, at least about 80% of the viable iPSCs of the population of iPSCs are genome-edited iPSCs, and about 20% or less of the population of iPSCs lacking an integrated knock-in cassette are viable iPSCs. In some embodiments, following the contacting step, at least about 60% of the viable iPSCs of the population of iPSCs are genome-edited iPSCs, and about 40% or less of the population of iPSCs lacking an integrated knock-in cassette are viable iPSCs. In some embodiments, following the contacting step, at least about 90% of the viable iPSCs of the population of iPSCs are genome-edited iPSCs, and about 10% or less of the population of iPSCs lacking an integrated knock-in cassette are viable iPSCs. In some embodiments, following the contacting step, at least about 95% of the viable iPSCs of the population of iPSCs are genome-edited iPSCs, and about 5% or less of the population of iPSCs lacking an integrated knock-in cassette are viable iPSCs.

In some embodiments, if the knock-in cassette is not integrated into the genome of the iPSC by homology-directed repair (HDR) in the correct position or orientation, the iPSC no longer expresses GAPDH, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the method comprises contacting the population of iPSCs with a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the genome-edited iPSCs comprise the first knock-in cassette at a first allele of the GAPDH gene and the second knock-in cassette at the second allele of the GAPDH gene. In some embodiments, the genome-edited iPSCs express (a) the first and second gene products of interest, and (b) GAPDH, or a functional variant thereof.

In some embodiments, the genome-edited iPSCs comprise multi-cistronic knock-ins (e.g., at one or both alleles of GAPDH gene) of two or more gene products of interest, e.g., one or more of the following gene products of interest, in order: CD16+IL15; IL15+CD16; CD16+CAR; CAR+CD16; IL15+CAR; CAR+IL15; CD16+(HLA-E or HLA-G or CD47); (HLA-E or HLA-G or CD47)+CD16; IL15+(HLA-E or HLA-G or CD47); (HLA-E or HLA-G or CD47)+IL15; CAR+(HLA-E or HLA-G or CD47); (HLA-E or HLA-G or CD47)+CAR. In some embodiments, the genome-edited iPSCs comprise bi-allelic knock-ins (e.g., a first gene product of interest at a first allele of GAPDH gene, and a second gene product of interest at a second allele of GAPDH gene) of the following pairs of gene products of interest: CD16+IL15; IL15+CD16; CD16+CAR; CAR+CD16; IL15+CAR; CAR+IL15; CD16+(HLA-E or HLA-G or CD47); (HLA-E or HLA-G or CD47)+CD16; IL15+(HLA-E or HLA-G or CD47); (HLA-E or HLA-G or CD47)+IL15; CAR+(HLA-E or HLA-G or CD47); (HLA-E or HLA-G or CD47)+CAR.

In some embodiments, the method comprises contacting iPSCs (or population of iPSCs) with a first a donor template that comprises a first knock-in cassette comprising a first exogenous coding sequence for a first gene product of interest in frame with and downstream (3′) of an exogenous coding sequence or partial coding sequence of a GAPDH gene, and with a second donor template that comprises a second knock-in cassette comprising a second exogenous coding sequence for a second gene product of interest in frame with and downstream (3′) of an exogenous coding sequence or partial coding sequence of a second essential gene. In some embodiments, the genome-edited iPSCs comprise the first knock-in cassette at one or both alleles of the GAPDH gene and the second knock-in cassette at one or both alleles of the second essential gene. In some embodiments, the genome-edited iPSCs express (a) the first and second gene products of interest, (b) GAPDH, and (c) the gene product encoded by the second essential gene required for survival and/or proliferation of the iPSCs, or a functional variant thereof. In some embodiments, the second essential gene is a gene listed in Table 3 or 4. In some embodiments, the second essential gene is TBP.

In another aspect, the disclosure features a method of editing the genome of an induced pluripotent stem cell (iPSC) (e.g., an iPSC in a population of iPSCs), the method comprising contacting the iPSC (or the population of iPSCs) with: (i) a nuclease that causes a break within an endogenous coding sequence of a glyceraldehyde 3-phosphate dehydrogenase (GAPDH) gene in the iPSC, and (ii) a donor template that comprises a knock-in cassette comprising an exogenous coding sequence for a gene product of interest in frame with and downstream (3′) of an exogenous coding sequence or partial coding sequence of the GAPDH gene, wherein the knock-in cassette is integrated into the genome of the iPSC by homology-directed repair (HDR) of the break, resulting in a genome-edited iPSC that expresses: (a) the gene product of interest, and (b) GAPDH, or a functional variant thereof, wherein the gene product of interest is PD-L1 or leukocyte surface antigen cluster of differentiation CD47 (CD47).

In some embodiments, following the contacting step, at least about 60%, at least about 65%, at least about 70%, at least about 75%, at least about 80%, at least about 85%, at least about 90%, at least about 95%, or more, of the viable iPSCs of the population of iPSCs are genome-edited iPSCs, and/or about 40% or less, about 35% or less, about 30% or less, about 25% or less, about 20% or less, about 15% or less, about 10% or less, or about 5% or less, of the population of iPSCs lacking an integrated knock-in cassette are viable iPSCs. In some embodiments, following the contacting step, at least about 80% of the viable iPSCs of the population of iPSCs are genome-edited iPSCs, and about 20% or less of the population of iPSCs lacking an integrated knock-in cassette are viable iPSCs. In some embodiments, following the contacting step, at least about 60% of the viable iPSCs of the population of iPSCs are genome-edited iPSCs, and about 40% or less of the population of iPSCs lacking an integrated knock-in cassette are viable iPSCs. In some embodiments, following the contacting step, at least about 90% of the viable iPSCs of the population of iPSCs are genome-edited iPSCs, and about 10% or less of the population of iPSCs lacking an integrated knock-in cassette are viable iPSCs. In some embodiments, following the contacting step, at least about 95% of the viable iPSCs of the population of iPSCs are genome-edited iPSCs, and about 5% or less of the population of iPSCs lacking an integrated knock-in cassette are viable iPSCs.

In some embodiments, if the knock-in cassette is not integrated into the genome of the iPSCs by homology-directed repair (HDR) in the correct position or orientation, the iPSCs no longer expresses GAPDH, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of a protein encoded by the GAPDH gene. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the method comprises contacting the iPSC (or the population of iPSCs) with a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the genome-edited iPSC comprises the first knock-in cassette at a first allele of the GAPDH gene and the second knock-in cassette at the second allele of the GAPDH gene. In some embodiments, the genome-edited iPSC expresses (a) the first and second gene products of interest, and (b) GAPDH, or a functional variant thereof.

In some embodiments, the genome-edited iPSC comprises multi-cistronic knock-ins (e.g., at one or both alleles of GAPDH gene) of two or more gene products of interest, e.g., one or more of the following gene products of interest, in order: PD-L1+CD47; or CD47+PD-L1. In some embodiments, the genome-edited iPSC comprises bi-allelic knock-ins (e.g., a first gene product of interest at a first allele of GAPDH gene, and a second gene product of interest at a second allele of GAPDH gene) of the following pairs of gene products of interest: PD-L1+CD47.

In another aspect, the disclosure features a genetically modified iPSC comprising a genome with an exogenous coding sequence for a gene product of interest in frame with and downstream (3′) of a coding sequence of a GAPDH gene, wherein at least part of the coding sequence of the GAPDH gene comprises an exogenous coding sequence, and wherein the gene product of interest is PD-L1 or leukocyte surface antigen cluster of differentiation CD47 (CD47).

In some embodiments, the exogenous coding sequence of the GAPDH gene comprises about 2000, 1500, 1000, 750, 500, 400, 300, 200, 100, or 50 base pairs of the coding sequence of the GAPDH gene. In some embodiments, the exogenous coding sequence of the GAPDH gene comprises about 200 base pairs of the coding sequence of the GAPDH gene.

In some embodiments, the exogenous coding sequence of the GAPDH gene encodes a C-terminal fragment of a protein encoded by the GAPDH gene. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence of the GAPDH gene is less than 100% identical to the corresponding endogenous coding sequence of the GAPDH gene of the iPSC. In some embodiments, the exogenous coding sequence of the GAPDH gene has been codon optimized relative to the corresponding endogenous coding sequence of the GAPDH gene of the iPSC to remove a target site of a nuclease, e.g., a Cas. In some embodiments, the nuclease is a Cas (e.g., Cas9 or Cas12a), the exogenous coding sequence of the GAPDH gene includes at least one PAM site for the Cas, and the at least one PAM site (or all PAM sites) has been codon optimized or saturated with silent and/or missense mutations.

In some embodiments, the iPSC's genome comprises a regulatory element that enables expression of the gene product encoded by the GAPDH gene and the gene product of interest as separate gene products, optionally, wherein at least one of the gene products is a protein and the regulatory element enables expression of that protein separate from the other gene product. In some embodiments, the iPSC's genome comprises an IRES or 2A element located between the coding sequence of the GAPDH gene and the exogenous coding sequence for the gene product of interest.

In some embodiments, the iPSC's genome comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the iPSC's genome does not comprise a reporter gene, e.g., a fluorescent reporter gene or an antibiotic resistance gene.

In another aspect, the disclosure features an engineered iPSC comprising a genomic modification, wherein the genomic modification comprises an insertion of an exogenous knock-in cassette within an endogenous coding sequence of a GAPDH gene in the iPSC's genome, wherein the knock-in cassette comprises an exogenous coding sequence for a gene product of interest in frame with and downstream (3′) of an exogenous coding sequence or partial coding sequence encoding GAPDH, or a functional variant thereof, wherein the iPSC expresses the gene product of interest and GAPDH, or a functional variant thereof, optionally wherein the gene product of interest and GAPDH are expressed from the endogenous GAPDH promoter, and wherein the gene product of interest is PD-L1 or leukocyte surface antigen cluster of differentiation CD47 (CD47).

In some embodiments, the exogenous coding sequence or partial coding sequence encoding GAPDH comprises about 2000, 1500, 1000, 750, 500, 400, 300, 200, 100, or 50 base pairs of the coding sequence of the GAPDH gene. In some embodiments, the exogenous coding sequence or partial coding sequence encoding GAPDH comprises about 200 base pairs of the coding sequence of the GAPDH gene.

In some embodiments, the exogenous coding sequence or partial coding sequence encoding GAPDH encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence encoding GAPDH is less than 100% identical to the corresponding endogenous coding sequence of the GAPDH gene of the iPSC. In some embodiments, the exogenous coding sequence or partial coding sequence encoding GAPDH has been codon optimized relative to the corresponding endogenous coding sequence of the GAPDH gene of the iPSC to remove a target site of a nuclease, e.g., a Cas. In some embodiments, the nuclease is a Cas (e.g., Cas9 or Cas12a), the exogenous coding sequence or partial coding sequence encoding GAPDH includes at least one PAM site for the Cas, and the at least one PAM site (or all PAM sites) has been codon optimized or saturated with silent and/or missense mutations.

In some embodiments, the iPSC's genome comprises a regulatory element that enables expression of the gene product encoded by the GAPDH gene and the gene product of interest as separate gene products, optionally, wherein at least one of the gene products is a protein and the regulatory element enables expression of that protein separate from the other gene product. In some embodiments, the iPSC's genome comprises an IRES or 2A element located between the coding sequence of the GAPDH gene and the exogenous coding sequence for the gene product of interest.

In some embodiments, the iPSC's genome comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the iPSC's genome does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the engineered iPSC comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the engineered iPSC comprises the first knock-in cassette at a first allele of the GAPDH gene and the second knock-in cassette at the second allele of the GAPDH gene. In some embodiments, the engineered iPSC expresses (a) the first and second gene products of interest, and (b) GAPDH, or a functional variant thereof.

In some embodiments, the engineered iPSC comprises multi-cistronic knock-ins (e.g., at one or both alleles of GAPDH gene) of two or more gene products of interest, e.g., one or more of the following gene products of interest, in order: PD-L1+CD47; CD47+PD-L1. In some embodiments, the engineered iPSC comprises bi-allelic knock-ins (e.g., a first gene product of interest at a first allele of GAPDH gene, and a second gene product of interest at a second allele of GAPDH gene) of PD-L1+CD47.

In another aspect, the disclosure features an immune cell (e.g., an iNK cell or T cell) differentiated from an iPSC described herein.

In another aspect, the disclosure features any of the iPSCs (or iNK or T cell differentiated from an iPSC) described herein for use as a medicament and/or for use in the treatment of a disease, disorder or condition, e.g., a disease, disorder or condition described herein, e.g., a cancer, e.g., a cancer described herein.

In another aspect, the disclosure features an iPSC, or a population of iPSCs, produced by any of the methods described herein, or progeny thereof.

In another aspect, the disclosure features a system for editing the genome of an iPSC (or an iPSC in a population of iPSCs), the system comprising the iPSC (or the population of iPSC), a nuclease that causes a break within an endogenous coding sequence of a GAPDH gene of the iPSC, and a donor template that comprises a knock-in cassette comprising an exogenous coding sequence for a gene product of interest in frame with and downstream (3′) of an exogenous coding sequence or partial coding sequence of the GAPDH gene, and wherein the gene product of interest is PD-L1 or leukocyte surface antigen cluster of differentiation CD47 (CD47).

In some embodiments, after contacting the population of iPSCs with the nuclease and the donor template, at least about 60%, at least about 65%, at least about 70%, at least about 75%, at least about 80%, at least about 85%, at least about 90%, at least about 95%, or more, of the viable iPSCs of the population of iPSCs are genome-edited iPSCs, and/or about 40% or less, about 35% or less, about 30% or less, about 25% or less, about 20% or less, about 15% or less, about 10% or less, or about 5% or less, of the population of iPSCs lacking an integrated knock-in cassette are viable iPSCs. In some embodiments, after contacting the population of iPSCs with the nuclease and the donor template, at least about 80% of the viable iPSCs of the population of iPSCs are genome-edited iPSCs, and about 20% or less of the population of iPSCs lacking an integrated knock-in cassette are viable iPSCs. In some embodiments, after contacting the population of iPSCs with the nuclease and the donor template, at least about 60% of the viable iPSCs of the population of iPSCs are genome-edited iPSCs, and about 40% or less of the population of iPSCs lacking an integrated knock-in cassette are viable iPSCs. In some embodiments, after contacting the population of iPSCs with the nuclease and the donor template, at least about 90% of the viable iPSCs of the population of iPSCs are genome-edited iPSCs, and about 10% or less of the population of iPSCs lacking an integrated knock-in cassette are viable iPSCs. In some embodiments, after contacting the population of iPSCs with the nuclease and the donor template, at least about 95% of the viable iPSCs of the population of iPSCs are genome-edited iPSCs, and about 5% or less of the population of iPSCs lacking an integrated knock-in cassette are viable iPSCs.

In some embodiments, after contacting the iPSC or population of iPSCs with the nuclease and the donor template, if the knock-in cassette is not integrated into the genome of the iPSC by homology-directed repair (HDR) in the correct position or orientation, the iPSC no longer expresses GAPDH or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, after contacting the population of iPSCs with the nuclease and the donor template, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the system comprises a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, after contacting the population of iPSCs with the nuclease and the donor templates, the genome-edited iPSC comprises the first knock-in cassette at a first allele of the GAPDH gene and the second knock-in cassette at the second allele of the GAPDH gene. In some embodiments, the genome-edited iPSC expresses (a) the first and second gene products of interest, and (b) GAPDH, or a functional variant thereof.

In some embodiments, after contacting the population of iPSCs with the nuclease and the donor template or templates, the iPSCs comprise multi-cistronic knock-ins (e.g., at one or both alleles of GAPDH gene) of two or more gene products of interest, e.g., one or more of the following gene products of interest, in order: PD-L1+CD47; CD47+PD-L1.

In another aspect, the disclosure features a donor template comprising a knock-in cassette with an exogenous coding sequence for a gene product of interest in frame with and downstream (3′) of an exogenous coding sequence or partial coding sequence of a GAPDH gene, wherein the gene product of interest is PD-L1 or leukocyte surface antigen cluster of differentiation CD47 (CD47).

In some embodiments, the donor template is for use in editing the genome of an iPSC by homology-directed repair (HDR).

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of a target site in the genome of the iPSC. In some embodiments, the donor template comprises a 3′ homology arm comprising a sequence homologous to a sequence located 3′ of a target site in the genome of the iPSC. In some embodiments, the donor template comprises a 5′ homology arm comprising a sequence homologous to a sequence located 5′ of a target site in the genome of the iPSC, and the donor template comprises a 3′ homology arm comprising a sequence homologous to a sequence located 3′ of a target site in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 10 amino acids in length. In some embodiments, the C-terminal fragment includes an amino acid sequence that is encoded by a region of the endogenous coding sequence of the GAPDH gene.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In another aspect, the disclosure features a method of producing a population of modified iPSCs, the method comprising contacting iPSCs with: (i) a nuclease that causes a break within an endogenous coding sequence of a GAPDH gene in a plurality of the iPSCs, and (ii) a donor template that comprises a knock-in cassette comprising an exogenous coding sequence for a gene product of interest in frame with and downstream (3′) of an exogenous coding sequence or partial coding sequence of the GAPDH gene, wherein the knock-in cassette is integrated into the genome of a plurality of the iPSCs by homology-directed repair (HDR) of the break, resulting in genome-edited iPSCs that expresses: (a) the gene product of interest, and (b) GAPDH, or a functional variant thereof, wherein the gene product of interest is PD-L1 or leukocyte surface antigen cluster of differentiation CD47 (CD47), and wherein following the contacting step, at least about 60%, at least about 65%, at least about 70%, at least about 75%, at least about 80%, at least about 85%, at least about 90%, at least about 95%, or more, of the viable iPSCs are genome-edited iPSCs, and/or about 40% or less, about 35% or less, about 30% or less, about 25% or less, about 20% or less, about 15% or less, about 10% or less, or about 5% or less, of the iPSCs lacking an integrated knock-in cassette are viable iPSCs, thereby producing a population of modified iPSCs. In some embodiments, following the contacting step, at least about 80% of the viable iPSCs are genome-edited iPSCs, and about 20% or less of the iPSCs lacking an integrated knock-in cassette are viable iPSCs. In some embodiments, following the contacting step, at least about 60% of the viable iPSCs are genome-edited iPSCs, and about 40% or less of the iPSCs lacking an integrated knock-in cassette are viable iPSCs. In some embodiments, following the contacting step, at least about 90% of the viable iPSCs are genome-edited iPSCs, and about 10% or less of the iPSCs lacking an integrated knock-in cassette are viable iPSCs. In some embodiments, following the contacting step, at least about 95% of the viable iPSCs are genome-edited iPSCs, and about 5% or less of iPSCs lacking an integrated knock-in cassette are viable iPSCs.

In some embodiments, if the knock-in cassette is not integrated into the genome of the iPSC by homology-directed repair (HDR) in the correct position or orientation, the iPSC no longer expresses GAPDH, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSCs comprise knock-in cassettes at one or both alleles of the GAPDH gene. In some embodiments, the genome-edited iPSCs express (a) the first and second gene products of interest, and (b) GAPDH, or a functional variant thereof.

In some embodiments, the method comprises contacting iPSCs (or the population of iPSCs) with a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the genome-edited iPSCs comprise the first knock-in cassette at a first allele of the GAPDH gene and the second knock-in cassette at the second allele of the GAPDH gene. In some embodiments, the genome-edited iPSCs express (a) the first and second gene products of interest, and (b) GAPDH, or a functional variant thereof.

In some embodiments, the genome-edited iPSCs comprise multi-cistronic knock-ins (e.g., at one or both alleles of GAPDH gene) of two or more gene products of interest, e.g., one or more of the following gene products of interest, in order: PD-L1+CD47; CD47+PD-L1.

In another aspect, the disclosure features a method of selecting and/or identifying an iPSC comprising a knock-in of a gene product of interest within an endogenous coding sequence of a GAPDH gene in the iPSC, the method comprising contacting a population of iPSCs with: (i) a nuclease that causes a break within an endogenous coding sequence of a GAPDH gene in a plurality of the iPSCs, and (ii) a donor template that comprises a knock-in cassette comprising an exogenous coding sequence for a gene product of interest in frame with and downstream (3′) of an exogenous coding sequence or partial coding sequence of the GAPDH gene, wherein the knock-in cassette is integrated into the genome of a plurality of the iPSCs by homology-directed repair (HDR) of the break, and identifying a genome-edited iPSC within the population of iPSCs that expresses: (a) the gene product of interest, and (b) GAPDH, or a functional variant thereof, wherein the gene product of interest is PD-L1 or leukocyte surface antigen cluster of differentiation CD47 (CD47).

In some embodiments, following the contacting step, at least about 60%, at least about 65%, at least about 70%, at least about 75%, at least about 80%, at least about 85%, at least about 90%, at least about 95%, or more, of the viable iPSCs of the population of iPSCs are genome-edited iPSCs, and/or about 40% or less, about 35% or less, about 30% or less, about 25% or less, about 20% or less, about 15% or less, about 10% or less, or about 5% or less, of the population of iPSCs lacking an integrated knock-in cassette are iPSCs. In some embodiments, following the contacting step, at least about 80% of the viable iPSCs of the population of iPSCs are genome-edited iPSCs, and about 20% or less of the population of iPSCs lacking an integrated knock-in cassette are viable iPSCs. In some embodiments, following the contacting step, at least about 60% of the viable iPSCs of the population of iPSCs are genome-edited iPSCs, and about 40% or less of the population of iPSCs lacking an integrated knock-in cassette are viable iPSCs. In some embodiments, following the contacting step, at least about 90% of the viable iPSCs of the population of iPSCs are genome-edited iPSCs, and about 10% or less of the population of iPSCs lacking an integrated knock-in cassette are viable iPSCs. In some embodiments, following the contacting step, at least about 95% of the viable iPSCs of the population of iPSCs are genome-edited iPSCs, and about 5% or less of the population of iPSCs lacking an integrated knock-in cassette are viable iPSCs.

In some embodiments, if the knock-in cassette is not integrated into the genome of the iPSC by homology-directed repair (HDR) in the correct position or orientation, the iPSC no longer expresses GAPDH, or a functional variant thereof.

In some embodiments, the break is a double-strand break.

In some embodiments, the break is located within the last 2000, 1500, 1000, 750, 500, 400, 300, 200, 100, or 50 base pairs of the endogenous coding sequence of the GAPDH gene. In some embodiments, the break is located within the last 200 base pairs of the endogenous coding sequence of the GAPDH gene. In some embodiments, the break is located within the last exon of the GAPDH gene.

In some embodiments, the nuclease is highly efficient, e.g., capable of editing at least about 60%, at least about 65%, at least about 75%, at least about 80%, at least about 85%, at least about 90%, at least about 95%, or more, of iPSCs contacted with the nuclease. In some embodiments, the nuclease is a zinc finger nuclease (ZFN), a transcription activator-like effector nuclease (TALEN) or a meganuclease. In some embodiments, the nuclease is a CRISPR/Cas nuclease and the method further comprises contacting the iPSC (or the population of iPSCs) with a guide molecule for the CRISPR/Cas nuclease. In some embodiments, the nuclease is a Cas9 or a Cas12a nuclease, or a variant thereof (e.g., a nuclease comprising the amino acid sequence of any one of SEQ ID NOs: 58-66). In some embodiments, the guide molecule comprises a targeting domain sequence that is complementary to a portion of the endogenous coding sequence of the GAPDH gene. In some embodiments, the guide molecule comprises a targeting domain sequence that differs by no more than 3 nucleotides from a sequence that is complementary to a portion of the endogenous coding sequence of the GAPDH gene. In some embodiments, the guide molecule specifically binds to the portion of the endogenous coding sequence of the GAPDH gene. In some embodiments, the guide molecule does not bind to an endogenous coding sequence of another gene, e.g., a different essential gene. In some embodiments, the guide comprises a nucleotide sequence of any one of SEQ ID NOs: 94-157 and 225-1885.

In some embodiments, the donor template is a donor DNA template, optionally wherein the donor DNA template is double-stranded. In some embodiments, the donor DNA template is a plasmid, optionally wherein the plasmid has not been linearized.

In some embodiments, the donor template comprises homology arms on either side of the knock-in cassette. In some embodiments, the donor template comprises a 5′ homology arm comprising a sequence homologous to a sequence located 5′ of the break in the genome of the iPSC. In some embodiments, the donor template comprises a 3′ homology arm comprising a sequence homologous to a sequence located 3′ of the break in the genome of the iPSC. In some embodiments, the donor template comprises a 5′ homology arm comprising a sequence homologous to a sequence located 5′ of the break in the genome of the iPSC, and the donor template comprises a 3′ homology arm comprising a sequence homologous to a sequence located 3′ of the break in the genome of the iPSC.

In some embodiments, the knock-in cassette comprises a regulatory element that enables expression of GAPDH and the gene product of interest as separate gene products, optionally, wherein at least one of the gene products is a protein and the regulatory element enables expression of that protein separate from the other gene product. In some embodiments, the knock-in cassette comprises an IRES or 2A element located between the exogenous coding sequence or partial coding sequence of the GAPDH gene and the exogenous coding sequence for the gene product of interest. In some embodiments, the 2A element is a T2A element (e.g., EGRGSLLTCGDVEENPGP), a P2A element (e.g., ATNFSLLKQAGDVEENPGP), a E2A element (e.g., QCTNYALLKLAGDVESNPGP), or an F2A element (e.g., VKQTLNFDLLKLAGDVESNPGP). In some embodiments, the knock-in cassette further comprises a sequence encoding a linker peptide upstream of the 2A element. In some embodiments, the linker peptide comprises the amino acid sequence GSG.

In some embodiments, the knock-in cassette comprises a polyadenylation sequence, and optionally a 3′ UTR sequence, downstream of the exogenous coding sequence for the gene product of interest, and, if a 3′UTR sequence is present, the 3′UTR sequence is positioned 3′ of the exogenous coding sequence and 5′ of the polyadenylation sequence.

In some embodiments, the exogenous partial coding sequence of the GAPDH gene in the knock-in cassette encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence of the GAPDH gene in the knock-in cassette is less than 100% identical to the corresponding endogenous coding sequence of the GAPDH gene of the iPSC. In some embodiments, the exogenous coding sequence or partial coding sequence of the GAPDH gene in the knock-in cassette has been codon optimized relative to the corresponding endogenous coding sequence of the GAPDH gene of the iPSC to remove a target site of the nuclease, to reduce the likelihood of homologous recombination after integration of the knock-in cassette into the genome of the iPSC, or to increase expression of GAPDH and/or the gene product of interest after integration of the knock-in cassette into the genome of the iPSC.

In some embodiments, the nuclease is a Cas (e.g., Cas9 or Cas12a), the exogenous coding sequence or partial coding sequence of the GAPDH gene in the knock-in cassette includes at least one PAM site for the Cas, and the at least one PAM site (or all PAM sites) has been codon optimized or saturated with silent and/or missense mutations.

In some embodiments, the donor template does not comprise a reporter gene, e.g., a fluorescent reporter gene or an antibiotic resistance gene.

In some embodiments, the knock-in cassette is a multi-cistronic (e.g., bi-cistronic) knock-in cassette comprising exogenous coding sequences for two or more gene products of interest. In some embodiments, the knock-in cassette comprises a first exogenous coding sequence for a first gene product of interest, a linker (e.g., T2A, P2A, and/or IRES), and a second exogenous coding sequence for a second gene product of interest. In some embodiments, the genome-edited iPSC comprises knock-in cassettes at one or both alleles of the GAPDH gene. In some embodiments, the genome-edited iPSC expresses (a) the first and second gene products of interest, and (b) GAPDH, or a functional variant thereof.

In some embodiments, the method comprises contacting the population of iPSCs with a first a donor template that comprises a first knock-in cassette comprising a first exogenous coding sequence for a first gene product of interest in frame with and downstream (3′) of an exogenous coding sequence or partial coding sequence of the GAPDH gene, and with a second donor template that comprises a second knock-in cassette comprising a second exogenous coding sequence for a second gene product of interest in frame with and downstream (3′) of an exogenous coding sequence or partial coding sequence of the GAPDH gene. In some embodiments, the genome-edited iPSCs comprise the first knock-in cassette at a first allele of the GAPDH gene and the second knock-in cassette at the second allele of the GAPDH gene. In some embodiments, the genome-edited iPSCs express (a) the first and second gene products of interest, and (b) GAPDH, or a functional variant thereof.

In some embodiments, the genome-edited iPSCs comprise multi-cistronic knock-ins (e.g., at one or both alleles of GAPDH gene) of two or more gene products of interest, e.g., one or more of the following gene products of interest, in order: PD-L1+CD47; CD47+PD-L1.

In another aspect, the disclosure features a method of generating a genetically modified mammalian cell comprising a coding sequence for a gene product of interest at a pre-determined genomic position, comprising: providing at least one donor template comprising the coding sequence for a gene product of interest flanked by a first homologous arm and a second homology arm, wherein the first and second homology arms are essentially homologous to a first genomic region (GR) and a second GR, respectively, wherein the first GR and the second GR are adjacent to and flank a pre-determined genomic position in an exon of an essential gene in a mammalian cell, wherein the cell becomes inviable if the exon is disrupted; providing a gene editing system containing a nuclease that is targeted to the pre-determined genomic position; introducing the at least one donor template and the gene editing system into a population of mammalian cells; culturing the population of mammalian cells; and identifying a surviving cell that comprises the coding sequence for the gene product of interest, wherein the identified surviving cell is a genetically modified mammalian cell comprising the coding sequence for the gene product of interest at the pre-determined genomic position. In another aspect, the disclosure features a method of selecting a mammalian cell comprising a coding sequence for a gene product of interest that has integrated precisely at a pre-determined genomic position, comprising: providing at least one donor template comprising the coding sequence for the gene product of interest flanked by a first homology arm and a second homology arm, wherein the first and second homology arms are essentially homologous to a first genomic region (GR) and a second GR, respectively, wherein the first GR and the second GR are adjacent to and flank a pre-determined genomic position in an exon of an essential gene in a mammalian cell, wherein the cell becomes inviable if the exon is disrupted; providing a gene editing system containing a nuclease that is targeted to the pre-determined genomic position; introducing the donor template and the gene editing system into a population of mammalian cells; culturing the population of mammalian cells; and identifying a surviving cell that comprises the coding sequence for a gene product of interest, wherein the identified surviving cell comprises the coding sequence for a gene product of interest integrated precisely at the pre-determined genomic position.

In some embodiments, the exon is the last or penultimate exon of the essential gene if the essential gene has more than one exon. In some embodiments, the pre-determined genomic position in the exon of the essential gene is within about 200 bps upstream of a stop codon, or within about 200 bps downstream of a start codon, of the essential gene.

In some embodiments, the gene editing system is a meganuclease based system, a zinc finger nuclease (ZFN) based system, a transcription activator-like effector based nuclease (TALEN) system, a CRISPR based system, or a NgAgo based system.

In some embodiments, the gene editing system is a CRISPR based system comprising a nuclease, or an mRNA or DNA encoding a nuclease, and a guide RNA (gRNA) that targets the pre-determined genomic position, optionally wherein the gene editing system is a ribonucleoprotein (RNP) complex comprising the nuclease and the gRNA.

In some embodiments, the nuclease is Cas5, Cash, Cas7, Cas9 (optionally saCas9 or spCas9), Cas12a, or Csm1.

In some embodiments, the essential gene is selected from the gene loci listed in Table 3 or 4. In some embodiments, the essential gene is GAPDH, RPL13A, RPL7, or RPLP0 gene.

In some embodiments, the first homology arm and/or the second homology arm comprise a silent PAM blocking mutation or a codon modification that prevents cleavage of the donor template by the nuclease such that the essential gene locus, once modified, is not cleaved by the nuclease.

In some embodiments, the coding sequence for the gene product of interest is linked in frame to the essential gene sequence through a coding sequence for a self-cleaving peptide, or the coding sequence for the gene product of interest contains an internal ribosomal entry site (IRES) at the 5′ end.

In some embodiments, the gene product of interest is a therapeutic protein (optionally an antibody, an engineered antigen receptor, or an antigen-binding fragment thereof), an immunomodulatory protein, a reporter protein, or a safety switch signal.

In some embodiments, the method further comprises contacting the population of mammalian cells with an inhibitor of non-homologous end joining.

In some embodiments, the population of mammalian cells are human cells. In some embodiments, the populations of mammalian cells are pluripotent stem cells (PSCs). In some embodiments, the PSCs are embryonic stem cells or induced PSCs (iPSCs).

In some embodiments, the method comprises providing more than one donor template. In some embodiments, each donor template is targeted to the essential gene. In some embodiments, each donor template comprises a different genomic sequence. In some embodiments, each donor template comprises coding sequence for more than one gene product of interest.

In some embodiments, the genomic sequences from one donor template are incorporated into one allele of the essential gene and the genomic sequences from the other donor template are incorporated into the other allele of the essential gene. In some embodiments, each donor template comprises coding sequence for more than one gene product of interest.

In some embodiments, each donor template comprises at least one safety switch. In some embodiments, each donor template comprises at least one component of a safety switch. In some embodiments, the safety switch requires dimerization to function as a suicide switch.

In some embodiments, the method further comprising the additional steps of providing to the surviving cells, the gene editing system containing a nuclease that is targeted to the pre-determined genomic position; optionally reintroducing the at least one donor template, to obtain a second population of mammalian cells; culturing the second population of mammalian cells; and identifying a surviving cell from the second population of mammalian cells that comprises the coding sequences for gene products of interest from the donor templates; wherein the identified surviving cell from the second population of mammalian cells is a genetically modified mammalian cell comprising the coding sequences for gene products of interest from donor templates at the pre-determined genomic position.

In some embodiments, the percentage of surviving cells from the second culturing step comprising the coding sequences for gene products of interest is enriched at least four-fold from the surviving cells from the first culturing step comprising the coding sequences for gene products of interest. In some embodiments, the percentage of surviving cells from the second culturing step comprising the coding sequences for gene products of interest from the donor templates is at least 2%.

In some embodiments, the method further comprises separating a mammalian cell comprising the coding sequences for gene products of interest from the donor templates. In some embodiments, the method further comprises growing the mammalian cell comprising the coding sequences for gene products of interest from the donor templates into a plurality of cells comprising the coding sequences for gene products of interest from the donor templates.

In some embodiments, the population of mammalian cells are PSCs. In some embodiments, the PSCs are embryonic stem cells or iPSCs.

In another aspect, the disclosure features a genetically engineered cell obtainable by any of the methods described herein. In some embodiments, the genetically engineered cell is a PSC. In some embodiments, the genetically engineered cell is an iPSC.

In another aspect, the disclosure features a method of obtaining a differentiated cell, comprising culturing a genetically engineered iPSC obtainable by any of the methods described herein in a culture medium that allows differentiation of the iPSC into the differentiated cell, or a genetically modified differentiated cell obtained by such method. In some embodiments, the differentiated cell is an immune cell, optionally selected from a T cell, a T cell expressing a chimeric antigen receptor (CAR), a suppressive T cell, a myeloid cell, a dendritic cell, and an immunosuppressive macrophage; a cell in the nervous system, optionally selected from dopaminergic neuron, a microglial cell, an oligodendrocyte, an astrocyte, a cortical neuron, a spinal or oculomotor neuron, an enteric neuron, a Placode-derived cell, a Schwann cell, and a trigeminal or sensory neuron; a cell in the ocular system, optionally selected from a retinal pigment epithelial cell, a photoreceptor cone cell, a photoreceptor rod cell, a bipolar cell, and a ganglion cell; a cell in the cardiovascular system, optionally selected from a cardiomyocyte, an endothelial cell, and a nodal cell; or a cell in the metabolic system, optionally selected from a hepatocyte, a cholangiocyte, and a pancreatic beta cell. In some embodiments, the differentiated cell is a human cell.

In another aspect, the disclosure features a pharmaceutical composition comprising any of the cells described herein. In another aspect, the disclosure features a method of treating a human patient in need thereof, comprising introducing the pharmaceutical composition to the patient, wherein the pharmaceutical composition comprises differentiated human cells. In another aspect, the disclosure features the pharmaceutical composition for use in treating a human patient in need thereof, wherein the pharmaceutical composition comprises differentiated human cells. In another aspect, the disclosure features use of the pharmaceutical composition for the manufacture of a medicament in treating a human patient in need thereof, wherein the pharmaceutical composition comprises differentiated human cells. In some embodiments, the differentiated human cells are autologous or allogenic cells.

In another aspect, the disclosure features a system for editing the genome of a mammalian cell, the system comprising a population of mammalian cells, a nuclease that causes a break within an endogenous coding sequence of an essential gene of the mammalian cell, and a plurality of donor templates each comprising a knock-in cassette comprising an exogenous coding sequence for a gene product of interest in frame with and downstream (3′) of an exogenous coding sequence or partial coding sequence of the essential gene, and wherein after contacting the population of mammalian cells with the nuclease and the donor templates, and optionally contacting the population of mammalian cells with the nuclease and optionally the donor templates a second time, at least about 2% of the viable cells of the population of mammalian cells are genome-edited cells that expresses the gene products of interest from the plurality of donor templates. In some embodiments, the essential gene is GAPDH.

In some embodiments, the mammalian cell is a PSC. In some embodiments, the mammalian cell is an iPSC.

In some embodiments, the break is a double-strand break. In some embodiments, the break is located within the last 1000, 500, 400, 300, 200, 100 or 50 base pairs of the coding sequence of the GAPDH gene. In some embodiments, the break is located within the last exon of the GAPDH gene.

In some embodiments, the nuclease is a CRISPR/Cas nuclease and the system further comprises a guide molecule for the CRISPR/Cas nuclease. In some embodiments, the nuclease is a zinc finger nuclease (ZFN), a transcription activator-like effector nuclease (TALEN) or a meganuclease.

In some embodiments, the donor templates are donor DNA templates, optionally wherein the donor DNA templates are double-stranded. In some embodiments, the donor templates comprise homology arms on either side of the exogenous coding sequences. In some embodiments, the homology arms correspond to sequences located on either side of the break in the genome of the mammalian cell.

### BRIEF DESCRIPTION OF THE DRAWING

The teachings described herein will be more fully understood from the following description of various exemplary embodiments, when read together with the accompanying drawing. It should be understood that the drawing described below is for illustration purposes only and is not intended to limit the scope of the present teachings in any way.

FIG. 1 shows the locations on the GAPDH gene where exemplary AsCpf1 (AsCas12a) guide RNAs bind, and the results of screening the exemplary guide RNAs that target the GAPDH gene three days after transfection. Results are from gDNA from living cells.

FIG. 2 shows results of screening the exemplary AsCpf1 (AsCas12a) guide RNAs that target the GAPDH gene, three days after transfection. Results are from gDNA from living cells.

FIG. 3A shows an exemplary integration strategy that targets an essential gene according to certain embodiments of the present disclosure. In particular embodiments, introducing a double strand break using CRISPR gene editing (e.g., by Cas12a or Cas9) within a terminal exon (e.g., within about 500 bp upstream (5′) of the stop codon of the essential gene) and administering a donor plasmid with homology arms designed to mediate homology directed repair (HDR) at the cleavage site, results in a population of viable cells carrying a cargo of interest integrated at the essential gene locus. Those cells that were edited the CRISPR nuclease, but failed to undergo integration of the cargo at the essential gene locus, do not survive.

FIG. 3B shows an exemplary integration strategy that targets the GAPDH gene according to certain embodiments of the present disclosure. Although FIG. 3B shows a strategy wherein the GAPDH gene is modified in an induced pluripotent stem cell (iPSC), this strategy can be applied to a variety of cell types, including primary cells, stem cells, and cells differentiated from iPSCs.

FIG. 3C shows an exemplary integration strategy that targets the GAPDH gene according to certain embodiments of the present disclosure. The diagram shows that the only cells that should survive over time are those cells that underwent targeted integration of a cassette that restores the GAPDH locus and includes a cargo of interest, as well as unedited cells. The population of unedited cells following CRISPR editing should be small if the nuclease and guide RNA are highly effective at cleaving the essential gene target site and introduce indels that significantly reduce the function of the essential gene product.

FIG. 3D shows an exemplary integration strategy that targets an essential gene according to certain embodiments of the present disclosure. In particular embodiments, introducing a double strand break using CRISPR gene editing (e.g., by Cas12a or Cas9) to target a 5′ exon (e.g., within about 500 bp downstream (3′) of a start codon of the essential gene) and administering a donor plasmid with homology arms designed to mediate homology directed repair (HDR) at the cleavage site, results in a population of viable cells carrying a cargo of interest integrated at the essential gene locus. Those cells that were edited the CRISPR nuclease, but failed to undergo integration of the cargo at the essential gene locus, do not survive.

FIG. 4 shows editing efficiency at different concentrations (0.625 μM to 4 μM) of an exemplary AsCpf1 (AsCas12a) guide RNA that targets the GAPDH gene.

FIG. 5 shows the knock-in (KI) efficiency of a CD47 encoding “cargo” in the GAPDH gene 4 days post-electroporation when the dsDNA plasmid (“PLA”) was also present. Knock-in efficiency was measured with two different concentrations of the plasmid. Knock-in was measured using ddPCR targeting the 3′ positions of the knock-in “cargo”.

FIG. 6 shows the knock-in efficiency of a CD47 encoding “cargo” in the GAPDH gene 9 days post-electroporation when the dsDNA plasmid was also present. Knock-in was measured using ddPCR both targeting the 5′ and 3′ positions of the knock-in “cargo”, increasing the reliability of the result.

FIG. 7 maps AsCpf1 (AsCas12a) guide RNAs that target terminal exons of the RPLP0 gene.

FIG. 8 maps AsCpf1 (AsCas12a) guide RNAs that target terminal exons of the RPLP0 gene.

FIG. 9 maps AsCpf1 (AsCas12a) guide RNAs that target terminal exons of the RPL13A gene.

FIG. 10 maps AsCpf1 (AsCas12a) guide RNAs that target terminal exons of the RPL13A gene.

FIG. 11 maps AsCpf1 (AsCas12a) guide RNAs that target terminal exons of the RPL7 gene.

FIG. 12 maps AsCpf1 (AsCas12a) guide RNAs that target terminal exons of the RPL7 gene.

FIG. 13 shows the efficiency of integration of a knock-in cassette, comprising a GFP protein encoding “cargo” sequence, into the GAPDH locus of iPSCs, measured 7 days following transfection. (A) Depicts exemplary microscopy (brightfield and fluorescent) images, and (B) depicts exemplary flow cytometry data. Images and flow cytometry data depict insertion rates for cargo transfection alone (PLA1593 or PLA1651) compared to cargo and guide RNA transfections (RSQ22337+PLA1593 or RSQ24570+PLA1651), additionally, insertion rates with an exemplary exonic coding region targeting guide RNA with appropriate cargo (RSQ22337+PLA1593) are compared to insertion rates with an intronic targeting guide RNA with appropriate cargo (RSQ24570+PLA1651).

FIG. 14A depicts a schematic representation of a bicistronic knock-in cassette (e.g., comprising two cistrons separated by a linker) for insertion into the GAPDH locus, the leading GAPDH Exon 9 coding region and exogenous sequences encoding proteins of interest are separated by linker sequences, the second GAPDH allele can comprise a target knock-in cassette insertion, indels, or is wild type (WT).

FIG. 14B depicts a schematic representation of bi-allelic knock-in cassettes for insertion into the GAPDH locus. Exogenous “cargo” sequences encoding proteins of interest are located on different knock-in cassettes, for each construct, the leading GAPDH Exon 9 coding region is separated from an exogenous sequence encoding a protein of interest by a linker sequence.

FIG. 15A depicts a schematic representation of a bicistronic knock-in cassette for insertion into the GAPDH locus, with the leading GAPDH Exon 9 coding region and exogenous sequences encoding GFP and mCherry separated by linker sequences P2A, T2A, and/or IRES.

FIG. 15B is a panel of exemplary microscopic images (brightfield and fluorescent) of iPSCs nine days following nucleofection of RNPs comprising RSQ22337 (SEQ ID NO: 95) targeting GAPDH and Cas12a (SEQ ID NO: 62) and a bicistronic knock-in cassette comprising “cargo” sequence encoding GFP and mCherry molecules inserted at the GAPDH locus. iPSCs comprising exemplary “cargo” molecules PLA1582 (comprising donor template SEQ ID NO: 41) with linkers P2A and T2A, PLA1583 (comprising donor template SEQ ID NO: 42) with linkers T2A and P2A, and PLA1584 (comprising donor template SEQ ID NO: 43) with linkers T2A and IRES are shown. Results show that at least two different cargos can be inserted in a bicistronic manner and expression is detectable irrespective of linker type used. All images were taken at 2×100 μm on a Keyence Microscope.

FIG. 15C depicts expression quantification (Y axis) of exemplary “cargo” molecules GFP and mCherry from various bicistronic molecules comprising the described linker pairs (X axis). mCherry as a sole “cargo” protein was utilized as a relative control.

FIG. 16A depicts exemplary flow cytometry data for bi-allelic GFP and mCherry knock-in at the GAPDH gene.

FIG. 16B depicts fluorescence imaging of cell populations prior to flow cytometry analysis following bi-allelic GFP and mCherry knock-in at the GAPDH gene.

FIG. 16C are histograms depicting exemplary flow cytometry analysis data for bi-allelic GFP and mCherry knock-in at the GAPDH gene. Cells were nucleofected with 0.5 μM RNPs comprising Cas12a (SEQ ID NO: 62) and RSQ22337 (SEQ ID NO: 95), and 2.5 μg (5 trials) or 5 μg (1 trial) GFP and mCherry donor templates.

FIG. 17A depicts exemplary flow cytometry data for GFP expression in iPSCs seven days after being transfected with a gRNA and an appropriate donor template comprising a knock-in cassette with a “cargo” sequence encoding GFP that was recombined into various loci.

FIG. 17B depicts the percentage of cells having editing events as measured by Inference of CRISPR Edits (ICE) assays 48 hours after being transfected with the noted gRNA.

FIG. 17C depicts relative integrated “cargo” (GFP) expression intensity as determined by flow cytometry conducted with an FITC channel to filter GFP signal for iPSCs transfected with the noted exemplary gRNA and knock-in cassette combinations.

FIG. 18 depicts exemplary flow cytometry data highlighting the efficiency of integration of a donor template comprising a knock-in cassette comprising a GFP protein encoding “cargo” sequence, into the TBP locus of iPSCs.

FIG. 19 is exemplary ddPCR results describing knock-in cassette integration ratios in GAPDH or TBP alleles in an iPSC population.

FIG. 20 is a histogram representation of exemplary flow cytometry data for AAV6 mediated knock-in of GFP into T cells using RNPs comprising RSQ22337 targeting GAPDH and Cas12a (SEQ ID NO: 62) at various concentrations of RNP and various AAV6 multiplicity of infection (MOI) rates (vg/cell) measured seven days after electroporation and transduction. The Y axis represents percentage cell population expressing GFP, while the X axis depicts AAV6 MOI.

FIG. 21 is a histogram representation of exemplary flow cytometry data depicting cell viability following AAV6 mediated knock-in of GFP at the GAPDH gene in differentiated cells. Depicted is T cell viability four days after AAV6 mediated transduction of a GFP cargo and electroporated with 1 μM RNPs comprising RSQ22337 and Cas12a (SEQ ID NO: 62); the Y axis notes cell viability as a function of total cell population, while the X axis lists various MOIs used to transduce the cells.

FIG. 22A depicts exemplary flow cytometry charts for a population of T cells transduced by AAV6 comprising a knock-in GFP cargo targeting GAPDH at 5E4 MOI and transformed with 4 μM RNP comprising Cas12a (SEQ NO: 62) and RSQ22337.

FIG. 22B depicts exemplary control experiment flow cytometry charts for T cells that were not transduced by AAV6, but solely transformed with 4 μM RNP comprising Cas12a (SEQ NO: 62) and RSQ22337.

FIG. 23 are histograms depicting exemplary flow cytometry data for AAV6 mediated knock-in of GFP into T cells at either the GAPDH locus using RNPs comprising RSQ22337 and Cas12a (SEQ ID NO: 62), or at the TRAC locus. Integration constructs each comprised homology arms approximately 500 bp in length, and T cells were transduced with the same concentration of RNP and AAV MOI. The mean and standard deviation of three independent biological replicates is shown, significant differences in targeted integration were observed (p=0.0022 using unpaired t-test).

FIG. 24A is a histogram depicting the knock-in efficiency of CD16 encoding “cargo” integrated at the GAPDH gene of iPSCs. Targeting integration (TI) was measured at day 0 and day 19 of bulk edited cell populations using ddPCR targeting the 5′ (5′ assay) and 3′ (3′ assay) positions of the knock-in cargo.

FIG. 24B is a histogram depicting the genotypes of iPSC clones with CD16 encoding “cargo” integrated at the GAPDH gene, measured using ddPCR targeting the 5′ (5′ CDN probe) and 3′ (3′ PolyA probe) positions of the knock-in cargo. Shown are results for four exemplary cell lines, two lines were classified as homozygous knock-in with targeted integration (TI) rates of 88.5% (clone 1) and 90.5% (clone 2) respectively, and two lines were classified as heterozygous knock-in with TI rates of 45.6% (clone 1) and 46.5% (clone 2) respectively.

FIG. 25A depicts exemplary flow cytometry data from day 32 of homozygous clone 1 CD16 knock-in iPSCs differentiation into iNKs. The data highlights the efficiency of integration and high expression (e.g., approximately 98%) of a knock-in cassette comprising a CD16 protein encoding “cargo” sequence, into the GAPDH gene of iPSCs. In addition, the data shows knock-in of a “cargo” at the GADPH gene does not inhibit the differentiation process, as represented by high CD56+CD45+ population proportions.

FIG. 25B depicts exemplary flow cytometry data from day 32 of homozygous clone 2 CD16 knock-in iPSCs differentiation into iNKs. The data highlights the efficiency of integration and expression of a knock-in cassette comprising a CD16 protein encoding “cargo” sequence, into the GAPDH gene of iPSCs.

FIG. 25C depicts exemplary flow cytometry data from day 32 of heterozygous clone 1 CD16 knock-in iPSCs differentiation into iNKs. The data highlights the efficiency of integration and high expression (e.g., approximately 97.8%) of a knock-in cassette comprising a CD16 protein encoding “cargo” sequence, into the GAPDH gene of iPSCs.

FIG. 25D depicts exemplary flow cytometry data from day 32 of heterozygous clone 2 CD16 knock-in iPSCs differentiation into iNKs. The data highlights the efficiency of integration and expression of a knock-in cassette comprising a CD16 protein encoding “cargo” sequence, into the GAPDH gene of iPSCs.

FIG. 26 is a schematic representation of an exemplary solid tumor cell killing assay, depicting the use of knock-in iPSCs differentiated into iNK cells to kill 3D spheroids created from a cancer cell line (e.g., SK-OV-3 ovarian cancer cells). Antibodies and/or cytokines may optionally be added during the 3D spheroid killing stage.

FIG. 27A shows the results of a solid tumor killing assay as described in FIG. 26. Homozygous clones comprising CD16 knock-in at the GAPDH gene were differentiated into iNK cells and functioned to reduce tumor cell spheroid size, particularly following the addition of an antibody, e.g., 10 μg/mL trastuzumab; addition of an antibody promotes antibody dependent cellular cytotoxicity (ADCC) and tumor cell killing by iNKs. Control “WT PCS” cells were bulk unedited parental clones that were electroporated without RNPs or plasmids, and at the same stage of iNK cell differentiation as test cells. The Y axis depicts normalized total integrated red object intensity, a proxy for tumor cell abundance, while the X axis depicts the Effector to Target cell (E:T) ratio.

FIG. 27B shows the results of a solid tumor killing assay as described in FIG. 26. Heterozygous clones comprising CD16 knock-in at the GAPDH gene were differentiated into iNK cells and functioned to reduce tumor cell spheroid size, particularly following the addition of an antibody, e.g., 10 μg/mL trastuzumab; addition of an antibody promotes ADCC and tumor cell killing by iNKs. Control “WT PCS” cells were bulk unedited parental clones that were electroporated without RNPs or plasmids, and at the same stage of iNK cell differentiation as test cells. The Y axis depicts normalized total integrated red object intensity, a proxy for tumor cell abundance, while the X axis depicts the E:T ratio.

FIG. 28 shows the results of an in-vitro serial killing assay, where homozygous or heterozygous clones comprising CD16 knock-in at the GAPDH gene were differentiated into iNK cells and were serially challenged with hematological cancer cells (e.g., Raji cells), with or without the addition of antibody 0.1 μg/mL rituximab. The X axis represents time (0-598 hr.) with an additional tumor cell bolus (5,000 cells) being added approximately every 48 hours, the Y axis represents killing efficacy as measured by normalized total red object area (e.g., presence of tumor cells). Star (*) denotes onset of addition of 0.1 μg/mL rituximab in previously rituximab absent trials. The data shows that edited iNK cells (CD16 knock-in at GAPDH gene; clones “Homo_C1”, “Homo_C2”, “Het_C1”, and “Het_C2”) continue to kill hematological cancer cells while unedited (“PCS”) or control edited iNKs (“GFP Bulk”) derived from parental iPSCs lose this function at equivalent time points.

FIG. 29 depicts a correlation (R² of 0.768) between CD16 expression and reduction in tumor spheroid size at an Effector to Target (E:T) ratio of 3.16:1. Shown are differentiated iNK cells derived from either iPSC bulk edited cells or iPSC individual clones with CD16 knock-in at the GAPDH gene. The Y axis represents normalized tumor cell killing values, while the X axis represents the percentage of a cell population expressing CD16.

FIG. 30A is a histogram depicting exemplary ddPCR data measured at day 9 post nucleofection of two different iPSC lines with plasmids and 2 μM RNPs comprising RSQ22337 targeting the GAPDH gene and Cas12a (SEQ ID NO: 62), for knock-in of CD16 cargo, a CAR cargo, or a biallelic GFP/mCherry cargo into the GAPDH gene.

FIG. 30B depicts exemplary flow cytometry data from iPSC lines edited with plasmids and 2 μM RNPs comprising RSQ22337 targeting the GAPDH gene and Cas12a (SEQ ID NO: 62), for knock-in of CXCR2 cargo into the GAPDH gene (GAPDH::CXCR2) or control iPSCs transformed with RNP only (Wild-type). CXCR2 expression is noted on the X axis, edited cells expressing CXCR2 was 29.2% of the bulk edited cell population, while surface expression of CXCR2 was 8.53% of the bulk edited cell populations.

FIG. 31 is a histogram depicting the knock-in efficiency of a series of knock-in cassette cargo sequences such as CD16-P2A-CAR, CD16-IRES-CAR, CAR-P2A-CD16, CAR-IRES-CD16, and mbIL-15 into the GAPDH gene using RNPs comprising RSQ22337 targeting the GAPDH gene and Cas12a (SEQ ID NO: 62), measured on day 0 post-electroporation measured using ddPCR targeting the 5′ (5′ CDN probe) and 3′ (3′ PolyA probe) positions of the knock-in “cargo”.

FIG. 32 diagrammatically depicts a membrane-bound IL15.IL15Rα (mbIL-15) construct that can be utilized as a knock-in cargo sequence as described herein.

FIG. 33 is a histogram depicting the TI of mbIL-15 into the GAPDH gene over time when measured as a percentage of a bulk edited population. Shown are TI rates from iPSCs that that are on day 28 of the differentiation to iNK cell process.

FIG. 34A depicts exemplary flow cytometry data from bulk edited mbIL-15 GAPDH gene knock-in iPSC populations at day 39 of differentiation into iNKs.

FIG. 34B depicts exemplary flow cytometry data from bulk edited mbIL-15 GAPDH gene knock-in iPSC populations at day 39 of differentiation into iNKs.

FIG. 34C shows surface expression phenotypes (measured as a percentage of the population) of bulk edited mbIL-15 GAPDH gene knock-in iPSC populations being differentiated into iNK cells as compared to parental clone cells also being differentiated into iNK cells (“WT”) at day 32, day 39, day 42, and day 49 of iPSC differentiation.

FIG. 35 shows the results from two in-vitro tumor cell killing assays. Two biological replicates of bulk edited iPSC populations (S1 and S2) comprising mbIL-15 knock-in at the GAPDH gene were differentiated into iNK cells (day 56 of differentiation for S2, and day 63 of differentiation for 51) and functioned to reduce hematological cancer cells (e.g., Raji cells) fluorescence signal when compared to WT parental cells also differentiated into iNK cells, measured in the absence or presence of 10 μg/mL rituximab, E:T ratios of 1 (A) or 2.5 (B); (experiments performed in duplicate, R1 and R2).

FIG. 36 shows the results of a solid tumor killing assay as described in FIG. 26. Two biological replicates of bulk edited iPSC populations (S1 and S2) comprising mbIL-15 knock-in at the GAPDH gene were differentiated into iNK cells (day 39 of iPSC differentiation) and functioned to reduce tumor cell spheroid size when compared to WT parental cells also differentiated into iNK cells. Addition of 5 ng/mL exogenous IL-15 increased tumor cell killing by iNKs. The Y axis depicts normalized total integrated red object intensity, a proxy for tumor cell abundance, while the X axis depicts E:T ratio.

FIG. 37A shows the results of solid tumor killing assays as described in FIG. 26. Two biological replicates of bulk edited iPSC populations (S1 and S2) comprising mbIL-15 knock-in at the GAPDH gene were differentiated into iNK cells (day 63 of iPSC differentiation for S1, and day 56 of iPSC differentiation for S2) and functioned to reduce tumor cell spheroid size. The Y axis represents killing efficacy as measured by normalized total red object area (e.g., presence of tumor cells), while the X axis represents the E:T cell ratio; experiments were performed in duplicate or triplicate, R1, R2, and R2.1.

FIG. 37B shows the results of solid tumor killing assays as described in 37A, but with the addition of 10 μg/mL Herceptin antibody, an addition that triggers ADCC tumor cell killing.

FIG. 37C shows the results of solid tumor killing assays as described in 37A, but with the addition of 5 ng/mL exogenous IL-15.

FIG. 37D shows the results of solid tumor killing assays as described in 37A, but with the addition of 5 ng/mL exogenous IL-15 and 10 μg/mL Herceptin antibody, an addition that triggers ADCC tumor cell killing.

FIG. 38 depicts the cumulative results of two independent sets of cells and 3-5 repeats of solid tumor killing assays as described in FIG. 26. Two independent bulk edited populations (S1 and S2) comprising mbIL-15 knock-in at the GAPDH gene were differentiated into iNK cells (day 39 and 49 of iPSC differentiation for set 1, and day 42 of iPSC differentiation for S2) and functioned to significantly reduce tumor cell spheroid size when compared to differentiated WT parental cell iNKs in the absence of exogenous IL-15 (P=0.034, +/−standard deviation, unpaired t-test); in addition, differentiated knock-in cells trended towards significant reduction of tumor cell spheroid size when compared to differentiated WT parental cells in the presence of 5 ng/mL exogenous IL-15 (P=0.052, +/−standard deviation, unpaired t-test).

FIG. 39A schematically depicts a knock-in cassette cargo sequence comprising membrane-bound IL15.IL15Rα (mbIL-15) coupled with a GFP sequence, for integration at a target gene as described herein.

FIG. 39B schematically depicts a knock-in cassette cargo sequence comprising CD16, IL15, and IL15Rα, for integration at a target gene as described herein.

FIG. 39C schematically depicts a knock-in cassette cargo sequence comprising CD16 and membrane bound IL15.IL15Rα (mbIL-15), for integration at a target gene as described herein.

FIG. 40A depicts exemplary flow cytometry data from bulk edited iPSC populations seven days after transformation with PLA1829 (see FIG. 39A) comprising a cargo sequence of membrane-bound IL15.IL15Rα (mbIL-15) coupled with a GFP sequence inserted in the GAPDH gene using RNPs comprising RSQ22337 targeting the GAPDH gene and Cas12a (SEQ ID NO: 62), or control WT cells transformed with RNPs only, measured using ddPCR. Shown on the Y axis is IL-15Rα expression, while GFP expression is shown on the X axis.

FIG. 40B depicts exemplary flow cytometry data from bulk edited iPSC populations seven days after transformation with PLA1832 or PLA1834 (see FIGS. 39B and 39C), comprising a cargo sequence of CD16, IL-15, and IL15Rα, or comprising a cargo sequence of CD16 and membrane-bound IL15.IL15Rα (mbIL-15); inserted in the GAPDH gene using RNPs comprising RSQ22337 targeting the GAPDH gene and Cas12a (SEQ ID NO: 62), measured using ddPCR. Shown on the Y axis is IL-15Rα expression, X axis is GFP expression.

FIG. 41A is a histogram depicting the genotypes of individual colonies following transformation as described in FIG. 40A with PLA1829 (5 μg) and 2 μM RNPs comprising RSQ22337 targeting the GAPDH gene and Cas12a (SEQ ID NO: 62), measured using ddPCR. Shown are individual homozygous (˜100% TI), heterozygous (˜50% TI), or wild type (˜0% TI) cells.

FIG. 41B is a histogram depicting the genotypes of individual colonies following transformation as described in FIG. 40B with PLA1832 (5 μg) and 2 μM RNPs comprising RSQ22337 targeting the GAPDH gene and Cas12a (SEQ ID NO: 62), measured using ddPCR. Shown are individual homozygous (˜100% TI), heterozygous (˜50% TI), or wild type (˜0% TI) cells.

FIG. 41C is a histogram depicting the genotypes of individual colonies following transformation as described in FIG. 40B with PLA1834 (5 μg) and 2 μM RNPs comprising RSQ22337 targeting the GAPDH gene and Cas12a (SEQ ID NO: 62), measured using ddPCR. Shown are individual homozygous (˜100% TI), heterozygous (˜50% TI), or wild type (˜0% TI) cells.

FIG. 42A depicts exemplary flow cytometry data from cells comprising knock-in cargo sequences from PLA1829, PLA1832, or PLA1834 at the GAPDH gene (as described in FIG. 40A-40C) measured at day 32 of differentiation into iNKs; “WT” cells were transformed with RNPs only and were also at day 32 of differentiation into iNKs. The data highlights the efficiency of integration and expression of knock-in cassettes comprising an IL-15Rα protein encoding “cargo” sequence. The Y axis quantifies the percentage of cells from the noted population that are expressing IL-15Rα, while the X axis denotes colony genotype.

FIG. 42B depicts exemplary flow cytometry data from cells comprising knock-in cargo sequences from PLA1829, PLA1832, or PLA1834 at the GAPDH gene (as described in FIG. 40A-40C) measured at day 32 of differentiation into iNKs; “WT” cells were transformed with RNPs only and were also at day 32 of differentiation into iNKs. The data highlights the efficiency of integration and expression of knock-in cassettes comprising a CD16 protein encoding “cargo” sequence. The Y axis quantifies the percentage of cells from the noted population that are expressing CD16, while the X axis denotes colony genotype.

FIG. 42C depicts exemplary flow cytometry data from cells comprising knock-in cargo sequences from PLA1829, PLA1832, or PLA1834 at the GAPDH gene (as described in FIG. 40A-40C) measured at day 32 of differentiation into iNKs; “WT” cells were transformed with RNPs only and were also at day 32 of differentiation into iNKs. The data highlights the efficiency of integration and expression of knock-in cassettes comprising an IL-15Rα protein encoding “cargo” sequence. The Y axis quantifies the median fluorescence intensity (MFI) of a cell population expressing IL-15Rα, while the X axis denotes colony genotype.

FIG. 42D depicts exemplary flow cytometry data from cells comprising knock-in cargo sequences from PLA1829, PLA1832, or PLA1834 at the GAPDH gene (as described in FIG. 40A-40C) measured at day 32 of differentiation into iNKs; “WT” cells were transformed with RNPs only and were also at day 32 of differentiation into iNKs. The data highlights the efficiency of integration and expression of knock-in cassettes comprising a CD16 protein encoding “cargo” sequence. The Y axis quantifies the median fluorescence intensity (MFI) of a cell population expressing CD16, while the X axis denotes colony genotype.

FIG. 43A is a panel of cytometric dot plots showing further enrichment of PSCs that have been edited for a PDL1-based transgene, edited for a CD47-based transgene, or biallelically edited for a PDL1-based transgene and a CD47-based transgene targeted to the GAPDH gene locus, following a second round of editing with ribonucleoprotein (“RNP”) and PDL1-based and CD47-based donor constructs or RNP alone.

FIG. 43B is a panel of cytometric dot plots showing further enrichment of PSCs that have been edited for a PDL1-based transgene targeted to the GAPDH gene, following a second round of editing with RNP alone.

FIG. 44 depicts two cytometric dot plots showing unedited PSCs or enrichment of PSCs that have been edited at the GAPDH locus using two different donor templates, one of which is PDL1-based and the other is CD47-based. When editing using two different donor constructs, cells can be observed that are edited with either one unique donor construct (either PDL1-based or CD47-based) or biallelically edited for both a PDL1-based transgene and a CD47-based transgene targeted to the GAPDH gene.

### DETAILED DESCRIPTION

### Definitions and Abbreviations

Unless otherwise specified, each of the following terms have the meaning set forth in this section.

The indefinite articles “a” and “an” refer to at least one of the associated noun, and are used interchangeably with the terms “at least one” and “one or more.” The conjunctions “or” and “and/or” are used interchangeably as non-exclusive disjunctions.

The term “cancer” (also used interchangeably with the term “neoplastic”), as used herein, refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair.

The terms “CRISPR/Cas nuclease” as used herein refer to any CRISPR/Cas protein with DNA nuclease activity, e.g., a Cas9 or a Cas12 protein that exhibits specific association (or “targeting”) to a DNA target site, e.g., within a genomic sequence in a cell in the presence of a guide molecule. The strategies, systems, and methods disclosed herein can use any combination of CRISPR/Cas nuclease disclosed herein, or known to those of ordinary skill in the art. Those of ordinary skill in the art will be aware of additional CRISPR/Cas nucleases and variants suitable for use in the context of the present disclosure, and it will be understood that the present disclosure is not limited in this respect.

The term “differentiation” as used herein is the process by which an unspecialized (“uncommitted”) or less specialized cell acquires the features of a specialized cell such as, for example, a blood cell. In some embodiments, a differentiated or differentiation-induced cell is one that has taken on a more specialized (“committed”) position within the lineage of a cell. For example, an iPS cell (iPSC) can be differentiated into various more differentiated cell types, for example, a hematopoietic stem cell, a lymphocyte, and other cell types, upon treatment with suitable differentiation factors in the cell culture medium. Suitable methods, differentiation factors, and cell culture media for the differentiation of pluri- and multipotent cell types into more differentiated cell types are well known to those of skill in the art. In some embodiments, the term “committed”, is applied to the process of differentiation to refer to a cell that has proceeded through a differentiation pathway to a point where, under normal circumstances, it would or will continue to differentiate into a specific cell type or subset of cell types, and cannot, under normal circumstances, differentiate into a different cell type (other than a specific cell type or subset of cell types) nor revert to a less differentiated cell type.

The terms “differentiation marker,” “differentiation marker gene,” or “differentiation gene,” as used herein refers to genes or proteins whose expression are indicative of cell differentiation occurring within a cell, such as a pluripotent cell. In some embodiments, differentiation marker genes include, but are not limited to, the following genes: CD34, CD4, CD8, CD3, CD56 (NCAM), CD49, CD45, NK cell receptor (cluster of differentiation 16 (CD16)), natural killer group-2 member D (NKG2D), CD69, NKp30, NKp44, NKp46, CD158b, FOXA2, FGF5, SOX17, XIST, NODAL, COL3A1, OTX2, DUSP6, EOMES, NR2F2, NROB1, CXCR4, CYP2B6, GAT A3, GATA4, ERBB4, GATA6, HOXC6, INHA, SMAD6, RORA, NIPBL, TNFSF11, CDH11, ZIC4, GAL, SOX3, PITX2, APOA2, CXCL5, CER1, FOXQ1, MLL5, DPP10, GSC, PCDH10, CTCFL, PCDH20, TSHZ1, MEGF10, MYC, DKK1, BMP2, LEFTY2, HES1, CDX2, GNAS, EGR1, COL3A1, TCF4, HEPH, KDR, TOX, FOXA1, LCK, PCDH7, CD1D FOXG1, LEFTY1, TUJ1, T gene (Brachyury), ZIC1, GATA1, GATA2, HDAC4, HDAC5, HDAC7, HDAC9, NOTCH1, NOTCH2, NOTCH4, PAX5, RBPJ, RUNX1, STAT1 and STAT3.

The terms “differentiation marker gene profile,” or “differentiation gene profile,” “differentiation gene expression profile,” “differentiation gene expression signature,” “differentiation gene expression panel,” “differentiation gene panel,” or “differentiation gene signature” as used herein refer to expression or levels of expression of a plurality of differentiation marker genes.

The term “nuclease” as used herein refers to any protein that catalyzes the cleavage of phosphodiester bonds. In some embodiments the nuclease is a DNA nuclease. In some embodiments the nuclease is a “nickase” which causes a single-strand break when it cleaves double-stranded DNA, e.g., genomic DNA in a cell. In some embodiments the nuclease causes a double-strand break when it cleaves double-stranded DNA, e.g., genomic DNA in a cell. In some embodiments the nuclease binds a specific target site within the double-stranded DNA that overlaps with or is adjacent to the location of the resulting break. In some embodiments, the nuclease causes a double-strand break that contains overhangs ranging from 0 (blunt ends) to 22 nucleotides in both 3′ and 5′ orientations. As discussed herein, CRISPR/Cas nucleases, zinc finger nucleases (ZFNs), transcription activator-like effector nucleases (TALENs) and meganucleases are exemplary nucleases that can be used in accordance with the strategies, systems, and methods of the present disclosure.

The term “embryonic stem cell” as used herein refers to pluripotent stem cells derived from the inner cell mass of the embryonic blastocyst. In some embodiments, embryonic stem cells are pluripotent and give rise during development to all derivatives of the three primary germ layers: ectoderm, endoderm and mesoderm. In some such embodiments, embryonic stem cells do not contribute to the extra-embryonic membranes or the placenta, i.e., are not totipotent.

The term “endogenous,” as used herein in the context of nucleic acids refers to a native nucleic acid (e.g., a gene, a protein coding sequence) in its natural location, e.g., within the genome of a cell.

The term “essential gene” as used herein with respect to a cell refers to a gene that encodes at least one gene product that is required for survival and/or proliferation of the cell. An essential gene can be a housekeeping gene that is essential for survival of all cell types or a gene that is required to be expressed in a specific cell type for survival and/or proliferation under particular culture conditions, e.g., for proper differentiation of iPS or ES cells or expansion of iPS- or ES-derived cells. Loss of function of an essential gene results, in some embodiments, in a significant reduction of cell survival, e.g., of the time a cell characterized by a loss of function of an essential gene survives as compared to a cell of the same cell type but without a loss of function of the same essential gene. In some embodiments, loss of function of an essential gene results in the death of the affected cell. In some embodiments, loss of function of an essential gene results in a significant reduction of cell proliferation, e.g., in the ability of a cell to divide, which can manifest in a significant time period the cell requires to complete a cell cycle, or, in some preferred embodiments, in a loss of a cell's ability to complete a cell cycle, and thus to proliferate at all.

The term “exogenous,” as used herein in the context of nucleic acids refers to a nucleic acid (whether native or non-native) that has been artificially introduced into a man-made construct (e.g., a knock-in cassette, or a donor template) or into the genome of a cell using, for example, gene editing or genetic engineering techniques, e.g., HDR based integration techniques.

The term “guide molecule” or “guide RNA” or “gRNA” when used in reference to a CRISPR/Cas system is any nucleic acid that promotes the specific association (or “targeting”) of a CRISPR/Cas nuclease, e.g., a Cas9 or a Cas12 protein to a DNA target site such as within a genomic sequence in a cell. While guide molecules are typically RNA molecules it is well known in the art that chemically modified RNA molecules including DNA/RNA hybrid molecules can be used as guide molecules.

The terms “hematopoietic stem cell,” or “definitive hematopoietic stem cell” as used herein, refer to CD34-positive (CD34+) stem cells. In some embodiments, CD34-positive stem cells are capable of giving rise to mature myeloid and/or lymphoid cell types. In some embodiments, the myeloid and/or lymphoid cell types include, for example, T cells, natural killer (NK) cells and/or B cells.

The terms “induced pluripotent stem cell”, “iPS cell” or “iPSC” as used herein to refer to a stem cell obtained from a differentiated somatic (e.g., adult, neonatal, or fetal) cell by a process referred to as reprogramming (e.g., dedifferentiation). In some embodiments, reprogrammed cells are capable of differentiating into tissues of all three germ or dermal layers: mesoderm, endoderm, and ectoderm. iPSCs are not found in nature.

The terms “iPS-derived NK cell” or “iNK cell” or as used herein refers to a natural killer cell which has been produced by differentiating an iPS cell, which iPS cell may or may not have a genetic modification.

The terms “iPS-derived T cell” or “iT cell” or as used herein refers to a T which has been produced by differentiating an iPS cell, which iPS cell may or may not have a genetic modification.

The term “multipotent stem cell” as used herein refers to a cell that has the developmental potential to differentiate into cells of one or more germ layers (ectoderm, mesoderm and endoderm), but not all three germ layers. Thus, in some embodiments, a multipotent cell may also be termed a “partially differentiated cell.” Multipotent cells are well-known in the art, and examples of multipotent cells include adult stem cells, such as for example, hematopoietic stem cells and neural stem cells. In some embodiments, “multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, in some embodiments, “multipotency” refers to a state of a cell with a degree of developmental potential that is less than totipotent and pluripotent.

The term “pluripotent” as used herein refers to ability of a cell to form all lineages of the body or soma (i.e., the embryo proper) or a given organism (e.g., human). For example, embryonic stem cells are a type of pluripotent stem cells that are able to form cells from each of the three germs layers, the ectoderm, the mesoderm, and the endoderm. Generally, pluripotency may be described as a continuum of developmental potencies ranging from an incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell or an induced pluripotent stem cell).

The term “pluripotency” as used herein refers to a cell that has the developmental potential to differentiate into cells of all three germ layers (ectoderm, mesoderm, and endoderm). In some embodiments, pluripotency can be determined, in part, by assessing pluripotency characteristics of the cells. In some embodiments, pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4 (also known as POU5F1), NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

The term “pluripotent stem cell morphology” as used herein refers to the classical morphological features of an embryonic stem cell. In some embodiments, normal embryonic stem cell morphology is characterized as small and round in shape, with a high nucleus-to-cytoplasm ratio, the notable presence of nucleoli, and typical intercell spacing.

The term “polycistronic” or “multicistronic” when used herein with reference to a knock-in cassette refers to the fact that the knock-in cassette can express two or more proteins from the same mRNA transcript. Similarly, a “bicistronic” knock-in cassette is a knock-in cassette that can express two proteins from the same mRNA transcript.

The term “polynucleotide” (including, but not limited to “nucleotide sequence”, “nucleic acid”, “nucleic acid molecule”, “nucleic acid sequence”, and “oligonucleotide”) as used herein refer to a series of nucleotide bases (also called “nucleotides”) in DNA and RNA, and mean any chain of two or more nucleotides. In some embodiments, polynucleotides, nucleotide sequences, nucleic acids, etc. can be chimeric mixtures or derivatives or modified versions thereof, single-stranded or double-stranded. In some such embodiments, modifications can occur at the base moiety, sugar moiety, or phosphate backbone, for example, to improve stability of the molecule, its hybridization parameters, etc. In general, a nucleotide sequence typically carries genetic information, including, but not limited to, the information used by cellular machinery to make proteins and enzymes. In some embodiments, a nucleotide sequence and/or genetic information comprises double- or single-stranded genomic DNA, RNA, any synthetic and genetically manipulated polynucleotide, and/or sense and/or antisense polynucleotides. In some embodiments, nucleic acids containing modified bases.

Conventional IUPAC notation is used in nucleotide sequences presented herein, as shown in Table 1, below (see also Cornish-Bowden, Nucleic Acids Res. 1985; 13(9):3021-30, incorporated by reference herein). It should be noted, however, that “T” denotes “Thymine or Uracil” in those instances where a sequence may be encoded by either DNA or RNA, for example in certain CRISPR/Cas guide molecule targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “potency” or “developmental potency” as used herein refer to the sum of all developmental options accessible to the cell (i.e., the developmental potency), particularly, for example in the context of cellular developmental potential. In some embodiments, the continuum of cell potency includes, but is not limited to, totipotent cells, pluripotent cells, multipotent cells, oligopotent cells, unipotent cells, and terminally differentiated cells.

The terms “prevent,” “preventing,” and “prevention” as used herein refer to the prevention of a disease in a mammal, e.g., in a human, including (a) avoiding or precluding the disease; (b) affecting the predisposition toward the disease; or (c) preventing or delaying the onset of at least one symptom of the disease.

The terms “protein,” “peptide” and “polypeptide” as used herein are used interchangeably to refer to a sequential chain of amino acids linked together via peptide bonds. The terms include individual proteins, groups or complexes of proteins that associate together, as well as fragments or portions, variants, derivatives and analogs of such proteins. Unless otherwise specified, peptide sequences are presented herein using conventional notation, beginning with the amino or N-terminus on the left, and proceeding to the carboxyl or C-terminus on the right. Standard one-letter or three-letter abbreviations can be used.

The term “gene product of interest” as used herein can refer to any product encoded by a gene including any polynucleotide or polypeptide. In some embodiments the gene product is a protein which is not naturally expressed by a target cell of the present disclosure. In some embodiments the gene product is a protein which confers a new therapeutic activity to the cell such as, but not limited to, a chimeric antigen receptor (CAR) or antigen-binding fragment thereof, a T cell receptor or antigen-binding portion thereof, a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof. It is to be understood that the methods and cells of the present disclosure are not limited to any particular gene product of interest and that the selection of a gene product of interest will depend on the type of cell and ultimate use of the cells.

The term “reporter gene” as used herein refers to an exogenous gene that has been introduced into a cell, e.g., integrated into the genome of the cell, that confers a trait suitable for artificial selection. Common reporter genes are fluorescent reporter genes that encode a fluorescent protein, e.g., green fluorescent protein (GFP) and antibiotic resistance genes that confer antibiotic resistance to cells.

The terms “reprogramming” or “dedifferentiation” or “increasing cell potency” or “increasing developmental potency” as used herein refer to a method of increasing potency of a cell or dedifferentiating a cell to a less differentiated state. For example, in some embodiments, a cell that has an increased cell potency has more developmental plasticity (i.e., can differentiate into more cell types) compared to the same cell in the non-reprogrammed state. That is, in some embodiments, a reprogrammed cell is one that is in a less differentiated state than the same cell in a non-reprogrammed state. In some embodiments, “reprogramming” refers to de-differentiating a somatic cell, or a multipotent stem cell, into a pluripotent stem cell, also referred to as an induced pluripotent stem cell, or iPSC. Suitable methods for the generation of iPSCs from somatic or multipotent stem cells are well known to those of skill in the art.

The term “subject” as used herein means a human or non-human animal. In some embodiments a human subject can be any age (e.g., a fetus, infant, child, young adult, or adult). In some embodiments a human subject may be at risk of or suffer from a disease, or may be in need of alteration of a gene or a combination of specific genes. Alternatively, in some embodiments, a subject may be a non-human animal, which may include, but is not limited to, a mammal. In some embodiments, a non-human animal is a non-human primate, a rodent (e.g., a mouse, rat, hamster, guinea pig, etc.), a rabbit, a dog, a cat, and so on. In certain embodiments of this disclosure, the non-human animal subject is livestock, e.g., a cow, a horse, a sheep, a goat, etc. In certain embodiments, the non-human animal subject is poultry, e.g., a chicken, a turkey, a duck, etc.

The terms “treatment,” “treat,” and “treating,” as used herein refer to a clinical intervention aimed to reverse, alleviate, delay the onset of, or inhibit the progress, ameliorate, reduce severity of, prevent or delay the recurrence of a disease, disorder, or condition or one or more symptoms thereof, and/or improve one or more symptoms of a disease, disorder, or condition as described herein. In some embodiments, a condition includes an injury. In some embodiments, an injury may be acute or chronic (e.g., tissue damage from an underlying disease or disorder that causes, e.g., secondary damage such as tissue injury). In some embodiments, treatment, e.g., in the form of an iPSC-derived NK cell or a population of iPSC-derived NK cells as described herein, may be administered to a subject after one or more symptoms have developed and/or after a disease has been diagnosed. Treatment may be administered in the absence of symptoms, e.g., to prevent or delay onset of a symptom or inhibit onset or progression of a disease. For example, in some embodiments, treatment may be administered to a susceptible subject prior to the onset of symptoms (e.g., in light of genetic or other susceptibility factors). In some embodiments, treatment may also be continued after symptoms have resolved, for example to prevent or delay their recurrence. In some embodiments, treatment results in improvement and/or resolution of one or more symptoms of a disease, disorder or condition.

The term “variant” as used herein refers to an entity such as a polypeptide or polynucleotide that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As used herein, the terms “functional variant” refer to a variant that confers the same function as the reference entity, e.g., a functional variant of a gene product of an essential gene is a variant that promotes the survival and/or proliferation of a cell. It is to be understood that a functional variant need not be functionally equivalent to the reference entity as long as it confers the same function as the reference entity.

### Methods of Editing the Genome of a Cell

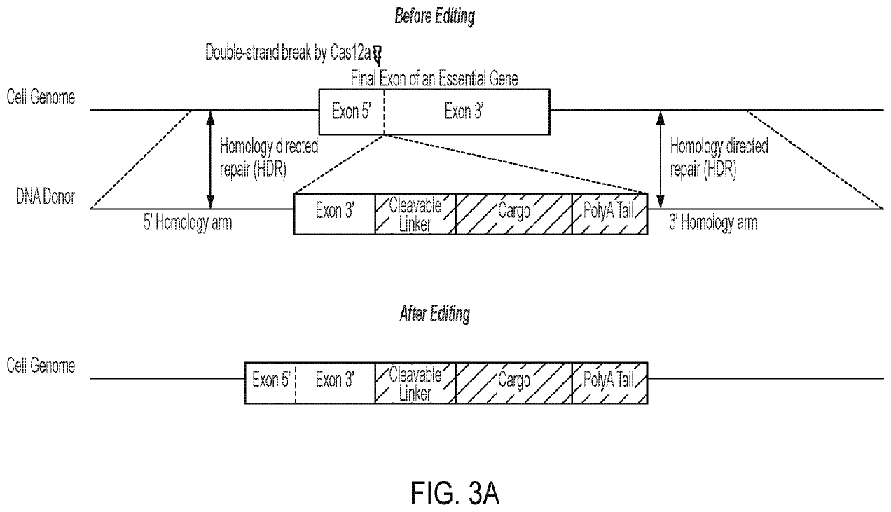

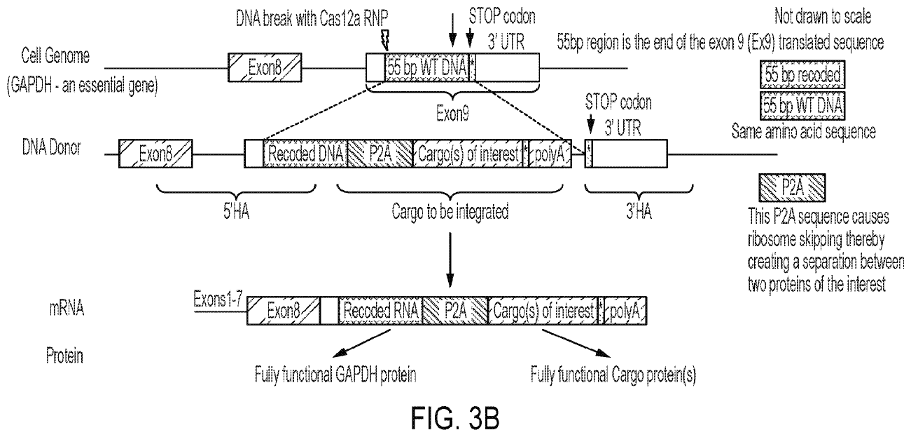

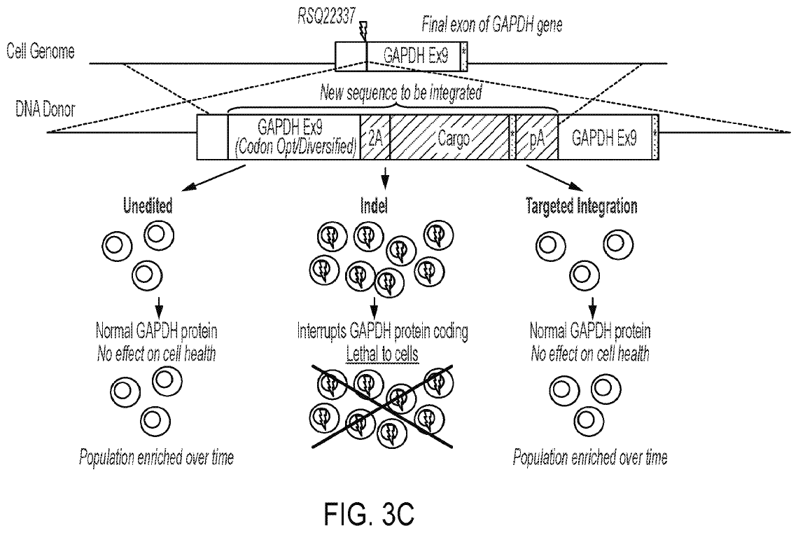

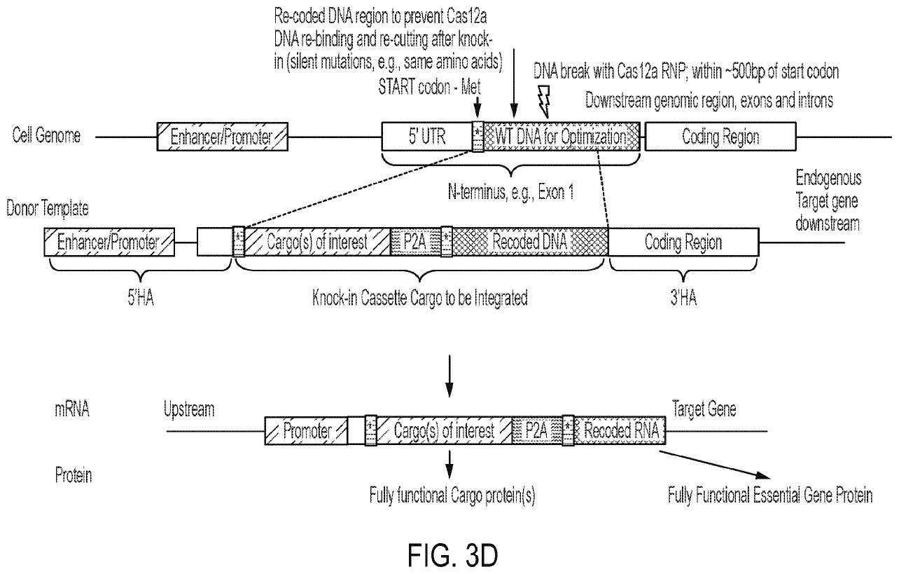

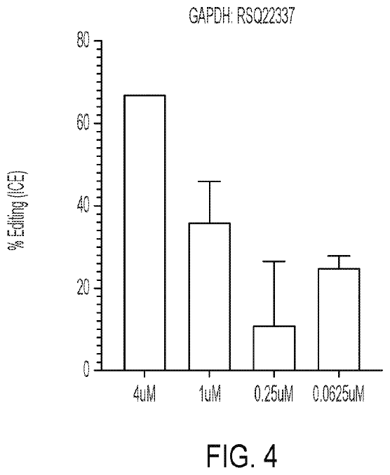

In one aspect, the present disclosure provides methods of editing the genome of a cell. In certain embodiments, the method comprises contacting the cell with a nuclease that causes a break within an endogenous coding sequence of an essential gene in the cell wherein the essential gene encodes at least one gene product that is required for survival and/or proliferation of the cell. The cell is also contacted with (i) a donor template that comprises a knock-in cassette comprising an exogenous coding sequence for a gene product of interest in frame with and downstream (3′) of an exogenous coding sequence or partial coding sequence of the essential gene and/or (ii) a donor template that comprises a knock-in cassette comprising an exogenous coding sequence for a gene product of interest in frame with and upstream (5′) of an exogenous coding sequence or partial coding sequence of the essential gene (FIG. 3D). The knock-in cassette is integrated into the genome of the cell by homology-directed repair (HDR) of the break, resulting in a genome-edited cell that expresses the gene product of interest and the gene product encoded by the essential gene that is required for survival and/or proliferation of the cell, or a functional variant thereof. The genetically modified “knock-in” cell survives and proliferates to produce progeny cells with genomes that also include the exogenous coding sequence for the gene product of interest. This is illustrated in FIG. 3A for an exemplary method.

If the knock-in cassette is not properly integrated into the genome of the cell, undesired editing events that result from the break, e.g., NHEJ-mediated creation of indels, may produce a non-functional, e.g., out of frame, version of the essential gene. This produces a “knock-out” cell when the editing efficiency of the nuclease is high enough to disrupt both alleles. In certain embodiments, this produces a “knock-out” cell when the editing efficiency of the nuclease is high enough to disrupt one allele. Without sufficient functional copies of the essential gene these “knock-out” cells are unable to survive and do not produce any progeny cells.

Since the “knock-in” cells survive and the “knock-out” cells do not survive, the method automatically selects for the “knock-in” cells when it is applied to a population of starting cells. Significantly, in certain embodiments, the method does not require high knock-in efficiencies because of this automatic selection aspect. It is therefore particularly suitable for methods where the donor template is a dsDNA (e.g., a plasmid) where knock-in efficiencies are often below 5%. As noted in the exemplary method of FIG. 3C, in some embodiments some of the cells in the population of starting cells may remain unedited, i.e., unaffected by the nuclease. These cells would also survive and produce progeny with genomes that do not include the exogenous coding sequence for the gene product of interest. When the nuclease editing efficiency is high, e.g., about 60-90%, or higher the percentage of unedited cells will be relatively low as compared to the percentage of genetically modified cells. In some embodiments, high nuclease editing efficiencies (e.g., greater than 65%, greater than 70%, greater than 75%, greater than 80%, greater than 85%, greater than 90%, or greater than 95%) facilitates efficient population wide transgene integration, as the percentage of unedited cells will be relatively low as compared to the percentage of genetically modified cells. In some embodiments of the methods disclosed herein, at least about 65% of the cells (e.g., about 70%, about 75%, about 80%, about 85%, about 90%, about 91%, about 92%, about 93%, about 94%, about 95%, about 96%, about 97%, about 98%, or about 99% of the cells) are edited by a nuclease, e.g., an Cas12a or Cas9. In some embodiments, an RNP containing a CRISPR nuclease (e.g., Cas9 or Cas12a) and a guide are capable of cleaving the locus of an essential gene (e.g., a terminal exon in the locus of any essential gene provided in Table 3) in at least 65% of the cells in a population of cells (e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the cells in a population of cells). In some embodiments, editing efficiency is determined prior to target cell die off, e.g., at day 1 and/or day 2 post transfection or transduction. In some embodiments, editing efficiency measured at day 1 and/or day 2 post transfection or transduction may not capture the complete proportion of cells for which editing occurred, as in some embodiments, certain editing events may result in near immediate and/or swift cell death. In some embodiments, near immediate and/or swift cell death may be any period of time less than 48 hours post transfection or transduction, for example, less than 48 hours, less than 44 hours, less than 40 hours, less than 36 hours, less than 32 hours, less than 28 hours, less than 24 hours, less than 20 hours, less than 16 hours, less than 15 hours, less than 14 hours, less than 13 hours, less than 12 hours, less than 11 hours, less than 10 hours, less than 9 hours, less than 8 hours, less than 7 hours, less than 6 hours, less than 5 hours, less than 4 hours, less than 3 hours, less than 2 hours, or less than 1 hour after transfection or transduction.

In some embodiments, the nuclease causes a double-strand break. In some embodiments the nuclease causes a single-strand break, e.g., in some embodiments the nuclease is a nickase. In some embodiments the nuclease is a prime editor which comprises a nickase domain fused to a reverse transcriptase domain. In some embodiments the nuclease is an RNA-guided prime editor and the gRNA comprises the donor template. In some embodiments a dual-nickase system is used which causes a double-strand break via two single-strand breaks on opposing strands of a double-stranded DNA, e.g., genomic DNA of the cell.

In some embodiments, the present disclosure provides methods suitable for high-efficiency knock-in (e.g., a high proportion of a cell population comprises a knock-in allele), overcoming a major manufacturing challenge. Historically, gene of interest knock-in using plasmid vectors results in efficiencies typically between 0.1 and 5% (see e.g., Zhu et al., CRISPR/Cas-Mediated Selection-free Knockin Strategy in Human Embryonic Stem Cells. Stem Cell Reports. 2015; 4(6):1103-1111), this low knock-in efficiency can result in a need for extensive time and resources devoted to screening potentially edited clones.

In some embodiments, a gene of interest knocked into a cell may have a role in effector function, specificity, stealth, persistence, homing/chemotaxis, and/or resistance to certain chemicals (see for example, Saetersmoen et al., Seminars in Immunopathology, 2019).

In certain embodiments, the present disclosure provides methods for creation of knock-in cells that maintain high levels of expression regardless of age, differentiation status, and/or exogenous conditions. For example, in some embodiments, an integrated cargo is expressed at an optimal level with a desired subcellular localization as a function of an insertion site. In some embodiments, the present disclosure provides such cells.

### Systems for Editing the Genome of a Cell

In one aspect the present disclosure provides systems for editing the genome of a cell. In some embodiments, the system comprises the cell, a nuclease that causes a break within an endogenous coding sequence of an essential gene of the cell, wherein the essential gene encodes a gene product that is required for survival and/or proliferation of the cell, and a donor template that comprises a knock-in cassette comprising an exogenous coding sequence for a gene product of interest in frame with and downstream (3′) of an exogenous coding sequence or partial coding sequence of the essential gene.

In some embodiments, the nuclease causes a double-strand break. In some embodiments the nuclease causes a single-strand break, e.g., in some embodiments the nuclease is a nickase. In some embodiments the nuclease is a prime editor which comprises a nickase domain fused to a reverse transcriptase domain. In some embodiments the nuclease is an RNA-guided prime editor and the gRNA comprises the donor template. In some embodiments a dual-nickase system is used which causes a double-strand break via two single-strand breaks on opposing strand of a double-stranded DNA, e.g., genomic DNA of the cell.

Genome editing systems can be implemented (e.g., administered or delivered to a cell or a subject) in a variety of ways, and different implementations may be suitable for distinct applications. For instance, a genome editing system is implemented, in certain embodiments, as a protein/RNA complex (a ribonucleoprotein, or RNP). In certain embodiments, a genome editing system is implemented as one or more nucleic acids encoding an RNA-guided nuclease and guide RNA components described herein (optionally with one or more additional components); in certain embodiments, a genome editing system is implemented as one or more vectors comprising such nucleic acids, for instance a viral vector such as an adeno-associated virus; and in certain embodiments, a genome editing system is implemented as a combination of any of the foregoing. Additional or modified implementations that operate according to the principles set forth herein will be apparent to the skilled artisan and are within the scope of this disclosure.

In some embodiments, methods as described herein include performing certain steps in at least duplicate. For example, in some embodiments, integration of certain gene products of interest, particularly including multiple genes of interest or a large number of exogenous gene sequences, may result in an initial selection round that results in a lower than desired level of targeted integration. In certain embodiments, a lower than desirable levels of nuclease activity and/or of knock-in cassette targeted integration may result in a lower than desirable percentage of surviving cells and/or cells comprising the knock-in cassette; this may make identifying a cell with the genetic payload difficult. In some embodiments, to further enrich for the population of edited cells, cells were optionally expanded and then re-edited by providing the pool of edited cells with either both RNP and donor templates (e.g., one or more RNP particles targeting one or more loci, and one or more donor templates designed for targeted integration at one or more loci), or just RNP alone (e.g., one or more RNP that utilize residual donor template).

In some embodiments, where multiple rounds of RNP and/or donor template editing is performed, enrichment is affected by: i) removing cells that have not incorporated the genetic payload and/or ii) creating more cells with incorporated knock-in cassette. In some embodiments, the effectiveness of an additional enrichment steps, depending on the cargo, depending on whether multiple constructs are used, the target within the essential gene, or other factors, can lead to at least about two-fold, three-fold, four-fold, five-fold, or higher improvement in the percentage of cells incorporating the knock-in cassette from the donor template. In some embodiments, such enrichment can lead to uptake of the “cargo” within the essential gene of mammalian cells of greater than 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or greater than 95%.

In some embodiments, donor templates (e.g., donor nucleic acid constructs) comprise the transgene flanked by a first homologous region (HR) e.g., a homology arm, and a second HR, e.g., a second homology arm, designed to anneal to a first genomic region (GR) and a second GR within an essential gene of a cell. To be able to anneal, the HRs and GRs need not be perfectly homologous. In some embodiments, examples include a non-inhibitory small number (less than 6 and as few as 1) of mutations in the PAM 5′ of the transgene in the knock-in cassette. In some embodiments, other non-inhibitory changes include codon optimization, wherein unnecessary nucleotides in the wildtype exon are removed from the nucleotide sequence in the knock-in cassette. In some embodiments, other such silent PAM blocking mutations or a codon modifications that prevents cleavage of the donor nucleic acid construct by the nuclease are further contemplated. In some embodiments, at least about 90% homology is sufficient for functional annealing for purposes of the examples herein. In some embodiments, the level of homology between the HR and GR is more than 90%, more than 92%, more than 94%, more than 96%, more than 98%, or more than 99%. Other embodiments and the concepts set forth in this paragraph are contemplated and subsumed in the term “essentially homologous.”

### Genetically Modified Cells

In one aspect the present disclosure provides genetically modified cells or engineered cells including populations of such cells and progeny of such cells.

In some embodiments, the cell is produced by a method of the present disclosure, e.g., a method that comprises contacting the cell with a nuclease that causes a break within an endogenous coding sequence of an essential gene in the cell wherein the essential gene encodes at least one gene product that is required for survival and/or proliferation of the cell. The cell is also contacted with a donor template that comprises a knock-in cassette comprising an exogenous coding sequence for a gene product of interest in frame with and downstream (3′) of an exogenous coding sequence or partial coding sequence of the essential gene. The knock-in cassette is integrated into the genome of the cell by homology-directed repair (HDR) of the break, resulting in a genome-edited cell that expresses the gene product of interest and the gene product encoded by the essential gene that is required for survival and/or proliferation of the cell, or a functional variant thereof. This is illustrated in FIG. 3 for an exemplary method. In some embodiments, a cell is contacted with a donor template that comprises a knock-in cassette comprising an exogenous coding sequence for a gene product of interest in frame with and upstream (5′) of an exogenous coding sequence or partial coding sequence of the essential gene.

In some embodiments, the cell comprises a genome with an exogenous coding sequence for a gene product of interest in frame with and downstream (3′) of a coding sequence of an essential gene, wherein the essential gene encodes a gene product that is required for survival and/or proliferation of the cell.

In some embodiments, the cell comprises a genome with an exogenous coding sequence for a gene product of interest in frame with and upstream (5′) of a coding sequence of an essential gene, wherein the essential gene encodes a gene product that is required for survival and/or proliferation of the cell.

In some embodiments, the cell comprises a genomic modification, wherein the genomic modification comprises an insertion of an exogenous knock-in cassette within an endogenous coding sequence of an essential gene in the cell's genome, wherein the essential gene encodes a gene product that is required for survival and/or proliferation of the cell, wherein the knock-in cassette comprises an exogenous coding sequence for a gene product of interest in frame with and downstream (3′) of an exogenous coding sequence or partial coding sequence encoding the gene product of the essential gene, or a functional variant thereof, and wherein the cell expresses the gene product of interest and the gene product encoded by the essential gene that is required for survival and/or proliferation of the cell, or a functional variant thereof. In some embodiments, the gene product of interest and the gene product encoded by the essential gene are expressed from the endogenous promoter of the essential gene.

### Donor Template

In one aspect the present disclosure provides a donor template comprising a knock-in cassette with an exogenous coding sequence for a gene product of interest in frame with and downstream (3′) of an exogenous coding sequence or partial coding sequence of an essential gene, wherein the essential gene encodes a gene product that is required for survival and/or proliferation of the cell.

In one aspect the present disclosure provides an impetus for designing donor templates comprising a knock-in cassette with an exogenous coding sequence for a gene product of interest in frame with and upstream (5′) of an exogenous coding sequence or partial coding sequence of an essential gene, wherein the essential gene encodes a gene product that is required for survival and/or proliferation of the cell; see e.g., FIG. 3D.

In some embodiments, the donor template is for use in editing the genome of a cell by homology-directed repair (HDR).

Donor template design is described in detail in the literature, for instance in PCT Publication No. WO2016/073990A1. Donor templates can be single-stranded or double-stranded and can be used to facilitate HDR-based repair of double-strand breaks (DSBs), and are particularly useful for inserting a new sequence into the target sequence, or replacing the target sequence altogether. In some embodiments, the donor template is a donor DNA template. In some embodiments the donor DNA template is double-stranded.

Whether single-stranded or double stranded, donor templates generally include regions that are homologous to regions of DNA within or near (e.g., flanking or adjoining) a target sequence to be cleaved. These homologous regions are referred to herein as “homology arms,” and are illustrated schematically below relative to the knock-in cassette (which may be separated from one or both of the homology arms by additional spacer sequences that are not shown):

[5′ homology arm]—[knock-in cassette]—[3′ homology arm].

The homology arms can have any suitable length (including 0 nucleotides if only one homology arm is used), and 5′ and 3′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements.

In some embodiments, more than one donor template can be administered to a cell population. In some embodiments, the more than one donor templates are different, for example, each donor template facilitates knock-in of “cargo” sequences encoding different gene products of interest. In some embodiments, the more than one donor templates can be provided at the same time and their payloads incorporated into the same essential gene (e.g., one incorporated at one allele, the other incorporated at the other allele). In some embodiments, this may be particularly advantageous when a particular transgene system and/or gene product of interest has functional sequences that require them to be separated into different alleles of an essential gene. Further, in some embodiments, having multiple copies of gene targets of interest that are different but accomplish a similar goal, e.g., copies of safety switches, can be helpful to assure the functionality and creation of a corresponding phenotype. In some embodiments, more than one copy of a safety switch can ensure elimination of cells when necessary. Further, in some embodiments, certain safety switches requires dimerization to function as a suicide switch system (e.g., as described herein). In some embodiments, when more than one donor template is administered to a cell population, such donor templates may be designed to integrate at the same genetic locus, or at different genetic loci.

A donor template can be a nucleic acid vector, such as a viral genome or circular double-stranded DNA, e.g., a plasmid. Nucleic acid vectors comprising donor templates can include other coding or non-coding elements. For example, a donor template nucleic acid can be delivered as part of a viral genome (e.g., in an AAV, adenoviral, Sendai virus, or lentiviral genome) that includes certain genomic backbone elements (e.g., inverted terminal repeats, in the case of an AAV genome). In some embodiments, a donor template is comprised in a plasmid that has not been linearized. In some embodiments, a donor template is comprised in a plasmid that has been linearized. In some embodiments, a donor template is comprised within a linear dsDNA fragment. In some embodiments, a donor template nucleic acid can be delivered as part of an AAV genome. In some embodiments, a donor template nucleic acid can be delivered as a single stranded oligo donor (ssODN), for example, as a long multi-kb ssODN derived from m13 phage synthesis, or alternatively, short ssODNs, e.g., that comprise small genes of interest, tags, and/or probes. In some embodiments, a donor template nucleic acid can be delivered as a Doggybone™ DNA (dbDNA™) template. In some embodiments, a donor template nucleic acid can be delivered as a DNA minicircle. In some embodiments, a donor template nucleic acid can be delivered as a Integration-deficient Lentiviral Particle (IDLV). In some embodiments, a donor template nucleic acid can be delivered as a MMLV-derived retrovirus. In some embodiments, a donor template nucleic acid can be delivered as a piggyBac™ sequence. In some embodiments, a donor template nucleic acid can be delivered as a replicating EBNA1 episome.

In certain embodiments, the 5′ homology arm may be about 25 to about 1,000 base pairs in length, e.g., at least about 100, 200, 400, 600, or 800 base pairs in length. In certain embodiments, the 5′ homology arm comprises about 50 to 800 base pairs, e.g., 100 to 800, 200 to 800, 400 to 800, 400 to 600, or 600 to 800 base pairs. In certain embodiments, the 3′ homology arm may be about 25 to about 1,000 base pairs in length, e.g., at least about 100, 200, 400, 600, or 800 base pairs in length. In certain embodiments, the 3′ homology arm comprises about 50 to 800 base pairs, e.g., 100 to 800, 200 to 800, 400 to 800, 400 to 600, or 600 to 800 base pairs. In certain embodiments, the 5′ and 3′ homology arms are symmetrical in length. In certain embodiments, the 5′ and 3′ homology arms are asymmetrical in length.

In certain embodiments, a 5′ homology arm is less than about 3,000 base pairs, less than about 2,900 base pairs, less than about 2,800 base pairs, less than about 2,700 base pairs, less than about 2,600 base pairs, less than about 2,500 base pairs, less than about 2,400 base pairs, less than about 2,300 base pairs, less than about 2,200 base pairs, less than about 2,100 base pairs, less than about 2,000 base pairs, less than about 1,900 base pairs, less than about 1,800 base pairs, less than about 1,700 base pairs, less than about 1,600 base pairs, less than about 1,500 base pairs, less than about 1,400 base pairs, less than about 1,300 base pairs, less than about 1,200 base pairs, less than about 1,100 base pairs, less than about 1,000 base pairs, less than about 900 base pairs, less than about 800 base pairs, less than about 700 base pairs, less than about 600 base pairs, less than about 500 base pairs, or less than about 400 base pairs.

In certain embodiments, e.g., where a viral vector is utilized to introduce a knock-in cassette through a method described herein, a 5′ homology arm is less than about 1,000 base pairs, less than about 900 base pairs, less than about 800 base pairs, is less than about 700 base pairs, less than about 600 base pairs, less than about 500 base pairs, less than about 400 base pairs, or less than about 300 base pairs. In certain embodiments, e.g., where a viral vector is utilized to introduce a knock-in cassette through a method described herein, a 5′ homology arm is about 400-600 base pairs, e.g., about 500 base pairs.

In certain embodiments, a 3′ homology arm is less than about 3,000 base pairs, less than about 2,900 base pairs, less than about 2,800 base pairs, less than about 2,700 base pairs, less than about 2,600 base pairs, less than about 2,500 base pairs, less than about 2,400 base pairs, less than about 2,300 base pairs, less than about 2,200 base pairs, less than about 2,100 base pairs, less than about 2,000 base pairs, less than about 1,900 base pairs, less than about 1,800 base pairs, less than about 1,700 base pairs, less than about 1,600 base pairs, less than about 1,500 base pairs, less than about 1,400 base pairs, less than about 1,300 base pairs, less than about 1,200 base pairs, less than about 1,100 base pairs, less than 1,000 base pairs, less than about 900 base pairs, less than about 800 base pairs, less than about 700 base pairs, less than about 600 base pairs, less than about 500 base pairs, or less than about 400 base pairs.

In certain embodiments, e.g., where a viral vector is utilized to introduce a knock-in cassette through a method described herein, a 3′ homology arm is less than about 1,000 base pairs, less than about 900 base pairs, less than about 800 base pairs, less than about 700 base pairs, less than about 600 base pairs, less than about 500 base pairs, less than about 400 base pairs, or less than about 300 base pairs. In certain embodiments, e.g., where a viral vector is utilized to introduce a knock-in cassette through a method described herein, a 3′ homology arm is about 400-600 base pairs, e.g., about 500 base pairs.

In certain embodiments, the 5′ and 3′ homology arms flank the break and are less than 100, 75, 50, 25, 15, 10 or 5 base pairs away from an edge of the break. In certain embodiments, the 5′ and 3′ homology arms flank an endogenous stop codon. In certain embodiments, the 5′ and 3′ homology arms flank a break located within about 500 base pairs (e.g., about 500 base pairs, about 450 base pairs, about 400 base pairs, about 350 base pairs, about 300 base pairs, about 250 base pairs, about 200 base pairs, about 150 base pairs, about 100 base pairs, about 50 base pairs, or about 25 base pairs) upstream (5′) of an endogenous stop codon, e.g., the stop codon of an essential gene. In certain embodiments, the 5′ homology arm encompasses an edge of the break.

Knock-In Cassette

In some embodiments, a knock-in cassette within the donor template comprises an exogenous coding sequence for the gene product of interest in frame with and downstream (3′) of an exogenous coding sequence or partial coding sequence of the essential gene. In some embodiments, a knock-in cassette within a donor template comprises an exogenous coding sequence for the gene product of interest in frame with and upstream (5′) of an exogenous coding sequence or partial coding sequence of an essential gene. In some embodiments, the knock-in cassette is a polycistronic knock-in cassette. In some embodiments, the knock-in cassette is a bicistronic knock-in cassette. In some embodiment the knock-in cassette does not comprise a reporter gene, e.g., a fluorescent reporter gene or an antibiotic resistance gene.

In some embodiments, a single essential gene locus will be targeted by two knock-in cassettes comprising different “cargo” sequences. In some embodiments, one allele will incorporate one knock-in cassette, while the other allele will incorporate the other knock-in cassette. In some embodiments, a gRNA utilized to generate an appropriate DNA break may be the same for each of the two different knock-in cassettes. In some embodiments, gRNAs utilized to generate appropriate DNA breaks for each of the two different knock-in cassettes may be different, such that the “cargo” sequence is incorporated at a different position for each allele. In some embodiments, such a different position for each allele may still be within the ultimate exons coding region. In some embodiments, such a different position for each allele may be within the penultimate exon (second to last), and/or ultimate (last) exons coding region. In some embodiments, such a different position for at least one of the alleles may be within the first exon. In some embodiments, such a different position for at least one of the alleles may be within the first or second exon.

In order to properly restore the essential gene coding region in the genetically modified cell (so that a functioning gene product is produced) the knock-in cassette does not need to comprise an exogenous coding sequence that corresponds to the entire coding sequence of the essential gene. Indeed, depending on the location of the break in the endogenous coding sequence of the essential gene it may be possible to restore the essential gene by providing a knock-in cassette that comprises a partial coding sequence of the essential gene, e.g., that corresponds to a portion of the endogenous coding sequence of the essential gene that spans the break and the entire region downstream of the break (minus the stop codon), and/or that corresponds to a portion of the endogenous coding sequence of the essential gene that spans the break and the entire region upstream of the break (up to and optionally including the start codon).

In order to minimize the size of the knock-in cassette it may in fact be advantageous, in some embodiments, to have the break located within the last 1500, 1000, 750, 500, 400, 300, 200, 100, or 50 base pairs of the endogenous coding sequence of the essential gene, i.e., towards the 3′ end of the coding sequence. In some embodiments, a base pair's location in a coding sequence may be defined 3′-to-5′ from an endogenous translational stop signal (e.g., a stop codon). In some embodiments, as used herein, an “endogenous coding sequence” can include both exonic and intronic base pairs, and refers to gene sequence occurring 5′ to an endogenous functional translational stop signal. In some embodiments, a break within an endogenous coding sequence comprises a break within one DNA strand. In some embodiments, a break within an endogenous coding sequence comprises a break within both DNA strands. In some embodiments, a break is located within the last 1000 base pairs of the endogenous coding sequence. In some embodiments, a break is located within the last 750 base pairs of the endogenous coding sequence. In some embodiments, a break is located within the last 600 base pairs of the endogenous coding sequence. In some embodiments, a break is located within the last 500 base pairs of the endogenous coding sequence. In some embodiments, a break is located within the last 400 base pairs of the endogenous coding sequence. In some embodiments, a break is located within the last 300 base pairs of the endogenous coding sequence. In some embodiments, a break is located within the last 250 base pairs of the endogenous coding sequence. In some embodiments, a break is located within the last 200 base pairs of the endogenous coding sequence. In some embodiments, a break is located within the last 150 base pairs of the endogenous coding sequence. In some embodiments, a break is located within the last 100 base pairs of the endogenous coding sequence. In some embodiments, a break is located within the last 75 base pairs of the endogenous coding sequence. In some embodiments, a break is located within the last 50 base pairs of the endogenous coding sequence. In some embodiments, a break is located within the last 21 base pairs of the endogenous coding sequence.

In some embodiments, the exogenous partial coding sequence of the essential gene in the knock-in cassette encodes a C-terminal fragment of a protein encoded by the essential gene, e.g., a fragment that is less than 500, 250, 150, 125, 100, 75, 50, 25, 20, 15 or 10 amino acids in length. In some embodiments, the exogenous partial coding sequence of the essential gene in the knock-in cassette is codon optimized. In some embodiments, the exogenous partial coding sequence of the essential gene in the knock-in cassette is codon optimized to eliminate at least one PAM site. In some embodiments, the exogenous partial coding sequence of the essential gene in the knock-in cassette is codon optimized to eliminate more than one PAM site. In some embodiments, the exogenous partial coding sequence of the essential gene in the knock-in cassette is codon optimized to eliminate all relevant nuclease specific PAM sites. In some embodiments, a C-terminal fragment of a protein encoded by the essential gene is about 140 amino acids in length. In some embodiments, a C-terminal fragment of a protein encoded by the essential gene is about 130 amino acids in length. In some embodiments, a C-terminal fragment of a protein encoded by the essential gene is about 120 amino acids in length. In some embodiments, the C-terminal fragment includes an amino acid sequence that is encoded by a region of the endogenous coding sequence of the essential gene that spans the break. In some embodiments, a C-terminal fragment includes an amino acid sequence that is encoded by a region of the endogenous coding sequence within 1 exon of the essential gene. In some embodiments, a C-terminal fragment includes an amino acid sequence that is encoded by a region of the endogenous coding sequence within 2 exons of the essential gene. In some embodiments, a C-terminal fragment includes an amino acid sequence that is encoded by a region of the endogenous coding sequence within 3 exons of the essential gene. In some embodiments, a C-terminal fragment includes an amino acid sequence that is encoded by a region of the endogenous coding sequence within 4 exons of the essential gene. In some embodiments, a C-terminal fragment includes an amino acid sequence that is encoded by a region of the endogenous coding sequence within 5 exons of the essential gene.

In some embodiments, the exogenous partial coding sequence of an essential gene in a knock-in cassette encodes a C-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette encodes a 20 amino acid C-terminal fragment of a protein encoded by an essential gene. In some embodiments, the exogenous partial coding sequence of an essential gene in a knock-in cassette encodes a 19 amino acid C-terminal fragment of a protein encoded by an essential gene. In some embodiments, the exogenous partial coding sequence of an essential gene in a knock-in cassette encodes a 18 amino acid C-terminal fragment of a protein encoded by an essential gene. In some embodiments, the exogenous partial coding sequence of an essential gene in a knock-in cassette encodes a 17 amino acid C-terminal fragment of a protein encoded by an essential gene. In some embodiments, the exogenous partial coding sequence of an essential gene in a knock-in cassette encodes a 16 amino acid C-terminal fragment of a protein encoded by an essential gene. In some embodiments, the exogenous partial coding sequence of an essential gene in a knock-in cassette encodes a 1 amino acid C-terminal fragment of a protein encoded by an essential gene.

In some embodiments, e.g., when the essential gene includes many exons as shown in the exemplary method of FIG. 3A, it may be advantageous to have the break within the last exon of the essential gene. In some embodiments, e.g., when the essential gene includes many exons as shown in the exemplary method of FIG. 3A, it may be advantageous to have the break within the penultimate exon of the essential gene. It is to be understood however that the present disclosure is not limited to any particular location for the break and that the available positions will vary depending on the nature and length of the essential gene and the length of the exogenous coding sequence for the gene product of interest. For example, for essential genes that include a few exons or when the gene product of interest is small it may be possible to locate the break in an upstream exon.

In order to minimize the size of the knock-in cassette it may in fact be advantageous, in some embodiments, to have the break located within the first 1500, 1000, 750, 500, 400, 300, 200, 100, or 50 base pairs of an endogenous coding sequence of the essential gene, i.e., starting from the 5′ end of a coding sequence. In some embodiments, a base pair's location in a coding sequence may be defined 5′-to-3′ from an endogenous translational start signal (e.g., a start codon). In some embodiments, as used herein, an “endogenous coding sequence” can include both exonic and intronic base pairs, and refers to gene sequence occurring 3′ to an endogenous functional translational start signal. In some embodiments, a break within an endogenous coding sequence comprises a break within one DNA strand. In some embodiments, a break within an endogenous coding sequence comprises a break within both DNA strands. In some embodiments, a break is located within the first 1000 base pairs of the endogenous coding sequence. In some embodiments, a break is located within the first 750 base pairs of a endogenous coding sequence. In some embodiments, a break is located within the first 600 base pairs of the endogenous coding sequence. In some embodiments, a break is located within the first 500 base pairs of the endogenous coding sequence. In some embodiments, a break is located within the first 400 base pairs of the endogenous coding sequence. In some embodiments, a break is located within the first 300 base pairs of the endogenous coding sequence. In some embodiments, a break is located within the first 250 base pairs of the endogenous coding sequence. In some embodiments, a break is located within the first 200 base pairs of the endogenous coding sequence. In some embodiments, a break is located within the first 150 base pairs of the endogenous coding sequence. In some embodiments, a break is located within the first 100 base pairs of the endogenous coding sequence. In some embodiments, a break is located within the first 75 base pairs of the endogenous coding sequence. In some embodiments, a break is located within the first 50 base pairs of the endogenous coding sequence. In some embodiments, a break is located within the first 21 base pairs of the endogenous coding sequence.

In some embodiments, the exogenous partial coding sequence of the essential gene in the knock-in cassette encodes an N-terminal fragment of a protein encoded by the essential gene, e.g., a fragment that is less than 500, 250, 150, 125, 100, 75, 50, 25, 20, 15 or 10 amino acids in length. In some embodiments, an N-terminal fragment of a protein encoded by the essential gene is about 140 amino acids in length. In some embodiments, an N-terminal fragment of a protein encoded by the essential gene is about 130 amino acids in length. In some embodiments, an N-terminal fragment of a protein encoded by the essential gene is about 120 amino acids in length. In some embodiments, an N-terminal fragment includes an amino acid sequence that is encoded by a region of the endogenous coding sequence of the essential gene that spans the break. In some embodiments, an N-terminal fragment includes an amino acid sequence that is encoded by a region of the endogenous coding sequence within 1 exon of the essential gene. In some embodiments, an N-terminal fragment includes an amino acid sequence that is encoded by a region of the endogenous coding sequence within 2 exons of the essential gene. In some embodiments, an N-terminal fragment includes an amino acid sequence that is encoded by a region of the endogenous coding sequence within 3 exons of the essential gene. In some embodiments, an N-terminal fragment includes an amino acid sequence that is encoded by a region of the endogenous coding sequence within 4 exons of the essential gene. In some embodiments, an N-terminal fragment includes an amino acid sequence that is encoded by a region of the endogenous coding sequence within 5 exons of the essential gene.

In some embodiments, the exogenous partial coding sequence of an essential gene in a knock-in cassette encodes an N-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette encodes a 20 amino acid N-terminal fragment of a protein encoded by an essential gene. In some embodiments, the exogenous partial coding sequence of an essential gene in a knock-in cassette encodes a 19 amino acid N-terminal fragment of a protein encoded by an essential gene. In some embodiments, the exogenous partial coding sequence of an essential gene in a knock-in cassette encodes a 18 amino acid N-terminal fragment of a protein encoded by an essential gene. In some embodiments, the exogenous partial coding sequence of an essential gene in a knock-in cassette encodes a 17 amino acid N-terminal fragment of a protein encoded by an essential gene. In some embodiments, the exogenous partial coding sequence of an essential gene in a knock-in cassette encodes a 16 amino acid N-terminal fragment of a protein encoded by an essential gene. In some embodiments, the exogenous partial coding sequence of an essential gene in a knock-in cassette encodes a 1 amino acid N-terminal fragment of a protein encoded by an essential gene.

In some embodiments, the exogenous coding sequence or partial coding sequence of the essential gene in the knock-in cassette is less than 100% identical to the corresponding endogenous coding sequence of the essential gene of the cell, e.g., less than 99%, 98%, 97%, 96%, 95%, 94%, 93%, 92%, 91%, 90%, 85%, 80%, 75%, 70%, 65%, 60%, 55% or less than 50% (i.e., when the two sequences are aligned using a standard pairwise sequence alignment tool that maximizes the alignment between the corresponding sequences). For example, in some embodiments, the exogenous coding sequence or partial coding sequence of the essential gene in the knock-in cassette is codon optimized relative to the corresponding endogenous coding sequence of the essential gene of the cell, e.g., to prevent further binding of a nuclease to the target site. Alternatively or additionally it may be codon optimized to reduce the likelihood of recombination after integration of the knock-in cassette into the genome of the cell and/or to increase expression of the gene product of the essential gene and/or the gene product of interest after integration of the knock-in cassette into the genome of the cell.

In some embodiments, a knock-in cassette comprises one or more nucleotides or base pairs that differ (e.g., are mutations) relative to an endogenous knock-in site. In some embodiments, such mutations in a knock-in cassette provide resistance to cutting by a nuclease. In some embodiments, such mutations in a knock-in cassette prevent a nuclease from cutting the target loci following homologous recombination. In some embodiments, such mutations in a knock-in cassette occur within one or more coding and/or non-coding regions of a target gene. In some embodiments, such mutations in a knock-in cassette are silent mutations. In some embodiments, such mutations in a knock-in cassette are silent and/or missense mutations.

In some embodiments, such mutations in a knock-in cassette occur within a target protospacer motif and/or a target protospacer adjacent motif (PAM) site. In some embodiments, a knock-in cassette includes a target protospacer motif and/or a PAM site that are saturated with silent mutations. In some embodiments, a knock-in cassette includes a target protospacer motif and/or a PAM site that are approximately 30%, 40%, 50%, 60%, 70%, 80%, or 90% saturated with silent mutations. In some embodiments, a knock-in cassette includes a target protospacer motif and/or a PAM site that are saturated with silent and/or missense mutations. In some embodiments, a knock-in cassette includes a target protospacer motif and/or a PAM site that comprise at least one mutation, at least 2 mutations, at least 3 mutations, at least 4 mutations, at least 5 mutations, at least 6 mutations, at least 7 mutations, at least 8 mutations, at least 9 mutations, at least 10 mutations, at least 11 mutations, at least 12 mutations, at least 13 mutations, at least 14 mutations, or at least 15 mutations.

In some embodiments, certain codons encoding certain amino acids in a target site cannot be mutated through codon-optimization without losing some portion of an endogenous proteins natural function. In some embodiments, certain codons encoding certain amino acids in a target site cannot be mutated through codon-optimization.

In some embodiments, the knock-in cassette is codon optimized in only a portion of the coding sequence. For example, in some embodiments, a knock-in cassette encodes a C-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette that has been codon optimized encodes a 20 amino acid C-terminal fragment of a protein encoded by an essential gene. In some embodiments, the exogenous partial coding sequence of an essential gene in a knock-in cassette that has been codon optimized encodes a 19 amino acid C-terminal fragment of a protein encoded by an essential gene. In some embodiments, the exogenous partial coding sequence of an essential gene in a knock-in cassette that has been codon optimized encodes a 18 amino acid C-terminal fragment of a protein encoded by an essential gene. In some embodiments, the exogenous partial coding sequence of an essential gene in a knock-in cassette that has been codon optimized encodes a 17 amino acid C-terminal fragment of a protein encoded by an essential gene. In some embodiments, the exogenous partial coding sequence of an essential gene in a knock-in cassette that has been codon optimized encodes a 16 amino acid C-terminal fragment of a protein encoded by an essential gene. In some embodiments, the exogenous partial coding sequence of an essential gene in a knock-in cassette that has been codon optimized encodes a 15 amino acid C-terminal fragment of a protein encoded by an essential gene. In some embodiments, the exogenous partial coding sequence of an essential gene in a knock-in cassette that has been codon optimized encodes a 14 amino acid C-terminal fragment of a protein encoded by an essential gene. In some embodiments, the exogenous partial coding sequence of an essential gene in a knock-in cassette that has been codon optimized encodes a 13 amino acid C-terminal fragment of a protein encoded by an essential gene. In some embodiments, the exogenous partial coding sequence of an essential gene in a knock-in cassette that has been codon optimized encodes a 12 amino acid C-terminal fragment of a protein encoded by an essential gene. In some embodiments, the exogenous partial coding sequence of an essential gene in a knock-in cassette that has been codon optimized encodes a 11 amino acid C-terminal fragment of a protein encoded by an essential gene. In some embodiments, the exogenous partial coding sequence of an essential gene in a knock-in cassette that has been codon optimized encodes a 10 amino acid C-terminal fragment of a protein encoded by an essential gene. In some embodiments, the exogenous partial coding sequence of an essential gene in a knock-in cassette that has been codon optimized encodes a 9 amino acid C-terminal fragment of a protein encoded by an essential gene. In some embodiments, the exogenous partial coding sequence of an essential gene in a knock-in cassette that has been codon optimized encodes a 8 amino acid C-terminal fragment of a protein encoded by an essential gene. In some embodiments, the exogenous partial coding sequence of an essential gene in a knock-in cassette that has been codon optimized encodes a 7 amino acid C-terminal fragment of a protein encoded by an essential gene. In some embodiments, the exogenous partial coding sequence of an essential gene in a knock-in cassette that has been codon optimized encodes a 6 amino acid C-terminal fragment of a protein encoded by an essential gene. In some embodiments, the exogenous partial coding sequence of an essential gene in a knock-in cassette that has been codon optimized encodes a 5 amino acid C-terminal fragment of a protein encoded by an essential gene. In some embodiments, the exogenous partial coding sequence of an essential gene in a knock-in cassette that has been codon optimized encodes an amino acid C-terminal fragment that is less than 5 amino acids of a protein encoded by an essential gene.

In some embodiments, the knock-in cassette is codon optimized in only a portion of the coding sequence. For example, in some embodiments, a knock-in cassette encodes an N-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette that has been codon optimized encodes a 20 amino acid N-terminal fragment of a protein encoded by an essential gene. In some embodiments, the exogenous partial coding sequence of an essential gene in a knock-in cassette that has been codon optimized encodes a 19 amino acid N-terminal fragment of a protein encoded by an essential gene. In some embodiments, the exogenous partial coding sequence of an essential gene in a knock-in cassette that has been codon optimized encodes a 18 amino acid N-terminal fragment of a protein encoded by an essential gene. In some embodiments, the exogenous partial coding sequence of an essential gene in a knock-in cassette that has been codon optimized encodes a 17 amino acid N-terminal fragment of a protein encoded by an essential gene. In some embodiments, the exogenous partial coding sequence of an essential gene in a knock-in cassette that has been codon optimized encodes a 16 amino acid N-terminal fragment of a protein encoded by an essential gene. In some embodiments, the exogenous partial coding sequence of an essential gene in a knock-in cassette that has been codon optimized encodes a 15 amino acid N-terminal fragment of a protein encoded by an essential gene. In some embodiments, the exogenous partial coding sequence of an essential gene in a knock-in cassette that has been codon optimized encodes a 14 amino acid N-terminal fragment of a protein encoded by an essential gene. In some embodiments, the exogenous partial coding sequence of an essential gene in a knock-in cassette that has been codon optimized encodes a 13 amino acid N-terminal fragment of a protein encoded by an essential gene. In some embodiments, the exogenous partial coding sequence of an essential gene in a knock-in cassette that has been codon optimized encodes a 12 amino acid N-terminal fragment of a protein encoded by an essential gene. In some embodiments, the exogenous partial coding sequence of an essential gene in a knock-in cassette that has been codon optimized encodes a 11 amino acid N-terminal fragment of a protein encoded by an essential gene. In some embodiments, the exogenous partial coding sequence of an essential gene in a knock-in cassette that has been codon optimized encodes a 10 amino acid N-terminal fragment of a protein encoded by an essential gene. In some embodiments, the exogenous partial coding sequence of an essential gene in a knock-in cassette that has been codon optimized encodes a 9 amino acid N-terminal fragment of a protein encoded by an essential gene. In some embodiments, the exogenous partial coding sequence of an essential gene in a knock-in cassette that has been codon optimized encodes a 8 amino acid N-terminal fragment of a protein encoded by an essential gene. In some embodiments, the exogenous partial coding sequence of an essential gene in a knock-in cassette that has been codon optimized encodes a 7 amino acid N-terminal fragment of a protein encoded by an essential gene. In some embodiments, the exogenous partial coding sequence of an essential gene in a knock-in cassette that has been codon optimized encodes a 6 amino acid N-terminal fragment of a protein encoded by an essential gene. In some embodiments, the exogenous partial coding sequence of an essential gene in a knock-in cassette that has been codon optimized encodes a 5 amino acid N-terminal fragment of a protein encoded by an essential gene. In some embodiments, the exogenous partial coding sequence of an essential gene in a knock-in cassette that has been codon optimized encodes an amino acid N-terminal fragment that is less than 5 amino acids of a protein encoded by an essential gene.

In some embodiments, the knock-in cassette comprises one or more sequences encoding a linker peptide, e.g., between an exogenous coding sequence or partial coding sequence of the essential gene and a “cargo” sequence and/or a regulatory element described herein. Such linker peptides are known in the art, any of which can be included in a knock-in cassette described herein. In some embodiments, the linker peptide comprises the amino acid sequence GSG.

In some embodiments, the knock-in cassette comprises other regulatory elements such as a polyadenylation sequence, and optionally a 3′ UTR sequence, downstream of the exogenous coding sequence for the gene product of interest. If a 3′UTR sequence is present, the 3′UTR sequence is positioned 3′ of the exogenous coding sequence and 5′ of the polyadenylation sequence.

In some embodiments, the knock-in cassette comprises other regulatory elements such as a 5′ UTR and a start codon, upstream of the exogenous coding sequence for the gene product of interest. If a 5′UTR sequence is present, the 5′UTR sequence is positioned 5′ of the “cargo” sequence and/or exogenous coding sequence.

Exemplary Homology Arms (HA)

In certain embodiments, a donor template comprises a 5′ and/or 3′ homology arm homologous to region of a GAPDH locus. In some embodiments, a donor template comprises a 5′ homology arm comprising or consisting of the sequence of SEQ ID NO:1, 2, or 3. In some embodiments, a 5′ homology arm comprises or consists of a sequence that is at least 85%, 90%, 95%, 98% or 99% identical to the sequence of SEQ ID NO: 1, 2, or 3. In some embodiments, a donor template comprises a 3′ homology arm comprising or consisting of the sequence of SEQ ID NO:4 or 5. In certain embodiments, a 3′ homology arm comprises or consists of a sequence that is at least 85%, 90%, 95%, 98% or 99% identical to the sequence of SEQ ID NO: 4 or 5.

In some embodiments, a donor template comprises a 5′ homology arm comprising SEQ ID NO: 1, and a 3′ homology arm comprising SEQ ID NO: 4. In some embodiments, a donor template comprises a 5′ homology arm comprising SEQ ID NO: 2, and a 3′ homology arm comprising SEQ ID NO: 4. In some embodiments, a donor template comprises a 5′ homology arm comprising SEQ ID NO: 3, and a 3′ homology arm comprising SEQ ID NO:5.

In some embodiments, a stretch of sequence flanking a nuclease cleavage site may be duplicated in both a 5′ and 3′ homology arm. In some embodiments, such a duplication is designed to optimize HDR efficiency. In some embodiments, one of the duplicated sequences may be codon optimized, while the other sequence is not codon optimized. In some embodiments, both of the duplicated sequences may be codon optimized. In some embodiments, codon optimization may remove a target PAM site. In some embodiments, a duplicated sequence may be no more than: 100 bp in length, 90 bp in length, 80 bp in length, 70 bp in length, 60 bp in length, 50 bp in length, 40 bp in length, 30 bp in length, or 20 bp in length.

-exemplary 5′ HA for knock-in cassette insertion at GAPDH locus
SEQ ID NO: 1
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAG
-exemplary 5′ HA for knock-in cassette insertion at GAPDH locus
SEQ ID NO: 2
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCT
-exemplary 5′ HA for knock-in cassette insertion at GAPDH locus
SEQ ID NO: 3
GGCTTTCCCATAATTTCCTTTCAAGGTGGGGAGGGAGGTAGAGGGGTGATGTGGGGAGTACGCT
GCAGGGCCTCACTCCTTTTGCAGACCACAGTCCATGCCATCACTGCCACCCAGAAGACTGTGGA
TGGCCCCTCCGGGAAACTGTGGCGTGATGGCCGCGGGGCTCTCCAGAACATCATCCCTGCCTCT
ACTGGCGCTGCCAAGGCTGTGGGCAAGGTCATCCCTGAGCTGAACGGGAAGCTCACTGGCATGG
CCTTCCGTGTCCCCACTGCCAACGTGTCAGTGGTGGACCTGACCTGCCGTCTAGAAAAACCTGC
CAAATATGATGACATCAAGAAGGTGGTGAAGCAGGCGTCGGAGGGCCCCCTCAAGGGCATCCTG
GGCTACACTGAGCACCAGGTGGTCTCCTCTGACTTCAACAGCGACACCCACTCCTCCACCTTTG
ACGCTGGGGCTGGCATTGCCCTCAACGACCACTTTGTCAAGCTCATCTCTTGGTACGACAATGA
GTTCGGATATAGCAATAGAGTGGTCGATCTGATGGCTCATATGGCTAGCAAAGAG
-exemplary 3′ HA for knock-in cassette insertion at GAPDH locus
SEQ ID NO: 4
ATTTGGCTACAGCAACAGGGTGGTGGACCTCATGGCCCACATGGCCTCCAAGGAGTAAGACCCC
TGGACCACCAGCCCCAGCAAGAGCACAAGAGGAAGAGAGAGACCCTCACTGCTGGGGAGTCCCT
GCCACACTCAGTCCCCCACCACACTGAATCTCCCCTCCTCACAGTTGCCATGTAGACCCCTTGA
AGAGGGGAGGGGCCTAGGGAGCCGCACCTTGTCATGTACCATCAATAAAGTACCCTGTGCTCAA
CCAGTTACTTGTCCTGTCTTATTCTAGGGTCTGGGGCAGAGGGGAGGGAAGCTGGGCTTGTGTC
AAGGTGAGACATTCTTGCTGGGGAGGGACCTGGTATGTTCTCCTCAGACTGAGGGTAGGGCCTC
CAAACAGCCTTGCTTGCTTCGAGAACCATTTGCTTCCCGCTCAGACGTCTTGAGTGCTACAGGA
AGCTGGCACCACTACTTCAGAGAACAAGGCCTTTTCCTCTCCTCGCTCCAGT
-exemplary 3′ HA for knock-in cassette insertion at GAPDH locus
SEQ ID NO: 5
AGACTGGCTCTTAAAAAGTGCAGGGTCTGGCGCCCTCTGGTGGCTGGCTCAGAAAAAGGGCCCT
GACAACTCTTTTCATCTTCTAGGTATGACAACGAATTTGGCTACAGCAACAGGGTGGTGGACCT
CATGGCCCACATGGCCTCCAAGGAGTAAGACCCCTGGACCACCAGCCCCAGCAAGAGCACAAGA
GGAAGAGAGAGACCCTCACTGCTGGGGAGTCCCTGCCACACTCAGTCCCCCACCACACTGAATC
TCCCCTCCTCACAGTTGCCATGTAGACCCCTTGAAGAGGGGAGGGGCCTAGGGAGCCGCACCTT
GTCATGTACCATCAATAAAGTACCCTGTGCTCAACCAGTTACTTGTCCTGTCTTATTCTAGGGT
CTGGGGCAGAGGGGAGGGAAGCTGGGCTTGTGTCAAGGTGAGACATTCTTGCTGGGGAGGGACC
TGGTATGTTCTCCTCAGACTGAGGGTAGGGCCTCCAAACAGCCTTGCTTGCT

In some embodiments, a donor template comprises a 5′ and/or 3′ homology arm homologous to a region of a TBP locus. In some embodiments, a donor template comprises a 5′ homology arm comprising or consisting of the sequence of SEQ ID NO:6, 7, or 8. In some embodiments, a 5′ homology arm comprises or consists of a sequence that is at least 85%, 90%, 95%, 98% or 99% identical to the sequence of SEQ ID NO: 6, 7, or 8. In some embodiments, a donor template comprises a 3′ homology arm comprising or consisting of the sequence of SEQ ID NO:9, 10, or 11. In certain embodiments, a 3′ homology arm comprises or consists of a sequence that is at least 85%, 90%, 95%, 98% or 99% identical to the sequence of SEQ ID NO: 9, 10, or 11.

In some embodiments, a donor template comprises a 5′ homology arm comprising SEQ ID NO: 6, and a 3′ homology arm comprising SEQ ID NO: 9. In some embodiments, a donor template comprises a 5′ homology arm comprising SEQ ID NO: 7, and a 3′ homology arm comprising SEQ ID NO: 10. In some embodiments, a donor template comprises a 5′ homology arm comprising SEQ ID NO: 8, and a 3′ homology arm comprising SEQ ID NO: 11.

-exemplary 5′ HA for knock-in cassette insertion at TBP locus
SEQ ID NO: 6
GCAGACTTCCATTTACAGTGAGGAGGTGAGCATTGCATTGAACAAAAGATGGCGTTTTCACTTG
GAATTAGTTATCTGAAGCTTTAGGATTCCTCAGCAATATGATTATGAGACAAGAAAGGAAGATT
CAGAAATGAGTCTAGTTGAAGGGAGCAATTCAGAGAAGAAGATTGAGTTGTTATCATTGCCGTC
CTGCTTGGTTTATGGCCTGGTTCAGGACCAAGGAGAGAAGTGTGAATACATGCCTCTTGAGCTA
TAGAATGAGACGCTGGAGTCACTAAGATGATTTTTTAAAAGTATTGTTTTATAAACAAAAATAA
GATTGTGACAAGGGATTCCACTATTAATGTTTTCATGCCTGTGCCTTAATCTGACTGGGTATGG
TGAGAATTGTGCTTGCAGCTTTAAGGTAAGAATTTTACCATCTTAATATGTTAAGAAGTGCCAT
TTCAGTCTCTCATCTCTACTCCAACTTGTCTTCTTAGGTGCTAAAGTCAGAGCCGAAATCTACG
AGGCCTTCGAGAACATCTACCCCATCCTGAAGGGCTTCAGAAAGACCACC
-exemplary 5′ HA for knock-in cassette insertion at TBP locus
SEQ ID NO: 7
CTGACCACAGCTCTGCAAGCAGACTTCCATTTACAGTGAGGAGGTGAGCATTGCATTGAACAAA
AGATGGCGTTTTCACTTGGAATTAGTTATCTGAAGCTTTAGGATTCCTCAGCAATATGATTATG
AGACAAGAAAGGAAGATTCAGAAATGAGTCTAGTTGAAGGCAGCAATTCAGAGAAGAAGATTCA
GTTGTTATCATTGCCGTCCTGCTTGGTTTATGGCCTGGTTCAGGACCAAGGAGAGAAGTGTGAA
TACATGCCTCTTGAGCTATAGAATGAGACGCTGGAGTCACTAAGATGATTTTTTAAAAGTATTG
TTTTATAAACAAAAATAAGATTGTGACAAGGGATTCCACTATTAATGTTTTCATGCCTGTGCCT
TAATCTGACTGGGTATGGTGAGAATTGTGCTTGCAGCTTTAAGGTAAGAATTTTACCATCTTAA
TATGTTAAGAAGTGCCATTTCAGTCTCTCATCTCTACTCCAACTTGTCTTCTTAGGGGCTAAAG
TGCGGGCCGAGATCTACGAGGCCTTCGAGAATATCTACCCCATCCTGAAGGGCTTCAGAAAGAC
CACC
-exemplary 5′ HA for knock-in cassette insertion at TBP locus
SEQ ID NO: 8
ACAAAAGATGGCGTTTTCACTTGGAATTAGTTATCTGAAGCTTTAGGATTCCTCAGCAATATGA
TTATGAGACAAGAAAGGAAGATTCAGAAATGAGTCTAGTTGAAGGCAGCAATTCAGAGAAGAAG
ATTCAGTTGTTATCATTGCCGTCCTGCTTGGTTTATGGCCTGGTTCAGGACCAAGGAGAGAAGT
GTGAATACATGCCTCTTGAGCTATAGAATGAGACGCTGGAGTCACTAAGATGATTTTTTAAAAG
TATTGTTTTATAAACAAAAATAAGATTGTGACAAGGGATTCCACTATTAATGTTTTCATGCCTG
TGCCTTAATCTGACTGGGTATGGTGAGAATTGTGCTTGCAGCTTTAAGGTAAGAATTTTACCAT
CTTAATATGTTAAGAAGTGCCATTTCAGTCTCTCATCTCTACTCCAACTTGTCTTCTTAGGTGC
TAAAGTCAGAGCAGAAATTTATGAAGCATTCGAGAACATCTACCCTATTCTAAAGGGATTCAGG
AAGACGACG
-exemplary 3′ HA for knock-in cassette insertion at TBP locus
SEQ ID NO: 9
CAGAAATTTATGAAGCATTTGAAAACATCTACCCTATTCTAAAGGGATTCAGGAAGACGACGTA
ATGGCTCTCATGTACCCTTGCCTCCCCCACCCCCTTCTTTTTTTTTTTTTAAACAAATCAGTTT
GTTTTGGTACCTTTAAATGGTGGTGTTGTGAGAAGATGGATGTTGAGTTGCAGGGTGTGGCACC
AGGTGATGCCCTTCTGTAAGTGCCCACCGCGGGATGCCGGGAAGGGGCATTATTTGTGCACTGA
GAACACCGCGCAGCGTGACTGTGAGTTGCTCATACCGTGCTGCTATCTGGGCAGCGCTGCCCAT
TTATTTATATGTAGATTTTAAACACTGCTGTTGACAAGTTGGTTTGAGGGAGAAAACTTTAAGT
GTTAAAGCCACCTCTATAATTGATTGGACTTTTTAATTTTAATGTTTTTCCCCATGAACCACAG
TTTTTATATTTCTACCAGAAAAGTAAAAATCTTTTTTAAAAGTGTTGTTTTT
-exemplary 3′ HA for knock-in cassette insertion at TBP locus
SEQ ID NO: 10
TAGGTGCTAAAGTCAGAGCAGAAATTTATGAAGCATTTGAAAACATCTACCCTATTCTAAAGGG
ATTCAGGAAGACGACGTAATGGCTCTCATGTACCCTTGCCTCCCCCACCCCCTTCTTTTTTTTT
TTTTAAACAAATCAGTTTGTTTTGGTACCTTTAAATGGTGGTGTTGTGAGAAGATGGATGTTGA
GTTGCAGGGTGTGGCACCAGGTGATGCCCTTCTGTAAGTGCCCACCGCGGGATGCCGGGAAGGG
GCATTATTTGTGCACTGAGAACACCGCGCAGCGTGACTGTGAGTTGCTCATACCGTGCTGCTAT
CTGGGCAGCGCTGCCCATTTATTTATATGTAGATTTTAAACACTGCTGTTGACAAGTTGGTTTG
AGGGAGAAAACTTTAAGTGTTAAAGCCACCTCTATAATTGATTGGAGTTTTTAATTTTAATGTT
TTTCCCCATGAACCACAGTTTTTATATTTCTACCAGAAAAGTAAAAATCTTT
-exemplary 3′ HA for knock-in cassette insertion at TBP locus
SEQ ID NO: 11
AAGGGATTCAGGAAGACGACGTAATGGCTCTCATGTACCCTTGCCTCCCCCACCCCCTTCTTTT
TTTTTTTTTAAACAAATCAGTTTGTTTTGGTACCTTTAAATGGTGGTGTTGTGAGAAGATGGAT
GTTGAGTTGCAGGGTGTGGCACCAGGTGATGCCCTTCTGTAAGTGCCCACCGCGGGATGCCGGG
AAGGGGCATTATTTGTGCACTGAGAACACCGCGCAGCGTGACTGTGAGTTGCTCATACCGTGCT
GCTATCTGGGCAGCGCTGCCCATTTATTTATATGTAGATTTTAAACACTGCTGTTGACAAGTTG
GTTTGAGGGAGAAAACTTTAAGTGTTAAAGCCACCTCTATAATTGATTGGACTTTTTAATTTTA
ATGTTTTTCCCCATGAACCACAGTTTTTATATTTCTACCAGAAAAGTAAAAATCTTTTTTAAAA
GTGTTGTTTTTCTAATTTATAACTCCTAGGGGTTATTTCTGTGCCAGACACA

In some embodiments, a donor template comprises a 5′ and/or 3′ homology arm homologous to a region of a G6PD locus. In some embodiments, a donor template comprises a 5′ homology arm comprising or consisting of the sequence of SEQ ID NO:12. In some embodiments, a 5′ homology arm comprises or consists of a sequence that is at least 85%, 90%, 95%, 98% or 99% identical to the sequence of SEQ ID NO: 12. In some embodiments, a donor template comprises a 3′ homology arm comprising or consisting of the sequence of SEQ ID NO:13. In certain embodiments, a 3′ homology arm comprises or consists of a sequence that is at least 85%, 90%, 95%, 98% or 99% identical to the sequence of SEQ ID NO:13.

In some embodiments, a donor template comprises a 5′ homology arm comprising SEQ ID NO: 12, and a 3′ homology arm comprising SEQ ID NO: 13.

-exemplary 5′ HA for knock-in cassette insertion at G6PD locus
SEQ ID NO: 12
GGCCCGGGGGACTCCACATGGTGGCAGGCAGTGGCATCAGCAAGACACTCTCTCCCTCACAGAA
CGTGAAGCTCCCTGACGCCTATGAGCGCCTCATCCTGGACGTCTTCTGCGGGAGCCAGATGCAC
TTCGTGCGCAGGTGAGGCCCAGCTGCCGGCCCCTGCATACCTGTGGGCTATGGGGTGGCCTTTG
CCCTCCCTCCCTGTGTGCCACCGGCCTCCCAAGCCATACCATGTCCCCTCAGCGACGAGCTCCG
TGAGGCCTGGCGTATTTTCACCCCACTGCTGCACCAGATTGAGCTGGAGAAGCCCAAGCCCATC
CCCTATATTTATGGCAGGTGAGGAAAGGGTGGGGGCTGGGGACAGAGCCCAGCGGGCAGGGGCG
GGGTGAGGGTGGAGCTACCTCATGCCTCTCCTCCACCCGTCACTCTCCAGCCGAGGCCCCACGG
AGGCAGACGAGCTGATGAAGAGAGTGGGCTTCCAGTACGAGGGAACCTACAAATGGGTCAACCC
TCACAAGGTG
-exemplary 3′ HA for knock-in cassette insertion at G6PD locus
SEQ ID NO: 13
GTGGGTGAACCCCCACAAGCTCTGAGCCCTGGGCACCCACCTCCACCCCCGCCACGGCCACCCT
CCTTCCCGCCGCCCGACCCCGAGTCGGGAGGACTCCGGGACCATTGACCTCAGCTGCACATTCC
TGGCCCCGGGCTCTGGCCACCCTGGCCCGCCCCTCGCTGCTGCTACTACCCGAGCCCAGCTACA
TTCCTCAGCTGCCAAGCACTCGAGACCATCCTGGCCCCTCCAGACCCTGCCTGAGCCCAGGAGC
TGAGTCACCTCCTCCACTCACTCCAGCCCAACAGAAGGAAGGAGGAGGGCGCCCATTCGTCTGT
CCCAGAGCTTATTGGCCACTGGGTCTCACTCCTGAGTGGGGCCAGGGTGGGAGGGAGGGACGAG
GGGGAGGAAAGGGGCGAGCACCCACGTGAGAGAATCTGCCTGTGGCCTTGCCCGCCAGCCTCAG
TGCCACTTGACATTCCTTGTCACCAGCAACATCTCGAGCCCCCTGGATGTCC

In some embodiments, a donor template comprises a 5′ and/or 3′ homology arm homologous to a region of a E2F4 locus. In some embodiments, a donor template comprises a 5′ homology arm comprising or consisting of the sequence of SEQ ID NO: 14, 15, or 16. In some embodiments, a 5′ homology arm comprises or consists of a sequence that is at least 85%, 90%, 95%, 98% or 99% identical to the sequence of SEQ ID NO: 14, 15, or 16. In some embodiments, a donor template comprises a 3′ homology arm comprising or consisting of the sequence of SEQ ID NO: 17, 18, or 19. In certain embodiments, a 3′ homology arm comprises or consists of a sequence that is at least 85%, 90%, 95%, 98% or 99% identical to the sequence of SEQ ID NO: 17, 18, or 19.

In some embodiments, a donor template comprises a 5′ homology arm comprising SEQ ID NO: 14, and a 3′ homology arm comprising SEQ ID NO: 17. In some embodiments, a donor template comprises a 5′ homology arm comprising SEQ ID NO: 15, and a 3′ homology arm comprising SEQ ID NO: 18. In some embodiments, a donor template comprises a 5′ homology arm comprising SEQ ID NO: 16, and a 3′ homology arm comprising SEQ ID NO: 19.

-exemplary 5′ HA for knock-in cassette insertion at E2F4 locus
SEQ ID NO: 14
CCAGGGGGCTGTAGTGGGGCCAGGCTGGACCTCTGTGCCCTGAGCATGGCTTTCTTGTTTTTCA
GTTTTGGAACTCCCCAAAGAGCTGTCAGAAATCTTTGATCCCACACGAGGTAGGCTGCTGCATT
CCTCCCTGAGGCTAGGGGTAAGGGACACAGCTCATTGGGTCCTATGGCTGTTTTCTTGCCCTTT
TGAGGACCTTGTTGTGGCGCTTATGGTAACTGGGGCAAAGGGTGAAGTTCCTGATGGGCAGGTG
GGGTTCCCTTTCCTGGGCTTTGGTGGGTGGAGAGGTGGGAGCTGGAATGTTAGTAACTGAGCTC
CCTCCATTCCCAGAGTGCATGAGCTCGGAGCTGCTGGAGGAGTTGATGTCCTCAGAAGGTGGGT
GGCCCTGGAAGGTGGGAGTGGGTGTGGGCAGGGGTTGGGCTGCTGCTAGGGGAGCCCTGGCCCA
GGGCCTGAGACTAGTGCTCTCTGCAGTGTTCGCCCCTCTGCTGAGACTTTCTCCTCCTCCTGGC
GACCACGACTACATCTACAACCTGGACGAGAGCGAGGGCGTGTGCGACCTGTTTGATGTGCCCG
TGCTGAACCTG
-exemplary 5′ HA for knock-in cassette insertion at E2F4 locus
SEQ ID NO: 15
CCAGGCTGGACCTCTGTGCCCTGAGCATGGCTTTCTTGTTTTTCAGTTTTGGAACTCCCCAAAG
AGCTGTCAGAAATCTTTGATCCCACACGAGGTAGGCTGCTGCATTCCTCCCTGAGGCTAGGGGT
AAGGGACACAGCTCATTGGGTCCTATGGCTGTTTTCTTGCCCTTTTGAGGACCTTGTTGTGGCG
CTTATGGTAACTGGGGCAAAGGGTGAAGTTCCTGATGGGCAGGTGGGGTTCCCTTTCCTGGGCT
TTGGTGGGTGGAGAGGTGGGAGCTGGAATGTTAGTAACTGAGCTCCCTCCATTCCCAGAGTGCA
TGAGCTCGGAGCTGCTGGAGGAGTTGATGTCCTCAGAAGGTGGGTGGCCCTGGAAGGTGGGAGT
GGGTGTGGGCAGGGGTTGGGCTGCTGCTAGGGGAGCCCTGGCCCAGGGCCTGAGACTAGTGCTC
TCTGCAGTGTTTGCCCCTCTGCTTCGTCTTAGTCCTCCTCCGGGCGACCACGACTACATCTACA
ACCTGGACGAGAGCGAGGGCGTGTGCGACCTGTTTGATGTGCCCGTGCTGAACCTG
-exemplary 5′ HA for knock-in cassette insertion at E2F4 locus
SEQ ID NO: 16
GTCAGAAATCTTTGATCCCACACGAGGTAGGCTGCTGCATTCCTCCCTGAGGCTAGGGGTAAGG
GACACAGCTCATTGGGTCCTATGGCTGTTTTCTTGCCCTTTTGAGGACCTTGTTGTGGCGCTTA
TGGTAACTGGGGCAAAGGGTGAAGTTCCTGATGGGCAGGTGGGGTTCCCTTTCCTGGGCTTTGG
TGGGTGGAGAGGTGGGAGCTGGAATGTTAGTAACTGAGCTCCCTCCATTCCCAGAGTGCATGAG
CTCGGAGCTGCTGGAGGAGTTGATGTCCTCAGAAGGTGGGTGGCCCTGGAAGGTGGGAGTGGGT
GTGGGCAGGGGTTGGGCTGCTGCTAGGGGAGCCCTGGCCCAGGGCCTGAGACTAGTGCTCTCTG
CAGTGTTTGCCCCTCTGCTTCGTCTTTCTCCACCCCCGGGAGACCACGATTATATCTACAACCT
GGACGAGAGTGAAGGTGTCTGTGACCTCTTCGACGTGCCCGTGCTCAACCTC
-exemplary 3′ HA for knock-in cassette insertion at E2F4 locus
SEQ ID NO: 17
CCACCCCCGGGAGACCACGATTATATCTACAACCTGGACGAGAGTGAAGGTGTCTGTGACCTCT
TTGATGTGCCTGTTCTCAACCTCTGACTGACAGGGACATGCCCTGTGTGGCTGGGACCCAGACT
GTCTGACCTGGGGGTTGCCTGGGGACCTCTCCCACCCGACCCCTACAGAGCTTGAGAGCCACAG
ACGCCTGGCTTCTCCGGCCTCCCCTCACCGCACAGTTCTGGCCACAGCTCCCGCTCCTGTGCTG
GCACTTCTGTGCTCGCAGAGCAGGGGAACAGGACTCAGCCCCCATCACCGTGGAGCCAAAGTGT
TTGCTTCTCCCTTTCTGCGGCCTTCGCCAGCCCAGGCTCGGCTGCCACCCAGTGGCACAGAACC
GAGGAGCTGCCATTACCCCCCATAGGGGGCAGTGTCTTGTTCCTGCCAGCCTCAGTGTCTTGCT
TCTGCCAGCTCCTTCCCCTAGGAGGGAAGGGTGGGGTGGAACTGGGCACATG
-exemplary 3′ HA for knock-in cassette insertion at E2F4 locus
SEQ ID NO: 18
ATTATATCTACAACCTGGACGAGAGTGAAGGTGTCTGTGACCTCTTTGATGTGCCTGTTCTCAA
CCTCTGACTGACAGGGACATGCCCTGTGTGGCTGGGACCCAGACTGTCTGACCTGGGGGTTGCC
TGGGGACCTCTCCCACCCGACCCCTACAGAGCTTGAGAGCCACAGACGCCTGGCTTCTCCGGCC
TCCCCTCACCGCACAGTTCTGGCCACAGCTCCCGCTCCTGTGCTGGCACTTCTGTGCTCGCAGA
GCAGGGGAACAGGACTCAGCCCCCATCACCGTGGAGCCAAAGTGTTTGCTTCTCCCTTTCTGCG
GCCTTCGCCAGCCCAGGCTCGGCTGCCACCCAGTGGCACAGAACCGAGGAGCTGCCATTACCCC
CCATAGGGGGCAGTGTCTTGTTCCTGCCAGCCTCAGTGTCTTGCTTCTGCCAGCTCCTTCCCCT
AGGAGGGAAGGGTGGGGTGGAACTGGGCACATGCCAGCACCACTTCTAGCTT
-exemplary 3′ HA for knock-in cassette insertion at E2F4 locus
SEQ ID NO: 19
TGACTGACAGGGACATGCCCTGTGTGGCTGGGACCCAGACTGTCTGACCTGGGGGTTGCCTGGG
GACCTCTCCCACCCGACCCCTACAGAGCTTGAGAGCCACAGACGCCTGGCTTCTCCGGCCTCCC
CTCACCGCACAGTTCTGGCCACAGCTCCCGCTCCTGTGCTGGCACTTCTGTGCTCGCAGAGCAG
GGGAACAGGACTCAGCCCCCATCACCGTGGAGCCAAAGTGTTTGCTTCTCCCTTTCTGCGGCCT
TCGCCAGCCCAGGCTCGGCTGCCACCCAGTGGCACAGAACCGAGGAGCTGCCATTACCCCCCAT
AGGGGGCAGTGTCTTGTTCCTGCCAGCCTCAGTGTCTTGCTTCTGCCAGCTCCTTCCCCTAGGA
GGGAAGGGTGGGGTGGAACTGGGCACATGCCAGCACCACTTCTAGCTTCCTTCGCTATCCCCCA
CCCCCTGACCCTCCAGCTCCTCCTGGCCCTCTCACGTGCCCACTTCTGCTGG

In some embodiments, a donor template comprises a 5′ and/or 3′ homology arm homologous to a region of a KIF11 locus. In some embodiments, a donor template comprises a 5′ homology arm comprising or consisting of the sequence of SEQ ID NO: 20, 21, or 22. In some embodiments, a 5′ homology arm comprises or consists of a sequence that is at least 85%, 90%, 95%, 98% or 99% identical to the sequence of SEQ ID NO: 20, 21, or 22. In some embodiments, a donor template comprises a 3′ homology arm comprising or consisting of the sequence of SEQ ID NO: 23, 24, or 25. In certain embodiments, a 3′ homology arm comprises or consists of a sequence that is at least 85%, 90%, 95%, 98% or 99% identical to the sequence of SEQ ID NO: 23, 24, or 25.

In some embodiments, a donor template comprises a 5′ homology arm comprising SEQ ID NO: 20, and a 3′ homology arm comprising SEQ ID NO: 23. In some embodiments, a donor template comprises a 5′ homology arm comprising SEQ ID NO: 21, and a 3′ homology arm comprising SEQ ID NO: 24. In some embodiments, a donor template comprises a 5′ homology arm comprising SEQ ID NO: 22, and a 3′ homology arm comprising SEQ ID NO: 25.

-exemplary 5′ HA for knock-in cassette insertion at KIF11 locus
SEQ ID NO: 20
AGAGCAGGGTTTCTTGACAGCAGTGCTATTGGCATTTTAAACTGGATAATTCTTTGTTGTGATG
GGCTTTCCTGTGGAGTGTACTATGTTGGTACACAAGAAAAACAGTGTACTATGTGAATACTCAC
TCAAAGCCAGTAGCACTCCCTGATTGTAACACCAAAAAAGTCTCTCAGCATTGCCAAATGTCCC
CTGTGGCAGCAGAATCACTCCCTGATGAGAACCACTACCCTGGAGTAAAATCTATAACTATGTC
TTAGAAAATAACACAGAAAATTAATATTTCTTTCACTCTACTCCTTCCATTAGTGATCAAATAA
AGAAGGCATTTGGCGCTACTTGCCAAATTGTTGGCTCAAACTTGTGCTGAACCTTTTTTGGTTT
TCTACACTTAAGTTTTTTTGCCTATAACCCAGAGAACTTTGAAAATAGAGTGTAGTTAATGTGT
ATCTAATGTTACTTTGTATTGACTTAATTTACCGGCCTTTAATCCACAGCATAAGAAGTCCCAC
GGCAAGGACAAAGAGAACCGGGGCATCAACACACTGGAACGGTCCAAGGTCGAGGAAACAACCG
AGCACCTGGTCACCAAGAGCAGACTGCCTCTGAGAGCCCAGATCAACCTG
-exemplary 5′ HA for knock-in cassette insertion at KIF11 locus
SEQ ID NO: 21
TTCCTGTGGACTGTACTATGTTGGTAGACAAGAAAAACAGTGTACTATGTGAATACTCACTCAA
AGCCAGTAGCACTCCCTGATTGTAACACCAAAAAAGTCTCTCAGCATTGCCAAATGTCCCCTGT
GGCAGCAGAATCACTCCCTGATGAGAACCACTACCCTGGAGTAAAATCTATAACTATGTCTTAG
AAAATAACACAGAAAATTAATATTTCTTTCACTCTACTCCTTCCATTAGTGATCAAATAAAGAA
GGCATTTGGCGCTACTTGCCAAATTGTTGGCTCAAACTTGTGCTGAACCTTTTTTGGTTTTCTA
CACTTAAGTTTTTTTGCCTATAACCCAGAGAACTTTGAAAATAGAGTGTAGTTAATGTGTATCT
AATGTTACTTTGTATTGAGTTAATTTTCCCGCCTTAAATCCACAGCATAAAAAATCACATGGAA
AAGACAAAGAAAACAGAGGCATTAACACACTGGAGAGGTCTAAAGTGGAAGAAACAACCGAGCA
CCTGGTCACCAAGAGCAGACTGCCTCTGAGAGCCCAGATCAACCTG
-exemplary 5′ HA for knock-in cassette insertion at KIF11 locus
SEQ ID NO: 22
TTAAACTGGATAATTCTTTGTTGTGATGGGCTTTCCTGTGGACTGTACTATGTTGGTACACAAG
AAAAACAGTGTACTATGTGAATACTCACTCAAAGCCAGTAGCACTCCCTGATTGTAACACCAAA
AAAGTCTCTCAGCATTGCCAAATGTCCCCTGTGGCAGCAGAATCACTCCCTGATGAGAACCACT
ACCCTGGAGTAAAATCTATAACTATGTCTTAGAAAATAACACAGAAAATTAATATTTCTTTCAC
TCTACTCCTTCCATTAGTGATCAAATAAAGAAGGCATTTGGCGCTACTTGCCAAATTGTTGGCT
CAAACTTGTGCTGAACCTTTTTTGGTTTTCTACACTTAAGTTTTTTTGCCTATAACCCAGAGAA
CTTTGAAAATAGAGTGTAGTTAATGTGTATCTAATGTTACTTTGTATTGACTTAATTTTCCCGC
CTTAAATCCACAGCATAAAAAATCACATGGAAAAGACAAAGAAAACAGAGGCATCAACACACTG
GAACGGTCCAAGGTCGAGGAAACAACCGAGCACCTGGTCACCAAGAGCAGACTGCCTCTGAGAG
CCCAGATCAACCTG
-exemplary 3′ HA for knock-in cassette insertion at KIF11 locus
SEQ ID NO: 23
AAAAAATCACATGGAAAAGACAAAGAAAACAGAGGCATTAACACACTGGAGAGGTCTAAAGTGG
AAGAAACTACAGAGCACTTGGTTACAAAGAGCAGATTACCTCTGCGAGCCCAGATCAACCTTTA
ATTCACTTGGGGGTTGGCAATTTTATTTTTAAAGAAAACTTAAAAATAAAACCTGAAACCCCAG
AACTTGAGCCTTGTGTATAGATTTTAAAAGAATATATATATCAGCCGGGCGCGGTGGCTCATGC
CTGTAATCCCAGCACTTTGGGAGGCTGAGGCGGGTGGATTGCTTGAGCCCAGGAGTTTGAGACC
AGCCTGGCCAACGTGGCAAAACCTCGTCTCTGTTAAAAATTAGCCGGGCGTGGTGGCACACTCC
TGTAATCCCAGCTACTGGGGAGGCTGAGGCACGAGAATCACTTGAACCCAGGAAGCGGGGTTGC
AGTGAGCCAAAGGTACACCACTACACTCCAGCCTGGGCAACAGAGCAAGACT
-exemplary 3′ HA for knock-in cassette insertion at KIF11 locus
SEQ ID NO: 24
AACTACAGAGCACTTGGCTACATAGAGCAGATTACCTCTGCGAGCCCAGATCAACCTTTAATTC
ACTTGGGGGTTGGCAATTTTATTTTTAAAGAAAACTTAAAAATAAAACCTGAAACCCCAGAACT
TGAGCCTTGTGTATAGATTTTAAAAGAATATATATATCAGCCGGGCGCGGTGGCTCATGCCTGT
AATCCCAGCACTTTGGGAGGCTGAGGCGGGTGGATTGCTTGAGCCCAGGAGTTTGAGACCAGCC
TGGCCAACGTGGCAAAACCTCGTCTCTGTTAAAAATTAGCCGGGCGTGGTGGCACACTCCTGTA
ATCCCAGCTACTGGGGAGGCTGAGGCACGAGAATCACTTGAACCCAGGAAGCGGGGTTGCAGTG
AGCCAAAGGTACACCACTACACTCCAGCCTGGGCAACAGAGCAAGACTCGGTCTCAAAAACAAA
ATTTAAAAAAGATATAAGGCAGTACTGTAAATTCAGTTGAATTTTGATATCT
-exemplary 3′ HA for knock-in cassette insertion at KIF11 locus
SEQ ID NO: 25
ATTAACACACTGGAGAGTTCTGAAGTGGAAGAAACTACAGAGCACTTGGTTACAAAGAGCAGAT
TACCTCTGCGAGCCCAGATCAACCTTTAATTCACTTGGGGGTTGGCAATTTTATTTTTAAAGAA
AACTTAAAAATAAAACCTGAAACCCCAGAACTTGAGCCTTGTGTATAGATTTTAAAAGAATATA
TATATCAGCCGGGCGCGGTGGCTCATGCCTGTAATCCCAGCACTTTGGGAGGCTGAGGCGGGTG
GATTGCTTGAGCCCAGGAGTTTGAGACCAGCCTGGCCAACGTGGCAAAACCTCGTCTCTGTTAA
AAATTAGCCGGGCGTGGTGGCACACTCCTGTAATCCCAGCTACTGGGGAGGCTGAGGCACGAGA
ATCACTTGAACCCAGGAAGCGGGGTTGCAGTGAGCCAAAGGTACACCACTACACTCCAGCCTGG
GCAACAGAGCAAGACTCGGTCTCAAAAACAAAATTTAAAAAAGATATAAGGC

Inverted Terminal Repeats (ITRs)

In certain embodiments, a donor template comprises an AAV derived sequence. In certain embodiments, a donor template comprises AAV derived sequences that are typical of an AAV construct, such as cis-acting 5′ and 3′ inverted terminal repeats (ITRs) (See, e.g., B. J. Carter, in “Handbook of Parvoviruses”, ed., P. Tijsser, CRC Press, pp. 155 168 (1990), which is incorporated in its entirety herein by reference). Generally, ITRs are able to form a hairpin. The ability to form a hairpin can contribute to an ITRs ability to self-prime, allowing primase-independent synthesis of a second DNA strand. ITRs also play a role in integration of AAV construct (e.g., a coding sequence) into a genome of a target cell. ITRs can also aid in efficient encapsidation of an AAV construct in an AAV particle.

In some embodiments, a donor template described herein is included within an rAAV particle (e.g., an AAV6 particle). In some embodiments, an ITR is or comprises about 145 nucleic acids. In some embodiments, all or substantially all of a sequence encoding an ITR is used. In some embodiments, an AAV ITR sequence may be obtained from any known AAV, including presently identified mammalian AAV types. In some embodiments an ITR is an AAV6 ITR.

An example of an AAV construct employed in the present disclosure is a “cis-acting” construct containing a cargo sequence (e.g., a donor template described herein), in which the donor template is flanked by 5′ or “left” and 3′ or “right” AAV ITR sequences. 5′ and left designations refer to a position of an ITR sequence relative to an entire construct, read left to right, in a sense direction. For example, in some embodiments, a 5′ or left ITR is an ITR that is closest to a target loci promoter (as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some embodiments, a 3′ or right ITR is an ITR that is closest to a polyadenylation sequence in a target loci (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3′/right ITR sequence). One of ordinary skill in the art would understand how to modify a given ITR sequence for use as either a 5′/left or 3′/right ITR, or an antisense version thereof.

For example, in some embodiments an ITR (e.g., a 5′ ITR) can have a sequence according to SEQ ID NO: 158. In some embodiments, an ITR (e.g., a 3′ ITR) can have a sequence according to SEQ ID NO: 159. In some embodiments, an ITR includes one or more modifications, e.g., truncations, deletions, substitutions or insertions, as is known in the art. In some embodiments, an ITR comprises fewer than 145 nucleotides, e.g., 127, 130, 134 or 141 nucleotides. For example, in some embodiments, an ITR comprises 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143 144, or 145 nucleotides.

A non-limiting example of 5′ AAV ITR sequences includes SEQ ID NO: 158. A non-limiting example of 3′ AAV ITR sequences includes SEQ ID NO: 159. In some embodiments, the 5′ and a 3′ AAV ITRs (e.g., SEQ ID NO: 158 and 159) flank a donor template described herein (e.g., a donor template comprising a 5′HA, a knock-in cassette, and a 3′ HA). The ability to modify ITR sequences is within the skill of the art. (See, e.g., texts such as Sambrook et al. “Molecular Cloning. A Laboratory Manual”, 2d ed., Cold Spring Harbor Laboratory, New York (1989); and K. Fisher et al., J Virol., 70:520 532 (1996), each of which is incorporated in its entirety herein by reference). In some embodiments, a 5′ ITR sequence is at least 85%, 90%, 95%, 98% or 99% identical to a 5′ ITR sequence represented by SEQ ID NO: 158. In some embodiments, a 3′ ITR sequence is at least 85%, 90%, 95%, 98% or 99% identical to a 3′ ITR sequence represented by SEQ ID NO: 159.

exemplary 5′ ITR for knock-in cassette insertion
SEQ ID NO: 158
CCTGCAGGCAGCTGCGCGCTCGCTCGCTCACTGAG
GCCGCCCGGGCAAAGCCCGGGCGTCGGGCGACCTT
TGGTCGCCCGGCCTCAGTGAGCGAGCGAGCGCGCA
GAGAGGGAGTGGCCAACTCCATCACTAGGGGTTCC
T
exemplary 3′ ITR for knock-in cassette insertion
SEQ ID NO: 159
AGGAACCCCTAGTGATGGAGTTGGCCACTCCCTCT
CTGCGCGCTCGCTCGCTCACTGAGGCCGGGCGACC
AAAGGTCGCCCGACGCCCGGGCTTTGCCCGGGCGG
CCTCAGTGAGCGAGCGAGCGCGCAGCTGCCTGCAG
G

Flanking Untranslated Regions, 5′ UTRs and 3′ UTRs

In some embodiments, a knock-in cassette described herein includes all or a portion of an untranslated region (UTR), such as a 5′ UTR and/or a 3′ UTR. UTRs of a gene are transcribed but not translated. A 5′ UTR starts at a transcription start site and continues to the start codon but does not include the start codon. A 3′ UTR starts immediately following the stop codon and continues until the transcriptional termination signal. The regulatory and/or control features of a UTR can be incorporated into any of the knock-in cassettes described herein to enhance or otherwise modulate the expression of an essential target gene loci and/or a cargo sequence.

Natural 5′ UTRs include a sequence that plays a role in translation initiation. In some embodiments, a 5′ UTR comprises sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding. Non-limiting examples of 5′ UTRs include those from the following genes: albumin, serum amyloid A, Apolipoprotein A/B/E, transferrin, alpha fetoprotein, erythropoietin, and Factor VIII.

In some embodiments, a UTR may comprise a non-endogenous regulatory region. In some embodiments, a UTR that comprises a non-endogenous regulatory region is a 3′ UTR. In some embodiments, a UTR that comprises a non-endogenous regulatory region is a 5′ UTR. In some embodiments, a non-endogenous regulatory region may be a target of at least one inhibitory nucleic acid. In some embodiments, an inhibitory nucleic acid inhibits expression and/or activity of a target gene. In some embodiments, an inhibitory nucleic acid is a short interfering RNA (siRNA), a short hairpin RNA (shRNA), a microRNA (miRNA), an antisense oligonucleotide, a guide RNA (gRNA), or a ribozyme. In some embodiments, an inhibitory nucleic acid is an endogenous molecule. In some embodiments, an inhibitory nucleic acid is a non-endogenous molecule. In some embodiments, an inhibitory nucleic acid displays a tissue specific expression pattern. In some embodiments, an inhibitory nucleic acid displays a cell specific expression pattern.

In some embodiments, a knock-in cassette may comprise more than one non-endogenous regulatory regions, e.g., two, three, four, five, six, seven, eight, nine, or ten regulatory regions. In some embodiments, a knock-in cassette may comprise four non-endogenous regulatory regions. In some embodiments, a construct may comprise more than one non-endogenous regulatory regions, wherein at least one of the more than one non-endogenous regulatory regions are not the same as at least one of the other non-endogenous regulatory regions.

In some embodiments, a 3′ UTR is found immediately 3′ to the stop codon of a gene of interest. In some embodiments, a 3′ UTR from an mRNA that is transcribed by a target cell can be included in any knock-in cassette described herein. In some embodiments, a 3′ UTR is derived from an endogenous target loci and may include all or part of the endogenous sequence. In some embodiments, a 3′ UTR sequence is at least 85%, 90%, 95% or 98% identical to the sequence of SEQ ID NO: 26.

exemplary 3′ UTR for knock-in cassette insertion
SEQ ID NO: 26
GCGGCCGCGTCGAGTCTAGAGGGCCCGTTTAAACC
CGCTGATCAGCCTCGA

Polyadenylation Sequences

In some embodiments, a knock-in cassette construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (Molecular Biology of the Cell, Third Edition by B. Alberts et al., Garland Publishing, 1994, which is incorporated herein by reference in its entirety). In some embodiments, a poly(A) signal sequence is positioned 3′ to a coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some embodiments, a poly(A) tail is added onto transcripts that contain a specific sequence, e.g., a polyadenylation (or poly(A)) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH) (Woychik et al., Proc. Natl. Acad Sci. US.A. 81(13):3944-3948, 1984; U.S. Pat. No. 5,122,458, each of which is incorporated herein by reference in its entirety), mouse-β-globin, mouse-α-globin (Orkin et al., EMBO J 4(2):453-456, 1985; Thein et al., Blood 71(2):313-319, 1988, each of which is incorporated herein by reference in its entirety), human collagen, polyoma virus (Batt et al., Mol. Cell Biol. 15(9):4783-4790, 1995, which is incorporated herein by reference in its entirety), the Herpes simplex virus thymidine kinase gene (HSV TK), IgG heavy-chain gene polyadenylation signal (US 2006/0040354, which is incorporated herein by reference in its entirety), human growth hormone (hGH) (Szymanski et al., Mol. Therapy 15(7):1340-1347, 2007, which is incorporated herein by reference in its entirety), the group comprising a SV40 poly(A) site, such as the SV40 late and early poly(A) site (Schek et al., Mol. Cell Biol. 12(12):5386-5393, 1992, which is incorporated herein by reference in its entirety).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

In some embodiments, a poly(A) signal sequence can be a synthetic polyadenylation site (see, e.g., the pCl-neo expression construct of Promega that is based on Levitt el al., Genes Dev. 3(7):1019-1025, 1989, which is incorporated herein by reference in its entirety). In some embodiments, a poly(A) signal sequence is the polyadenylation signal of soluble neuropilin-1 (sNRP) (AAATAAAATACGAAATG) (see, e.g., WO 05/073384, which is incorporated herein by reference in its entirety). In some embodiments, a poly(A) signal sequence comprises or consists of the SV40 poly(A) site. In some embodiments, a poly(A) signal sequence comprises or consists of SEQ ID NO: 27. In some embodiments, a poly(A) signal sequence comprises or consists of bGHpA. In some embodiments, a poly(A) signal sequence comprises or consists of SEQ ID NO: 28. Additional examples of poly(A) signal sequences are known in the art. In some embodiments, a poly(A) sequence is at least 85%, 90%, 95%, 98% or 99% identical to the sequence of SEQ ID NOs: 27 or 28.

exemplary SV40 poly(A) signal sequence
SEQ ID NO: 27
AACTTGTTTATTGCAGCTTATAATGGTTACAAATA
AAGCAATAGCATCACAAATTTCACAAATAAAGCAT
TTTTTTCACTGCATTCTAGTTGTGGTTTGTCCAAA
CTCATCAATGTATCTTA
exemplary bGH poly(A) signal sequence
SEQ ID NO: 28
CTGTGCCTTCTAGTTGCCAGCCATCTGTTGTTTGC
CCCTCCCCCGTGCCTTCCTTGACCCTGGAAGGTGC
CACTCCCACTGTCCTTTCCTAATAAAATGAGGAAA
TTGCATCGCATTGTCTGAGTAGGTGTCATTCTATT
CTGGGGGGTGGGGTGGGGCAGGACAGCAAGGGGGA
GGATTGGGAAGACAATAGCAGGCATGCTGGGGATG
CGGTGGGCTCTATGG

IRES and 2A Elements

in some embodiments, the knock-in cassette comprises a regulatory element that enables expression of the gene product encoded by the essential gene and the gene product of interest as separate gene products, e.g., an IRES or 2A element located between the exogenous coding sequence or partial coding sequence of the essential gene and the exogenous coding sequence for the gene product of interest.

In some embodiments, a knock-in cassette may comprise multiple gene products of interest (e.g., at least two gene products of interest). In some embodiments, gene products of interest may be separated by a regulatory element that enables expression of the at least two gene products of interest as more than one gene product, e.g., an IRES or 2A element located between the at least two coding sequences, facilitating creation of at least two peptide products.

Internal Ribosome Entry Site (IRES) elements are one type of regulatory element that are commonly used for this purpose. As is well known in the art, IRES elements allow for initiation of translation from an internal region of the mRNA and hence expression of two separate proteins from the same mRNA transcript. IRES was originally discovered in poliovirus RNA, where it promotes translation of the viral genome in eukaryotic cells. Since then, a variety of IRES sequences have been discovered—many from viruses, but also some from cellular mRNAs, e.g., see Mokrejs et al., Nucleic Acids Res. 2006; 34 (Database issue):D125-D130.

2A elements are another type of regulatory element that are commonly used for this purpose. These 2A elements encode so-called “self-cleaving” 2A peptides which are short peptides (about 20 amino acids) that were first discovered in picornaviruses. The term “self-cleaving” is not entirely accurate, as these peptides are thought to function by making the ribosome skip the synthesis of a peptide bond at the C-terminus of a 2A element, leading to separation between the end of the 2A sequence and the next peptide downstream. The “cleavage” occurs between the Glycine (G) and Proline (P) residues found on the C-terminus meaning the upstream cistron, i.e., protein encoded by the essential gene will have a few additional residues from the 2A peptide added to the end, while the downstream cistron, i.e., gene product of interest will start with the Proline (P).

Table 2 below lists the four commonly used 2A peptides (an optional GSG sequence is sometimes added to the N-terminal end of the peptide to improve cleavage efficiency). There are many potential 2A peptides that may be suitable for methods and compositions described herein (see e.g., Luke et al., Occurrence, function and evolutionary origins of ‘2A-like’ sequences in virus genomes. J Gen Virol. 2008). Those skilled in the art know that the choice of specific 2A peptide for a particular knock-in cassette will ultimately depend on a number of factors such as cell type or experimental conditions. Those skilled in the art will recognize that nucleotide sequences encoding specific 2A peptides can vary while still encoding a peptide suitable for inducing a desired cleavage event.

TABLE 2
Exemplary 2A peptide sequences

| SEQ | 2A | |
|---|---|---|
| ID | pep- | |
| NO: | tide | Sequence |
| 29 | T2A | EGRGSLLTCGDVEENPGP |
| 30 | P2A | ATNFSLLKQAGDVEENPGP |
| 31 | E2A | QCTNYALLKLAGDVESNPGP |
| 32 | F2A | VKQTLNFDLLKLAGDVESNPGP |
| 33 | T2A | GAGGGCAGAGGAAGTCTTCTAACATGCGGTGAGGTGGAGG |
| | | AGAATCCTGGCCCG |
| 34 | P2A | GGAAGCGGAGCTACTAACTTCAGCCTGCTGAAGCAGGCTG |
| | | GAGACGTGGAGGAGAACCCTGGACCT |
| 35 | E2A | CAGTGTACTAATTATGCTCTCTTGAAATTGGCTGGAGATG |
| | | TTGAGAGCAACCCTGGACCT |
| 36 | F2A | GTGAAACAGACTTTGAATTTTGACCTTCTCAAGTTGGCGG |
| | | GAGACGTGGAGTCCAACCCTGGACCT |
| 37 | IRES | CCCCTCTCCCTCCCCCCCCCCTAACGTTACTGGCCGAAGC |
| | | CGCTTGGAATAAGGCCGGTGTGCGTTTGTCTATATGTTAT |
| | | TTTCCACCATATTGCCGTCTTTTGGCAATGTGAGGGCCCG |
| | | GAAACCTGGCCCTGTCTTCTTGACGAGCATTCCTAGGGGT |
| | | CTTTCCCCTCTCGCCAAAGGAATGCAAGGTCTGTTGAATG |
| | | TCGTGAAGGAAGCAGTTCCTCTGGAAGCTTCTTGAAGACA |
| | | AACAACGTCTGTAGCGACCCTTTGCAGGCAGCGGAACCCC |
| | | CCACCTGGCGACAGGTGCCTCTGCGGCCAAAAGCCACGTG |
| | | TATAAGATACACCTGCAAAGGCGGCACAACCCCAGTGCCA |
| | | CGTTGTGAGTTGGATAGTTGTGGAAAGAGTCAAATGGCTC |
| | | TCCTCAAGCGTATTCAACAAGGGGCTGAAGGATGCCCAGA |
| | | AGGTACCCCATTGTATGGGATCTGATCTGGGGCCTCGGTG |
| | | CACATGCTTTAGATGTGTTTAGTCGAGGTTAAAAAAACGT |
| | | CTAGGCCCCCCGAACCACGGGGACGTGGTTTTCCTTTGAA |
| | | AAACACGATGATAA |

Essential Genes

An essential gene can be any gene that is essential for the survival and/or the proliferation of the cell. In some embodiments, an essential gene is a housekeeping gene that is essential for survival of all cell types, e.g., a gene listed in Table 3. See also other housekeeping genes discussed in Eisenberg, Trends in Gen, 2014; 30(3):119-20 and Moein et al., Adv, Biomed Res. 2017; 6:15, Additional genes that are essential for various cell types, including iPSCs/ESCs, are listed in Table 4 (see also the essential genes discussed in Yilmaz et al., Nat. Cell Biol. 2018; 20:610-619 the entire contents of which are incorporated herein by reference).

In some embodiments the essential gene is GAPDH and the DNA nuclease causes a break in exon 9, e.g., a double-strand break. In some embodiments the essential gene is TBP and the DNA nuclease causes a break in exon 7, or exon 8, e.g., a double-strand break. In some embodiments the essential gene is E2F4 and the DNA nuclease causes a break in exon 10, e.g., a double-strand break. In some embodiments the essential gene is G6PD and the DNA nuclease causes a break in exon 13, e.g., a double-strand break. In some embodiments the essential gene is KIF11 and the DNA nuclease causes a break in exon 22, e.g., a double-strand break.

TABLE 3
Exemplary housekeeping genes

| Ensembl ID | Gene Symbol | Ensembl ID | Gene Symbol |
|---|---|---|---|
| ENSG00000075624 | ACTB | ENSG00000231500 | RPS18 |
| ENSG00000116459 | ATP5F1 | ENSG00000112592 | TBP |
| ENSG00000166710 | B2M | ENSG00000072274 | TFRC |
| ENSG00000111640 | GAPDH | ENSG00000164924 | YWHAZ |
| ENSG00000169919 | GUSB | ENSG00000089157 | RPLP0 |
| ENSG00000165704 | HPRT1 | ENSG00000142541 | RPL13A |
| ENSG00000102144 | PGK1 | ENSG00000147604 | RPL7 |
| ENSG00000196262 | PPIA | ENSG00000205250 | E2F4 |
| ENSG00000138160 | KIF11 | ENSG00000160211 | G6PD |

TABLE 4
Additional exemplary essential genes

| Ensembl ID | Gene Symbol | Ensembl ID | Gene Symbol |
|---|---|---|---|
| ENSG00000111704 | NANOG | ENSG00000181449 | SOX2 |
| ENSG00000179059 | ZFP42 | ENSG00000136997 | MYC |
| ENSG00000136826 | KLF4 | ENSG00000175166 | PSMD2 |
| ENSG00000118655 | DCLRE1B | ENSG00000070614 | NDST1 |
| ENSG00000172409 | CLP1 | ENSG00000115484 | CCT4 |
| ENSG00000082898 | XPO1 | ENSG00000100890 | KIAA0391 |
| ENSG00000114867 | EIF4G1 | ENSG00000149474 | CSRP2BP |
| ENSG00000115866 | DARS | ENSG00000102738 | MRPS31 |
| ENSG00000204628 | GNB2L1 | ENSG00000136104 | RNASEH2B |
| ENSG00000198242 | RPL23A | ENSG00000106246 | PTCD1 |
| ENSG00000158526 | TSR2 | ENSG00000248919 | ATP5J2-PTCD1 |
| ENSG00000125450 | NUP85 | ENSG00000138663 | COPS4 |
| ENSG00000134371 | CDC73 | ENSG00000115368 | WDR75 |
| ENSG00000164941 | INTS8 | ENSG00000128564 | VGF |
| ENSG00000055483 | USP36 | ENSG00000128191 | DGCR8 |
| ENSG00000258366 | RTEL1 | ENSG00000008294 | SPAG9 |
| ENSG00000188846 | RPL14 | ENSG00000131475 | VPS25 |
| ENSG00000247626 | MARS2 | ENSG00000105523 | FAM83E |
| ENSG00000095787 | WAC | ENSG00000172269 | DPAGT1 |
| ENSG00000108094 | CUL2 | ENSG00000170312 | CDK1 |
| ENSG00000185946 | RNPC3 | ENSG00000104131 | EIF3J |
| ENSG00000154473 | BUB3 | ENSG00000150753 | CCT5 |
| ENSG00000204394 | VARS | ENSG00000140443 | IGF1R |
| ENSG00000103051 | COG4 | ENSG00000010292 | NCAPD2 |
| ENSG00000104738 | MCM4 | ENSG00000171763 | SPATA5L1 |
| ENSG00000117222 | RBBP5 | ENSG00000180098 | TRNAU1AP |
| ENSG00000082516 | GEMIN5 | ENSG00000168374 | ARF4 |
| ENSG00000100162 | CENPM | ENSG00000173812 | EIF1 |
| ENSG00000141456 | PELP1 | ENSG00000100554 | ATP6V1D |
| ENSG00000137807 | KIF23 | ENSG00000072756 | TRNT1 |
| ENSG00000112685 | EXOC2 | ENSG00000135372 | NAT10 |
| ENSG00000125995 | ROMO1 | ENSG00000178394 | HTR1A |
| ENSG00000136891 | TEX10 | ENSG00000128272 | ATF4 |
| ENSG00000173113 | TRMT112 | ENSG00000204070 | SYS1 |
| ENSG00000075914 | EXOSC7 | ENSG00000137815 | RTF1 |
| ENSG00000119523 | ALG2 | ENSG00000198026 | ZNF335 |
| ENSG00000244038 | DDOST | ENSG00000117410 | ATP6V0B |
| ENSG00000108175 | ZMIZ1 | ENSG00000112739 | PRPF4B |
| ENSG00000129691 | ASH2L | ENSG00000129347 | KRI1 |
| ENSG00000183207 | RUVBL2 | ENSG00000221818 | EBF2 |
| ENSG00000055044 | NOP58 | ENSG00000198431 | TXNRD1 |
| ENSG00000204315 | FKBPL | ENSG00000104979 | C19orf53 |
| ENSG00000187522 | HSPA14 | ENSG00000136709 | WDR33 |
| ENSG00000169375 | SIN3A | ENSG00000149100 | EIF3M |
| ENSG00000143748 | NVL | ENSG00000125835 | SNRPB |
| ENSG00000021776 | AQR | ENSG00000116698 | SMG7 |
| ENSG00000132467 | UTP3 | ENSG00000087586 | AURKA |
| ENSG00000087470 | DNM1L | ENSG00000169230 | PRELID1 |
| ENSG00000130811 | EIF3G | ENSG00000143799 | PARP1 |
| ENSG00000180198 | RCC1 | ENSG00000146731 | CCT6A |
| ENSG00000101407 | TTI1 | ENSG00000163877 | SNIP1 |
| ENSG00000116455 | WDR77 | ENSG00000215421 | ZNF407 |
| ENSG00000135763 | URB2 | ENSG00000197724 | PHF2 |
| ENSG00000133316 | WDR74 | ENSG00000172590 | MRPL52 |
| ENSG00000189091 | SF3B3 | ENSG00000175203 | DCTN2 |
| ENSG00000109917 | ZNF259 | ENSG00000149273 | RPS3 |
| ENSG00000130640 | TUBGCP2 | ENSG00000204822 | MRPL53 |
| ENSG00000011376 | LARS2 | ENSG00000109775 | UFSP2 |
| ENSG00000135249 | RINT1 | ENSG00000165733 | BMS1 |
| ENSG00000126883 | NUP214 | ENSG00000104671 | DCTN6 |
| ENSG00000163510 | CWC22 | ENSG00000175224 | ATG13 |
| ENSG00000101138 | CSTF1 | ENSG00000142541 | RPL13A |
| ENSG00000104221 | BRF2 | ENSG00000173805 | HAP1 |
| ENSG00000125630 | POLR1B | ENSG00000115750 | TAF1B |
| ENSG00000083896 | YTHDC1 | ENSG00000165688 | PMPCA |
| ENSG00000105726 | ATP13A1 | ENSG00000159720 | ATP6V0D1 |
| ENSG00000105618 | PRPF31 | ENSG00000074201 | CLNS1A |
| ENSG00000117748 | RPA2 | ENSG00000158417 | EIF5B |
| ENSG00000143294 | PRCC | ENSG00000196588 | MKL1 |
| ENSG00000156239 | N6AMT1 | ENSG00000138614 | VWA9 |
| ENSG00000143384 | MCL1 | ENSG00000124571 | XPO5 |
| ENSG00000113407 | TARS | ENSG00000198000 | NOL8 |
| ENSG00000086589 | RBM22 | ENSG00000181991 | MRPS11 |
| ENSG00000133119 | RFC3 | ENSG00000149823 | VPS51 |
| ENSG00000052749 | RRP12 | ENSG00000151348 | EXT2 |
| ENSG00000103047 | TANGO6 | ENSG00000162396 | PARS2 |
| ENSG00000142751 | GPN2 | ENSG00000204843 | DCTN1 |
| ENSG00000101057 | MYBL2 | ENSG00000177302 | TOP3A |
| ENSG00000176915 | ANKLE2 | ENSG00000142684 | ZNF593 |
| ENSG00000071127 | WDR1 | ENSG00000074800 | ENO1 |
| ENSG00000106344 | RBM28 | ENSG00000167513 | CDT1 |
| ENSG00000100316 | RPL3 | ENSG00000141101 | NOB1 |
| ENSG00000139131 | YARS2 | ENSG00000047315 | POLR2B |
| ENSG00000182831 | C16orf72 | ENSG00000131966 | ACTR10 |
| ENSG00000167325 | RRM1 | ENSG00000115875 | SRSF7 |
| ENSG00000172262 | ZNF131 | ENSG00000186141 | POLR3C |
| ENSG00000007168 | PAFAH1B1 | ENSG00000108424 | KPNB1 |
| ENSG00000117174 | ZNHIT6 | ENSG00000111845 | PAK1IP1 |
| ENSG00000196497 | IPO4 | ENSG00000148832 | PAOX |
| ENSG00000188566 | NDOR1 | ENSG00000156017 | C9orf41 |
| ENSG00000183091 | NEB | ENSG00000198901 | PRC1 |
| ENSG00000011304 | PTBP1 | ENSG00000134001 | EIF2S1 |
| ENSG00000109805 | NCAPG | ENSG00000146918 | NCAPG2 |
| ENSG00000123154 | WDR83 | ENSG00000144713 | RPL32 |
| ENSG00000147416 | ATP6V1B2 | ENSG00000185122 | HSF1 |
| ENSG00000163961 | RNF168 | ENSG00000167658 | EEF2 |
| ENSG00000163811 | WDR43 | ENSG00000164190 | NIPBL |
| ENSG00000143624 | INTS3 | ENSG00000163902 | RPN1 |
| ENSG00000101161 | PRPF6 | ENSG00000244045 | TMEM199 |
| ENSG00000130726 | TRIM28 | ENSG00000143476 | DTL |
| ENSG00000165494 | PCF11 | ENSG00000149503 | INCENP |
| ENSG00000053900 | ANAPC4 | ENSG00000071243 | ING3 |
| ENSG00000168255 | POLR2J3 | ENSG00000186073 | C15orf41 |
| ENSG00000129534 | MIS18BP1 | ENSG00000088836 | SLC4A11 |
| ENSG00000164754 | RAD21 | ENSG00000136273 | HUS1 |
| ENSG00000120158 | RCL1 | ENSG00000005007 | UPF1 |
| ENSG00000161016 | RPL8 | ENSG00000070010 | UFD1L |
| ENSG00000030066 | NUP160 | ENSG00000106263 | EIF3B |
| ENSG00000099624 | ATP5D | ENSG00000213024 | NUP62 |
| ENSG00000116120 | FARSB | ENSG00000067191 | CACNB1 |
| ENSG00000115233 | PSMD14 | ENSG00000179091 | CYC1 |
| ENSG00000086504 | MRPL28 | ENSG00000113312 | TTC1 |
| ENSG00000160752 | FDPS | ENSG00000085831 | TTC39A |
| ENSG00000049541 | RFC2 | ENSG00000118197 | DDX59 |
| ENSG00000148688 | RPP30 | ENSG00000134871 | COL4A2 |
| ENSG00000114573 | ATP6V1A | ENSG00000088986 | DYNLL1 |
| ENSG00000086200 | IPO11 | ENSG00000138778 | CENPE |
| ENSG00000119720 | NRDE2 | ENSG00000106244 | PDAP1 |
| ENSG00000058262 | SEC61A1 | ENSG00000177600 | RPLP2 |
| ENSG00000073111 | MCM2 | ENSG00000112081 | SRSF3 |
| ENSG00000138160 | KIF11 | ENSG00000100413 | POLR3H |
| ENSG00000215193 | PEX26 | ENSG00000172508 | CARNS1 |
| ENSG00000161057 | PSMC2 | ENSG00000147123 | NDUFB11 |
| ENSG00000187514 | PTMA | ENSG00000119953 | SMNDC1 |
| ENSG00000135829 | DHX9 | ENSG00000111640 | GAPDH |
| ENSG00000058729 | RIOK2 | ENSG00000117899 | MESDC2 |
| ENSG00000110330 | BIRC2 | ENSG00000075624 | ACTB |
| ENSG00000141759 | TXNL4A | ENSG00000163166 | IWS1 |
| ENSG00000166986 | MARS | ENSG00000114503 | NCBP2 |
| ENSG00000153774 | CFDP1 | ENSG00000198522 | GPN1 |
| ENSG00000130177 | CDC16 | ENSG00000099899 | TRMT2A |
| ENSG00000241553 | ARPC4 | ENSG00000181544 | FANCB |
| ENSG00000132604 | TERF2 | ENSG00000136982 | DSCC1 |
| ENSG00000114982 | KANSL3 | ENSG00000068366 | ACSL4 |
| ENSG00000213780 | GTF2H4 | ENSG00000062716 | VMP1 |
| ENSG00000139343 | SNRPF | ENSG00000111802 | TDP2 |
| ENSG00000101189 | MRGBP | ENSG00000185627 | PSMD13 |
| ENSG00000079246 | XRCC5 | ENSG00000020426 | MNAT1 |
| ENSG00000196943 | NOP9 | ENSG00000113734 | BNIP1 |
| ENSG00000122965 | RBM19 | ENSG00000102241 | HTATSF1 |
| ENSG00000132383 | RPA1 | ENSG00000160789 | LMNA |
| ENSG00000094880 | CDC23 | ENSG00000062822 | POLD1 |
| ENSG00000213639 | PPP1CB | ENSG00000168944 | CEP120 |
| ENSG00000109911 | ELP4 | ENSG00000139718 | SETD1B |
| ENSG00000180957 | PITPNB | ENSG00000132792 | CTNNBL1 |
| ENSG00000122257 | RBBP6 | ENSG00000173540 | GMPPB |
| ENSG00000173145 | NOC3L | ENSG00000128789 | PSMG2 |
| ENSG00000179115 | FARSA | ENSG00000196365 | LONP1 |
| ENSG00000105171 | POP4 | ENSG00000160214 | RRP1 |
| ENSG00000148303 | RPL7A | ENSG00000179041 | RRS1 |
| ENSG00000167508 | MVD | ENSG00000143106 | PSMA5 |
| ENSG00000115541 | HSPE1 | ENSG00000168411 | RFWD3 |
| ENSG00000170445 | HARS | ENSG00000073584 | SMARCE1 |
| ENSG00000168496 | FEN1 | ENSG00000175334 | BANF1 |
| ENSG00000141367 | CLTC | ENSG00000077152 | UBE2T |
| ENSG00000087191 | PSMC5 | ENSG00000173611 | SCAI |
| ENSG00000163159 | VPS72 | ENSG00000171720 | HDAC3 |
| ENSG00000130741 | EIF2S3 | ENSG00000182197 | EXT1 |
| ENSG00000168495 | POLR3D | ENSG00000114346 | ECT2 |
| ENSG00000071894 | CPSF1 | ENSG00000124214 | STAU1 |
| ENSG00000058600 | POLR3E | ENSG00000126254 | RBM42 |
| ENSG00000100726 | TELO2 | ENSG00000127184 | COX7C |
| ENSG00000165501 | LRR1 | ENSG00000174276 | ZNHIT2 |
| ENSG00000113575 | PPP2CA | ENSG00000177971 | IMP3 |
| ENSG00000116922 | C1orf109 | ENSG00000104872 | PIH1D1 |
| ENSG00000073712 | FERMT2 | ENSG00000132155 | RAF1 |
| ENSG00000174437 | ATP2A2 | ENSG00000163872 | YEATS2 |
| ENSG00000176407 | KCMF1 | ENSG00000119906 | FAM178A |
| ENSG00000140525 | FANCI | ENSG00000217930 | PAM16 |
| ENSG00000101182 | PSMA7 | ENSG00000197498 | RPF2 |
| ENSG00000130204 | TOMM40 | ENSG00000130348 | QRSL1 |
| ENSG00000239306 | RBM14 | ENSG00000147536 | GINS4 |
| ENSG00000248643 | RBM14-RBM4 | ENSG00000174748 | RPL15 |
| ENSG00000172113 | NME6 | ENSG00000159147 | DONSON |
| ENSG00000136448 | NMT1 | ENSG00000157593 | SLC35B2 |
| ENSG00000186166 | CCDC84 | ENSG00000181938 | GINS3 |
| ENSG00000166233 | ARIH1 | ENSG00000187446 | CHP1 |
| ENSG00000111877 | MCM9 | ENSG00000070371 | CLTCL1 |
| ENSG00000204316 | MRPL38 | ENSG00000096063 | SRPK1 |
| ENSG00000101868 | POLA1 | ENSG00000141564 | RPTOR |
| ENSG00000107951 | MTPAP | ENSG00000108474 | PIGL |
| ENSG00000039650 | PNKP | ENSG00000187741 | FANCA |
| ENSG00000123064 | DDX54 | ENSG00000213465 | ARL2 |
| ENSG00000183955 | SETD8 | ENSG00000117593 | DARS2 |
| ENSG00000138107 | ACTR1A | ENSG00000171863 | RPS7 |
| ENSG00000244005 | NFS1 | ENSG00000117395 | EBNA1BP2 |
| ENSG00000188986 | NELFB | ENSG00000111142 | METAP2 |
| ENSG00000018699 | TTC27 | ENSG00000113272 | THG1L |
| ENSG00000167112 | TRUB2 | ENSG00000117360 | PRPF3 |
| ENSG00000100393 | EP300 | ENSG00000221978 | CCNL2 |
| ENSG00000101639 | CEP192 | ENSG00000163832 | ELP6 |
| ENSG00000126461 | SCAF1 | ENSG00000108852 | MPP2 |
| ENSG00000172171 | TEFM | ENSG00000175832 | ETV4 |
| ENSG00000135913 | USP37 | ENSG00000185359 | HGS |
| ENSG00000135624 | CCT7 | ENSG00000120705 | ETF1 |
| ENSG00000100804 | PSMB5 | ENSG00000108384 | RAD51C |
| ENSG00000175792 | RUVBL1 | ENSG00000036257 | CUL3 |
| ENSG00000183431 | SF3A3 | ENSG00000152382 | TADA1 |
| ENSG00000108773 | KAT2A | ENSG00000114742 | WDR48 |
| ENSG00000100949 | RABGGTA | ENSG00000214026 | MRPL23 |
| ENSG00000151503 | NCAPD3 | ENSG00000105671 | DDX49 |
| ENSG00000111880 | RNGTT | ENSG00000104731 | KLHDC4 |
| ENSG00000168883 | USP39 | ENSG00000010256 | UQCRC1 |
| ENSG00000151461 | UPF2 | ENSG00000154743 | TSEN2 |
| ENSG00000105486 | LIG1 | ENSG00000178896 | EXOSC4 |
| ENSG00000111300 | NAA25 | ENSG00000168393 | DTYMK |
| ENSG00000144559 | TAMM41 | ENSG00000035928 | RFC1 |
| ENSG00000137574 | TGS1 | ENSG00000048707 | VPS13D |
| ENSG00000172273 | HINFP | ENSG00000154832 | CXXC1 |
| ENSG00000133112 | TPT1 | ENSG00000130985 | UBA1 |
| ENSG00000167986 | DDB1 | ENSG00000065150 | IPO5 |
| ENSG00000125319 | C17orf53 | ENSG00000161800 | RACGAP1 |
| ENSG00000113161 | HMGCR | ENSG00000142534 | RPS11 |
| ENSG00000100941 | PNN | ENSG00000136003 | ISCU |
| ENSG00000139697 | SBNO1 | ENSG00000065000 | AP3D1 |
| ENSG00000135336 | ORC3 | ENSG00000100401 | RANGAP1 |
| ENSG00000101115 | SALL4 | ENSG00000196230 | TUBB |
| ENSG00000100902 | PSMA6 | ENSG00000181555 | SETD2 |
| ENSG00000141141 | DDX52 | ENSG00000055950 | MRPL43 |
| ENSG00000254093 | PINX1 | ENSG00000188389 | PDCD1 |
| ENSG00000184445 | KNTC1 | ENSG00000165684 | SNAPC4 |
| ENSG00000089053 | ANAPC5 | ENSG00000147533 | GOLGA7 |
| ENSG00000111602 | TIMELESS | ENSG00000064313 | TAF2 |
| ENSG00000145592 | RPL37 | ENSG00000137154 | RPS6 |
| ENSG00000106615 | RHEB | ENSG00000104886 | PLEKHJ1 |
| ENSG00000180817 | PPA1 | ENSG00000122882 | ECD |
| ENSG00000110172 | CHORDC1 | ENSG00000184967 | NOC4L |
| ENSG00000137876 | RSL24D1 | ENSG00000088325 | TPX2 |
| ENSG00000104408 | EIF3E | ENSG00000183520 | UTP11L |
| ENSG00000143436 | MRPL9 | ENSG00000179051 | RCC2 |
| ENSG00000108883 | EFTUD2 | ENSG00000157510 | AFAP1L1 |
| ENSG00000140740 | UQCRC2 | ENSG00000066379 | ZNRD1 |
| ENSG00000211456 | SACM1L | ENSG00000172115 | CYCS |
| ENSG00000131051 | RBM39 | ENSG00000086827 | ZW10 |
| ENSG00000136758 | YME1L1 | ENSG00000109534 | GAR1 |
| ENSG00000112578 | BYSL | ENSG00000175387 | SMAD2 |
| ENSG00000163781 | TOPBP1 | ENSG00000115947 | ORC4 |
| ENSG00000106628 | POLD2 | ENSG00000010072 | SPRTN |
| ENSG00000132952 | USPL1 | ENSG00000185163 | DDX51 |
| ENSG00000168538 | TRAPPC11 | ENSG00000177370 | TIMM22 |
| ENSG00000168488 | ATXN2L | ENSG00000076924 | XAB2 |
| ENSG00000022277 | RTFDC1 | ENSG00000124562 | SNRPC |
| ENSG00000179988 | PSTK | ENSG00000127586 | CHTF18 |
| ENSG00000092199 | HNRNPC | ENSG00000066117 | SMARCD1 |
| ENSG00000156831 | NSMCE2 | ENSG00000177494 | ZBED2 |
| ENSG00000125691 | RPL23 | ENSG00000133401 | PDZD2 |
| ENSG00000083520 | DIS3 | ENSG00000127554 | GFER |
| ENSG00000115761 | NOL10 | ENSG00000117697 | NSL1 |
| ENSG00000173894 | CBX2 | ENSG00000184659 | FOXD4L4 |
| ENSG00000243147 | MRPL33 | ENSG00000204828 | FOXD4L2 |
| ENSG00000139618 | BRCA2 | ENSG00000110200 | ANAPC15 |
| ENSG00000109519 | GRPEL1 | ENSG00000169291 | SHE |
| ENSG00000203760 | CENPW | ENSG00000132313 | MRPL35 |
| ENSG00000166851 | PLK1 | ENSG00000115816 | CEBPZ |
| ENSG00000121579 | NAA50 | ENSG00000243667 | WDR92 |
| ENSG00000163608 | C3orf17 | ENSG00000107959 | PITRM1 |
| ENSG00000005075 | POLR2J | ENSG00000103035 | PSMD7 |
| ENSG00000148606 | POLR3A | ENSG00000163946 | FAM208A |
| ENSG00000160949 | TONSL | ENSG00000178057 | NDUFAF3 |
| ENSG00000128159 | TUBGCP6 | ENSG00000170540 | ARL6IP1 |
| ENSG00000125449 | ARMC7 | ENSG00000091009 | RBM27 |
| ENSG00000122406 | RPL5 | ENSG00000205609 | EIF3CL |
| ENSG00000126226 | PCID2 | ENSG00000165526 | RPUSD4 |
| ENSG00000159377 | PSMB4 | ENSG00000120314 | WDR55 |
| ENSG00000167967 | E4F1 | ENSG00000013275 | PSMC4 |
| ENSG00000141076 | CIRH1A | ENSG00000131931 | THAP1 |
| ENSG00000069248 | NUP133 | ENSG00000155660 | PDIA4 |
| ENSG00000242372 | EIF6 | ENSG00000162607 | USP1 |
| ENSG00000087269 | NOP14 | ENSG00000109606 | DHX15 |
| ENSG00000163468 | CCT3 | ENSG00000261949 | LOC100507003 |
| ENSG00000140326 | CDAN1 | ENSG00000130589 | HELZ2 |
| ENSG00000146834 | MEPCE | ENSG00000145734 | BDP1 |
| ENSG00000143222 | UFC1 | ENSG00000103194 | USP10 |
| ENSG00000110871 | COQ5 | ENSG00000076201 | PTPN23 |
| ENSG00000119285 | HEATR1 | ENSG00000140854 | KATNB1 |
| ENSG00000145386 | CCNA2 | ENSG00000164053 | ATRIP |
| ENSG00000164109 | MAD2L1 | ENSG00000167088 | SNRPD1 |
| ENSG00000185347 | C14orf80 | ENSG00000154781 | CCDC174 |
| ENSG00000134748 | PRPF38A | ENSG00000115446 | UNC50 |
| ENSG00000070061 | IKBKAP | ENSG00000177700 | POLR2L |
| ENSG00000099995 | SF3A1 | ENSG00000162063 | CCNF |
| ENSG00000100029 | PES1 | ENSG00000152904 | GGPS1 |
| ENSG00000130255 | RPL36 | ENSG00000151657 | KIN |
| ENSG00000085231 | AK6 | ENSG00000182810 | DDX28 |
| ENSG00000187145 | MRPS21 | ENSG00000006744 | ELAC2 |
| ENSG00000062650 | WAPAL | ENSG00000116898 | MRPS15 |
| ENSG00000122484 | RPAP2 | ENSG00000255072 | PIGY |
| ENSG00000090861 | AARS | ENSG00000130332 | LSM7 |
| ENSG00000161888 | SPC24 | ENSG00000051180 | RAD51 |
| ENSG00000087087 | SRRT | ENSG00000178171 | AMER3 |
| ENSG00000134910 | STT3A | ENSG00000254901 | MEF2BNB |
| ENSG00000161526 | SAP30BP | ENSG00000149925 | ALDOA |
| ENSG00000068654 | POLR1A | ENSG00000100604 | CHGA |
| ENSG00000140983 | RHOT2 | ENSG00000172602 | RND1 |
| ENSG00000184708 | EIF4ENIF1 | ENSG00000138592 | USP8 |
| ENSG00000100479 | POLE2 | ENSG00000172613 | RAD9A |
| ENSG00000134440 | NARS | ENSG00000132196 | HSD17B7 |
| ENSG00000014164 | ZC3H3 | ENSG00000151849 | CENPJ |
| ENSG00000113812 | ACTR8 | ENSG00000105221 | AKT2 |
| ENSG00000145331 | TRMT10A | ENSG00000185504 | C17orf70 |
| ENSG00000110104 | CCDC86 | ENSG00000025796 | SEC63 |
| ENSG00000164163 | ABCE1 | ENSG00000168438 | CDC40 |
| ENSG00000167863 | ATP5H | ENSG00000163918 | RFC4 |
| ENSG00000176946 | THAP4 | ENSG00000152147 | GEMIN6 |
| ENSG00000169251 | NMD3 | ENSG00000166887 | VPS39 |
| ENSG00000166226 | CCT2 | ENSG00000018625 | ATP1A2 |
| ENSG00000131747 | TOP2A | ENSG00000163346 | PBXIP1 |
| ENSG00000267673 | FDX1L | ENSG00000135966 | TGFBRAP1 |
| ENSG00000108559 | NUP88 | ENSG00000099901 | RANBP1 |
| ENSG00000104957 | CCDC130 | ENSG00000010327 | STAB1 |
| ENSG00000167522 | ANKRD11 | ENSG00000163344 | PMVK |
| ENSG00000130706 | ADRM1 | ENSG00000102921 | N4BP1 |
| ENSG00000048162 | NOP16 | ENSG00000177150 | FAM210A |
| ENSG00000159210 | SNF8 | ENSG00000158042 | MRPL17 |
| ENSG00000113360 | DROSHA | ENSG00000124659 | TBCC |
| ENSG00000108296 | CWC25 | ENSG00000113593 | PPWD1 |
| ENSG00000161395 | PGAP3 | ENSG00000188306 | LRRIQ4 |
| ENSG00000089195 | TRMT6 | ENSG00000074966 | TXK |
| ENSG00000185838 | GNB1L | ENSG00000228049 | POLR2J2 |
| ENSG00000101146 | RAE1 | ENSG00000133226 | SRRM1 |
| ENSG00000092853 | CLSPN | ENSG00000121577 | POPDC2 |
| ENSG00000107949 | BCCIP | ENSG00000130876 | SLC7A10 |
| ENSG00000159079 | C21orf59 | ENSG00000130810 | PPAN |
| ENSG00000137947 | GTF2B | ENSG00000243207 | PPAN-P2RY11 |
| ENSG00000160948 | VPS28 | ENSG00000081248 | CACNA1S |
| ENSG00000065427 | KARS | ENSG00000153201 | RANBP2 |
| ENSG00000102978 | POLR2C | ENSG00000126698 | DNAJC8 |
| ENSG00000182154 | MRPL41 | ENSG00000103018 | CYB5B |
| ENSG00000139168 | ZCRB1 | ENSG00000130816 | DNMT1 |
| ENSG00000175110 | MRPS22 | ENSG00000102103 | PQBP1 |
| ENSG00000177084 | POLE | ENSG00000120253 | NUP43 |
| ENSG00000197681 | TBC1D3 | ENSG00000164327 | RICTOR |
| ENSG00000053501 | USE1 | ENSG00000139719 | VPS33A |
| ENSG00000121879 | PIK3CA | ENSG00000168566 | SNRNP48 |
| ENSG00000108278 | ZNHIT3 | ENSG00000063244 | U2AF2 |
| ENSG00000161547 | SRSF2 | ENSG00000108423 | TUBD1 |
| ENSG00000129083 | COPB1 | ENSG00000164880 | INTS1 |
| ENSG00000012048 | BRCA1 | ENSG00000148297 | MED22 |
| ENSG00000171314 | PGAM1 | ENSG00000185825 | BCAP31 |
| ENSG00000112159 | MDN1 | ENSG00000084623 | EIF3I |
| ENSG00000174243 | DDX23 | ENSG00000066422 | ZBTB11 |
| ENSG00000096401 | CDC5L | ENSG00000119041 | GTF3C3 |
| ENSG00000128513 | POT1 | ENSG00000083093 | PALB2 |
| ENSG00000071859 | FAM50A | ENSG00000120699 | EXOSC8 |
| ENSG00000100084 | HIRA | ENSG00000166135 | HIF1AN |
| ENSG00000100813 | ACIN1 | ENSG00000188976 | NOC2L |
| ENSG00000005100 | DHX33 | ENSG00000102974 | CTCF |
| ENSG00000101158 | NELFCD | ENSG00000148229 | POLE3 |
| ENSG00000115946 | PNO1 | ENSG00000167118 | URM1 |
| ENSG00000188647 | PTAR1 | ENSG00000176386 | CDC26 |
| ENSG00000146007 | ZMAT2 | ENSG00000110063 | DCPS |
| ENSG00000241837 | ATP5O | ENSG00000089737 | DDX24 |
| ENSG00000113643 | RARS | ENSG00000119383 | PPP2R4 |
| ENSG00000162521 | RBBP4 | ENSG00000143319 | ISG20L2 |
| ENSG00000116830 | TTF2 | ENSG00000141552 | ANAPC11 |
| ENSG00000187555 | USP7 | ENSG00000155506 | LARP1 |
| ENSG00000137216 | TMEM63B | ENSG00000144867 | SRPRB |
| ENSG00000161904 | LEMD2 | ENSG00000093000 | NUP50 |
| ENSG00000241945 | PWP2 | ENSG00000107937 | GTPBP4 |
| ENSG00000134982 | APC | ENSG00000083635 | NUFIP1 |
| ENSG00000156983 | BRPF1 | ENSG00000174527 | MYO1H |
| ENSG00000164346 | NSA2 | ENSG00000124641 | MED20 |
| ENSG00000223496 | EXOSC6 | ENSG00000240694 | PNMA2 |
| ENSG00000113569 | NUP155 | ENSG00000122012 | SV2C |
| ENSG00000080986 | NDC80 | ENSG00000017260 | ATP2C1 |
| ENSG00000143374 | TARS2 | ENSG00000179965 | ZNF771 |
| ENSG00000104835 | SARS2 | ENSG00000126216 | TUBGCP3 |
| ENSG00000152253 | SPC25 | ENSG00000126814 | TRMT5 |
| ENSG00000088356 | PDRG1 | ENSG00000101945 | SUV39H1 |
| ENSG00000044574 | HSPA5 | ENSG00000182185 | RAD51B |
| ENSG00000116874 | WARS2 | ENSG00000163681 | SLMAP |
| ENSG00000204531 | POU5F1 | ENSG00000179295 | PTPN11 |
| ENSG00000004779 | NDUFAB1 | ENSG00000004487 | KDM1A |
| ENSG00000161981 | SNRNP25 | ENSG00000136100 | VPS36 |
| ENSG00000126457 | PRMT1 | ENSG00000168066 | SF1 |
| ENSG00000142507 | PSMB6 | ENSG00000197181 | PIWIL2 |
| ENSG00000164808 | SPIDR | ENSG00000128908 | INO80 |
| ENSG00000234972 | TBC1D3C | ENSG00000102144 | PGK1 |
| ENSG00000144554 | FANCD2 | ENSG00000007923 | DNAJC11 |
| ENSG00000147383 | NSDHL | ENSG00000143514 | TP53BP2 |
| ENSG00000165732 | DDX21 | ENSG00000076650 | GPATCH1 |
| ENSG00000155975 | VPS37A | ENSG00000130749 | ZC3H4 |
| ENSG00000002822 | MADIL1 | ENSG00000062582 | MRPS24 |
| ENSG00000179271 | GADD45GIP1 | ENSG00000087085 | ACHE |
| ENSG00000101452 | DHX35 | ENSG00000197976 | AKAP17A |
| ENSG00000074071 | MRPS34 | ENSG00000100028 | SNRPD3 |
| ENSG00000169045 | HNRNPH1 | ENSG00000128731 | HERC2 |
| ENSG00000087510 | TFAP2C | ENSG00000134014 | ELP3 |
| ENSG00000105819 | PMPCB | ENSG00000181163 | NPM1 |
| ENSG00000204351 | SKIV2L | ENSG00000148444 | COMMD3 |
| ENSG00000160783 | PMF1 | ENSG00000095319 | NUP188 |
| ENSG00000152234 | ATP5A1 | ENSG00000169564 | PCBP1 |
| ENSG00000127463 | EMC1 | ENSG00000182208 | MOB2 |
| ENSG00000124228 | DDX27 | ENSG00000055070 | SZRD1 |
| ENSG00000100319 | ZMAT5 | ENSG00000182473 | EXOC7 |
| ENSG00000065183 | WDR3 | ENSG00000136930 | PSMB7 |
| ENSG00000058272 | PPP1R12A | ENSG00000107863 | ARHGAP21 |
| ENSG00000136628 | EPRS | ENSG00000197223 | C1D |
| ENSG00000163017 | ACTG2 | ENSG00000184270 | HIST2H2AB |
| ENSG00000104884 | ERCC2 | ENSG00000161036 | LRWD1 |
| ENSG00000166483 | WEE1 | ENSG00000144736 | SHQ1 |
| ENSG00000135837 | CEP350 | ENSG00000137100 | DCTN3 |
| ENSG00000104897 | SF3A2 | ENSG00000131149 | GSE1 |
| ENSG00000140598 | EFTUD1 | ENSG00000214753 | HNRNPUL2 |
| ENSG00000143774 | GUK1 | ENSG00000111358 | GTF2H3 |
| ENSG00000085721 | RRN3 | ENSG00000147677 | EIF3H |
| ENSG00000172053 | QARS | ENSG00000125676 | THOC2 |
| ENSG00000165934 | CPSF2 | ENSG00000149554 | CHEK1 |
| ENSG00000052802 | MSMO1 | ENSG00000176476 | CCDC101 |
| ENSG00000135476 | ESPL1 | ENSG00000147596 | PRDM14 |
| ENSG00000174177 | CTU2 | ENSG00000092094 | OSGEP |
| ENSG00000120438 | TCP1 | ENSG00000155393 | HEATR3 |
| ENSG00000170892 | TSEN34 | ENSG00000083845 | RPS5 |
| ENSG00000204574 | ABCF1 | ENSG00000148296 | SURF6 |
| ENSG00000175376 | EIF1AD | ENSG00000162613 | FUBP1 |
| ENSG00000146263 | MMS22L | ENSG00000182220 | ATP6AP2 |
| ENSG00000121022 | COPS5 | ENSG00000115163 | CENPA |
| ENSG00000168090 | COPS6 | ENSG00000176225 | RTTN |
| ENSG00000167491 | GATAD2A | ENSG00000176208 | ATAD5 |
| ENSG00000084072 | PPIE | ENSG00000254827 | SLC22A18AS |
| ENSG00000115268 | RPS15 | ENSG00000128708 | HAT1 |
| ENSG00000163938 | GNL3 | ENSG00000106400 | ZNHIT1 |
| ENSG00000151665 | PIGF | ENSG00000123219 | CENPK |
| ENSG00000148843 | PDCD11 | ENSG00000264424 | MYH4 |
| ENSG00000141736 | ERBB2 | ENSG00000066468 | FGFR2 |
| ENSG00000103168 | TAF1C | ENSG00000095059 | DHPS |
| ENSG00000105401 | CDC37 | ENSG00000110921 | MVK |
| ENSG00000163933 | RFT1 | ENSG00000141556 | TBCD |
| ENSG00000122085 | MTERFD2 | ENSG00000196305 | IARS |
| ENSG00000164032 | H2AFZ | ENSG00000131055 | COX4I2 |
| ENSG00000140943 | MBTPS1 | ENSG00000153789 | FAM92B |
| ENSG00000198952 | SMG5 | ENSG00000088930 | XRN2 |
| ENSG00000169021 | UQCRFS1 | ENSG00000145220 | LYAR |
| ENSG00000013810 | TACC3 | ENSG00000172809 | RPL38 |
| ENSG00000105258 | POLR2I | ENSG00000108788 | MLX |
| ENSG00000167978 | SRRM2 | ENSG00000197170 | PSMD12 |
| ENSG00000095564 | BTAF1 | ENSG00000225899 | FRG2B |
| ENSG00000138095 | LRPPRC | ENSG00000174886 | NDUFA11 |
| ENSG00000063978 | RNF4 | ENSG00000172058 | SERF1A |
| ENSG00000162368 | CMPK1 | ENSG00000205572 | SERF1B |
| ENSG00000140829 | DHX38 | ENSG00000242485 | MRPL20 |
| ENSG00000158169 | FANCC | ENSG00000089225 | TBX5 |
| ENSG00000161960 | EIF4A1 | ENSG00000149428 | HYOU1 |
| ENSG00000181222 | POLR2A | ENSG00000166595 | FAM96B |
| ENSG00000165916 | PSMC3 | ENSG00000131462 | TUBG1 |
| ENSG00000198060 | MARCH5 | ENSG00000185990 | F8A3 |
| ENSG00000149923 | PPP4C | ENSG00000197932 | F8A1 |
| ENSG00000111667 | USP5 | ENSG00000198444 | F8A2 |
| ENSG00000198755 | RPL10A | ENSG00000031823 | RANBP3 |
| ENSG00000141499 | WRAP53 | ENSG00000100353 | EIF3D |
| ENSG00000093009 | CDC45 | ENSG00000163605 | PPP4R2 |
| ENSG00000105732 | ZNF574 | ENSG00000164162 | ANAPC10 |
| ENSG00000104064 | GABPB1 | ENSG00000132153 | DHX30 |
| ENSG00000108294 | PSMB3 | ENSG00000154723 | ATP5J |
| ENSG00000130856 | ZNF236 | ENSG00000182256 | GABRG3 |
| ENSG00000133980 | VRTN | ENSG00000119487 | MAPKAP1 |
| ENSG00000149308 | NPAT | ENSG00000132394 | EEFSEC |
| ENSG00000120071 | KANSL1 | ENSG00000122952 | ZWINT |
| ENSG00000129084 | PSMA1 | ENSG00000131042 | LILRB2 |
| ENSG00000117877 | CD3EAP | ENSG00000222004 | C7orf71 |
| ENSG00000127616 | SMARCA4 | ENSG00000168802 | CHTF8 |
| ENSG00000163882 | POLR2H | ENSG00000069849 | ATP1B3 |
| ENSG00000183718 | TRIM52 | ENSG00000074582 | BCS1L |
| ENSG00000106803 | SEC61B | ENSG00000103126 | AXIN1 |
| ENSG00000114942 | EEF1B2 | ENSG00000187144 | SPATA21 |
| ENSG00000067704 | IARS2 | ENSG00000221914 | PPP2R2A |
| ENSG00000114686 | MRPL3 | ENSG00000163386 | NBPF10 |
| ENSG00000172315 | TP53RK | ENSG00000134987 | WDR36 |
| ENSG00000173120 | KDM2A | ENSG00000132300 | PTCD3 |
| ENSG00000138442 | WDR12 | ENSG00000156931 | VPS8 |
| ENSG00000145982 | FARS2 | ENSG00000165632 | TAF3 |
| ENSG00000117481 | NSUN4 | ENSG00000044115 | CTNNA1 |
| ENSG00000142676 | RPL11 | ENSG00000035403 | VCL |
| ENSG00000164615 | CAMLG | ENSG00000088256 | GNA11 |
| ENSG00000138073 | PREB | ENSG00000164334 | FAM170A |
| ENSG00000136888 | ATP6V1G1 | ENSG00000166225 | FRS2 |
| ENSG00000221829 | FANCG | ENSG00000241186 | TDGF1 |
| ENSG00000198887 | SMC5 | ENSG00000196374 | HIST1H2BM |
| ENSG00000102900 | NUP93 | ENSG00000117614 | SYF2 |
| ENSG00000108344 | PSMD3 | ENSG00000154222 | CC2D1B |
| ENSG00000023191 | RNH1 | ENSG00000101367 | MAPRE1 |
| ENSG00000143621 | ILF2 | ENSG00000188186 | LAMTOR4 |
| ENSG00000112855 | HARS2 | ENSG00000166924 | NYAP1 |
| ENSG00000110536 | PTPMT1 | ENSG00000079805 | DNM2 |
| ENSG00000165629 | ATP5C1 | ENSG00000011260 | UTP18 |
| ENSG00000166847 | DCTN5 | ENSG00000089685 | BIRC5 |
| ENSG00000104852 | SNRNP70 | ENSG00000123908 | AGO2 |
| ENSG00000203814 | HIST2H2BF | ENSG00000057935 | MTA3 |
| ENSG00000009413 | REV3L | ENSG00000100811 | YY1 |
| ENSG00000130772 | MED18 | ENSG00000064102 | ASUN |
| ENSG00000079313 | REXO1 | ENSG00000006025 | OSBPL7 |
| ENSG00000012061 | ERCC1 | ENSG00000107372 | ZFAND5 |
| ENSG00000111642 | CHD4 | ENSG00000172922 | RNASEH2C |
| ENSG00000100462 | PRMT5 | ENSG00000075089 | ACTR6 |
| ENSG00000174100 | MRPL45 | ENSG00000165119 | HNRNPK |
| ENSG00000101421 | CHMP4B | ENSG00000182518 | FAM104B |
| ENSG00000144028 | SNRNP200 | ENSG00000041802 | LSG1 |
| ENSG00000108592 | FTSJ3 | ENSG00000206557 | TRIM71 |
| ENSG00000110048 | OSBP | ENSG00000124140 | SLC12A5 |
| ENSG00000147403 | RPL10 | ENSG00000063046 | EIF4B |
| ENSG00000198783 | ZNF830 | ENSG00000126581 | BECN1 |
| ENSG00000179409 | GEMIN4 | ENSG00000171530 | TBCA |
| ENSG00000147604 | RPL7 | ENSG00000206127 | GOLGA8O |
| ENSG00000136824 | SMC2 | ENSG00000167842 | MIS12 |
| ENSG00000104889 | RNASEH2A | ENSG00000033011 | ALG1 |
| ENSG00000146282 | RARS2 | ENSG00000146670 | CDCA5 |
| ENSG00000068784 | SRBD1 | ENSG00000198856 | OSTC |
| ENSG00000137822 | TUBGCP4 | ENSG00000111605 | CPSF6 |
| ENSG00000059691 | PET112 | ENSG00000087365 | SF3B2 |
| ENSG00000066827 | ZFAT | ENSG00000135845 | PIGC |
| ENSG00000148308 | GTF3C5 | ENSG00000100220 | RTCB |
| ENSG00000170185 | USP38 | ENSG00000131876 | SNRPA1 |
| ENSG00000160201 | U2AF1 | ENSG00000115392 | FANCL |
| ENSG00000141258 | SGSM2 | ENSG00000078618 | NRD1 |
| ENSG00000172660 | TAF15 | ENSG00000025770 | NCAPH2 |
| ENSG00000145833 | DDX46 | ENSG00000117682 | DHDDS |
| ENSG00000104980 | TIMM44 | ENSG00000198844 | ARHGEF15 |
| ENSG00000097046 | CDC7 | ENSG00000132603 | NIP7 |
| ENSG00000131368 | MRPS25 | ENSG00000162377 | SELRC1 |
| ENSG00000204209 | DAXX | ENSG00000137411 | VARS2 |
| ENSG00000129696 | TTI2 | ENSG00000064886 | CHI3L2 |
| ENSG00000108848 | LUC7L3 | ENSG00000137806 | NDUFAF1 |
| ENSG00000013573 | DDX11 | ENSG00000133030 | MPRIP |
| ENSG00000105248 | CCDC94 | ENSG00000136935 | GOLGA1 |
| ENSG00000183598 | HIST2H3D | ENSG00000243927 | MRPS6 |
| ENSG00000224226 | TBC1D3B | ENSG00000046647 | GEMIN8 |
| ENSG00000090470 | PDCD7 | ENSG00000133124 | IRS4 |
| ENSG00000031698 | SARS | ENSG00000255346 | NOX5 |
| ENSG00000108270 | AATF | ENSG00000103275 | UBE2I |
| ENSG00000159111 | MRPL10 | ENSG00000165502 | RPL36AL |
| ENSG00000149806 | FAU | ENSG00000100056 | DGCR14 |
| ENSG00000188739 | RBM34 | ENSG00000167972 | ABCA3 |
| ENSG00000152684 | PELO | ENSG00000053372 | MRTO4 |
| ENSG00000174374 | WBSCR16 | ENSG00000169813 | HNRNPF |
| ENSG00000107036 | KIAA1432 | ENSG00000198258 | UBL5 |
| ENSG00000204619 | PPP1R11 | ENSG00000103245 | NARFL |
| ENSG00000091651 | ORC6 | ENSG00000183513 | COA5 |
| ENSG00000134480 | CCNH | ENSG00000174547 | MRPL11 |
| ENSG00000164151 | KIAA0947 | ENSG00000173457 | PPP1R14B |
| ENSG00000164611 | PTTG1 | ENSG00000088038 | CNOT3 |
| ENSG00000111445 | RFC5 | ENSG00000115539 | PDCL3 |
| ENSG00000127481 | UBR4 | ENSG00000118181 | RPS25 |
| ENSG00000159352 | PSMD4 | ENSG00000160075 | SSU72 |
| ENSG00000137814 | HAUS2 | ENSG00000257949 | TEN1 |
| ENSG00000105220 | GPI | ENSG00000168028 | RPSA |
| ENSG00000140521 | POLG | ENSG00000213066 | FGFR1OP |
| ENSG00000075856 | SART3 | ENSG00000143228 | NUF2 |
| ENSG00000143742 | SRP9 | ENSG00000137413 | TAF8 |
| ENSG00000163029 | SMC6 | ENSG00000124207 | CSE1L |
| ENSG00000162227 | TAF6L | ENSG00000080815 | PSEN1 |
| ENSG00000100129 | EIF3L | ENSG00000132773 | TOE1 |
| ENSG00000170348 | TMED10 | ENSG00000129460 | NGDN |
| ENSG00000182217 | HIST2H4B | ENSG00000188613 | NANOS1 |
| ENSG00000183941 | HIST2H4A | ENSG00000163636 | PSMD6 |
| ENSG00000116221 | MRPL37 | ENSG00000146232 | NFKBIE |
| ENSG00000196235 | SUPT5H | ENSG00000135902 | CHRND |
| ENSG00000161920 | MED11 | ENSG00000143641 | GALNT2 |
| ENSG00000134690 | CDCA8 | ENSG00000073969 | NSF |
| ENSG00000131153 | GINS2 | ENSG00000041982 | TNC |
| ENSG00000138018 | EPT1 | ENSG00000108256 | NUFIP2 |
| ENSG00000173141 | MRP63 | ENSG00000198911 | SREBF2 |
| ENSG00000154727 | GABPA | ENSG00000141385 | AFG3L2 |
| ENSG00000120800 | UTP20 | ENSG00000176108 | CHMP6 |
| ENSG00000114767 | RRP9 | ENSG00000257365 | FNTB |
| ENSG00000174231 | PRPF8 | ENSG00000186487 | MYT1L |
| ENSG00000137547 | MRPL15 | ENSG00000127423 | AUNIP |
| ENSG00000146576 | C7orf26 | ENSG00000112110 | MRPL18 |
| ENSG00000065268 | WDR18 | ENSG00000114650 | SCAP |
| ENSG00000147162 | OGT | ENSG00000178104 | PDE4DIP |
| ENSG00000198917 | C9orf114 | ENSG00000105656 | ELL |
| ENSG00000180822 | PSMG4 | ENSG00000186393 | KRT26 |
| ENSG00000125977 | EIF2S2 | ENSG00000124541 | RRP36 |
| ENSG00000173418 | NAA20 | ENSG00000182108 | DEXI |
| ENSG00000155561 | NUP205 | ENSG00000139133 | ALG10 |
| ENSG00000173545 | ZNF622 | ENSG00000082068 | WDR70 |
| ENSG00000127993 | RBM48 | ENSG00000151388 | ADAMTS12 |
| ENSG00000197102 | DYNC1H1 | ENSG00000172172 | MRPL13 |
| ENSG00000119392 | GLE1 | ENSG00000184979 | USP18 |
| ENSG00000174444 | RPL4 | ENSG00000239857 | GET4 |
| ENSG00000149716 | ORAOV1 | ENSG00000069345 | DNAJA2 |
| ENSG00000155876 | RRAGA | ENSG00000073050 | XRCC1 |
| ENSG00000198841 | KTI12 | ENSG00000070985 | TRPM5 |
| ENSG00000056097 | ZFR | ENSG00000158715 | SLC45A3 |
| ENSG00000227057 | WDR46 | ENSG00000172062 | SMN1 |
| ENSG00000167670 | CHAF1A | ENSG00000205571 | SMN2 |
| ENSG00000127191 | TRAF2 | ENSG00000113141 | IK |
| ENSG00000072506 | HSD17B10 | ENSG00000186105 | LRRC70 |
| ENSG00000215021 | PHB2 | ENSG00000157895 | C12orf43 |
| ENSG00000175467 | SART1 | ENSG00000166441 | RPL27A |
| ENSG00000121073 | SLC35B1 | ENSG00000106346 | USP42 |
| ENSG00000079459 | FDFT1 | ENSG00000185379 | RAD51D |
| ENSG00000143493 | INTS7 | ENSG00000116667 | C1orf21 |
| ENSG00000141543 | EIF4A3 | ENSG00000176444 | CLK2 |
| ENSG00000174197 | MGA | ENSG00000105472 | CLEC11A |
| ENSG00000131269 | ABCB7 | ENSG00000065613 | SLK |
| ENSG00000089009 | RPL6 | ENSG00000005156 | LIG3 |
| ENSG00000197780 | TAF13 | ENSG00000125459 | MSTO1 |
| ENSG00000036549 | ZZZ3 | ENSG00000139146 | FAM60A |
| ENSG00000066135 | KDM4A | ENSG00000060069 | CTDP1 |
| ENSG00000176473 | WDR25 | ENSG00000130935 | NOL11 |
| ENSG00000124614 | RPS10 | ENSG00000115677 | HDLBP |
| ENSG00000107581 | EIF3A | ENSG00000105254 | TBCB |
| ENSG00000084463 | WBP11 | ENSG00000075539 | FRYL |
| ENSG00000137656 | BUD13 | ENSG00000196747 | HIST1H2AI |
| ENSG00000183751 | TBL3 | ENSG00000181513 | ACBD4 |
| ENSG00000119537 | KDSR | ENSG00000153107 | ANAPC1 |
| ENSG00000204220 | PFDN6 | ENSG00000160211 | G6PD |
| ENSG00000170291 | ELP5 | ENSG00000111481 | COPZ1 |
| ENSG00000198563 | DDX39B | ENSG00000070761 | C16orf80 |
| ENSG00000077549 | CAPZB | ENSG00000168924 | LETM1 |
| ENSG00000255529 | POLR2M | ENSG00000105058 | FAM32A |
| ENSG00000100034 | PPM1F | ENSG00000204569 | PPP1R10 |
| ENSG00000196367 | TRRAP | ENSG00000153914 | SREK1 |
| ENSG00000167258 | CDK12 | ENSG00000161509 | GRIN2C |
| ENSG00000039123 | SKIV2L2 | ENSG00000162702 | ZNF281 |
| ENSG00000076043 | REXO2 | ENSG00000004939 | SLC4A1 |
| ENSG00000213676 | ATF6B | ENSG00000139620 | KANSL2 |
| ENSG00000058453 | CROCC | ENSG00000025293 | PHF20 |
| ENSG00000153575 | TUBGCP5 | ENSG00000158545 | ZC3H18 |
| ENSG00000110700 | RPS13 | ENSG00000142546 | NOSIP |
| ENSG00000101181 | MTG2 | ENSG00000143398 | PIP5K1A |
| ENSG00000071539 | TRIP13 | ENSG00000197958 | RPL12 |
| ENSG00000075702 | WDR62 | ENSG00000067225 | PKM |
| ENSG00000171453 | POLR1C | ENSG00000172534 | HCFC1 |
| ENSG00000090989 | EXOC1 | ENSG00000155438 | MKI67IP |
| ENSG00000037897 | METTL1 | ENSG00000166582 | CENPV |
| ENSG00000095139 | ARCN1 | ENSG00000145912 | NHP2 |
| ENSG00000078142 | PIK3C3 | ENSG00000180992 | MRPL14 |
| ENSG00000141030 | COPS3 | ENSG00000118705 | RPN2 |
| ENSG00000126249 | PDCD2L | ENSG00000163161 | ERCC3 |
| ENSG00000117408 | IPO13 | ENSG00000136819 | C9orf78 |
| ENSG00000130725 | UBE2M | ENSG00000124787 | RPP40 |
| ENSG00000175054 | ATR | ENSG00000179104 | TMTC2 |
| ENSG00000149016 | TUT1 | ENSG00000140694 | PARN |
| ENSG00000165060 | FXN | ENSG00000143751 | SDE2 |
| ENSG00000117597 | DIEXF | ENSG00000136997 | MYC |
| ENSG00000185085 | INTS5 | ENSG00000147274 | RBMX |
| ENSG00000113595 | TRIM23 | ENSG00000084693 | AGBL5 |
| ENSG00000040633 | PHF23 | ENSG00000165271 | NOL6 |
| ENSG00000178952 | TUFM | ENSG00000221838 | AP4M1 |
| ENSG00000120539 | MASTL | ENSG00000171444 | MCC |
| ENSG00000103549 | RNF40 | ENSG00000101882 | NKAP |
| ENSG00000119723 | COQ6 | ENSG00000186847 | KRT14 |
| ENSG00000171311 | EXOSC1 | ENSG00000014824 | SLC30A9 |
| ENSG00000106245 | BUD31 | ENSG00000166685 | COG1 |
| ENSG00000118046 | STK11 | ENSG00000108349 | CASC3 |
| ENSG00000125484 | GTF3C4 | ENSG00000175216 | CKAP5 |
| ENSG00000089094 | KDM2B | ENSG00000259494 | MRPL46 |
| ENSG00000121621 | KIF18A | ENSG00000028310 | BRD9 |
| ENSG00000129911 | KLF16 | ENSG00000136450 | SRSF1 |
| ENSG00000102302 | FGD1 | ENSG00000204859 | ZBTB48 |
| ENSG00000135679 | MDM2 | ENSG00000165209 | STRBP |
| ENSG00000185115 | NDNL2 | ENSG00000163466 | ARPC2 |
| ENSG00000140553 | UNC45A | ENSG00000125485 | DDX31 |
| ENSG00000129562 | DAD1 | ENSG00000070778 | PTPN21 |
| ENSG00000100138 | NHP2L1 | ENSG00000126001 | CEP250 |
| ENSG00000111641 | NOP2 | ENSG00000169249 | ZRSR2 |
| ENSG00000173660 | UQCRH | ENSG00000111011 | RSRC2 |
| ENSG00000198677 | TTC37 | ENSG00000139496 | NUPL1 |
| ENSG00000135503 | ACVR1B | ENSG00000131746 | TNS4 |
| ENSG00000180998 | GPR137C | ENSG00000061936 | SFSWAP |
| ENSG00000153187 | HNRNPU | ENSG00000196584 | XRCC2 |
| ENSG00000106459 | NRF1 | ENSG00000168286 | THAP11 |
| ENSG00000156261 | CCT8 | ENSG00000119787 | ATL2 |
| ENSG00000118363 | SPCS2 | ENSG00000182446 | NPLOC4 |
| ENSG00000164134 | NAA15 | ENSG00000071462 | WBSCR22 |
| ENSG00000060642 | PIGV | ENSG00000213397 | HAUS7 |
| ENSG00000090889 | KIF4A | ENSG00000178028 | DMAP1 |
| ENSG00000101361 | NOP56 | ENSG00000067596 | DHX8 |
| ENSG00000167792 | NDUFV1 | ENSG00000198015 | MRPL42 |
| ENSG00000184162 | NR2C2AP | ENSG00000133706 | LARS |
| ENSG00000128524 | ATP6V1F | ENSG00000149635 | OCSTAMP |
| ENSG00000100387 | RBX1 | ENSG00000117505 | DR1 |
| ENSG00000110906 | KCTD10 | ENSG00000155868 | MED7 |
| ENSG00000147457 | CHMP7 | ENSG00000129197 | RPAIN |
| ENSG00000124570 | SERPINB6 | ENSG00000065978 | YBX1 |
| ENSG00000186468 | RPS23 | ENSG00000260238 | PMF1-BGLAP |
| ENSG00000136122 | BORA | ENSG00000178988 | MRFAP1L1 |
| ENSG00000047249 | ATP6V1H | ENSG00000168005 | C11orf84 |
| ENSG00000127804 | METTL16 | ENSG00000162408 | NOL9 |
| ENSG00000104412 | EMC2 | ENSG00000140350 | ANP32A |
| ENSG00000173726 | TOMM20 | ENSG00000261796 | ISY1-RAB43 |
| ENSG00000138777 | PPA2 | ENSG00000174405 | LIG4 |
| ENSG00000170043 | TRAPPC1 | ENSG00000197414 | GOLGA6L1 |
| ENSG00000124486 | USP9X | ENSG00000116062 | MSH6 |
| ENSG00000105705 | SUGP1 | ENSG00000116906 | GNPAT |
| ENSG00000223501 | VPS52 | ENSG00000134597 | RBMX2 |
| ENSG00000107815 | C10orf2 | ENSG00000071994 | PDCD2 |
| ENSG00000100109 | TFIP11 | ENSG00000112742 | TTK |
| ENSG00000136271 | DDX56 | ENSG00000106636 | YKT6 |
| ENSG00000146830 | GIGYF1 | ENSG00000101773 | RBBP8 |
| ENSG00000198382 | UVRAG | ENSG00000103061 | SLC7A6OS |
| ENSG00000160285 | LSS | ENSG00000140259 | MFAP1 |
| ENSG00000137770 | CTDSPL2 | ENSG00000197077 | KIAA1671 |
| ENSG00000116670 | MAD2L2 | ENSG00000204435 | CSNK2B |
| ENSG00000165280 | VCP | ENSG00000055130 | CUL1 |
| ENSG00000183963 | SMTN | ENSG00000100209 | HSCB |
| ENSG00000164961 | KIAA0196 | ENSG00000113048 | MRPS27 |
| ENSG00000157216 | SSBP3 | ENSG00000189403 | HMGB1 |
| ENSG00000129932 | DOHH | ENSG00000173011 | TADA2B |
| ENSG00000167721 | TSR1 | ENSG00000169836 | TACR3 |
| ENSG00000188352 | FOCAD | ENSG00000133816 | MICAL2 |
| ENSG00000104853 | CLPTM1 | ENSG00000141452 | C18orf8 |
| ENSG00000185883 | ATP6V0C | ENSG00000006715 | VPS41 |
| ENSG00000100519 | PSMC6 | ENSG00000136518 | ACTL6A |
| ENSG00000110107 | PRPF19 | ENSG00000100297 | MCM5 |
| ENSG00000184203 | PPP1R2 | ENSG00000165898 | ISCA2 |
| ENSG00000148824 | MTG1 | ENSG00000156384 | SFR1 |
| ENSG00000113810 | SMC4 | ENSG00000145414 | NAF1 |
| ENSG00000121152 | NCAPH | ENSG00000101972 | STAG2 |
| ENSG00000241127 | YAE1D1 | ENSG00000112658 | SRF |
| ENSG00000139197 | PEX5 | ENSG00000162736 | NCSTN |
| ENSG00000101464 | PIGU | ENSG00000103266 | STUB1 |
| ENSG00000132676 | DAP3 | ENSG00000008018 | PSMB1 |
| ENSG00000135972 | MRPS9 | ENSG00000149506 | ZP1 |
| ENSG00000089157 | RPLP0 | ENSG00000111530 | CAND1 |
| ENSG00000138035 | PNPT1 | ENSG00000027001 | MIPEP |
| ENSG00000171824 | EXOSC10 | ENSG00000152266 | PTH |
| ENSG00000153179 | RASSF3 | ENSG00000154174 | TOMM70A |
| ENSG00000110713 | NUP98 | ENSG00000164045 | CDC25A |
| ENSG00000100865 | CINP | ENSG00000164758 | MED30 |
| ENSG00000136045 | PWP1 | ENSG00000160401 | C9orf117 |
| ENSG00000167526 | RPL13 | ENSG00000155959 | VBP1 |
| ENSG00000088766 | CRLS1 | ENSG00000105409 | ATP1A3 |
| ENSG00000103510 | KAT8 | ENSG00000175106 | TVP23C |
| ENSG00000143368 | SF3B4 | ENSG00000185950 | IRS2 |
| ENSG00000156697 | UTP14A | ENSG00000149256 | TENM4 |
| ENSG00000176248 | ANAPC2 | ENSG00000116957 | TBCE |
| ENSG00000188786 | MTF1 | ENSG00000154719 | MRPL39 |
| ENSG00000175756 | AURKAIP1 | ENSG00000105364 | MRPL4 |
| ENSG00000140395 | WDR61 | ENSG00000198218 | QRICH1 |
| ENSG00000113368 | LMNB1 | ENSG00000013503 | POLR3B |
| ENSG00000060339 | CCAR1 | ENSG00000126756 | UXT |
| ENSG00000162385 | MAGOH | ENSG00000184988 | TMEM106A |
| ENSG00000105372 | RPS19 | ENSG00000186432 | KPNA4 |
| ENSG00000083312 | TNPO1 | ENSG00000156304 | SCAF4 |
| ENSG00000100142 | POLR2F | ENSG00000090565 | RAB11FIP3 |
| ENSG00000204560 | DHX16 | ENSG00000163508 | EOMES |
| ENSG00000197771 | MCMBP | ENSG00000147003 | TMEM27 |
| ENSG00000099817 | POLR2E | ENSG00000198730 | CTR9 |
| ENSG00000161980 | POLR3K | ENSG00000105321 | CCDC9 |
| ENSG00000117133 | RPF1 | ENSG00000120333 | MRPS14 |
| ENSG00000125901 | MRPS26 | ENSG00000121680 | PEX16 |
| ENSG00000168827 | GFM1 | ENSG00000088205 | DDX18 |
| ENSG00000161513 | FDXR | ENSG00000132432 | SEC61G |
| ENSG00000137818 | RPLP1 | ENSG00000186329 | TMEM212 |
| ENSG00000150990 | DHX37 | ENSG00000094804 | CDC6 |
| ENSG00000061794 | MRPS35 | ENSG00000169084 | DHRSX |
| ENSG00000143155 | TIPRL | ENSG00000107618 | RBP3 |
| ENSG00000253626 | EIF5AL1 | ENSG00000146426 | TIAM2 |
| ENSG00000231500 | RPS18 | ENSG00000198925 | ATG9A |
| ENSG00000188076 | SCGB1C1 | ENSG00000168242 | HIST1H2BI |
| ENSG00000174442 | ZWILCH | ENSG00000254772 | EEF1G |
| ENSG00000242028 | HYPK | ENSG00000090971 | NAT14 |
| ENSG00000124217 | MOCS3 | ENSG00000144381 | HSPD1 |
| ENSG00000134186 | PRPF38B | ENSG00000127774 | EMC6 |
| ENSG00000105849 | TWISTNB | ENSG00000126259 | KIRREL2 |
| ENSG00000137337 | MDC1 | ENSG00000111364 | DDX55 |
| ENSG00000132207 | SLX1A | ENSG00000100749 | VRK1 |
| ENSG00000181625 | SLX1B | ENSG00000159063 | ALG8 |
| ENSG00000110717 | NDUFS8 | ENSG00000163795 | ZNF513 |
| ENSG00000132341 | RAN | ENSG00000068394 | GPKOW |
| ENSG00000014123 | UFL1 | ENSG00000112659 | CUL9 |
| ENSG00000101191 | DIDO1 | ENSG00000187257 | RSBN1L |
| ENSG00000125952 | MAX | ENSG00000172167 | MTBP |
| ENSG00000163714 | U2SURP | ENSG00000176177 | ENTHD1 |
| ENSG00000253710 | ALG11 | ENSG00000166783 | KIAA0430 |
| ENSG00000104356 | POP1 | ENSG00000165006 | UBAP1 |
| ENSG00000130826 | DKC1 | ENSG00000188958 | UTS2B |
| ENSG00000198780 | FAM169A | ENSG00000136247 | ZDHHC4 |
| ENSG00000116688 | MFN2 | ENSG00000196363 | WDR5 |
| ENSG00000166166 | TRMT61A | ENSG00000116661 | FBXO2 |
| ENSG00000214517 | PPME1 | ENSG00000113013 | HSPA9 |
| ENSG00000077235 | GTF3C1 | ENSG00000090061 | CCNK |
| ENSG00000152240 | HAUS1 | ENSG00000051596 | THOC3 |
| ENSG00000063177 | RPL18 | ENSG00000140534 | TICRR |
| ENSG00000087157 | PGS1 | ENSG00000100216 | TOMM22 |
| ENSG00000100567 | PSMA3 | ENSG00000104613 | INTS10 |
| ENSG00000169371 | SNUPN | ENSG00000183474 | GTF2H2C |
| ENSG00000197651 | CCER1 | ENSG00000159128 | IFNGR2 |
| ENSG00000198900 | TOP1 | ENSG00000243725 | TTC4 |
| ENSG00000213551 | DNAJC9 | ENSG00000102898 | NUTF2 |
| ENSG00000152464 | RPP38 | ENSG00000170515 | PA2G4 |
| ENSG00000131467 | PSME3 | ENSG00000117036 | ETV3 |
| ENSG00000223510 | CDRT15 | ENSG00000196262 | PPIA |
| ENSG00000115053 | NCL | ENSG00000153037 | SRP19 |
| ENSG00000163041 | H3F3A | ENSG00000135801 | TAF5L |
| ENSG00000154813 | DPH3 | ENSG00000119414 | PPP6C |
| ENSG00000181873 | IBA57 | ENSG00000141013 | GAS8 |
| ENSG00000185591 | SP1 | ENSG00000113845 | TIMMDC1 |
| ENSG00000115355 | CCDC88A | ENSG00000175826 | CTDNEP1 |
| ENSG00000139350 | NEDD1 | ENSG00000117543 | DPH5 |
| ENSG00000108518 | PFN1 | ENSG00000204779 | FOXD4L5 |
| ENSG00000108264 | TADA2A | ENSG00000112249 | ASCC3 |
| ENSG00000134809 | TIMM10 | ENSG00000152256 | PDK1 |
| ENSG00000124383 | MPHOSPH10 | ENSG00000169217 | CD2BP2 |
| ENSG00000126067 | PSMB2 | ENSG00000166246 | C16orf71 |
| ENSG00000060688 | SNRNP40 | ENSG00000184164 | CRELD2 |
| ENSG00000042429 | MED17 | ENSG00000107960 | OBFC1 |
| ENSG00000196655 | TRAPPC4 | ENSG00000102384 | CENPI |
| ENSG00000107185 | RGP1 | ENSG00000079785 | DDX1 |
| ENSG00000124608 | AARS2 | ENSG00000133858 | ZFC3H1 |
| ENSG00000092098 | RNF31 | ENSG00000184110 | EIF3C |
| ENSG00000143569 | UBAP2L | ENSG00000146700 | SRCRB4D |
| ENSG00000233822 | HIST1H2BN | ENSG00000163380 | LMOD3 |
| ENSG00000171848 | RRM2 | ENSG00000116273 | PHF13 |
| ENSG00000183161 | FANCF | ENSG00000178229 | ZNF543 |
| ENSG00000166197 | NOLC1 | ENSG00000109475 | RPL34 |
| ENSG00000064703 | DDX20 | ENSG00000156469 | MTERFD1 |
| ENSG00000176102 | CSTF3 | ENSG00000155827 | RNF20 |
| ENSG00000106028 | SSBP1 | ENSG00000213741 | RPS29 |
| ENSG00000143315 | PIGM | ENSG00000165792 | METTL17 |
| ENSG00000136152 | COG3 | ENSG00000110844 | PRPF40B |
| ENSG00000134697 | GNL2 | ENSG00000100842 | EFS |
| ENSG00000159217 | IGF2BP1 | ENSG00000087495 | PHACTR3 |
| ENSG00000080608 | KIAA0020 | ENSG00000126261 | UBA2 |
| ENSG00000267368 | UPK3BL | ENSG00000136718 | IMP4 |
| ENSG00000130119 | GNL3L | ENSG00000091640 | SPAG7 |
| ENSG00000178950 | GAK | ENSG00000184886 | PIGW |
| ENSG00000205659 | LIN52 | ENSG00000184313 | MROH7 |
| ENSG00000123297 | TSFM | ENSG00000163481 | RNF25 |
| ENSG00000241370 | RPP21 | ENSG00000137054 | POLR1E |
| ENSG00000129351 | ILF3 | ENSG00000213085 | CCDC19 |
| ENSG00000174446 | SNAPC5 | ENSG00000171858 | RPS21 |
| ENSG00000132382 | MYBBP1A | ENSG00000130822 | PNCK |
| ENSG00000100664 | EIF5 | ENSG00000145216 | FIP1L1 |
| ENSG00000131469 | RPL27 | ENSG00000147130 | ZMYM3 |
| ENSG00000185128 | TBC1D3F | ENSG00000008086 | CDKL5 |
| ENSG00000111231 | GPN3 | ENSG00000165282 | PIGO |
| ENSG00000182774 | RPS17L | ENSG00000038358 | EDC4 |
| ENSG00000184779 | RPS17 | ENSG00000134684 | YARS |
| ENSG00000186871 | ERCC6L | ENSG00000153832 | FBXO36 |
| ENSG00000204568 | MRPS18B | ENSG00000140006 | WDR89 |
| ENSG00000108312 | UBTF | ENSG00000104643 | MTMR9 |
| ENSG00000167965 | MLST8 | ENSG00000151779 | NBAS |
| ENSG00000115241 | PPMIG | ENSG00000077348 | EXOSC5 |
| ENSG00000171103 | TRMT61B | ENSG00000131043 | AAR2 |
| ENSG00000116586 | LAMTOR2 | ENSG00000160193 | WDR4 |
| ENSG00000105793 | GTPBP10 | ENSG00000140691 | ARMC5 |
| ENSG00000100348 | TXN2 | ENSG00000141959 | PFKL |
| ENSG00000172757 | CFL1 | ENSG00000112053 | SLC26A8 |
| ENSG00000163634 | THOC7 | ENSG00000197111 | PCBP2 |
| ENSG00000008324 | SS18L2 | ENSG00000145191 | EIF2B5 |
| ENSG00000152404 | CWF19L2 | ENSG00000140988 | RPS2 |
| ENSG00000020129 | NCDN | ENSG00000181472 | ZBTB2 |

The gene symbols used in herein (including in Tables 3 and 4) are based on those found in the Human Gene Naming Committee (HGNC) which is searchable on the world-wide web at www.genenames.org. Ensembl IDs are provided for each gene symbol and are searchable world-wide web at www.ensembl.org.

The genes provided in Tables 3 and 4 are non-limiting examples of essential genes. Although additional essential genes will be apparent to the skilled artisan based on the knowledge in the art, the suitability of a particular gene for use according to the present disclosure can be determined, e.g., as discussed herein. For example, in some embodiments, a particular essential gene can be selected by analysis of potential off-target sites elsewhere in the genome. In some embodiments, only essential genes with one or more gRNA target sites that are unique in the human genome are selected for methods described herein. In some embodiments, only essential genes with one or more gRNA target sites that are found in only one other locus in the human genome are selected for methods described herein. In some embodiments, only essential genes with one or more gRNA target sites found in only two other loci in the human genome are selected for methods described herein.

Gene Product of Interest

The methods, systems and cells of the present disclosure enable the integration of a gene of interest at an essential gene of a cell. The gene of interest can encode any gene product of interest. In certain embodiments, a gene product of interest comprises an antibody, an antigen, an enzyme, a growth factor, a receptor (e.g., cell surface, cytoplasmic, or nuclear), a hormone, a lymphokine, a cytokine, a chemokine, a reporter, a functional fragment of any of the above, or a combination of any of the above.

In some embodiments, sequence for a gene product of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof; a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, a degradation signal, and the like.

In some embodiments, a gene product of interest may be but is not limited to, e.g., a therapeutic protein or a gene product that confers a desired feature to the modified cell. In some embodiments, the transgene encodes a reporter protein, such as a fluorescent protein (e.g., as described herein) and an enzyme (e.g., luciferase and lacZ). In some embodiments, a reporter gene may aid the tracking of therapeutic cells once they are introduced to a subject.

In some embodiments, a gene product of interest may be but is not limited to therapeutic proteins such as a protein deficient in a patient. In some embodiments, for example, therapeutic proteins include, but are not limited to, those deficient in lysosomal storage disorders, such as alpha-L-iduronidase, arylsulfatase A, beta-glucocerebrosidase, acid sphingomyelinase, and alpha- and beta-galactosidase; and those deficient in hemophilia such as Factor VIII and Factor IX. Other examples of therapeutic proteins include, but are not limited to, antibodies or antibody fragments (e.g., scFv) such as those targeting pathogenic proteins (e.g., tau, alpha-synuclein, and beta-amyloid protein) and those targeting cancer cells (e.g., chimeric antigen receptors (CAR) as described herein)

In some embodiments, a gene product of interest may be a protein involved in immune regulation, or an immunomodulatory protein. In some embodiments, for example, such proteins are, PD-L1, CTLA-4, M-CSF, IL-4, IL-6, IL-10, IL-11, IL-13, TGF-01, and various isoforms thereof. By way of example, in some embodiments, a gene product of interest may be an isoform of HLA-G (e.g., HLA-G1, -G2, -G3, -G4, -G5, -G6, or -G7) or HLA-E; allogeneic cells expressing such a nonclassical MHC class I molecule may be less immunogenic and better tolerated when transplanted into a human patient who is not the source of the cells, making “universal” cell therapy possible.

In some embodiments, an exemplary gene product of interest is one that confers therapeutic value, e.g., a new therapeutic activity to the cell. In some embodiments, exemplary gene products of interest are polypeptides such as a chimeric antigen receptor (CAR) or antigen-binding fragment thereof, a T cell receptor or antigen binding fragment thereof, a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof. It is to be understood that the methods and cells of the present disclosure are not limited to any particular gene product of interest and that the selection of a gene product of interest will depend on the type of cell and ultimate use of the cells.

In some embodiments, a gene product of interest may be a cytokine. In some embodiments, expression of a cytokine from a modified cell generated using a method as described herein allows for localized dosing of the cytokine in vivo (e.g., within a subject in need thereof) and/or avoids a need to systemically administer a high-dose of the cytokine to a subject in need thereof (e.g., a lower dose of the cytokine may be administered). In some embodiments, the risk of dose-limiting toxicities associated with administering a cytokine is reduced while cytokine mediated cell functions are maintained. In some embodiments, to facilitate cell function without the need to additionally administer high-doses of soluble cytokines, a partial or full peptide of one or more of IL2, IL4, IL6, IL7, IL9, IL10, IL11, IL12, IL15, IL18, IL21, IFN-α, IFN-β and/or their respective receptor is introduced to the cell to enable cytokine signaling with or without the expression of the cytokine itself, thereby maintaining or improving cell growth, proliferation, expansion, and/or effector function with reduced risk of cytokine toxicities. In some embodiments, the introduced cytokine and/or its respective native or modified receptor for cytokine signaling are expressed on the cell surface. In some embodiments, the cytokine signaling is constitutively activated. In some embodiments, the activation of the cytokine signaling is inducible. In some embodiments, the activation of the cytokine signaling is transient and/or temporal. In some embodiments, a gene product if interest can be IL2, IL3, IL4, IL6, IL7, IL9, IL10, IL11, IL12, IL13, IL15, IL21, GM-CSF, IFN-α, IFN-b, IFN-g, erythropoietin, and/or the respective cytokine receptor. In some embodiments, a gene product of interest can be CCL3, TNFα, CCL23, IL2RB, IL12RB2, or IRF7.

In some embodiments, a gene product of interest can be a chemokine and/or the respective chemokine receptor. In some embodiments, a chemokine receptor can be, but is not limited to, CCR2, CCR5, CCR8, CX3C1, CX3CR1, CXCR1, CXCR2, CXCR3A, CXCR3B, or CXCR2. In some embodiments, a chemokine can be, but is not limited to, CCL7, CCL19, or CXL14.

As used herein, the term “chimeric antigen receptor” or “CAR” refers to a receptor protein that has been modified to give cells expressing the CAR the new ability to target a specific protein. Within the context of the disclosure, a cell modified to comprise a CAR or an antigen binding fragment may be used for immunotherapy to target and destroy cells associated with a disease or disorder, e.g., cancer cells. In some embodiments, the CAR can bind to any antigen of interest.

CARs of interest can include, but are not limited to, a CAR targeting mesothelin, EGFR, HER2 and/or MICA/B. To date, mesothelin-targeted CAR T-cell therapy has shown early evidence of efficacy in a phase I clinical trial of subjects having mesothelioma, non-small cell lung cancer, and breast cancer (NCT02414269). Similarly, CARs targeting EGFR, HER2 and MICA/B have shown promise in early studies (see, e.g., Li et al. (2018), Cell Death & Disease, 9(177); Han et al. (2018) Am. J. Cancer Res., 8(1):106-119; and Demoulin 2017) Future Oncology, 13(8); the entire contents of each of which are expressly incorporated herein by reference in their entireties).

CARs are well-known to those of ordinary skill in the art and include those described in, for example: WO13/063419 (mesothelin), WO15/164594 (EGFR), WO13/063419 (HER2), WO16/154585 (MICA and MICB), the entire contents of each of which are expressly incorporated herein by reference in their entireties. In some embodiments, a gene product of interest is any suitable CAR, NK cell specific CAR (NK-CAR), T cell specific CAR, or other binder that targets a cell, e.g., an NK cell, to a target cell, e.g., a cell associated with a disease or disorder, may be expressed in the modified cells provided herein. Exemplary CARs, and binders, include, but are not limited to, bi-specific antigen binding CARs, switchable CARs, dimerizable CARs, split CARs, multi-chain CARs, inducible CARs, CARs and binders that bind BCMA, androgen receptor, PSMA, PSCA, Muc1, HPV viral peptides (i.e., E7), EBV viral peptides, WT1, CEA, EGFR, EGFRvIII, IL13Rα2, GD2, CA125, EpCAM, Muc16, carbonic anhydrase IX (CAIX), CCR1, CCR4, carcinoembryonic antigen (CEA), CD3, CD5, CD7, CD10, CD19, CD20, CD22, CD23, CD24, CD26, CD30, CD33, CD34, CD35, CD38 CD41, CD44, CD44V6, CD49f, CD56, CD70, CD92, CD99, CD123, CD133, CD135, CD148, CD150, CD261, CD362, CLEC12A, MDM2, CYP1B, livin, cyclin 1, NKp30, NKp46, DNAM1, NKp44, CA9, PD1, PDL1, an antigen of cytomegalovirus (CMV), epithelial glycoprotein-40 (EGP-40), GPRC5D, receptor tyrosine kinases erb-B2,3,4, EGFIR, ERBB folate binding protein (FBP), fetal acetylcholine receptor (AChR), folate receptor-a, ganglioside G3 (GD3) human Epidermal Growth Factor Receptor 2 (HER-2), human telomerase reverse transcriptase (hTERT), ICAM-1, Integrin B7, Interleukin-13 receptor subunit alpha-2 (IL-13Ra2), K-light chain, kinase insert domain receptor (KDR), Lewis A (CA19.9), Lewis Y (Le Y), L1 cell adhesion molecule (LI-CAM), LILRB2, melanoma antigen family A 1 (MAGE-A1), MICA/B, Mucin 16 (Muc-16), NKCSI, NKG2D ligands, c-Met, cancer-testis antigen NYES0-1, oncofetal antigen (h5T4), PRAME, prostate stem cell antigen (PSCA), PRAME prostate-specific membrane antigen (PSMA), tumor-associated glycoprotein 72 (TAG-72), TIM-3, TRBCI, TRBC2, vascular endothelial growth factor R2 (VEGF-R2), Wilms tumor protein (WT-1), a pathogen antigen, or any suitable combination thereof. Additional suitable CARs and binders for use in the modified cells provided herein will be apparent to those of skill in the art based on the present disclosure and the general knowledge in the art. Such additional suitable CARs include those described in FIG. 3 of Davies and Maher, Adoptive T-cell Immunotherapy of Cancer Using Chimeric Antigen Receptor-Grafted T Cells, Archivum Immunologiae et Therapiae Experimentalis 58(3):165-78 (2010), the entire contents of which are incorporated herein by reference. Additional CARs suitable for methods described herein include: CD171-specific CARs (Park et al., Mol Ther (2007) 15(4):825-833), EGFRvIII-specific CARs (Morgan et al, Hum Gene Ther (2012) 23(10): 1043-1053), EGF-R-specific CARs (Kobold et al, J Natl Cancer Inst (2014) 107 (0:364), carbonic anhydrase K-specific CARs (Lamers et al., Biochem Soc Trans (2016) 44(3):951-959), FR-a-specific CARs (Kershaw et al., Clin Cancer Res (2006) 12(20):6106-6015), HER2-specific CARs (Ahmed et al., J Clin Oncol (2015) 33(15) 1688-1696; Nakazawa et al., Mol Ther (2011) 19(12):2133-2143; Ahmed et al., Mol Ther (2009) 17(10): 1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21 (14):3149-3159), IL13Ra2-specific CARs (Brown et al., Clin Cancer Res (2015) 21(18):4062-4072), GD2-specific CARs (Louis et al., Blood (2011) 118(23):6050-6056; Caruana et al., Nat Med (2015) 21(5):524-529), ErbB2-specific CARs (Wilkie et al., J Clin Immunol (2012) 32(5): 1059-1070), VEGF-R-specific CARs (Chinnasamy et al., Cancer Res (2016) 22(2):436-447), FAP-specific CARs (Wang et al., Cancer Immunol Res (2014) 2(2): 154-166), MSLN-specific CARs (Moon et al., Clin Cancer Res (2011) 17(14):4719-30), CD19-specific CARs (Axicabtagene ciloleucel (Yescarta®) and Tisagenlecleucel (Kymriah®). See also, Li et al., J Hematol and Oncol (2018) 11(22), reviewing clinical trials of tumor-specific CARs.

As used herein, the term “CD16” refers to a receptor (FcγRIII) for the Fc portion of immunoglobulin G, and it is involved in the removal of antigen-antibody complexes from the circulation, as well as other antibody-dependent responses. In some embodiments, a CD16 protein is an hCD16 variant. In some embodiments an hCD16 variant is a high affinity F158V variant.

In some embodiments, a gene product of interest comprises a high affinity non-cleavable CD16 (hnCD16) or a variant thereof. In some embodiments, a high affinity non-cleavable CD16 or a variant thereof comprises at least any one of the followings: (a) F176V and S197P in ectodomain domain of CD16 (see e.g., Jing et al., Identification of an ADAM17 Cleavage Region in Human CD16 (FcγRIII) and the Engineering of a Non-Cleavable Version of the Receptor in NK Cells; PLOS One, 2015); (b) a full or partial ectodomain originated from CD64; (c) a non-native (or non-CD16) transmembrane domain; (d) a non-native (or non-CD16) intracellular domain; (e) a non-native (or nonCD16) signaling domain; (f) a non-native stimulatory domain; and (g) transmembrane, signaling, and stimulatory domains that are not originated from CD16, and are originated from a same or different polypeptide. In some embodiments, the non-native transmembrane domain is derived from CD3D, CD3E, CD3G, CD3s, CD4, CD5, CD5a, CD5b, CD27, CD2S, CD40, CDS4, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor (TCR) polypeptide. In some embodiments, the non-native stimulatory domain is derived from CD27, CD2S, 4-1BB, OX40, ICOS, PD-1, LAG-3, 2B4, BTLA, DAP1O, DAP12, CTLA-4, or NKG2D polypeptide. In some other embodiments, the non-native signaling domain is derived from CD3s, 2B4, DAP1O, DAP12, DNAM1, CD137 (41BB), IL21, IL7, IL12, IL15, NKp30, NKp44, NKp46, NKG2C, or NKG2D polypeptide. In some particular embodiments of a hnCD16 variant, the non-native transmembrane domain is derived from NKG2D, the non-native stimulatory domain is derived from 2B4, and the non-native signaling domain is derived from CD3s. In some embodiments, a gene product of interest comprises a high affinity cleavable CD16 (hnCD16) or a variant thereof. In some embodiments, a high affinity cleavable CD16 or a variant thereof comprises at least F176V. In some embodiments, a high affinity cleavable CD16 or a variant thereof does not comprise an S197P amino acid substitution.

As used herein, the term “IL-15/IL15RA” or “Interleukin-15” (IL-15) refers to a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells. IL-15 Receptor alpha (IL15RA) specifically binds IL-15 with very high affinity, and is capable of binding IL-15 independently of other subunits (see e.g., Mishra et al., Molecular pathways: Interleukin-15 signaling in health and in cancer, Clinical Cancer Research, 2014). It is suggested that this property allows IL-15 to be produced by one cell, endocytosed by another cell, and then presented to a third party cell. IL15RA is reported to enhance cell proliferation and expression of apoptosis inhibitor BCL2L1/BCL2-XL and BCL2. Exemplary sequences of IL-15 are provided in NG_029605.2, and exemplary sequences of IL-15RA are provided in NM_002189.4. In some embodiments, the IL-15R variant is a constitutively active IL-15R variant. In some embodiments, the constitutively active IL-15R variant is a fusion between IL-15R and an IL-15R agonist, e.g., an IL-15 protein or IL-15R-binding fragment thereof. In some embodiments, the IL-15R agonist is IL-15, or an IL-15R-binding variant thereof. Exemplary suitable IL-15R variants include, without limitation, those described, e.g., in Mortier E et al, 2006; The Journal of Biological Chemistry 2006 281: 1612-1619; or in Bessard-A et al., Mol Cancer Ther. 2009 September; 8(9):2736-45, the entire contents of each of which are incorporated by reference herein. In some embodiments, membrane bound trans-presentation of IL-15 is a more potent activation pathway than soluble IL-15 (see e.g., Imamura et al., Autonomous growth and increased cytotoxicity of natural killer cells expressing membrane-bound interleukin-15, Blood, 2014). In some embodiments, IL-15R expression comprises: IL15 and IL15Ra expression using a self-cleaving peptide; a fusion protein of IL15 and IL15Ra; an IL15/IL15Ra fusion protein with intracellular domain of IL15Ra truncated; a fusion protein of IL15 and membrane bound Sushi domain of IL15Ra; a fusion protein of IL15 and IL15Rβ; a fusion protein of IL15 and common receptor γC, wherein the common receptor γC is native or modified; and/or a homodimer of IL15Rβ.

As used herein, the term “IL-12” refers to interleukin-12, a cytokine that acts on T and natural killer cells. In some embodiments, a genetically engineered stem cell and/or progeny cell comprises a genetic modification that leads to expression of one or more of an interleukin 12 (IL12) pathway agonist, e.g., IL-12, interleukin 12 receptor (IL-12R) or a variant thereof (e.g., a constitutively active variant of IL-12R, e.g., an IL-12R fused to an IL-12R agonist (IL-12RA).

In some embodiments, the gene product of interest comprises a protein or polypeptide whose expression within a cell, e.g., a cell modified as described herein, enables the cell to inhibit or evade immune rejection after transplant or engraftment into a subject. In some embodiments, the gene product of interest is HLA-E, HLA-G, CTL4, CD47, or an associated ligand.

In some embodiments, the gene product of interest is a T cell receptor (TCR) or an antigen-binding fragment thereof, e.g., a recombinant TCR. In some embodiments, the recombinant TCR can bind to an antigen of interest, e.g., an antigen selected from, but not limited to, CD279, CD2, CD95, CD152, CD223CD272, TIM3, KIR, A2aR, SIRPa, CD200, CD200R, CD300, LPAS, NY-ESO, PD1, PDL1, or MAGE-A3/A6. In some embodiments, the TCR or antigen-binding fragment thereof can bind to a viral antigen, e.g., an antigen from hepatitis A, hepatitis B, hepatitis C (HCV), human papilloma virus (HPV) (e.g., HPV-16 (such as HPV-16 E6 or HPV-16 E7), HPV-18, HPV-31, HPV-33, or HPV-35), Epstein-Barr virus (EBV), human herpes virus 8 (HHV-8), human T-cell leukemia virus01 (HTLV-1), human T-cell leukemia virus-2 (HTLV-2) or a cytomegalovirus (CMV).

In some embodiments, the gene product of interest comprises a single-chain variable fragment that can bind to CD47, PD1, CTLA4, CD28, OX40, 4-1BB, and ligands thereof.

As used herein, the term “HLA-G” refers to the HLA non-classical class I heavy chain paralogues. This class I molecule is a heterodimer consisting of a heavy chain and a light chain (beta-2 microglobulin). The heavy chain is anchored in the membrane. HLA-G is expressed on fetal derived placental cells. HLA-G is a ligand for NK cell inhibitory receptor KIR2DL4, and therefore expression of this HLA by the trophoblast defends it against NK cell-mediated death. See e.g., Favier et al., Tolerogenic Function of Dimeric Forms of HLA-G Recombinant Proteins: A Comparative Study In Vivo PLOS One 2011, the entire contents of which are incorporated herein by reference. An exemplary sequence of HLA-G is set forth as NG_029039.1.

As used herein, the term “HLA-E” refers to the HLA class I histocompatibility antigen, alpha chain E, also sometimes referred to as MHC class I antigen E. The HLA-E protein in humans is encoded by the HLA-E gene. The human HLA-E is a non-classical MHC class I molecule that is characterized by a limited polymorphism and a lower cell surface expression than its classical paralogues. This class I molecule is a heterodimer consisting of a heavy chain and a light chain (beta-2 microglobulin). The heavy chain is anchored in the membrane. HLA-E binds a restricted subset of peptides derived from the leader peptides of other class I molecules. HLA-E expressing cells escape allogeneic responses and lysis by NK cells. See e.g., Geornalusse-G et al., Nature Biotechnology 2017 35 (8), the entire contents of which are incorporated herein by reference. Exemplary sequences of the HLA-E protein are provided in NM_005516.6.

As used herein, the term “CD47,” also sometimes referred to as “integrin associated protein” (IAP), refers to a transmembrane protein that in humans is encoded by the CD47 gene. CD47 belongs to the immunoglobulin superfamily, partners with membrane integrins, and also binds the ligands thrombospondin-1 (TSP-1) and signal-regulatory protein alpha (SIRPa). CD47 acts as a signal to macrophages that allows CD47-expressing cells to escape macrophage attack. See, e.g., Deuse-T, et al., Nature Biotechnology 2019 37: 252-258, the entire contents of which are incorporated herein by reference.

In some embodiments, a gene product of interest comprises a chimeric switch receptor (see e.g., WO2018094244A1—TGFBeta Signal Converter; Ankri et al., Human T cells Engineered to express a programmed death 1/28 costimulatory retargeting molecule display enhanced antitumor activity, The Journal of Immunology, Oct. 15, 2013, 191; Roth et al., Pooled knockin targeting for genome engineering of cellular immunotherapies, Cell. 2020 Apr. 30; 181(3):728-744.e21; and Boyerinas et al., A Novel TGF-β2/Interleukin Receptor Signal Conversion Platform That Protects CAR/TCR T Cells from TGF-β2-Mediated Immune Suppression and Induces T Cell Supportive Signaling Networks, Blood, 2017). In some embodiments, chimeric switch receptors are engineered cell-surface receptors comprising an extracellular domain from an endogenous cell-surface receptor and a heterologous intracellular signaling domain, such that ligand recognition by the extracellular domain results in activation of a different signaling cascade than that activated by the wild type form of the cell-surface receptor. In some embodiments, a chimeric switch receptor comprises an extracellular domain of an inhibitory cell-surface receptor fused to an intracellular domain that leads to the transmission of an activating signal rather than the inhibitory signal normally transduced by the inhibitory cell-surface receptor. In some embodiments, extracellular domains derived from cell-surface receptors known to inhibit immune effector cell activation can be fused to activating intracellular domains. In such an embodiment, engagement of the corresponding ligand may then activate signaling cascades that increase, rather than inhibit, the activation of the immune effector cell. For example, in some embodiments, a gene product of interest is a PD1-CD28 switch receptor, wherein the extracellular domain of PD1 is fused to the intracellular signaling domain of CD28 (See e.g.. Liu et al., Cancer Res 76:6 (2016), 1578-1590 and Moon et al., Molecular Therapy 22 (2014), S201). In some embodiments, encoding gene product of interest is or comprises the extracellular domain of CD200R and the intracellular signaling domain of CD28 (See Oda et al., Blood 130:22 (2017), 2410-2419).

In some embodiments, a gene product of interest is a reporter gene (e.g., GFP, mCherry, etc.). In some embodiments, a reporter gene is utilized to confirm the suitability of a knock-in cassette's expression capacity. In certain embodiments, a gene product of interest may be a colored or fluorescent protein such as: blue/UV proteins, e.g. TagBFP, mTagBFP2, Azurite, EBFP2, mKalamal, Sirius, Sapphire, T-Sapphire; cyan proteins, e.g. ECFP, Cerulean, SCFP3A, mTurquoise, mTurquoise2, monomeric Midoriishi-Cyan, TagCFP, mTFP1; green proteins, e.g. EGFP, Emerald, Superfolder GFP, Monomeric Azami Green, TagGFP2, mUKG, m Wasabi, Clover, mNeonGreen; yellow proteins, e.g. EYFP, Citrine, Venus, SYFP2, TagYFP; orange proteins, e.g. Monomeric Kusabira-Orange, mKOK, mK02, mOrange, m0range2; red proteins, e.g. mRaspberry, mStrawberry, mTangerine, tdTomato, TagRFP, TagRFP-T, mApple, mRuby, mRuby2; far-red proteins, e.g. mPlum, HcRed-Tandem, mKate2, mNeptune, NirFP; near-IR proteins, e.g. TagRFP657, IFP1.4, iRFP; long stokes shift proteins, e.g. mKeima Red, LSS-mKatel, LSS-mKate2, mBeRFP; photoactivatible proteins, e.g. PA-GFP, PAmCherryl, PATagRFP; photoconvertible proteins, e.g. Kaede (green), Kaede (red), KikGR1 (green), KikGR1 (red), PS-CFP2, PS-CFP2, mEos2 (green), mEos2 (red), mEos3.2 (green), mEos3.2 (red), PSmOrange, PSmOrange, photoswitchable proteins, e.g. Dronpa, and combinations thereof.

In some embodiments, a gene of interest provided herein can optionally include a sequence encoding a destabilizing domain (“a destabilizing sequence”) for temporal and/or spatial control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (MA), and immunohistochemistry).

Additional examples of destabilizing sequences are known in the art. In some embodiments, the destabilizing sequence is a FK506- and rapamycin-binding protein (FKBP12) sequence, and the stabilizing ligand is Shield-1 (Sh1d1) (Banaszynski et al. (2012) Cell 126(5): 995-1004, which is incorporated in its entirety herein by reference). In some embodiments, a destabilizing sequence is a DHFR sequence, and a stabilizing ligand is trimethoprim (TMP) (Iwamoto et al. (2010) Chem Biol 17:981-988, which is incorporated in its entirety herein by reference). In some embodiments, a destabilizing domain is small molecule-assisted shutoff (SMASh), where a constitutive degron with a protease and its corresponding cleavage site derived from hepatitis C virus are combined. In some embodiments, a destabilizing domain comprises a HaloTag system, dTag system, and/or nanobody (see e.g., Luh et al., Prey for the proteasome: targeted protein degradation—a medicinal chemist's perspective; Angewandte Chemie, 2020).

In some embodiments, a destabilizing sequence can be used to temporally control a cell modified as described herein.

In some embodiments, a gene product of interest may be a suicide gene, (see e.g., Zarogoulidis et al., Suicide Gene Therapy for Cancer—Current Strategies; J Genet Syndr Gene Ther. 2013). In some embodiments, a suicide gene can use a gene-directed enzyme prodrug therapy (GDEPT) approach, a dimerization inducing approach, and/or therapeutic monoclonal antibody mediated approach. In some embodiments, a suicide gene is biologically inert, has an adequate bio-availability profile, an adequate bio-distribution profile, and can be characterized by intrinsic acceptable and/or absence of toxicity. In some embodiments, a suicide gene codes for a protein able to convert, at a cellular level, a non-toxic prodrug into a toxic product. In some embodiments, a suicide gene may improve the safety profile of a cell described herein (see e.g., Greco et al., Improving the safety of cell therapy with the TK-suicide gene; Front Pharmacology. 2015; Jones et al., Improving the safety of cell therapy products by suicide gene transfer; Frontiers Pharmacology, 2014). In some embodiments, a suicide gene is a herpes simplex virus thymidine kinase (HSV-TK). In some embodiments, a suicide gene is a cytosine deaminase (CD). In some embodiments, a suicide gene is an apoptotic gene (e.g., a caspase). In some embodiments, a suicide gene is dimerization inducing, e.g., comprising an inducible FAS (iFAS) or inducible Caspase9 (iCasp9)/AP1903 system. In some embodiments, a suicide gene is a CD20 antigen, and cells expressing such an antigen can be eliminated by clinical-grade anti-CD20 antibody administration. In some embodiments, a suicide gene is a truncated human EGFR polypeptide (huEGFRt) which confers sensitivity to a pharmaceutical-grade anti-EGFR monoclonal antibody, e.g., cetuximab. In some embodiments a suicide gene is a c-myc tag, which confers sensitivity to pharmaceutical-grade anti-cmyc antibodies.

In some embodiments, a gene product of interest may be a safety switch signal. In cell therapy, a safety switch can be used to stop proliferation of the genetically modified cells when their presence in the patient is not desired, for example, if the cells do not function properly, if planned therapeutic interventions change, or if the therapeutic goal has been achieved. In some embodiments, a safety switch may, for example, be a so-called suicide gene, or suicide switch, which upon administration of a pharmaceutical compound to the patient, will be activated or inactivated such that the cells enter apoptosis. Suicide genes, sometimes called suicide switches or safety switches can be triggered or activated by a cellular event, environmental event or chemical agent resulting in a cellular response by cells that have the suicide gene incorporated in their genome. In some embodiments, activation of a safety switch induces cellular apoptosis. In some embodiments, activation of the safety switch inhibits growth of cells incorporated with the safety switch. In some embodiments, a suicide switch may encode an enzyme not found in humans (e.g., a bacterial or viral enzyme) that converts a harmless substance into a toxic metabolite in the human cell. Examples of suicide switch include, without limitation, genes for thymidine kinases, cytosine deaminases, intracellular antibodies, telomerases, toxins, caspases (e.g., iCaspase9) and HSV-TK, and DNases. In some embodiments, the suicide gene may be a thymidine kinase (TK) gene from the Herpes Simplex Virus (HSV) and the suicide TK gene becomes toxic to the cell upon administration of ganciclovir, valganciclovir, famciclovir, or the like to the patient.

In some embodiments, a safety switch may be a rapamycin-inducible human Caspase 9-based (RapaCasp9) cellular suicide switch in which a truncated caspase 9 gene, which has its CARD domain removed, is linked after either the FRB (FKBP12-rapamycin binding) domain of mTOR, or FKBP12 (FK506-binding protein 12). Addition of the drug rapamycin enables heterodimerization of FRB and FKBP12 which subsequently causes homodimerization of truncated caspase 9 and induction of apoptosis. In some embodiments, using a two construct and/or biallelic approach as described herein, FRB and FKBP12 are separated onto different alleles by incorporating two donor constructs, one with one or more transgenes plus FRB, the other with one or more transgenes plus FKBP12. When referring to a safety switch in this application, it should be interpreted to include all components necessary for the function of the safety switch (e.g., FRB domain and FKBP12 domain and truncated caspase 9 gene are all components of, and make up, the safety switch).

Exemplary DHFR destabilizing amino acid sequence
SEQ ID NO: 160
MISLIAALAVDYVIGMENAMPWNLPADLAWFKRNTLNKPVIMGRHTWESIGRPLPGRKNIILSS
QPSTDDRVTWVKSVDEAIAACGDVPEIMVIGGGRVIEQFLPKAQKLYLTHIDAEVEGDTHFPDY
EPDDWESVFSEFHDADAQNSHSYCFEILERR
Exemplary DHFR destabilizing nucleotide sequence
SEQ ID NO: 161
GGTACCATCAGTCTGATTGCGGCGTTAGCGGTAGATTACGTTATCGGCATGGAAAACGCCATGC
CGTGGAACCTGCCTGCCGATCTCGCCTGGTTTAAACGCAACACCTTAAATAAACCCGTGATTAT
GGGCCGCCATACCTGGGAATCAATCGGTCGTCCGTTGCCAGGACGCAAAAATATTATCCTCAGC
AGTCAACCGAGTACGGACGATCGCGTAACGTGGGTGAAGTCGGTGGATGAAGCCATCGCGGCGT
GTGGTGACGTACCAGAAATCATGGTGATTGGCGGCGGTCGCGTTATTGAACAGTTCTTGCCAAA
AGCGCAAAAACTGTATCTGACGCATATCGACGCAGAAGTGGAAGGCGACACCCATTTCCCGGAT
TACGAGCCGGATGACTGGGAATCGGTATTCAGCGAATTCCACGATGCTGATGCGCAGAACTCTC
ACAGCTATTGCTTTGAGATTCTGGAGCGGCGATAA
Exemplary destabilizing domain
SEQ ID NO: 162
ATCAGTCTGATTGCGGCGTTAGCGGTAGATTACGTTATCGGCATGGAAAACGCCATGCCGTGGA
ACCTGCCTGCCGATCTCGCCTGGTTTAAACGCAACACCTTAAATAAACCCGTGATTATGGGCCG
CCATACCTGGGAATCAATCGGTCGTCCGTTGCCAGGACGCAAAAATATTATCCTCAGCAGTCAA
CCGAGTACGGACGATCGCGTAACGTGGGTGAAGTCGGTGGATGAAGCCATCGCGGCGTGTGGTG
ACGTACCAGAAATCATGGTGATTGGCGGCGGTCGCGTTATTGAACAGTTCTTGCCAAAAGCGCA
AAAACTGTATCTGACGCATATCGACGCAGAAGTGGAAGGCGACACCCATTTCCCGGATTACGAG
CCGGATGACTGGGAATCGGTATTCAGCGAATTCCACGATGCTGATGCGCAGAACTCTCACAGCT
ATTGCTTTGAGATTCTGGAGCGGCGA
Exemplary FKBP12 destabilizing peptide amino acid sequence
SEQ ID NO: 163
MGVEKQVIRPGNGPKPAPGQTVTVHCTGFGKDGDLSQKFWSTKDEGQKPFSFQIGKGAVIKGWD
EGVIGMQIGEVARLRCSSDYAYGAGGFPAWGIQPNSVLDFEIEVLSVQ

In some embodiments, a coding sequence for a single gene product of interest may be included in a knock-in cassette. In some embodiments, coding sequences for two gene products of interest may be included in a single knock-in cassette; in some embodiments, this may be referred to as a bicistronic or multicistronic construct. In some embodiments, coding sequences for more than two gene products of interest may be included in a single knock-in cassette; in some embodiments, this may be referred to as a multicistronic construct. In some embodiments, when more than one coding sequence for more than one gene product of interest is included in a knock-in cassette, these sequences may have a linker sequence connecting them. Linker sequences are generally known in the art, an exemplary linker sequence is identified in SEQ ID NO: 164. In some embodiments, where more than one coding sequence for more than one gene product of interest is included in a knock-in cassette, these sequences may be connected by a linker sequence, an IRES, and/or 2A element.

In some embodiments, an oligonucleotide encoding a gene product of interest comprises or consists of the sequence of any one of SEQ ID NOs: 161, 162, or 164-182. In some embodiments, a gene product of interest comprises or consists of a sequence that is at least 85%, 90%, 95%, 98% or 99% identical to any one of SEQ ID NOs: 161, 162, or 164-182.

Exemplary linker sequence
SEQ ID NO: 164
TCTGGCGGAGGAAGCGGAGGCGGAGGATCTGGTGGTGGTGGATCTGGCGGCGGTGGTAGTGGCG
GAGGTTCTCTGCAA
exemplary CD16 knock-in cassette sequence
SEQ ID NO: 165
ATGTGGCAACTGCTGCTGCCTACAGCTCTGCTGCTTCTGGTGTCTGCCGGCATGAGAACCGAGG
ATCTGCCTAAGGCCGTGGTGTTCCTGGAACCTCAGTGGTACAGAGTGCTGGAAAAGGACAGCGT
GACCCTGAAGTGCCAGGGCGCCTATTCTCCCGAGGACAATAGCACCCAGTGGTTCCACAACGAG
AGCCTGATCAGCAGCCAGGCCAGCAGCTACTTTATCGATGCCGCCACCGTGGACGACAGCGGCG
AGTACAGATGCCAGACCAATCTGAGCACCCTGAGCGACCCTGTGCAGCTGGAAGTGCACATTGG
ATGGTTGCTGCTGCAAGCCCCTAGATGGGTGTTCAAAGAAGAGGACCCCATCCACCTGAGATGC
CACTCTTGGAAGAACACAGCCCTGCACAAAGTGACCTACCTGCAGAACGGCAAGGGCAGAAAGT
ACTTCCACCACAACAGCGACTTCTACATCCCCAAGGCCACACTGAAGGACTCCGGCTCCTACTT
CTGCAGAGGCCTGGTCGGCAGCAAGAACGTGTCCAGCGAGACAGTGAACATCACCATCACACAG
GGCCTCGCCGTGTCTACCATCAGCAGCTTTTTCCCACCTGGCTATCAGGTGTCCTTCTGCCTGG
TCATGGTGCTGCTGTTCGCCGTGGATACCGGCCTGTACTTCAGCGTCAAGACCAACATCCGGTC
CAGCACCAGAGACTGGAAGGACCACAAGTTCAAGTGGCGGAAGGACCCTCAGGACAAGTAA
exemplary CD16 knock-in cassette sequence
SEQ ID NO: 166
ATGTGGCAGCTGTTGCTGCCGACAGCCCTCCTGTTGCTGGTCTCCGCTGGCATGAGAACCGAGG
ATCTGCCTAAGGCCGTGGTGTTCCTGGAACCTCAGTGGTACAGAGTGCTGGAAAAGGACAGCGT
GACCCTGAAGTGCCAGGGCGCCTATTCTCCCGAGGACAATAGCACCCAGTGGTTCCACAACGAG
AGCCTGATCAGCAGCCAGGCCAGCAGCTACTTTATCGATGCCGCCACCGTGGACGACAGCGGCG
AGTACAGATGCCAGACCAATCTGAGCACCCTGAGCGACCCTGTGCAGCTGGAAGTGCACATTGG
ATGGTTGCTGCTGCAAGCCCCTAGATGGGTGTTCAAAGAAGAGGACCCCATCCACCTGAGATGC
CACTCTTGGAAGAACACAGCCCTGCACAAAGTGACCTACCTGCAGAACGGCAAGGGCAGAAAGT
ACTTCCACCACAACAGCGACTTCTACATCCCCAAGGCCACACTGAAGGACTCCGGCTCCTACTT
CTGCAGAGGCCTGGTCGGCAGCAAGAACGTGTCCAGCGAGACAGTGAACATCACCATCACACAG
GGCCTCGCCGTGTCTACCATCAGCAGCTTTTTCCCACCTGGCTATCAGGTGTCCTTCTGCCTGG
TCATGGTGCTGCTGTTCGCCGTGGATACCGGCCTGTACTTCAGCGTCAAGACCAACATCCGGTC
CAGCACCAGAGACTGGAAGGACCACAAGTTCAAGTGGCGGAAGGACCCTCAGGACAAG
exemplary CD47 knock-in cassette sequence
SEQ ID NO: 167
ATGTGGCCCCTGGTAGCGGCGCTGTTGCTGGGCTCGGCGTGCTGCGGATCAGCTCAGCTACTAT
TTAATAAAACAAAATCTGTAGAATTCACGTTTTGTAATGACACTGTCGTCATTCCATGCTTTGT
TAGTAATATGGAGGCACAAAACACTAGTGAAGTATACGTAAAGTGGAAATTTAAAGGAAGAGAT
ATTTACACCTTTGATGGAGCTCTAAACAAGTCCACTGTCCCCACTGACTTTAGTAGTGCAAAAA
TTGAAGTCTCACAATTAGTAAAAGGAGATGCCTCTTTGAAGATGGATAAGAGTGATGCTGTCTC
ACACACAGGAAACTAGACTTGTGAAGTAACAGAATTAACCAGAGAAGGTGAAACGATCATCGAG
CTAAAATATCGTGTTGTTTCATGGTTTTCTCCAAATGAAAATATTCTTATTGTTATTTTCCCAA
TTTTTGCTATACTCCTGTTCTGGGGACAGTTTGGTATTAAAACACTTAAATATAGATCCGGTGG
TATGGATGAGAAAACAATTGCTTTACTTGTTGCTGGACTAGTGATCACTGTCATTGTCATTGTT
GGAGCCATTCTTTTCGTCCCAGGTGAATATTCATTAAAGAATGCTACTGGCCTTGGTTTAATTG
TGAGTTCTACAGGGATATTAATATTAGTTGAGTAGTATGTGTTTAGTACAGCGATTGGATTAAC
CTCCTTCGTCATTGCCATATTGGTTATTCAGGTGATAGCCTATATCCTCGCTGTGGTTGGACTG
AGTCTCTGTATTGCGGCGTGTATACCAATGCATGGCCCTCTTCTGATTTCAGGTTTGAGTATCT
TAGCTCTAGCACAATTAGTTGGACTAGTTTATATGAAATTTGTGGCTTCCAATCAGAAGACTAT
ACAACCTCCTAGGAAAGCTGTAGAGGAACCCCTTAATGCATTCAAAGAATCAAAAGGAATGATG
AATGATGAATGA
exemplary IL15 knock-in cassette sequence
SEQ ID NO: 168
AATTGGGTCAACGTGATCAGCGACCTGAAGAAGATCGAGGACCTGATCCAGAGCATGCACATCG
ACGCCACACTGTACACCGAGTCCGATGTGCACCCTAGCTGCAAAGTGACCGCCATGAAGTGCTT
TCTGCTGGAACTGCAAGTGATCAGCCTGGAAAGCGGCGACGCCAGCATCCACGATACCGTGGAA
AACCTGATCATCCTGGCCAACAACAGCCTGAGCAGCAACGGCAATGTGACCGAGAGCGGCTGCA
AAGAGTGCGAGGAACTGGAAGAGAAGAACATCAAAGAGTTCCTCCAGAGCTTCGTCCACATCGT
GCAGATGTTCATCAACACCAGC
exemplary IgE-IL15 knock-in cassette sequence
SEQ ID NO: 169
ATGGATTGGACCTGGATCCTGTTTCTGGTGGCCGCTGCCACAAGAGTGCACAGCAATTGGGTCA
ACGTGATCAGCGACCTGAAGAAGATCGAGGACCTGATCCAGAGCATGCACATCGACGCCACACT
GTACACCGAGTCCGATGTGCACCCTAGCTGCAAAGTGACCGCCATGAAGTGCTTTCTGCTGGAA
CTGCAAGTGATCAGCCTGGAAAGCGGCGACGCCAGCATCCACGATACCGTGGAAAACCTGATCA
TCCTGGCCAACAACAGCCTGAGCAGCAACGGCAATGTGACCGAGAGCGGCTGCAAAGAGTGCGA
GGAACTGGAAGAGAAGAACATCAAAGAGTTCCTCCAGAGCTTCGTCCACATCGTGCAGATGTTC
ATCAACACCAGC
exemplary IgE-IL15 pro-peptide cargo sequence
SEQ ID NO: 170
ATGGACTGGACCTGGATTCTGTTCCTGGTCGCGGCTGCAACGCGAGTCCATAGCGGTATCCATG
TTTTTATTCTTGGGTGTTTTTCTGCTGGGCTGCCTAAGACCGAGGCCAACTGGGTAAATGTCAT
CAGTGACCTCAAGAAAATAGAAGACCTTATACAAAGCATGCACATTGATGCTACTCTCTACACT
GAGTCAGATGTACATCCCTCATGCAAAGTGACGGCCATGAAATGTTTCCTCCTCGAACTTCAAG
TCATATCTCTGGAAAGTGGCGACGCGTCCATCCACGACACGGTCGAAAACCTGATAATACTCGC
TAATAATAGTCTCTCTTCAAATGGTAACGTAACCGAGTCAGGTTGCAAAGAGTGCGAAGAGTTG
GAAGAAAAAAACATAAAGGAGTTCCTGCAAAGTTTCGTGCACATTGTGCAGATGTTCATTAATA
CCTCT
exemplary IL15Rα cargo sequence
SEQ ID NO: 171
ATCACCTGTCCTCCACCTATGAGCGTGGAACACGCCGACATCTGGGTCAAGAGCTACAGCCTGT
ACAGCAGAGAGCGGTACATCTGCAACAGCGGCTTCAAGAGAAAGGCCGGCACAAGCAGCCTGAC
CGAGTGTGTGCTGAACAAGGCCACAAACGTGGCCCACTGGACCACACCTAGCCTGAAGTGCATC
AGAGATCCCGCTCTGGTTCATCAGAGGCCTGCCCCTCCATCTACAGTGACAACAGCTGGCGTGA
CCCCTCAGCCTGAGTCTCTGTCTCCATCTGGAAAAGAGCCTGCCGCCAGCTCTCCCAGCTCTAA
CAATACTGCTGCCACCACAGCCGCTATCGTGCCTGGATCTCAGCTGATGCCTAGCAAGAGCCCT
AGCACCGGCACAACAGAGATCAGCTCTCACGAGAGCAGCCACGGAACACCTTCTCAGACCACCG
CCAAGAATTGGGAGCTGACAGCCTCTGCCTCTCATCAGCCACCTGGCGTGTACCCACAGGGCCA
CTCTGATACAACAGTGGCCATCAGCACCAGCACCGTTCTGCTGTGTGGCCTGTCTGCTGTTAGC
CTGCTGGCCTGCTACCTGAAGTCTAGACAGACACCTCCTCTGGCCAGCGTGGAAATGGAAGCCA
TGGAAGCTCTGCCTGTCACATGGGGCACCAGCAGCAGAGATGAGGACCTCGAGAATTGCAGCCA
CCACCTG
exemplary mbIL-15 cargo sequence
SEQ ID NO: 172
ATGGATTGGACCTGGATCCTGTTTCTGGTGGCCGCTGCCACAAGAGTGCACAGCAATTGGGTCA
ACGTGATCAGCGACCTGAAGAAGATCGAGGACCTGATCCAGAGCATGCACATCGACGCCACACT
GTACACCGAGTCCGATGTGCACCCTAGCTGCAAAGTGACCGCCATGAAGTGCTTTCTGCTGGAA
CTGCAAGTGATCAGCCTGGAAAGCGGCGACGCCAGCATCCACGATACCGTGGAAAACCTGATCA
TCCTGGCCAACAACAGCCTGAGCAGCAACGGCAATGTGACCGAGAGCGGCTGCAAAGAGTGCGA
GGAACTGGAAGAGAAGAACATCAAAGAGTTCCTCCAGAGCTTCGTCCACATCGTGCAGATGTTC
ATCAACACCAGCTCTGGCGGAGGAAGCGGAGGCGGAGGATCTGGTGGTGGTGGATCTGGCGGCG
GTGGTAGTGGCGGAGGTTCTCTGCAAATCACCTGTCCTCCACCTATGAGCGTGGAACACGCCGA
CATCTGGGTCAAGAGCTACAGCCTGTACAGCAGAGAGCGGTACATCTGCAACAGCGGCTTCAAG
AGAAAGGCCGGCACAAGCAGCCTGACCGAGTGTGTGCTGAACAAGGCCACAAACGTGGCCCACT
GGACCACACCTAGCCTGAAGTGCATCAGAGATCCCGCTCTGGTTCATCAGAGGCCTGCCCCTCC
ATCTACAGTGACAACAGCTGGCGTGACCCCTCAGCCTGAGTCTCTGTCTCCATCTGGAAAAGAG
CCTGCCGCCAGCTCTCCCAGCTCTAACAATACTGCTGCCACCACAGCCGCTATCGTGCCTGGAT
CTCAGCTGATGCCTAGCAAGAGCCCTAGCACCGGCACAACAGAGATCAGCTCTCACGAGAGCAG
CCACGGAACACCTTCTCAGACCACCGCCAAGAATTGGGAGCTGACAGCCTCTGCCTCTCATCAG
CCACCTGGCGTGTACCCACAGGGCCACTCTGATACAACAGTGGCCATCAGCACCAGCACCGTTC
TGCTGTGTGGCCTGTCTGCTGTTAGCCTGCTGGCCTGCTACCTGAAGTCTAGACAGACACCTCC
TCTGGCCAGCGTGGAAATGGAAGCCATGGAAGCTCTGCCTGTCACATGGGGCACCAGCAGCAGA
GATGAGGAGCTCGAGAATTGGAGCCACCACCTG
exemplary mbIL-15 cargo sequence
SEQ ID NO: 173
ATGGACTGGACCTGGATTCTGTTCCTGGTCGCGGCTGCAACGCGAGTCCATAGCGGTATCCATG
TTTTTATTCTTGGGTGTTTTTCTGCTGGGCTGCCTAAGACCGAGGCCAACTGGGTAAATGTCAT
GAGTGAGCTCAAGAAAATAGAAGACCTTATACAAAGCATGCACATTGATGCTAGTCTCTACACT
GAGTCAGATGTACATCCCTCATGCAAAGTGACGGCCATGAAATGTTTCCTCCTCGAACTTCAAG
TCATATCTCTGGAAAGTGGCGACGCGTCCATCCACGACACGGTCGAAAACCTGATAATACTCGC
TAATAATAGTCTCTCTTCAAATGGTAACGTAACCGAGTGAGGTTGCAAAGAGTGCGAAGAGTTG
GAAGAAAAAAACATAAAGGAGTTCCTGCAAAGTTTCGTGCACATTGTGCAGATGTTCATTAATA
CCTCTAGCGGCGGAGGATCAGGTGGCGGTGGAAGCGGAGGTGGAGGCTCCGGTGGAGGAGGTAG
TGGCGGAGGTTCTCTTCAAATAACTTGTCCTCCACCGATGTCCGTAGAACATGCGGATATTTGG
GTAAAATCCTATAGCTTGTACAGCCGAGAGCGGTATATCTGCAACAGCGGCTTCAAGCGGAAGG
CCGGCACAAGCAGCCTGACCGAGTGCGTGCTGAACAAGGCCACCAACGTGGCCCACTGGACCAC
CCCTAGCCTGAAGTGCATCAGAGATCCCGCCCTGGTGCATCAGCGGCCTGCCCCTCCAAGCACA
GTGACAACAGCTGGCGTGACCCCCCAGCCTGAGAGCCTGAGCCCTTCTGGAAAAGAGCCTGCCG
CCAGCAGCCCCAGCAGCAACAATACTGCCGCCACCACAGCCGCCATCGTGCCTGGATCTCAGCT
GATGCCCAGCAAGAGCCCTAGCACCGGCACCACCGAGATCAGCAGCCACGAGTCTAGCCACGGC
ACCCCATCTCAGACCACCGCCAAGAACTGGGAGCTGACAGCCAGCGCCTCTCACCAGCCTCCAG
GCGTGTACCCTCAGGGCCACAGCGATACCACAGTGGCCATCAGCACCTCCACCGTGCTGCTGTG
TGGACTGAGCGCCGTGTCACTGCTGGCCTGCTACCTGAAGTCCAGACAGACCCCTCCACTGGCC
AGCGTGGAAATGGAAGCCATGGAAGCACTGCCCGTGACCTGGGGCACCAGCTCCAGAGATGAGG
ATCTGGAAAACTGCTCCCACCACCTG
exemplary multi cistronic CD16, mbIL-15 cargo sequence
SEQ ID NO: 174
ATGTGGCAGCTGTTGCTGCCGACAGCCCTCCTGTTGCTGGTCTCCGCTGGCATGAGAACCGAGG
ATCTGCCTAAGGCCGTGGTGTTCCTGGAACCTCAGTGGTACAGAGTGCTGGAAAAGGACAGCGT
GACCCTGAAGTGCCAGGGCGCCTATTCTCCCGAGGACAATAGCACCCAGTGGTTCCACAACGAG
AGCCTGATCAGCAGCCAGGCCAGCAGCTACTTTATCGATGCCGCCACCGTGGACGACAGCGGCG
AGTACAGATGCCAGACCAATCTGAGCACCCTGAGCGACCCTGTGCAGCTGGAAGTGCACATTGG
ATGGTTGCTGCTGCAAGCCCCTAGATGGGTGTTCAAAGAAGAGGACCCCATCCACCTGAGATGC
CACTCTTGGAAGAACACAGCCCTGCACAAAGTGACCTACCTGCAGAACGGCAAGGGCAGAAAGT
ACTTCCACCACAACAGCGACTTCTACATCCCCAAGGCCACACTGAAGGACTCCGGCTCCTACTT
CTGCAGAGGCCTGGTCGGCAGCAAGAACGTGTCCAGCGAGACAGTGAACATCACCATCACACAG
GGCCTCGCCGTGTCTACCATCAGCAGCTTTTTCCCACCTGGCTATCAGGTGTCCTTCTGCCTGG
TCATGGTGCTGCTGTTCGCCGTGGATACCGGCCTGTACTTCAGCGTCAAGACCAACATCCGGTC
CAGCACCAGAGACTGGAAGGACCACAAGTTCAAGTGGCGGAAGGACCCTCAGGACAAGGGAAGC
GGAGCCACAAACTTCTCTCTGCTGAAGCAGGCAGGAGATGTTGAAGAAAACCCTGGACCTATGG
ATTGGACCTGGATCCTGTTTCTGGTGGCCGCTGCCACAAGAGTGCACAGCAATTGGGTCAACGT
GATCAGCGACCTGAAGAAGATCGAGGACCTGATCCAGAGCATGCACATCGACGCCACACTGTAC
ACCGAGTCCGATGTGCACCCTAGCTGCAAAGTGACCGCCATGAAGTGCTTTCTGCTGGAACTGC
AAGTGATCAGCCTGGAAAGCGGCGACGCCAGCATCCACGATACCGTGGAAAACCTGATCATCCT
GGCCAACAACAGCCTGAGCAGCAACGGCAATGTGACCGAGAGCGGCTGCAAAGAGTGCGAGGAA
CTGGAAGAGAAGAACATCAAAGAGTTCCTCCAGAGCTTCGTCCACATCGTGCAGATGTTCATCA
ACACCAGCTCTGGCGGAGGAAGCGGAGGCGGAGGATCTGGTGGTGGTGGATCTGGCGGCGGTGG
TAGTGGCGGAGGTTCTCTGCAAATCACCTGTCCTCCACCTATGAGCGTGGAACACGCCGACATC
TGGGTCAAGAGCTACAGCCTGTACAGCAGAGAGCGGTACATCTGCAACAGCGGCTTCAAGAGAA
AGGCCGGCACAAGCAGCCTGACCGAGTGTGTGCTGAACAAGGCCACAAACGTGGCCCACTGGAC
CACACCTAGCCTGAAGTGCATCAGAGATCCCGCTCTGGTTCATCAGAGGCCTGCCCCTCCATCT
ACAGTGACAACAGCTGGCGTGACCCCTCAGCCTGAGTCTCTGTCTCCATCTGGAAAAGAGCCTG
CCGCCAGCTCTCCCAGCTCTAACAATACTGCTGCCACCACAGCCGCTATCGTGCCTGGATCTCA
GCTGATGCCTAGCAAGAGCCCTAGCACCGGCACAACAGAGATCAGCTCTCACGAGAGCAGCCAC
GGAACACCTTCTCAGACCACCGCCAAGAATTGGGAGCTGACAGCCTCTGCCTCTCATCAGCCAC
CTGGCGTGTACCCACAGGGCCACTCTGATACAACAGTGGCCATCAGCACCAGCACCGTTCTGCT
GTGTGGCCTGTCTGCTGTTAGCCTGCTGGCCTGCTACCTGAAGTCTAGACAGACACCTCCTCTG
GCCAGCGTGGAAATGGAAGCCATGGAAGCTCTGCCTGTCACATGGGGCACCAGCAGCAGAGATG
AGGAGCTCGAGAATTGGAGCCACCACCTG
exemplary CD19 CAR cargo sequence
SEQ ID NO: 175
ATGCTTCTCCTGGTGACAAGCCTTCTGCTCTGTGAGTTACCACACCCAGCATTCCTCCTGATCC
CAGACATCCAGATGACACAGACTAGATCCTCCCTGTCTGCCTCTCTGGGAGACAGAGTCACCAT
CAGTTGCAGGGCAAGTCAGGACATTAGTAAATATTTAAATTGGTATCAGCAGAAACCAGATGGA
ACTGTTAAACTCCTGATCTACCATACATCAAGATTAGACTCAGGAGTCCCATCAAGGTTCAGTG
GCAGTGGGTCTGGAACAGATTATTCTCTCACCATTAGCAACCTGGAGCAAGAAGATATTGCCAC
TTACTTTTGCCAACAGGGTAATACGCTTCCGTACACGTTCGGAGGGGGGACTAAGTTGGAAATA
ACAGGCTCCACCTCTGGATCCGGCAAGCCCGGATCTGGCGAGGGATCCACCAAGGGCGAGGTGA
AACTGCAGGAGTCAGGACCTGGCCTGGTGGCGCCCTCACAGAGCCTGTCCGTCACATGCACTGT
CTCAGGGGTCTCATTACCCGACTATGGTGTAAGCTGGATTCGCCAGCCTCCACGAAAGGGTCTG
GAGTGGCTGGGAGTAATATGGGGTAGTGAAACCACATACTATAATTCAGCTCTCAAATCCAGAC
TGAGCATCATCAAGGACAACTCCAAGAGCCAAGTTTTCTTAAAAATGAACAGTCTGCAAACTGA
TGACACAGCCATTTACTACTGTGCCAAACATTATTACTACGGTGGTAGCTATGCTATGGACTAC
TGGGGTCAAGGAACCTCAGTCACCGTCTCCTCAGCGGCCGCAATTGAAGTTATGTATCCTCCTC
CTTACCTAGACAATGAGAAGAGCAATGGAACCATTATCCATGTGAAAGGGAAACACCTTTGTCC
AAGTCCCCTATTTCCCGGACCTTCTAAGCCCTTTTGGGTGCTGGTGGTGGTTGGGGGAGTCCTG
GCTTGCTATAGCTTGCTAGTAACAGTGGCCTTTATTATTTTCTGGGTGAGGAGTAAGAGGAGCA
GGCTCCTGCACAGTGACTACATGAACATGACTCCCCGCCGCCCCGGGCCCACCCGCAAGCATTA
CCAGCCCTATGCCCCACCACGCGACTTCGCAGCCTATCGCTCCAGAGTGAAGTTCAGCAGGAGC
GCAGACGCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAAAGCCGAG
AAGGAAGAACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTTACCAGGGTC
TCAGTACAGCCACCAAGGACACCTACGACGCCCTTCACATGCAGGCCCTGCCCCCTCGCTAA
exemplary EGFR CAR cargo sequence
SEQ ID NO: 176
ATGGCACTCCCCGTCACCGCCCTTCTCTTGCCCCTCGCCCTGCTGCTGCATGCTGCCAGGCCCA
TGGACGAAGTGCAGCTCGTGGAGTCCGGTGGAGGACTCGTCCAACCGGGCGGATCCCTTCGCTT
GTCCTGCGCCGCATCAGGCTTCAGCTTCACCAACTATGGCGTCCACTGGGTCAGACAGGCCCCC
GGAAAGGGACTGGAATGGGTGTCCGTGATCTGGAGCGGCGGGAACACCGACTACAACACCTCCG
TGAAGGGCCGGTTCACTATTAGCCGCGACAACTCCAAGAACACTCTGTACCTCCAAATGAACTC
CCTGAGGGCCGAAGATACTGCTGTGTACTATTGCGCGAGAGCCCTGACCTACTACGACTACGAG
TTCGCGTACTGGGGCCAGGGGACTCTCGTGACCGTGTCCAGCGGTGGTGGAGGTTCCGGAGGCG
GAGGTTCTGGTGGCGGGGGATCAGAAATCGTGCTGACTCAGTCCCCTGCGACCTTGTCCCTGAG
CCCTGGAGAACGGGCCACCCTGAGCTGTAGAGCCAGCCAGAGCATCGGGACAAATATTCACTGG
TACCAGCAGAAACCCGGACAAGCACCACGGCTGCTGATCTACTACGCCTCCGAGTCGATTTCCG
GAATCCCGGCTCGCTTTTCGGGGTCTGGATCGGGAACGGACTTCACTCTGACCATCTCGTCGCT
GGAACCCGAGGATTTCGCCGTGTACTACTGCCAACAGAACAACAATTGGCCGACCACGTTCGGC
CAGGGCACCAAGCTCGAGATTAAGGGATCACTGGAAGCGGCCGCAACCACAACACCTGCTCCAA
GGCCCCCCACACCCGCTCCAACTATAGCCAGCCAACCATTGAGCCTCAGACCTGAAGCTTGCAG
GCCCGCAGCAGGAGGCGCCGTCCATACGCGAGGCCTGGACTTCGCGTGTGATATTTATATTTGG
GCCCCTTTGGCCGGAACATGTGGGGTGTTGCTTCTCTCCCTTGTGATCACTCTGTATTGTAAGC
GCGGGAGAAAGAAGCTCCTGTACATCTTCAAGCAGCCTTTTATGCGACCTGTGCAAACCACTCA
GGAAGAAGATGGGTGTTCATGCCGCTTCCCCGAGGAGGAAGAAGGAGGGTGTGAACTGAGGGTG
AAATTTTCTAGAAGCGCCGATGCTCCCGCATATCAGCAGGGTCAGAATCAGCTCTACAATGAAT
TGAATCTCGGCAGGCGAGAAGAGTACGATGTTCTGGACAAGAGACGGGGCAGGGATCCCGAGAT
GGGGGGAAAGCCCCGGAGAAAAAATCCTCAGGAGGGGTTGTACAATGAGCTGCAGAAGGACAAG
ATGGCTGAAGCCTATAGCGAGATCGGAATGAAAGGCGAAAGACGCAGAGGCAAGGGGCATGACG
GTCTGTACCAGGGTCTCTCTACAGCCACCAAGGACACTTATGATGCGTTGCATATGCAAGCCTT
GCCACCCCGCTAA
exemplary GFP cargo sequence
SEQ ID NO: 177
ATGGTGAGCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCA
AGTCCGCCATGCCCGAAGGCTACGTCCAGGAGCGCACCATCTTCTTCAAGGACGACGGCAACTA
CAAGACCCGCGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGATCCGCCACAA
CATCGAGGACGGCAGCGTGCAGCTCGCCGACCACTACCAGCAGAACACCCCCATCGGCGACGGC
CCCGTGCTGCTGCCCGACAACCACTACCTGAGCACCCAGTCCGCCCTGAGCAAAGACCCCAACG
AGAAGCGCGATCACATGGTCCTGCTGGAGTTCGTGACCGCCGCCGGGATCACTCTCGGCATGGA
CGAGCTGTACAAGTGA
exemplary CXCR1 cargo sequence
SEQ ID NO: 178
ATGTCAAATATTACAGATCCACAGATGTGGGATTTTGATGATCTAAATTTCACTGGCATGCCAC
CTGCAGATGAAGATTAGAGCCCCTGTATGCTAGAAACTGAGACACTCAACAAGTATGTTGTGAT
CATCGCCTATGCCCTAGTGTTCCTGCTGAGCCTGCTGGGAAACTCCCTGGTGATGCTGGTCATC
TTATACAGCAGGGTCGGCCGCTCCGTCACTGATGTCTACCTGCTGAACCTGGCCTTGGCCGACC
TACTCTTTGCCCTGACCTTGCCCATCTGGGCCGCCTCCAAGGTGAATGGCTGGATTTTTGGCAC
ATTCCTGTGCAAGGTGGTCTCACTCCTGAAGGAAGTCAACTTCTACAGTGGCATCCTGCTGTTG
GCCTGCATCAGTGTGGACCGTTACCTGGCCATTGTCCATGCCACACGCACACTGACCCAGAAGC
GTCACTTGGTCAAGTTTGTTTGTCTTGGCTGCTGGGGACTGTCTATGAATCTGTCCCTGCCCTT
CTTCCTTTTCCGCCAGGCTTACCATCCAAACAATTCCAGTCCAGTTTGCTATGAGGTCCTGGGA
AATGACACAGCAAAATGGCGGATGGTGTTGCGGATCCTGCCTCACACCTTTGGCTTCATCGTGC
CGCTGTTTGTCATGCTGTTCTGCTATGGATTCACCCTGCGTACACTGTTTAAGGCCCACATGGG
GCAGAAGCACCGAGCCATGAGGGTCATCTTTGCTGTCGTCCTCATCTTCCTGCTTTGCTGGCTG
CCCTACAACCTGGTCCTGCTGGCAGACACCCTCATGAGGACCCAGGTGATCCAGGAGAGCTGTG
AGCGCCGCAACAACATCGGCCGGGCCCTGGATGCCACTGAGATTCTGGGATTTCTCCATAGCTG
CCTCAACCCCATCATCTACGCCTTCATCGGCCAAAATTTTCGCCATGGATTCCTCAAGATCCTG
GCTATGCATGGCCTGGTCAGCAAGGAGTTCTTGGCACGTCATCGTGTTACCTCCTACACTTCTT
CGTCTGTCAATGTCTCTTCCAACCTCTGA
exemplary CXCR3B cargo sequence
SEQ ID NO: 179
ATGGAGTTGAGGAAGTACGGCCCTGGAAGACTGGCGGGGACAGTTATAGGAGGAGCTGCTCAGA
GTAAATCACAGACTAAATCAGACTCAATCACAAAAGAGTTCCTGCCAGGCCTTTAGACAGCCCC
TTCCTCCCCGTTCCCGCCCTCACAGGTGAGTGACCACCAAGTGCTAAATGACGCCGAGGTTGCC
GCCCTCCTGGAGAACTTCAGCTCTTCCTATGACTATGGAGAAAACGAGAGTGACTCGTGCTGTA
CCTCCCCGCCCTGCCCACAGGACTTCAGCCTGAACTTCGACCGGGCCTTCCTGCCAGCCCTCTA
CAGCCTCCTCTTTCTGCTGGGGCTGCTGGGCAACGGCGCGGTGGCAGCCGTGCTGCTGAGCCGG
CGGACAGCCCTGAGCAGCACCGACACCTTCCTGCTCCACCTAGCTGTAGCAGACACGCTGCTGG
TGCTGACACTGCCGCTCTGGGCAGTGGACGCTGCCGTCCAGTGGGTCTTTGGCTCTGGCCTCTG
CAAAGTGGCAGGTGCCCTCTTCAACATCAACTTCTACGCAGGAGCCCTCCTGCTGGCCTGCATC
AGCTTTGACCGCTACCTGAACATAGTTCATGCCACCCAGCTCTACCGCCGGGGGCCCCCGGCCC
GCGTGACCCTCACCTGCCTGGCTGTCTGGGGGCTCTGCCTGCTTTTCGCCCTCCCAGACTTCAT
CTTCCTGTCGGCCCACCACGACGAGCGCCTCAACGCCACCCACTGCCAATACAACTTCCCACAG
GTGGGCCGCACGGCTCTGCGGGTGCTGCAGCTGGTGGCTGGCTTTCTGCTGCCCCTGCTGGTCA
TGGCCTACTGCTATGCCCACATCCTGGCCGTGCTGCTGGTTTCCAGGGGCCAGCGGCGCCTGCG
GGCCATGCGGCTGGTGGTGGTGGTCGTGGTGGCCTTTGCCCTCTGCTGGACCCCCTATCACCTG
GTGGTGCTGGTGGACATCCTCATGGACCTGGGCGCTTTGGCCCGCAACTGTGGCCGAGAAAGCA
GGGTAGACGTGGCCAAGTCGGTCACCTCAGGCCTGGGCTACATGCACTGCTGCCTCAACCCGCT
GCTCTATGCCTTTGTAGGGGTCAAGTTCCGGGAGCGGATGTGGATGCTGCTCTTGCGCCTGGGC
TGCCCCAACCAGAGAGGGCTCCAGAGGCAGCCATCGTCTTCCCGCCGGGATTCATCCTGGTCTG
AGACCTCAGAGGCCTCCTACTCGGGCTTGTGA
exemplary CXCR3 A cargo sequence
SEQ ID NO: 180
ATGGTCCTTGAGGTGAGTGACCACCAAGTGCTAAATGACGCCGAGGTTGCCGCCCTCCTGGAGA
ACTTCAGCTCTTCCTATGACTATGGAGAAAACGAGAGTGACTCGTGCTGTACCTCCCCGCCCTG
CCCACAGGACTTCAGCCTGAACTTCGACCGGGCCTTCCTGCCAGCCCTCTACAGCCTCCTCTTT
CTGCTGGGGCTGCTGGGCAACGGCGCGGTGGCAGCCGTGCTGCTGAGCCGGCGGACAGCCCTGA
GCAGCACCGACACCTTCCTGCTCCACCTAGCTGTAGCAGACACGCTGCTGGTGCTGACACTGCC
GCTCTGGGCAGTGGACGCTGCCGTCCAGTGGGTCTTTGGCTCTGGCCTCTGCAAAGTGGCAGGT
GCCCTCTTCAACATCAACTTCTACGCAGGAGCCCTCCTGCTGGCCTGCATCAGCTTTGACCGCT
ACCTGAACATAGTTCATGCCACCCAGCTCTACCGCCGGGGGCCCCCGGCCCGCGTGACCCTCAC
CTGCCTGGCTGTCTGGGGGCTCTGCCTGCTTTTCGCCCTCCCAGACTTCATCTTCCTGTCGGCC
CACCACGACGAGCGCCTCAACGCCACCCACTGCCAATACAACTTCCCACAGGTGGGCCGCACGG
CTCTGCGGGTGCTGCAGCTGGTGGCTGGCTTTCTGCTGCCCCTGCTGGTCATGGCCTACTGCTA
TGCCCACATCCTGGCCGTGCTGCTGGTTTCCAGGGGCCAGCGGCGCCTGCGGGCCATGCGGCTG
GTGGTGGTGGTCGTGGTGGCCTTTGCCCTCTGCTGGACCCCCTATCACCTGGTGGTGCTGGTGG
ACATCCTCATGGACCTGGGCGCTTTGGCCCGCAACTGTGGCCGAGAAAGCAGGGTAGACGTGGC
CAAGTCGGTCACCTCAGGCCTGGGCTACATGCACTGCTGCCTCAACCCGCTGCTCTATGCCTTT
GTAGGGGTCAAGTTCCGGGAGCGGATGTGGATGCTGCTCTTGCGCCTGGGCTGCCCCAACCAGA
GAGGGCTCCAGAGGCAGCCATCGTCTTCCCGCCGGGATTCATCCTGGTCTGAGACCTCAGAGGC
CTCCTACTCGGGCTTGTGA
exemplary CCR5 cargo sequence
SEQ ID NO: 181
ATGGATTATCAAGTGTCAAGTCCAATCTATGAGATCAATTATTATACATCGGAGCCCTGCCAAA
AAATCAATGTGAAGCAAATCGCAGCCCGCCTCCTGCCTCCGCTCTACTCACTGGTGTTCATCTT
TGGTTTTGTGGGCAACATGCTGGTCATCCTCATCCTGATAAACTGCAAAAGGCTGAAGAGCATG
ACTGACATCTACCTGCTCAACCTGGCCATCTCTGACCTGTTTTTCCTTCTTACTGTCCCCTTCT
GGGCTCACTATGCTGCCGCCCAGTGGGACTTTGGAAATACAATGTGTCAACTCTTGACAGGGCT
CTATTTTATAGGCTTCTTCTCTGGAATCTTCTTCATCATCCTCCTGACAATCGATAGGTACCTG
GCTGTCGTCCATGCTGTGTTTGCTTTAAAAGCCAGGACGGTCACCTTTGGGGTGGTGACAAGTG
TGATCACTTGGGTGGTGGCTGTGTTTGCGTCTCTCCCAGGAATCATCTTTACCAGATCTCAAAA
AGAAGGTCTTCATTAGACCTGCAGCTCTCATTTTCCATACAGTCAGTATCAATTCTGGAAGAAT
TTCCAGACATTAAAGATAGTCATCTTGGGGCTGGTCCTGCCGCTGCTTGTCATGGTCATCTGCT
ACTCGGGAATCCTAAAAACTCTGCTTCGGTGTCGAAATGAGAAGAAGAGGCACAGGGCTGTGAG
GCTTATCTTCACCATCATGATTGTTTATTTTCTCTTCTGGGCTCCCTACAACATTGTCCTTCTC
CTGAACACCTTCCAGGAATTCTTTGGCCTGAATAATTGCAGTAGCTCTAACAGGTTGGACCAAG
CTATGCAGGTGACAGAGACTCTTGGGATGACGCACTGCTGCATCAACCCCATCATCTATGCCTT
TGTCGGGGAGAAGTTCAGAAACTACCTCTTAGTCTTCTTCCAAAAGCACATTGCCAAACGCTTC
TGCAAATGCTGTTCTATTTTCCAGCAAGAGGCTCCCGAGCGAGCAAGCTCAGTTTACACCCGAT
CCACTGGGGAGCAGGAAATATCTGTGGGCTTGTGA
exemplary CCR2 cargo sequence
SEQ ID NO: 182
ATGCTGTCCACATCTCGTTCTCGGTTTATCAGAAATACCAACGAGAGCGGTGAAGAAGTCACCA
CCTTTTTTGATTATGATTACGGTGCTCCCTGTCATAAATTTGACGTGAAGCAAATTGGGGCCCA
ACTCCTGCCTCCGCTCTACTCGCTGGTGTTCATCTTTGGTTTTGTGGGCAACATGCTGGTCGTC
CTCATCTTAATAAACTGCAAAAAGCTGAAGTGCTTGAGTGACATTTACCTGCTCAACCTGGCCA
TCTCTGATCTGCTTTTTCTTATTACTCTCCCATTGTGGGCTCACTCTGCTGCAAATGAGTGGGT
CTTTGGGAATGCAATGTGCAAATTATTCACAGGGCTGTATCACATCGGTTATTTTGGCGGAATC
TTCTTCATCATCCTCCTGACAATCGATAGATACCTGGCTATTGTCCATGCTGTGTTTGCTTTAA
AAGCCAGGACGGTCACCTTTGGGGTGGTGACAAGTGTGATCACCTGGTTGGTGGCTGTGTTTGC
TTCTGTCCCAGGAATCATCTTTACTAAATGCCAGAAAGAAGATTCTGTTTATGTCTGTGGCCCT
TATTTTCCACGAGGATGGAATAATTTCCACACAATAATGAGGAACATTTTGGGGCTGGTCCTGC
CGCTGCTCATCATGGTCATCTGCTACTCGGGAATCCTGAAAACCCTGCTTCGGTGTCGAAACGA
GAAGAAGAGGCATAGGGCAGTGAGAGTCATCTTCACCATCATGATTGTTTACTTTCTCTTCTGG
ACTCCCTATAATATTGTCATTCTCCTGAACACCTTCCAGGAATTCTTCGGCCTGAGTAACTGTG
AAAGCACCAGTCAACTGGACCAAGCCACGCAGGTGACAGAGACTCTTGGGATGACTCACTGCTG
CATCAATCCCATCATCTATGCCTTCGTTGGGGAGAAGTTCAGAAGCCTTTTTCACATAGCTCTT
GGCTGTAGGATTGCCCCACTCCAAAAACCAGTGTGTGGAGGTCCAGGAGTGAGACCAGGAAAGA
ATGTGAAAGTGACTACACAAGGACTCCTCGATGGTCGTGGAAAAGGAAAGTCAATTGGCAGAGC
CCCTGAAGCCAGTCTTCAGGACAAAGAAGGAGCCTAG

In some embodiments, a gene product of interest comprises or consists of an amino acid sequence of any one of SEQ ID NOs: 161, 164, or 183-200. In some embodiments, a gene product of interest comprises or consists of an amino acid sequence that is at least 85%, 90%, 95%, 98% or 99% identical to any one of SEQ ID NOs: 161, 164, or 183-200.

exemplary linker amino acid sequence
SEQ ID NO: 183
SGGGSGGGGSGGGGSGGGGSGGGSLQ
exemplary CD16 amino acid sequence
SEQ ID NO: 184
MWQLLLPTALLLLVSAGMRTEDLPKAVVFLEPQWYRVLEKDSVTLKCQGAYSPEDNSTQWFHNE
SLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQ
GLAVSTISSFFPPGYQVSFCLVMVLLFAVDTGLYFSVKTNIRSSTRDWKDHKFKWRKDPQDK
exemplary CD47 amino acid sequence
SEQ ID NO: 185
MWPLVAALLLGSACCGSAQLLFNKTKSVEFTFCNDTVVIPCFVTNMEAQNTTEVYVKWKFKGRD
IYTFDGALNKSTVPTDFSSAKIEVSQLLKGDASLKMDKSDAVSHTGNYTCEVTELTREGETIIE
LKYRVVSWFSPNENILIVIFPIFAILLFWGQFGIKTLKYRSGGMDEKTIALLVAGLVITVIVIV
GAILFVPGEYSLKNATGLGLIVTSTGILILLHYYVFSTAIGLTSFVIAILVIQVIAYILAVVGL
SLCIAACIPMHGPLLISGLSILALAQLLGLVYMKFVASNQKTIQPPRKAVEEPLNAFKESKGMM
NDE
exemplary IL15 amino acid sequence
SEQ ID NO: 186
NWVNVISDLKKIEDLIQSMHIDATLYTESDVHPSCKVTAMKCFLLELQVISLESGDASIHDTVE
NLIILANNSLSSNGNVTESGCKECEELEEKNIKEFLQSFVHIVQMFINTS
exemplary IgE-IL15 amino acid sequence
SEQ ID NO: 187
MDWTWILFLVAAATRVHSNWVNVISDLKKIEDLIQSMHIDATLYTESDVHPSCKVTAMKCFLLE
LQVISLESGDASIHDTVENLIILANNSLSSNGNVTESGCKECEELEEKNIKEFLQSFVHIVQMF
INTS
exemplary IgE-IL15 pro-peptide amino acid sequence
SEQ ID NO: 188
MDWTWILFLVAAATRVHSGIHVFILGCFSAGLPKTEANWVNVISDLKKIEDLIQSMHIDATLYT
ESDVHPSCKVTAMKCFLLELQVISLESGDASIHDTVENLIILANNSLSSNGNVTESGCKECEEL
EEKNIKEFLQSFVHIVQMFINTS
exemplary IL15Rα amino acid sequence
SEQ ID NO: 189
ITCPPPMSVEHADIWVKSYSLYSRERYICNSGFKRKAGTSSLTECVLNKATNVAHWTTPSLKCI
RDPALVHQRPAPPSTVTTAGVTPQPESLSPSGKEPAASSPSSNNTAATTAAIVPGSQLMPSKSP
STGTTEISSHESSHGTPSQTTAKNWELTASASHQPPGVYPQGHSDTTVAISTSTVLLCGLSAVS
LLACYLKSRQTPPLASVEMEAMEALPVTWGTSSRDEDLENCSHHL
exemplary mbIL-15 amino acid sequence
SEQ ID NO: 190
MDWTWILFLVAAATRVHSNWVNVISDLKKIEDLIQSMHIDATLYTESDVHPSCKVTAMKCFLLE
LQVISLESGDASIHDTVENLIILANNSLSSNGNVTESGCKECEELEEKNIKEFLQSFVHIVQMF
INTSSGGGSGGGGSGGGGSGGGGSGGGSLQITCPPPMSVEHADIWVKSYSLYSRERYICNSGFK
RKAGTSSLTECVLNKATNVAHWTTPSLKCIRDPALVHQRPAPPSTVTTAGVTPQPESLSPSGKE
PAASSPSSNNTAATTAAIVPGSQLMPSKSPSTGTTEISSHESSHGTPSQTTAKNWELTASASHQ
PPGVYPQGHSDTTVAISTSTVLLCGLSAVSLLACYLKSRQTPPLASVEMEAMEALPVTWGTSSR
DEDLENCSHHL
exemplary mbIL-15 amino acid sequence
SEQ ID NO: 191
MDWTWILFLVAAATRVHSGIHVFILGCFSAGLPKTEANWVNVISDLKKIEDLIQSMHIDATLYT
ESDVHPSCKVTAMKCFLLELQVISLESGDASIHDTVENLIILANNSLSSNGNVTESGCKECEEL
EEKNIKEFLQSFVHIVQMFINTSSGGGSGGGGSGGGGSGGGGSGGGSLQITCPPPMSVEHADIW
VKSYSLYSRERYICNSGFKRKAGTSSLTECVLNKATNVAHWTTPSLKCIRDPALVHQRPAPPST
VTTAGVTPQPESLSPSGKEPAASSPSSNNTAATTAAIVPGSQLMPSKSPSTGTTEISSHESSHG
TPSQTTAKNWELTASASHQPPGVYPQGHSDTTVAISTSTVLLCGLSAVSLLACYLKSRQTPPLA
SVEMEAMEALPVTWGTSSRDEDLENCSHHL
exemplary multi cistronic CD16, mbIL-15 amino acid sequence
SEQ ID NO: 192
MWQLLLPTALLLLVSAGMRTEDLPKAVVFLEPQWYRVLEKDSVTLKCQGAYSPEDNSTQWFHNE
SLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQ
GLAVSTISSFFPPGYQVSFCLVMVLLFAVDTGLYFSVKTNIRSSTRDWKDHKFKWRKDPQDKGS
GATNFSLLKQAGDVEENPGPMDWTWILFLVAAATRVHSNWVNVISDLKKIEDLIQSMHIDATLY
TESDVHPSCKVTAMKCFLLELQVISLESGDASIHDTVENLIILANNSLSSNGNVTESGCKECEE
LEEKNIKEFLQSFVHIVQMFINTSSGGGSGGGGSGGGGSGGGGSGGGSLQITCPPPMSVEHADI
WVKSYSLYSRERYICNSGFKRKAGTSSLTECVLNKATNVAHWTTPSLKCIRDPALVHQRPAPPS
TVTTAGVTPQPESLSPSGKEPAASSPSSNNTAATTAAIVPGSQLMPSKSPSTGTTEISSHESSH
GTPSQTTAKNWELTASASHQPPGVYPQGHSDTTVAISTSTVLLCGLSAVSLLACYLKSRQTPPL
ASVEMEAMEALPVTWGTSSRDEDLENCSHHL
exemplary CD19 CAR amino acid sequence
SEQ ID NO: 193
MLLLVTSLLLCELPHPAFLLIPDIQMTQTTSSLSASLGDRVTISCRASQDISKYLNWYQQKPDG
TVKLLIYHTSRLHSGVPSRFSGSGSGTDYSLTISNLEQEDIATYFCQQGNTLPYTFGGGTKLEI
TGSTSGSGKPGSGEGSTKGEVKLQESGPGLVAPSQSLSVTCTVSGVSLPDYGVSWIRQPPRKGL
EWLGVIWGSETTYYNSALKSRLTIIKDNSKSQVFLKMNSLQTDDTAIYYCAKHYYYGGSYAMDY
WGQGTSVTVSSAAAIEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQEGLYNELQKDKMAEAY
SEIGMKGERRRGKGHDGLYQGLSTATKDTYDALHMQALPPR
exemplary EGFR CAR amino acid sequence
SEQ ID NO: 194
MALPVTALLLPLALLLHAARPMDEVQLVESGGGLVQPGGSLRLSCAASGFSFTNYGVHWVRQAP
GKGLEWVSVIWSGGNTDYNTSVKGRFTISRDNSKNTLYLQMNSLRAEDTAVYYCARALTYYDYE
FAYWGQGTLVTVSSGGGGSGGGGSGGGGSEIVLTQSPATLSLSPGERATLSCRASQSIGTNIHW
YQQKPGQAPRLLIYYASESISGIPARFSGSGSGTDFTLTISSLEPEDFAVYYCQQNNNWPTTFG
QGTKLEIKGSLEAAATTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
exemplary GFP amino acid sequence
SEQ ID NO: 195
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKG
IDFKEDGNILGHKLEYNYNSHNVYIMADKQKNGIKVNFKIRHNIEDGSVQLADHYQQNTPIGDG
PVLLPDNHYLSTQSALSKDPNEKRDHMVLLEFVTAAGITLGMDELYK
exemplary CXCR1 amino acid sequence
SEQ ID NO: 196
MSNITDPQMWDFDDLNFTGMPPADEDYSPCMLETETLNKYVVIIAYALVFLLSLLGNSLVMLVI
LYSRVGRSVTDVYLLNLALADLLFALTLPIWAASKVNGWIFGTFLCKVVSLLKEVNFYSGILLL
ACISVDRYLAIVHATRTLTQKRHLVKFVCLGCWGLSMNLSLPFFLFRQAYHPNNSSPVCYEVLG
NDTAKWRMVLRILPHTFGFIVPLFVMLFCYGFTLRTLFKAHMGQKHRAMRVIFAVVLIFLLCWL
PYNLVLLADTLMRTQVIQESCERRNNIGRALDATEILGFLHSCLNPIIYAFIGQNFRHGFLKIL
AMHGLVSKEFLARHRVTSYTSSSVNVSSNL
exemplary CXCR3B amino acid sequence
SEQ ID NO: 197
MELRKYGPGRLAGTVIGGAAQSKSQTKSDSITKEFLPGLYTAPSSPFPPSQVSDHQVLNDAEVA
ALLENFSSSYDYGENESDSCCTSPPCPQDFSLNFDRAFLPALYSLLFLLGLLGNGAVAAVLLSR
RTALSSTDTFLLHLAVADTLLVLTLPLWAVDAAVQWVFGSGLCKVAGALFNINFYAGALLLACI
SFDRYLNIVHATQLYRRGPPARVTLTCLAVWGLCLLFALPDFIFLSAHHDERLNATHCQYNFPQ
VGRTALRVLQLVAGFLLPLLVMAYCYAHILAVLLVSRGORRLRAMRLVVVVVVAFALCWTPYHL
VVLVDILMDLGALARNCGRESRVDVAKSVTSGLGYMHCCLNPLLYAEVGVKFRERMWMLLLRLG
CPNQRGLQRQPSSSRRDSSWSETSEASYSGL
exemplary CXCR3 A amino acid sequence
SEQ ID NO: 198
MVLEVSDHQVLNDAEVAALLENFSSSYDYGENESDSCCTSPPCPQDFSLNFDRAFLPALYSLLF
LLGLLGNGAVAAVLLSRRTALSSTDTFLLHLAVADTLLVLTLPLWAVDAAVQWVFGSGLCKVAG
ALFNINFYAGALLLACISFDRYLNIVHATQLYRRGPPARVTLTCLAVWGLCLLFALPDFIFLSA
HHDERLNATHCQYNFPQVGRTALRVLQLVAGFLLPLLVMAYCYAHILAVLLVSRGQRRLRAMRL
VVVVVVAFALCWTPYHLVVLVDILMDLGALARNCGRESRVDVAKSVTSGLGYMHCCLNPLLYAF
VGVKFRERMWMLLLRLGCPNQRGLQRQPSSSRRDSSWSETSEASYSGL
exemplary CCR5 amino acid sequence
SEQ ID NO: 199
MDYQVSSPIYDINYYTSEPCQKINVKQIAARLLPPLYSLVFIFGFVGNMLVILILINCKRLKSM
TDIYLLNLAISDLFFLLTVPFWAHYAAAQWDFGNTMCQLLTGLYFIGFFSGIFFIILLTIDRYL
AVVHAVFALKARTVTFGVVTSVITWVVAVFASLPGIIFTRSQKEGLHYTCSSHFPYSQYQFWKN
FQTLKIVILGLVLPLLVMVICYSGILKTLLRCRNEKKRHRAVRLIFTIMIVYFLFWAPYNIVLL
LNTFQEFFGLNNCSSSNRLDQAMQVTETLGMTHCCINPIIYAFVGEKFRNYLLVFFQKHIAKRF
CKCCSIFQQEAPERAS SVYTRSTGEQEISVGL
exemplary CCR2 cargo sequence
SEQ ID NO: 200
MLSTSRSRFIRNTNESGEEVTTFFDYDYGAPCHKFDVKQIGAQLLPPLYSLVFIFGFVGNMLVV
LILINCKKLKCLTDIYLLNLAISDLLFLITLPLWAHSAANEWVFGNAMCKLFTGLYHIGYFGGI
FFIILLTIDRYLAIVHAVFALKARTVTFGVVTSVITWLVAVFASVPGIIFTKCQKEDSVYVCGP
YFPRGWNNFHTIMRNILGLVLPLLIMVICYSGILKTLLRCRNEKKRHRAVRVIFTIMIVYFLFW
TPYNIVILLNTFQEFFGLSNCESTSQLDQATQVTETLGMTHCCINPIIYAFVGEKFRSLFHIAL
GCRIAPLQKPVCGGPGVRPGKNVKVTTQGLLDGRGKGKSIGRAPEASLQDKEGA

AAV Capsids

In some embodiments, the present disclosure provides one or more polynucleotide constructs (e.g., knock-in cassettes) packaged into an AAV capsid. In some embodiments, an AAV capsid is from or derived from an AAV capsid of an AAV2, 3, 4, 5, 6, 7, 8, 9, or 10 serotype, or one or more hybrids thereof. In some embodiments, an AAV capsid is from an AAV ancestral serotype. In some embodiments, an AAV capsid is an ancestral (Anc) AAV capsid. An Anc capsid is created from a construct sequence that is constructed using evolutionary probabilities and evolutionary modeling to determine a probable ancestral sequence. In some embodiments, an AAV capsid has been modified in a manner known in the art (see e.g., BUning and Srivastava, Capsid modifications for targeting and improving the efficacy of AAV vectors, Mol Ther Methods Clin Dev. 2019)

In some embodiments, as provided herein, any combination of AAV capsids and AAV constructs (e.g., comprising AAV ITRs) may be used in recombinant AAV (rAAV) particles of the present disclosure. In some embodiments, an AAV ITR is from or derived from an AAV ITR of AAV2, 3, 4, 5, 6, 7, 8, 9, or 10. For example, wild-type or variant AA6 ITRs and AAV6 capsid, wild-type or variant AAV2 ITRs and AAV6 capsid, etc. In some embodiments of the present disclosure, an AAV particle is wholly comprised of AAV6 components (e.g., capsid and ITRs are AAV6 serotype). In some embodiments, an AAV particle is an AAV6/2, AAV6/8 or AAV6/9 particle (e.g., an AAV2, AAV8 or AAV9 capsid with an AAV construct having AAV6 ITRs).

Exemplary AAV Constructs

In some embodiments, a donor template is included within an AAV construct. In some embodiments, an AAV construct sequence comprises or consists of the sequence of any one of SEQ ID NO: 201-204. In some embodiments, an exemplary AAV construct is represented by SEQ ID NO: 201. In some embodiments, an exemplary AAV construct is represented by SEQ ID NO: 202. In some embodiments, an exemplary AAV construct is represented by SEQ ID NO: 203. In some embodiments, an exemplary AAV construct is represented by SEQ ID NO: 204. In some embodiments, an exemplary AAV construct is at least 80%, 85%, 90%, 95%, 98%, or 99% identical to a sequence represented by SEQ ID NO: 201-204.

exemplary AAV construct for donor template insertion
at GAPDH locus
SEQ ID NO: 201
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGTCGACGAAGACTGTGGATGGCCCCTCCGGGAAACTGTGGCGTGATGGCCG
CGGGGCTCTCCAGAACATCATCCCTGCCTCTACTGGCGCTGCCAAGGCTGTGGGCAAGGTCATC
CCTGAGCTGAACGGGAAGCTCACTGGCATGGCCTTCCGTGTCCCCACTGCCAACGTGTCAGTGG
TGGACCTGACCTGCCGTCTAGAAAAACCTGCCAAATATGATGACATCAAGAAGGTGGTGAAGCA
GGCGTCGGAGGGCCCCCTCAAGGGCATCCTGGGCTACACTGAGCACCAGGTGGTCTCCTCTGAC
TTCAACAGCGACACCCACTCCTCCACCTTTGACGCTGGGGCTGGCATTGCCCTCAACGACCACT
TTGTCAAGCTCATTTCCTGGTATGTGGCTGGGGCCAGAGACTGGCTCTTAAAAAGTGCAGGGTC
TGGCGCCCTCTGGTGGCTGGCTCAGAAAAAGGGCCCTGACAACTCTTTACATCTTCTAGGTATG
ACAACGAGTTCGGATATAGCAATAGAGTGGTCGATCTGATGGCTCATATGGCTAGCAAAGAGGG
AAGCGGAGCTACTAACTTCAGCCTGCTGAAGCAGGCTGGAGACGTGGAGGAGAACCCTGGACCT
ATGTGGCAACTGCTGCTGCCTACAGCTCTGCTGCTTCTGGTGTCTGCCGGCATGAGAACCGAGG
ATCTGCCTAAGGCCGTGGTGTTCCTGGAACCTCAGTGGTACAGAGTGCTGGAAAAGGACAGCGT
GACCCTGAAGTGCCAGGGCGCCTATTCTCCCGAGGACAATAGCACCCAGTGGTTCCACAACGAG
AGCCTGATCAGCAGCCAGGCCAGCAGCTACTTTATCGATGCCGCCACCGTGGACGACAGCGGCG
AGTACAGATGCCAGACCAATCTGAGCACCCTGAGCGACCCTGTGCAGCTGGAAGTGCACATTGG
ATGGTTGCTGCTGCAAGCCCCTAGATGGGTGTTCAAAGAAGAGGACCCCATCCACCTGAGATGC
CACTCTTGGAAGAACACAGCCCTGCACAAAGTGACCTACCTGCAGAACGGCAAGGGCAGAAAGT
ACTTCCACCACAACAGCGACTTCTACATCCCCAAGGCCACACTGAAGGACTCCGGCTCCTACTT
CTGCAGAGGCCTGGTCGGCAGCAAGAACGTGTCCAGCGAGACAGTGAACATCACCATCACACAG
GGCCTCGCCGTGTCTACCATCAGCAGCTTTTTCCCACCTGGCTATCAGGTGTCCTTCTGCCTGG
TCATGGTGCTGCTGTTCGCCGTGGATACCGGCCTGTACTTCAGCGTCAAGACCAACATCCGGTC
CAGCACCAGAGACTGGAAGGACCACAAGTTCAAGTGGCGGAAGGACCCTCAGGACAAGTAAGCG
GCCGCGTCGAGTCTAGAGGGCCCGTTTAAACCCGCTGATCAGCCTCGACTGTGCCTTCTAGTTG
CCAGCCATCTGTTGTTTGCCCCTCCCCCGTGCCTTCCTTGACCCTGGAAGGTGCCACTCCCACT
GTCCTTTCCTAATAAAATGAGGAAATTGCATCGCATTGTCTGAGTAGGTGTCATTCTATTCTGG
GGGGTGGGGTGGGGCAGGACAGCAAGGGGGAGGATTGGGAAGACAATAGCAGGCATGCTGGGGA
TGCGGTGGGCTCTATGGATTTGGCTACAGCAACAGGGTGGTGGACCTCATGGCCCACATGGCCT
CCAAGGAGTAAGACCCCTGGACCACCAGCCCCAGCAAGAGCACAAGAGGAAGAGAGAGACCCTC
ACTGCTGGGGAGTCCCTGCCACACTCAGTCCCCCACCACACTGAATCTCCCCTCCTCACAGTTG
CCATGTAGACCCCTTGAAGAGGGGAGGGGCCTAGGGAGCCGCACCTTGTCATGTACCATCAATA
AAGTACCCTGTGCTCAACCAGTTACTTGTCCTGTCTTATTCTAGGGTCTGGGGCAGAGGGGAGG
GAAGCTGGGCTTGTGTCAAGGTGAGACATTCTTGCTGGGGAGGGACCTGGTATGTTCTCCTCAG
ACTGAGGGTAGGGCCTCCAAACAGCCTTGCTTGCTTCGAGAACCATTTGCTTCCCGCTCAGACG
TCTTGAGTGCTACAGGAAGCTGGCACCACTACTTCAGAGAACAAGGCCTTTTCCTCTCCTCGCT
CCAGTAGATCTAGGAACCCCTAGTGATGGAGTTGGCCACTCCCTCTCTGCGCGCTCGCTCGCTC
ACTGAGGCCGGGCGACCAAAGGTCGCCCGACGCCCGGGCTTTGCCCGGGCGGCCTCAGTGAGCG
AGCGAGCGCGCAGCTGCCTGCAGG
exemplary AAV construct for donor template insertion
at GAPDH locus
SEQ ID NO: 202
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGTCGACGAAGACTGTGGATGGCCCCTCCGGGAAACTGTGGCGTGATGGCCG
CGGGGCTCTCCAGAACATCATCCCTGCCTCTACTGGCGCTGCCAAGGCTGTGGGCAAGGTCATC
CCTGAGCTGAACGGGAAGCTCACTGGCATGGCCTTCCGTGTCCCCACTGCCAACGTGTCAGTGG
TGGACCTGACCTGCCGTCTAGAAAAACCTGCCAAATATGATGACATCAAGAAGGTGGTGAAGCA
GGCGTCGGAGGGCCCCCTCAAGGGCATCCTGGGCTACACTGAGCACCAGGTGGTCTCCTCTGAC
TTCAACAGCGACACCCACTCCTCCACCTTTGACGCTGGGGCTGGCATTGCCCTCAACGACCACT
TTGTCAAGCTCATTTCCTGGTATGTGGCTGGGGCCAGAGACTGGCTCTTAAAAAGTGCAGGGTC
TGGCGCCCTCTGGTGGCTGGCTCAGAAAAAGGGCCCTGACAACTCTTTACATCTTCTAGGTATG
ACAACGAGTTCGGATATAGCAATAGAGTGGTCGATCTGATGGCTCATATGGCTAGCAAAGAGGG
AAGCGGAGCTACTAACTTCAGCCTGCTGAAGCAGGCTGGAGACGTGGAGGAGAACCCTGGACCT
ATGGTGAGCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCA
AGTCCGCCATGCCCGAAGGCTACGTCCAGGAGCGCACCATCTTCTTCAAGGACGACGGCAACTA
CAAGACCCGCGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGATCCGCCACAA
CATCGAGGACGGCAGCGTGCAGCTCGCCGACCACTACCAGCAGAACACCCCCATCGGCGACGGC
CCCGTGCTGCTGCCCGACAACCACTACCTGAGCACCCAGTCCGCCCTGAGCAAAGACCCCAACG
AGAAGCGCGATCACATGGTCCTGCTGGAGTTCGTGACCGCCGCCGGGATCACTCTCGGCATGGA
CGAGCTGTACAAGTGAGCGGCCGCGTCGAGTCTAGAGGGCCCGTTTAAACCCGCTGATCAGCCT
CGACTGTGCCTTCTAGTTGCCAGCCATCTGTTGTTTGCCCCTCCCCCGTGCCTTCCTTGACCCT
GGAAGGTGCCACTCCCACTGTCCTTTCCTAATAAAATGAGGAAATTGCATCGCATTGTCTGAGT
AGGTGTCATTCTATTCTGGGGGGTGGGGTGGGGCAGGACAGCAAGGGGGAGGATTGGGAAGACA
ATAGCAGGCATGCTGGGGATGCGGTGGGCTCTATGGATTTGGCTACAGCAACAGGGTGGTGGAC
CTCATGGCCCACATGGCCTCCAAGGAGTAAGACCCCTGGACCACCAGCCCCAGCAAGAGCACAA
GAGGAAGAGAGAGACCCTCACTGCTGGGGAGTCCCTGCCACACTCAGTCCCCCACCACACTGAA
TCTCCCCTCCTCACAGTTGCCATGTAGACCCCTTGAAGAGGGGAGGGGCCTAGGGAGCCGCACC
TTGTCATGTACCATCAATAAAGTACCCTGTGCTCAACCAGTTACTTGTCCTGTCTTATTCTAGG
GTCTGGGGCAGAGGGGAGGGAAGCTGGGCTTGTGTCAAGGTGAGACATTCTTGCTGGGGAGGGA
CCTGGTATGTTCTCCTCAGACTGAGGGTAGGGCCTCCAAACAGCCTTGCTTGCTTCGAGAACCA
TTTGCTTCCCGCTCAGACGTCTTGAGTGCTACAGGAAGCTGGCACCACTACTTCAGAGAACAAG
GCCTTTTCCTCTCCTCGCTCCAGTAGATCTAGGAACCCCTAGTGATGGAGTTGGCCACTCCCTC
TCTGCGCGCTCGCTCGCTCACTGAGGCCGGGCGACCAAAGGTCGCCCGACGCCCGGGCTTTGCC
CGGGCGGCCTCAGTGAGCGAGCGAGCGCGCAGCTGCCTGCAGG
exemplary AAV construct for donor template insertion
at GAPDH locus
SEQ ID NO: 203
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGTCGACGAAGACTGTGGATGGCCCCTCCGGGAAACTGTGGCGTGATGGCCG
CGGGGCTCTCCAGAACATCATCCCTGCCTCTACTGGCGCTGCCAAGGCTGTGGGCAAGGTCATC
CCTGAGCTGAACGGGAAGCTCACTGGCATGGCCTTCCGTGTCCCCACTGCCAACGTGTCAGTGG
TGGACCTGACCTGCCGTCTAGAAAAACCTGCCAAATATGATGACATCAAGAAGGTGGTGAAGCA
GGCGTCGGAGGGCCCCCTCAAGGGCATCCTGGGCTACACTGAGCACCAGGTGGTCTCCTCTGAC
TTCAACAGCGACACCCACTCCTCCACCTTTGACGCTGGGGCTGGCATTGCCCTCAACGACCACT
TTGTCAAGCTCATTTCCTGGTATGTGGCTGGGGCCAGAGACTGGCTCTTAAAAAGTGCAGGGTC
TGGCGCCCTCTGGTGGCTGGCTCAGAAAAAGGGCCCTGACAACTCTTTACATCTTCTAGGTATG
ACAACGAGTTCGGATATAGCAATAGAGTGGTCGATCTGATGGCTCATATGGCTAGCAAAGAGGG
AAGCGGAGCTACTAACTTCAGCCTGCTGAAGCAGGCTGGAGACGTGGAGGAGAACCCTGGACCT
ATGCTTCTCCTGGTGACAAGCCTTCTGCTCTGTGAGTTACCACACCCAGCATTCCTCCTGATCC
CAGACATCCAGATGACACAGACTAGATCCTCCCTGTCTGCCTCTCTGGGAGACAGAGTCACCAT
CAGTTGCAGGGCAAGTCAGGACATTAGTAAATATTTAAATTGGTATCAGCAGAAACCAGATGGA
ACTGTTAAACTCCTGATCTACCATACATCAAGATTAGACTCAGGAGTCCCATCAAGGTTCAGTG
GCAGTGGGTCTGGAACAGATTATTCTCTCACCATTAGCAACCTGGAGCAAGAAGATATTGCCAC
TTACTTTTGCCAACAGGGTAATACGCTTCCGTACACGTTCGGAGGGGGGACTAAGTTGGAAATA
ACAGGCTCCACCTCTGGATCCGGCAAGCCCGGATCTGGCGAGGGATCCACCAAGGGCGAGGTGA
AACTGCAGGAGTCAGGACCTGGCCTGGTGGCGCCCTCACAGAGCCTGTCCGTCACATGCACTGT
CTCAGGGGTCTCATTACCCGACTATGGTGTAAGCTGGATTCGCCAGCCTCCACGAAAGGGTCTG
GAGTGGCTGGGAGTAATATGGGGTAGTGAAACCACATACTATAATTCAGCTCTCAAATCCAGAC
TGACCATCATCAAGGACAACTCCAAGAGCCAAGTTTTCTTAAAAATGAACAGTCTGCAAACTGA
TGACACAGCCATTTACTACTGTGCCAAACATTATTACTACGGTGGTAGCTATGCTATGGACTAC
TGGGGTCAAGGAACCTCAGTCACCGTCTCCTCAGCGGCCGCAATTGAAGTTATGTATCCTCCTC
CTTACCTAGACAATGAGAAGAGCAATGGAACCATTATCCATGTGAAAGGGAAACACCTTTGTCC
AAGTCCCCTATTTCCCGGACCTTCTAAGCCCTTTTGGGTGCTGGTGGTGGTTGGGGGAGTCCTG
GCTTGCTATAGCTTGCTAGTAACAGTGGCCTTTATTATTTTCTGGGTGAGGAGTAAGAGGAGCA
GGCTCCTGCACAGTGACTACATGAACATGACTCCCCGCCGCCCCGGGCCCACCCGCAAGCATTA
CCAGCCCTATGCCCCACCACGCGACTTCGCAGCCTATCGCTCCAGAGTGAAGTTCAGCAGGAGC
GCAGACGCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAAAGCCGAG
AAGGAAGAACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTTACCAGGGTC
TCAGTACAGCCACCAAGGACACCTACGACGCCCTTCACATGCAGGCCCTGCCCCCTCGCTAAAG
CGGCCGCGTCGAGTCTAGAGGGCCCGTTTAAACCCGCTGATCAGCCTCGACTGTGCCTTCTAGT
TGCCAGCCATCTGTTGTTTGCCCCTCCCCCGTGCCTTCCTTGACCCTGGAAGGTGCCACTCCCA
CTGTCCTTTCCTAATAAAATGAGGAAATTGCATCGCATTGTCTGAGTAGGTGTCATTCTATTCT
GGGGGGTGGGGTGGGGCAGGACAGCAAGGGGGAGGATTGGGAAGACAATAGCAGGCATGCTGGG
GATGCGGTGGGCTCTATGGATTTGGCTACAGCAACAGGGTGGTGGACCTCATGGCCCACATGGC
CTCCAAGGAGTAAGACCCCTGGACCACCAGCCCCAGCAAGAGCACAAGAGGAAGAGAGAGACCC
TCACTGCTGGGGAGTCCCTGCCACACTCAGTCCCCCACCACACTGAATCTCCCCTCCTCACAGT
TGCCATGTAGACCCCTTGAAGAGGGGAGGGGCCTAGGGAGCCGCACCTTGTCATGTACCATCAA
TAAAGTACCCTGTGCTCAACCAGTTACTTGTCCTGTCTTATTCTAGGGTCTGGGGCAGAGGGGA
GGGAAGCTGGGCTTGTGTCAAGGTGAGACATTCTTGCTGGGGAGGGACCTGGTATGTTCTCCTC
AGACTGAGGGTAGGGCCTCCAAACAGCCTTGCTTGCTTCGAGAACCATTTGCTTCCCGCTCAGA
CGTCTTGAGTGCTACAGGAAGCTGGCACCACTACTTCAGAGAACAAGGCCTTTTCCTCTCCTCG
CTCCAGTAGATCTAGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGGCGGCCTCAGTGAG
CGAGCGAGCGCGCAGCTGCCTGCAGG
exemplary AAV construct for donor template insertion
at GAPDH locus
SEQ ID NO: 204
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGTCGACGAAGACTGTGGATGGCCCCTCCGGGAAACTGTGGCGTGATGGCCG
CGGGGCTCTCCAGAACATCATCCCTGCCTCTACTGGCGCTGCCAAGGCTGTGGGCAAGGTCATC
CCTGAGCTGAACGGGAAGCTCACTGGCATGGCCTTCCGTGTCCCCACTGCCAACGTGTCAGTGG
TGGACCTGACCTGCCGTCTAGAAAAACCTGCCAAATATGATGACATCAAGAAGGTGGTGAAGCA
GGCGTCGGAGGGCCCCCTCAAGGGCATCCTGGGCTACACTGAGCACCAGGTGGTCTCCTCTGAC
TTCAACAGCGACACCCACTCCTCCACCTTTGACGCTGGGGCTGGCATTGCCCTCAACGACCACT
TTGTCAAGCTCATTTCCTGGTATGTGGCTGGGGCCAGAGACTGGCTCTTAAAAAGTGCAGGGTC
TGGCGCCCTCTGGTGGCTGGCTCAGAAAAAGGGCCCTGACAACTCTTTACATCTTCTAGGTATG
ACAACGAGTTCGGATATAGCAATAGAGTGGTCGATCTGATGGCTCATATGGCTAGCAAAGAGGG
AAGCGGAGCTACTAACTTCAGCCTGCTGAAGCAGGCTGGAGACGTGGAGGAGAACCCTGGACCT
ATGGCACTCCCCGTCACCGCCCTTCTCTTGCCCCTCGCCCTGCTGCTGCATGCTGCCAGGCCCA
TGGACGAAGTGCAGCTCGTGGAGTCCGGTGGAGGACTCGTCCAACCGGGCGGATCCCTTCGCTT
GTCCTGCGCCGCATCAGGCTTCAGCTTCACCAACTATGGCGTCCACTGGGTCAGACAGGCCCCC
GGAAAGGGACTGGAATGGGTGTCCGTGATCTGGAGCGGCGGGAACACCGACTACAACACCTCCG
TGAAGGGCCGGTTCACTATTAGCCGCGACAACTCCAAGAACACTCTGTACCTCCAAATGAACTC
CCTGAGGGCCGAAGATACTGCTGTGTACTATTGCGCGAGAGCCCTGACCTACTACGACTACGAG
TTCGCGTACTGGGGCCAGGGGACTCTCGTGACCGTGTCCAGCGGTGGTGGAGGTTCCGGAGGCG
GAGGTTCTGGTGGCGGGGGATCAGAAATCGTGCTGACTCAGTCCCCTGCGACCTTGTCCCTGAG
CCCTGGAGAACGGGCCACCCTGAGCTGTAGAGCCAGCCAGAGCATCGGGACAAATATTCACTGG
TACCAGCAGAAACCCGGACAAGCACCACGGCTGCTGATCTACTACGCCTCCGAGTCGATTTCCG
GAATCCCGGCTCGCTTTTCGGGGTCTGGATCGGGAACGGACTTCACTCTGACCATCTCGTCGCT
GGAACCCGAGGATTTCGCCGTGTACTACTGCCAACAGAACAACAATTGGCCGACCACGTTCGGC
CAGGGCACCAAGCTCGAGATTAAGGGATCACTGGAAGCGGCCGCAACCACAACACCTGCTCCAA
GGCCCCCCACACCCGCTCCAACTATAGCCAGCCAACCATTGAGCCTCAGACCTGAAGCTTGCAG
GCCCGCAGCAGGAGGCGCCGTCCATACGCGAGGCCTGGACTTCGCGTGTGATATTTATATTTGG
GCCCCTTTGGCCGGAACATGTGGGGTGTTGCTTCTCTCCCTTGTGATCACTCTGTATTGTAAGC
GCGGGAGAAAGAAGCTCCTGTACATCTTCAAGCAGCCTTTTATGCGACCTGTGCAAACCACTCA
GGAAGAAGATGGGTGTTCATGCCGCTTCCCCGAGGAGGAAGAAGGAGGGTGTGAACTGAGGGTG
AAATTTTCTAGAAGCGCCGATGCTCCCGCATATCAGCAGGGTCAGAATCAGCTCTACAATGAAT
TGAATCTCGGCAGGCGAGAAGAGTACGATGTTCTGGACAAGAGACGGGGCAGGGATCCCGAGAT
GGGGGGAAAGCCCCGGAGAAAAAATCCTCAGGAGGGGTTGTACAATGAGCTGCAGAAGGACAAG
ATGGCTGAAGCCTATAGCGAGATCGGAATGAAAGGCGAAAGACGCAGAGGCAAGGGGCATGACG
GTCTGTACCAGGGTCTCTCTACAGCCACCAAGGACACTTATGATGCGTTGCATATGCAAGCCTT
GCCACCCCGCTAAAGCGGCCGCGTCGAGTCTAGAGGGCCCGTTTAAACCCGCTGATCAGCCTCG
ACTGTGCCTTCTAGTTGCCAGCCATCTGTTGTTTGCCCCTCCCCCGTGCCTTCCTTGACCCTGG
AAGGTGCCACTCCCACTGTCCTTTCCTAATAAAATGAGGAAATTGCATCGCATTGTCTGAGTAG
GTGTCATTCTATTCTGGGGGGTGGGGTGGGGCAGGACAGCAAGGGGGAGGATTGGGAAGACAAT
AGCAGGCATGCTGGGGATGCGGTGGGCTCTATGGATTTGGCTACAGCAACAGGGTGGTGGACCT
CATGGCCCACATGGCCTCCAAGGAGTAAGACCCCTGGACCACCAGCCCCAGCAAGAGCACAAGA
GGAAGAGAGAGACCCTCACTGCTGGGGAGTCCCTGCCACACTCAGTCCCCCACCACACTGAATC
TCCCCTCCTCACAGTTGCCATGTAGACCCCTTGAAGAGGGGAGGGGCCTAGGGAGCCGCACCTT
GTCATGTACCATCAATAAAGTACCCTGTGCTCAACCAGTTACTTGTCCTGTCTTATTCTAGGGT
CTGGGGCAGAGGGGAGGGAAGCTGGGCTTGTGTCAAGGTGAGACATTCTTGCTGGGGAGGGACC
TGGTATGTTCTCCTCAGACTGAGGGTAGGGCCTCCAAACAGCCTTGCTTGCTTCGAGAACCATT
TGCTTCCCGCTCAGACGTCTTGAGTGCTACAGGAAGCTGGCACCACTACTTCAGAGAACAAGGC
CTTTTCCTCTCCTCGCTCCAGTAGATCTAGGAACCCCTAGTGATGGAGTTGGCCACTCCCTCTC
TGCGCGCTCGCTCGCTCACTGAGGCCGGGCGACCAAAGGTCGCCCGACGCCCGGGCTTTGCCCG
GGCGGCCTCAGTGAGCGAGCGAGCGCGCAGCTGCCTGCAGG

Exemplary Donor Template Sequences

In some embodiments, a donor template comprises in 5′ to 3′ order, a target sequence 5′ homology arm (which optionally comprises an optimized sequence that is not a wild type sequence), a second regulatory element that enables expression of a cargo sequence as a separate translational product (e.g., an IRES sequence and/or a 2A element), a cargo sequence (e.g., a gene product of interest), optionally a second regulatory element that enables expression of a cargo sequence as a separate translational product (e.g., an IRES sequence and/or a 2A element), optionally a second cargo sequence (e.g., a gene product of interest), optionally a 3′ UTR, a poly adenylation signal (e.g., a BGHpA signal), and a target sequence 3′ homology arm (which optionally comprises an optimized sequence that is not a wild type sequence).

In some embodiments, a donor template comprises or consists of the sequence of any one of SEQ ID NOs: 38-57 and 205-218. In some embodiments, a donor template comprises or consists of a sequence that is at least 85%, 90%, 95%, 98% or 99% identical to any one of SEQ ID NOs: 38-57 and 205-218.

exemplary donor template for insertion at GAPDH locus
SEQ ID NO: 38
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGAGGGCAGAGGAAGTCTTCTA
ACATGCGGTGACGTGGAGGAGAATCCTGGCCCGATGGTGAGCAAGGGCGAGGAGCTGTTCACCG
GGGTGGTGCCCATCCTGGTCGAGCTGGACGGCGACGTAAACGGCCACAAGTTCAGCGTGTCCGG
CGAGGGCGAGGGCGATGCCACCTACGGCAAGCTGACCCTGAAGTTCATCTGCACCACCGGCAAG
CTGCCCGTGCCCTGGCCCACCCTCGTGACCACCCTGACCTACGGCGTGCAGTGCTTCAGCCGCT
ACCCCGACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACATCCTGG
GGCACAAGCTGGAGTACAACTACAACAGCCACAACGTCTATATCATGGCCGACAAGCAGAAGAA
CGGCATCAAGGTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAACCACTACCTGA
GCACCCAGTCCGCCCTGAGCAAAGACCCCAACGAGAAGCGCGATCACATGGTCCTGCTGGAGTT
CGTGACCGCCGCCGGGATCACTCTCGGCATGGACGAGCTGTACAAGGAGGGCAGAGGAAGTCTT
CTAACATGCGGTGACGTGGAGGAGAATCCTGGCCCGATGGTGAGCAAGGGCGAGGAGGATAACA
TGGCCATCATCAAGGAGTTCATGCGCTTCAAGGTGCACATGGAGGGCTCCGTGAACGGCCACGA
GTTCGAGATCGAGGGCGAGGGCGAGGGCCGCCCCTACGAGGGCACCCAGACCGCCAAGCTGAAG
GTGACCAAGGGTGGCCCCCTGCCCTTCGCCTGGGACATCCTGTCCCCTCAGTTCATGTACGGCT
CCAAGGCCTACGTGAAGCACCCCGCCGACATCCCCGACTACTTGAAGCTGTCCTTCCCCGAGGG
CTTCAAGTGGGAGCGCGTGATGAACTTCGAGGACGGCGGCGTGGTGACCGTGACCCAGGACTCC
TCCCTGCAGGACGGCGAGTTCATCTACAAGGTGAAGCTGCGCGGCACCAACTTCCCCTCCGACG
GCCCCGTAATGCAGAAGAAGACAATGGGCTGGGAGGCCTCCTCCGAGCGGATGTACCCCGAGGA
CGGCGCCCTGAAGGGCGAGATCAAGCAGAGGCTGAAGCTGAAGGACGGCGGCCACTACGACGCT
GAGGTCAAGACCACCTACAAGGCCAAGAAGCCCGTGCAGCTGCCCGGCGCCTACAACGTCAACA
TCAAGTTGGACATCACCTCCCACAACGAGGACTACACCATCGTGGAACAGTACGAACGCGCCGA
GGGCCGCCACTCCACCGGCGGCATGGACGAGCTGTACAAGTAAGCGGCCGCGTCGAGTCTAGAG
GGCCCGTTTAAACCCGCTGATCAGCCTCGACTGTGCCTTCTAGTTGCCAGCCATCTGTTGTTTG
CCCCTCCCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTGGGGCAGG
ACAGCAAGGGGGAGGATTGGGAAGACAATAGCAGGCATGCTGGGGATGCGGTGGGCTCTATGGA
TTTGGCTACAGCAACAGGGTGGTGGACCTCATGGCCCACATGGCCTCCAAGGAGTAAGACCCCT
GGACCACCAGCCCCAGCAAGAGCACAAGAGGAAGAGAGAGACCCTCACTGCTGGGGAGTCCCTG
CCACACTCAGTCCCCCACCACACTGAATCTCCCCTCCTCACAGTTGCCATGTAGACCCCTTGAA
GAGGGGAGGGGCCTAGGGAGCCGCACCTTGTCATGTACCATCAATAAAGTACCCTGTGCTCAAC
CAGTTACTTGTCCTGTCTTATTCTAGGGTCTGGGGCAGAGGGGAGGGAAGCTGGGCTTGTGTCA
AGGTGAGACATTCTTGCTGGGGAGGGACCTGGTATGTTCTCCTCAGACTGAGGGTAGGGCCTCC
AAACAGCCTTGCTTGCTTCGAGAACCATTTGCTTCCCGCTCAGACGTCTTGAGTGCTACAGGAA
GCTGGCACCACTACTTCAGAGAACAAGGCCTTTTCCTCTCCTCGCTCCAGT
exemplary donor template for insertion at GAPDH locus
SEQ ID NO: 39
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGTGAGCAAGGGCGAGG
AGCTGTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACG
GCAACATCCTGGGGCACAAGCTGGAGTACAACTACAACAGCCACAACGTCTATATCATGGCCGA
CAAGCAGAAGAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCGATCACATGGT
CCTGCTGGAGTTCGTGACCGCCGCCGGGATCACTCTCGGCATGGACGAGCTGTACAAGTAACCC
CTCTCCCTCCCCCCCCCCTAACGTTACTGGCCGAAGCCGCTTGGAATAAGGCCGGTGTGCGTTT
GTCTATATGTTATTTTCCACCATATTGCCGTCTTTTGGCAATGTGAGGGCCCGGAAACCTGGCC
CTGTCTTCTTGACGAGCATTCCTAGGGGTCTTTCCCCTCTCGCCAAAGGAATGCAAGGTCTGTT
GAATGTCGTGAAGGAAGCAGTTCCTCTGGAAGCTTCTTGAAGACAAACAACGTCTGTAGCGACC
CTTTGCAGGCAGCGGAACCCCCCACCTGGCGACAGGTGCCTCTGCGGCCAAAAGCCACGTGTAT
AAGATACACCTGCAAAGGCGGCACAACCCCAGTGCCACGTTGTGAGTTGGATAGTTGTGGAAAG
AGTCAAATGGCTCTCCTCAAGCGTATTCAACAAGGGGCTGAAGGATGCCCAGAAGGTACCCCAT
TGTATGGGATCTGATCTGGGGCCTCGGTGCACATGCTTTACATGTGTTTAGTCGAGGTTAAAAA
AACGTCTAGGCCCCCCGAACCACGGGGACGTGGTTTTCCTTTGAAAAACACGATGATAAATGGT
GAGCAAGGGCGAGGAGGATAACATGGCCATCATCAAGGAGTTCATGCGCTTCAAGGTGCACATG
GAGGGCTCCGTGAACGGCCACGAGTTCGAGATCGAGGGCGAGGGCGAGGGCCGCCCCTACGAGG
GCACCCAGACCGCCAAGCTGAAGGTGACCAAGGGTGGCCCCCTGCCCTTCGCCTGGGACATCCT
GTCCCCTCAGTTCATGTACGGCTCCAAGGCCTACGTGAAGCACCCCGCCGACATCCCCGACTAC
TTGAAGCTGTCCTTCCCCGAGGGCTTCAAGTGGGAGCGCGTGATGAACTTCGAGGACGGCGGCG
TGGTGACCGTGACCCAGGACTCCTCCCTGCAGGACGGCGAGTTCATCTACAAGGTGAAGCTGCG
CGGCACCAACTTCCCCTCCGACGGCCCCGTAATGCAGAAGAAGACAATGGGCTGGGAGGCCTCC
TCCGAGCGGATGTACCCCGAGGACGGCGCCCTGAAGGGCGAGATCAAGCAGAGGCTGAAGCTGA
AGGACGGCGGCCACTACGACGCTGAGGTCAAGACCACCTACAAGGCCAAGAAGCCCGTGCAGCT
GCCCGGCGCCTACAACGTCAACATCAAGTTGGACATCACCTCCCACAACGAGGACTACACCATC
GTGGAACAGTACGAACGCGCCGAGGGCCGCCACTCCACCGGCGGCATGGACGAGCTGTACAAGT
AAGCGGCCGCGTCGAGTCTAGAGGGCCCGTTTAAACCCGCTGATCAGCCTCGACTGTGCCTTCT
AGTTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAATAGCAGGCATGCT
GGGGATGCGGTGGGCTCTATGGATTTGGCTACAGCAACAGGGTGGTGGACCTCATGGCCCACAT
GGCCTCCAAGGAGTAAGACCCCTGGACCACCAGCCCCAGCAAGAGCACAAGAGGAAGAGAGAGA
CCCTCACTGCTGGGGAGTCCCTGCCACACTCAGTCCCCCACCACACTGAATCTCCCCTCCTCAC
AGTTGCCATGTAGACCCCTTGAAGAGGGGAGGGGCCTAGGGAGCCGCACCTTGTCATGTACCAT
CAATAAAGTACCCTGTGCTCAACCAGTTACTTGTCCTGTCTTATTCTAGGGTCTGGGGCAGAGG
GGAGGGAAGCTGGGCTTGTGTCAAGGTGAGACATTCTTGCTGGGGAGGGACCTGGTATGTTCTC
CTCAGACTGAGGGTAGGGCCTCCAAACAGCCTTGCTTGCTTCGAGAACCATTTGCTTCCCGCTC
AGACGTCTTGAGTGCTACAGGAAGCTGGCACCACTACTTCAGAGAACAAGGCCTTTTCCTCTCC
TCGCTCCAGT
exemplary donor template for insertion at GAPDH locus
SEQ ID NO: 40
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGTGAGCAAGGGCGAGG
AGCTGTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACG
GCAACATCCTGGGGCACAAGCTGGAGTACAACTACAACAGCCACAACGTCTATATCATGGCCGA
CAAGCAGAAGAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCGATCACATGGT
CCTGCTGGAGTTCGTGACCGCCGCCGGGATCACTCTCGGCATGGACGAGCTGTACAAGGGAAGC
GGAGCTACTAACTTCAGCCTGCTGAAGCAGGCTGGAGACGTGGAGGAGAACCCTGGACCTATGG
TGAGCAAGGGCGAGGAGGATAACATGGCCATCATCAAGGAGTTCATGCGCTTCAAGGTGCACAT
GGAGGGCTCCGTGAACGGCCACGAGTTCGAGATCGAGGGCGAGGGCGAGGGCCGCCCCTACGAG
GGCACCCAGACCGCCAAGCTGAAGGTGACCAAGGGTGGCCCCCTGCCCTTCGCCTGGGACATCC
TGTCCCCTCAGTTCATGTACGGCTCCAAGGCCTACGTGAAGCACCCCGCCGACATCCCCGACTA
CTTGAAGCTGTCCTTCCCCGAGGGCTTCAAGTGGGAGCGCGTGATGAACTTCGAGGACGGCGGC
GTGGTGACCGTGACCCAGGACTCCTCCCTGCAGGACGGCGAGTTCATCTACAAGGTGAAGCTGC
GCGGCACCAACTTCCCCTCCGACGGCCCCGTAATGCAGAAGAAGACAATGGGCTGGGAGGCCTC
CTCCGAGCGGATGTACCCCGAGGACGGCGCCCTGAAGGGCGAGATCAAGCAGAGGCTGAAGCTG
AAGGACGGCGGCCACTACGACGCTGAGGTCAAGACCACCTACAAGGCCAAGAAGCCCGTGCAGC
TGCCCGGCGCCTACAACGTCAACATCAAGTTGGACATCACCTCCCACAACGAGGACTACACCAT
CGTGGAACAGTACGAACGCGCCGAGGGCCGCCACTCCACCGGCGGCATGGACGAGCTGTACAAG
TAAGCGGCCGCGTCGAGTCTAGAGGGCCCGTTTAAACCCGCTGATCAGCCTCGACTGTGCCTTC
TAGTTGCCAGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGTGTCATTCTA
TTCTGGGGGGTGGGGTGGGGCAGGACAGCAAGGGGGAGGATTGGGAAGACAATAGCAGGCATGC
TGGGGATGCGGTGGGCTCTATGGATTTGGCTACAGCAACAGGGTGGTGGACCTCATGGCCCACA
TGGCCTCCAAGGAGTAAGACCCCTGGACCACCAGCCCCAGCAAGAGCACAAGAGGAAGAGAGAG
ACCCTCACTGCTGGGGAGTCCCTGCCACACTCAGTCCCCCACCACACTGAATCTCCCCTCCTCA
CAGTTGCCATGTAGACCCCTTGAAGAGGGGAGGGGCCTAGGGAGCCGCACCTTGTCATGTACCA
TCAATAAAGTACCCTGTGCTCAACCAGTTACTTGTCCTGTCTTATTCTAGGGTCTGGGGCAGAG
GGGAGGGAAGCTGGGCTTGTGTCAAGGTGAGACATTCTTGCTGGGGAGGGACCTGGTATGTTCT
CCTCAGACTGAGGGTAGGGCCTCCAAACAGCCTTGCTTGCTTCGAGAACCATTTGCTTCCCGCT
CAGACGTCTTGAGTGCTACAGGAAGCTGGCACCACTACTTCAGAGAACAAGGCCTTTTCCTCTC
CTCGCTCCAGT
exemplary donor template for insertion at GAPDH locus
SEQ ID NO: 41
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGTGAGCAAGGGCGAGG
AGCTGTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACG
GCAACATCCTGGGGCACAAGCTGGAGTACAACTACAACAGCCACAACGTCTATATCATGGCCGA
CAAGCAGAAGAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCGATCACATGGT
CCTGCTGGAGTTCGTGACCGCCGCCGGGATCACTCTCGGCATGGACGAGCTGTACAAGGAGGGC
AGAGGAAGTCTTCTAACATGCGGTGACGTGGAGGAGAATCCTGGCCCGATGGTGAGCAAGGGCG
AGGAGGATAACATGGCCATCATCAAGGAGTTCATGCGCTTCAAGGTGCACATGGAGGGCTCCGT
GAACGGCCACGAGTTCGAGATCGAGGGCGAGGGCGAGGGCCGCCCCTACGAGGGCACCCAGACC
GCCAAGCTGAAGGTGACCAAGGGTGGCCCCCTGCCCTTCGCCTGGGACATCCTGTCCCCTCAGT
TCATGTACGGCTCCAAGGCCTACGTGAAGCACCCCGCCGACATCCCCGACTACTTGAAGCTGTC
CTTCCCCGAGGGCTTCAAGTGGGAGCGCGTGATGAACTTCGAGGACGGCGGCGTGGTGACCGTG
ACCCAGGACTCCTCCCTGCAGGACGGCGAGTTCATCTACAAGGTGAAGCTGCGCGGCACCAACT
TCCCCTCCGACGGCCCCGTAATGCAGAAGAAGACAATGGGCTGGGAGGCCTCCTCCGAGCGGAT
GTACCCCGAGGACGGCGCCCTGAAGGGCGAGATCAAGCAGAGGCTGAAGCTGAAGGACGGCGGC
CACTACGACGCTGAGGTCAAGACCACCTACAAGGCCAAGAAGCCCGTGCAGCTGCCCGGCGCCT
ACAACGTCAACATCAAGTTGGAGATCACCTCCCACAACGAGGACTAGACCATCGTGGAACAGTA
CGAACGCGCCGAGGGCCGCCACTCCACCGGCGGCATGGACGAGCTGTACAAGTAAGCGGCCGCG
TCGAGTCTAGAGGGCCCGTTTAAACCCGCTGATCAGCCTCGACTGTGCCTTCTAGTTGCCAGCC
ATCTGTTGTTTGCCCCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTCTATTCTGGGGGGTG
GGGTGGGGCAGGACAGCAAGGGGGAGGATTGGGAAGACAATAGCAGGCATGCTGGGGATGCGGT
GGGCTCTATGGATTTGGCTACAGCAACAGGGTGGTGGACCTCATGGCCCACATGGCCTCCAAGG
AGTAAGACCCCTGGACCACCAGCCCCAGCAAGAGCACAAGAGGAAGAGAGAGACCCTCACTGCT
GGGGAGTCCCTGCCACACTCAGTCCCCCACCACACTGAATCTCCCCTCCTCACAGTTGCCATGT
AGACCCCTTGAAGAGGGGAGGGGCCTAGGGAGCCGCACCTTGTCATGTACCATCAATAAAGTAC
CCTGTGCTCAACCAGTTACTTGTCCTGTCTTATTCTAGGGTCTGGGGCAGAGGGGAGGGAAGCT
GGGCTTGTGTCAAGGTGAGACATTCTTGCTGGGGAGGGACCTGGTATGTTCTCCTCAGACTGAG
GGTAGGGCCTCCAAACAGCCTTGCTTGCTTCGAGAACCATTTGCTTCCCGCTCAGACGTCTTGA
GTGCTACAGGAAGCTGGCACCACTACTTCAGAGAACAAGGCCTTTTCCTCTCCTCGCTCCAGT
exemplary donor template for insertion at GAPDH locus
SEQ ID NO: 42
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGAGGGCAGAGGAAGTCTTCTA
ACATGCGGTGACGTGGAGGAGAATCCTGGCCCGATGGTGAGCAAGGGCGAGGAGCTGTTCACCG
GGGTGGTGCCCATCCTGGTCGAGCTGGACGGCGACGTAAACGGCCACAAGTTCAGCGTGTCCGG
CGAGGGCGAGGGCGATGCCACCTACGGCAAGCTGACCCTGAAGTTCATCTGCACCACCGGCAAG
CTGCCCGTGCCCTGGCCCACCCTCGTGACCACCCTGACCTACGGCGTGCAGTGCTTCAGCCGCT
ACCCCGACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACATCCTGG
GGCACAAGCTGGAGTACAACTACAACAGCCACAACGTCTATATCATGGCCGACAAGCAGAAGAA
CGGCATCAAGGTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAACCACTACCTGA
GCACCCAGTCCGCCCTGAGCAAAGACCCCAACGAGAAGCGCGATCACATGGTCCTGCTGGAGTT
CGTGACCGCCGCCGGGATCACTCTCGGCATGGACGAGCTGTACAAGGGAAGCGGAGCTACTAAC
TTCAGCCTGCTGAAGCAGGCTGGAGACGTGGAGGAGAACCCTGGACCTATGGTGAGCAAGGGCG
AGGAGGATAACATGGCCATCATCAAGGAGTTCATGCGCTTCAAGGTGCACATGGAGGGCTCCGT
GAACGGCCACGAGTTCGAGATCGAGGGCGAGGGCGAGGGCCGCCCCTACGAGGGCACCCAGACC
GCCAAGCTGAAGGTGACCAAGGGTGGCCCCCTGCCCTTCGCCTGGGACATCCTGTCCCCTCAGT
TCATGTACGGCTCCAAGGCCTACGTGAAGCACCCCGCCGACATCCCCGACTACTTGAAGCTGTC
CTTCCCCGAGGGCTTCAAGTGGGAGCGCGTGATGAACTTCGAGGACGGCGGCGTGGTGACCGTG
ACCCAGGACTCCTCCCTGCAGGACGGCGAGTTCATCTACAAGGTGAAGCTGCGCGGCACCAACT
TCCCCTCCGACGGCCCCGTAATGCAGAAGAAGACAATGGGCTGGGAGGCCTCCTCCGAGCGGAT
GTACCCCGAGGACGGCGCCCTGAAGGGCGAGATCAAGCAGAGGCTGAAGCTGAAGGACGGCGGC
CACTACGACGCTGAGGTCAAGACCACCTACAAGGCCAAGAAGCCCGTGCAGCTGCCCGGCGCCT
ACAACGTCAACATCAAGTTGGAGATCACCTCCCACAACGAGGACTAGACCATCGTGGAACAGTA
CGAACGCGCCGAGGGCCGCCACTCCACCGGCGGCATGGACGAGCTGTACAAGTAAGCGGCCGCG
TCGAGTCTAGAGGGCCCGTTTAAACCCGCTGATCAGCCTCGACTGTGCCTTCTAGTTGCCAGCC
ATCTGTTGTTTGCCCCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTCTATTCTGGGGGGTG
GGGTGGGGCAGGACAGCAAGGGGGAGGATTGGGAAGACAATAGCAGGCATGCTGGGGATGCGGT
GGGCTCTATGGATTTGGCTACAGCAACAGGGTGGTGGACCTCATGGCCCACATGGCCTCCAAGG
AGTAAGACCCCTGGACCACCAGCCCCAGCAAGAGCACAAGAGGAAGAGAGAGACCCTCACTGCT
GGGGAGTCCCTGCCACACTCAGTCCCCCACCACACTGAATCTCCCCTCCTCACAGTTGCCATGT
AGACCCCTTGAAGAGGGGAGGGGCCTAGGGAGCCGCACCTTGTCATGTACCATCAATAAAGTAC
CCTGTGCTCAACCAGTTACTTGTCCTGTCTTATTCTAGGGTCTGGGGCAGAGGGGAGGGAAGCT
GGGCTTGTGTCAAGGTGAGACATTCTTGCTGGGGAGGGACCTGGTATGTTCTCCTCAGACTGAG
GGTAGGGCCTCCAAACAGCCTTGCTTGCTTCGAGAACCATTTGCTTCCCGCTCAGACGTCTTGA
GTGCTACAGGAAGCTGGCACCACTACTTCAGAGAACAAGGCCTTTTCCTCTCCTCGCTCCAGT
exemplary donor template for insertion at GAPDH locus
SEQ ID NO: 43
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGAGGGCAGAGGAAGTCTTCTA
ACATGCGGTGACGTGGAGGAGAATCCTGGCCCGATGGTGAGCAAGGGCGAGGAGCTGTTCACCG
GGGTGGTGCCCATCCTGGTCGAGCTGGACGGCGACGTAAACGGCCACAAGTTCAGCGTGTCCGG
CGAGGGCGAGGGCGATGCCACCTACGGCAAGCTGACCCTGAAGTTCATCTGCACCACCGGCAAG
CTGCCCGTGCCCTGGCCCACCCTCGTGACCACCCTGACCTACGGCGTGCAGTGCTTCAGCCGCT
ACCCCGACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACATCCTGG
GGCACAAGCTGGAGTACAACTACAACAGCCACAACGTCTATATCATGGCCGACAAGCAGAAGAA
CGGCATCAAGGTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAACCACTACCTGA
GCACCCAGTCCGCCCTGAGCAAAGACCCCAACGAGAAGCGCGATCACATGGTCCTGCTGGAGTT
CGTGACCGCCGCCGGGATCACTCTCGGCATGGACGAGCTGTACAAGTAACCCCTCTCCCTCCCC
CCCCCCTAACGTTACTGGCCGAAGCCGCTTGGAATAAGGCCGGTGTGCGTTTGTCTATATGTTA
TTTTCCACCATATTGCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTGTTGAATGTCGTGAA
GGAAGCAGTTCCTCTGGAAGCTTCTTGAAGACAAACAACGTCTGTAGCGACCCTTTGCAGGCAG
CGGAACCCCCCACCTGGCGACAGGTGCCTCTGCGGCCAAAAGCCACGTGTATAAGATACACCTG
CAAAGGCGGCACAACCCCAGTGCCACGTTGTGAGTTGGATAGTTGTGGAAAGAGTCAAATGGCT
CTCCTCAAGCGTATTCAACAAGGGGCTGAAGGATGCCCAGAAGGTACCCCATTGTATGGGATCT
GATCTGGGGCCTCGGTGCACATGCTTTACATGTGTTTAGTCGAGGTTAAAAAAACGTCTAGGCC
CCCCGAACCACGGGGACGTGGTTTTCCTTTGAAAAACACGATGATAAATGGTGAGCAAGGGCGA
GGAGGATAACATGGCCATCATCAAGGAGTTCATGCGCTTCAAGGTGCACATGGAGGGCTCCGTG
AACGGCCACGAGTTCGAGATCGAGGGCGAGGGCGAGGGCCGCCCCTACGAGGGCACCCAGACCG
CCAAGCTGAAGGTGACCAAGGGTGGCCCCCTGCCCTTCGCCTGGGACATCCTGTCCCCTCAGTT
CATGTACGGCTCCAAGGCCTACGTGAAGCACCCCGCCGACATCCCCGACTACTTGAAGCTGTCC
TTCCCCGAGGGCTTCAAGTGGGAGCGCGTGATGAACTTCGAGGACGGCGGCGTGGTGACCGTGA
CCCAGGACTCCTCCCTGCAGGACGGCGAGTTCATCTACAAGGTGAAGCTGCGCGGCACCAACTT
CCCCTCCGACGGCCCCGTAATGCAGAAGAAGACAATGGGCTGGGAGGCCTCCTCCGAGCGGATG
TACCCCGAGGACGGCGCCCTGAAGGGCGAGATCAAGCAGAGGCTGAAGCTGAAGGACGGCGGCC
ACTACGACGCTGAGGTCAAGACCACCTACAAGGCCAAGAAGCCCGTGCAGCTGCCCGGCGCCTA
CAACGTCAACATCAAGTTGGAGATCACCTCCCACAACGAGGAGTAGACCATCGTGGAACAGTAG
GAACGCGCCGAGGGCCGCCACTCCACCGGCGGCATGGACGAGCTGTACAAGTAAGCGGCCGCGT
CGAGTCTAGAGGGCCCGTTTAAACCCGCTGATCAGCCTCGACTGTGCCTTCTAGTTGCCAGCCA
TCTGTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGGGTGG
GGTGGGGCAGGACAGCAAGGGGGAGGATTGGGAAGACAATAGCAGGCATGCTGGGGATGCGGTG
GGCTCTATGGATTTGGCTACAGCAACAGGGTGGTGGACCTCATGGCCCACATGGCCTCCAAGGA
GTAAGACCCCTGGACCACCAGCCCCAGCAAGAGCACAAGAGGAAGAGAGAGACCCTCACTGCTG
GGGAGTCCCTGCCACACTCAGTCCCCCACCACACTGAATCTCCCCTCCTCACAGTTGCCATGTA
GACCCCTTGAAGAGGGGAGGGGCCTAGGGAGCCGCACCTTGTCATGTACCATCAATAAAGTACC
CTGTGCTCAACCAGTTACTTGTCCTGTCTTATTCTAGGGTCTGGGGCAGAGGGGAGGGAAGCTG
GGCTTGTGTCAAGGTGAGACATTCTTGCTGGGGAGGGACCTGGTATGTTCTCCTCAGACTGAGG
GTAGGGCCTCCAAACAGCCTTGCTTGCTTCGAGAACCATTTGCTTCCCGCTCAGACGTCTTGAG
TGCTACAGGAAGCTGGCACCACTACTTCAGAGAACAAGGCCTTTTCCTCTCCTCGCTCCAGT
exemplary donor template for insertion at GAPDH locus
SEQ ID NO: 44
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGTGAGCAAGGGCGAGG
AGCTGTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACG
GCAACATCCTGGGGCACAAGCTGGAGTACAACTACAACAGCCACAACGTCTATATCATGGCCGA
CAAGCAGAAGAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCGATCACATGGT
CCTGCTGGAGTTCGTGACCGCCGCCGGGATCACTCTCGGCATGGACGAGCTGTACAAGTGAGCG
GCCGCGTCGAGTCTAGAGGGCCCGTTTAAACCCGCTGATCAGCCTCGACTGTGCCTTCTAGTTG
CCAGCCATCTGTTGTTTGCCCCTCCCCCGTGCCTTCCTTGACCCTGGAAGGTGCCACTCCCACT
GTCCTTTCCTAATAAAATGAGGAAATTGCATCGCATTGTCTGAGTAGGTGTCATTCTATTCTGG
GGGGTGGGGTGGGGCAGGACAGCAAGGGGGAGGATTGGGAAGACAATAGCAGGCATGCTGGGGA
TGCGGTGGGCTCTATGGATTTGGCTACAGCAACAGGGTGGTGGACCTCATGGCCCACATGGCCT
CCAAGGAGTAAGACCCCTGGACCACCAGCCCCAGCAAGAGCACAAGAGGAAGAGAGAGACCCTC
ACTGCTGGGGAGTCCCTGCCACACTCAGTCCCCCACCACACTGAATCTCCCCTCCTCACAGTTG
CCATGTAGACCCCTTGAAGAGGGGAGGGGCCTAGGGAGCCGCACCTTGTCATGTACCATCAATA
AAGTACCCTGTGCTCAACCAGTTACTTGTCCTGTCTTATTCTAGGGTCTGGGGCAGAGGGGAGG
GAAGCTGGGCTTGTGTCAAGGTGAGACATTCTTGCTGGGGAGGGACCTGGTATGTTCTCCTCAG
ACTGAGGGTAGGGCCTCCAAACAGCCTTGCTTGCTTCGAGAACCATTTGCTTCCCGCTCAGACG
TCTTGAGTGCTACAGGAAGCTGGCACCACTACTTCAGAGAACAAGGCCTTTTCCTCTCCTCGCT
CCAGT
exemplary donor template for insertion at GAPDH locus
SEQ ID NO: 45
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGATTGGACCTGGATCC
TGTTTCTGGTGGCCGCTGCCACAAGAGTGCACAGCAATTGGGTCAACGTGATCAGCGACCTGAA
GAAGATCGAGGAGCTGATCCAGAGCATGCACATCGAGGCCACACTGTAGACCGAGTCCGATGTG
CACCCTAGCTGCAAAGTGACCGCCATGAAGTGCTTTCTGCTGGAACTGCAAGTGATCAGCCTGG
AAAGCGGCGACGCCAGCATCCACGATACCGTGGAAAACCTGATCATCCTGGCCAACAACAGCCT
GAGCAGCAACGGCAATGTGACCGAGAGCGGCTGCAAAGAGTGCGAGGAACTGGAAGAGAAGAAC
ATCAAAGAGTTCCTCCAGAGCTTCGTCCACATCGTGCAGATGTTCATCAACACCAGCTCTGGCG
GAGGAAGCGGAGGCGGAGGATCTGGTGGTGGTGGATCTGGCGGCGGTGGTAGTGGCGGAGGTTC
TCTGCAAATCACCTGTCCTCCACCTATGAGCGTGGAACACGCCGACATCTGGGTCAAGAGCTAC
AGCCTGTACAGCAGAGAGCGGTACATCTGCAACAGCGGCTTCAAGAGAAAGGCCGGCACAAGCA
GCCTGACCGAGTGTGTGCTGAACAAGGCCACAAACGTGGCCCACTGGACCACACCTAGCCTGAA
GTGCATCAGAGATCCCGCTCTGGTTCATCAGAGGCCTGCCCCTCCATCTACAGTGACAACAGCT
GGCGTGACCCCTCAGCCTGAGTCTCTGTCTCCATCTGGAAAAGAGCCTGCCGCCAGCTCTCCCA
GCTCTAACAATACTGCTGCCACCACAGCCGCTATCGTGCCTGGATCTCAGCTGATGCCTAGCAA
GAGCCCTAGCACCGGCACAACAGAGATCAGCTCTCACGAGAGCAGCCACGGAACACCTTCTCAG
ACCACCGCCAAGAATTGGGAGCTGACAGCCTCTGCCTCTCATCAGCCACCTGGCGTGTACCCAC
AGGGCCACTCTGATACAACAGTGGCCATCAGCACCAGCACCGTTCTGCTGTGTGGCCTGTCTGC
TGTTAGCCTGCTGGCCTGCTACCTGAAGTCTAGACAGACACCTCCTCTGGCCAGCGTGGAAATG
GAAGCCATGGAAGCTCTGCCTGTCACATGGGGCACCAGCAGCAGAGATGAGGACCTCGAGAATT
GCAGCCACCACCTGTAGGCGGCCGCGTCGAGTCTAGAGGGCCCGTTTAAACCCGCTGATCAGCC
TCGACTGTGCCTTCTAGTTGCCAGCCATCTGTTGTTTGCCCCTCCCCCGTGCCTTCCTTGACCC
TGGAAGGTGCCACTCCCACTGTCCTTTCCTAATAAAATGAGGAAATTGCATCGCATTGTCTGAG
TAGGTGTCATTCTATTCTGGGGGGTGGGGTGGGGCAGGACAGCAAGGGGGAGGATTGGGAAGAC
AATAGCAGGCATGCTGGGGATGCGGTGGGCTCTATGGATTTGGCTACAGCAACAGGGTGGTGGA
CCTCATGGCCCACATGGCCTCCAAGGAGTAAGACCCCTGGACCACCAGCCCCAGCAAGAGCACA
AGAGGAAGAGAGAGACCCTCACTGCTGGGGAGTCCCTGCCACACTCAGTCCCCCACCACACTGA
ATCTCCCCTCCTCACAGTTGCCATGTAGACCCCTTGAAGAGGGGAGGGGCCTAGGGAGCCGCAC
CTTGTCATGTACCATCAATAAAGTACCCTGTGCTCAACCAGTTACTTGTCCTGTCTTATTCTAG
GGTCTGGGGCAGAGGGGAGGGAAGCTGGGCTTGTGTCAAGGTGAGACATTCTTGCTGGGGAGGG
ACCTGGTATGTTCTCCTCAGACTGAGGGTAGGGCCTCCAAACAGCCTTGCTTGCTTCGAGAACC
ATTTGCTTCCCGCTCAGACGTCTTGAGTGCTACAGGAAGCTGGCACCACTACTTCAGAGAACAA
GGCCTTTTCCTCTCCTCGCTCCAGT
exemplary donor template for insertion at GAPDH locus
SEQ ID NO: 46
GGCTTTCCCATAATTTCCTTTCAAGGTGGGGAGGGAGGTAGAGGGGTGATGTGGGGAGTACGCT
GCAGGGCCTCACTCCTTTTGCAGACCACAGTCCATGCCATCACTGCCACCCAGAAGACTGTGGA
TGGCCCCTCCGGGAAACTGTGGCGTGATGGCCGCGGGGCTCTCCAGAACATCATCCCTGCCTCT
ACTGGCGCTGCCAAGGCTGTGGGCAAGGTCATCCCTGAGCTGAACGGGAAGCTCACTGGCATGG
CCTTCCGTGTCCCCACTGCCAACGTGTCAGTGGTGGACCTGACCTGCCGTCTAGAAAAACCTGC
CAAATATGATGACATCAAGAAGGTGGTGAAGCAGGCGTCGGAGGGCCCCCTCAAGGGCATCCTG
GGCTACACTGAGCACCAGGTGGTCTCCTCTGACTTCAACAGCGACACCCACTCCTCCACCTTTG
ACGCTGGGGCTGGCATTGCCCTCAACGACCACTTTGTCAAGCTCATCTCTTGGTACGACAATGA
GTTCGGATATAGCAATAGAGTGGTCGATCTGATGGCTCATATGGCTAGCAAAGAGGGAAGCGGA
GCTACTAACTTCAGCCTGCTGAAGCAGGCTGGAGACGTGGAGGAGAACCCTGGACCTATGGTGA
GCAAGGGCGAGGAGCTGTTCACCGGGGTGGTGCCCATCCTGGTCGAGCTGGACGGCGACGTAAA
CGGCCACAAGTTCAGCGTGTCCGGCGAGGGCGAGGGCGATGCCACCTACGGCAAGCTGACCCTG
AAGTTCATCTGCACCACCGGCAAGCTGCCCGTGCCCTGGCCCACCCTCGTGACCACCCTGACCT
ACGGCGTGCAGTGCTTCAGCCGCTACCCCGACCACATGAAGCAGCACGACTTCTTCAAGTCCGC
CATGCCCGAAGGCTACGTCCAGGAGCGCACCATCTTCTTCAAGGACGACGGCAACTACAAGACC
CGCGCCGAGGTGAAGTTCGAGGGCGACACCCTGGTGAACCGCATCGAGCTGAAGGGCATCGACT
TCAAGGAGGACGGCAACATCCTGGGGCACAAGCTGGAGTACAACTACAACAGCCACAACGTCTA
TATCATGGCCGACAAGCAGAAGAACGGCATCAAGGTGAACTTCAAGATCCGCCACAACATCGAG
GACGGCAGCGTGCAGCTCGCCGACCACTACCAGCAGAACACCCCCATCGGCGACGGCCCCGTGC
TGCTGCCCGACAACCACTACCTGAGCACCCAGTCCGCCCTGAGCAAAGACCCCAACGAGAAGCG
CGATCACATGGTCCTGCTGGAGTTCGTGACCGCCGCCGGGATCACTCTCGGCATGGACGAGCTG
TACAAGTGAGCGGCCGCGTCGAGTCTAGAGGGCCCGTTTAAACCCGCTGATCAGCCTCGACTGT
GCCTTCTAGTTGCCAGCCATCTGTTGTTTGCCCCTCCCCCGTGCCTTCCTTGACCCTGGAAGGT
GCCACTCCCACTGTCCTTTCCTAATAAAATGAGGAAATTGCATCGCATTGTCTGAGTAGGTGTC
ATTCTATTCTGGGGGGTGGGGTGGGGCAGGACAGCAAGGGGGAGGATTGGGAAGACAATAGCAG
GCATGCTGGGGATGCGGTGGGCTCTATGGAGACTGGCTCTTAAAAAGTGCAGGGTCTGGCGCCC
TCTGGTGGCTGGCTCAGAAAAAGGGCCCTGACAACTCTTTTCATCTTCTAGGTATGACAACGAA
TTTGGCTACAGCAACAGGGTGGTGGACCTCATGGCCCACATGGCCTCCAAGGAGTAAGACCCCT
GGACCACCAGCCCCAGCAAGAGCACAAGAGGAAGAGAGAGACCCTCACTGCTGGGGAGTCCCTG
CCACACTCAGTCCCCCACCACACTGAATCTCCCCTCCTCACAGTTGCCATGTAGACCCCTTGAA
GAGGGGAGGGGCCTAGGGAGCCGCACCTTGTCATGTACCATCAATAAAGTACCCTGTGCTCAAC
CAGTTACTTGTCCTGTCTTATTCTAGGGTCTGGGGCAGAGGGGAGGGAAGCTGGGCTTGTGTCA
AGGTGAGACATTCTTGCTGGGGAGGGACCTGGTATGTTCTCCTCAGACTGAGGGTAGGGCCTCC
AAACAGCCTTGCTTGCT
exemplary donor template for insertion at TBP locus
SEQ ID NO: 47
GCAGACTTCCATTTACAGTGAGGAGGTGAGCATTGCATTGAACAAAAGATGGCGTTTTCACTTG
GAATTAGTTATCTGAAGCTTTAGGATTCCTCAGCAATATGATTATGAGACAAGAAAGGAAGATT
CAGAAATGAGTCTAGTTGAAGGGAGCAATTCAGAGAAGAAGATTGAGTTGTTATCATTGCCGTC
CTGCTTGGTTTATGGCCTGGTTCAGGACCAAGGAGAGAAGTGTGAATACATGCCTCTTGAGCTA
TAGAATGAGACGCTGGAGTGAGTAAGATGATTTTTTAAAAGTATTGTTTTATAAACAAAAATAA
GATTGTGACAAGGGATTCCACTATTAATGTTTTCATGCCTGTGCCTTAATCTGACTGGGTATGG
TGAGAATTGTGCTTGCAGCTTTAAGGTAAGAATTTTACCATCTTAATATGTTAAGAAGTGCCAT
TTCAGTCTCTCATCTCTACTCCAACTTGTCTTCTTAGGTGCTAAAGTCAGAGCCGAAATCTACG
AGGCCTTCGAGAACATCTACCCCATCCTGAAGGGCTTCAGAAAGACCACCGGAAGCGGAGCTAC
TAACTTCAGCCTGCTGAAGCAGGCTGGAGACGTGGAGGAGAACCCTGGACCTATGGTGAGCAAG
GGCGAGGAGCTGTTCACCGGGGTGGTGCCCATCCTGGTCGAGCTGGACGGCGACGTAAACGGCC
ACAAGTTCAGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGACCTACGGC
GTGCAGTGCTTCAGCCGCTACCCCGACCACATGAAGCAGCACGACTTCTTCAAGTCCGCCATGC
CCGAAGGCTACGTCCAGGAGCGCACCATCTTCTTCAAGGACGACGGCAACTACAAGACCCGCGC
CGAGGTGAAGTTCGAGGGCGACACCCTGGTGAACCGCATCGAGCTGAAGGGCATCGACTTCAAG
GAGGACGGCAACATCCTGGGGCACAAGCTGGAGTACAACTACAACAGCCACAACGTCTATATCA
TGGCCGACAAGCAGAAGAACGGCATCAAGGTGAACTTCAAGATCCGCCACAACATCGAGGACGG
CAGCGTGCAGCTCGCCGACCACTACCAGCAGAACACCCCCATCGGCGACGGCCCCGTGCTGCTG
CCCGACAACCACTACCTGAGCACCCAGTCCGCCCTGAGCAAAGACCCCAACGAGAAGCGCGATC
ACATGGTCCTGCTGGAGTTCGTGACCGCCGCCGGGATCACTCTCGGCATGGACGAGCTGTACAA
GTGAGCGGCCGCGTCGAGTCTAGAGGGCCCGTTTAAACCCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGCAGAAATTTATGAAGCATTTGAAAACATCTACCCTATTCT
AAAGGGATTCAGGAAGACGACGTAATGGCTCTCATGTACCCTTGCCTCCCCCACCCCCTTCTTT
TTTTTTTTTTAAAGAAATCAGTTTGTTTTGGTACCTTTAAATGGTGGTGTTGTGAGAAGATGGA
TGTTGAGTTGCAGGGTGTGGCACCAGGTGATGCCCTTCTGTAAGTGCCCACCGCGGGATGCCGG
GAAGGGGCATTATTTGTGCACTGAGAACACCGCGCAGCGTGACTGTGAGTTGCTCATACCGTGC
TGCTATCTGGGCAGCGCTGCCCATTTATTTATATGTAGATTTTAAACACTGCTGTTGACAAGTT
GGTTTGAGGGAGAAAACTTTAAGTGTTAAAGCCACCTCTATAATTGATTGGACTTTTTAATTTT
AATGTTTTTCCCCATGAACCACAGTTTTTATATTTCTACCAGAAAAGTAAAAATCTTTTTTAAA
AGTGTTGTTTTT
exemplary donor template for insertion at TBP locus
SEQ ID NO: 49
CTGACCACAGCTCTGCAAGCAGACTTCCATTTACAGTGAGGAGGTGAGCATTGCATTGAACAAA
AGATGGCGTTTTCACTTGGAATTAGTTATCTGAAGCTTTAGGATTCCTCAGCAATATGATTATG
AGACAAGAAAGGAAGATTCAGAAATGAGTCTAGTTGAAGGCAGCAATTCAGAGAAGAAGATTCA
GTTGTTATCATTGCCGTCCTGCTTGGTTTATGGCCTGGTTCAGGACCAAGGAGAGAAGTGTGAA
TACATGCCTCTTGAGCTATAGAATGAGACGCTGGAGTCACTAAGATGATTTTTTAAAAGTATTG
TTTTATAAACAAAAATAAGATTGTGACAAGGGATTCCACTATTAATGTTTTCATGCCTGTGCCT
TAATCTGACTGGGTATGGTGAGAATTGTGCTTGCAGCTTTAAGGTAAGAATTTTACCATCTTAA
TATGTTAAGAAGTGCCATTTCAGTCTCTCATCTCTACTCCAACTTGTCTTCTTAGGGGCTAAAG
TGCGGGCCGAGATCTACGAGGCCTTCGAGAATATCTACCCCATCCTGAAGGGCTTCAGAAAGAC
CACCGGAAGCGGAGCTACTAACTTCAGCCTGCTGAAGCAGGCTGGAGACGTGGAGGAGAACCCT
GGACCTATGGTGAGCAAGGGCGAGGAGCTGTTCACCGGGGTGGTGCCCATCCTGGTCGAGCTGG
ACGGCGACGTAAACGGCCACAAGTTCAGCGTGTCCGGCGAGGGCGAGGGCGATGCCACCTACGG
CAAGCTGACCCTGAAGTTCATCTGCACCACCGGCAAGCTGCCCGTGCCCTGGCCCACCCTCGTG
ACCACCCTGACCTACGGCGTGCAGTGCTTCAGCCGCTACCCCGACCACATGAAGCAGCACGACT
TCTTCAAGTCCGCCATGCCCGAAGGCTACGTCCAGGAGCGCACCATCTTCTTCAAGGACGACGG
CAACTACAAGACCCGCGCCGAGGTGAAGTTCGAGGGCGACACCCTGGTGAACCGCATCGAGCTG
AAGGGCATCGACTTCAAGGAGGACGGCAACATCCTGGGGCACAAGCTGGAGTACAACTACAACA
GCCACAACGTCTATATCATGGCCGACAAGCAGAAGAACGGCATCAAGGTGAACTTCAAGATCCG
CCACAACATCGAGGACGGCAGCGTGCAGCTCGCCGACCACTACCAGCAGAACACCCCCATCGGC
GACGGCCCCGTGCTGCTGCCCGACAACCACTACCTGAGCACCCAGTCCGCCCTGAGCAAAGACC
CCAACGAGAAGCGCGATCACATGGTCCTGCTGGAGTTCGTGACCGCCGCCGGGATCACTCTCGG
CATGGACGAGCTGTACAAGTGAGCGGCCGCGTCGAGTCTAGAGGGCCCGTTTAAACCCGCTGAT
CAGCCTCGACTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCATCGCATTGT
CTGAGTAGGTGTCATTCTATTCTGGGGGGTGGGGTGGGGCAGGACAGCAAGGGGGAGGATTGGG
AAGACAATAGCAGGCATGCTGGGGATGCGGTGGGCTCTATGGTAGGTGCTAAAGTCAGAGCAGA
AATTTATGAAGCATTTGAAAACATCTACCCTATTCTAAAGGGATTCAGGAAGACGACGTAATGG
CTCTCATGTACCCTTGCCTCCCCCACCCCCTTCTTTTTTTTTTTTTAAACAAATCAGTTTGTTT
TGGTACCTTTAAATGGTGGTGTTGTGAGAAGATGGATGTTGAGTTGCAGGGTGTGGCACCAGGT
GATGCCCTTCTGTAAGTGCCCACCGCGGGATGCCGGGAAGGGGCATTATTTGTGCACTGAGAAC
ACCGCGCAGCGTGACTGTGAGTTGCTCATACCGTGCTGCTATCTGGGCAGCGCTGCCCATTTAT
TTATATGTAGATTTTAAACACTGCTGTTGAGAAGTTGGTTTGAGGGAGAAAACTTTAAGTGTTA
AAGCCACCTCTATAATTGATTGGACTTTTTAATTTTAATGTTTTTCCCCATGAACCACAGTTTT
TATATTTCTACCAGAAAAGTAAAAATCTTT
exemplary donor template for insertion at TBP locus
SEQ ID NO: 50
ACAAAAGATGGCGTTTTCACTTGGAATTAGTTATCTGAAGCTTTAGGATTCCTCAGCAATATGA
TTATGAGACAAGAAAGGAAGATTCAGAAATGAGTCTAGTTGAAGGCAGCAATTCAGAGAAGAAG
ATTCAGTTGTTATCATTGCCGTCCTGCTTGGTTTATGGCCTGGTTCAGGACCAAGGAGAGAAGT
GTGAATACATGCCTCTTGAGCTATAGAATGAGACGCTGGAGTCACTAAGATGATTTTTTAAAAG
TATTGTTTTATAAACAAAAATAAGATTGTGACAAGGGATTCCACTATTAATGTTTTCATGCCTG
TGCCTTAATCTGACTGGGTATGGTGAGAATTGTGCTTGCAGCTTTAAGGTAAGAATTTTACCAT
CTTAATATGTTAAGAAGTGCCATTTCAGTCTCTCATCTCTACTCCAACTTGTCTTCTTAGGTGC
TAAAGTCAGAGCAGAAATTTATGAAGCATTCGAGAACATCTACCCTATTCTAAAGGGATTCAGG
AAGACGACGGGAAGCGGAGCTACTAACTTCAGCCTGCTGAAGCAGGCTGGAGACGTGGAGGAGA
ACCCTGGACCTATGGTGAGCAAGGGCGAGGAGCTGTTCACCGGGGTGGTGCCCATCCTGGTCGA
GCTGGACGGCGACGTAAACGGCCACAAGTTCAGCGTGTCCGGCGAGGGCGAGGGCGATGCCACC
TACGGCAAGCTGACCCTGAAGTTCATCTGCACCACCGGCAAGCTGCCCGTGCCCTGGCCCACCC
TCGTGACCACCCTGACCTACGGCGTGCAGTGCTTCAGCCGCTACCCCGACCACATGAAGCAGCA
CGACTTCTTCAAGTCCGCCATGCCCGAAGGCTACGTCCAGGAGCGCACCATCTTCTTCAAGGAC
GACGGCAACTACAAGACCCGCGCCGAGGTGAAGTTCGAGGGCGACACCCTGGTGAACCGCATCG
AGCTGAAGGGCATCGACTTCAAGGAGGACGGCAACATCCTGGGGCACAAGCTGGAGTACAACTA
CAACAGCCACAACGTCTATATCATGGCCGACAAGCAGAAGAACGGCATCAAGGTGAACTTCAAG
ATCCGCCACAACATCGAGGACGGCAGCGTGCAGCTCGCCGACCACTACCAGCAGAACACCCCCA
TCGGCGACGGCCCCGTGCTGCTGCCCGACAACCACTACCTGAGCACCCAGTCCGCCCTGAGCAA
AGACCCCAACGAGAAGCGCGATCACATGGTCCTGCTGGAGTTCGTGACCGCCGCCGGGATCACT
CTCGGCATGGACGAGCTGTACAAGTGAGCGGCCGCGTCGAGTCTAGAGGGCCCGTTTAAACCCG
CTGATCAGCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCAAGGGGGAGGA
TTGGGAAGACAATAGCAGGCATGCTGGGGATGCGGTGGGCTCTATGGAAGGGATTCAGGAAGAC
GACGTAATGGCTCTCATGTACCCTTGCCTCCCCCACCCCCTTCTTTTTTTTTTTTTAAACAAAT
CAGTTTGTTTTGGTACCTTTAAATGGTGGTGTTGTGAGAAGATGGATGTTGAGTTGCAGGGTGT
GGCACCAGGTGATGCCCTTCTGTAAGTGCCCACCGCGGGATGCCGGGAAGGGGCATTATTTGTG
CACTGAGAACACCGCGCAGCGTGACTGTGAGTTGCTCATACCGTGCTGCTATCTGGGCAGCGCT
GCCCATTTATTTATATGTAGATTTTAAACACTGCTGTTGACAAGTTGGTTTGAGGGAGAAAACT
TTAAGTGTTAAAGCCACCTCTATAATTGATTGGACTTTTTAATTTTAATGTTTTTCCCCATGAA
CCACAGTTTTTATATTTCTACCAGAAAAGTAAAAATCTTTTTTAAAAGTGTTGTTTTTCTAATT
TATAACTCCTAGGGGTTATTTCTGTGCCAGACACA
exemplary donor template for insertion at G6PD locus
SEQ ID NO: 51
GGCCCGGGGGACTCCACATGGTGGCAGGCAGTGGCATCAGCAAGACACTCTCTCCCTCACAGAA
CGTGAAGCTCCCTGACGCCTATGAGCGCCTCATCCTGGACGTCTTCTGCGGGAGCCAGATGCAC
TTCGTGCGCAGGTGAGGCCCAGCTGCCGGCCCCTGCATACCTGTGGGCTATGGGGTGGCCTTTG
CCCTCCCTCCCTGTGTGCCACCGGCCTCCCAAGCCATACCATGTCCCCTCAGCGACGAGCTCCG
TGAGGCCTGGCGTATTTTCACCCCACTGCTGCACCAGATTGAGCTGGAGAAGCCCAAGCCCATC
CCCTATATTTATGGCAGGTGAGGAAAGGGTGGGGGCTGGGGACAGAGCCCAGCGGGCAGGGGCG
GGGTGAGGGTGGAGCTACCTCATGCCTCTCCTCCACCCGTCACTCTCCAGCCGAGGCCCCACGG
AGGCAGACGAGCTGATGAAGAGAGTGGGCTTCCAGTACGAGGGAACCTACAAATGGGTCAACCC
TCACAAGCTGGGAAGCGGAGCTACTAACTTCAGCCTGCTGAAGCAGGCTGGAGACGTGGAGGAG
AACCCTGGACCTATGGTGAGCAAGGGCGAGGAGCTGTTCACCGGGGTGGTGCCCATCCTGGTCG
AGCTGGACGGCGACGTAAACGGCCACAAGTTCAGCGTGTCCGGCGAGGGCGAGGGCGATGCCAC
CTACGGCAAGCTGACCCTGAAGTTCATCTGCACCACCGGCAAGCTGCCCGTGCCCTGGCCCACC
CTCGTGACCACCCTGACCTACGGCGTGCAGTGCTTCAGCCGCTACCCCGACCACATGAAGCAGC
ACGACTTCTTCAAGTCCGCCATGCCCGAAGGCTACGTCCAGGAGCGCACCATCTTCTTCAAGGA
CGACGGCAACTACAAGACCCGCGCCGAGGTGAAGTTCGAGGGCGACACCCTGGTGAACCGCATC
GAGCTGAAGGGCATCGACTTCAAGGAGGACGGCAACATCCTGGGGCACAAGCTGGAGTACAACT
ACAACAGCCACAACGTCTATATCATGGCCGACAAGCAGAAGAACGGCATCAAGGTGAACTTCAA
GATCCGCCACAACATCGAGGACGGCAGCGTGCAGCTCGCCGACCACTACCAGCAGAACACCCCC
ATCGGCGACGGCCCCGTGCTGCTGCCCGACAACCACTACCTGAGCACCCAGTCCGCCCTGAGCA
AAGACCCCAACGAGAAGCGCGATCACATGGTCCTGCTGGAGTTCGTGACCGCCGCCGGGATCAC
TCTCGGCATGGACGAGCTGTACAAGTGAGCGGCCGCGTCGAGTCTAGAGGGCCCGTTTAAACCC
GCTGATCAGCCTCGACTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCAAGGGGGAGG
ATTGGGAAGACAATAGCAGGCATGCTGGGGATGCGGTGGGCTCTATGGGTGGGTGAACCCCCAC
AAGCTCTGAGCCCTGGGCACCCACCTCCACCCCCGCCACGGCCACCCTCCTTCCCGCCGCCCGA
CCCCGAGTCGGGAGGACTCCGGGACCATTGACCTCAGCTGCACATTCCTGGCCCCGGGCTCTGG
CCACCCTGGCCCGCCCCTCGCTGCTGCTACTACCCGAGCCCAGCTACATTCCTCAGCTGCCAAG
CACTCGAGACCATCCTGGCCCCTCCAGACCCTGCCTGAGCCCAGGAGCTGAGTCACCTCCTCCA
CTCACTCCAGCCCAACAGAAGGAAGGAGGAGGGCGCCCATTCGTCTGTCCCAGAGCTTATTGGC
CACTGGGTCTCACTCCTGAGTGGGGCCAGGGTGGGAGGGAGGGACGAGGGGGAGGAAAGGGGCG
AGCACCCACGTGAGAGAATCTGCCTGTGGCCTTGCCCGCCAGCCTCAGTGCCACTTGACATTCC
TTGTCACCAGCAACATCTCGAGCCCCCTGGATGTCC
exemplary donor template for insertion at E2F4 locus
SEQ ID NO: 52
CCAGGGGGCTGTAGTGGGGCCAGGCTGGACCTCTGTGCCCTGAGCATGGCTTTCTTGTTTTTCA
GTTTTGGAACTCCCCAAAGAGCTGTCAGAAATCTTTGATCCCACACGAGGTAGGCTGCTGCATT
CCTCCCTGAGGCTAGGGGTAAGGGACACAGCTCATTGGGTCCTATGGCTGTTTTCTTGCCCTTT
TGAGGACCTTGTTGTGGCGCTTATGGTAACTGGGGCAAAGGGTGAAGTTCCTGATGGGCAGGTG
GGGTTCCCTTTCCTGGGCTTTGGTGGGTGGAGAGGTGGGAGCTGGAATGTTAGTAACTGAGCTC
CCTCCATTCCCAGAGTGCATGAGCTCGGAGCTGCTGGAGGAGTTGATGTCCTCAGAAGGTGGGT
GGCCCTGGAAGGTGGGAGTGGGTGTGGGCAGGGGTTGGGCTGCTGCTAGGGGAGCCCTGGCCCA
GGGCCTGAGACTAGTGCTCTCTGCAGTGTTCGCCCCTCTGCTGAGACTTTCTCCTCCTCCTGGC
GACCACGACTACATCTACAACCTGGACGAGAGCGAGGGCGTGTGCGACCTGTTTGATGTGCCCG
TGCTGAACCTGGGAAGCGGAGCTACTAACTTCAGCCTGCTGAAGCAGGCTGGAGACGTGGAGGA
GAACCCTGGACCTATGGTGAGCAAGGGCGAGGAGCTGTTCACCGGGGTGGTGCCCATCCTGGTC
GAGCTGGACGGCGACGTAAACGGCCACAAGTTCAGCGTGTCCGGCGAGGGCGAGGGCGATGCCA
CCTACGGCAAGCTGACCCTGAAGTTCATCTGCACCACCGGCAAGCTGCCCGTGCCCTGGCCCAC
CCTCGTGACCACCCTGACCTACGGCGTGCAGTGCTTCAGCCGCTACCCCGACCACATGAAGCAG
CACGACTTCTTCAAGTCCGCCATGCCCGAAGGCTACGTCCAGGAGCGCACCATCTTCTTCAAGG
ACGACGGCAACTACAAGACCCGCGCCGAGGTGAAGTTCGAGGGCGACACCCTGGTGAACCGCAT
CGAGCTGAAGGGCATCGACTTCAAGGAGGACGGCAACATCCTGGGGCACAAGCTGGAGTACAAC
TACAACAGCCACAACGTCTATATCATGGCCGACAAGCAGAAGAACGGCATCAAGGTGAACTTCA
AGATCCGCCACAACATCGAGGACGGCAGCGTGCAGCTCGCCGACCACTACCAGCAGAACACCCC
CATCGGCGACGGCCCCGTGCTGCTGCCCGACAACCACTACCTGAGCACCCAGTCCGCCCTGAGC
AAAGACCCCAACGAGAAGCGCGATCACATGGTCCTGCTGGAGTTCGTGACCGCCGCCGGGATCA
CTCTCGGCATGGACGAGCTGTACAAGTGAGCGGCCGCGTCGAGTCTAGAGGGCCCGTTTAAACC
CGCTGATCAGCCTCGACTGTGCCTTCTAGTTGCCAGCCATCTGTTGTTTGCCCCTCCCCCGTGC
CTTCCTTGACCCTGGAAGGTGCCACTCCCACTGTCCTTTCCTAATAAAATGAGGAAATTGCATC
GCATTGTCTGAGTAGGTGTCATTCTATTCTGGGGGGTGGGGTGGGGCAGGACAGCAAGGGGGAG
GATTGGGAAGACAATAGCAGGCATGCTGGGGATGCGGTGGGCTCTATGGCCACCCCCGGGAGAC
CACGATTATATCTACAACCTGGACGAGAGTGAAGGTGTCTGTGACCTCTTTGATGTGCCTGTTC
TCAACCTCTGACTGACAGGGACATGCCCTGTGTGGCTGGGACCCAGACTGTCTGACCTGGGGGT
TGCCTGGGGACCTCTCCCACCCGACCCCTACAGAGCTTGAGAGCCACAGACGCCTGGCTTCTCC
GGCCTCCCCTCACCGCACAGTTCTGGCCACAGCTCCCGCTCCTGTGCTGGCACTTCTGTGCTCG
CAGAGCAGGGGAACAGGACTCAGCCCCCATCACCGTGGAGCCAAAGTGTTTGCTTCTCCCTTTC
TGCGGCCTTCGCCAGCCCAGGCTCGGCTGCCACCCAGTGGCACAGAACCGAGGAGCTGCCATTA
CCCCCCATAGGGGGCAGTGTCTTGTTCCTGCCAGCCTCAGTGTCTTGCTTCTGCCAGCTCCTTC
CCCTAGGAGGGAAGGGTGGGGTGGAACTGGGCACATG
exemplary donor template for insertion at E2F4 locus
SEQ ID NO: 53
CCAGGCTGGACCTCTGTGCCCTGAGCATGGCTTTCTTGTTTTTCAGTTTTGGAACTCCCCAAAG
AGCTGTCAGAAATCTTTGATCCCACACGAGGTAGGCTGCTGCATTCCTCCCTGAGGCTAGGGGT
AAGGGACACAGCTCATTGGGTCCTATGGCTGTTTTCTTGCCCTTTTGAGGACCTTGTTGTGGCG
CTTATGGTAACTGGGGCAAAGGGTGAAGTTCCTGATGGGCAGGTGGGGTTCCCTTTCCTGGGCT
TTGGTGGGTGGAGAGGTGGGAGCTGGAATGTTAGTAACTGAGCTCCCTCCATTCCCAGAGTGCA
TGAGCTCGGAGCTGCTGGAGGAGTTGATGTCCTCAGAAGGTGGGTGGCCCTGGAAGGTGGGAGT
GGGTGTGGGCAGGGGTTGGGCTGCTGCTAGGGGAGCCCTGGCCCAGGGCCTGAGACTAGTGCTC
TCTGCAGTGTTTGCCCCTCTGCTTCGTCTTAGTCCTCCTCCGGGCGACCACGACTACATCTACA
ACCTGGACGAGAGCGAGGGCGTGTGCGACCTGTTTGATGTGCCCGTGCTGAACCTGGGAAGCGG
AGCTACTAACTTCAGCCTGCTGAAGCAGGCTGGAGACGTGGAGGAGAACCCTGGACCTATGGTG
AGCAAGGGCGAGGAGCTGTTCACCGGGGTGGTGCCCATCCTGGTCGAGCTGGACGGCGACGTAA
ACGGCCACAAGTTCAGCGTGTCCGGCGAGGGCGAGGGCGATGCCACCTACGGCAAGCTGACCCT
GAAGTTCATCTGCACCACCGGCAAGCTGCCCGTGCCCTGGCCCACCCTCGTGACCACCCTGACC
TACGGCGTGCAGTGCTTCAGCCGCTACCCCGACCACATGAAGCAGCACGACTTCTTCAAGTCCG
CCATGCCCGAAGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAAGGGCATCGAC
TTCAAGGAGGACGGCAACATCCTGGGGCACAAGCTGGAGTACAACTACAACAGCCACAACGTCT
ATATCATGGCCGACAAGCAGAAGAACGGCATCAAGGTGAACTTCAAGATCCGCCACAACATCGA
GGACGGCAGCGTGCAGCTCGCCGACCACTACCAGCAGAACACCCCCATCGGCGACGGCCCCGTG
CTGCTGCCCGACAACCACTACCTGAGCACCCAGTCCGCCCTGAGCAAAGACCCCAACGAGAAGC
GCGATCACATGGTCCTGCTGGAGTTCGTGACCGCCGCCGGGATCACTCTCGGCATGGACGAGCT
GTACAAGTGAGCGGCCGCGTCGAGTCTAGAGGGCCCGTTTAAACCCGCTGATCAGCCTCGACTG
TGCCTTCTAGTTGCCAGCCATCTGTTGTTTGCCCCTCCCCCGTGCCTTCCTTGACCCTGGAAGG
TGCCACTCCCACTGTCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTGGGAAGACAATAGCA
GGCATGCTGGGGATGCGGTGGGCTCTATGGATTATATCTACAACCTGGACGAGAGTGAAGGTGT
CTGTGACCTCTTTGATGTGCCTGTTCTCAACCTCTGACTGACAGGGACATGCCCTGTGTGGCTG
GGACCCAGACTGTCTGACCTGGGGGTTGCCTGGGGACCTCTCCCACCCGACCCCTACAGAGCTT
GAGAGCCACAGACGCCTGGCTTCTCCGGCCTCCCCTCACCGCACAGTTCTGGCCACAGCTCCCG
CTCCTGTGCTGGCACTTCTGTGCTCGCAGAGCAGGGGAACAGGACTCAGCCCCCATCACCGTGG
AGCCAAAGTGTTTGCTTCTCCCTTTCTGCGGCCTTCGCCAGCCCAGGCTCGGCTGCCACCCAGT
GGCACAGAACCGAGGAGCTGCCATTACCCCCCATAGGGGGCAGTGTCTTGTTCCTGCCAGCCTC
AGTGTCTTGCTTCTGCCAGCTCCTTCCCCTAGGAGGGAAGGGTGGGGTGGAACTGGGCACATGC
CAGCACCACTTGTAGCTT
exemplary donor template for insertion at E2F4 locus
SEQ ID NO: 54
GTCAGAAATCTTTGATCCCACACGAGGTAGGCTGCTGCATTCCTCCCTGAGGCTAGGGGTAAGG
GACACAGCTCATTGGGTCCTATGGCTGTTTTCTTGCCCTTTTGAGGACCTTGTTGTGGCGCTTA
TGGTAACTGGGGCAAAGGGTGAAGTTCCTGATGGGCAGGTGGGGTTCCCTTTCCTGGGCTTTGG
TGGGTGGAGAGGTGGGAGCTGGAATGTTAGTAACTGAGCTCCCTCCATTCCCAGAGTGCATGAG
CTCGGAGCTGCTGGAGGAGTTGATGTCCTCAGAAGGTGGGTGGCCCTGGAAGGTGGGAGTGGGT
GTGGGCAGGGGTTGGGCTGCTGCTAGGGGAGCCCTGGCCCAGGGCCTGAGACTAGTGCTCTCTG
CAGTGTTTGCCCCTCTGCTTCGTCTTTCTCCACCCCCGGGAGACCACGATTATATCTACAACCT
GGACGAGAGTGAAGGTGTCTGTGACCTCTTCGACGTGCCCGTGCTCAACCTCGGAAGCGGAGCT
ACTAACTTCAGCCTGCTGAAGCAGGCTGGAGACGTGGAGGAGAACCCTGGACCTATGGTGAGCA
AGGGCGAGGAGCTGTTCACCGGGGTGGTGCCCATCCTGGTCGAGCTGGACGGCGACGTAAACGG
CCACAAGTTCAGCGTGTCCGGCGAGGGCGAGGGCGATGCCACCTACGGCAAGCTGACCCTGAAG
TTCATCTGCACCACCGGCAAGCTGCCCGTGCCCTGGCCCACCCTCGTGACCACCCTGACCTACG
GCGTGCAGTGCTTCAGCCGCTACCCCGACCACATGAAGCAGCACGACTTCTTCAAGTCCGCCAT
GCCCGAAGGCTACGTCCAGGAGCGCACCATCTTCTTCAAGGACGACGGCAACTACAAGACCCGC
GCCGAGGTGAAGTTCGAGGGCGACACCCTGGTGAACCGCATCGAGCTGAAGGGCATCGACTTCA
AGGAGGACGGCAACATCCTGGGGCACAAGCTGGAGTACAACTACAACAGCCACAACGTCTATAT
CATGGCCGACAAGCAGAAGAACGGCATCAAGGTGAACTTCAAGATCCGCCACAACATCGAGGAC
GGCAGCGTGCAGCTCGCCGACCACTACCAGCAGAACACCCCCATCGGCGACGGCCCCGTGCTGC
TGCCCGACAACCACTACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCATGGACGAGCTGTAC
AAGTGAGCGGCCGCGTCGAGTCTAGAGGGCCCGTTTAAACCCGCTGATCAGCCTCGACTGTGCC
TTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATT
CTATTCTGGGGGGTGGGGTGGGGCAGGACAGCAAGGGGGAGGATTGGGAAGACAATAGCAGGCA
TGCTGGGGATGCGGTGGGCTCTATGGTGACTGACAGGGACATGCCCTGTGTGGCTGGGACCCAG
ACTGTCTGACCTGGGGGTTGCCTGGGGACCTCTCCCACCCGACCCCTACAGAGCTTGAGAGCCA
CAGACGCCTGGCTTCTCCGGCCTCCCCTCACCGCACAGTTCTGGCCACAGCTCCCGCTCCTGTG
CTGGCACTTCTGTGCTCGCAGAGCAGGGGAACAGGACTCAGCCCCCATCACCGTGGAGCCAAAG
TGTTTGCTTCTCCCTTTCTGCGGCCTTCGCCAGCCCAGGCTCGGCTGCCACCCAGTGGCACAGA
ACCGAGGAGCTGCCATTACCCCCCATAGGGGGCAGTGTCTTGTTCCTGCCAGCCTCAGTGTCTT
GCTTCTGCCAGCTCCTTCCCCTAGGAGGGAAGGGTGGGGTGGAACTGGGCACATGCCAGCACCA
CTTCTAGCTTCCTTCGCTATCCCCCACCCCCTGACCCTCCAGCTCCTCCTGGCCCTCTCACGTG
CCCACTTCTGCTGG
exemplary donor template for insertion at KIF11 locus
SEQ ID NO: 55
AGAGCAGGGTTTCTTGACAGCAGTGCTATTGGCATTTTAAACTGGATAATTCTTTGTTGTGATG
GGCTTTCCTGTGGAGTGTACTATGTTGGTACACAAGAAAAACAGTGTACTATGTGAATACTCAC
TCAAAGCCAGTAGCACTCCCTGATTGTAACACCAAAAAAGTCTCTCAGCATTGCCAAATGTCCC
CTGTGGCAGCAGAATCACTCCCTGATGAGAACCACTACCCTGGAGTAAAATCTATAACTATGTC
TTAGAAAATAACACAGAAAATTAATATTTCTTTCACTCTACTCCTTCCATTAGTGATCAAATAA
AGAAGGCATTTGGCGCTACTTGCCAAATTGTTGGCTCAAACTTGTGCTGAACCTTTTTTGGTTT
TCTACACTTAAGTTTTTTTGCCTATAACCCAGAGAACTTTGAAAATAGAGTGTAGTTAATGTGT
ATCTAATGTTACTTTGTATTGACTTAATTTACCGGCCTTTAATCCACAGCATAAGAAGTCCCAC
GGCAAGGACAAAGAGAACCGGGGCATCAACACACTGGAACGGTCCAAGGTCGAGGAAACAACCG
AGCACCTGGTCACCAAGAGCAGACTGCCTCTGAGAGCCCAGATCAACCTGGGAAGCGGAGCTAC
TAACTTCAGCCTGCTGAAGCAGGCTGGAGACGTGGAGGAGAACCCTGGACCTATGGTGAGCAAG
GGCGAGGAGCTGTTCACCGGGGTGGTGCCCATCCTGGTCGAGCTGGACGGCGACGTAAACGGCC
ACAAGTTCAGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGACCTACGGC
GTGCAGTGCTTCAGCCGCTACCCCGACCACATGAAGCAGCACGACTTCTTCAAGTCCGCCATGC
CCGAAGGCTACGTCCAGGAGCGCACCATCTTCTTCAAGGACGACGGCAACTACAAGACCCGCGC
CGAGGTGAAGTTCGAGGGCGACACCCTGGTGAACCGCATCGAGCTGAAGGGCATCGACTTCAAG
GAGGACGGCAACATCCTGGGGCACAAGCTGGAGTACAACTACAACAGCCACAACGTCTATATCA
TGGCCGACAAGCAGAAGAACGGCATCAAGGTGAACTTCAAGATCCGCCACAACATCGAGGACGG
CAGCGTGCAGCTCGCCGACCACTACCAGCAGAACACCCCCATCGGCGACGGCCCCGTGCTGCTG
CCCGACAACCACTACCTGAGCACCCAGTCCGCCCTGAGCAAAGACCCCAACGAGAAGCGCGATC
ACATGGTCCTGCTGGAGTTCGTGACCGCCGCCGGGATCACTCTCGGCATGGACGAGCTGTACAA
GTGAGCGGCCGCGTCGAGTCTAGAGGGCCCGTTTAAACCCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAAAAATCACATGGAAAAGACAAAGAAAACAGAGGCATTA
ACACACTGGAGAGGTCTAAAGTGGAAGAAACTACAGAGCACTTGGTTACAAAGAGCAGATTACC
TCTGCGAGCCCAGATCAACCTTTAATTCACTTGGGGGTTGGCAATTTTATTTTTAAAGAAAACT
TAAAAATAAAACCTGAAACCCCAGAACTTGAGCCTTGTGTATAGATTTTAAAAGAATATATATA
TCAGCCGGGCGCGGTGGCTCATGCCTGTAATCCCAGCACTTTGGGAGGCTGAGGCGGGTGGATT
GCTTGAGCCCAGGAGTTTGAGACCAGCCTGGCCAACGTGGCAAAACCTCGTCTCTGTTAAAAAT
TAGCCGGGCGTGGTGGCACACTCCTGTAATCCCAGCTACTGGGGAGGCTGAGGCACGAGAATCA
CTTGAACCCAGGAAGCGGGGTTGCAGTGAGCCAAAGGTACACCACTACACTCCAGCCTGGGCAA
CAGAGCAAGACT
exemplary donor template for insertion at KFF11 locus
SEQ ID NO: 56
TTCCTGTGGACTGTACTATGTTGGTACACAAGAAAAACAGTGTACTATGTGAATACTCACTCAA
AGCCAGTAGCACTCCCTGATTGTAACACCAAAAAAGTCTCTCAGCATTGCCAAATGTCCCCTGT
GGCAGCAGAATCACTCCCTGATGAGAACCACTACCCTGGAGTAAAATCTATAACTATGTCTTAG
AAAATAACACAGAAAATTAATATTTCTTTCACTCTACTCCTTCCATTAGTGATCAAATAAAGAA
GGCATTTGGCGCTACTTGCCAAATTGTTGGCTCAAACTTGTGCTGAACCTTTTTTGGTTTTCTA
CACTTAAGTTTTTTTGCCTATAACCCAGAGAACTTTGAAAATAGAGTGTAGTTAATGTGTATCT
AATGTTACTTTGTATTGACTTAATTTTCCCGCCTTAAATCCACAGCATAAAAAATCACATGGAA
AAGACAAAGAAAACAGAGGCATTAACACACTGGAGAGGTCTAAAGTGGAAGAAACAACCGAGCA
CCTGGTCACCAAGAGCAGACTGCCTCTGAGAGCCCAGATCAACCTGGGAAGCGGAGCTACTAAC
TTCAGCCTGCTGAAGCAGGCTGGAGACGTGGAGGAGAACCCTGGACCTATGGTGAGCAAGGGCG
AGGAGCTGTTCACCGGGGTGGTGCCCATCCTGGTCGAGCTGGACGGCGACGTAAACGGCCACAA
GTTCAGCGTGTCCGGCGAGGGCGAGGGCGATGCCACCTACGGCAAGCTGACCCTGAAGTTCATC
TGCACCACCGGCAAGCTGCCCGTGCCCTGGCCCACCCTCGTGACCACCCTGACCTACGGCGTGC
AGTGCTTCAGCCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGACCCGCGCCGAG
GTGAAGTTCGAGGGCGACACCCTGGTGAACCGCATCGAGCTGAAGGGCATCGACTTCAAGGAGG
ACGGCAACATCCTGGGGCACAAGCTGGAGTACAACTACAACAGCCACAACGTCTATATCATGGC
CGACAAGCAGAAGAACGGCATCAAGGTGAACTTCAAGATCCGCCACAACATCGAGGACGGCAGC
GTGCAGCTCGCCGACCACTACCAGCAGAACACCCCCATCGGCGACGGCCCCGTGCTGCTGCCCG
ACAACCACTACCTGAGCACCCAGTCCGCCCTGAGCAAAGACCCCAACGAGAAGCGCGATCACAT
GGTCCTGCTGGAGTTCGTGACCGCCGCCGGGATCACTCTCGGCATGGACGAGCTGTACAAGTGA
GCGGCCGCGTCGAGTCTAGAGGGCCCGTTTAAACCCGCTGATCAGCCTCGACTGTGCCTTCTAG
TTGCCAGCCATCTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTGTCATTCTATTC
TGGGGGGTGGGGTGGGGCAGGACAGCAAGGGGGAGGATTGGGAAGACAATAGCAGGCATGCTGG
GGATGCGGTGGGCTCTATGGAACTACAGAGCACTTGGCTACATAGAGCAGATTACCTCTGCGAG
CCCAGATCAACCTTTAATTCACTTGGGGGTTGGCAATTTTATTTTTAAAGAAAACTTAAAAATA
AAACCTGAAACCCCAGAACTTGAGCCTTGTGTATAGATTTTAAAAGAATATATATATCAGCCGG
GCGCGGTGGCTCATGCCTGTAATCCCAGCACTTTGGGAGGCTGAGGCGGGTGGATTGCTTGAGC
CCAGGAGTTTGAGACCAGCCTGGCCAACGTGGCAAAACCTCGTCTCTGTTAAAAATTAGCCGGG
CGTGGTGGCACACTCCTGTAATCCCAGCTACTGGGGAGGCTGAGGCACGAGAATCACTTGAACC
CAGGAAGCGGGGTTGCAGTGAGCCAAAGGTACACCACTACACTCCAGCCTGGGCAACAGAGCAA
GACTCGGTCTCAAAAACAAAATTTAAAAAAGATATAAGGCAGTACTGTAAATTCAGTTGAATTT
TGATATCT
exemplary donor template for insertion at at KFF11 locus
SEQ ID NO: 57
TTAAACTGGATAATTCTTTGTTGTGATGGGCTTTCCTGTGGACTGTACTATGTTGGTACACAAG
AAAAACAGTGTACTATGTGAATACTCACTCAAAGCCAGTAGCACTCCCTGATTGTAACACCAAA
AAAGTCTCTCAGCATTGCCAAATGTCCCCTGTGGCAGCAGAATCACTCCCTGATGAGAACCACT
ACCCTGGAGTAAAATCTATAACTATGTCTTAGAAAATAACACAGAAAATTAATATTTCTTTCAC
TCTACTCCTTCCATTAGTGATCAAATAAAGAAGGCATTTGGCGCTACTTGCCAAATTGTTGGCT
CAAACTTGTGCTGAACCTTTTTTGGTTTTCTACACTTAAGTTTTTTTGCCTATAACCCAGAGAA
CTTTGAAAATAGAGTGTAGTTAATGTGTATCTAATGTTACTTTGTATTGACTTAATTTTCCCGC
CTTAAATCCACAGCATAAAAAATCACATGGAAAAGACAAAGAAAACAGAGGCATCAACACACTG
GAACGGTCCAAGGTCGAGGAAACAACCGAGCACCTGGTCACCAAGAGCAGACTGCCTCTGAGAG
CCCAGATCAACCTGGGAAGCGGAGCTACTAACTTCAGCCTGCTGAAGCAGGCTGGAGACGTGGA
GGAGAACCCTGGACCTATGGTGAGCAAGGGCGAGGAGCTGTTCACCGGGGTGGTGCCCATCCTG
GTCGAGCTGGACGGCGACGTAAACGGCCACAAGTTCAGCGTGTCCGGCGAGGGCGAGGGCGATG
CCACCTACGGCAAGCTGACCCTGAAGTTCATCTGCACCACCGGCAAGCTGCCCGTGCCCTGGCC
CACCCTCGTGACCACCCTGACCTACGGCGTGCAGTGCTTCAGCCGCTACCCCGACCACATGAAG
CAGCACGACTTCTTCAAGTCCGCCATGCCCGAAGGCTACGTCCAGGAGCGCACCATCTTCTTCA
AGGACGACGGCAACTACAAGACCCGCGCCGAGGTGAAGTTCGAGGGCGACACCCTGGTGAACCG
CATCGAGCTGAAGGGCATCGACTTCAAGGAGGACGGCAACATCCTGGGGCACAAGCTGGAGTAC
AACTACAACAGCCACAACGTCTATATCATGGCCGACAAGCAGAAGAACGGCATCAAGGTGAACT
TCAAGATCCGCCACAACATCGAGGACGGCAGCGTGCAGCTCGCCGACCACTACCAGCAGAACAC
CCCCATCGGCGACGGCCCCGTGCTGCTGCCCGACAACCACTACCTGAGCACCCAGTCCGCCCTG
AGCAAAGACCCCAACGAGAAGCGCGATCACATGGTCCTGCTGGAGTTCGTGACCGCCGCCGGGA
TCACTCTCGGCATGGACGAGCTGTACAAGTGAGCGGCCGCGTCGAGTCTAGAGGGCCCGTTTAA
ACCCGCTGATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAGCAAGGGG
GAGGATTGGGAAGACAATAGCAGGCATGCTGGGGATGCGGTGGGCTCTATGGATTAACACACTG
GAGAGTTCTGAAGTGGAAGAAACTACAGAGCACTTGGTTACAAAGAGCAGATTACCTCTGCGAG
CCCAGATCAACCTTTAATTGAGTTGGGGGTTGGCAATTTTATTTTTAAAGAAAACTTAAAAATA
AAACCTGAAACCCCAGAACTTGAGCCTTGTGTATAGATTTTAAAAGAATATATATATGAGCCGG
GCGCGGTGGCTCATGCCTGTAATCCCAGCACTTTGGGAGGCTGAGGCGGGTGGATTGCTTGAGC
CCAGGAGTTTGAGACCAGCCTGGCCAACGTGGCAAAACCTCGTCTCTGTTAAAAATTAGCCGGG
CGTGGTGGCACACTCCTGTAATCCCAGCTACTGGGGAGGCTGAGGCACGAGAATCACTTGAACC
CAGGAAGCGGGGTTGCAGTGAGCCAAAGGTACACCACTACACTCCAGCCTGGGCAACAGAGCAA
GACTCGGTCTCAAAAACAAAATTTAAAAAAGATATAAGGC
exemplary donor template for insertion at GAPDH locus
SEQ ID NO: 48
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTGGCCCCTGGTAGCGG
CGCTGTTGCTGGGCTCGGCGTGCTGCGGATCAGCTCAGCTACTATTTAATAAAACAAAATCTGT
AGAATTCACGTTTTGTAATGACACTGTCGTCATTCCATGCTTTGTTACTAATATGGAGGCACAA
AACACTACTGAAGTATACGTAAAGTGGAAATTTAAAGGAAGAGATATTTACACCTTTGATGGAG
CTCTAAACAAGTCCACTGTCCCCACTGACTTTAGTAGTGCAAAAATTGAAGTCTCACAATTACT
AAAAGGAGATGCCTCTTTGAAGATGGATAAGAGTGATGCTGTCTCACACACAGGAAACTACACT
TGTGAAGTAACAGAATTAACCAGAGAAGGTGAAACGATCATCGAGCTAAAATATCGTGTTGTTT
CATGGTTTTCTCCAAATGAAAATATTCTTATTGTTATTTTCCCAATTTTTGCTATACTCCTGTT
CTGGGGACAGTTTGGTATTAAAACACTTAAATATAGATCCGGTGGTATGGATGAGAAAACAATT
GCTTTACTTGTTGCTGGACTAGTGATCACTGTCATTGTCATTGTTGGAGCCATTCTTTTCGTCC
CAGGTGAATATTCATTAAAGAATGCTACTGGCCTTGGTTTAATTGTGACTTCTACAGGGATATT
AATATTACTTCACTACTATGTGTTTAGTACAGCGATTGGATTAACCTCCTTCGTCATTGCCATA
TTGGTTATTCAGGTGATAGCCTATATCCTCGCTGTGGTTGGACTGAGTCTCTGTATTGCGGCGT
GTATACCAATGCATGGCCCTCTTCTGATTTCAGGTTTGAGTATCTTAGCTCTAGCACAATTACT
TGGACTAGTTTATATGAAATTTGTGGCTTCCAATCAGAAGACTATACAACCTCCTAGGAAAGCT
GTAGAGGAACCCCTTAATGCATTCAAAGAATCAAAAGGAATGATGAATGATGAATGAGCGGCCG
CGTCGAGTCTAGAGGGCCCGTTTAAACCCGCTGATCAGCCTCGACTGTGCCTTCTAGTTGCCAG
CCATCTGTTGTTTGCCCCTCCCCCGTGCCTTCCTTGACCCTGGAAGGTGCCACTCCCACTGTCC
TTTCCTAATAAAATGAGGAAATTGCATCGCATTGTCTGAGTAGGTGTCATTCTATTCTGGGGGG
TGGGGTGGGGCAGGACAGCAAGGGGGAGGATTGGGAAGACAATAGCAGGCATGCTGGGGATGCG
GTGGGCTCTATGGATTTGGCTACAGCAACAGGGTGGTGGACCTCATGGCCCACATGGCCTCCAA
GGAGTAAGACCCCTGGACCACCAGCCCCAGCAAGAGCACAAGAGGAAGAGAGAGACCCTCACTG
CTGGGGAGTCCCTGCCACACTCAGTCCCCCACCACACTGAATCTCCCCTCCTCACAGTTGCCAT
GTAGACCCCTTGAAGAGGGGAGGGGCCTAGGGAGCCGCACCTTGTCATGTACCATCAATAAAGT
ACCCTGTGCTCAACCAGTTACTTGTCCTGTCTTATTCTAGGGTCTGGGGCAGAGGGGAGGGAAG
CTGGGCTTGTGTCAAGGTGAGACATTCTTGCTGGGGAGGGACCTGGTATGTTCTCCTCAGACTG
AGGGTAGGGCCTCCAAACAGCCTTGCTTGCTTCGAGAACCATTTGCTTCCCGCTCAGACGTCTT
GAGTGCTACAGGAAGCTGGCACCACTACTTCAGAGAACAAGGCCTTTTCCTCTCCTCGCTCCAG
T
exemplary donor template for insertion at GAPDH locus
SEQ ID NO: 205
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTGGCAACTGCTGCTGC
CTACAGCTCTGCTGCTTCTGGTGTCTGCCGGCATGAGAACCGAGGATCTGCCTAAGGCCGTGGT
GTTCCTGGAACCTCAGTGGTACAGAGTGCTGGAAAAGGACAGCGTGACCCTGAAGTGCCAGGGC
GCCTATTCTCCCGAGGACAATAGCACCCAGTGGTTCCACAACGAGAGCCTGATCAGCAGCCAGG
CCAGCAGCTACTTTATCGATGCCGCCACCGTGGACGACAGCGGCGAGTACAGATGCCAGACCAA
TCTGAGCACCCTGAGCGACCCTGTGCAGCTGGAAGTGCACATTGGATGGTTGCTGCTGCAAGCC
CCTAGATGGGTGTTCAAAGAAGAGGACCCCATCCACCTGAGATGCCACTCTTGGAAGAACACAG
CCCTGCACAAAGTGACCTACCTGCAGAACGGCAAGGGCAGAAAGTACTTCCACCACAACAGCGA
CTTCTACATCCCCAAGGCCACACTGAAGGACTCCGGCTCCTACTTCTGCAGAGGCCTGGTCGGC
AGCAAGAACGTGTCCAGCGAGACAGTGAACATCACCATCACACAGGGCCTCGCCGTGTCTACCA
TCAGCAGCTTTTTCCCACCTGGCTATCAGGTGTCCTTCTGCCTGGTCATGGTGCTGCTGTTCGC
CGTGGATACCGGCCTGTACTTCAGCGTCAAGACCAACATCCGGTCCAGCACCAGAGACTGGAAG
GACCACAAGTTCAAGTGGCGGAAGGACCCTCAGGACAAGTAAGCGGCCGCGTCGAGTCTAGAGG
GCCCGTTTAAACCCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAT
TTGGCTACAGCAACAGGGTGGTGGACCTCATGGCCCACATGGCCTCCAAGGAGTAAGACCCCTG
GACCACCAGCCCCAGCAAGAGCACAAGAGGAAGAGAGAGACCCTCACTGCTGGGGAGTCCCTGC
CACACTCAGTCCCCCACCACACTGAATCTCCCCTCCTCACAGTTGCCATGTAGACCCCTTGAAG
AGGGGAGGGGCCTAGGGAGCCGCACCTTGTCATGTACCATCAATAAAGTACCCTGTGCTCAACC
AGTTACTTGTCCTGTCTTATTCTAGGGTCTGGGGCAGAGGGGAGGGAAGCTGGGCTTGTGTCAA
GGTGAGACATTCTTGCTGGGGAGGGACCTGGTATGTTCTCCTCAGACTGAGGGTAGGGCCTCCA
AACAGCCTTGCTTGCTTCGAGAACCATTTGCTTCCCGCTCAGACGTCTTGAGTGCTACAGGAAG
CTGGCACCACTACTTCAGAGAACAAGGCCTTTTCCTCTCCTCGCTCCAGT
exemplary donor template for insertion at GAPDH locus
SEQ ID NO: 206
GTCGACGAAGACTGTGGATGGCCCCTCCGGGAAACTGTGGCGTGATGGCCGCGGGGCTCTCCAG
AACATCATCCCTGCCTCTACTGGCGCTGCCAAGGCTGTGGGCAAGGTCATCCCTGAGCTGAACG
GGAAGCTCACTGGCATGGCCTTCCGTGTCCCCACTGCCAACGTGTCAGTGGTGGACCTGACCTG
CCGTCTAGAAAAACCTGCCAAATATGATGACATCAAGAAGGTGGTGAAGCAGGCGTCGGAGGGC
CCCCTCAAGGGCATCCTGGGCTACACTGAGCACCAGGTGGTCTCCTCTGACTTCAACAGCGACA
CCCACTCCTCCACCTTTGACGCTGGGGCTGGCATTGCCCTCAACGACCACTTTGTCAAGCTCAT
TTCCTGGTATGTGGCTGGGGCCAGAGACTGGCTCTTAAAAAGTGCAGGGTCTGGCGCCCTCTGG
TGGCTGGCTCAGAAAAAGGGCCCTGACAACTCTTTACATCTTCTAGGTATGACAACGAGTTCGG
ATATAGCAATAGAGTGGTCGATCTGATGGCTCATATGGCTAGCAAAGAGGGAAGCGGAGCTACT
AACTTCAGCCTGCTGAAGCAGGCTGGAGACGTGGAGGAGAACCCTGGACCTATGCTTCTCCTGG
TGACAAGCCTTCTGCTCTGTGAGTTACCACACCCAGCATTCCTCCTGATCCCAGACATCCAGAT
GACACAGACTACATCCTCCCTGTCTGCCTCTCTGGGAGACAGAGTCACCATCAGTTGCAGGGCA
AGTGAGGACATTAGTAAATATTTAAATTGGTATGAGCAGAAACCAGATGGAACTGTTAAACTCC
TGATCTACCATACATCAAGATTACACTCAGGAGTCCCATCAAGGTTCAGTGGCAGTGGGTCTGG
AACAGATTATTCTCTCACCATTAGCAACCTGGAGCAAGAAGATATTGCCACTTAGTTTTGCCAA
CAGGGTAATACGCTTCCGTACACGTTCGGAGGGGGGACTAAGTTGGAAATAACAGGCTCCACCT
CTGGATCCGGCAAGCCCGGATCTGGCGAGGGATCCACCAAGGGCGAGGTGAAACTGCAGGAGTC
AGGACCTGGCCTGGTGGCGCCCTCACAGAGCCTGTCCGTCACATGCACTGTCTCAGGGGTCTCA
TTACCCGACTATGGTGTAAGCTGGATTCGCCAGCCTCCACGAAAGGGTCTGGAGTGGCTGGGAG
TAATATGGGGTAGTGAAACCACATACTATAATTCAGCTCTCAAATCCAGACTGACCATCATCAA
GGACAACTCCAAGAGCCAAGTTTTCTTAAAAATGAACAGTCTGCAAACTGATGACACAGCCATT
TACTACTGTGCCAAACATTATTACTACGGTGGTAGCTATGCTATGGACTACTGGGGTCAAGGAA
CCTCAGTCACCGTCTCCTCAGCGGCCGCAATTGAAGTTATGTATCCTCCTCCTTACCTAGACAA
TGAGAAGAGCAATGGAACCATTATCCATGTGAAAGGGAAACACCTTTGTCCAAGTCCCCTATTT
CCCGGACCTTCTAAGCCCTTTTGGGTGCTGGTGGTGGTTGGGGGAGTCCTGGCTTGCTATAGCT
TGCTAGTAACAGTGGCCTTTATTATTTTCTGGGTGAGGAGTAAGAGGAGCAGGCTCCTGCACAG
TGACTACATGAACATGACTCCCCGCCGCCCCGGGCCCACCCGCAAGCATTACCAGCCCTATGCC
CCACCACGCGACTTCGCAGCCTATCGCTCCAGAGTGAAGTTCAGCAGGAGCGCAGACGCCCCCG
CGTACCAGCAGGGCCAGAACCAGCTCTATAACGAGCTCAATCTAGGACGAAGAGAGGAGTACGA
TGTTTTGGACAAGAGACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTACAGTGAGATTGGGA
TGAAAGGCGAGCGCCGGAGGGGCAAGGGGCACGATGGCCTTTACCAGGGTCTCAGTACAGCCAC
CAAGGACACCTACGACGCCCTTCACATGCAGGCCCTGCCCCCTCGCTAAAGCGGCCGCGTCGAG
TCTAGAGGGCCCGTTTAAACCCGCTGATCAGCCTCGACTGTGCCTTCTAGTTGCCAGCCATCTG
TTGTTTGCCCCTCCCCCGTGCCTTCCTTGACCCTGGAAGGTGCCACTCCCACTGTCCTTTCCTA
ATAAAATGAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGATGCGGTGGGCT
CTATGGATTTGGCTACAGCAACAGGGTGGTGGACCTCATGGCCCACATGGCCTCCAAGGAGTAA
GACCCCTGGACCACCAGCCCCAGCAAGAGCACAAGAGGAAGAGAGAGACCCTCACTGCTGGGGA
GTCCCTGCCACACTCAGTCCCCCACCACACTGAATCTCCCCTCCTCACAGTTGCCATGTAGACC
CCTTGAAGAGGGGAGGGGCCTAGGGAGCCGCACCTTGTCATGTACCATCAATAAAGTACCCTGT
GCTCAACCAGTTACTTGTCCTGTCTTATTCTAGGGTCTGGGGCAGAGGGGAGGGAAGCTGGGCT
TGTGTCAAGGTGAGACATTCTTGCTGGGGAGGGACCTGGTATGTTCTCCTCAGACTGAGGGTAG
GGCCTCCAAACAGCCTTGCTTGCTTCGAGAACCATTTGCTTCCCGCTCAGACGTCTTGAGTGCT
ACAGGAAGCTGGCACCACTACTTCAGAGAACAAGGCCTTTTCCTCTCCTCGCTCCAGT
exemplary donor template for insertion at GAPDH locus
SEQ ID NO: 207
GTCGACGAAGACTGTGGATGGCCCCTCCGGGAAACTGTGGCGTGATGGCCGCGGGGCTCTCCAG
AACATCATCCCTGCCTCTACTGGCGCTGCCAAGGCTGTGGGCAAGGTCATCCCTGAGCTGAACG
GGAAGCTCACTGGCATGGCCTTCCGTGTCCCCACTGCCAACGTGTCAGTGGTGGACCTGACCTG
CCGTCTAGAAAAACCTGCCAAATATGATGACATCAAGAAGGTGGTGAAGCAGGCGTCGGAGGGC
CCCCTCAAGGGCATCCTGGGCTACACTGAGCACCAGGTGGTCTCCTCTGACTTCAACAGCGACA
CCCACTCCTCCACCTTTGACGCTGGGGCTGGCATTGCCCTCAACGACCACTTTGTCAAGCTCAT
TTCCTGGTATGTGGCTGGGGCCAGAGACTGGCTCTTAAAAAGTGCAGGGTCTGGCGCCCTCTGG
TGGCTGGCTCAGAAAAAGGGCCCTGACAACTCTTTACATCTTCTAGGTATGACAACGAGTTCGG
ATATAGCAATAGAGTGGTCGATCTGATGGCTCATATGGCTAGCAAAGAGGGAAGCGGAGCTACT
AACTTCAGCCTGCTGAAGCAGGCTGGAGACGTGGAGGAGAACCCTGGACCTATGGCACTCCCCG
TCACCGCCCTTCTCTTGCCCCTCGCCCTGCTGCTGCATGCTGCCAGGCCCATGGACGAAGTGCA
GCTCGTGGAGTCCGGTGGAGGACTCGTCCAACCGGGCGGATCCCTTCGCTTGTCCTGCGCCGCA
TCAGGCTTCAGCTTCACCAACTATGGCGTCCACTGGGTCAGACAGGCCCCCGGAAAGGGACTGG
AATGGGTGTCCGTGATCTGGAGCGGCGGGAACACCGACTACAACACCTCCGTGAAGGGCCGGTT
CACTATTAGCCGCGACAACTCCAAGAACACTCTGTACCTCCAAATGAACTCCCTGAGGGCCGAA
GATACTGCTGTGTACTATTGCGCGAGAGCCCTGACCTACTACGACTACGAGTTCGCGTACTGGG
GCCAGGGGACTCTCGTGACCGTGTCCAGCGGTGGTGGAGGTTCCGGAGGCGGAGGTTCTGGTGG
CGGGGGATCAGAAATCGTGCTGACTCAGTCCCCTGCGACCTTGTCCCTGAGCCCTGGAGAACGG
GCCACCCTGAGCTGTAGAGCCAGCCAGAGCATCGGGACAAATATTCACTGGTACCAGCAGAAAC
CCGGACAAGCACCACGGCTGCTGATCTACTACGCCTCCGAGTCGATTTCCGGAATCCCGGCTCG
CTTTTCGGGGTCTGGATCGGGAACGGACTTCACTCTGACCATCTCGTCGCTGGAACCCGAGGAT
TTCGCCGTGTACTACTGCCAACAGAACAACAATTGGCCGACCACGTTCGGCCAGGGCACCAAGC
TCGAGATTAAGGGATCACTGGAAGCGGCCGCAACCACAACACCTGCTCCAAGGCCCCCCACACC
CGCTCCAACTATAGCCAGCCAACCATTGAGCCTCAGACCTGAAGCTTGCAGGCCCGCAGCAGGA
GGCGCCGTCCATACGCGAGGCCTGGACTTCGCGTGTGATATTTATATTTGGGCCCCTTTGGCCG
GAACATGTGGGGTGTTGCTTCTCTCCCTTGTGATCACTCTGTATTGTAAGCGCGGGAGAAAGAA
GCTCCTGTACATCTTCAAGCAGCCTTTTATGCGACCTGTGCAAACCACTCAGGAAGAAGATGGG
TGTTCATGCCGCTTCCCCGAGGAGGAAGAAGGAGGGTGTGAACTGAGGGTGAAATTTTCTAGAA
GCGCCGATGCTCCCGCATATCAGCAGGGTCAGAATCAGCTCTACAATGAATTGAATCTCGGCAG
GCGAGAAGAGTACGATGTTCTGGACAAGAGACGGGGCAGGGATCCCGAGATGGGGGGAAAGCCC
CGGAGAAAAAATCCTCAGGAGGGGTTGTACAATGAGCTGCAGAAGGACAAGATGGCTGAAGCCT
ATAGCGAGATCGGAATGAAAGGCGAAAGACGCAGAGGCAAGGGGCATGACGGTCTGTACCAGGG
TCTCTCTACAGCCACCAAGGACACTTATGATGCGTTGCATATGCAAGCCTTGCCACCCCGCTAA
AGCGGCCGCGTCGAGTCTAGAGGGCCCGTTTAAACCCGCTGATCAGCCTCGACTGTGCCTTCTA
GTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATAGCAGGCATGCTG
GGGATGCGGTGGGCTCTATGGATTTGGCTACAGCAACAGGGTGGTGGACCTCATGGCCCACATG
GCCTCCAAGGAGTAAGACCCCTGGACCACCAGCCCCAGCAAGAGCACAAGAGGAAGAGAGAGAC
CCTCACTGCTGGGGAGTCCCTGCCACACTCAGTCCCCCACCACACTGAATCTCCCCTCCTCACA
GTTGCCATGTAGACCCCTTGAAGAGGGGAGGGGCCTAGGGAGCCGCACCTTGTCATGTACCATC
AATAAAGTACCCTGTGCTCAACCAGTTACTTGTCCTGTCTTATTCTAGGGTCTGGGGCAGAGGG
GAGGGAAGCTGGGCTTGTGTCAAGGTGAGACATTCTTGCTGGGGAGGGACCTGGTATGTTCTCC
TCAGACTGAGGGTAGGGCCTCCAAACAGCCTTGCTTGCTTCGAGAACCATTTGCTTCCCGCTCA
GACGTCTTGAGTGCTACAGGAAGCTGGCACCACTACTTCAGAGAACAAGGCCTTTTCCTCTCCT
CGCTCCAGT
exemplary donor template for insertion at GAPDH locus
SEQ ID NO: 208
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGATTGGACCTGGATCC
TGTTTCTGGTGGCCGCTGCCACAAGAGTGCACAGCAATTGGGTCAACGTGATCAGCGACCTGAA
GAAGATCGAGGAGCTGATCCAGAGCATGCACATCGAGGCCACACTGTAGACCGAGTCCGATGTG
CACCCTAGCTGCAAAGTGACCGCCATGAAGTGCTTTCTGCTGGAACTGCAAGTGATCAGCCTGG
AAAGCGGCGACGCCAGCATCCACGATACCGTGGAAAACCTGATCATCCTGGCCAACAACAGCCT
GAGCAGCAACGGCAATGTGACCGAGAGCGGCTGCAAAGAGTGCGAGGAACTGGAAGAGAAGAAC
ATCAAAGAGTTCCTCCAGAGCTTCGTCCACATCGTGCAGATGTTCATCAACACCAGCGGAAGCG
GAGCCACAAACTTCTCTCTGCTGAAGCAGGCAGGAGATGTTGAAGAAAACCCTGGACCTATCAC
CTGTCCTCCACCTATGAGCGTGGAACACGCCGACATCTGGGTCAAGAGCTACAGCCTGTACAGC
AGAGAGCGGTACATCTGCAACAGCGGCTTCAAGAGAAAGGCCGGCACAAGCAGCCTGACCGAGT
GTGTGCTGAACAAGGCCACAAACGTGGCCCACTGGACCACACCTAGCCTGAAGTGCATCAGAGA
TCCCGCTCTGGTTCATCAGAGGCCTGCCCCTCCATCTACAGTGACAACAGCTGGCGTGACCCCT
CAGCCTGAGTCTCTGTCTCCATCTGGAAAAGAGCCTGCCGCCAGCTCTCCCAGCTCTAACAATA
CTGCTGCCACCACAGCCGCTATCGTGCCTGGATCTCAGCTGATGCCTAGCAAGAGCCCTAGCAC
CGGCACAACAGAGATCAGCTCTCACGAGAGCAGCCACGGAACACCTTCTCAGACCACCGCCAAG
AATTGGGAGCTGACAGCCTCTGCCTCTCATCAGCCACCTGGCGTGTACCCACAGGGCCACTCTG
ATACAACAGTGGCCATCAGCACCAGCACCGTTCTGCTGTGTGGCCTGTCTGCTGTTAGCCTGCT
GGCCTGCTACCTGAAGTCTAGACAGACACCTCCTCTGGCCAGCGTGGAAATGGAAGCCATGGAA
GCTCTGCCTGTCACATGGGGCACCAGCAGCAGAGATGAGGACCTCGAGAATTGCAGCCACCACC
TGGGAAGCGGAGCCACAAACTTCTCTCTGCTGAAGCAGGCAGGAGATGTTGAAGAAAACCCTGG
ACCTATGGTGAGCAAGGGCGAGGAGCTGTTCACCGGGGTGGTGCCCATCCTGGTCGAGCTGGAC
GGCGACGTAAACGGCCACAAGTTCAGCGTGTCCGGCGAGGGCGAGGGCGATGCCACCTACGGCA
AGCTGACCCTGAAGTTCATCTGCACCACCGGCAAGCTGCCCGTGCCCTGGCCCACCCTCGTGAC
CACCCTGACCTACGGCGTGCAGTGCTTCAGCCGCTACCCCGACCACATGAAGCAGCACGACTTC
TTCAAGTCCGCCATGCCCGAAGGCTACGTCCAGGAGCGCACCATCTTCTTCAAGGACGACGGCA
ACTACAAGACCCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAACTACAACAGC
CACAACGTCTATATCATGGCCGACAAGCAGAAGAACGGCATCAAGGTGAACTTCAAGATCCGCC
ACAACATCGAGGACGGCAGCGTGCAGCTCGCCGACCACTACCAGCAGAACACCCCCATCGGCGA
CGGCCCCGTGCTGCTGCCCGACAACCACTACCTGAGCACCCAGTCCGCCCTGAGCAAAGACCCC
AACGAGAAGCGCGATCACATGGTCCTGCTGGAGTTCGTGACCGCCGCCGGGATCACTCTCGGCA
TGGACGAGCTGTACAAGTAAGCGGCCGCGTCGAGTCTAGAGGGCCCGTTTAAACCCGCTGATCA
GCCTCGACTGTGCCTTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATTGCATCGCATTGTCT
GAGTAGGTGTCATTCTATTCTGGGGGGTGGGGTGGGGCAGGACAGCAAGGGGGAGGATTGGGAA
GACAATAGCAGGCATGCTGGGGATGCGGTGGGCTCTATGGATTTGGCTACAGCAACAGGGTGGT
GGACCTCATGGCCCACATGGCCTCCAAGGAGTAAGACCCCTGGACCACCAGCCCCAGCAAGAGC
ACAAGAGGAAGAGAGAGACCCTCACTGCTGGGGAGTCCCTGCCACACTCAGTCCCCCACCACAC
TGAATCTCCCCTCCTCACAGTTGCCATGTAGACCCCTTGAAGAGGGGAGGGGCCTAGGGAGCCG
CACCTTGTCATGTACCATCAATAAAGTACCCTGTGCTCAACCAGTTACTTGTCCTGTCTTATTC
TAGGGTCTGGGGCAGAGGGGAGGGAAGCTGGGCTTGTGTCAAGGTGAGACATTCTTGCTGGGGA
GGGACCTGGTATGTTCTCCTCAGACTGAGGGTAGGGCCTCCAAACAGCCTTGCTTGCTTCGAGA
ACCATTTGCTTCCCGCTCAGACGTCTTGAGTGCTACAGGAAGCTGGCACCACTACTTCAGAGAA
CAAGGCCTTTTCCTCTCCTCGCTCCAGT
exemplary donor template for insertion at GAPDH locus
SEQ ID NO: 209
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTGGCAGCTGTTGCTGC
CGACAGCCCTCCTGTTGCTGGTCTCCGCTGGCATGAGAACCGAGGATCTGCCTAAGGCCGTGGT
GTTCCTGGAACCTCAGTGGTACAGAGTGCTGGAAAAGGACAGCGTGACCCTGAAGTGCCAGGGC
GCCTATTCTCCCGAGGACAATAGCACCCAGTGGTTCCACAACGAGAGCCTGATCAGCAGCCAGG
CCAGCAGCTACTTTATCGATGCCGCCACCGTGGACGACAGCGGCGAGTACAGATGCCAGACCAA
TCTGAGCACCCTGAGCGACCCTGTGCAGCTGGAAGTGCACATTGGATGGTTGCTGCTGCAAGCC
CCTAGATGGGTGTTCAAAGAAGAGGACCCCATCCACCTGAGATGCCACTCTTGGAAGAACACAG
CCCTGCACAAAGTGACCTACCTGCAGAACGGCAAGGGCAGAAAGTACTTCCACCACAACAGCGA
CTTCTACATCCCCAAGGCCACACTGAAGGACTCCGGCTCCTACTTCTGCAGAGGCCTGGTCGGC
AGCAAGAACGTGTCCAGCGAGACAGTGAACATCACCATCACACAGGGCCTCGCCGTGTCTACCA
TCAGCAGCTTTTTCCCACCTGGCTATCAGGTGTCCTTCTGCCTGGTCATGGTGCTGCTGTTCGC
CGTGGATACCGGCCTGTACTTCAGCGTCAAGACCAACATCCGGTCCAGCACCAGAGACTGGAAG
GACCACAAGTTCAAGTGGCGGAAGGACCCTCAGGACAAGGGAAGCGGAGCCACAAACTTCTCTC
TGCTGAAGCAGGCAGGAGATGTTGAAGAAAACCCTGGACCTATGGATTGGACCTGGATCCTGTT
TCTGGTGGCCGCTGCCACAAGAGTGCACAGCAATTGGGTCAACGTGATCAGCGACCTGAAGAAG
ATCGAGGACCTGATCCAGAGCATGCACATCGACGCCACACTGTACACCGAGTCCGATGTGCACC
CTAGCTGCAAAGTGACCGCCATGAAGTGCTTTCTGCTGGAACTGCAAGTGATCAGCCTGGAAAG
CGGCGACGCCAGCATCCACGATACCGTGGAAAACCTGATCATCCTGGCCAACAACAGCCTGAGC
AGCAACGGCAATGTGACCGAGAGCGGCTGCAAAGAGTGCGAGGAACTGGAAGAGAAGAACATCA
AAGAGTTCCTCCAGAGCTTCGTCCACATCGTGCAGATGTTCATCAACACCAGCGGAAGCGGAGC
CACAAACTTCTCTCTGCTGAAGCAGGCAGGAGATGTTGAAGAAAACCCTGGACCTATCACCTGT
CCTCCACCTATGAGCGTGGAACACGCCGACATCTGGGTCAAGAGCTACAGCCTGTACAGCAGAG
AGCGGTACATCTGCAACAGCGGCTTCAAGAGAAAGGCCGGCACAAGCAGCCTGACCGAGTGTGT
GCTGAACAAGGCCACAAACGTGGCCCACTGGACCACACCTAGCCTGAAGTGCATCAGAGATCCC
GCTCTGGTTCATCAGAGGCCTGCCCCTCCATCTACAGTGACAACAGCTGGCGTGACCCCTCAGC
CTGAGTCTCTGTCTCCATCTGGAAAAGAGCCTGCCGCCAGCTCTCCCAGCTCTAACAATACTGC
TGCCACCACAGCCGCTATCGTGCCTGGATCTCAGCTGATGCCTAGCAAGAGCCCTAGCACCGGC
ACAACAGAGATCAGCTCTCACGAGAGCAGCCACGGAACACCTTCTCAGACCACCGCCAAGAATT
GGGAGCTGACAGCCTCTGCCTCTCATCAGCCACCTGGCGTGTACCCACAGGGCCACTCTGATAC
AACAGTGGCCATCAGCACCAGCACCGTTCTGCTGTGTGGCCTGTCTGCTGTTAGCCTGCTGGCC
TGCTACCTGAAGTCTAGACAGACACCTCCTCTGGCCAGCGTGGAAATGGAAGCCATGGAAGCTC
TGCCTGTCACATGGGGCACCAGCAGCAGAGATGAGGACCTCGAGAATTGCAGCCACCACCTGTA
AGCGGCCGCGTCGAGTCTAGAGGGCCCGTTTAAACCCGCTGATCAGCCTCGACTGTGCCTTCTA
GTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATAGCAGGCATGCTG
GGGATGCGGTGGGCTCTATGGATTTGGCTACAGCAACAGGGTGGTGGACCTCATGGCCCACATG
GCCTCCAAGGAGTAAGACCCCTGGACCACCAGCCCCAGCAAGAGCACAAGAGGAAGAGAGAGAC
CCTCACTGCTGGGGAGTCCCTGCCACACTCAGTCCCCCACCACACTGAATCTCCCCTCCTCACA
GTTGCCATGTAGACCCCTTGAAGAGGGGAGGGGCCTAGGGAGCCGCACCTTGTCATGTACCATC
AATAAAGTACCCTGTGCTCAACCAGTTACTTGTCCTGTCTTATTCTAGGGTCTGGGGCAGAGGG
GAGGGAAGCTGGGCTTGTGTCAAGGTGAGACATTCTTGCTGGGGAGGGACCTGGTATGTTCTCC
TCAGACTGAGGGTAGGGCCTCCAAACAGCCTTGCTTGCTTCGAGAACCATTTGCTTCCCGCTCA
GACGTCTTGAGTGCTACAGGAAGCTGGCACCACTACTTCAGAGAACAAGGCCTTTTCCTCTCCT
CGCTCCAGT
exemplary donor template for insertion at GAPDH locus
SEQ ID NO: 210
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGATTGGACCTGGATCC
TGTTTCTGGTGGCCGCTGCCACAAGAGTGCACAGCAATTGGGTCAACGTGATCAGCGACCTGAA
GAAGATCGAGGAGCTGATCCAGAGCATGGAGATCGAGGCCACACTGTAGACCGAGTCCGATGTG
CACCCTAGCTGCAAAGTGACCGCCATGAAGTGCTTTCTGCTGGAACTGCAAGTGATCAGCCTGG
AAAGCGGCGACGCCAGCATCCACGATACCGTGGAAAACCTGATCATCCTGGCCAACAACAGCCT
GAGCAGCAACGGCAATGTGACCGAGAGCGGCTGCAAAGAGTGCGAGGAACTGGAAGAGAAGAAC
ATCAAAGAGTTCCTCCAGAGCTTCGTCCACATCGTGCAGATGTTCATCAACACCAGCTCTGGCG
GAGGAAGCGGAGGCGGAGGATCTGGTGGTGGTGGATCTGGCGGCGGTGGTAGTGGCGGAGGTTC
TCTGCAAATCACCTGTCCTCCACCTATGAGCGTGGAACACGCCGACATCTGGGTCAAGAGCTAC
AGCCTGTACAGCAGAGAGCGGTACATCTGCAACAGCGGCTTCAAGAGAAAGGCCGGCACAAGCA
GCCTGACCGAGTGTGTGCTGAACAAGGCCACAAACGTGGCCCACTGGACCACACCTAGCCTGAA
GTGCATCAGAGATCCCGCTCTGGTTCATCAGAGGCCTGCCCCTCCATCTACAGTGACAACAGCT
GGCGTGACCCCTCAGCCTGAGTCTCTGTCTCCATCTGGAAAAGAGCCTGCCGCCAGCTCTCCCA
GCTCTAACAATACTGCTGCCACCACAGCCGCTATCGTGCCTGGATCTCAGCTGATGCCTAGCAA
GAGCCCTAGCACCGGCACAACAGAGATCAGCTCTCACGAGAGCAGCCACGGAACACCTTCTCAG
ACCACCGCCAAGAATTGGGAGCTGACAGCCTCTGCCTCTCATCAGCCACCTGGCGTGTACCCAC
AGGGCCACTCTGATACAACAGTGGCCATCAGCACCAGCACCGTTCTGCTGTGTGGCCTGTCTGC
TGTTAGCCTGCTGGCCTGCTACCTGAAGTCTAGACAGACACCTCCTCTGGCCAGCGTGGAAATG
GAAGCCATGGAAGCTCTGCCTGTCACATGGGGCACCAGCAGCAGAGATGAGGACCTCGAGAATT
GCAGCCACCACCTGGGAAGCGGAGCCACAAACTTCTCTCTGCTGAAGCAGGCAGGAGATGTTGA
AGAAAACCCTGGACCTATGTGGCAGCTGTTGCTGCCGACAGCCCTCCTGTTGCTGGTCTCCGCT
GGCATGAGAACCGAGGATCTGCCTAAGGCCGTGGTGTTCCTGGAACCTCAGTGGTACAGAGTGC
TGGAAAAGGACAGCGTGACCCTGAAGTGCCAGGGCGCCTATTCTCCCGAGGACAATAGCACCCA
GTGGTTCCACAACGAGAGCCTGATCAGCAGCCAGGCCAGCAGCTACTTTATCGATGCCGCCACC
GTGGACGACAGCGGCGAGTACAGATGCCAGACCAATCTGAGCACCCTGAGCGACCCTGTGCAGC
TGGAAGTGCACATTGGATGGTTGCTGCTGCAAGCCCCTAGATGGGTGTTCAAAGAAGAGGACCC
CATCCACCTGAGATGCCACTCTTGGAAGAACACAGCCCTGCACAAAGTGACCTACCTGCAGAAC
GGCAAGGGCAGAAAGTACTTCCACCACAACAGCGACTTCTACATCCCCAAGGCCACACTGAAGG
ACTCCGGCTCCTACTTCTGCAGAGGCCTGGTCGGCAGCAAGAACGTGTCCAGCGAGACAGTGAA
CATCACCATCACACAGGGCCTCGCCGTGTCTACCATCAGCAGCTTTTTCCCACCTGGCTATCAG
GTGTCCTTCTGCCTGGTCATGGTGCTGCTGTTCGCCGTGGATACCGGCCTGTACTTCAGCGTCA
AGACCAACATCCGGTCCAGCACCAGAGACTGGAAGGACCACAAGTTCAAGTGGCGGAAGGACCC
TCAGGACAAGTAAGCGGCCGCGTCGAGTCTAGAGGGCCCGTTTAAACCCGCTGATCAGCCTCGA
CTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGACAATA
GCAGGCATGCTGGGGATGCGGTGGGCTCTATGGATTTGGCTACAGCAACAGGGTGGTGGACCTC
ATGGCCCACATGGCCTCCAAGGAGTAAGACCCCTGGACCACCAGCCCCAGCAAGAGCACAAGAG
GAAGAGAGAGACCCTCACTGCTGGGGAGTCCCTGCCACACTCAGTCCCCCACCACACTGAATCT
CCCCTCCTCACAGTTGCCATGTAGACCCCTTGAAGAGGGGAGGGGCCTAGGGAGCCGCACCTTG
TCATGTACCATCAATAAAGTACCCTGTGCTCAACCAGTTACTTGTCCTGTCTTATTCTAGGGTC
TGGGGCAGAGGGGAGGGAAGCTGGGCTTGTGTCAAGGTGAGACATTCTTGCTGGGGAGGGACCT
GGTATGTTCTCCTCAGACTGAGGGTAGGGCCTCCAAACAGCCTTGCTTGCTTCGAGAACCATTT
GCTTCCCGCTCAGACGTCTTGAGTGCTACAGGAAGCTGGCACCACTACTTCAGAGAACAAGGCC
TTTTCCTCTCCTCGCTCCAGT
exemplary donor template for insertion at GAPDH locus
SEQ ID NO: 211
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGATTGGACCTGGATCC
TGTTTCTGGTGGCCGCTGCCACAAGAGTGCACAGCAATTGGGTCAACGTGATCAGCGACCTGAA
GAAGATCGAGGAGCTGATCCAGAGCATGGAGATCGAGGCCACACTGTAGACCGAGTCCGATGTG
CACCCTAGCTGCAAAGTGACCGCCATGAAGTGCTTTCTGCTGGAACTGCAAGTGATCAGCCTGG
AAAGCGGCGACGCCAGCATCCACGATACCGTGGAAAACCTGATCATCCTGGCCAACAACAGCCT
GAGCAGCAACGGCAATGTGACCGAGAGCGGCTGCAAAGAGTGCGAGGAACTGGAAGAGAAGAAC
ATCAAAGAGTTCCTCCAGAGCTTCGTCCACATCGTGCAGATGTTCATCAACACCAGCGGAAGCG
GAGCCACAAACTTCTCTCTGCTGAAGCAGGCAGGAGATGTTGAAGAAAACCCTGGACCTATCAC
CTGTCCTCCACCTATGAGCGTGGAACACGCCGACATCTGGGTCAAGAGCTACAGCCTGTACAGC
AGAGAGCGGTACATCTGCAACAGCGGCTTCAAGAGAAAGGCCGGCACAAGCAGCCTGACCGAGT
GTGTGCTGAACAAGGCCACAAACGTGGCCCACTGGACCACACCTAGCCTGAAGTGCATCAGAGA
TCCCGCTCTGGTTCATCAGAGGCCTGCCCCTCCATCTACAGTGACAACAGCTGGCGTGACCCCT
CAGCCTGAGTCTCTGTCTCCATCTGGAAAAGAGCCTGCCGCCAGCTCTCCCAGCTCTAACAATA
CTGCTGCCACCACAGCCGCTATCGTGCCTGGATCTCAGCTGATGCCTAGCAAGAGCCCTAGCAC
CGGCACAACAGAGATCAGCTCTCACGAGAGCAGCCACGGAACACCTTCTCAGACCACCGCCAAG
AATTGGGAGCTGACAGCCTCTGCCTCTCATCAGCCACCTGGCGTGTACCCACAGGGCCACTCTG
ATACAACAGTGGCCATCAGCACCAGCACCGTTCTGCTGTGTGGCCTGTCTGCTGTTAGCCTGCT
GGCCTGCTACCTGAAGTCTAGACAGACACCTCCTCTGGCCAGCGTGGAAATGGAAGCCATGGAA
GCTCTGCCTGTCACATGGGGCACCAGCAGCAGAGATGAGGACCTCGAGAATTGCAGCCACCACC
TGGGAAGCGGAGCCACAAACTTCTCTCTGCTGAAGCAGGCAGGAGATGTTGAAGAAAACCCTGG
ACCTATGTGGCAGCTGTTGCTGCCGACAGCCCTCCTGTTGCTGGTCTCCGCTGGCATGAGAACC
GAGGATCTGCCTAAGGCCGTGGTGTTCCTGGAACCTCAGTGGTACAGAGTGCTGGAAAAGGACA
GCGTGACCCTGAAGTGCCAGGGCGCCTATTCTCCCGAGGACAATAGCACCCAGTGGTTCCACAA
CGAGAGCCTGATCAGCAGCCAGGCCAGCAGCTACTTTATCGATGCCGCCACCGTGGACGACAGC
GGCGAGTACAGATGCCAGACCAATCTGAGCACCCTGAGCGACCCTGTGCAGCTGGAAGTGCACA
TTGGATGGTTGCTGCTGCAAGCCCCTAGATGGGTGTTCAAAGAAGAGGACCCCATCCACCTGAG
ATGCCACTCTTGGAAGAACACAGCCCTGCACAAAGTGACCTACCTGCAGAACGGCAAGGGCAGA
AAGTACTTCCACCACAACAGCGACTTCTACATCCCCAAGGCCACACTGAAGGACTCCGGCTCCT
ACTTCTGCAGAGGCCTGGTCGGCAGCAAGAACGTGTCCAGCGAGACAGTGAACATCACCATCAC
ACAGGGCCTCGCCGTGTCTACCATCAGCAGCTTTTTCCCACCTGGCTATCAGGTGTCCTTCTGC
CTGGTCATGGTGCTGCTGTTCGCCGTGGATACCGGCCTGTACTTCAGCGTCAAGACCAACATCC
GGTCCAGCACCAGAGACTGGAAGGACCACAAGTTCAAGTGGCGGAAGGACCCTCAGGACAAGTA
AGCGGCCGCGTCGAGTCTAGAGGGCCCGTTTAAACCCGCTGATCAGCCTCGACTGTGCCTTCTA
GTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATAGCAGGCATGCTG
GGGATGCGGTGGGCTCTATGGATTTGGCTACAGCAACAGGGTGGTGGACCTCATGGCCCACATG
GCCTCCAAGGAGTAAGACCCCTGGACCACCAGCCCCAGCAAGAGCACAAGAGGAAGAGAGAGAC
CCTCACTGCTGGGGAGTCCCTGCCACACTCAGTCCCCCACCACACTGAATCTCCCCTCCTCACA
GTTGCCATGTAGACCCCTTGAAGAGGGGAGGGGCCTAGGGAGCCGCACCTTGTCATGTACCATC
AATAAAGTACCCTGTGCTCAACCAGTTACTTGTCCTGTCTTATTCTAGGGTCTGGGGCAGAGGG
GAGGGAAGCTGGGCTTGTGTCAAGGTGAGACATTCTTGCTGGGGAGGGACCTGGTATGTTCTCC
TCAGACTGAGGGTAGGGCCTCCAAACAGCCTTGCTTGCTTCGAGAACCATTTGCTTCCCGCTCA
GACGTCTTGAGTGCTACAGGAAGCTGGCACCACTACTTCAGAGAACAAGGCCTTTTCCTCTCCT
CGCTCCAGT
exemplary donor template for insertion at GAPDH locus
SEQ ID NO: 212
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTGGCAGCTGTTGCTGC
CGACAGCCCTCCTGTTGCTGGTCTCCGCTGGCATGAGAACCGAGGATCTGCCTAAGGCCGTGGT
GTTCCTGGAACCTCAGTGGTACAGAGTGCTGGAAAAGGACAGCGTGACCCTGAAGTGCCAGGGC
GCCTATTCTCCCGAGGACAATAGCACCCAGTGGTTCCACAACGAGAGCCTGATCAGCAGCCAGG
CCAGCAGCTACTTTATCGATGCCGCCACCGTGGACGACAGCGGCGAGTACAGATGCCAGACCAA
TCTGAGCACCCTGAGCGACCCTGTGCAGCTGGAAGTGCACATTGGATGGTTGCTGCTGCAAGCC
CCTAGATGGGTGTTCAAAGAAGAGGACCCCATCCACCTGAGATGCCACTCTTGGAAGAACACAG
CCCTGCACAAAGTGACCTACCTGCAGAACGGCAAGGGCAGAAAGTACTTCCACCACAACAGCGA
CTTCTACATCCCCAAGGCCACACTGAAGGACTCCGGCTCCTACTTCTGCAGAGGCCTGGTCGGC
AGCAAGAACGTGTCCAGCGAGACAGTGAACATCACCATCACACAGGGCCTCGCCGTGTCTACCA
TCAGCAGCTTTTTCCCACCTGGCTATCAGGTGTCCTTCTGCCTGGTCATGGTGCTGCTGTTCGC
CGTGGATACCGGCCTGTACTTCAGCGTCAAGACCAACATCCGGTCCAGCACCAGAGACTGGAAG
GACCACAAGTTCAAGTGGCGGAAGGACCCTCAGGACAAGGGAAGCGGAGCCACAAACTTCTCTC
TGCTGAAGCAGGCAGGAGATGTTGAAGAAAACCCTGGACCTATGGATTGGACCTGGATCCTGTT
TCTGGTGGCCGCTGCCACAAGAGTGCACAGCAATTGGGTCAACGTGATCAGCGACCTGAAGAAG
ATCGAGGACCTGATCCAGAGCATGCACATCGACGCCACACTGTACACCGAGTCCGATGTGCACC
CTAGCTGCAAAGTGACCGCCATGAAGTGCTTTCTGCTGGAACTGCAAGTGATCAGCCTGGAAAG
CGGCGACGCCAGCATCCACGATACCGTGGAAAACCTGATCATCCTGGCCAACAACAGCCTGAGC
AGCAACGGCAATGTGACCGAGAGCGGCTGCAAAGAGTGCGAGGAACTGGAAGAGAAGAACATCA
AAGAGTTCCTCCAGAGCTTCGTCCACATCGTGCAGATGTTCATCAACACCAGCTCTGGCGGAGG
AAGCGGAGGCGGAGGATCTGGTGGTGGTGGATCTGGCGGCGGTGGTAGTGGCGGAGGTTCTCTG
CAAATCACCTGTCCTCCACCTATGAGCGTGGAACACGCCGACATCTGGGTCAAGAGCTACAGCC
TGTACAGCAGAGAGCGGTACATCTGCAACAGCGGCTTCAAGAGAAAGGCCGGCACAAGCAGCCT
GACCGAGTGTGTGCTGAACAAGGCCACAAACGTGGCCCACTGGACCACACCTAGCCTGAAGTGC
ATCAGAGATCCCGCTCTGGTTCATCAGAGGCCTGCCCCTCCATCTACAGTGACAACAGCTGGCG
TGACCCCTCAGCCTGAGTCTCTGTCTCCATCTGGAAAAGAGCCTGCCGCCAGCTCTCCCAGCTC
TAACAATACTGCTGCCACCACAGCCGCTATCGTGCCTGGATCTCAGCTGATGCCTAGCAAGAGC
CCTAGCACCGGCACAACAGAGATCAGCTCTCACGAGAGCAGCCACGGAACACCTTCTCAGACCA
CCGCCAAGAATTGGGAGCTGACAGCCTCTGCCTCTCATCAGCCACCTGGCGTGTACCCACAGGG
CCACTCTGATACAACAGTGGCCATCAGCACCAGCACCGTTCTGCTGTGTGGCCTGTCTGCTGTT
AGCCTGCTGGCCTGCTACCTGAAGTCTAGACAGACACCTCCTCTGGCCAGCGTGGAAATGGAAG
CCATGGAAGCTCTGCCTGTCACATGGGGCACCAGCAGCAGAGATGAGGACCTCGAGAATTGCAG
CCACCACCTGTAAGCGGCCGCGTCGAGTCTAGAGGGCCCGTTTAAACCCGCTGATCAGCCTCGA
CTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGACAATA
GCAGGCATGCTGGGGATGCGGTGGGCTCTATGGATTTGGCTACAGCAACAGGGTGGTGGACCTC
ATGGCCCACATGGCCTCCAAGGAGTAAGACCCCTGGACCACCAGCCCCAGCAAGAGCACAAGAG
GAAGAGAGAGACCCTCACTGCTGGGGAGTCCCTGCCACACTCAGTCCCCCACCACACTGAATCT
CCCCTCCTCACAGTTGCCATGTAGACCCCTTGAAGAGGGGAGGGGCCTAGGGAGCCGCACCTTG
TCATGTACCATCAATAAAGTACCCTGTGCTCAACCAGTTACTTGTCCTGTCTTATTCTAGGGTC
TGGGGCAGAGGGGAGGGAAGCTGGGCTTGTGTCAAGGTGAGACATTCTTGCTGGGGAGGGACCT
GGTATGTTCTCCTCAGACTGAGGGTAGGGCCTCCAAACAGCCTTGCTTGCTTCGAGAACCATTT
GCTTCCCGCTCAGACGTCTTGAGTGCTACAGGAAGCTGGCACCACTACTTCAGAGAACAAGGCC
TTTTCCTCTCCTCGCTCCAGT
exemplary donor template for insertion at GAPDH locus
SEQ ID NO: 213
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTGGCAGCTGTTGCTGC
CGACAGCCCTCCTGTTGCTGGTCTCCGCTGGCATGAGAACCGAGGATCTGCCTAAGGCCGTGGT
GTTCCTGGAACCTCAGTGGTACAGAGTGCTGGAAAAGGACAGCGTGACCCTGAAGTGCCAGGGC
GCCTATTCTCCCGAGGACAATAGCACCCAGTGGTTCCACAACGAGAGCCTGATCAGCAGCCAGG
CCAGCAGCTACTTTATCGATGCCGCCACCGTGGACGACAGCGGCGAGTACAGATGCCAGACCAA
TCTGAGCACCCTGAGCGACCCTGTGCAGCTGGAAGTGCACATTGGATGGTTGCTGCTGCAAGCC
CCTAGATGGGTGTTCAAAGAAGAGGACCCCATCCACCTGAGATGCCACTCTTGGAAGAACACAG
CCCTGCACAAAGTGACCTACCTGCAGAACGGCAAGGGCAGAAAGTACTTCCACCACAACAGCGA
CTTCTACATCCCCAAGGCCACACTGAAGGACTCCGGCTCCTACTTCTGCAGAGGCCTGGTCGGC
AGCAAGAACGTGTCCAGCGAGACAGTGAACATCACCATCACACAGGGCCTCGCCGTGTCTACCA
TCAGCAGCTTTTTCCCACCTGGCTATCAGGTGTCCTTCTGCCTGGTCATGGTGCTGCTGTTCGC
CGTGGATACCGGCCTGTACTTCAGCGTCAAGACCAACATCCGGTCCAGCACCAGAGACTGGAAG
GACCACAAGTTCAAGTGGCGGAAGGACCCTCAGGACAAGGGAAGCGGAGCCACAAACTTCTCTC
TGCTGAAGCAGGCAGGAGATGTTGAAGAAAACCCTGGACCTATGGATTGGACCTGGATCCTGTT
TCTGGTGGCCGCTGCCACAAGAGTGCACAGCAATTGGGTCAACGTGATCAGCGACCTGAAGAAG
ATCGAGGACCTGATCCAGAGCATGCACATCGACGCCACACTGTACACCGAGTCCGATGTGCACC
CTAGCTGCAAAGTGACCGCCATGAAGTGCTTTCTGCTGGAACTGCAAGTGATCAGCCTGGAAAG
CGGCGACGCCAGCATCCACGATACCGTGGAAAACCTGATCATCCTGGCCAACAACAGCCTGAGC
AGCAACGGCAATGTGACCGAGAGCGGCTGCAAAGAGTGCGAGGAACTGGAAGAGAAGAACATCA
AAGAGTTCCTCCAGAGCTTCGTCCACATCGTGCAGATGTTCATCAACACCAGCTCTGGCGGAGG
AAGCGGAGGCGGAGGATCTGGTGGTGGTGGATCTGGCGGCGGTGGTAGTGGCGGAGGTTCTCTG
CAAATCACCTGTCCTCCACCTATGAGCGTGGAACACGCCGACATCTGGGTCAAGAGCTACAGCC
TGTACAGCAGAGAGCGGTACATCTGCAACAGCGGCTTCAAGAGAAAGGCCGGCACAAGCAGCCT
GACCGAGTGTGTGCTGAACAAGGCCACAAACGTGGCCCACTGGACCACACCTAGCCTGAAGTGC
ATCAGAGATCCCGCTCTGGTTCATCAGAGGCCTGCCCCTCCATCTACAGTGACAACAGCTGGCG
TGACCCCTCAGCCTGAGTCTCTGTCTCCATCTGGAAAAGAGCCTGCCGCCAGCTCTCCCAGCTC
TAACAATACTGCTGCCACCACAGCCGCTATCGTGCCTGGATCTCAGCTGATGCCTAGCAAGAGC
CCTAGCACCGGCACAACAGAGATCAGCTCTCACGAGAGCAGCCACGGAACACCTTCTCAGACCA
CCGCCAAGAATTGGGAGCTGACAGCCTCTGCCTCTCATCAGCCACCTGGCGTGTACCCACAGGG
CCACTCTGATACAACAGTGGCCATCAGCACCAGCACCGTTCTGCTGTGTGGCCTGTCTGCTGTT
AGCCTGCTGGCCTGCTACCTGAAGTCTAGACAGACACCTCCTCTGGCCAGCGTGGAAATGGAAG
CCATGGAAGCTCTGCCTGTCACATGGGGCACCAGCAGCAGAGATGAGGACCTCGAGAATTGCAG
CCACCACCTGTAAGCGGCCGCGTCGAGTCTAGAGGGCCCGTTTAAACCCGCTGATCAGCCTCGA
CTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGACAATA
GCAGGCATGCTGGGGATGCGGTGGGCTCTATGGATTTGGCTACAGCAACAGGGTGGTGGACCTC
ATGGCCCACATGGCCTCCAAGGAGTAAGACCCCTGGACCACCAGCCCCAGCAAGAGCACAAGAG
GAAGAGAGAGACCCTCACTGCTGGGGAGTCCCTGCCACACTCAGTCCCCCACCACACTGAATCT
CCCCTCCTCACAGTTGCCATGTAGACCCCTTGAAGAGGGGAGGGGCCTAGGGAGCCGCACCTTG
TCATGTACCATCAATAAAGTACCCTGTGCTCAACCAGTTACTTGTCCTGTCTTATTCTAGGGTC
TGGGGCAGAGGGGAGGGAAGCTGGGCTTGTGTCAAGGTGAGACATTCTTGCTGGGGAGGGACCT
GGTATGTTCTCCTCAGACTGAGGGTAGGGCCTCCAAACAGCCTTGCTTGCTTCGAGAACCATTT
GCTTCCCGCTCAGACGTCTTGAGTGCTACAGGAAGCTGGCACCACTACTTCAGAGAACAAGGCC
TTTTCCTCTCCTCGCTCCAGT
exemplary donor template for insertion at GAPDH locus
SEQ ID NO: 214
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTCAAATATTACAGATC
CACAGATGTGGGATTTTGATGATCTAAATTTCACTGGCATGCCACCTGCAGATGAAGATTACAG
CCCCTGTATGCTAGAAACTGAGACACTCAACAAGTATGTTGTGATCATCGCCTATGCCCTAGTG
TTCCTGCTGAGCCTGCTGGGAAACTCCCTGGTGATGCTGGTCATCTTATACAGCAGGGTCGGCC
GCTCCGTCACTGATGTCTACCTGCTGAACCTGGCCTTGGCCGACCTACTCTTTGCCCTGACCTT
GCCCATCTGGGCCGCCTCCAAGGTGAATGGCTGGATTTTTGGCACATTCCTGTGCAAGGTGGTC
TCACTCCTGAAGGAAGTCAACTTCTACAGTGGCATCCTGCTGTTGGCCTGCATCAGTGTGGACC
GTTACCTGGCCATTGTCCATGCCACACGCACACTGACCCAGAAGCGTCACTTGGTCAAGTTTGT
TTGTCTTGGCTGCTGGGGACTGTCTATGAATCTGTCCCTGCCCTTCTTCCTTTTCCGCCAGGCT
TACCATCCAAACAATTCCAGTCCAGTTTGCTATGAGGTCCTGGGAAATGACACAGCAAAATGGC
GGATGGTGTTGCGGATCCTGCCTCACACCTTTGGCTTCATCGTGCCGCTGTTTGTCATGCTGTT
CTGCTATGGATTCACCCTGCGTACACTGTTTAAGGCCCACATGGGGCAGAAGCACCGAGCCATG
AGGGTCATCTTTGCTGTCGTCCTCATCTTCCTGCTTTGCTGGCTGCCCTACAACCTGGTCCTGC
TGGCAGACACCCTCATGAGGACCCAGGTGATCCAGGAGAGCTGTGAGCGCCGCAACAACATCGG
CCGGGCCCTGGATGCCACTGAGATTCTGGGATTTCTCCATAGCTGCCTCAACCCCATCATCTAC
GCCTTCATCGGCCAAAATTTTCGCCATGGATTCCTCAAGATCCTGGCTATGCATGGCCTGGTCA
GCAAGGAGTTCTTGGCACGTCATCGTGTTACCTCCTACACTTCTTCGTCTGTCAATGTCTCTTC
CAACCTCTGAATTTGGCTACAGCAACAGGGTGGTGGACCTCATGGCCCACATGGCCTCCAAGGA
GTAAGACCCCTGGACCACCAGCCCCAGCAAGAGCACAAGAGGAAGAGAGAGACCCTCACTGCTG
GGGAGTCCCTGCCACACTCAGTCCCCCACCACACTGAATCTCCCCTCCTCACAGTTGCCATGTA
GACCCCTTGAAGAGGGGAGGGGCCTAGGGAGCCGCACCTTGTCATGTACCATCAATAAAGTACC
CTGTGCTCAACCAGTTACTTGTCCTGTCTTATTCTAGGGTCTGGGGCAGAGGGGAGGGAAGCTG
GGCTTGTGTCAAGGTGAGACATTCTTGCTGGGGAGGGACCTGGTATGTTCTCCTCAGACTGAGG
GTAGGGCCTCCAAACAGCCTTGCTTGCTTCGAGAACCATTTGCTTCCCGCTCAGACGTCTTGAG
TGCTACAGGAAGCTGGCACCACTACTTCAGAGAACAAGGCCTTTTCCTCTCCTCGCTCCAGT
exemplary donor template for insertion at GAPDH locus
SEQ ID NO: 215
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGAGTTGAGGAAGTACG
GCCCTGGAAGACTGGCGGGGACAGTTATAGGAGGAGCTGCTCAGAGTAAATCACAGACTAAATC
AGACTCAATCACAAAAGAGTTCCTGCCAGGCCTTTACACAGCCCCTTCCTCCCCGTTCCCGCCC
TCACAGGTGAGTGACCACCAAGTGCTAAATGACGCCGAGGTTGCCGCCCTCCTGGAGAACTTCA
GCTCTTCCTATGACTATGGAGAAAACGAGAGTGACTCGTGCTGTACCTCCCCGCCCTGCCCACA
GGACTTCAGCCTGAACTTCGACCGGGCCTTCCTGCCAGCCCTCTACAGCCTCCTCTTTCTGCTG
GGGCTGCTGGGCAACGGCGCGGTGGCAGCCGTGCTGCTGAGCCGGCGGACAGCCCTGAGCAGCA
CCGACACCTTCCTGCTCCACCTAGCTGTAGCAGACACGCTGCTGGTGCTGACACTGCCGCTCTG
GGCAGTGGACGCTGCCGTCCAGTGGGTCTTTGGCTCTGGCCTCTGCAAAGTGGCAGGTGCCCTC
TTCAACATCAACTTCTACGCAGGAGCCCTCCTGCTGGCCTGCATCAGCTTTGACCGCTACCTGA
ACATAGTTCATGCCACCCAGCTCTACCGCCGGGGGCCCCCGGCCCGCGTGACCCTCACCTGCCT
GGCTGTCTGGGGGCTCTGCCTGCTTTTCGCCCTCCCAGACTTCATCTTCCTGTCGGCCCACCAC
GACGAGCGCCTCAACGCCACCCACTGCCAATACAACTTCCCACAGGTGGGCCGCACGGCTCTGC
GGGTGCTGCAGCTGGTGGCTGGCTTTCTGCTGCCCCTGCTGGTCATGGCCTACTGCTATGCCCA
CATCCTGGCCGTGCTGCTGGTTTCCAGGGGCCAGCGGCGCCTGCGGGCCATGCGGCTGGTGGTG
GTGGTCGTGGTGGCCTTTGCCCTCTGCTGGACCCCCTATCACCTGGTGGTGCTGGTGGACATCC
TCATGGACCTGGGCGCTTTGGCCCGCAACTGTGGCCGAGAAAGCAGGGTAGACGTGGCCAAGTC
GGTCACCTCAGGCCTGGGCTACATGCACTGCTGCCTCAACCCGCTGCTCTATGCCTTTGTAGGG
GTCAAGTTCCGGGAGCGGATGTGGATGCTGCTCTTGCGCCTGGGCTGCCCCAACCAGAGAGGGC
TCCAGAGGCAGCCATCGTCTTCCCGCCGGGATTCATCCTGGTCTGAGACCTCAGAGGCCTCCTA
CTCGGGCTTGTGAATTTGGCTACAGCAACAGGGTGGTGGACCTCATGGCCCACATGGCCTCCAA
GGAGTAAGACCCCTGGACCACCAGCCCCAGCAAGAGCACAAGAGGAAGAGAGAGACCCTCACTG
CTGGGGAGTCCCTGCCACACTCAGTCCCCCACCACACTGAATCTCCCCTCCTCACAGTTGCCAT
GTAGACCCCTTGAAGAGGGGAGGGGCCTAGGGAGCCGCACCTTGTCATGTACCATCAATAAAGT
ACCCTGTGCTCAACCAGTTACTTGTCCTGTCTTATTCTAGGGTCTGGGGCAGAGGGGAGGGAAG
CTGGGCTTGTGTCAAGGTGAGACATTCTTGCTGGGGAGGGACCTGGTATGTTCTCCTCAGACTG
AGGGTAGGGCCTCCAAACAGCCTTGCTTGCTTCGAGAACCATTTGCTTCCCGCTCAGACGTCTT
GAGTGCTACAGGAAGCTGGCACCACTACTTCAGAGAACAAGGCCTTTTCCTCTCCTCGCTCCAG
T
exemplary donor template for insertion at GAPDH locus
SEQ ID NO: 216
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGTCCTTGAGGTGAGTG
ACCACCAAGTGCTAAATGACGCCGAGGTTGCCGCCCTCCTGGAGAACTTCAGCTCTTCCTATGA
CTATGGAGAAAACGAGAGTGACTCGTGCTGTACCTCCCCGCCCTGCCCACAGGACTTCAGCCTG
AACTTCGACCGGGCCTTCCTGCCAGCCCTCTACAGCCTCCTCTTTCTGCTGGGGCTGCTGGGCA
ACGGCGCGGTGGCAGCCGTGCTGCTGAGCCGGCGGACAGCCCTGAGCAGCACCGACACCTTCCT
GCTCCACCTAGCTGTAGCAGACACGCTGCTGGTGCTGACACTGCCGCTCTGGGCAGTGGACGCT
GCCGTCCAGTGGGTCTTTGGCTCTGGCCTCTGCAAAGTGGCAGGTGCCCTCTTCAACATCAACT
TCTACGCAGGAGCCCTCCTGCTGGCCTGCATCAGCTTTGACCGCTACCTGAACATAGTTCATGC
CACCCAGCTCTACCGCCGGGGGCCCCCGGCCCGCGTGACCCTCACCTGCCTGGCTGTCTGGGGG
CTCTGCCTGCTTTTCGCCCTCCCAGACTTCATCTTCCTGTCGGCCCACCACGACGAGCGCCTCA
ACGCCACCCACTGCCAATACAACTTCCCACAGGTGGGCCGCACGGCTCTGCGGGTGCTGCAGCT
GGTGGCTGGCTTTCTGCTGCCCCTGCTGGTCATGGCCTACTGCTATGCCCACATCCTGGCCGTG
CTGCTGGTTTCCAGGGGCCAGCGGCGCCTGCGGGCCATGCGGCTGGTGGTGGTGGTCGTGGTGG
CCTTTGCCCTCTGCTGGACCCCCTATCACCTGGTGGTGCTGGTGGACATCCTCATGGACCTGGG
CGCTTTGGCCCGCAACTGTGGCCGAGAAAGCAGGGTAGACGTGGCCAAGTCGGTCACCTCAGGC
CTGGGCTACATGCACTGCTGCCTCAACCCGCTGCTCTATGCCTTTGTAGGGGTCAAGTTCCGGG
AGCGGATGTGGATGCTGCTCTTGCGCCTGGGCTGCCCCAACCAGAGAGGGCTCCAGAGGCAGCC
ATCGTCTTCCCGCCGGGATTCATCCTGGTCTGAGACCTCAGAGGCCTCCTACTCGGGCTTGTGA
ATTTGGCTACAGCAACAGGGTGGTGGACCTCATGGCCCACATGGCCTCCAAGGAGTAAGACCCC
TGGACCACCAGCCCCAGCAAGAGCACAAGAGGAAGAGAGAGACCCTCACTGCTGGGGAGTCCCT
GCCACACTCAGTCCCCCACCACACTGAATCTCCCCTCCTCACAGTTGCCATGTAGACCCCTTGA
AGAGGGGAGGGGCCTAGGGAGCCGCACCTTGTCATGTACCATCAATAAAGTACCCTGTGCTCAA
CCAGTTACTTGTCCTGTCTTATTCTAGGGTCTGGGGCAGAGGGGAGGGAAGCTGGGCTTGTGTC
AAGGTGAGACATTCTTGCTGGGGAGGGACCTGGTATGTTCTCCTCAGACTGAGGGTAGGGCCTC
CAAACAGCCTTGCTTGCTTCGAGAACCATTTGCTTCCCGCTCAGACGTCTTGAGTGCTACAGGA
AGCTGGCACCACTACTTCAGAGAACAAGGCCTTTTCCTCTCCTCGCTCCAGT
exemplary donor template for insertion at GAPDH locus
SEQ ID NO: 217
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGATTATCAAGTGTCAA
GTCCAATCTATGAGATCAATTATTATACATCGGAGCCCTGCCAAAAAATCAATGTGAAGCAAAT
CGCAGCCCGCCTCCTGCCTCCGCTCTACTCACTGGTGTTCATCTTTGGTTTTGTGGGCAACATG
CTGGTCATCCTCATCCTGATAAACTGCAAAAGGCTGAAGAGCATGACTGACATCTACCTGCTCA
ACCTGGCCATCTCTGACCTGTTTTTCCTTCTTACTGTCCCCTTCTGGGCTCACTATGCTGCCGC
CCAGTGGGACTTTGGAAATACAATGTGTCAACTCTTGACAGGGCTCTATTTTATAGGCTTCTTC
TCTGGAATCTTCTTCATCATCCTCCTGACAATCGATAGGTACCTGGCTGTCGTCCATGCTGTGT
TTGCTTTAAAAGCCAGGACGGTCACCTTTGGGGTGGTGACAAGTGTGATCACTTGGGTGGTGGC
TGTGTTTGCGTCTCTCCCAGGAATCATCTTTACCAGATCTCAAAAAGAAGGTCTTCATTACACC
TGGAGCTCTCATTTTCCATACAGTCAGTATCAATTCTGGAAGAATTTCCAGACATTAAAGATAG
TCATCTTGGGGCTGGTCCTGCCGCTGCTTGTCATGGTCATCTGCTACTCGGGAATCCTAAAAAC
TCTGCTTCGGTGTCGAAATGAGAAGAAGAGGCACAGGGCTGTGAGGCTTATCTTCACCATCATG
ATTGTTTATTTTCTCTTCTGGGCTCCCTACAACATTGTCCTTCTCCTGAACACCTTCCAGGAAT
TCTTTGGCCTGAATAATTGCAGTAGCTCTAACAGGTTGGACCAAGCTATGCAGGTGACAGAGAC
TCTTGGGATGACGCACTGCTGCATCAACCCCATCATCTATGCCTTTGTCGGGGAGAAGTTCAGA
AACTACCTCTTAGTCTTCTTCCAAAAGCACATTGCCAAACGCTTCTGCAAATGCTGTTCTATTT
TCCAGCAAGAGGCTCCCGAGCGAGCAAGCTCAGTTTACACCCGATCCACTGGGGAGCAGGAAAT
ATCTGTGGGCTTGTGAATTTGGCTACAGCAACAGGGTGGTGGACCTCATGGCCCACATGGCCTC
CAAGGAGTAAGACCCCTGGACCACCAGCCCCAGCAAGAGCACAAGAGGAAGAGAGAGACCCTCA
CTGCTGGGGAGTCCCTGCCACACTCAGTCCCCCACCACACTGAATCTCCCCTCCTCACAGTTGC
CATGTAGACCCCTTGAAGAGGGGAGGGGCCTAGGGAGCCGCACCTTGTCATGTACCATCAATAA
AGTACCCTGTGCTCAACCAGTTACTTGTCCTGTCTTATTCTAGGGTCTGGGGCAGAGGGGAGGG
AAGCTGGGCTTGTGTCAAGGTGAGACATTCTTGCTGGGGAGGGACCTGGTATGTTCTCCTCAGA
CTGAGGGTAGGGCCTCCAAACAGCCTTGCTTGCTTCGAGAACCATTTGCTTCCCGCTCAGACGT
CTTGAGTGCTACAGGAAGCTGGCACCACTACTTCAGAGAACAAGGCCTTTTCCTCTCCTCGCTC
CAGT
exemplary donor template for insertion at GAPDH locus
SEQ ID NO: 218
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGCTGTCCACATCTCGTT
CTCGGTTTATCAGAAATACCAACGAGAGCGGTGAAGAAGTCACCACCTTTTTTGATTATGATTA
CGGTGCTCCCTGTCATAAATTTGACGTGAAGCAAATTGGGGCCCAACTCCTGCCTCCGCTCTAC
TCGCTGGTGTTCATCTTTGGTTTTGTGGGCAACATGCTGGTCGTCCTCATCTTAATAAACTGCA
AAAAGCTGAAGTGCTTGACTGACATTTACCTGCTCAACCTGGCCATCTCTGATCTGCTTTTTCT
TATTACTCTCCCATTGTGGGCTCACTCTGCTGCAAATGAGTGGGTCTTTGGGAATGCAATGTGC
AAATTATTCACAGGGCTGTATCACATCGGTTATTTTGGCGGAATCTTCTTCATCATCCTCCTGA
CAATCGATAGATACCTGGCTATTGTCCATGCTGTGTTTGCTTTAAAAGCCAGGACGGTCACCTT
TGGGGTGGTGACAAGTGTGATCACCTGGTTGGTGGCTGTGTTTGCTTCTGTCCCAGGAATCATC
TTTACTAAATGCCAGAAAGAAGATTCTGTTTATGTCTGTGGCCCTTATTTTCCACGAGGATGGA
ATAATTTCCACACAATAATGAGGAACATTTTGGGGCTGGTCCTGCCGCTGCTCATCATGGTCAT
CTGCTACTCGGGAATCCTGAAAACCCTGCTTCGGTGTCGAAACGAGAAGAAGAGGCATAGGGCA
GTGAGAGTCATCTTCACCATCATGATTGTTTACTTTCTCTTCTGGACTCCCTATAATATTGTCA
TTCTCCTGAACACCTTCCAGGAATTCTTCGGCCTGAGTAACTGTGAAAGCACCAGTCAACTGGA
CCAAGCCACGCAGGTGACAGAGACTCTTGGGATGACTCACTGCTGCATCAATCCCATCATCTAT
GCCTTCGTTGGGGAGAAGTTCAGAAGCCTTTTTCACATAGCTCTTGGCTGTAGGATTGCCCCAC
TCCAAAAACGAGTGTGTGGAGGTCGAGGAGTGAGACGAGGAAAGAATGTGAAAGTGAGTAGACA
AGGACTCCTCGATGGTCGTGGAAAAGGAAAGTCAATTGGCAGAGCCCCTGAAGCCAGTCTTCAG
GACAAAGAAGGAGCCTAGATTTGGCTACAGCAACAGGGTGGTGGACCTCATGGCCCACATGGCC
TCCAAGGAGTAAGACCCCTGGACCACCAGCCCCAGCAAGAGCACAAGAGGAAGAGAGAGACCCT
CACTGCTGGGGAGTCCCTGCCACACTCAGTCCCCCACCACACTGAATCTCCCCTCCTCACAGTT
GCCATGTAGACCCCTTGAAGAGGGGAGGGGCCTAGGGAGCCGCACCTTGTCATGTACCATCAAT
AAAGTACCCTGTGCTCAACCAGTTACTTGTCCTGTCTTATTCTAGGGTCTGGGGCAGAGGGGAG
GGAAGCTGGGCTTGTGTCAAGGTGAGACATTCTTGCTGGGGAGGGACCTGGTATGTTCTCCTCA
GACTGAGGGTAGGGCCTCCAAACAGCCTTGCTTGCTTCGAGAACCATTTGCTTCCCGCTCAGAC
GTCTTGAGTGCTACAGGAAGCTGGCACCACTACTTCAGAGAACAAGGCCTTTTCCTCTCCTCGC
TCCAGT

### Nuclease

Any nuclease that causes a break within an endogenous coding sequence of an essential gene of the cell can be used in the methods of the present disclosure. In some embodiments the nuclease is a DNA nuclease. In some embodiments the nuclease causes a single-strand break (SSB) within an endogenous coding sequence of an essential gene of the cell, e.g., in a “prime editing” system. In some embodiments the nuclease causes a double-strand break (DSB) within an endogenous coding sequence of an essential gene of the cell. In some embodiments the double-strand break is caused by a single nuclease. In some embodiments the double-strand break is caused by two nucleases that each cause a single-strand break on opposing strands, e.g., a dual “nickase” system. In some embodiments the nuclease is a CRISPR/Cas nuclease and the method further comprises contacting the cell with one or more guide molecules for the CRISPR/Cas nuclease. Exemplary CRISPR/Cas nucleases and guide molecules are described in more detail herein. It is to be understood that the nuclease (including a nickase) is not limited in any manner and can also be a zinc finger nuclease (ZFN), a transcription activator-like effector nuclease (TALEN), a meganuclease, or other nuclease known in the art (or a combination thereof). Methods for designing zinc finger nucleases (ZFNs) are well known in the art, e.g., see Urnov et al., Nature Reviews Genetics 2010; 11:636-640 and Paschon et al., Nat. Commun. 2019; 10(1):1133 and references cited therein. Methods for designing transcription activator-like effector nucleases (TALENs) are well known in the art, e.g., see Joung and Sander, Nat. Rev. Mol. Cell Biol. 2013; 14(1):49-55 and references cited therein. Methods for designing meganucleases are also well known in the art, e.g., see Silva et al., Curr. Gene Ther. 2011; 11(1):11-27 and Redel and Prather, Toxicol. Pathol. 2016; 44(3):428-433.

In some embodiments, a nuclease suitable for methods described herein can have an editing efficiency that is greater than about 50%. In some embodiments, a nuclease suitable for methods described herein can have an editing efficiency that is greater than about 55%. In some embodiments, a nuclease suitable for methods described herein can have an editing efficiency that is greater than about 60%. In some embodiments, a nuclease suitable for methods described herein can have an editing efficiency that is greater than about 65%. In some embodiments, a nuclease suitable for methods described herein can have an editing efficiency that is greater than about 70%. In some embodiments, a nuclease suitable for methods described herein can have an editing efficiency that is greater than about 75%. In some embodiments, a nuclease suitable for methods described herein can have an editing efficiency that is greater than about 80%. In some embodiments, a nuclease suitable for methods described herein can have an editing efficiency that is greater than about 85%. In some embodiments, a nuclease suitable for methods described herein can have an editing efficiency that is greater than about 90%. In some embodiments, a nuclease suitable for methods described herein can have an editing efficiency that is greater than about 95%. In some embodiments, a nuclease suitable for methods described herein can have an editing efficiency that is greater than about 96%. In some embodiments, a nuclease suitable for methods described herein can have an editing efficiency that is greater than about 97%. In some embodiments, a nuclease suitable for methods described herein can have an editing efficiency that is greater than about 98%. In some embodiments, a nuclease suitable for methods described herein can have an editing efficiency that is greater than about 99%.

In general, the nuclease can be delivered to the cell as a protein or a nucleic acid encoding the protein, e.g., a DNA molecule or mRNA molecule. The protein or nucleic acid can be combined with other delivery agents, e.g., lipids or polymers in a lipid or polymer nanoparticle and targeting agents such as antibodies or other binding agents with specificity for the cell. The DNA molecule can be a nucleic acid vector, such as a viral genome or circular double-stranded DNA, e.g., a plasmid. Nucleic acid vectors encoding a nuclease can include other coding or non-coding elements. For example, a nuclease can be delivered as part of a viral genome (e.g., in an AAV, adenoviral or lentiviral genome) that includes certain genomic backbone elements (e.g., inverted terminal repeats, in the case of an AAV genome).

A CRISPR/Cas nuclease can be delivered to the cell as a protein or a nucleic acid encoding the protein, e.g., a DNA molecule or mRNA molecule. The guide molecule can be delivered as an RNA molecule or encoded by a DNA molecule. A CRISPR/Cas nuclease can also be delivered with a guide molecule as a ribonucleoprotein (RNP) and introduced into the cell via nucleofection (electroporation).

CRISPR/Cas Nucleases

CRISPR/Cas nucleases according to the present disclosure include, but are not limited to, naturally-occurring Class 2 CRISPR nucleases such as Cas9, and Cpf1 (Cas12a), as well as other Cas12 nucleases and nucleases derived or obtained therefrom. In functional terms, CRISPR/Cas nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, CRISPR/Cas nucleases can be defined, in broad terms, by their PAM specificity and cleavage activity, even though variations may exist between individual CRISPR/Cas nucleases that share the same PAM specificity or cleavage activity. Skilled artisans will appreciate that some aspects of the present disclosure relate to systems and methods that can be implemented using any suitable CRISPR/Cas nuclease having a certain PAM specificity and/or cleavage activity. For this reason, unless otherwise specified, the term CRISPR/Cas nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity, etc.) of CRISPR/Cas nuclease.

The PAM sequence takes its name from its sequential relationship to the “protospacer” sequence that is complementary to gRNA targeting domains (or “spacers”). Together with protospacer sequences, PAM sequences define target regions or sequences for specific CRISPR/Cas nuclease and gRNA combinations.

Various CRISPR/Cas nucleases may require different sequential relationships between PAMs and protospacers. In general, Cas9s recognize PAM sequences that are 3′ of the protospacer. Cpf1 (Cas12a), on the other hand, generally recognizes PAM sequences that are 5′ of the protospacer.

In addition to recognizing specific sequential orientations of PAMs and protospacers, CRISPR/Cas nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. F. novicida Cpf1 recognizes a TTN PAM sequence. PAM sequences have been identified for a variety of CRISPR/Cas nucleases, and a strategy for identifying novel PAM sequences has been described by Shmakov et al., Molecular Cell 2015; 60:385-397. It should also be noted that engineered CRISPR/Cas nucleases can have PAM specificities that differ from the PAM specificities of reference molecules (for instance, in the case of an engineered CRISPR/Cas nuclease, the reference molecule may be the naturally occurring variant from which the CRISPR/Cas nuclease is derived, or the naturally occurring variant having the greatest amino acid sequence homology to the engineered CRISPR/Cas nuclease).

In addition to their PAM specificity, CRISPR/Cas nucleases can be characterized by their DNA cleavage activity: naturally-occurring CRISPR/Cas nucleases typically form double-strand breaks (DSBs) in target nucleic acids, but engineered variants called “nickases” have been produced that generate only single-strand breaks (SSBs), e.g., those discussed in Ran et al., Cell 2013; 154(6):1380-1389 (“Ran”), or that that do not cut at all.

Cas9

Crystal structures have been determined for S. pyogenes Cas9 (Jinek et al., Science 2014; 343(6176):1247997 (“Jinek 2014”), and for S. aureus Cas9 in complex with a unimolecular guide RNA and a target DNA. See Nishimasu et al., Cell 1024; 156:935-949 (“Nishimasu 2014”); Nishimasu et al., Cell 2015; 162:1113-1126 (“Nishimasu 2015”); and Anders et al., Nature 2014; 513(7519):569-73 (“Anders 2014”).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e., bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e., top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

Cpf1

The crystal structure of Acidaminococcus sp. Cpf1 in complex with crRNA and a dsDNA target including a TTTN PAM sequence has been solved by Yamano et al., Cell. 2016; 165(4):949-962 (“Yamano”). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, —II and —III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Nuclease Variants

The CRISPR/Cas nucleases described herein have activities and properties that can be useful in a variety of applications, but the skilled artisan will appreciate that CRISPR/Cas nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran, Yamano and PCT Publication No. WO 2016/073990A1, the entire contents of each of which are incorporated herein by reference. In general, mutations that reduce or eliminate activity in one of the two nuclease domains result in CRISPR/Cas nucleases with nickase activity, but it should be noted that the type of nickase activity varies depending on which domain is inactivated. As one example, inactivation of a RuvC domain or of a Cas9 HNH domain results in a nickase. Exemplary nickase variants include Cas9 D10A and Cas9 H840A (numbering scheme according to SpCas9 wild-type sequence). Additional suitable nickase variants, including Cas12a variants, will be apparent to the skilled artisan based on the present disclosure and the knowledge in the art. The present disclosure is not limited in this respect. In some embodiments a nickase may be fused to a reverse transcriptase to produce a prime editor (PE), e.g., as described in Anzalone et al., Nature 2019; 576:149-157, the entire contents of which are incorporated herein by reference.

Modifications of PAM specificity relative to naturally occurring Cas9 reference molecules has been described for both S. pyogenes (Kleinstiver et al., Nature 2015; 523(7561):481-5); and S. aureus (Kleinstiver et al., Nat Biotechnol. 2015; 33(12):1293-1298). Modifications that improve the targeting fidelity of Cas9 have also been described (Kleinstiver et al., Nature 2016; 529:490-495). Each of these references is incorporated by reference herein.

CRISPR/Cas nucleases have also been split into two or more parts, as described by Zetsche et al., Nat Biotechnol. 2015; 33(2):139-42, incorporated by reference, and by Fine et al., Sci Rep. 2015; 5:10777, incorporated by reference.

CRISPR/Cas nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotech. 2014; 32:577-582, which is incorporated by reference herein.

CRISPR/Cas nucleases also optionally include a tag, such as, but not limited to, a nuclear localization signal, to facilitate movement of CRISPR/Cas nuclease protein into the nucleus. In certain embodiments, the CRISPR/Cas nuclease can incorporate C- and/or N-terminal nuclear localization signals. Nuclear localization sequences are known in the art.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular CRISPR/Cas nucleases, but it should be understood that the CRISPR/Cas nucleases used may be modified in ways that do not alter their operating principles. Such modifications are within the scope of the present disclosure.

Exemplary suitable nuclease variants include, but are not limited to, AsCpf1 (AsCas12a) variants comprising an M537R substitution, an H800A substitution, and/or an F870L substitution, or any combination thereof (numbering scheme according to AsCpf1 wild-type sequence). In some embodiments, a nuclease variant is a Cas12a variant, e.g., a Cas12a variant comprising 1, 2, or 3 of the amino acid substitutions selected from M537R, F870L, and H800A. In some embodiments, a Cas12a variant comprises an amino acid sequence having at least about 90%, 95%, or 100% identity to an AsCpf1 sequence described herein.

Other suitable modifications of the AsCpf1 amino acid sequence are known to those of ordinary skill in the art. Some exemplary sequences of wild-type AsCpf1 and AsCpf1 variants are provided below:

-His-AsCpf1-sNLS-sNLS H800A amino acid sequence
SEQ ID NO: 58
MGHHHHHHGSTQFEGFTNLYQVSKTLRFELIPQGKTLKHIQEQGFIEEDKARNDHYKELKPIID
RIYKTYADQCLQLVQLDWENLSAAIDSYRKEKTEETRNALIEEQATYRNAIHDYFIGRTDNLTD
AINKRHAEIYKGLFKAELFNGKVLKQLGTVTTTEHENALLRSFDKFTTYFSGFYENRKNVFSAE
DISTAIPHRIVQDNFPKFKENCHIETRLITAVPSLREHFENVKKAIGIEVSTSIEEVFSFPEYN
QLLTQTQIDLYNQLLGGISREAGTEKIKGLNEVLNLAIQKNDETAHIIASLPHRFIPLFKQILS
DRNTLSFILEEFKSDEEVIQSFCKYKTLLRNENVLETAEALFNELNSIDLTHIFISHKKLETIS
SALCDHWDTLRNALYERRISELTGKITKSAKEKVQRSLKHEDINLQEIISAAGKELSEAFKQKT
SEILSHAHAALDQPLPTTLKKQEEKEILKSQLDSLLGLYHLLDWFAVDESNEVDPEFSARLTGI
KLEMEPSLSFYNKARNYATKKPYSVEKFKLNFQMPTLASGWDVNKEKNNGAILFVKNGLYYLGI
MPKQKGRYKALSFEPTEKTSEGFDKMYYDYFPDAAKMIPKCSTQLKAVTAHFQTHTTPILLSNN
FIEPLEITKEIYDLNNPEKEPKKFQTAYAKKTGDQKGYREALCKWIDFTRDFLSKYTKTTSIDL
SSLRPSSQYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAARLGEKMLNKKLKDQKTPIPDTLY
QELYDYVNHRLSHDLSDEARALLPNVITKEVSHEIIKDRRFTSDKFFFHVPITLNYQAANSPSK
FNQRVNAYLKEHPETPIIGIDRGERNLIYITVIDSTGKILEQRSLNTIQQFDYQKKLDNREKER
VAARQAWSVVGTIKDLKQGYLSQVIHEIVDLMIHYQAVVVLENLNFGFKSKRTGIAEKAVYQQF
EKMLIDKLNCLVLKDYPAEKVGGVLNPYQLTDQFTSFAKMGTQSGFLFYVPAPYTSKIDPLTGF
VDPFVWKTIKNHESRKHFLEGFDFLHYDVKTGDFILHFKMNRNLSFQRGLPGFMPAWDIVFEKN
ETQFDAKGTPFIAGKRIVPVIENHRFTGRYRDLYPANELIALLEEKGIVFRDGSNILPKLLEND
DSHAIDTMVALIRSVLQMRNSNAATGEDYINSPVRDLNGVCFDSRFQNPEWPMDADANGAYHIA
LKGQLLLNHLKESKDLKLQNGISNQDWLAYIQELRNGSPKKKRKVGSPKKKRKV
-Cpf1 variant 1 amino acid sequence
SEQ ID NO: 59
MTQFEGFTNLYQVSKTLRFELIPQGKTLKHIQEQGFIEEDKARNDHYKELKPIIDRIYKTYADQ
CLQLVQLDWENLSAAIDSYRKEKTEETRNALIEEQATYRNAIHDYFIGRTDNLTDAINKRHAEI
YKGLFKAELFNGKVLKQLGTVTTTEHENALLRSFDKFTTYFSGFYENRKNVFSAEDISTAIPHR
IVQDNFPKFKENCHIFTRLITAVPSLREHFENVKKAIGIFVSTSIEEVFSFPFYNQLLTQTQID
LYNQLLGGISREAGTEKIKGLNEVLNLAIQKNDETAHIIASLPHRFIPLFKQILSDRNTLSFIL
EEFKSDEEVIQSFCKYKTLLRNENVLETAEALFNELNSIDLTHIFISHKKLETISSALCDHWDT
LRNALYERRISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIKLEMEPSLS
FYNKARNYATKKPYSVEKFKLNFQRPTLASGWDVNKEKNNGAILFVKNGLYYLGIMPKQKGRYK
ALSFEPTEKTSEGFDKMYYDYFPDAAKMIPKCSTQLKAVTAHFQTHTTPILLSNNFIEPLEITK
EIYDLNNPEKEPKKFQTAYAKKTGDQKGYREALCKWIDFTRDFLSKYTKTTSIDLSSLRPSSQY
KDLGEYYAELNPLLYHISFQRIAEKEIMDAVETGKLYLFQIYNKDFAKGHHGKPNLHTLYWTGL
FSPENLAKTSIKLNGQAELFYRPKSRMKRMAHRLGEKMLNKKLKDQKTPIPDTLYQELYDYVNH
RLSHDLSDEARALLPNVITKEVSHEIIKDRRFTSDKFLFHVPITLNYQAANSPSKFNQRVNAYL
KEHPETPIIGIDRGERNLIYITVIDSTGKILEQRSLNTIQQFDYQKKLDNREKERVAARQAWSV
VGTIKDLKQGYLSQVIHEIVDLMIHYQAVVVLENLNFGFKSKRTGIAEKAVYQQFEKMLIDKLN
CLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGT
PFIAGKRIVPVIENHRFTGRYRDLYPANELIALLEEKGIVFRDGSNILPKLLENDDSHAIDTMV
ALIRSVLQMRNSNAATGEDYINSPVRDLNGVCFDSRFQNPEWPMDADANGAYHIALKGQLLLNH
LKESKDLKLQNGISNQDWLAYIQELRNGRSSDDEATADSQHAAPPKKKRKVGGSGGSGGSGGSG
GSGGSGGSGGSLEHHHHHH
-Cpf1 variant 2 amino acid sequence
SEQ ID NO: 60
MTQFEGFTNLYQVSKTLRFELIPQGKTLKHIQEQGFIEEDKARNDHYKELKPIIDRIYKTYADQ
CLQLVQLDWENLSAAIDSYRKEKTEETRNALIEEQATYRNAIHDYFIGRTDNLTDAINKRHAEI
YKGLFKAELFNGKVLKQLGTVTTTEHENALLRSFDKETTYFSGEYENRKNVFSAEDISTAIPHR
IVQDNFPKFKENCHIFTRLITAVPSLREHFENVKKAIGIFVSTSIEEVFSFPFYNQLLTQTQID
LYNQLLGGISREAGTEKIKGLNEVLNLAIQKNDETAHIIASLPHRFIPLFKQILSDRNTLSFIL
EEFKSDEEVIQSFCKYKTLLRNENVLETAEALFNELNSIDLTHIFISHKKLETISSALCDHWDT
LRNALYERRISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIKLEMEPSLS
FYNKARNYATKKPYSVEKFKLNFQMPTLASGWDVNKEKNNGAILFVKNGLYYLGIMPKQKGRYK
ALSFEPTEKTSEGFDKMYYDYFPDAAKMIPKCSTQLKAVTAHFQTHTTPILLSNNFIEPLEITK
EIYDLNNPEKEPKKFQTAYAKKTGDQKGYREALCKWIDFTRDFLSKYTKTTSIDLSSLRPSSQY
KDLGEYYAELNPLLYHISFQRIAEKEIMDAVETGKLYLFQIYNKDFAKGHHGKPNLHTLYWTGL
FSPENLAKTSIKLNGQAELFYRPKSRMKRMAHRLGEKMLNKKLKDQKTPIPDTLYQELYDYVNH
RLSHDLSDEARALLPNVITKEVSHEIIKDRRFTSDKFFFHVPITLNYQAANSPSKFNQRVNAYL
KEHPETPIIGIDRGERNLIYITVIDSTGKILEQRSLNTIQQFDYQKKLDNREKERVAARQAWSV
VGTIKDLKQGYLSQVIHEIVDLMIHYQAVVVLENLNFGFKSKRTGIAEKAVYQQFEKMLIDKLN
CLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGT
PFIAGKRIVPVIENHRFTGRYRDLYPANELIALLEEKGIVFRDGSNILPKLLENDDSHAIDTMV
ALIRSVLQMRNSNAATGEDYINSPVRDLNGVCFDSRFQNPEWPMDADANGAYHIALKGQLLLNH
LKESKDLKLQNGISNQDWLAYIQELRNGRSSDDEATADSQHAAPPKKKRKVGGSGGSGGSGGSG
GSGGSGGSGGSLEHHHHHH
-Cpf1 variant 3 amino acid sequence
SEQ ID NO: 61
MTQFEGFTNLYQVSKTLRFELIPQGKTLKHIQEQGFIEEDKARNDHYKELKPIIDRIYKTYADQ
CLQLVQLDWENLSAAIDSYRKEKTEETRNALIEEQATYRNAIHDYFIGRTDNLTDAINKRHAEI
YKGLFKAELFNGKVLKQLGTVTTTEHENALLRSFDKFTTYFSGFYENRKNVFSAEDISTAIPHR
IVQDNFPKFKENCHIFTRLITAVPSLREHFENVKKAIGIFVSTSIEEVFSFPFYNQLLTQTQID
LYNQLLGGISREAGTEKIKGLNEVLNLAIQKNDETAHIIASLPHRFIPLFKQILSDRNTLSFIL
EEFKSDEEVIQSFCKYKTLLRNENVLETAEALFNELNSIDLTHIFISHKKLETISSALCDHWDT
LRNALYERRISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIKLEMEPSLS
FYNKARNYATKKPYSVEKFKLNFQRPTLASGWDVNKEKNNGAILFVKNGLYYLGIMPKQKGRYK
ALSFEPTEKTSEGFDKMYYDYFPDAAKMIPKCSTQLKAVTAHFQTHTTPILLSNNFIEPLEITK
EIYDLNNPEKEPKKFQTAYAKKTGDQKGYREALCKWIDFTRDFLSKYTKTTSIDLSSLRPSSQY
KDLGEYYAELNPLLYHISFQRIAEKEIMDAVETGKLYLFQIYNKDFAKGHHGKPNLHTLYWTGL
FSPENLAKTSIKLNGQAELFYRPKSRMKRMAARLGEKMLNKKLKDQKTPIPDTLYQELYDYVNH
RLSHDLSDEARALLPNVITKEVSHEIIKDRRFTSDKFLFHVPITLNYQAANSPSKFNQRVNAYL
KEHPETPIIGIDRGERNLIYITVIDSTGKILEQRSLNTIQQFDYQKKLDNREKERVAARQAWSV
VGTIKDLKQGYLSQVIHEIVDLMIHYQAVVVLENLNFGFKSKRTGIAEKAVYQQFEKMLIDKLN
CLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGT
PFIAGKRIVPVIENHRFTGRYRDLYPANELIALLEEKGIVFRDGSNILPKLLENDDSHAIDTMV
ALIRSVLQMRNSNAATGEDYINSPVRDLNGVCFDSRFQNPEWPMDADANGAYHIALKGQLLLNH
LKESKDLKLQNGISNQDWLAYIQELRNGRSSDDEATADSQHAAPPKKKRKVGGSGGSGGSGGSG
GSGGSGGSGGSLEHHHHHH
-Cpf1 variant 4 amino acid sequence
SEQ ID NO: 62
MTQFEGFTNLYQVSKTLRFELIPQGKTLKHIQEQGFIEEDKARNDHYKELKPIIDRIYKTYADQ
CLQLVQLDWENLSAAIDSYRKEKTEETRNALIEEQATYRNAIHDYFIGRTDNLTDAINKRHAEI
YKGLFKAELFNGKVLKQLGTVTTTEHENALLRSFDKFTTYFSGFYENRKNVFSAEDISTAIPHR
IVQDNFPKFKENCHIFTRLITAVPSLREHFENVKKAIGIFVSTSIEEVFSFPFYNQLLTQTQID
LYNQLLGGISREAGTEKIKGLNEVLNLAIQKNDETAHIIASLPHRFIPLFKQILSDRNTLSFIL
EEFKSDEEVIQSFCKYKTLLRNENVLETAEALFNELNSIDLTHIFISHKKLETISSALCDHWDT
LRNALYERRISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIKLEMEPSLS
FYNKARNYATKKPYSVEKFKLNFQRPTLASGWDVNKEKNNGAILFVKNGLYYLGIMPKQKGRYK
ALSFEPTEKTSEGFDKMYYDYFPDAAKMIPKCSTQLKAVTAHFQTHTTPILLSNNFIEPLEITK
EIYDLNNPEKEPKKFQTAYAKKTGDQKGYREALCKWIDFTRDFLSKYTKTTSIDLSSLRPSSQY
KDLGEYYAELNPLLYHISFQRIAEKEIMDAVETGKLYLFQIYNKDFAKGHHGKPNLHTLYWTGL
FSPENLAKTSIKLNGQAELFYRPKSRMKRMAARLGEKMLNKKLKDQKTPIPDTLYQELYDYVNH
RLSHDLSDEARALLPNVITKEVSHEIIKDRRFTSDKFLFHVPITLNYQAANSPSKFNQRVNAYL
KEHPETPIIGIDRGERNLIYITVIDSTGKILEQRSLNTIQQFDYQKKLDNREKERVAARQAWSV
VGTIKDLKQGYLSQVIHEIVDLMIHYQAVVVLENLNFGFKSKRTGIAEKAVYQQFEKMLIDKLN
CLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGT
PFIAGKRIVPVIENHRFTGRYRDLYPANELIALLEEKGIVFRDGSNILPKLLENDDSHAIDTMV
ALIRSVLQMRNSNAATGEDYINSPVRDLNGVCFDSRFQNPEWPMDADANGAYHIALKGQLLLNH
LKESKDLKLQNGISNQDWLAYIQELRNGRSSDDEATADSQHAAPPKKKRKV
-Cpf1 variant 5 amino acid sequence
SEQ ID NO: 63
MTQFEGFTNLYQVSKTLRFELIPQGKTLKHIQEQGFIEEDKARNDHYKELKPIIDRIYKTYADQ
CLQLVQLDWENLSAAIDSYRKEKTEETRNALIEEQATYRNAIHDYFIGRTDNLTDAINKRHAEI
YKGLFKAELFNGKVLKQLGTVTTTEHENALLRSFDKFTTYFSGFYENRKNVFSAEDISTAIPHR
IVQDNFPKFKENCHIFTRLITAVPSLREHFENVKKAIGIFVSTSIEEVFSFPFYNQLLTQTQID
LYNQLLGGISREAGTEKIKGLNEVLNLAIQKNDETAHIIASLPHRFIPLFKQILSDRNTLSFIL
EEFKSDEEVIQSFCKYKTLLRNENVLETAEALFNELNSIDLTHIFISHKKLETISSALCDHWDT
LRNALYERRISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIKLEMEPSLS
FYNKARNYATKKPYSVEKFKLNFQRPTLASGWDVNKEKNNGAILFVKNGLYYLGIMPKQKGRYK
ALSFEPTEKTSEGFDKMYYDYFPDAAKMIPKCSTQLKAVTAHFQTHTTPILLSNNFIEPLEITK
EIYDLNNPEKEPKKFQTAYAKKTGDQKGYREALCKWIDFTRDFLSKYTKTTSIDLSSLRPSSQY
KDLGEYYAELNPLLYHISFQRIAEKEIMDAVETGKLYLFQIYNKDFAKGHHGKPNLHTLYWTGL
FSPENLAKTSIKLNGQAELFYRPKSRMKRMAHRLGEKMLNKKLKDQKTPIPDTLYQELYDYVNH
RLSHDLSDEARALLPNVITKEVSHEIIKDRRFTSDKFLFHVPITLNYQAANSPSKFNQRVNAYL
KEHPETPIIGIDRGERNLIYITVIDSTGKILEQRSLNTIQQFDYQKKLDNREKERVAARQAWSV
VGTIKDLKQGYLSQVIHEIVDLMIHYQAVVVLENLNFGFKSKRTGIAEKAVYQQFEKMLIDKLN
CLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGT
PFIAGKRIVPVIENHRFTGRYRDLYPANELIALLEEKGIVFRDGSNILPKLLENDDSHAIDTMV
ALIRSVLQMRNSNAATGEDYINSPVRDLNGVCFDSRFQNPEWPMDADANGAYHIALKGQLLLNH
LKESKDLKLQNGISNQDWLAYIQELRNGRSSDDEATADSQHAAPPKKKRKV
-Cpf1 variant 6 amino acid sequence
SEQ ID NO: 64
MTQFEGFTNLYQVSKTLRFELIPQGKTLKHIQEQGFIEEDKARNDHYKELKPIIDRIYKTYADQ
CLQLVQLDWENLSAAIDSYRKEKTEETRNALIEEQATYRNAIHDYFIGRTDNLTDAINKRHAEI
YKGLFKAELFNGKVLKQLGTVTTTEHENALLRSFDKFTTYFSGFYENRKNVFSAEDISTAIPHR
IVQDNFPKFKENCHIFTRLITAVPSLREHFENVKKAIGIFVSTSIEEVFSFPFYNQLLTQTQID
LYNQLLGGISREAGTEKIKGLNEVLNLAIQKNDETAHIIASLPHRFIPLFKQILSDRNTLSFIL
EEFKSDEEVIQSFCKYKTLLRNENVLETAEALFNELNSIDLTHIFISHKKLETISSALCDHWDT
LRNALYERRISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIKLEMEPSLS
FYNKARNYATKKPYSVEKFKLNFQRPTLASGWDVNKEKNNGAILFVKNGLYYLGIMPKQKGRYK
ALSFEPTEKTSEGFDKMYYDYFPDAAKMIPKCSTQLKAVTAHFQTHTTPILLSNNFIEPLEITK
EIYDLNNPEKEPKKFQTAYAKKTGDQKGYREALCKWIDFTRDFLSKYTKTTSIDLSSLRPSSQY
KDLGEYYAELNPLLYHISFQRIAEKEIMDAVETGKLYLFQIYNKDFAKGHHGKPNLHTLYWTGL
FSPENLAKTSIKLNGQAELFYRPKSRMKRMAHRLGEKMLNKKLKDQKTPIPDTLYQELYDYVNH
RLSHDLSDEARALLPNVITKEVSHEIIKDRRFTSDKFLFHVPITLNYQAANSPSKFNQRVNAYL
KEHPETPIIGIDRGERNLIYITVIDSTGKILEQRSLNTIQQFDYQKKLDNREKERVAARQAWSV
VGTIKDLKQGYLSQVIHEIVDLMIHYQAVVVLENLNFGFKSKRTGIAEKAVYQQFEKMLIDKLN
CLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGT
PFIAGKRIVPVIENHRFTGRYRDLYPANELIALLEEKGIVFRDGSNILPKLLENDDSHAIDTMV
ALIRSVLQMRNSNAATGEDYINSPVRDLNGVCFDSRFQNPEWPMDADANGAYHIALKGQLLLNH
LKESKDLKLQNGISNQDWLAYIQELRNGRSSDDEATADSQHAAPPKKKRKVGGSGGSGGSGGSG
GSGGSGGSGGSLEHHHHHH
-Cpf1 variant 7 amino acid sequence
SEQ ID NO: 65
MGRDPGKPIPNPLLGLDSTAPKKKRKVGIHGVPAATQFEGFTNLYQVSKTLRFELIPQGKTLKH
IQEQGFIEEDKARNDHYKELKPIIDRIYKTYADQCLQLVQLDWENLSAAIDSYRKEKTEETRNA
LIEEQATYRNAIHDYFIGRTDNLTDAINKRHAEIYKGLFKAELFNGKVLKQLGTVTTTEHENAL
LRSFDKFTTYFSGFYENRKNVFSAEDISTAIPHRIVQDNFPKFKENCHIFTRLITAVPSLREHF
ENVKKAIGIFVSTSIEEVFSFPFYNQLLTQTQIDLYNQLLGGISREAGTEKIKGLNEVLNLAIQ
KNDETAHIIASLPHRFIPLFKQILSDRNTLSFILEEFKSDEEVIQSFCKYKTLLRNENVLETAE
ALFNELNSIDLTHIFISHKKLETISSALCDHWDTLRNALYERRISELTGKITKSAKEKVQRSLK
HEDINLQEIISAAGKELSEAFKQKTSEILSHAHAALDQPLPTTLKKQEEKEILKSQLDSLLGLY
HLLDWFAVDESNEVDPEFSARLTGIKLEMEPSLSFYNKARNYATKKPYSVEKFKLNFQMPTLAS
GWDVNKEKNNGAILFVKNGLYYLGIMPKQKGRYKALSFEPTEKTSEGFDKMYYDYFPDAAKMIP
KCSTQLKAVTAHFQTHTTPILLSNNFIEPLEITKEIYDLNNPEKEPKKFQTAYAKKTGDQKGYR
EALCKWIDFTRDFLSKYTKTTSIDLSSLRPSSQYKDLGEYYAELNPLLYHISFQRIAEKEIMDA
VETGKLYLFQIYNKDFAKGHHGKPNLHTLYWTGLFSPENLAKTSIKLNGQAELFYRPKSRMKRM
AHRLGEKMLNKKLKDQKTPIPDTLYQELYDYVNHRLSHDLSDEARALLPNVITKEVSHEIIKDR
RFTSDKFFFHVPITLNYQAANSPSKFNQRVNAYLKEHPETPIIGIDRGERNLIYITVIDSTGKI
LEQRSLNTIQQFDYQKKLDNREKERVAARQAWSVVGTIKDLKQGYLSQVIHEIVDLMIHYQAVV
VLENLNFGFKSKRTGIAEKAVYQQFEKMLIDKLNCLVLKDYPAEKVGGVLNPYQLTDQFTSFAK
MGTQSGFLFYVPAPYTSKIDPLTGFVDPFVWKTIKNHESRKHFLEGFDFLHYDVKTGDFILHFK
MNRNLSFQRGLPGFMPAWDIVFEKNETQFDAKGTPFIAGKRIVPVIENHRFTGRYRDLYPANEL
IALLEEKGIVFRDGSNILPKLLENDDSHAIDTMVALIRSVLQMRNSNAATGEDYINSPVRDLNG
VCFDSRFQNPEWPMDADANGAYHIALKGQLLLNHLKESKDLKLQNGISNQDWLAYIQELRNPKK
KRKVKLAAALEHHHHHH
-Exemplary AsCpf1 wild-type amino acid sequence
SEQ ID NO: 66
MTQFEGFTNLYQVSKTLRFELIPQGKTLKHIQEQGFIEEDKARNDHYKELKPIIDRIYKTYADQ
CLQLVQLDWENLSAAIDSYRKEKTEETRNALIEEQATYRNAIHDYFIGRTDNLTDAINKRHAEI
YKGLFKAELFNGKVLKQLGTVTTTEHENALLRSFDKFTTYFSGFYENRKNVFSAEDISTAIPHR
IVQDNFPKFKENCHIFTRLITAVPSLREHFENVKKAIGIFVSTSIEEVFSFPFYNQLLTQTQID
LYNQLLGGISREAGTEKIKGLNEVLNLAIQKNDETAHIIASLPHRFIPLFKQILSDRNTLSFIL
EEFKSDEEVIQSFCKYKTLLRNENVLETAEALFNELNSIDLTHIFISHKKLETISSALCDHWDT
LRNALYERRISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIKLEMEPSLS
FYNKARNYATKKPYSVEKFKLNFQMPTLASGWDVNKEKNNGAILFVKNGLYYLGIMPKQKGRYK
ALSFEPTEKTSEGFDKMYYDYFPDAAKMIPKCSTQLKAVTAHFQTHTTPILLSNNFIEPLEITK
EIYDLNNPEKEPKKFQTAYAKKTGDQKGYREALCKWIDFTRDFLSKYTKTTSIDLSSLRPSSQY
KDLGEYYAELNPLLYHISFQRIAEKEIMDAVETGKLYLFQIYNKDFAKGHHGKPNLHTLYWTGL
FSPENLAKTSIKLNGQAELFYRPKSRMKRMAHRLGEKMLNKKLKDQKTPIPDTLYQELYDYVNH
RLSHDLSDEARALLPNVITKEVSHEIIKDRRFTSDKFFFHVPITLNYQAANSPSKFNQRVNAYL
KEHPETPIIGIDRGERNLIYITVIDSTGKILEQRSLNTIQQFDYQKKLDNREKERVAARQAWSV
VGTIKDLKQGYLSQVIHEIVDLMIHYQAVVVLENLNFGFKSKRTGIAEKAVYQQFEKMLIDKLN
CLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGT
PFIAGKRIVPVIENHRFTGRYRDLYPANELIALLEEKGIVFRDGSNILPKLLENDDSHAIDTMV
ALIRSVLQMRNSNAATGEDYINSPVRDLNGVCFDSRFQNPEWPMDADANGAYHIALKGQLLLNH
LKESKDLKLQNGISNQDWLAYIQELRN

Additional suitable nucleases and nuclease variants will be apparent to the skilled artisan based on the present disclosure in view of the knowledge in the art. Exemplary suitable nucleases may include, but are not limited to those provided in Table 5.

TABLE 5
Exemplary Suitable CRISPR/Cas Nucleases

| | Length | | |
|---|---|---|---|
| Nuclease | (A.A.) | PAM | Reference |
| SpCas9 | 1368 | NGG | Cong et al., Science 2013; 339(6121): 819-23 |
| SaCas9 | 1053 | NNGRRT | Ran et al., Nature 2015; 520(7546): 186-91. |
| (KKH) | 1067 | NNNRRT | Kleinstiver et al., Nat Biotechnol. 2015; |
| SaCas9 | | | 33(12): 1293-1298 |
| AsCpf1 | 1353 | TTTV | Zetsche et al., Nat Biotechnol. 2017; 35(1): 31- |
| (AsCas12a) | | | 34. |
| LbCpf1 | 1274 | TTTV | Zetsche et al., Cell 2015; 163(3): 759-71. |
| (LbCas12a) | | | |
| CasX | 980 | TTC | Burstein et al., Nature 2017; 542(7640): 237- |
| | | | 241. |
| CasY | 1200 | TA | Burstein et al., Nature 2017; 542(7640): 237- |
| | | | 241. |
| Cas12h1 | 870 | RTR | Yan et al., Science 2019; 363(6422): 88-91. |
| Cas12i1 | 1093 | TTN | Yan et al., Science 2019; 363(6422): 88-91. |
| Cas12c1 | unknown | TG | Yan et al., Science 2019; 363(6422): 88-91. |
| Cas12c2 | unknown | TN | Yan et al., Science 2019; 363(6422): 88-91. |
| eSpCas9 | 1423 | NGG | Chen et al., Nature 2017; 550(7676): 407-410. |
| Cas9-HF1 | 1367 | NGG | Chen et al., Nature 2017; 550(7676): 407-410. |
| HypaCas9 | 1404 | NGG | Chen et al., Nature 2017; 550(7676): 407-410. |
| dCas9-Fokl | 1623 | NGG | U.S. Patent No. 9,322,037 |
| Sniper-Cas9 | 1389 | NGG | Lee et al., Nat Commun. 2018; 9(1): 3048. |
| xCas9 | 1786 | NGG, NG, | Hu et al., Nature. 2018 Apr 5;556(7699): 57-63. |
| | | GAA, GAT | |
| AaCas12b | 1129 | TTN | Teng et al., Cell Discov. 2018; 4: 63. |
| evoCas9 | 1423 | NGG | Casini et al., Nat Biotechnol. 2018; 36(3): 265- |
| | | | 271. |
| SpCas9-NG | 1423 | NG | Nishimasu et al., Science 2018; |
| | | | 361(6408): 1259-1262. |
| VRQR | 1368 | NGA | Li et al., The CRISPR Journal, 2018; 01: 01 |
| VRER | 1372 | NGCG | Kleinstiver et al., Nature 2016; 529(7587): 490- |
| | | | 5. |
| NmeCas9 | 1082 | NNNNGATT | Amrani et al., Genome Biol. 2018; 19(1): 214. |
| CjCas9 | 984 | NNNNRYAC | Kim et al., Nat Commun. 2017; 8: 14500. |
| BhCas12b | 1108 | ATTN | Strecker et al., Nat Commun. 2019; 10(1): 212. |
| BhCas12b V4 | 1108 | ATTN | Pausch et al., Science 2020; 369(6501): 333- |
| | | | 337. |

Guide RNA (gRNA) Molecules

Guide RNAs (gRNAs) of the present disclosure may be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al., Molecular Cell 2014; 56(2):333-339 (“Briner”), and in PCT Publication No. WO2016/073990A1.

In bacteria and archaea, type II CRISPR systems generally comprise an CRISPR/Cas nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). See Mali et al., Science 2013; 339(6121):823-826 (“Mali”); Jiang et al., Nat Biotechnol. 2013; 31(3):233-239 (“Jiang”); and Jinek et al., Science 2012; 337(6096):816-821 (“Jinek 2012”).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013; 31(9):827-832, (“Hsu”)), “complementarity regions” (PCT Publication No. WO2016/073990A1), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. See Nishimasu 2014 and 2015. It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. See Nishimasu 2015. A first stem-loop one near the 3′ portion of the second complementarity domain is referred to variously as the “proximal domain,” (PCT Publication No. WO2016/073990A1) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other CRISPR/Cas nucleases have been (or may in the future be) discovered or invented which utilize gRNAs that differ in some ways from those described to this point. For instance, Cpf1 (“CRISPR from Prevotella and Franciscella 1”) which is also called Cas12a is a CRISPR/Cas nuclease that does not require a tracrRNA to function (see Zetsche et al., Cell 2015; 163:759-771 (“Zetsche I”)). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple CRISPR/Cas nucleases. For this reason, unless otherwise specified, the term gRNA should be understood to encompass any suitable gRNA that can be used with any CRISPR/Cas nuclease, and not only those gRNAs that are compatible with a particular species of Cas9 or Cpf1. By way of illustration, the term gRNA can, in certain embodiments, include a gRNA for use with any CRISPR/Cas nuclease occurring in a Class 2 CRISPR system, such as a type II or type V or CRISPR system, or an CRISPR/Cas nuclease derived or adapted therefrom.

In some embodiments a method or system of the present disclosure may use more than one gRNA. In some embodiments, two or more gRNAs may be used to create two or more double strand breaks in the genome of a cell. In some embodiments, a multiplexed editing strategy may be used that targets two or more essential genes at the same time with two or more knock-in cassettes. In some such embodiments, the two or more knock-in cassettes may comprise different exogenous cargo sequences, e.g., different knock-in cassettes may encode different gene products of interest and thus the edited cells will express a plurality of gene products of interest from different knock-in cassettes targeted to different loci.

In some embodiments using more than one gRNA, a double-strand break may be caused by a dual-gRNA paired “nickase” strategy. In some embodiments for selecting gRNAs, including the determination for which gRNAs can be used for the dual-gRNA paired “nickase” strategy, gRNA pairs should be oriented on the DNA such that PAMs are facing out and cutting with the D10A Cas9 nickase will result in 5′ overhangs.

In some embodiments, a method or system of the present disclosure may use a prime editing gRNA (pegRNA) in conjunction with a prime editor (PE). As is well known in the art, a pegRNA is substantially larger than standard gRNAs, e.g., in some embodiments longer than 50, 100, 150 or 250 nucleotides, e.g., as described in Anzalone et al., Nature 2019; 576:17-19-157, the entire contents of which are incorporated herein by reference. The pegRNA is a gRNA with a primer binding sequence (PBS) and a donor template containing the desired RNA sequence added at one of the termini, e.g., the 3′ end. The PE:pegRNA complex binds to the target DNA, and the nickase domain of the prime editor nicks only one strand, generating a flap. The PBS, located on the pegRNA, binds to the DNA flap and the edited RNA sequence is reverse transcribed using the reverse transcriptase domain of the prime editor. The edited strand is incorporated into the DNA at the end of the nicked flap, and the target DNA is repaired with the new reverse transcribed DNA. The original DNA segment is removed by a cellular endonuclease. This leaves one strand edited, and one strand unedited. In the newest PE systems, e.g., PE3 and PE3b, the unedited strand can be corrected to match the newly edited strand by using an additional standard gRNA. In this case, the unedited strand is nicked by a nickase and the newly edited strand is used as a template to repair the nick, thus completing the edit.

gRNA Design

Methods for selection and validation of target sequences as well as off-target analyses have been described previously, e.g., in Mali; Hsu; Fu et al., Nat Biotechnol 2014; 32(3):279-84, Heigwer et al., Nat methods 2014; 11(2):122-3; Bae et al., Bioinformatics 2014; 30(10):1473-5; and Xiao et al. Bioinformatics 2014; 30(8):1180-1182. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in PCT Publication No. WO2016/073990A1.

For example, methods for selection and validation of target sequences as well as off-target analyses can be performed using cas-offinder (Bae et al., Bioinformatics 2014; 30:1473-5). Cas-offender is a tool that can quickly identify all sequences in a genome that have up to a specified number of mismatches to a guide sequence.

As another example, methods for scoring how likely a given sequence is to be an off-target (e.g., once candidate target sequences are identified) can be performed. An exemplary score includes a Cutting Frequency Determination (CFD) score, as described by Doench et al., Nat Biotechnol. 2016; 34:184-91.

gRNA Modifications

In certain embodiments, gRNAs as used herein may be modified or unmodified gRNAs. In certain embodiments, a gRNA may include one or more modifications. In certain embodiments, the one or more modifications may include a phosphorothioate linkage modification, a phosphorodithioate (PS2) linkage modification, a 2′-O-methyl modification, or combinations thereof. In certain embodiments, the one or more modifications may be at the 5′ end of the gRNA, at the 3′ end of the gRNA, or combinations thereof.

In certain embodiments, a gRNA modification may comprise one or more phosphorodithioate (PS2) linkage modifications.

In some embodiments, a gRNA used herein includes one or more or a stretch of deoxyribonucleic acid (DNA) bases, also referred to herein as a “DNA extension.” In some embodiments, a gRNA used herein includes a DNA extension at the 5′ end of the gRNA, the 3′ end of the gRNA, or a combination thereof. In certain embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, or 25 DNA bases long. In certain embodiments, the DNA extension may include one or more DNA bases selected from adenine (A), guanine (G), cytosine (C), or thymine (T). In certain embodiments, the DNA extension includes the same DNA bases. For example, the DNA extension may include a stretch of adenine (A) bases. In certain embodiments, the DNA extension may include a stretch of thymine (T) bases. In certain embodiments, the DNA extension includes a combination of different DNA bases.

Exemplary suitable 5′ extensions for Cpf1 guide RNAs are provided in Table 6 below:

TABLE 6
Exemplary Cpf1 gRNA 5′ Extensions

| SEQ | | 5′ |
|---|---|---|
| ID NO: | 5′ extension sequence | modification |
| N/A | rCrUrUrUrU | +5 RNA |
| 67 | rArArGrArCrCrUrUrUrU | +10 RNA |
| 68 | TArUrGrUrGrUrUrUrUrUrGrUrCrArArArArGrArCr | +25 RNA |
| | CrUrUrUrU | |
| 69 | TArGrGrCrCrArGrCrUrUrGrCrCrGrGrUrUrUrUrUr | +60 RNA |
| | UrArGrUrCrGrUrGrCrUrGrCrUrUrCrArUrGrUrGr | |
| | UrUrUrUrUrGrUrCrArArArArGrArCrCrUrUrUrU | |
| N/A | CTTTT | +5 DNA |
| 70 | AAGACCTTTT | +10 DNA |
| 71 | ATGTGTTTTTGTCAAAAGACCTTTT | +25 DNA |
| 72 | AGGCCAGCTTGCCGGTTTTTTAGTCGTGCTGC | +60 DNA |
| | TTCATGTGTTTTTGTCAAAAGACCTTTT | |
| 73 | TTTTTGTCAAAAGACCTTTT | +20 DNA |
| 74 | GCTTCATGTGTTTTTGTCAAAAGACCTTTT | +30 DNA |
| 75 | GCCGGTTTTTTAGTCGTGCTGCTTCATGTGTT | +50 DNA |
| | TTTGTCAAAAGACCTTTT | |
| 76 | TAGTCGTGCTGCTTCATGTGTTTTTGTCAAAA | +40 DNA |
| | GACCTTTT | |
| 77 | C*C*GAAGTTTTCTTCGGTTTT | +20 DNA + |
| | | 2xPS |
| 78 | T*T*TTTCCGAAGTTTTCTTCGGTTTT | +25 DNA + |
| | | 2xPS |
| 79 | A*A*CGCTTTTTCCGAAGTTTTCTTCGGTTTT | +30 DNA + |
| | | 2xPS |
| 80 | G*C*GTTGTTTTCAACGCTTTTTCCGAAGTTTT | +41 DNA + |
| | CTTCGGTTTT | 2xPS |
| 81 | G*G*CTTCTTTTGAAGCCTTTTTGCGTTGTTTT | +62 DNA + |
| | CAACGCTTTTTCCGAAGTTTTCTTCGGTTTT | 2xPS |
| 82 | A*T*GTGTTTTTGTCAAAAGACCTTTT | +25 DNA + |
| | | 2xPS |
| 83 | AAAAAAAAAAAAAAAAAAAAAAAAA | +25 A |
| 84 | TTTTTTTTTTTTTTTTTTTTTTTTT | +25 T |
| 85 | mA*mU*rGrUrGrUrUrUrUrUrGrUrCrArArArArGr | +25 RNA + |
| | ArCrCrUrUrUrU | 2xPS |
| 86 | mA*mA*rArArArArArArArArArArArArArArArAr | PolyA RNA + |
| | ArArArArArArA | 2xPS |
| 87 | mU*mU*rUrUrUrUrUrUrUrUrUrUrUrUrUrUrUrUr | PolyU RNA + |
| | UrUrUrUrUrUrU | 2xPS |

In certain embodiments, a gRNA used herein includes a DNA extension as well as a chemical modification, e.g., one or more phosphorothioate linkage modifications, one or more phosphorodithioate (PS2) linkage modifications, one or more 2′-O-methyl modifications, or one or more additional suitable chemical gRNA modification disclosed herein, or combinations thereof. In certain embodiments, the one or more modifications may be at the 5′ end of the gRNA, at the 3′ end of the gRNA, or combinations thereof.

Without wishing to be bound by theory, it is contemplated that any DNA extension may be used with any gRNA disclosed herein, so long as it does not hybridize to the target nucleic acid being targeted by the gRNA and it also exhibits an increase in editing at the target nucleic acid site relative to a gRNA which does not include such a DNA extension.

In some embodiments, a gRNA used herein includes one or more or a stretch of ribonucleic acid (RNA) bases, also referred to herein as an “RNA extension.” In some embodiments, a gRNA used herein includes an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may include one or more RNA bases selected from adenine (rA), guanine (rG), cytosine (rC), or uracil (rU), in which the “r” represents RNA, 2′-hydroxy. In certain embodiments, the RNA extension includes the same RNA bases. For example, the RNA extension may include a stretch of adenine (rA) bases. In certain embodiments, the RNA extension includes a combination of different RNA bases. In certain embodiments, a gRNA used herein includes an RNA extension as well as one or more phosphorothioate linkage modifications, one or more phosphorodithioate (PS2) linkage modifications, one or more 2′-O-methyl modifications, one or more additional suitable gRNA modification, e.g., chemical modification, disclosed herein, or combinations thereof. In certain embodiments, the one or more modifications may be at the 5′ end of the gRNA, at the 3′ end of the gRNA, or combinations thereof. In certain embodiments, a gRNA including a RNA extension may comprise a sequence set forth herein.

It is contemplated that gRNAs used herein may also include an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

In some embodiments, a gRNA which includes a modification, e.g., a DNA extension at the 5′ end and/or a chemical modification as disclosed herein, is complexed with a CRISPR/Cas nuclease, e.g., an AsCpf1 nuclease, to form an RNP, which is then employed to edit a target cell, e.g., a pluripotent stem cell or a progeny thereof.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)), as shown below:

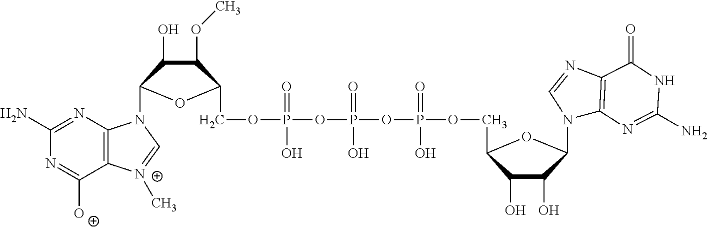

The cap or cap analog can be included during either chemical or enzymatic synthesis of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical or enzymatic synthesis, using a polyadenosine polymerase (e.g., E. coli Poly(A)Polymerase).

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

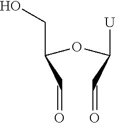

wherein “U” can be an unmodified or modified uridine.

The 3′ terminal U ribose can be modified with a 2′3′ cyclic phosphate as shown below:

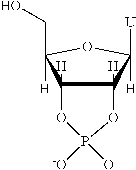

wherein “U” can be an unmodified or modified uridine.

Guide RNAs can contain 3′ nucleotides that can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into a gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂, alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, including without limitation methylene, propylene, ether, or amino bridges; O-amino (wherein amino can be, e.g., NH₂, alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂, alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into a gRNA. In certain embodiments, O- and N-alkylated nucleotides, e.g., N6-methyl adenosine, can be incorporated into a gRNA. In certain embodiments, one or more or all of the nucleotides in a gRNA are deoxynucleotides.

Guide RNAs can also include one or more cross-links between complementary regions of the crRNA (at its 3′ end) and the tracrRNA (at its 5′ end) (e.g., within a “tetraloop” structure and/or positioned in any stem loop structure occurring within a gRNA). A variety of linkers are suitable for use. For example, guide RNAs can include common linking moieties including, without limitation, polyvinylether, polyethylene, polypropylene, polyethylene glycol (PEG), polyvinyl alcohol (PVA), polyglycolide (PGA), polylactide (PLA), polycaprolactone (PCL), and copolymers thereof.

In some embodiments, a bifunctional cross-linker is used to link a 5′ end of a first gRNA fragment and a 3′ end of a second gRNA fragment, and the 3′ or 5′ ends of the gRNA fragments to be linked are modified with functional groups that react with the reactive groups of the cross-linker. In general, these modifications comprise one or more of amine, sulfhydryl, carboxyl, hydroxyl, alkene (e.g., a terminal alkene), azide and/or another suitable functional group. Multifunctional (e.g. bifunctional) cross-linkers are also generally known in the art, and may be either heterofunctional or homofunctional, and may include any suitable functional group, including without limitation isothiocyanate, isocyanate, acyl azide, an NHS ester, sulfonyl chloride, tosyl ester, tresyl ester, aldehyde, amine, epoxide, carbonate (e.g., Bis(p-nitrophenyl) carbonate), aryl halide, alkyl halide, imido ester, carboxylate, alkyl phosphate, anhydride, fluorophenyl ester, HOBt ester, hydroxymethyl phosphine, O-methylisourea, DSC, NHS carbamate, glutaraldehyde, activated double bond, cyclic hemiacetal, NHS carbonate, imidazole carbamate, acyl imidazole, methylpyridinium ether, azlactone, cyanate ester, cyclic imidocarbonate, chlorotriazine, dehydroazepine, 6-sulfo-cytosine derivatives, maleimide, aziridine, TNB thiol, Ellman's reagent, peroxide, vinylsulfone, phenylthioester, diazoalkanes, diazoacetyl, epoxide, diazonium, benzophenone, anthraquinone, diazo derivatives, diazirine derivatives, psoralen derivatives, alkene, phenyl boronic acid, etc. In some embodiments, a first gRNA fragment comprises a first reactive group and the second gRNA fragment comprises a second reactive group. For example, the first and second reactive groups can each comprise an amine moiety, which are crosslinked with a carbonate-containing bifunctional crosslinking reagent to form a urea linkage. In other instances, (a) the first reactive group comprises a bromoacetyl moiety and the second reactive group comprises a sulfhydryl moiety, or (b) the first reactive group comprises a sulfhydryl moiety and the second reactive group comprises a bromoacetyl moiety, which are crosslinked by reacting the bromoacetyl moiety with the sulfhydryl moiety to form a bromoacetyl-thiol linkage. These and other cross-linking chemistries are known in the art, and are summarized in the literature, including by Greg T. Hermanson, Bioconjugate Techniques, 3rd Ed. 2013, published by Academic Press.

Additional suitable gRNA modifications will be apparent to those of ordinary skill in the art based on the present disclosure. Suitable gRNA modifications include, for example, those described in PCT Publication No. WO2019070762A1 entitled “MODIFIED CPF1 GUIDE RNA;” in PCT Publication No. WO2016089433A1 entitled “GUIDE RNA WITH CHEMICAL MODIFICATIONS;” in PCT Publication No. WO2016164356A1 entitled “CHEMICALLY MODIFIED GUIDE RNAS FOR CRISPR/CAS-MEDIATED GENE REGULATION;” and in PCT Publication No. WO2017053729A1 entitled “NUCLEASE-MEDIATED GENOME EDITING OF PRIMARY CELLS AND ENRICHMENT THEREOF;” the entire contents of each of which are incorporated herein by reference.

Exemplary gRNAs

Non-limiting examples of guide RNAs suitable for certain embodiments embraced by the present disclosure are provided herein, for example, in the Tables below. Those of ordinary skill in the art will be able to envision suitable guide RNA sequences for a specific nuclease, e.g., a Cas9 or Cpf1 nuclease, from the disclosure of the targeting domain sequence, either as a DNA or RNA sequence. For example, a guide RNA comprising a targeting sequence consisting of RNA nucleotides would include the RNA sequence corresponding to the targeting domain sequence provided as a DNA sequence, and this contain uracil instead of thymidine nucleotides. For example, a guide RNA comprising a targeting domain sequence consisting of RNA nucleotides, and described by the DNA sequence TCTGCAGAAATGTTCCCCGT (SEQ ID NO: 88) would have a targeting domain of the corresponding RNA sequence UCUGCAGAAAUGUUCCCCGU (SEQ ID NO: 89). As will be apparent to the skilled artisan, such a targeting sequence would be linked to a suitable guide RNA scaffold, e.g., a crRNA scaffold sequence or a chimeric crRNA/tracrRNA scaffold sequence. Suitable gRNA scaffold sequences are known to those of ordinary skill in the art. For AsCpf1, for example, a suitable scaffold sequence comprises the sequence UAAUUUCUACUCUUGUAGAU (SEQ ID NO: 90), added to the 5′-terminus of the targeting domain. In the example above, this would result in a Cpf1 guide RNA of the sequence UAAUUUCUACUCUUGUAGAUUCUGCAGAAAUGUUCCCCGU (SEQ ID NO: 91). Those of skill in the art would further understand how to modify such a guide RNA, e.g., by adding a DNA extension (e.g., in the example above, adding a 25-mer DNA extension as described herein would result, for example, in a guide RNA of the sequence ATGTGTTTTTGTCAAAAGACCTTTTrUrArArUrUrUrCrUrArCrUrCrUrUrGrUrArGrArUrUr CrUrGrCrArGrArArArUrGrUrUrCrCrCrCrGrU (SEQ ID NO: 92)). It will be understood that the exemplary targeting sequences provided herein are not limiting, and additional suitable sequences, e.g., variants of the specific sequences disclosed herein, will be apparent to the skilled artisan based on the present disclosure in view of the general knowledge in the art.

It will be understood that the exemplary gRNAs disclosed herein are provided to illustrate non-limiting embodiments embraced by the present disclosure. Additional suitable gRNA sequences will be apparent to the skilled artisan based on the present disclosure, and the disclosure is not limited in this respect.

### Target Cells

Methods of the disclosure can be used to edit the genome of any cell. In certain embodiments, the target cell is a stem cell, e.g., an iPS or ES cell. In certain embodiments, the target cell can be an iPS- or ES-derived cell, where the genetic modification is made at any stage during the reprogramming process from donor cell to iPSC, during the iPSC stage, and/or at any stage of the process of differentiating the iPSC or ESC to a specialized cell, or even up to or at the final specialized cell state. In certain embodiments, the target cell can be an iPS-derived NK cell (iNK cell) or iPS-derived T cell (iT cell) where the genetic modification is made at any stage during the reprogramming process from donor cell to iPSC, during the iPSC stage, and/or at any stage of the process of differentiating the iPSC to an iNK or iT state, e.g., at an intermediary state, such as, for example, an iPSC-derived HSC state, or even up to or at the final iNK or iT cell state.

In certain embodiments, a target cell is one or more of a long-term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a fibroblast, a monocyte-derived macrophage or dendritic cell, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte. In some embodiments, a target cell is a neuronal progenitor cell. In some embodiments, a target cell is a neuron.

In some embodiments, a target cell is a circulating blood cell, e.g., a reticulocyte, megakaryocyte erythroid progenitor (MEP) cell, myeloid progenitor cell (CMP/GMP), lymphoid progenitor (LP) cell, hematopoietic stem/progenitor cell (HSC), or endothelial cell (EC). In some embodiments, a target cell is one or more of a bone marrow cell (e.g., a reticulocyte, an erythroid cell (e.g., erythroblast), an MEP cell, myeloid progenitor cell (CMP/GMP), LP cell, erythroid progenitor (EP) cell, HSC, multipotent progenitor (MPP) cell, endothelial cell (EC), hemogenic endothelial (HE) cell, or mesenchymal stem cell). In some embodiments, a target cell is one or more of a myeloid progenitor cell (e.g., a common myeloid progenitor (CMP) cell or granulocyte macrophage progenitor (GMP) cell). In some embodiments, a target cell is a lymphoid progenitor cell, e.g., a common lymphoid progenitor (CLP) cell. In some embodiments, a target cell is one or more of an erythroid progenitor cell (e.g., an MEP cell). In some embodiments, a target cell is one or more of a hematopoietic stem/progenitor cell (e.g., a long term HSC (LT-HSC), short term HSC (ST-HSC), MPP cell, or lineage restricted progenitor (LRP) cell). In certain embodiments, the target cell is a CD34⁺ cell, CD34⁺CD90⁺ cell, CD34⁺CD38⁻ cell, CD34⁺CD90⁺CD49f⁺CD38⁻CD45RA⁻ cell, CD105⁺ cell, CD31⁺, or CD133⁺ cell, or a CD34⁺CD90⁺ CD133⁺ cell. In some embodiments, a target cell is one or more of an umbilical cord blood CD34⁺ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In some embodiments, a target cell is one or more of a mobilized peripheral blood hematopoietic CD34⁺ cell (after the subject is treated with a mobilization agent, e.g., G-CSF or Plerixafor). In some embodiments, a target cell is a peripheral blood endothelial cell. In some embodiments, a target cell is a peripheral blood natural killer cell.

In certain embodiments, a target cell is a primary cell, e.g., a cell isolated from a human subject. In certain embodiments, a target cell is an immune cell, e.g., a primary immune cell isolated from a human subject. In certain embodiments, a target cell is part of a population of cells isolated from a subject, e.g., a human subject. In some embodiments, the population of cells comprises a population of immune cells isolated from a subject. In some embodiments, the population of cells comprises tumor infiltrating lymphocytes (TILs), e.g., TILs isolated from a human subject. In some embodiments, a target cell is isolated from a healthy subject, e.g., a healthy human donor. In some embodiments, a target cell is isolated from a subject having a disease or illness, e.g., a human patient in need of a treatment.

In certain embodiments, a target cell is an immune cell, e.g., a primary immune cell, e.g., a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4⁺ naïve T cell, a TH17 CD4⁺ T cell, a TH1 CD4⁺ T cell, a TH2 CD4⁺ T cell, a TH9 CD4⁺ T cell, a CD4⁺Foxp3⁺ T cell, a CD4⁺CD25⁺ CD127⁻ T cell, or a CD4⁺CD25⁺ CD127⁻ Foxp3⁺ T cell. In some embodiments, a target cell is an alpha-beta T cell, a gamma-delta T cell or a Treg. In some embodiments a target cell is macrophage. In some embodiments, a target cell is an innate lymphoid cell. In some embodiments, a target cell is a dendritic cell. In some embodiments, a target cell is a beta cell, e.g., a pancreatic beta cell.

In some embodiments, a target cell is isolated from a subject having a cancer.

In some embodiments, a target cell is isolated from a subject having a cancer, including but not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma, medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer); hematopoietic cancer (e.g., lymphomas, primary pulmonary lymphomas, bronchus-associated lymphoid tissue lymphomas, splenic lymphomas, nodal marginal zone lymphomas, pediatric B cell non-Hodgkin lymphomas); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); melanoma; midline tract carcinoma; multiple endocrine neoplasia syndrome; muscle cancer; mesothelioma; nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); parathyroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (WE), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; vulvar cancer (e.g., Paget's disease of the vulva), or any combination thereof.

In some embodiments, a target cell is isolated from a subject having a hematological disorder. In some embodiments, a target cell is isolated form a subject having sickle cell anemia. In some embodiments, a target cell is isolated from a subject having β-thalassemia.

### Stem Cells

Methods of the disclosure can be used with stem cells. Stem cells are typically cells that have the capacity to produce unaltered daughter cells (self-renewal; cell division produces at least one daughter cell that is identical to the parent cell) and to give rise to specialized cell types (potency). Stem cells include, but are not limited to, embryonic stem (ES) cells, embryonic germ (EG) cells, germline stem (GS) cells, human mesenchymal stem cells (hMSCs), adipose tissue-derived stem cells (ADSCs), multipotent adult progenitor cells (MAPCs), multipotent adult germline stem cells (maGSCs) and unrestricted somatic stem cell (USSCs). Generally, stem cells can divide without limit. After division, the stem cell may remain as a stem cell, become a precursor cell, or proceed to terminal differentiation. A precursor cell is a cell that can generate a fully differentiated functional cell of at least one given cell type. Generally, precursor cells can divide. After division, a precursor cell can remain a precursor cell, or may proceed to terminal differentiation.

Pluripotent stem cells are generally known in the art. The present disclosure provides technologies (e.g., systems, compositions, methods, etc.) related to pluripotent stem cells. In some embodiments, pluripotent stem cells are stem cells that: (a) are capable of inducing teratomas when transplanted in immunodeficient (SCID) mice; (b) are capable of differentiating to cell types of all three germ layers (e.g., can differentiate to ectodermal, mesodermal, and endodermal cell types); and/or (c) express one or more markers of embryonic stem cells (e.g., human embryonic stem cells express Oct-4, alkaline phosphatase, SSEA-3 surface antigen, SSEA-4 surface antigen, nanog, TRA-1-60, TRA-1-81, Sox-2, REX1, etc.). In some aspects, human pluripotent stem cells do not show expression of differentiation markers. In some embodiments, ES cells and/or iPSCs edited using methods of the disclosure maintain their pluripotency, e.g., (a) are capable of inducing teratomas when transplanted in immunodeficient (SCID) mice; (b) are capable of differentiating to cell types of all three germ layers, e.g., can differentiate to ectodermal, mesodermal, and endodermal cell types); and/or (c) express one or more markers of embryonic stem cells.

In some embodiments, ES cells (e.g., human ES cells) can be derived from the inner cell mass of blastocysts or morulae. In some embodiments, ES cells can be isolated from one or more blastomeres of an embryo, e.g., without destroying the remainder of the embryo. In some embodiments, ES cells can be produced by somatic cell nuclear transfer. In some embodiments, ES cells can be derived from fertilization of an egg cell with sperm or DNA, nuclear transfer, parthenogenesis, or by means to generate ES cells, e.g., with homozygosity in the HLA region. In some embodiments, human ES cells can be produced or derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce an embryonic cell. Exemplary human ES cells are known in the art and include, but are not limited to, MAO1, MAO9, ACT-4, No. 3, H1, H7, H9, H14 and ACT30 ES cells. In some embodiments, human ES cells, regardless of their source or the particular method used to produce them, can be identified based on, e.g., (i) the ability to differentiate into cells of all three germ layers, (ii) expression of at least Oct-4 and alkaline phosphatase, and/or (iii) ability to produce teratomas when transplanted into immunocompromised animals. In some embodiments, ES cells have been serially passaged as cell lines.

iPS Cells

Induced pluripotent stem cells (iPSC) are a type of pluripotent stem cell artificially derived from a non-pluripotent cell, such as an adult somatic cell (e.g., a fibroblast cell or other suitable somatic cell), by inducing expression of certain genes. iPSCs can be derived from any organism, such as a mammal. In some embodiments, iPSCs are produced from mice, rats, rabbits, guinea pigs, goats, pigs, cows, non-human primates or humans. iPSCs are similar to ES cells in many respects, such as the expression of certain stem cell genes and proteins, chromatin methylation patterns, doubling time, embryoid body formation, teratoma formation, viable chimera formation, potency and/or differentiability. Various suitable methods for producing iPSCs are known in the art. In some embodiments, iPSCs can be derived by transfection of certain stem cell-associated genes (such as Oct-3/4 (Pouf51) and Sox-2) into non-pluripotent cells, such as adult fibroblasts. Transfection can be achieved through viral vectors, such as retroviruses, lentiviruses, or adenoviruses. Additional suitable reprogramming methods include the use of vectors that do not integrate into the genome of the host cell, e.g., episomal vectors, or the delivery of reprogramming factors directly via encoding RNA or as proteins has also been described. For example, cells can be transfected with Oct-3/4, Sox-2, Klf4, and/or c-Myc using a retroviral system or with Oct-4, Sox-2, NANOG, and/or LIN28 using a lentiviral system. After 3-4 weeks, small numbers of transfected cells begin to become morphologically and biochemically similar to pluripotent stem cells, and can be isolated through morphological selection, doubling time, or through a reporter gene and antibiotic selection. In one example, iPSCs from adult human cells are generated by the method described by Yu et al., Science 2007; 318(5854):1224 or Takahashi et al., Cell 2007; 131:861-72. Numerous suitable methods for reprogramming are known to those of skill in the art, and the present disclosure is not limited in this respect.

In some embodiments, a target cell for the editing and cargo integration methods described herein is an iPSC, wherein the edited iPSC is then differentiated, e.g., into an iPSC-derived immune cell. In some embodiments, the differentiated cell is an iPSC-derived immune cell. In some embodiments, the differentiated cell is an iPSC-derived iNK cell, an iPSC-derived T cell (e.g., an iPSC-derived alpha-beta T cell, gamma-delta T cell, Treg, CD4+ T cell, or CD8+ T cell), an iPSC-derived dendritic cell, or an iPSC-derived macrophage. In some embodiments, the differentiated cell is an iPSC-derived pancreatic beta cell.

iNK Cells

In some embodiments, the present disclosure provides methods of generating iNK cells (e.g., genetically modified iNK cells).

In some embodiments, genetic modifications present in an iNK cell of the present disclosure can be made at any stage during the reprogramming process from donor cell to iPSC, during the iPSC stage, and/or at any stage of the process of differentiating the iPSC to an iNK state, e.g., at an intermediary state, such as, for example, an iPSC-derived HSC state, or even up to or at the final iNK cell state.

For example, one or more genomic modifications present in a genetically modified iNK cell of the present disclosure may be made at one or more different cell stages (e.g., reprogramming from donor to iPSC, differentiation of iPSC to iNK). In some embodiments, one or more genomic modifications present in a genetically modified iNK cell provided herein is made before reprogramming a donor cell to an iPSC state. In some embodiments, all edits present in a genetically modified iNK cell provided herein are made at the same time, in close temporal proximity, and/or at the same cell stage of the reprogramming/differentiation process, e.g., at the donor cell stage, during the reprogramming process, at the iPSC stage, or during the differentiation process, e.g., from iPSC to iNK. In some embodiments, two or more edits present in a genetically modified iNK cell provided herein are made at different times and/or at different cell stages of the reprogramming/differentiation process from donor cell to iPSC to iNK. For example, in some embodiments, a first edit is made at the donor cell stage and a second (different) edit is made at the iPSC stage. In some embodiments, a first edit is made at the reprogramming stage (e.g., donor to iPSC) and a second (different) edit is made at the iPSC stage.

A variety of cell types can be used as a donor cell that can be subjected to reprogramming, differentiation, and/or genetic engineering strategies described herein. For example, the donor cell can be a pluripotent stem cell or a differentiated cell, e.g., a somatic cell, such as, for example, a fibroblast or a T lymphocyte. In some embodiments, donor cells are manipulated (e.g., subjected to reprogramming, differentiation, and/or genetic engineering) to generate iNK cells described herein.

A donor cell can be from any suitable organism. For example, in some embodiments, the donor cell is a mammalian cell, e.g., a human cell or a non-human primate cell. In some embodiments, the donor cell is a somatic cell. In some embodiments, the donor cell is a stem cell or progenitor cell. In certain embodiments, the donor cell is not or was not part of a human embryo and its derivation does not involve destruction of a human embryo.

In some embodiments, a genetically modified iNK cell is derived from an iPSC, which in turn is derived from a somatic donor cell. Any suitable somatic cell can be used in the generation of iPSCs, and in turn, the generation of iNK cells. Suitable strategies for deriving iPSCs from various somatic donor cell types have been described and are known in the art. In some embodiments, a somatic donor cell is a fibroblast cell. In some embodiments, a somatic donor cell is a mature T cell.

For example, in some embodiments, a somatic donor cell, from which an iPSC, and subsequently an iNK cell is derived, is a developmentally mature T cell (a T cell that has undergone thymic selection). One hallmark of developmentally mature T cells is a rearranged T cell receptor locus. During T cell maturation, the TCR locus undergoes V(D)J rearrangements to generate complete V-domain exons. These rearrangements are retained throughout reprogramming of a T cells to an iPSC, and throughout differentiation of the resulting iPSC to a somatic cell.

In certain embodiments, a somatic donor cell is a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4⁺ naïve T cell, a TH17 CD4⁺ T cell, a TH1 CD4⁺ T cell, a TH2 CD4⁺ T cell, a TH9 CD4⁺ T cell, a CD4⁺Foxp3⁺ T cell, a CD4⁺CD25⁺ CD127⁻ T cell, or a CD4⁺CD25⁺ CD127⁻ Foxp3⁺ T cell.

T cells can be advantageous for the generation of iPSCs. For example, T cells can be edited with relative ease, e.g., by CRISPR-based methods or other genetic engineering methods. Additionally, the rearranged TCR locus allows for genetic tracking of individual cells and their daughter cells. For example, if the reprogramming, expansion, culture, and/or differentiation strategies involved in the generation of NK cells a clonal expansion of a single cell, the rearranged TCR locus can be used as a genetic marker unambiguously identifying a cell and its daughter cells. This, in turn, allows for the characterization of a cell population as truly clonal, or for the identification of mixed populations, or contaminating cells in a clonal population. Another potential advantage of using T cells in generating iNK cells carrying multiple edits is that certain karyotypic aberrations associated with chromosomal translocations are selected against in T cell culture. Such aberrations can pose a concern when editing cells by CRISPR technology, and in particular when generating cells carrying multiple edits. Using T cell derived iPSCs as a starting point for the derivation of therapeutic lymphocytes can allow for the expression of a pre-screened TCR in the lymphocytes, e.g., via selecting the T cells for binding activity against a specific antigen, e.g., a tumor antigen, reprogramming the selected T cells to iPSCs, and then deriving lymphocytes from these iPSCs that express the TCR (e.g., T cells). This strategy can allow for activating the TCR in other cell types, e.g., by genetic or epigenetic strategies. Additionally, T cells retain at least part of their “epigenetic memory” throughout the reprogramming process, and thus subsequent differentiation of the same or a closely related cell type, such as iNK cells can be more efficient and/or result in higher quality cell populations as compared to approaches using non-related cells, such as fibroblasts, as a starting point for iNK derivation.

In some embodiments, a donor cell being manipulated, e.g., a cell being reprogrammed and/or undergoing genetic engineering as described herein, is one or more of a long-term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a fibroblast, a monocyte-derived macrophage or dendritic cell, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte.

In some embodiments, a donor cell is one or more of a circulating blood cell, e.g., a reticulocyte, megakaryocyte erythroid progenitor (MEP) cell, myeloid progenitor cell (CMP/GMP), lymphoid progenitor (LP) cell, hematopoietic stem/progenitor cell (HSC), or endothelial cell (EC). In some embodiments, a donor cell is one or more of a bone marrow cell (e.g., a reticulocyte, an erythroid cell (e.g., erythroblast), an MEP cell, myeloid progenitor cell (CMP/GMP), LP cell, erythroid progenitor (EP) cell, HSC, multipotent progenitor (MPP) cell, endothelial cell (EC), hemogenic endothelial (HE) cell, or mesenchymal stem cell). In some embodiments, a donor cell is one or more of a myeloid progenitor cell (e.g., a common myeloid progenitor (CMP) cell or granulocyte macrophage progenitor (GMP) cell). In some embodiments, a donor cell is one or more of a lymphoid progenitor cell, e.g., a common lymphoid progenitor (CLP) cell. In some embodiments, a donor cell is one or more of an erythroid progenitor cell (e.g., an MEP cell). In some embodiments, a donor cell is one or more of a hematopoietic stem/progenitor cell (e.g., a long term HSC (LT-HSC), short term HSC (ST-HSC), MPP cell, or lineage restricted progenitor (LRP) cell). In certain embodiments, the donor cell is a CD34⁺ cell, CD34⁺CD90⁺ cell, CD34⁺CD38⁻ cell, CD34⁺CD90⁺CD49f⁺CD38⁻CD45RA⁻ cell, CD105⁺ cell, CD31⁺, or CD133⁺ cell, or a CD34⁺CD90⁺ CD133⁺ cell. In some embodiments, a donor cell is one or more of an umbilical cord blood CD34⁺ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In some embodiments, a donor cell is one or more of a mobilized peripheral blood hematopoietic CD34⁺ cell (after the subject is treated with a mobilization agent, e.g., G-CSF or Plerixafor). In some embodiments, a donor cell is a peripheral blood endothelial cell. In some embodiments, a donor cell is a peripheral blood natural killer cell.

In some embodiments, a donor cell is a dividing cell. In some embodiments, a donor cell is a non-dividing cell.

In some embodiments, a genetically modified (e.g., edited) iNK cell resulting from one or more methods and/or strategies described herein, are administered to a subject in need thereof, e.g., in the context of an immuno-oncology therapeutic approach. In some embodiments, donor cells, or any cells of any stage of the reprogramming, differentiating, and/or genetic engineering strategies provided herein, can be maintained in culture or stored (e.g., frozen in liquid nitrogen) using any suitable method known in the art, e.g., for subsequent characterization or administration to a subject in need thereof.

### Methods of Characterization

Methods of characterizing cells including characterizing cellular phenotype are known to those of skill in the art. In some embodiments, one or more such methods may include, but not be limited to, for example, morphological analyses and flow cytometry. Cellular lineage and identity markers are known to those of skill in the art. One or more such markers may be combined with one or more characterization methods to determine a composition of a cell population or phenotypic identity of one or more cells. For example, in some embodiments, cells of a particular population will be characterized using flow cytometry (for example, see Ye Li et al., Cell Stem Cell. 2018 Aug. 2; 23(2): 181-192.e5). In some such embodiments, a sample of a population of cells will be evaluated for presence and proportion of one or more cell surface markers and/or one or more intracellular markers. As will be understood by those of skill in the art, such cell surface markers may be representative of different lineages. For example, pluripotent cells may be identified by one or more of any number of markers known to be associated with such cells, such as, for example, CD34. Further, in some embodiments, cells may be identified by markers that indicate some degree of differentiation. Such markers will be known to one of skill in the art. For example, in some embodiments, markers of differentiated cells may include those associated with differentiated hematopoietic cells such as, e.g., CD43, CD45 (differentiated hematopoietic cells). In some embodiments, markers of differentiated cells may be associated with NK cell phenotypes such as, e.g., CD56, NK cell receptor immunoglobulin gamma Fc region receptor III (FcγRIII, cluster of differentiation 16 (CD16)), natural killer group-2 member D (NKG2D), CD69, a natural cytotoxicity receptor, etc. In some embodiments, markers may be T cell markers (e.g., CD3, CD4, CD8, etc.).

### Methods of Use

A variety of diseases, disorders and/or conditions may be treated through use of cells provided by the present disclosure. For example, in some embodiments, a disease, disorder and/or condition may be treated by introducing genetically modified or engineered cells as described herein (e.g., genetically modified iNK cells) to a subject. Examples of diseases that may be treated include, but are not limited to, cancer, e.g., solid tumors, e.g., of the brain, prostate, breast, lung, colon, uterus, skin, liver, bone, pancreas, ovary, testes, bladder, kidney, head, neck, stomach, cervix, rectum, larynx, or esophagus; and hematological malignancies, e.g., acute and chronic leukemias, lymphomas, multiple myeloma and myelodysplastic syndromes.

In some embodiments, the present disclosure provides methods of treating a subject in need thereof by administering to the subject a composition comprising any of the cells described herein. In some embodiments, a therapeutic agent or composition may be administered before, during, or after the onset of a disease, disorder, or condition (including, e.g., an injury). In some embodiments, the present disclosure provides any of the cells described herein for use in the preparation of a medicament. In some embodiments, the present disclosure provides any of the cells described herein for use in the treatment of a disease, disorder, or condition, that can be treated by a cell therapy.

In particular embodiments, the subject has a disease, disorder, or condition, that can be treated by a cell therapy. In some embodiments, a subject in need of cell therapy is a subject with a disease, disorder and/or condition, whereby a cell therapy, e.g., a therapy in which a composition comprising a cell described herein, is administered to the subject, whereby the cell therapy treats at least one symptom associated with the disease, disorder, and/or condition. In some embodiments, a subject in need of cell therapy includes, but is not limited to, a candidate for bone marrow or stem cell transplant, a subject who has received chemotherapy or irradiation therapy, a subject who has or is at risk of having cancer, e.g., a cancer of hematopoietic system, a subject having or at risk of developing a tumor, e.g., a solid tumor, and/or a subject who has or is at risk of having a viral infection or a disease associated with a viral infection.

Pharmaceutical Compositions

In some embodiments, the present disclosure provides pharmaceutical compositions comprising one or more genetically modified or engineered cells described herein, e.g., a genetically modified iNK cell described herein. In some embodiments, a pharmaceutical composition further comprises a pharmaceutically acceptable excipient. In some embodiments, a pharmaceutical composition comprises isolated pluripotent stem cell-derived hematopoietic lineage cells comprising at least 50%, 60%, 70%, 80%, 90%, 95%, 98%, or 99% T cells, NK cells, NKT cells, CD34+HE cells or HSCs, e.g., genetically modified (e.g., edited) T cells, NK cells, NKT cells, CD34+HE cells or HSCs. In some embodiments, a pharmaceutical composition comprises isolated pluripotent stem cell-derived hematopoietic lineage cells comprising about 95% to about 100% T cells, NK cells, NKT cells, CD34+HE cells or HSCs, e.g., genetically modified (e.g., edited) T cells, NK cells, NKT cells, CD34+HE cells or HSCs.

In some embodiments, a pharmaceutical composition of the present disclosure comprises an isolated population of pluripotent stem cell-derived hematopoietic lineage cells, wherein the isolated population has less than about 0.1%, 0.5%, 1%, 2%, 5%, 10%, 15%, 20%, 25%, or 30% T cells, NK cells, NKT cells, CD34+HE cells or HSCs, e.g., genetically modified (e.g., edited) T cells, NK cells, NKT cells, CD34+HE cells or HSCs. In some embodiments, an isolated population of pluripotent stem cell-derived hematopoietic lineage cells has more than about 0.1%, 0.5%, 1%, 2%, 5%, 10%, 15%, 20%, 25%, or 30% T cells, NK cells, NKT cells, CD34+HE cells or HSCs, e.g., genetically modified (e.g., edited) T cells, NK cells, NKT cells, CD34+HE cells or HSCs. In some embodiments, an isolated population of pluripotent stem cell-derived hematopoietic lineage cells has about 0.1% to about 1%, about 1% to about 3%, about 3% to about 5%, about 10%-15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% T cells, NK cells, NKT cells, CD34+HE cells or HSCs, e.g., genetically modified (e.g., edited) T cells, NK cells, NKT cells, CD34+HE cells or HSCs.

In some embodiments, an isolated population of pluripotent stem cell-derived hematopoietic lineage cells comprises about 0.1%, about 1%, about 3%, about 5%, about 10%, about 15%, about 20%, about 25%, about 30%, about 35%, about 40%, about 45%, about 50%, about 60%, about 70%, about 80%, about 90%, about 95%, about 98%, about 99%, or about 100% T cells, NK cells, NKT cells, CD34+HE cells or HSCs, e.g., genetically modified (e.g., edited) T cells, NK cells, NKT cells, CD34+HE cells or HSCs.

As one of ordinary skill in the art would understand, both autologous and allogeneic cells can be used in adoptive cell therapies. Autologous cell therapies generally have reduced infection, low probability for GVHD, and rapid immune reconstitution relative to other cell therapies. Allogeneic cell therapies generally have an immune mediated graft-versus-malignancy (GVM) effect, and low rate of relapse relative to other cell therapies. Based on the specific condition(s) of the subject in need of the cell therapy, one of ordinary skill in the art would be able to determine which specific type of therapy(ies) to administer.

In some embodiments, a pharmaceutical composition comprises pluripotent stem cell-derived hematopoietic lineage cells that are allogeneic to a subject. In some embodiments, a pharmaceutical composition comprises pluripotent stem cell-derived hematopoietic lineage cells that are autologous to a subject. For autologous transplantation, the isolated population of pluripotent stem cell-derived hematopoietic lineage cells can be either a complete or partial HLA-match with the subject being treated. In some embodiments, the pluripotent stem cell-derived hematopoietic lineage cells are not HLA-matched to a subject.

In some embodiments, pluripotent stem cell-derived hematopoietic lineage cells can be administered to a subject without being expanded ex vivo or in vitro prior to administration. In particular embodiments, an isolated population of derived hematopoietic lineage cells is modulated and treated ex vivo using one or more agents to obtain immune cells with improved therapeutic potential. In some embodiments, the modulated population of derived hematopoietic lineage cells can be washed to remove the treatment agent(s), and the improved population can be administered to a subject without further expansion of the population in vitro. In some embodiments, an isolated population of derived hematopoietic lineage cells is expanded prior to modulating the isolated population with one or more agents.

In some embodiments, an isolated population of derived hematopoietic lineage cells can be genetically modified according to the methods of the present disclosure to express a recombinant TCR, CAR or other gene product of interest. For genetically engineered derived hematopoietic lineage cells that express a recombinant TCR or CAR, whether prior to or after genetic modification of the cells, the cells can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Cancers

Any cancer can be treated using a cell or pharmaceutical composition described herein. Exemplary therapeutic targets of the present disclosure include cancer cells from the bladder, blood, bone, bone marrow, brain, breast, colon, esophagus, eye, gastrointestinal system, gum, head, kidney, liver, lung, nasopharynx, neck, ovary, prostate, skin, stomach, testis, tongue, or uterus. In addition, a cancer may specifically be of the following non-limiting histological type: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is a breast cancer. In some embodiments, the cancer is colon cancer. In some embodiments, the cancer is gastric cancer. In some embodiments, the cancer is RCC. In another embodiment, the cancer is non-small cell lung cancer (NSCLC).

In some embodiments, solid cancer indications that can be treated with cells described herein (e.g., cells modified using methods of the disclosure, e.g., genetically modified iNK cells), either alone or in combination with one or more additional cancer treatment modality, include: bladder cancer, hepatocellular carcinoma, prostate cancer, ovarian/uterine cancer, pancreatic cancer, mesothelioma, melanoma, glioblastoma, HPV-associated and/or HPV-positive cancers such as cervical and HPV+ head and neck cancer, oral cavity cancer, cancer of the pharynx, thyroid cancer, gallbladder cancer, and soft tissue sarcomas. In some embodiments, hematological cancer indications that can be treated with cells described herein (e.g., cells modified using methods of the disclosure, e.g., genetically modified iNK cells), either alone or in combination with one or more additional cancer treatment modalities, include: ALL, CLL, NHL, DLBCL, AML, CML, and multiple myeloma (MM).

In some embodiments, examples of cellular proliferative and/or differentiative disorders of the lung that can be treated with cells described herein (e.g., cells modified using methods of the disclosure) include, but are not limited to, tumors such as bronchogenic carcinoma, including paraneoplastic syndromes, bronchioloalveolar carcinoma, neuroendocrine tumors, such as bronchial carcinoid, miscellaneous tumors, metastatic tumors, and pleural tumors, including solitary fibrous tumors (pleural fibroma) and malignant mesothelioma.

In some embodiments, examples of cellular proliferative and/or differentiative disorders of the breast that can be treated with cells described herein (e.g., cells modified using methods of the disclosure) include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

In some embodiments, examples of cellular proliferative and/or differentiative disorders involving the colon that can be treated with cells described herein (e.g., cells modified using methods of the disclosure) include, but are not limited to, tumors of the colon, such as non-neoplastic polyps, adenomas, familial syndromes, colorectal carcinogenesis, colorectal carcinoma, and carcinoid tumors.

In some embodiments, examples of cancers or neoplastic conditions, in addition to the ones described above that can be treated with cells described herein (e.g., cells modified using methods of the disclosure), include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

In some embodiments, cells described herein (e.g., cells modified using methods of the disclosure) are used in combination with one or more cancer treatment modalities. In some embodiments, other cancer treatment modalities include, but are not limited to: chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfanide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1 (see, e.g., Agnew, Chem. Intl. Ed. Engl., 1994; 33:183-186); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANET™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; cyclosporine, sirolimus, rapamycin, rapalogs, ibandronate; topoisomerase inhibitor RFS 2000; difluoromethylornithine (DMFO); retinoids such as retinoic acid; CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU, leucovovin; anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); aptamers, described for example in U.S. Pat. No. 6,344,321, which is herein incorporated by reference in its entirety; anti HGF monoclonal antibodies (e.g., AV299 from Aveo, AMG102, from Amgen); truncated mTOR variants (e.g., CGEN241 from Compugen); protein kinase inhibitors that block mTOR induced pathways (e.g., ARQ197 from Arqule, XL880 from Exelexis, SGX523 from SGX Pharmaceuticals, MP470 from Supergen, PF2341066 from Pfizer); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

In some embodiments, cells described herein (e.g., cells modified using methods of the disclosure) are used in combination with one or more cancer treatment modalities that facilitate the induction of antibody dependent cellular cytotoxicity (ADCC) (see e.g., Janeway's Immunobiology by K. Murphy and C. weaver). In some embodiments, such a cancer treatment modality is an antibody. In some embodiments, such an antibody is Trastuzumab. In some embodiments, such an antibody is Rituximab. In some embodiments, such an antibody is Rituximab, Palivizumab, Infliximab, Trastuzumab, Alemtuzumab, Adalimumab, Ibritumomab tiuxetan, Omalizumab, Cetuximab, Bevacizumab, Natalizumab, Panitumumab, Ranibizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Obinutuzumab, Siltuximab, Ramucirumab, Vedolizumab, Blinatumomab, Nivolumab, Pembrolizumab, Idarucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Inotuzumab ozogamicin, Brodalumab, Guselkumab, Dupilumab, Sarilumab, Avelumab, Ocrelizumab, Emicizumab, Benralizumab, Gemtuzumab ozogamicin, Durvalumab, Burosumab, Lanadelumab, Mogamulizumab, Erenumab, Galcanezumab, Tildrakizumab, Cemiplimab, Emapalumab, Fremanezumab, Ibalizumab, Moxetumomab pasudodox, Ravulizumab, Romosozumab, Risankizumab, Polatuzumab vedotin, Brolucizumab, or any combination thereof (see e.g., Lu et al., Development of therapeutic antibodies for the treatment of diseases. Journal of Biomedical Science, 2020). In some embodiments, cells described herein (e.g., cells modified using methods of the disclosure) are used in combination with one or more cancer treatment modalities that facilitate the induction of antibody dependent cellular cytotoxicity (ADCC), wherein the cancer treatment modality is an antibody or appropriate fragment thereof targeting CD20, TNFα, HER2, CD52, IgE, EGFR, VEGF-A, ITGA4, CTLA-4, CD30, VEGFR2, α4β7 integrin, CD19, CD3, PD-1, GD2, CD38, SLAMF7, PDGFRα, PD-L1, CD22, CD33, IFNγ, CD79β, or any combination thereof.

In some embodiments, cells described herein are utilized in combination with checkpoint inhibitors. Examples of suitable combination therapy checkpoint inhibitors include, but are not limited to, antagonists of PD-1 (Pdcdl, CD279), PDL-1 (CD274), TIM-3 (Havcr2), TIGIT (WUCAM and Vstm3), LAG-3 (Lag3, CD223), CTLA-4 (Ctla4, CD152), 2B4 (CD244), 4-1BB (CD137), 4-1BBL (CD137L), A2aR, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, inhibitory KIR (for example, 2DL1, 2DL2, 2DL3, 3DL1, and 3DL2), or any suitable combination thereof.

In some embodiments, the antagonist inhibiting any of the above checkpoint molecules is an antibody. In some embodiments, the checkpoint inhibitory antibodies may be murine antibodies, human antibodies, humanized antibodies, a camel Ig, a shark heavychain-only antibody (VNAR), Ig NAR, chimeric antibodies, recombinant antibodies, or antibody fragments thereof. Non-limiting examples of antibody fragments include Fab, Fab′, F(ab)′2, F(ab)′3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab (anti-PDL1 mAb), avelumab (anti-PDL1 mAb), durvalumab (anti-PDL1 mAb), tremelimumab (anti-CTLA4 mAb), ipilimumab (anti-CTLA4 mAb), IPH4102 (anti-KIR), IPH43 (anti-MICA), IPH33 (anti-TLR3), lirilumab (anti-KIR), monalizumab (anti-NKG2A), nivolumab (anti-PD1 mAb), pembrolizumab (anti-PD 1 mAb), and any derivatives, functional equivalents, or biosimilars thereof.

In some embodiments, the antagonist inhibiting any of the above checkpoint molecules is microRNA-based, as many miRNAs are found as regulators that control the expression of immune checkpoints (Dragomir et al, Cancer Biol Med. 2018, 15(2): 103-115). In some embodiments, the checkpoint antagonistic miRNAs include, but are not limited to, miR-28, miR-15/16, miR-138, miR-342, miR-20b, miR-21, miR-130b, miR-34a, miR-197, miR-200c, miR-200, miR-17-5p, miR-570, miR-424, miR-155, miR-574-3p, miR-513, miR-29c, and/or any suitable combination thereof.

In some embodiments, cells described herein (e.g., cells modified using methods of the disclosure) are used in combination with one or more cancer treatment modalities such as exogenous interleukin (IL) dosing. In some embodiments, an exogenous IL provided to a patient is IL-15. In some embodiments, systemic IL-15 dosing when used in combination with cells described herein is reduced when compared to standard dosing concentrations (see e.g., Waldmann et al., IL-15 in the Combination Immunotherapy of Cancer. Front. Immunology, 2020).

Other compounds that are effective in treating cancer are known in the art and described herein that are suitable for use with the compositions and methods of the present disclosure as additional cancer treatment modalities are described, for example, in the “Physicians' Desk Reference, 62nd edition. Oradell, N.J.: Medical Economics Co., 2008”, Goodman & Gilman's “The Pharmacological Basis of Therapeutics, Eleventh Edition. McGraw-Hill, 2005”, “Remington: The Science and Practice of Pharmacy, 20th Edition. Baltimore, Md.: Lippincott Williams & Wilkins, 2000,” and “The Merck Index, Fourteenth Edition. Whitehouse Station, N.J.: Merck Research Laboratories, 2006”, incorporated herein by reference in relevant parts.

All publications, patents and patent applications cited herein, whether supra or infra, are hereby incorporated by reference in their entirety.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of is meant including, and limited to, whatever follows the phrase “consisting of:” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially” of indicates that the listed elements are required or mandatory, but that no other elements are optional and may or may not be present depending upon whether or not they affect the activity or action of the listed elements.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. The contents of database entries, e.g., NCBI nucleotide or protein database entries provided herein, are incorporated herein in their entirety. Where database entries are subject to change over time, the contents as of the filing date of the present application are incorporated herein by reference. Aspects of the embodiments can be modified, if necessary to employ concepts of the various patents, applications and publications to provide yet further embodiments.

The disclosure is further illustrated by the following examples. The examples are provided for illustrative purposes only. They are not to be construed as limiting the scope or content of the disclosure in any way.

### EXAMPLES

### Example 1: Screening of Guide RNAs for GAPDH

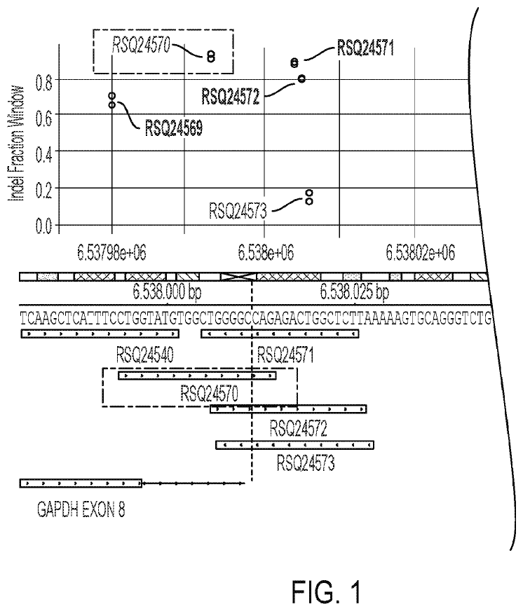

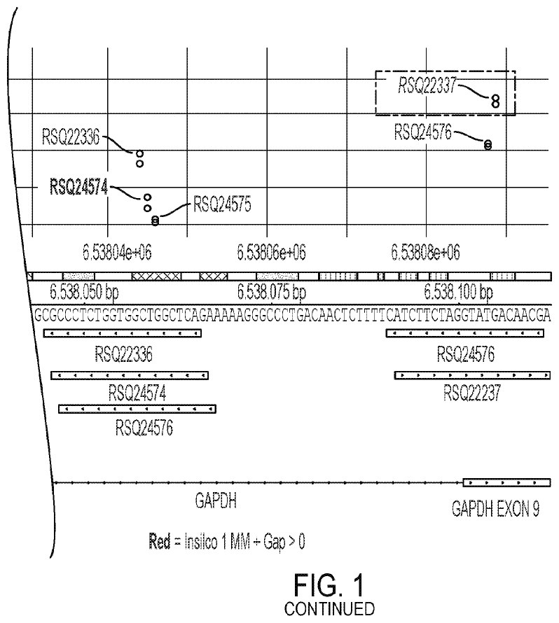

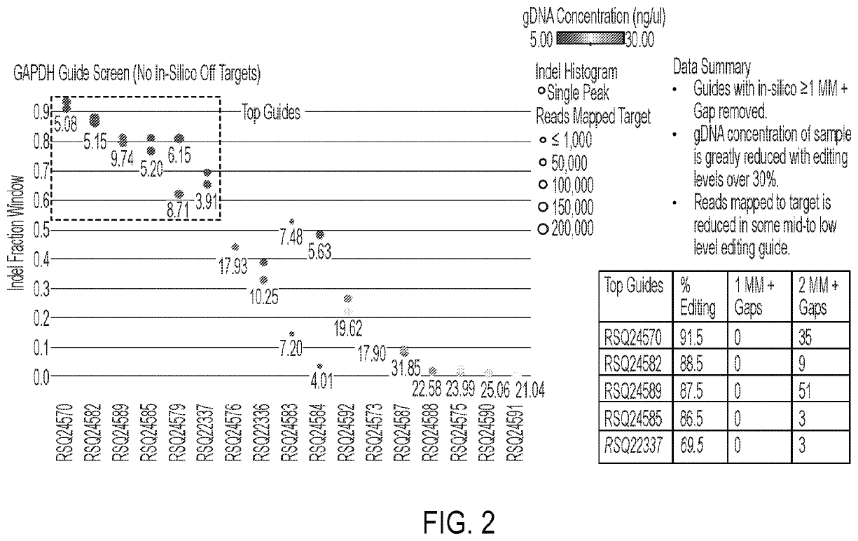

This example describes the screening of AsCpf1 (AsCas12a) guide RNAs that target the housekeeping gene GAPDH. GAPDH encodes Glyceraldehyde-3-Phosphate Dehydrogenase, an essential protein that catalyzes oxidative phosphorylation of glyceraldehyde-3-phosphate in the presence of inorganic phosphate and nicotinamide adenine dinucleotide (NAD), an important energy-yielding step in carbohydrate metabolism. The guide RNAs used in this analysis were all 41-mer RNA molecules with the following design: 5′-UAAUUUCUACUCUUGUAGAU-[21-mer targeting domain sequence]-3′ (SEQ ID NO: 90). For example, the guide RNA denoted RSQ22337 had the following sequence: 5′-UAAUUUCUACUCUUGUAGAUAUCUUCUAGGUAUGACAACGA-3′ (SEQ ID NO: 93) where the 21-mer targeting domain sequence is underlined. The guide RNAs with the targeting domain sequences shown in Table 7 were tested to determine how effective they were at editing GAPDH. Cas12a RNPs (RNPs having an engineered Cas12a (SEQ ID NO: 62)), containing each of these guide RNAs were transfected into iPSCs, and then editing levels were assayed three days after transfection (see e.g., Wong, K. G. et al. CryoPause: A New Method to Immediately Initiate Experiments after Cryopreservation of Pluripotent Stem Cells. Stem Cell Reports 9, 355-365 (2017)). The results are shown in FIG. 1 and FIG. 2. RSQ24570, RSQ24582, RSQ24589, RSQ24585, and RSQ22337 exhibited the greatest levels of measurable editing out of the GAPDH guides tested, editing approximately 70% or more of cells (about 92%, 89%, 88%, 87%, and 70%, respectively). It was observed that cells transfected with gRNAs targeting certain exonic regions yielded much lower amounts of isolatable genomic DNA (gDNA) for analyzing editing efficiency (at day 3 after transfection) when compared to cells transfected with gRNAs targeting intronic regions, indicating that that RNPs with certain exon-targeting gRNAs were cytotoxic to the cells. This suggested that cells edited with gRNAs targeting exonic regions could result in significant cell death due to the introduction of indels within GAPDH leading to expression of a non-functional GAPDH protein or a protein with insufficient function. It was postulated that it might be possible to use a rescue plasmid to repair the gRNA-mediated cleavage site in GAPDH while also knocking in a gene cargo of interest in frame with the repaired GAPDH via HDR, thereby rescuing those cells in which GAPDH is repaired and the cargo of interest is successfully integrated (as shown in FIG. 1 and FIG. 2). Those transfected cells that are edited (the majority of transfected cells, if a highly effective RNA-guided nucleases is used) but do not undergo HDR repair of GAPDH and do not integrate the cargo of interest die over time because they do not have a functioning GAPDH gene. Those cells carrying the cargo of interest would have an advantage due to a fully functioning GAPDH gene as the cells grow and divide, and these cells would be selected for over time. The expected end result would be a population of cells with a very high rate of cargo knock-in within the GAPDH locus.

The data in FIG. 2 suggested that while Cas12a RNP comprising RSQ22337 resulted in an editing level of approximately 70% at 3 days post-transfection, it caused slightly higher levels of toxicity than other exonic guides (RSQ24570, RSQ24582, RSQ24589, and RSQ24585) (see FIG. 2, only about 3.9 ng/μL of gDNA was isolated from edited cells). Thus, the actual editing efficiency was very likely significantly higher than 70%, as many cells had already died by 3 days post-transfection due to the lack of available rescue constructs and NHEJ forming toxic indels. As a result, RSQ22337 was chosen for further testing.

TABLE 7
Guide RNA sequences

| SEQ ID | | gRNA targeting | |
|---|---|---|---|
| NO: | Name | domain sequence (RNA) | Location |
| 94 | RSQ22336 | UGAGCCAGCCACCAGAGGGCG | Intron 8 |
| 95 | RSQ22337 | AUCUUCUAGGUAUGACAACGA | Intron 8/Exon 9 (cut site |
| | | | in exon 9) |
| 96 | RSQ22338 | GCUACAGCAACAGGGUGGUGG | Exon 9 |
| 97 | RSQ24559 | CCAUAAUUUCCUUUCAAGGUG | Intron 7 |
| 98 | RSQ24560 | CUUUCAAGGUGGGGAGGGAGG | Intron 7 |
| 99 | RSQ24561 | AAGGUGGGGAGGGAGGUAGAG | Intron 7 |
| 100 | RSQ24562 | GCAGACCACAGUCCAUGCCAU | Exon 8 |
| 101 | RSQ24563 | CAGACCACAGUCCAUGCCAUC | Exon 8 |
| 102 | RSQ24564 | CCGGAGGGGCCAUCCACAGUC | Exon 8 |
| 103 | RSQ24565 | UAGACGGCAGGUCAGGUCCAC | Exon 8 |
| 104 | RSQ24566 | CUAGACGGCAGGUCAGGUCCA | Exon 8 |
| 105 | RSQ24567 | UCUAGACGGCAGGUCAGGUCC | Exon 8 |
| 106 | RSQ24568 | GCAGGUUUUUCUAGACGGCAG | Exon 8 |
| 107 | RSQ24569 | UCAAGCUCAUUUCCUGGUAUG | Exon 8 |
| 108 | RSQ24570 | CUGGUAUGUGGCUGGGGCCAG | Exon 8/Intron 8 (cut site |
| | | | in intron 8) |
| 109 | RSQ24571 | AGAGCCAGUCUCUGGCCCCAG | Intron 8 |
| 110 | RSQ24572 | AAGAGCCAGUCUCUGGCCCCA | Intron 8 |
| 111 | RSQ24573 | UAAGAGCCAGUCUCUGGCCCC | Intron 8 |
| 112 | RSQ24574 | CUGAGCCAGCCACCAGAGGGC | Intron 8 |
| 113 | RSQ24575 | UCUGAGCCAGCCACCAGAGGG | Intron 8 |
| 114 | RSQ24576 | CAUCUUCUAGGUAUGACAACG | Exon 9 |
| 115 | RSQ24578 | UUGAUGGUACAUGACAAGGUG | 1 kb_downstream |
| 116 | RSQ24579 | GAGGCCCUACCCUCAGUCUGA | 1 kb_downstream |
| 117 | RSQ24580 | CCUCUCCUCGCUCCAGUCCUA | 1 kb_downstream |
| 118 | RSQ24581 | CUCUCCUCGCUCCAGUCCUAG | 1 kb_downstream |
| 119 | RSQ24582 | GCCAACAGCAGAUAGCCUAGG | 1 kb_downstream |
| 120 | RSQ24583 | UGUGCCCUCGUGUCUUAUCUG | 1 kb_downstream |
| 121 | RSQ24584 | CCUAGAUGAAUCCUGCUUGAA | 1 kb_downstream |
| 122 | RSQ24585 | GGUACUUGGUUUACCUAGAUG | 1 kb_downstream |
| 123 | RSQ24586 | AGGUACUUGGUUUACCUAGAU | 1 kb_downstream |
| 124 | RSQ24587 | AAACAUUAUAUAGUCCUUACC | 1 kb_downstream |
| 125 | RSQ24588 | UAAACAUUAUAUAGUCCUUAC | 1 kb_downstream |
| 126 | RSQ24589 | GCGAUUUUUAAACAUUAUAUA | 1 kb_downstream |
| 127 | RSQ24590 | ACCGAUUUUUAAACAUUAUAU | 1 kb_downstream |
| 128 | RSQ24591 | UACCGAUUUUUAAACAUUAUA | 1 kb_downstream |
| 129 | RSQ24592 | AAAAUCGGUAAAAAUGCCCAC | 1 kb_downstream |
| 130 | RSQ24593 | GAGGAAGAUGAACUGAGAUGU | 1 kb_downstream |
| 131 | RSQ24594 | AGGAAGAUGAACUGAGAUGUG | 1 kb_downstream |

### Example 2: Rescue of GAPDH Knock-Out Through Targeted Integration

To test the feasibility of the exemplary selection system illustrated in FIGS. 3A, 3B, and 3C, the essential gene GAPDH was targeted in iPSCs using an RNP comprising AsCpf1 (SEQ ID NO: 62), and a guide RNA (RSQ22337) (SEQ ID NO: 95), resulting in a double-strand break towards the 5′ end of the last exon of GAPDH (exon 9). While iPSCs were tested for the purposes of this experiment, the described methods could be applied to other cell types. RSQ22337 was determined to be highly specific to GAPDH and have minimal off-target sites in the genome (data not shown). GAPDH was thus considered a good exemplary candidate target gene for the cargo integration and selection methods described herein, at least in part because there was at least one highly specific gRNA targeting a terminal exon capable of mediating highly efficient RNA-guided cleavage.

The CRISPR/Cas nuclease and guide RNA were introduced into cells by nucleofection (electroporation) of a ribonucleoprotein (RNP) according to known methods. The cells were also contacted with a double stranded DNA donor template (e.g., a dsDNA plasmid) that included a knock-in cassette comprising in 5′-to-3′ order, a 5′ homology arm approximately 500 bp in length (comprising a portion of exon 8, intron 8, and a 5′ codon-optimized coding portion of exon 9 optimized to prevent further binding of the gRNA targeting domain sequence of the guide RNA (RSQ22337)), an in-frame sequence encoding the P2A self-cleaving peptide (“P2A”), an in-frame coding sequence for CD47 (“Cargo”), a stop codon and polyA signal sequence, and a 3′ homology arm approximately 500 bp in length (comprising a coding portion of exon 9 including a stop codon, the 3′ exonic region of exon 9, and a portion of the downstream intergenic sequence) (as shown in FIG. 3B). The 5′ and 3′ homology arms flanking the knock-in cassette were designed to correspond to sequences surrounding the RNP cleavage site.

As shown schematically in FIG. 3C, NHEJ-mediated creation of indels in cells that are edited by the DNA nuclease but not successfully targeted by the DNA donor template, produce a non-functional version of GAPDH which is lethal to the cells. This knock-out is “rescued” in cells that are successfully targeted by the DNA donor template by correct integration of the knock-in cassette, which restores the GAPDH coding region so that a functioning gene product is produced, and positions the P2A-Cargo sequence in frame with and downstream (3′) of the GAPDH coding sequence. These cells survive and continue to proliferate. Cells that are not edited by the DNA nuclease also continue to proliferate but are expected to represent a very small percentage of the overall cell population, if, as in this case, the editing efficiency of the nuclease in combination with the gRNA is high (see Example 1) and results in creation of a non-functional protein. The editing results for RSQ22337 likely underestimate the actual editing efficiency of the guide due to cell death within the population of edited cells.

The editing efficiency of RNPs containing RSQ22337 were tested at different concentrations (4 μM, 1 μM, 0.25 μM, or 0.0625 μM of RNP) in the absence of double stranded DNA donor template) was first measured at 48 after nucleofection of iPSCs (a time point prior to cell death due to loss of GAPDH gene function). The results show that a concentration of 4 μM resulted in the highest editing levels.

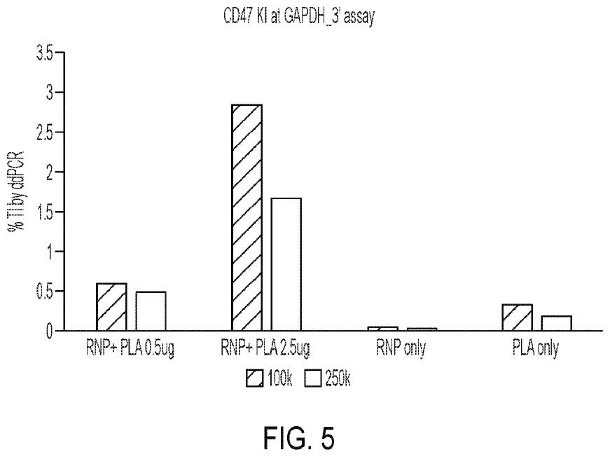

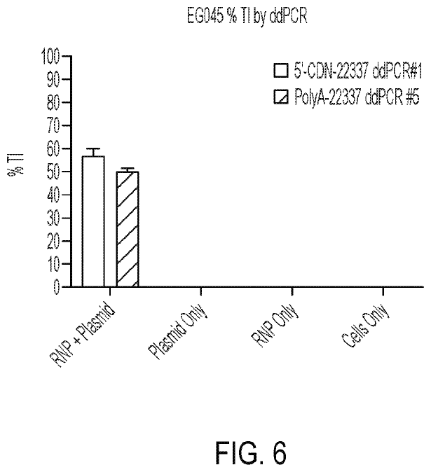

FIGS. 5 and 6 show that a protein-encoding cargo gene can be knocked into a housekeeping gene, such as GAPDH, at high efficiency using the selection systems described herein. FIG. 5 shows the knock-in (KI) efficiency of the CD47-encoding “cargo” in GAPDH at 4 days post-electroporation when RNP was present at a concentration of 4 μM and the dsDNA plasmid (“PLA”) encoding CD47 was also present. Knock-in efficiency was measured with two different concentrations of the plasmid (0.5 μg and 2.5 μg of plasmid) and found to be dose responsive. Knock-in was measured using ddPCR targeting the 3′ position of the knock-in “cargo”. Control cells electroporated with RNP alone or PLA alone exhibited much lower knock-in rates than electroporation of RNP and PLA (at a concentration of 2.5 μg).

FIG. 6 shows the knock-in efficiency of the CD47-encoding “cargo” in GAPDH at 9 days post-electroporation of the cells with the RNP and dsDNA plasmid encoding CD47. The percentage knock-in was similar when either the 5′ end or the 3′ end of the cargo was assayed by ddPCR, using a primer specific for the 5′ of the gRNA target site or 3′ of the site in the poly A region, increasing the reliability of the result. The knock-in efficiency of the cargo was significantly higher at 9 days compared to at 4 days post-transfection (compare FIGS. 5 and 6), consistent with the expectation that there would be substantial cell death in RNP-induced GAPDH knock-out cells that lacked a functional GAPDH gene as a result of unsuccessful cargo knock-in and rescue at GAPDH.

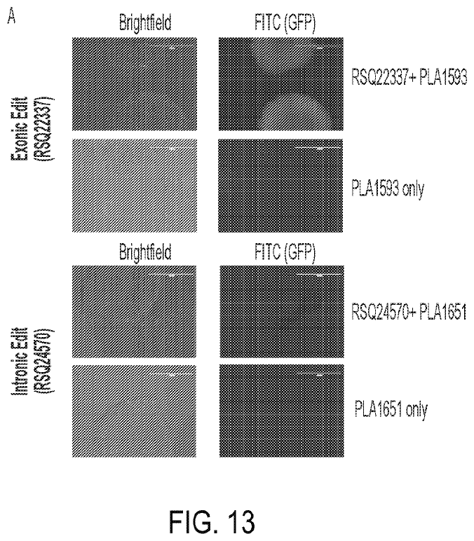

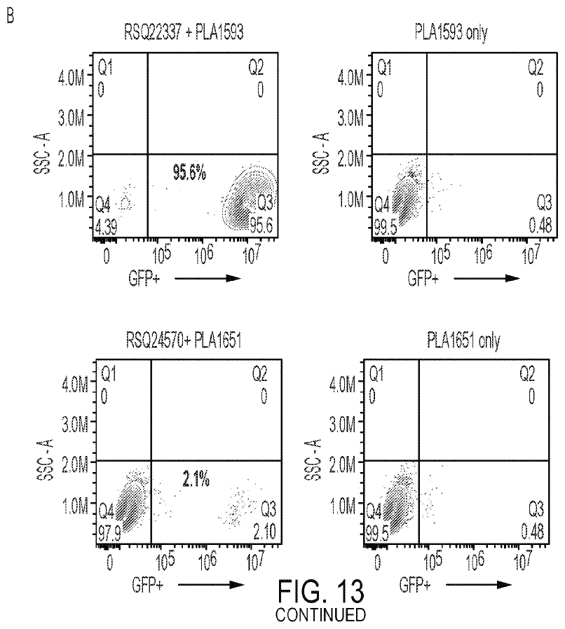

An experiment was then conducted to test the mechanism of the selection system described above by confirming that edited cells containing a successfully knocked-in cargo gene would be more efficiently selected for using a gRNA targeting a protein-coding exonic portion of GAPDH rather than a gRNA targeting an intron. FIG. 13 compares the knock-in efficiency of a GFP-encoding “cargo” knock-in cassette at the GAPDH locus when using a gRNA that mediates cleavage within an intron (RSQ24570 (SEQ ID NO: 108) binds to the exon 8-intron 9 junction, leading to Cas12a-mediated cleavage within intron 8) relative to a gRNA specific for an exon (RSQ22337 (SEQ ID NO: 95), targeting the intron 8-exon 9 junction, leading to Cas12a-mediated cleavage within exon 9). Rescue dsDNA plasmid PLA1593 comprising the reporter “cargo” GFP was nucleofected into iPSCs with an RNP (Cas12a and RSQ22337) targeting GAPDH as described above, while dsDNA plasmid PLA1651 comprising a donor template sequence as depicted in SEQ ID NO: 46 was nucleofected with an RNP comprising Cas12a and RSQ24570. The homology arms of each plasmid were designed to mediate HDR based on the target site of each gRNA. Knock-in was visualized using microscopy (FIG. 13A) and was measured using flow cytometry (FIG. 13B). Knock-in efficiency was significantly higher when using a gRNA and associated knock-in cassette that cleaves at an exonic coding region (exon 9) when compared to an intronic region (intron 8). FIG. 13B shows that 95.6% of cells electroporated with RSQ22337 and the GFP-encoding “cargo” knock-in cassette (e.g., PLA1593; comprising donor template SEQ ID NO: 44) expressed GFP compared to only 2.1% of cells electroporated with RSQ24570 and a GFP-encoding “cargo” knock-in cassette (PLA1651; comprising donor template SEQ ID NO: 46). The results depicted in FIG. 13 are striking, as while the measured editing efficiency (as determined by indel generation frequency 72 hours post-transfection as discussed above in Example 1, see FIG. 2) of RSQ24570 is higher than that of RSQ22337, the proportion of cells rescued by the knock-in construct targeting the coding exonic region are significantly higher.

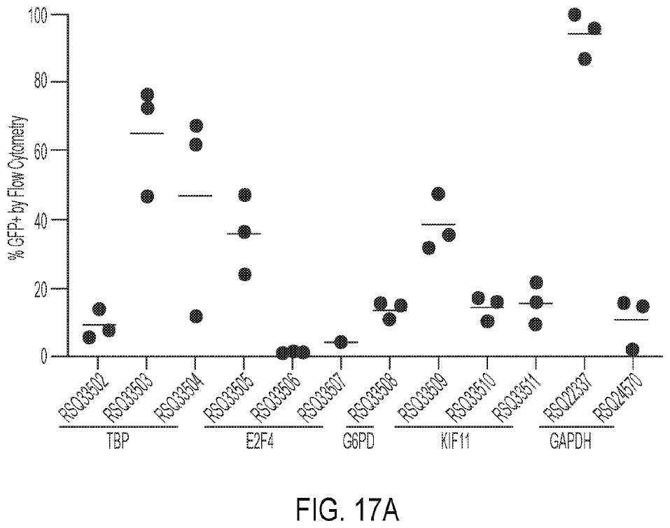

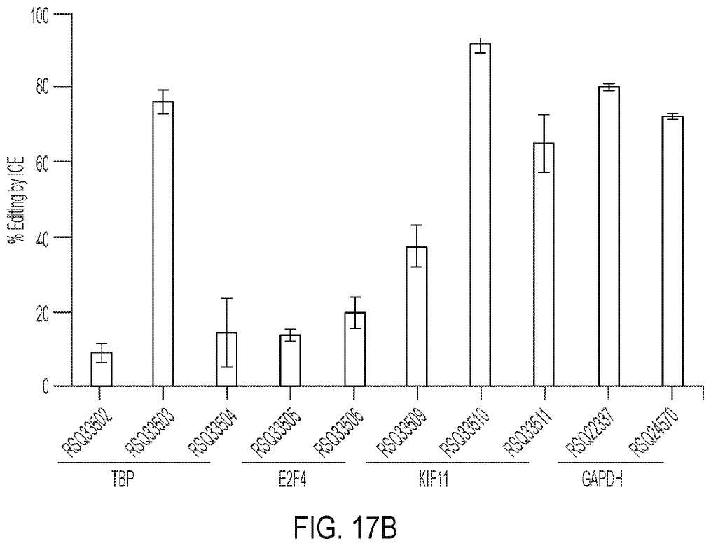

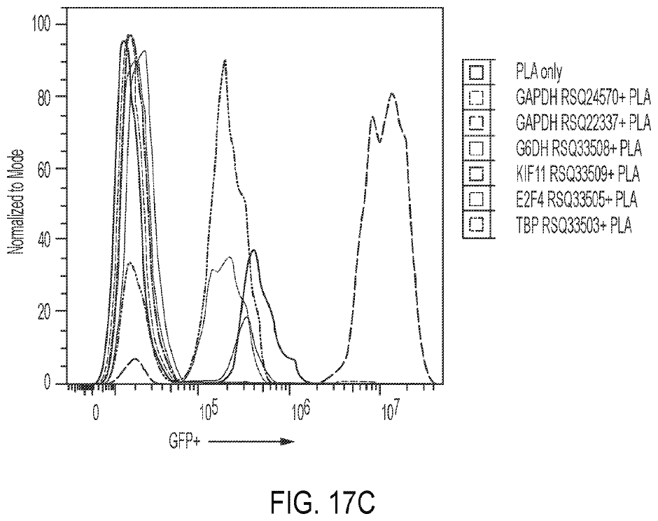

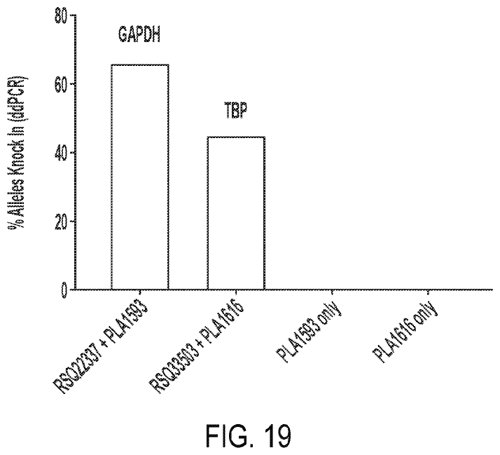

In an additional set of experiments, iPS cells were contacted with an RNP containing AsCas12a (SEQ ID NO: 62), and RSQ22337 (SEQ ID NO: 95) or RSQ24570 (SEQ ID NO: 108), along with either the PLA1593 (comprising donor template SEQ ID NO: 44) or the PLA1651 (comprising donor template SEQ ID NO: 46) double stranded DNA donor template plasmid, respectively, as described above. Flow cytometry was performed 7 days following nucleofection to detect GFP expression and help determine to what extent each plasmid mediated donor template and knock-in cassette was integrated successfully at its respective GAPDH target site. The GAPDH results in FIG. 17A show that cells nucleofected with the RNP containing RSQ22337 exhibited a much higher amount of GFP expression relative to cells nucleofected with RSQ24750, showing that most cells express GFP at day 7 following electroporation. This suggests that the GFP-encoding knock-in cassette integrated successfully at high levels within the RSQ22337-transfected cells. Cells nucleofected with RNPs containing RSQ24750 displayed much lower GFP expression, indicating that the knock-in cassette did not integrate successfully in most of these cells (FIG. 17A). The GAPDH results of FIG. 17B show that use of RSQ22337 resulted in about 80% editing as measured using genomic DNA 48 hours following RNP transfection, while RSQ24570 resulted in about 75% editing as measured using genomic DNA 48 hours following RNP transfection. The high editing of RSQ22337 correlated well with the high GFP expression level depicted in FIG. 17A; however, the high editing of RSQ24750 correlated poorly with the low GFP expression level depicted in FIG. 17A. FIG. 17C shows the relative integrated “cargo” (GFP) expression intensity of the edited cells. Finally, a ddPCR assay was conducted to determine the percentage of knock-in integration events in GAPDH alleles in the cells nucleofected with RNPs containing RSQ22337 and the PLA1593 donor plasmid. FIG. 19 shows by ddPCR that over 60% of alleles had a GFP-encoding cassette knocked-in successfully.

### Example 3: Rescue of GAPDH Knock-Out Through Targeted Integration of Multiple Cargos

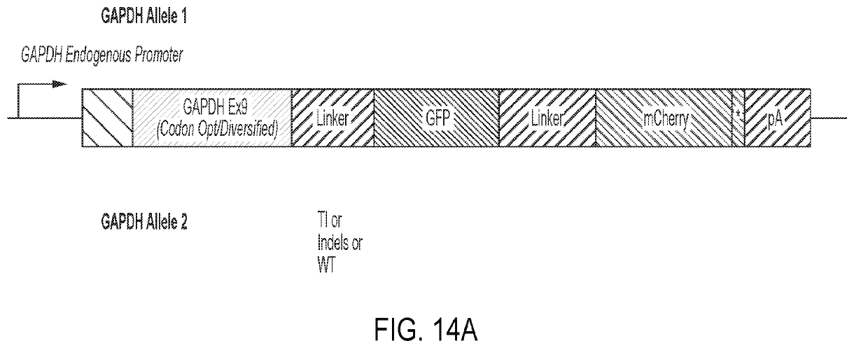

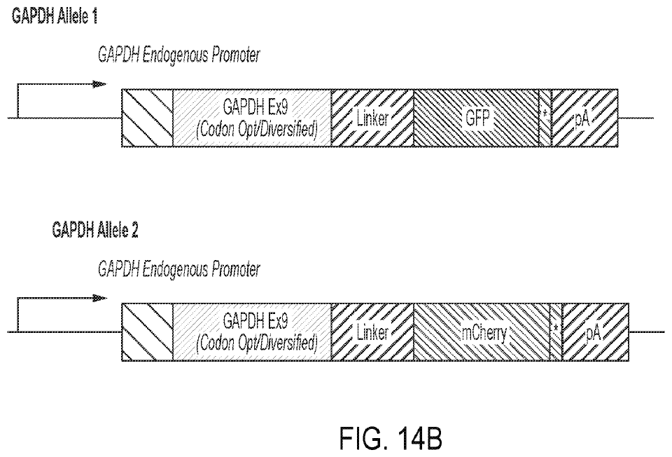

In some cases, it is desirable to use the selection and cargo knock in strategies disclosed herein to efficiently produce and isolate an edited cell containing two or more different exogenous coding sequences, e.g., two or more different exogenous genes, integrated into a single essential gene locus, such as, e.g., the GAPDH locus. FIG. 14 shows two strategies for introducing two or more different exogenous coding regions into an essential gene locus. FIG. 14A shows a first exemplary strategy wherein a multi-cistronic knock-in cassette, e.g., a bi-cistronic knock-in cassette containing two or more coding regions (GFP and mCherry in FIG. 14A), separated by linkers (e.g., T2A, P2A, and/or IRES, see SEQ ID NO: 29-32 and 33-37), is inserted into one or both of the alleles of the essential gene, e.g., GAPDH. FIG. 14B shows a second exemplary strategy (a bi-allelic insertion strategy) wherein two knock-in cassettes comprising different cargo sequences (e.g., different exogenous genes, such as GFP and mCherry in FIG. 14B) are inserted into separate alleles of the essential gene locus, e.g., GAPDH.

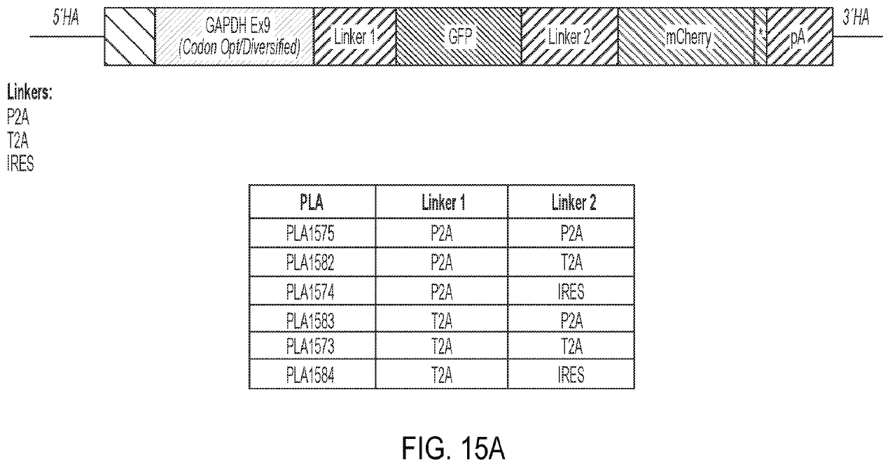

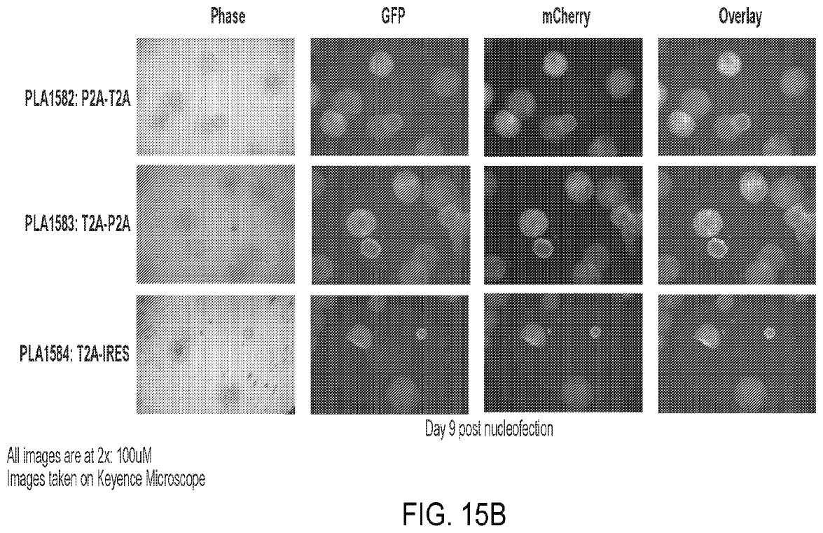

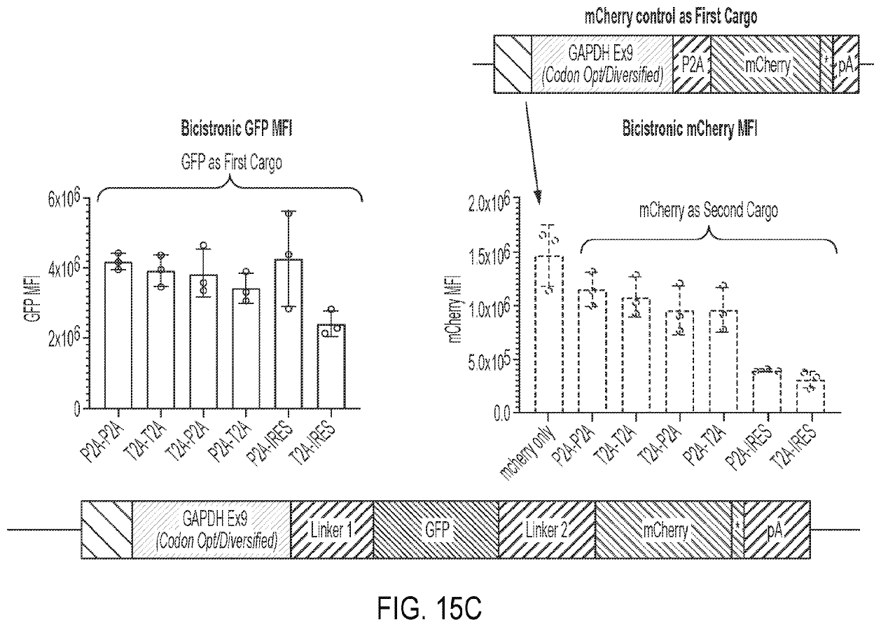

Experiments were conducted to test the integration strategy depicted in FIG. 14A, and to determine whether the use of different combinations of linkers in the knock-in cassette could affect the expression of the cargo sequences. An RNP containing Cas12a and RSQ22337 (targeting the GAPDH locus, as described in Examples 1 and 2) was nucleofected into iPSCs with one of six different plasmids (PLA) containing a bi-cistronic knock-in cassette comprising “cargo” sequences encoding GFP and mCherry (PLA1573, PLA1574, PLA1575, PLA1582, PLA1583, and PLA1584, as depicted in FIG. 15A; comprising donor templates SEQ ID NOs: 38-43). GFP was the first cargo and mCherry was the second cargo in each of these constructs. Each of the tested plasmids contained a different combination of linkers between the coding sequences (Linkers 1 and 2, as depicted in FIG. 15A). PLA1573 (comprising donor template SEQ ID NO: 38) contained T2A and T2A as linkers 1 and 2, respectively; PLA1574 (comprising donor template SEQ ID NO: 39) contained P2A and IRES as linkers 1 and 2, respectively; PLA1575 (comprising donor template SEQ ID NO: 40) contained P2A and P2A as linkers 1 and 2, respectively; PLA1582 (comprising donor template SEQ ID NO: 41) contained P2A and T2A as linkers 1 and 2, respectively; PLA1583 (comprising donor template SEQ ID NO: 42) contained T2A and P2A as linkers 1 and 2, respectively; and PLA1584 (comprising donor template SEQ ID NO: 43) contained T2A and IRES as linkers 1 and 2, respectively. FIG. 15B and FIG. 15C shows the results of various knock-in cassette integration events at the GAPDH locus. FIG. 15B depicts exemplary microscopy images (brightfield and fluorescent microscopy at 2× on a Keyence microscope) of edited iPSCs nine days following nucleofection with exemplary plasmids PLA1582, PLA1583, and PLA1584, each of which exhibited detectable GFP and mCherry expression.

FIG. 15C quantifies the fluorescence levels of GFP and mCherry in the iPSCs nucleofected with the various plasmids described in FIG. 15A containing the bi-cistronic knock-in cassettes with the different described linker pairs (PLA1575, PLA1582, PLA1574, PLA1583, PLA1573, and PLA1584). In each of these bi-cistronic constructs, GFP was always the first cargo and mCherry was always the second cargo. A plasmid containing a knock-in cassette with mCherry as a sole “cargo” (as depicted in FIG. 15C) was also tested as a control. The data show that the expression levels of GFP, as the first cargo, were similar between bicistronic constructs and consistently higher than the expression levels of mCherry, the second cargo. Cells containing the control knock-in cassette containing mCherry as the sole cargo exhibited the highest mCherry expression, suggesting that it is possible to vary (e.g., reduce) expression of a cargo by placing it as the second cargo in a bicistronic cassette. In addition, FIG. 15C shows that placement of an IRES linker immediately prior to the second cargo coding sequence resulted in lower expression of the second cargo when compared to the placement of a P2A or T2A linker prior to the second cargo coding sequence. Thus, the results show that it is possible to differentially modulate (i.e., increase or decrease) the expression of two cargo coding sequences from a multicistronic knock-in cassette by varying the order of the cargos in the cassette (placing a cargo as the first cargo for higher expression, or as the second cargo for lower expression) and by placing particular linkers (P2A or T2A for higher expression; IRES for lower expression) upstream of each of the cargos.

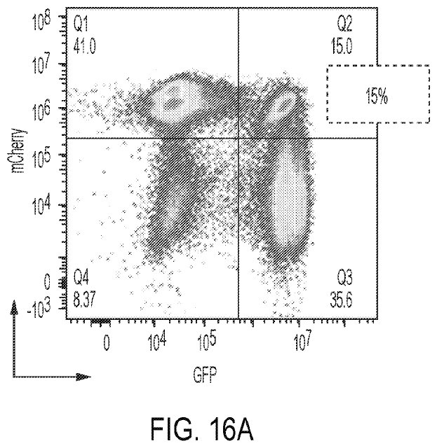

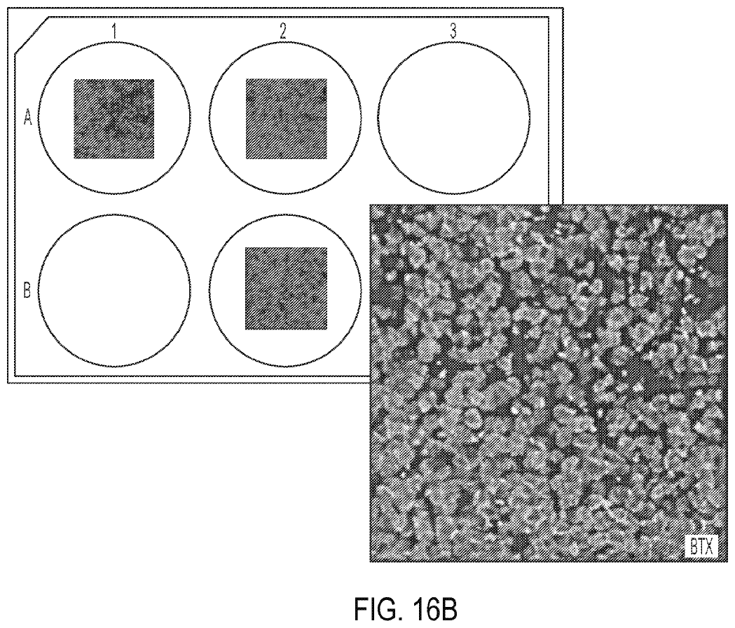

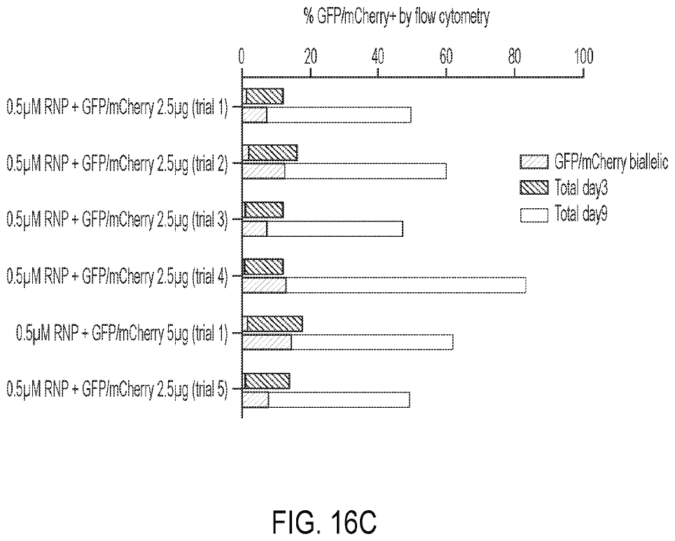

An experiment was conducted to test the bi-allelic integration strategy depicted in FIG. 14B. An RNP containing Cas12a and RSQ22337 (targeting the GAPDH locus, as described in Examples 1 and 2) was nucleofected into iPSCs with two different plasmids. One plasmid contained a knock-in cassette containing a GFP coding sequence as the cargo, and the second plasmid contained a knock-in cassette containing an mCherry coding sequence as the cargo (as depicted in FIG. 14B). FIG. 16A shows exemplary flow cytometry data for the nucleofected iPSCs. Gating showed that a high percentage, approximately 15%, of the nucleofected cells expressed GFP and mCherry, suggesting that the GFP knock-in cassette and the mCherry knock-in cassette were each integrated into an allele of GAPDH. Approximately 41% of the nucleofected cells expressed mCherry and approximately 36% of the nucleofected cells expressed GFP.

An additional experiment was conducted to test biallelic insertion of GFP and mCherry in populations of iPSCs. The iPSC populations were transformed as described. The cells were nucleofected with 0.5 μM RNPs comprising Cas12a and RSQ22337 (targeting the GAPDH locus, as described in Examples 1 and 2), and 2.5 μg of donor template (5 trials) or 5 μg of donor template (1 trial), and then sorted 3 or 9 days following nucleofection. An exemplary image of the edited cell populations that were analyzed by flow cytometry analysis is depicted in FIG. 16B. FIG. 16C provides the flow cytometry analysis results from these trials. The larger bar at each time point (day 3 or day 9) in FIG. 16C represents the total percentage of the cells in each population that positively express at least one cargo, e.g., at least one allele of GFP and/or at least one allele of mCherry cargo. The smaller bar at each time point shows the percentage of cells in each population that express both GFP and mCherry and therefore represents cells with GFP/mCherry biallelic integration. These results showed that approximately 8-15% percent of the transformed cells in each population displayed a biallelic GFP/mCherry insertion phenotype at nine days following transformation.

### Example 4: Rescue of B2M Knock-Out Through Targeted Integration

The approach described in Example 2 is used to target the B2M gene in NK cells (e.g., by targeting NK cells such as iPS-derived NK cells directly or iPS cells that are then differentiated into NK cells). NK cells that lack a functional B2M gene will not be able to recognize MHC Class I on the surface of one another and will attack each other, depleting the population in a phenomenon known as fratricide. By knocking-out the B2M gene and knocking-in a “cargo” sequence that also restores a functional B2M gene one automatically enriches for the knock-in cell type.

### Example 5: Assessment of RPLP0 as a Candidate Essential Gene for Knock-Out Through Targeted Integration

The knock-in integration and selection approach described in Example 2 was evaluated for potential use in targeting other essential genes in cells, e.g., ribosomal genes such as the RPLP0 gene. The RPLP0 gene encodes a ribosomal protein that is a component of the 60S subunit. Ribosomal protein PO is the functional equivalent of E. coli protein L10 and is generally used as a housekeeping gene in RT-qPCR assays.

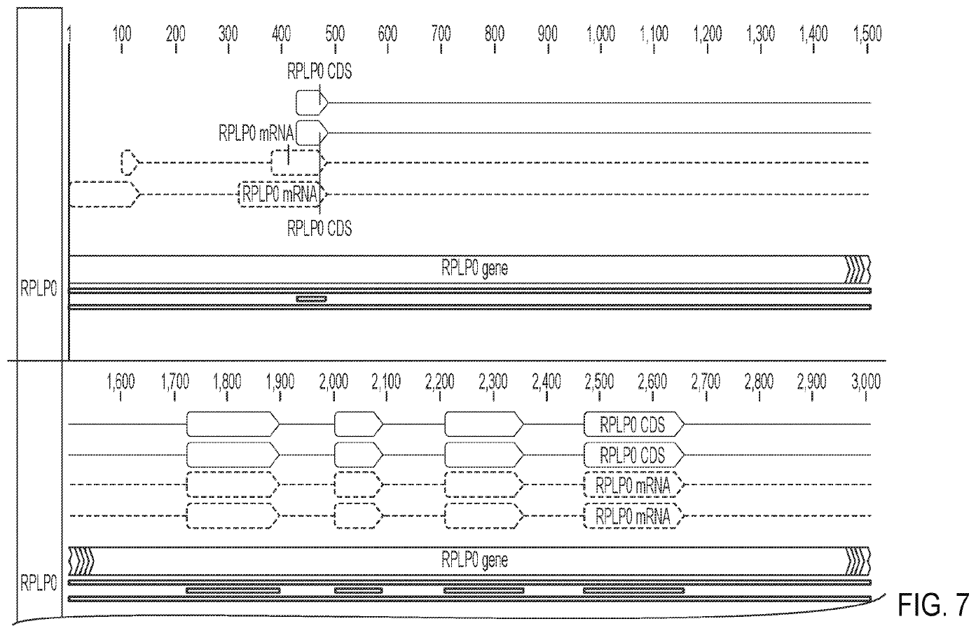

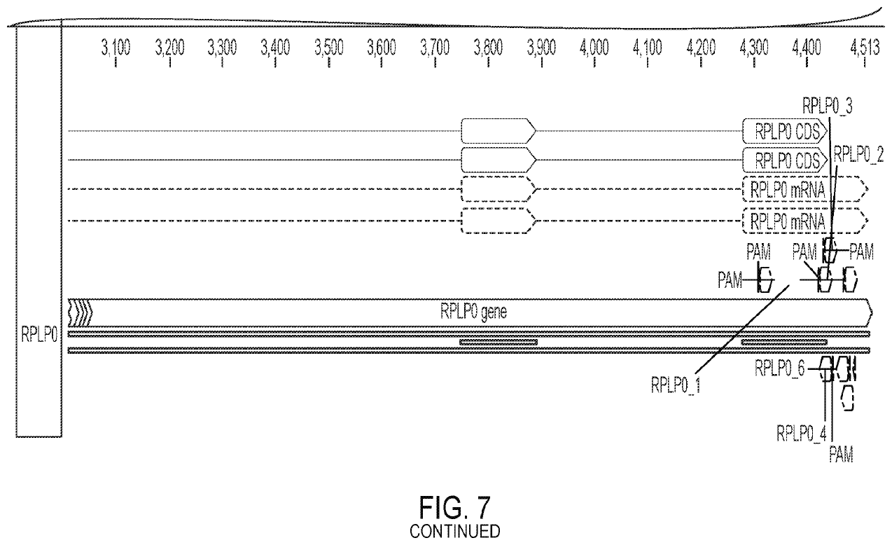

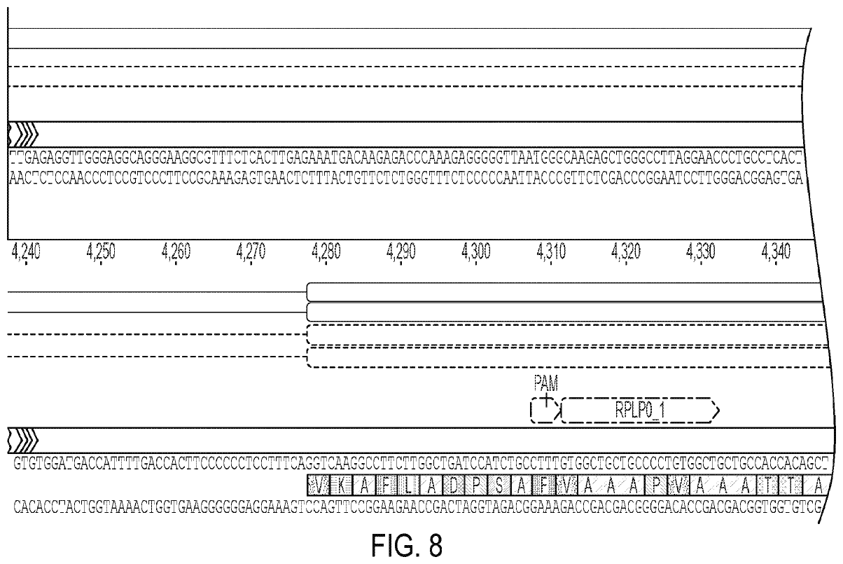

Exemplary AsCpf1 (AsCas12a) guide RNAs that target terminal exons of the RPLP0 gene are shown in Table 8 below. The guide RNAs are all 41-mer RNA molecules with the following design: 5′-UAAUUUCUACUCUUGUAGAU-[21-mer targeting domain sequence]-3′ (SEQ ID NO: 90). FIG. 7 and FIG. 8 map these guides to terminal exons of the RPLP0 gene.

TABLE 8
Guide RNA sequences

| SEQ | | gRNA |
|---|---|---|
| ID NO: | Name | targeting domain sequence (RNA) |
| 132 | RPLP0-1 | UGGCUGCUGCCCCUGUGGCUG |
| 133 | RPLP0-2 | GUCUCUUUGACUAAUCACCAA |
| 134 | RPLP0-3 | ACUAAUCACCAAAAAGCAACC |
| 135 | RPLP0-4 | GUGAUUAGUCAAAGAGACCAA |

However, analysis of potential off-target sites elsewhere in the genome (outside of the RPLP0 locus) for the gRNAs in Table 8 reveal several identical or almost identical target binding sites for the gRNAs in other essential genes associated with ribosomal structure or function, likely due to the highly conserved nature of ribosomal genes (data not shown). Transfecting cells with RNPs containing the gRNAs from Table 8 could potentially kill most of the cells by introducing indels at other essential genes besides RPLP0, even in the presence of a donor plasmid designed to restore the edited RPLP0 gene, as described in Example 2. Additionally, and/or alternatively, off-targets may titrate away RNP complexes from the primary target locus, resulting in a reduced editing rate, and reduction of desired integration events. Thus, these particular gRNA targeting sites in RPLP0 were discounted as possible candidates for a knock-in integration and selection approach as described herein.

### Example 6: Assessment of RPL13A as a Candidate Essential Gene for Knock-Out Through Targeted Integration

The knock-in integration and selection approach described in Example 2 was evaluated for potential use in targeting other essential genes in cells. The RPL13A gene is associated with ribosomes but is not required for canonical ribosome function and has extra-ribosomal functions. It is involved in the methylation of rRNA and is generally used as a housekeeping gene in RT-qPCR assays.

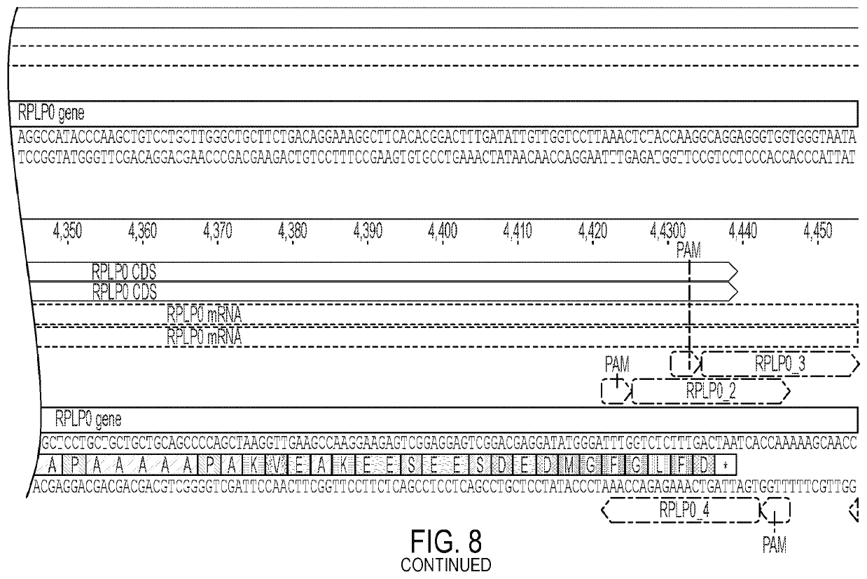

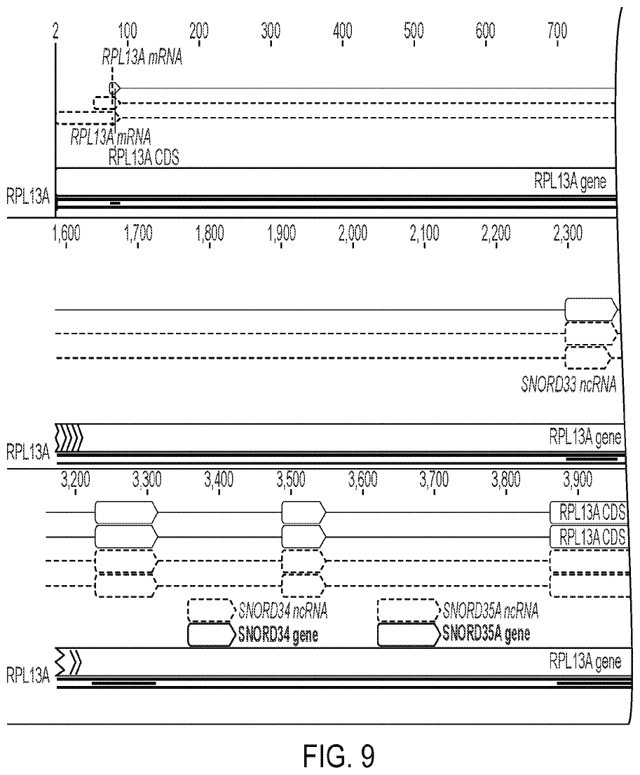

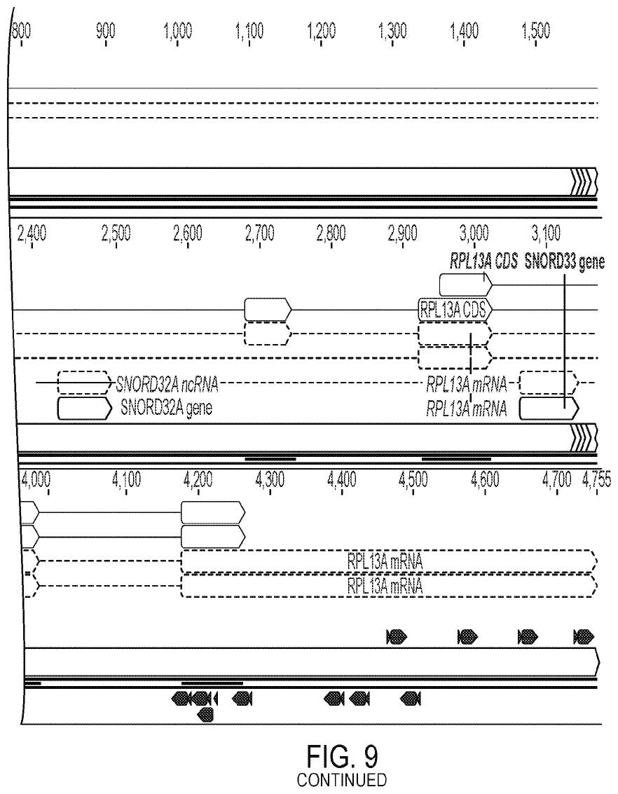

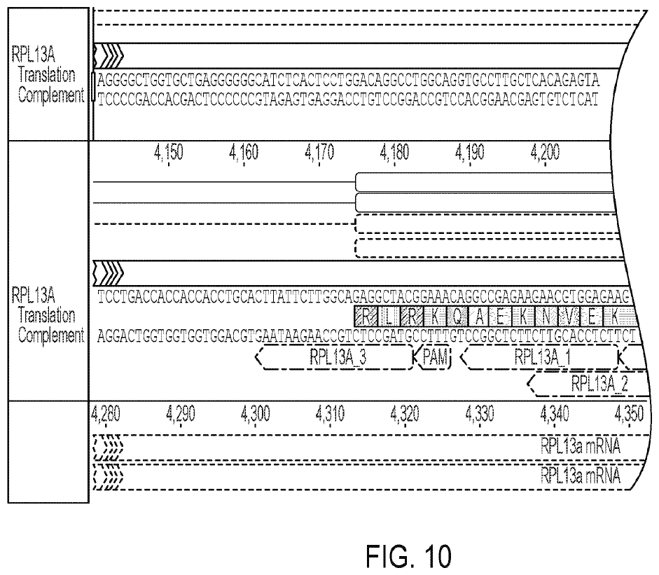

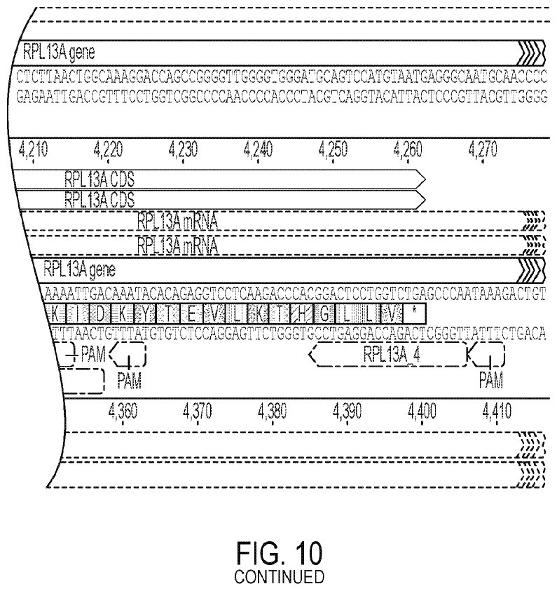

Exemplary AsCpf1 (AsCas12a) guide RNAs that target terminal exons of the RPL13A gene are shown in Table 9 below. The guide RNAs are all 41-mer RNA molecules with the following design: 5′-UAAUUUCUACUCUUGUAGAU-[21-mer targeting domain sequence]-3′ (SEQ ID NO: 90). FIG. 9 and FIG. 10 map these guides to terminal exons of the RPL13A gene.

TABLE 9
Guide RNA sequences

| SEQ | | gRNA |
|---|---|---|
| ID NO: | Name | targeting domain sequence (RNA) |
| 136 | RPL13A-1 | UUCUCCACGUUCUUCUCGGCC |
| 137 | RPL13A-2 | UCAAUUUUCUUCUCCACGUUC |
| 138 | RPL13A-3 | CGUAGCCUCUGCCAAGAAUAA |
| 139 | RPL13A-4 | UUGGGCUCAGACCAGGAGUCC |

However, analysis of potential off-target sites elsewhere in the genome (outside of the RPL13A locus) for the gRNAs in Table 9 reveal several identical or almost identical target binding sites for the gRNAs in other essential genes associated with ribosomal structure or function, likely due to the highly conserved nature of ribosomal genes (data not shown). Transfecting cells with RNPs containing the gRNAs from Table 9 could potentially kill most of the cells by introducing indels at other essential genes besides RPL13A, even in the presence of a donor plasmid designed to restore the edited RPL13A gene, as described in Example 2. Additionally, and/or alternatively, off-targets may titrate away RNP complexes from the primary target locus, resulting in a reduced editing rate, and reduction of desired integration events. Thus, these particular gRNA targeting sites in RPL13A were discounted as possible candidates for a knock-in integration and selection approach as described herein.

### Example 7: Assessment of RPL7 as a Candidate Essential Gene for Knock-Out Through Targeted Integration

The knock-in integration and selection approach described in Example 2 was evaluated for potential use in targeting other essential genes in cells, e.g., ribosomal genes such as the RPL7 gene in cells. The RPL7 gene encodes a ribosomal protein that is a component of the 60S subunit. This ribosomal protein binds to G-rich structures in 28S rRNA and in mRNA and plays a regulatory role in the translation apparatus.

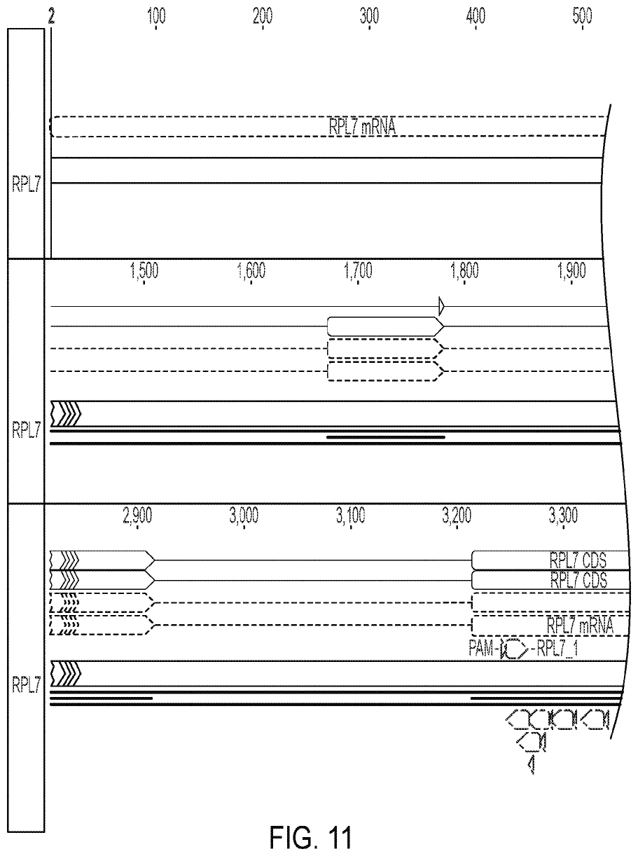

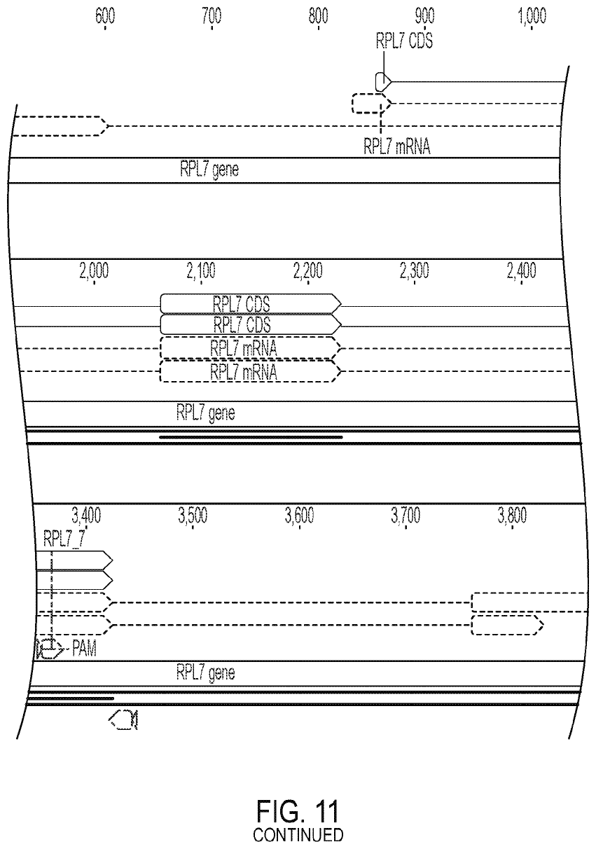

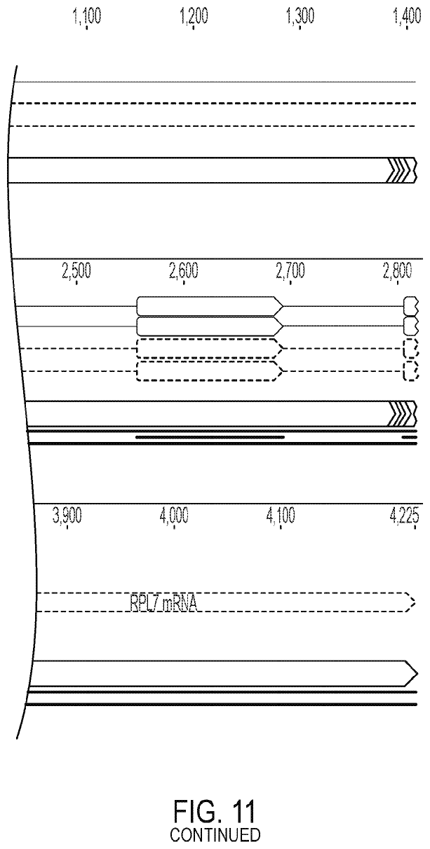

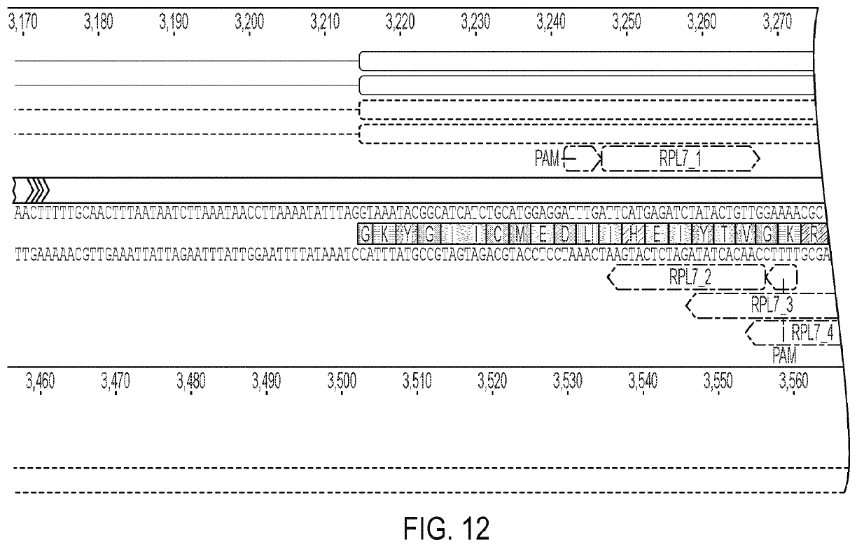

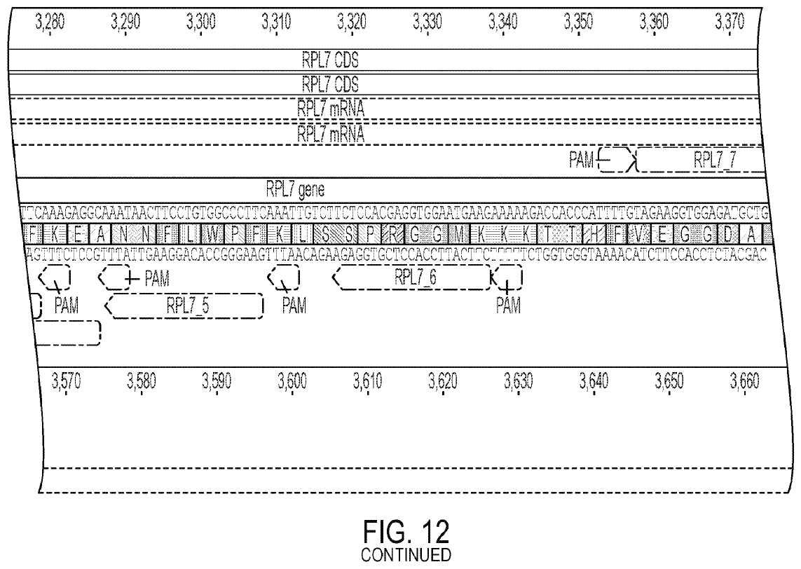

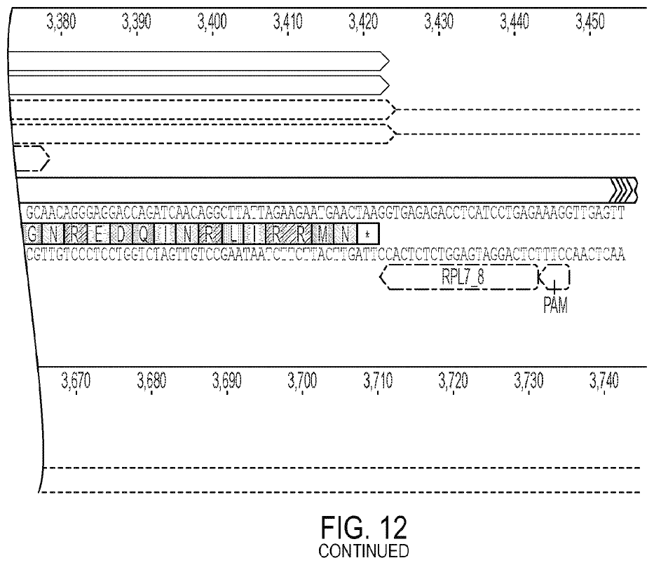

Exemplary AsCpf1 (AsCas12a) guide RNAs that target terminal exons of the RPL7 gene are shown in Table 10 below. The guide RNAs are all 41-mer RNA molecules with the following design: 5′-UAAUUUCUACUCUUGUAGAU-[21-mer targeting domain sequence]-3′ (SEQ ID NO: 90). FIG. 11 and FIG. 12 map these guides to terminal exons of the RPL7 gene.

TABLE 10
Guide RNA sequences

| SEQ | | gRNA |
|---|---|---|
| ID NO: | Name | targeting domain sequence (RNA) |
| 140 | RPL7-1 | AUUCAUGAGAUCUAUACUGUU |
| 141 | RPL7-2 | CAACAGUAUAGAUCUCAUGAA |
| 142 | RPL7-3 | AAGCGUUUUCCAACAGUAUAG |
| 143 | RPL7-4 | CCUCUUUGAAGCGUUUUCCAA |
| 144 | RPL7-5 | AAGGGCCACAGGAAGUUAUUU |
| 145 | RPL7-6 | UUCAUUCCACCUCGUGGAGAA |
| 146 | RPL7-7 | GUAGAAGGUGGAGAUGCUGGC |
| 147 | RPL7-8 | UCAGGAUGAGGUCUCUCACCU |

However, analysis of potential off-target sites elsewhere in the genome (outside of the RPL7 locus) for the gRNAs in Table 10 reveal several identical or almost identical target binding sites for the gRNAs in other essential genes associated with ribosomal structure or function, likely due to the highly conserved nature of ribosomal genes (data not shown). Transfecting cells with RNPs containing the gRNAs from Table 10 could potentially kill most of the cells by introducing indels at other essential genes besides RPL7, even in the presence of a donor plasmid designed to restore the edited RPL7 gene, as described in Example 2. Additionally, and/or alternatively, off-targets may titrate away RNP complexes from the primary target locus, resulting in a reduced editing rate, and reduction of desired integration events. Thus, these particular gRNA targeting sites in RPL7 were discounted as possible candidates for a knock-in integration and selection approach as described herein.

### Example 8: Rescue of TBP Knock-Out Through Targeted Integration

The knock-in integration and selection approach described in Example 2 was used to target the TBP gene in iPSCs. While iPSCs were tested for the purposes of this experiment, the described methods could be applied to other cell types. The TBP gene encodes TATA-box binding protein, a transcriptional regulator that plays a key role in the transcription initiation apparatus. AsCpf1 (AsCas12a) guide RNAs that target terminal exons of the TBP gene are shown in Table 11 below. The guide RNAs are all 41-mer RNA molecules with the following design: 5′-UAAUUUCUACUCUUGUAGAU-[21-mer targeting domain sequence]-3′ (SEQ ID NO: 90).

TABLE 11
Guide RNA sequences

| | Target | gRNA targeting | | |
|---|---|---|---|---|
| Name | Site | domain sequence (RNA) | Location | Plasmid |
| TBP-1 | RSQ33502 | AAAUGCUUCAUAAAUUUCUGC | Isoform 1 exon 8; | PLA1615 |
| | (SEQ ID | | isoform 2 exon 7 | |
| | NO: 148) | | | |
| TBP-2 | RSQ33503 | UGCUCUGACUUUAGCACCUAA | Isoform 1 exon 8; | PLA1616 |
| | (SEQ ID | | isoform 2 exon 7 | |
| | NO: 149) | | | |
| TBP-3 | RSQ33504 | AAAACAUGUACCCUAUUCUAA | Isoform 1 exon 8; | PLA1617 |
| | (SEQ ID | | isoform 2 exon 7 | |
| | NO: 150) | | | |

RSQ33502, RSQ33503, and RSQ33504 (SEQ ID NO: 148-150) described in Table 11 were each determined to be highly specific to TBP and have minimal off-target sites in the genome (data not shown). The TBP gene was thus considered a good candidate gene target for the cargo integration and selection methods described herein at least in part because there are gRNAs available capable of very specifically targeting a terminal exon (mRNA isoform 1 exon 8, or mRNA isoform 2 exon 7 respectively). However, for any of these gRNAs to be highly suitable for the methods described herein, they need to be highly effective at introducing indels at a location in the TBP locus that would knock out and/or severely reduce gene function.

Each of these gRNAs was then tested to determine whether it could be used to knock-in a cassette comprising a portion of TBP and an in-frame cargo sequence encoding GFP into a terminal exon of the TBP gene of cells, in the process rescuing the lethal phenotype that would otherwise result by introducing RNP-induced indels into the coding region of this essential gene. If the tested gRNA was effective at introducing indels at a location of TBP important for function at a high frequency, then transfected cells that do not undergo HDR to incorporate the knock-in cassette would be expected to die, resulting in a large population of the cells expressing GFP from the TBP locus. Specifically, iPSC cells were contacted with an RNP containing AsCas12a (SEQ ID NO: 62), and RSQ33502, RSQ33503 or RSQ33504 (SEQ ID NOs: 148-150), along with a double stranded DNA donor template (dsDNA plasmid) designed to mediate HDR at each respective gRNA target binding site. The double stranded DNA donor templates included a knock-in cassette with a coding sequence for GFP (“Cargo”) in frame with and downstream (3′) of a codon optimized version of a portion of the final TBP exon coding sequence (mRNA isoform 1 exon 8, or mRNA isoform 2 exon 7 respectively) and a sequence encoding the P2A self-cleaving peptide (“P2A”), similar to the dsDNA plasmid described in Example 2 for GAPDH. The TBP sequence in the double stranded DNA donor templates (PLA1615, PLA1616, or PLA1617; comprising donor template SEQ ID NOs: 47, 49, or 50) was codon optimized to prevent further binding by the accompanying guide RNA molecule (RSQ33502, RSQ33503 or RSQ33504). The knock-in cassette also included 3′ UTR and polyA signal sequences downstream of the Cargo sequence. An RNP containing RSQ33502 was administered with PLA1615 (comprising donor template SEQ ID NO: 47); RSQ33503 was administered with PLA1616 (comprising donor template SEQ ID NO: 49); and RSQ33504 was administered with PLA1617 (comprising donor template SEQ ID NO: 50). Each particular dsDNA plasmid (PLA) contained a donor template with homology arms and a knock-in cassette designed to specifically encompass and render ineffective the particular gRNA target site following knock-in cassette integration.

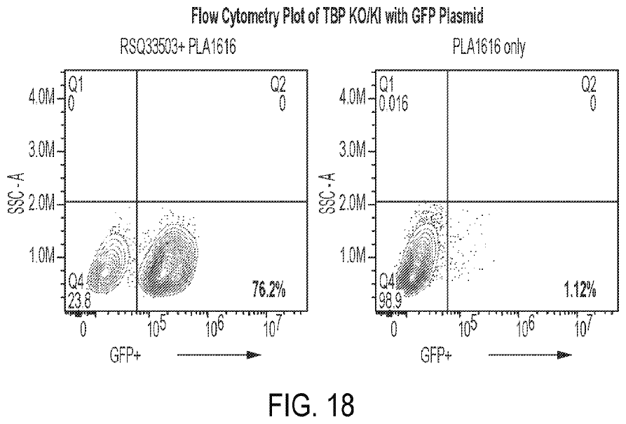

Flow cytometry was performed 7 days following nucleofection and was used to help determine to what extent each plasmid based knock-in cassette was integrated successfully at its respective TBP target site. FIG. 17A shows that cells nucleofected with RNPs containing RSQ33503 exhibited the greatest amounts of GFP expression relative to cells nucleofected with the other RNPs, suggesting that the GFP-encoding knock-in cassette integrated successfully at high levels within these cells. FIG. 18 shows that approximately 76% of the cells nucleofected with RNPs containing RSQ33503 (SEQ ID NO: 149) and the PLA1616 (comprising donor template SEQ ID NO: 49) plasmid expressed GFP compared to only about 1% of cells nucleofected with the PLA1616 plasmid alone (no RNP control). Cells nucleofected with RNPs containing RSQ33504 (SEQ ID NO: 150) also exhibited high levels of GFP expression, also suggesting higher knock-in cassette integration levels (FIG. 17A). Cells nucleofected with RNPs containing RSQ33502 (SEQ ID NO: 148) displayed much lower GFP expression, indicating that the knock-in cassette did not integrate successfully in most of these cells (FIG. 17A). FIG. 17B shows that use of the RNP containing RSQ33503 (SEQ ID NO: 149) resulted in about 80% editing, which correlated with the higher GFP expression level depicted in FIG. 17A. The percentage editing was measured two days following transfection and was determined by ICE assays (as described in Hsiau et al., Inference of CRISPR Edits from Sanger Trace Data. BioRxiv, 251082, August 2019). Use of the RNP containing RSQ33502 (SEQ ID NO: 148) resulted in a relatively low editing percentage, which correlated with the low GFP expression in FIG. 17A. FIG. 17C shows the relative integrated “cargo” (GFP) expression intensity of the edited cells. Finally, a ddPCR assay was conducted to determine the percent knock-in of the GFP cargo into the TBP alleles of the cells nucleofected with RNPs containing RSQ33503 (SEQ ID NO: 149) and the PLA1616 donor plasmid (comprising donor template SEQ ID NO: 49). FIG. 19 shows by ddPCR that over 40% of the TBP alleles had the GFP-encoding cassette successfully knocked-in.

### Example 9: Rescue of E2F4 Knock-Out Through Targeted Integration

The knock-in integration and selection approach described in Example 2 was used to target the E2F4 gene in iPSCs. While iPSCs were tested for the purposes of this experiment, the described methods could be applied to other cell types. The E2F4 gene encodes E2F Transcription Factor 4. This transcriptional regulator plays a key role in cell cycle regulation. AsCpf1 (AsCas12a) guide RNAs that target terminal exons of the E2F4 gene are shown in Table 12 below. The guide RNAs are all 41-mer RNA molecules with the following design: 5′-UAAUUUCUACUCUUGUAGAU-[21-mer targeting domain sequence]-3′ (SEQ ID NO: 90).

TABLE 12
Guide RNA sequences

| | Target | gRNA targeting | | |
|---|---|---|---|---|
| Name | Site | domiain sequence (RNA) | Location | Plasmid |
| E2F4-1 | RSQ33505 | CCCCUCUGCUUCGUCUUUCUC | Exon 10 | PLA1626 |
| | (SEQ ID NO: 151) | | | |
| E2F4-2 | RSQ33506 | UCCACCCCCGGGAGACCACGA | Exon 10 | PLA1627 |
| | (SEQ ID NO: 152) | | | |
| E2F4-3 | RSQ33507 | AUGUGCCUGUUCUCAACCUCU | Exon 10 | PLA1628 |
| | (SEQ ID NO: 153) | | | |

RSQ33505, RSQ33506, and RSQ33507 (SEQ ID NOs: 151-153) were each determined to be highly specific to E2F4 and have minimal off-target sites in the genome (data not shown). The E2F4 gene was thus considered a good candidate gene target for the cargo integration and selection methods described herein at least in part because there are gRNAs available that are capable of very specifically targeting a terminal exon (exon 10). However, for any of these gRNAs to be highly suitable for the methods described herein, they need to be highly effective at introducing indels at a location in the E2F4 locus that would knock out or severely reduce gene function.

The gRNAs RSQ33505, RSQ33506, and RSQ33507 (SEQ ID NOs: 151-153) were then tested to determine whether they could be used to knock-in a cassette comprising a portion of E2F4 and a cargo sequence encoding GFP into a terminal exon of the E2F4 locus of cells, in the process rescuing the lethal phenotype that would otherwise result by introducing RNP-induced indels into the coding region of this essential gene at a high frequency. Specifically, iPSCs were contacted with an RNP containing AsCas12a (SEQ ID NO: 62), and RSQ33505, RSQ33506, or RSQ33507 (SEQ ID NOs: 151-153) along with a double stranded DNA donor template (dsDNA plasmid) designed to mediate HDR at each respective gRNA target binding site. The double stranded DNA donor templates included a knock-in cassette with a coding sequence for GFP (“Cargo”) in frame with and downstream (3′) of a codon optimized version of the final E2F4 exon coding sequence (exon 10) and a sequence encoding the P2A self-cleaving peptide (“P2A”), similar to the dsDNA plasmid described in Example 2 for GAPDH. The E2F4 sequence in the double stranded DNA donor templates (PLA1626, PLA1627, or PLA1628; comprising donor template SEQ ID NOs: 52-54) was codon optimized to prevent further binding by the accompanying guide RNA molecule (RSQ33505, RSQ33506 or RSQ33507; SEQ ID NOs: 151-153). The knock-in cassette also included 3′ UTR and polyA signal sequences downstream of the Cargo sequence. An RNP containing RSQ33505 (SEQ ID NO: 151) was administered with PLA1626 (comprising donor template SEQ ID NO: 52); RSQ33506 (SEQ ID NO: 152) was administered with PLA1627 (comprising donor template SEQ ID NO: 53); and RSQ33507 (SEQ ID NO: 153) was administered with PLA1628 (comprising donor template SEQ ID NO: 54). Each particular dsDNA plasmid (PLA) contained a donor template with homology arms and a knock-in cassette designed to specifically encompass and render ineffective the particular gRNA target site following integration.

Flow cytometry was performed 7 days following nucleofection and was used to help determine to what extent each plasmid based knock-in cassette was integrated successfully at its respective E2F4 target site. FIG. 17A shows that cells nucleofected with RNPs containing RSQ33505 (SEQ ID NO: 151) exhibited the greatest amount of GFP expression relative to cells nucleofected with the other RNPs targeting E2F4, suggesting that the GFP-encoding knock-in cassette integrated successfully in many of these cells. Cells nucleofected with RNPs containing RSQ33506 or RSQ33507 (SEQ ID NOs: 152 and 153) displayed much lower GFP expression, indicating that the knock-in cassette did not integrate successfully in most of these cells (FIG. 17A). FIG. 17B shows that use of RNP containing RSQ33505 (SEQ ID NO: 151) or RSQ33506 (SEQ ID NO: 152) resulted in approximately 15% and approximately 20% editing rates respectively, when measured 48 hours after RNP transfection. The relatively lower observed editing rate for RSQ33505 (SEQ ID NO: 151) may be considered to unexpectedly correlate with a relatively high level of GFP integration in E2F4 (as observed in FIG. 17A), and could partially be the result of significant death within the population of edited cells at 48 hours. The percentage editing was measured two days following transfection and was determined by ICE assays (as described in Hsiau et al., August 2019). FIG. 17C shows the relative integrated “cargo” (GFP) expression intensity of the edited cells.

### Example 10: Rescue of G6PD Knock-Out Through Targeted Integration

The knock-in integration and selection approach described in Example 2 was used to target the G6PD gene in iPSCs. While iPSCs were tested for the purposes of this experiment, the described methods could be applied to other cell types. The G6PD gene encodes Glucose-6-Phosphate Dehydrogenase. This metabolic enzyme plays a key role in glycolysis and NADPH production. An AsCpf1 (AsCas12a) guide RNA that targets terminal exons of the G6PD gene is shown in Table 13 below.

TABLE 13
Guide RNA sequences

| | Target | gRNA targeting | | |
|---|---|---|---|---|
| Name | Site | domain sequence (RNA) | Location | Plasmid |
| G6PD-1 | RSQ33508 | CAGUAUGAGGGCACCUACAAG | Exon 13 | PLA1618 |
| | (SEQ ID NO: 154) | | | |

RSQ33508 (SEQ ID NO: 154) was determined to be highly specific to G6PD and has minimal off-target sites in the genome (data not shown). The G6PD gene was thus considered a good candidate gene target for the cargo integration and selection methods described herein at least in part because there are gRNAs available that are capable of specifically targeting a terminal exon (exon 13).

The gRNA RSQ33508 (SEQ ID NO: 154) was then tested to determine whether it could be used to knock-in a cassette comprising a portion of G6PD and a cargo sequence encoding GFP into a terminal exon of the G6PD locus of cells, in the process rescuing the lethal phenotype that would otherwise result by introducing RNP-induced indels into the coding region of this essential gene at a high frequency. Specifically, iPSCs were contacted with an RNP containing AsCas12a (SEQ ID NO: 62), and RSQ33508 (SEQ ID NO: 154) along with a double stranded DNA donor template (dsDNA plasmid) designed to mediate HDR at the gRNA target binding site. The double stranded DNA donor templates included a knock-in cassette with a coding sequence for GFP (“Cargo”) in frame with and downstream (3′) of a codon optimized version of the final G6PD exon coding sequence (exon 13) and a sequence encoding the P2A self-cleaving peptide (“P2A”), similar to the dsDNA plasmid described in Example 2 for GAPDH. The G6PD sequence in the double stranded DNA donor templates (PLA1618; comprising donor template SEQ ID NO: 51) was codon optimized to prevent further binding by the accompanying guide RNA molecule (RSQ33508). The knock-in cassette also included 3′ UTR and polyA signal sequences downstream of the Cargo sequence. An RNP containing RSQ33508 (SEQ ID NO: 154) was administered with PLA1618 (comprising donor template SEQ ID NO: 51). The dsDNA plasmid (PLA) contained a donor template with homology arms and a knock-in cassette designed to specifically encompass and render ineffective the accompanying gRNA target site following integration.

Flow cytometry was performed 7 days following nucleofection and was used to help determine to what extent the plasmid based knock-in cassette was integrated successfully at its G6PD target site. FIG. 17A shows that cells nucleofected with RNPs containing RSQ33508 (SEQ ID NO: 154) exhibited GFP expression in approximately 10% of assayed cells, suggesting that the GFP-encoding knock-in cassette integrated at relatively low levels within these cells. FIG. 17C shows the relative integrated “cargo” (GFP) expression intensity of the edited cells.

### Example 11: Rescue of KIF11 Knock-Out Through Targeted Integration

The knock-in integration and selection approach described in Example 2 was used to target the KIF11 gene in iPSCs. While iPSCs were tested for the purposes of this experiment, the described methods could be applied to other cell types. The KIF11 gene encodes Kinesin Family Member 11. This enzyme plays a key role in vesicle movement along intracellular microtubules and chromosome positioning during mitosis. AsCpf1 (AsCas12a) guide RNAs that target terminal exons of the KIF11 gene are shown in Table 14 below.

TABLE 14
Guide RNA sequences

| | | gRNA targeting | | |
|---|---|---|---|---|
| Name | Target Site | domain sequence (RNA) | Location | Plasmid |
| KIF11-1 | RSQ33509 | CCGCCUUAAAUCCACAGCAUA | Intron 21/ | PLA1629 |
| | (SEQ ID NO: 155) | | Exon 22 | |
| KIF11-2 | RSQ33510 | UAACCAAGUGCUCUGUAGUUU | Exon 22 | PLA1630 |
| | (SEQ ID NO: 156) | | | |
| KIF11-3 | RSQ33511 | GACCUCUCCAGUGUGUUAAUG | Exon 22 | PLA1631 |
| | (SEQ ID NO: 157) | | | |

RSQ33509, RSQ33510, and RSQ33511 (SEQ ID NOs: 155-157) were each determined to be highly specific to KIF11 and have minimal off-target sites in the genome (data not shown). The KIF11 gene was thus considered a good candidate gene target for the cargo integration and selection methods described herein at least in part because there are gRNAs available that are capable of very specifically targeting a terminal exon available (exon 22). However, for any of these gRNAs to be highly suitable for the methods described herein, they need to be highly effective at introducing indels at a location in the KIF11 locus that would knock out or severely reduce gene function.

Each of these gRNAs was then tested to determine whether it could be used to knock-in a cassette comprising a portion of KIF11 and a cargo sequence encoding GFP into a terminal exon of the KIF11 locus of cells, in the process rescuing the lethal phenotype that would otherwise result by introducing RNP-induced indels into the coding region of this essential gene at a high frequency. Specifically, iPSC cells were contacted with an RNP containing AsCas12a (SEQ ID NO: 62), and RSQ33509, RSQ33510, or RSQ33511 (SEQ ID NOs: 155-157), along with a double stranded DNA donor template (dsDNA plasmid) designed to mediate HDR at each respective gRNA target binding site. The double stranded DNA donor templates included a knock-in cassette with a coding sequence for GFP (“Cargo”) in frame with and downstream (3′) of a codon optimized version of the final KIF11 exon coding sequence (exon 22) and a sequence encoding the P2A self-cleaving peptide (“P2A”), similar to the dsDNA plasmid described in Example 2 for GAPDH. The KIF11 sequence in the double stranded DNA donor templates (PLA1629, PLA1630, or PLA1631; comprising donor template SEQ ID NOs: 55-57) was codon optimized to prevent further binding by the accompanying guide RNA molecule (RSQ33509, RSQ33510, or RSQ33511; SEQ ID NOs: 155-157). The knock-in cassette also included 3′ UTR and polyA signal sequences downstream of the Cargo sequence. An RNP containing RSQ33509 (SEQ ID NO: 155) was administered with the PLA1629 plasmid (comprising donor template SEQ ID NO: 55); RSQ33510 (SEQ ID NO: 156) was administered with PLA1630 (comprising donor template SEQ ID NO: 56); and RSQ33511 (SEQ ID NO: 157) was administered with PLA1631 (comprising donor template SEQ ID NO: 57). Each particular dsDNA plasmid (PLA) contained a donor template with homology arms and a knock-in cassette designed to specifically encompass and render ineffective the particular gRNA target site following integration.

Flow cytometry was performed 7 days following nucleofection and was used to help determine to what extent each plasmid knock-in cassette was integrated successfully at its respective KIF11 target site. FIG. 17A shows that cells nucleofected with RNPs containing RSQ33509 (SEQ ID NO: 155) exhibited the greatest amount of GFP expression relative to cells nucleofected with the other RNPs targeting KIF11, suggesting that the GFP-encoding knock-in cassette integrated successfully in many of these cells. Cells nucleofected with RNPs containing RSQ33510 or RSQ33511 (SEQ ID NO: 156 or 157) also exhibited some GFP expression (FIG. 17A). FIG. 17B shows that use of the RNPs containing RSQ33509 (SEQ ID NO: 155) resulted in about 40% editing at 48 hours following transfection (the lower level possibly a result of significant cell death in the cell population at this time), correlating with the GFP expression levels depicted in FIG. 17A. Interestingly, FIG. 17B shows that use of RNPs containing RSQ33510 (SEQ ID NO: 156) resulted in about 90% observed editing rates, while RNPs containing RSQ33511 (SEQ ID NO: 157) resulted in about 65% observed editing rates, yet the GFP expression in cells transfected with these guides was relatively low when compared to RSQ33509 (SEQ ID NO: 155) transfected cells. These results suggest that the RSQ33510 or RSQ33511 (SEQ ID NO: 156 or 157) guides may not have been generating sufficiently deleterious indels in KIF11, allowing a high proportion cells to be viable despite high editing efficiencies, such that transfected cells were not dying in large enough numbers to allow for effective selection of transfected cells with successful cargo knocked in. Thus, although the RSQ33510 and RSQ33511 (SEQ ID NO: 156 or 157) gRNAs are highly specific for their KIF11 target sites (with minimal off-targets) and exhibit high editing levels, they may still not be suitable gRNAs for the selection mechanisms described herein as they may not induce toxic indels that result in sufficient malfunction of KIF11, which in turn would lead to cell death if homologous recombination of a rescue knock-in cassette does not occur. The percentage editing was measured two days following transfection and was determined by ICE assays (as described in Hsiau et al., August 2019).

### Example 12: Knock-In of Cargo at Essential Gene Loci Using a Viral Vector

The present example describes use of the gene editing methods described herein comprising viral vector transduction of a cell population.

The target cells described herein are collected from a donor subject or a subject in need to therapy (e.g., a patient). Following an appropriate sorting, culturing, and/or differentiation process, target cells are transduced with at least one AAV vector comprising a nucleotide sequence comprising a gRNA, a suitable nuclease, and/or a suitable rescue construct. Cells are sorted using flow cytometry to determine successful transduction, editing, integration, and/or expression events.

A population of hematopoietic stem cells are transduced with an AAV vector (e.g., AAV6) comprising GAPDH targeting RNP (including Cas12a of SEQ ID NO: 62 and gRNA RSQ22337 of SEQ ID NO: 95) and PLA1593 (comprising donor template SEQ ID NO: 44). Successful transduction, editing, knock-in cassette integration, and/or expression events are determined using flow cytometry, as described herein. Following AAV transduction, a large proportion of the cells are edited at the GAPDH locus by the RNP and have integrated the knock-in cassette via HDR. A population of hematopoietic stem cells are transduced with an AAV vector (e.g., AAV6) comprising TBP targeting RNP (including Cas12a of SEQ ID NO: 62 and gRNA RSQ33503 of SEQ ID NO: 149) and PLA1616 (comprising donor template SEQ ID NO: 49). Successful transduction, editing, integration, and/or expression events are determined using flow cytometry, as described herein. Following AAV transduction, a large proportion of the cells are edited at the TBP locus by the RNP and have integrated the knock-in cassette via HDR.

A population of T cells are transduced with an AAV vector (e.g., AAV6) comprising GAPDH targeting RNP (including Cas12a of SEQ ID NO: 62 and gRNA RSQ22337 of SEQ ID NO: 95) and PLA1593 (comprising donor template SEQ ID NO: 44). Successful transduction, editing, knock-in cassette integration, and/or expression events are determined using flow cytometry, as described herein. Following AAV transduction, a large proportion of the cells are edited at the GAPDH locus by the RNP and have integrated the knock-in cassette via HDR. A population of T cells are transduced with an AAV vector (e.g., AAV6) comprising TBP targeting RNP (including Cas12a of SEQ ID NO: 62 and gRNA RSQ33503 of SEQ ID NO: 149) and PLA1616 (comprising donor template SEQ ID NO: 49). Successful transduction, editing, integration, and/or expression events are determined using flow cytometry, as described herein. Following AAV transduction, a large proportion of the cells are edited at the TBP locus by the RNP and have integrated the knock-in cassette via HDR.

A population of NK cells are transduced with an AAV vector (e.g., AAV6) comprising GAPDH targeting RNP (including Cas12a of SEQ ID NO: 62 and gRNA RSQ22337 of SEQ ID NO: 95) and PLA1593 (comprising donor template SEQ ID NO: 44). Successful transduction, editing, knock-in cassette integration, and/or expression events are determined using flow cytometry, as described herein. Following AAV transduction, a large proportion of the cells are edited at the GAPDH locus by the RNP and have integrated the knock-in cassette via HDR. A population of NK cells are transduced with an AAV vector (e.g., AAV6) comprising TBP targeting RNP (including Cas12a of SEQ ID NO: 62 and gRNA RSQ33503 of SEQ ID NO: 149) and PLA1616 (comprising donor template SEQ ID NO: 49). Successful transduction, editing, knock-in cassette integration, and/or expression events are determined using flow cytometry, as described herein. Following AAV transduction, a large proportion of the cells are edited at the TBP locus by the RNP and have integrated the knock-in cassette via HDR.

A population of tumor-infiltrating lymphocytes (TILs) are transduced with an AAV vector (e.g., AAV6) comprising GAPDH targeting RNP (including Cas12a of SEQ ID NO: 62 and gRNA RSQ22337 of SEQ ID NO: 95) and PLA1593 (comprising donor template SEQ ID NO: 44). Successful transduction, editing, knock-in cassette integration, and/or expression events are determined using flow cytometry, as described herein. Following AAV transduction, a large proportion of the cells are edited at the GAPDH locus by the RNP and have integrated the knock-in cassette via HDR. A population of tumor-infiltrating lymphocytes (TILs) are transduced with an AAV vector (e.g., AAV6) comprising TBP targeting RNP (including Cas12a of SEQ ID NO: 62 and gRNA RSQ33503 of SEQ ID NO: 149) and PLA1616 (comprising donor template SEQ ID NO: 49). Successful transduction, editing, knock-in cassette integration, and/or expression events are determined using flow cytometry, as described herein. Following AAV transduction, a large proportion of the cells are edited at the TBP locus by the RNP and have integrated the knock-in cassette via HDR.

A population of neurons are transduced with an AAV vector (e.g., AAV6) comprising GAPDH targeting RNP (including Cas12a of SEQ ID NO: 62 and gRNA RSQ22337 of SEQ ID NO: 95) and PLA1593 (comprising donor template SEQ ID NO: 44). Successful transduction, editing, knock-in cassette integration, and/or expression events are determined using flow cytometry, as described herein. Following AAV transduction, a large proportion of the cells are edited at the GAPDH locus by the RNP and have integrated the knock-in cassette via HDR. A population of neurons are transduced with an AAV vector (e.g., AAV6) comprising TBP targeting RNP (including Cas12a of SEQ ID NO: 62 and gRNA RSQ33503 of SEQ ID NO: 149) and PLA1616 (comprising donor template SEQ ID NO: 49). Successful transduction, editing, knock-in cassette integration, and/or expression events are determined using flow cytometry, as described herein. Following AAV transduction, a large proportion of the cells are edited at the TBP locus by the RNP and have integrated the knock-in cassette via HDR.

### Example 13: Knock-In of Cargo at Essential Gene Loci Using a Viral Vector

The present example describes gene editing of populations of T cells using methods described herein comprising viral vector transduction of populations of T cells. The methods described herein can be applied to other cell types as well, such as other immune cells.

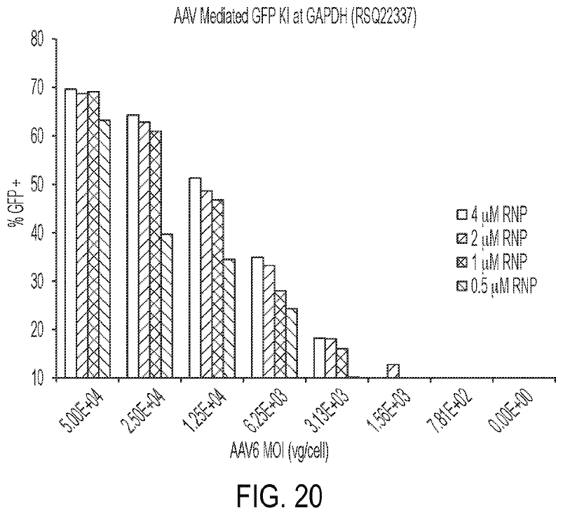

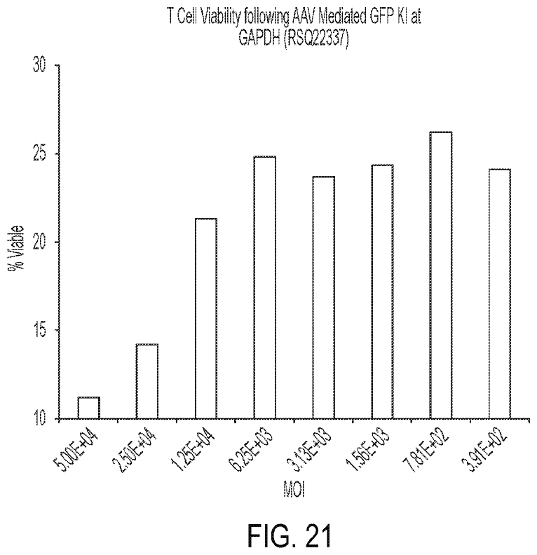

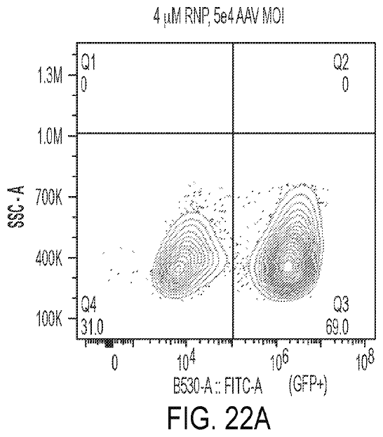

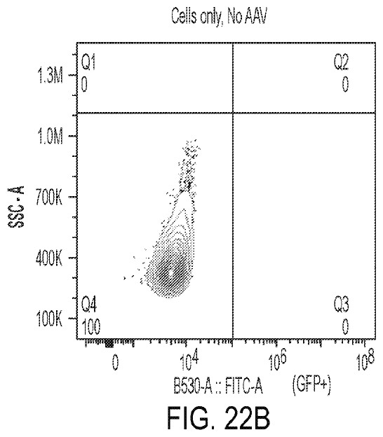

T cells were thawed in a bead bath as known in the art and were removed from the bath on day two. Cells were electroporated on day four after thawing, in brief 250,000 T cells per well in a Lonza 96-well cuvette were suspended in buffer P2 and electroporated using pulse code CA-137 with varying concentrations of RNP comprising gRNA RSQ22337 (SEQ ID NO: 95) and Cas12a (SEQ ID NO: 62) targeting the GAPDH gene (404 RNP, 204 RNP, 1 μM RNP, or 0.5 μM RNP). Appropriate media was added to cells immediately after electroporation and cells were allowed to recover for 15 minutes. AAV6 viral particles comprising a donor plasmid construct containing a knock-in cassette with a GFP cargo were then added to T cells at varying multiplicity of infection (MOI) concentrations (5E4, 2.5E4, 1.25E4, 6.25E3, 3.13E3, 1.56E3, or 7.81E2). The donor plasmid was designed as described in Example 2, with a 5′ codon-optimized coding portion of GAPDH exon 9 optimized to prevent further binding of the gRNA targeting domain sequence of the guide RNA (RSQ22337)), an in-frame sequence encoding the P2A self-cleaving peptide (“P2A”), an in-frame coding sequence for GFP (“Cargo”), a stop codon and polyA signal sequence. T cells were split two days later, and then every 48 hours until they were analyzed by flow cytometry. T cells were sorted using flow cytometry seven days post electroporation to determine successful transduction, transformation, editing, knock-in cassette integration, and/or expression events (see FIG. 20, FIG. 21, FIG. 22A, and FIG. 22B). As shown in FIG. 20, populations of T cells were transduced with 4 μM RNP, 2 μM RNP, 1 μM RNP, or 0.5 μM RNP, at various AAV6 multiplicity of infection (MOI) (5E4, 2.5E4, 1.25E4, 6.25E3, 3.13E3, 1.56E3, or 7.81E2). High proportions of GFP integration at the GAPDH gene were observed in T cell populations transduced/transformed with all RNP concentrations at 5E4 AAV6 MOI and were observed with RNP concentrations greater than 1 μM when cells were transduced with AAV6 MOI as low as 1.25E4 (see FIGS. 20 and 22A). Control experiments with no AAV transduction resulted in T cell populations that displayed no GFP integration events (see FIG. 22B). T cell viability was measured four days after cells were transformed with RNPs and AAV6 at various MOI (FIG. 21).

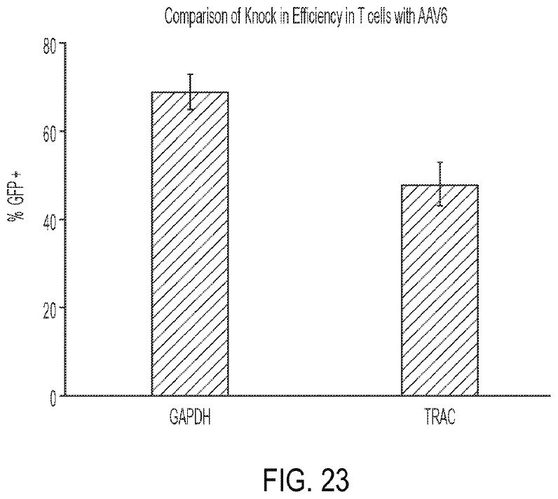

Furthermore, knock-in efficiencies using methods described herein were compared to optimized versions of methods known in the art. In brief, T cell populations were transduced with AAV6 vector comprising a donor template suitable for knock-in of GFP at the GAPDH gene as described herein, and were transformed with gRNA RSQ22337 (SEQ ID NO: 95) and Cas12a (SEQ ID NO: 62) as described above; alternatively, T cell populations were subject to highly optimized GFP knock-in at the TRAC locus using AAV6 vector transduction (see e.g., Vakulskas et al. A high-fidelity Cas9 mutant delivered as a ribonucleoprotein complex enables efficient gene editing in human hematopoietic stem and progenitor cells. Nat Med. 2018; 24(8):1216-1224). Flow cytometry was utilized to measure knock-in efficiency (determined by percentage of T cell population expressing GFP, measured 7 days post-electroporation). Knock-in rates at the TRAC locus were high (˜50%) when compared to publicly described integration frequencies for similar methodologies, however, knock-in efficiency at the GAPDH gene using methods described herein facilitated by AAV6 transduction were significantly (p=0.0022 using unpaired t-test) higher (˜68%) (see FIG. 23). The same RNP concentration, AAV6 MOI, and homology arm lengths were utilized in both experiments, averaged results from three independent biological replicates are shown (see FIG. 23). Thus, the methods described herein can be used to isolate a population of modified cells, such as immune cells like T cells, that highly express a gene of interest relative to other gene knock-in methods.

### Example 14: CD16 Knock-In iPSCs Give Rise to Edited iNKs with Enhanced Function

The present example describes use of gene editing methods described herein to create modified immune cells suitable for killing cancer cells.

PSCs were edited using the exemplary system illustrated in FIGS. 3A, 3B, and 3C, and described in Example 2. In brief, the GAPDH gene was targeted in iPSCs using AsCpf1 (SEQ ID NO: 62), and a guide RNA (RSQ22337) (SEQ ID NO: 95), resulting in a double-strand break towards the 5′ end of the last exon of GAPDH (exon 9). The CRISPR/Cas nuclease and guide RNA were introduced into cells by nucleofection (electroporation) of a ribonucleoprotein (RNP) according to known methods. The cells were also contacted with a double stranded DNA donor template (dsDNA plasmid, comprising donor template SEQ ID NO: 205) that included a donor template comprising in 5′-to-3′ order, a 5′ homology arm approximately 500 bp in length (comprising a 3′ portion of exon 8, intron 8, and a 5′ codon-optimized coding portion of exon 9 optimized to prevent further binding of the gRNA targeting domain sequence of the guide RNA (RSQ22337)), an in-frame sequence encoding the P2A self-cleaving peptide (“P2A”), an in-frame coding sequence for CD16 (“Cargo”) (a non-cleavable CD 16; SEQ ID NO: 165), a stop codon and polyA signal sequence, and a 3′ homology arm approximately 500 bp in length (comprising a coding portion of exon 9 including a stop codon, the 3′ non-coding exonic region of exon 9, and a portion of the downstream intergenic sequence) (as shown in FIG. 3B).

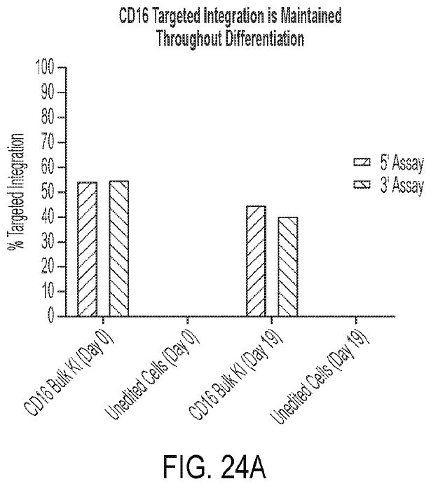

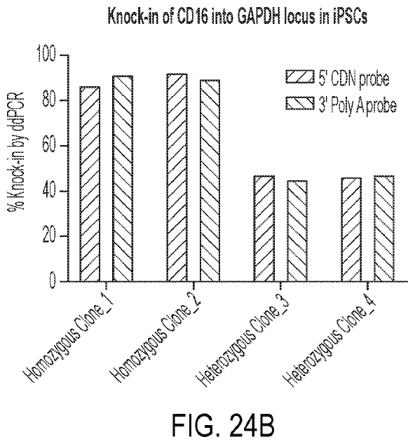

The cargo gene CD16 was successfully integrated into the GAPDH gene of iPSCs at high efficiencies using the selection systems described herein. FIG. 24A shows the efficiency of CD16-encoding “cargo” integration in the GAPDH gene at 0 days post-electroporation and at 19 days post-electroporation in iPSCs transformed with RNPs at a concentration of 4 μM and the dsDNA plasmid encoding CD16, or in “unedited cells” that were not transformed with the dsDNA plasmid. Knock-in was measured in bulk edited CD16 KI cells using ddPCR targeting the 5′ or 3′ position of the knock-in “cargo” using a primer in the 5′ of the gRNA target site or a primer in the 3′ of the site in the poly A region, increasing the reliability of the result. As shown in FIG. 24A, CD16 was stably knocked-in and present in bulk edited cell populations more than two weeks following electroporation and targeted integration of the knock-in cassette.

From bulk edited cell populations, single cells were propagated to homogenize genotypes. Shown in FIG. 24B are four edited cell populations: homozygous clone 1, homozygous clone 2, heterozygous clone 3, and heterozygous clone 4. The homozygous clones contained two alleles of the GAPDH gene that comprised CD16 knock-in, while heterozygous clones contained one allele of the GAPDH gene that comprised CD16 knock-in (measured using ddPCR of the 5′ and 3′ positions of the knock-in cargo).

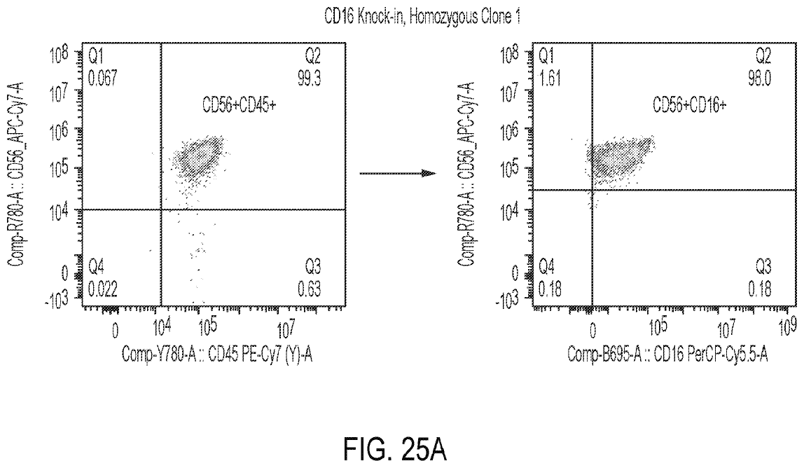

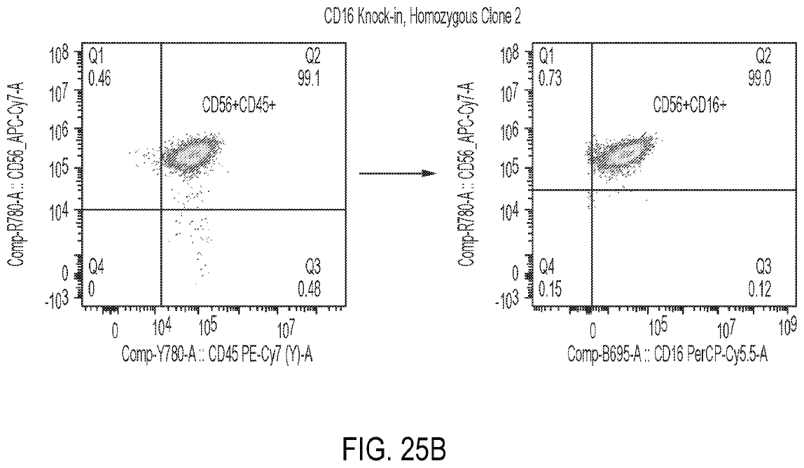

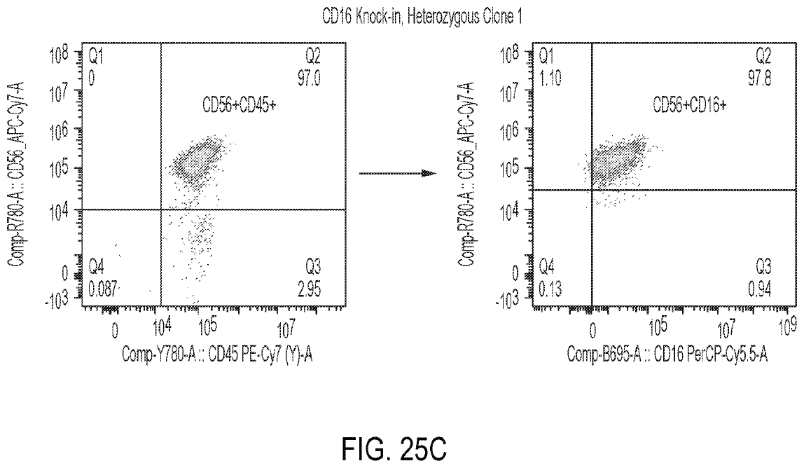

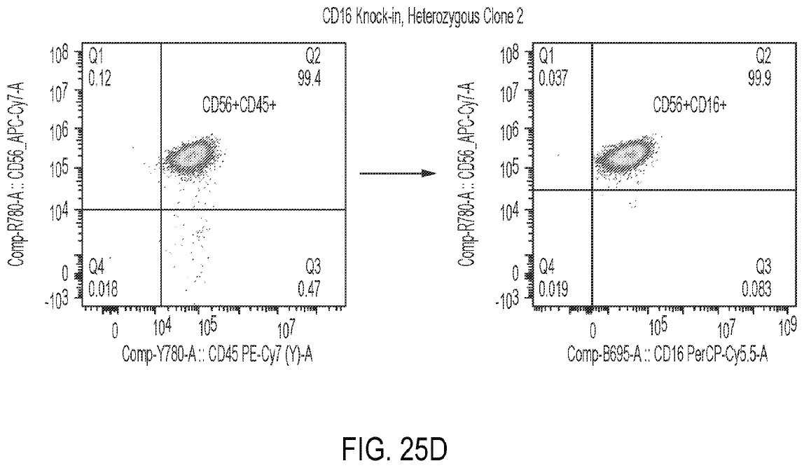

Following confirmation of CD16-encoding “cargo” integration at the GAPDH gene, homogenized cell lines were differentiated into Natural Killer (NK) immune cells using spin embryoid body methods as known in the art. In brief, iPSCs were placed in an ultra-low attachment 96-well plate at 5,000 to 6,000 cells per well in order to form embryoid bodies (EBs). On day 11 EBs were transferred to a flask where they remain for the remainder of the experiment (see Ye Li et al., Cell Stem Cell. 2018 Aug. 2; 23(2): 181-192.e5). At day 32 of the differentiation process, cells were analyzed using flow cytometry methods known in the art. Following standard control gating experiments (see Ye Li et al., Cell Stem Cell. 2018 Aug. 2; 23(2): 181-192.e5), the differentiation process was analyzed using expression of markers CD56 and CD45, following this, co-expression of markers CD56 and CD16 was measured. As shown in FIG. 25A-25D, in general, cells that were positive for CD56 expression were also positive for CD16 expression (98%, 99%, 97.8%, and 99.9% respectively), indicating that both homozygous and heterozygous TI clones had stable and robust CD16 expression levels.

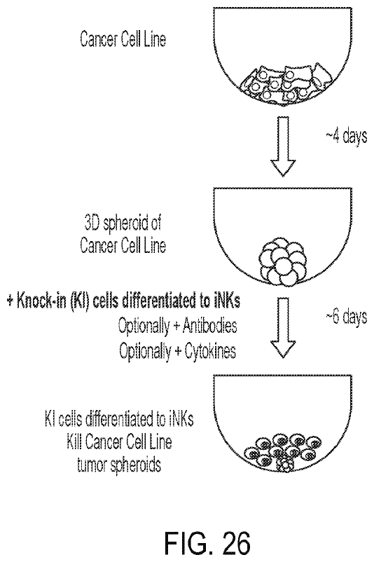

These differentiated iNK cells comprising knock-in of the gene of interest (CD16) at the GAPDH gene were then subject to challenge by various cancer cell lines to determine their cytotoxic capacity. An exemplary 3D solid tumor killing assay is depicted in FIG. 26. In brief, spheroids were formed by seeding 5,000 NucLight Red labeled SK-OV-3 cells in 96 well ultra-low attachment plates. Spheroids were incubated at 37° C. before addition of effector cells (at different E:T ratios) and any optional agents (e.g., cytokines, antibodies, etc.), spheroids were subsequently imaged every 2 hours using the Incucyte S3 system for up to 600 hours. Data shown are normalized to the red object intensity at time of effector addition. Normalization of spheroid curves maintains the same efficacy patterns observed in non-normalized data. Using this assay, the cytotoxicity of iNKs differentiated from iPSCs comprising knock-in of CD16 at the GAPDH gene was measured.

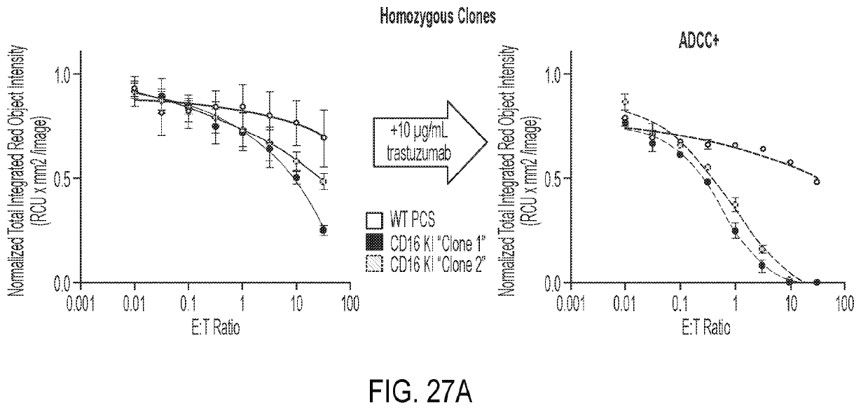

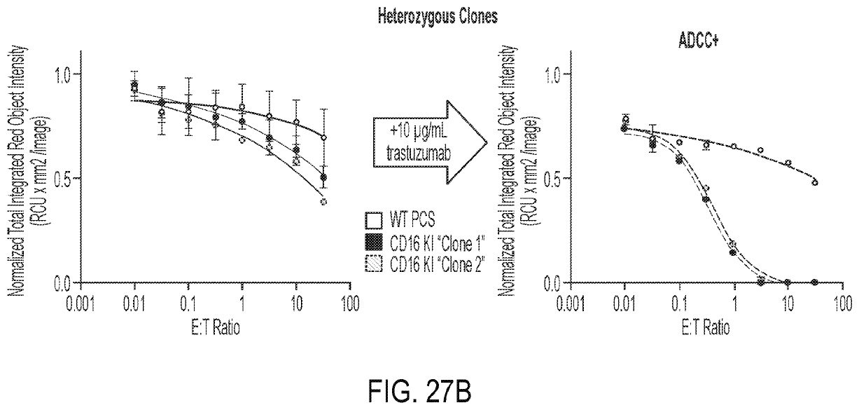

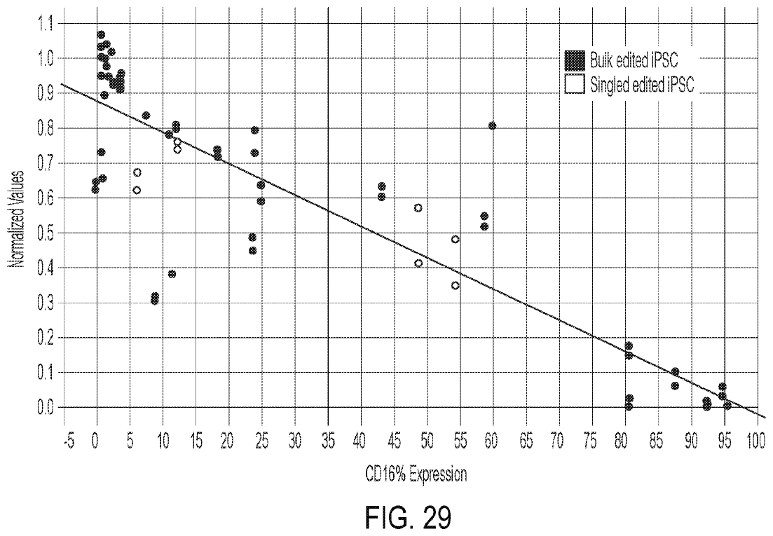

As shown in FIGS. 27A and 27B, both homozygous edited iNK lines and both heterozygous edited iNK lines comprising CD16 knocked-in at the GAPDH gene were capable of reducing the size of SK-OV-3 spheroids more effectively than unedited iNK control cells (WT PCS) or control cells with GFP knocked-in to the GAPDH gene (WT GFP KI) (averaged data from 2 assays). The edited homozygous and heterozygous iNK cells comprising CD16 at GAPDH also reduced the size of SK-OV-3 spheroids more effectively than control cells with GFP knocked-in to the GAPDH gene (data not shown). Introduction of 10 μg/mL of the antibody trastuzumab greatly enhanced the killing capacity of the CD16 KI iNKs when compared to control cells, likely as a function of increased antibody dependent cellular cytotoxicity (ADCC) due to increased FcγRIII (CD16) expression levels. The results of a number of solid tumor killing assays were plotted against the CD16 expression levels of CD16 KI edited iNKs (derived from bulk edited iPSCs or singled edited iPSCs). At an E:T ratio of 3.16:1, there is a correlation shown between the percentage of a cell population expressing CD16, and the amount of cell killing that occurred (see FIG. 29).

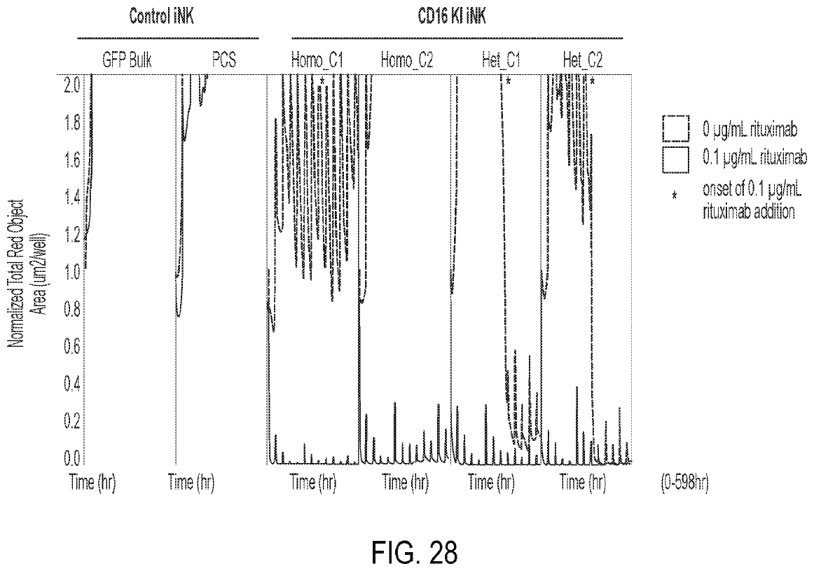

To further elucidate the functionality of the edited iNKs, the cells were subjected to repeated exposure to tumor cells, and the ability of the edited iNKs to kill tumor targets repeatedly over a multiday period was analyzed in an in vitro serial killing assay. Results of this experiment are depicted in FIG. 28. At day 0 of the assay, 10×10⁶ Raji tumor cells (a lymphoblast-like cell line of hematopoietic origin) and 2×10⁵ iNKs were plated in each well of a 96-well plate in the presence or absence of 0.1 μg/mL of the antibody rituximab. At approximately 48 hour intervals, a bolus of 5×10³ Raji tumor cells was added to re-challenge the iNK population. As shown in FIG. 28, the edited iNK cells (CD16 KI iNK heterozygous or homozygous) exhibited continued killing of Raji cells after multiple challenges with Raji tumor cells (up to 598 hours), whereas unedited iNK cells were limited in their serial killing effect. The data show that iNK cells comprising homozygous or heterozygous CD16 KI at GAPDH results in prolonged and enhanced tumor cell killing. Furthermore, the efficacy of heterozygous CD16 KI iNKs highlights the potential for biallelic insertion of two different knock-in cassettes, e.g., comprising CD16 in one allele and a different gene of interest in the other allele of a suitable essential gene (e.g., GAPDH, TBP, KIF11, etc.).

### Example 15: Knock-In of Immunologically Relevant Sequences at a Suitable Essential Gene Locus (Monocistronic or Bicistronic)

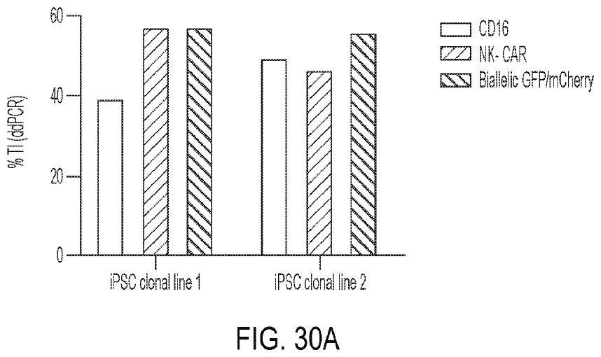

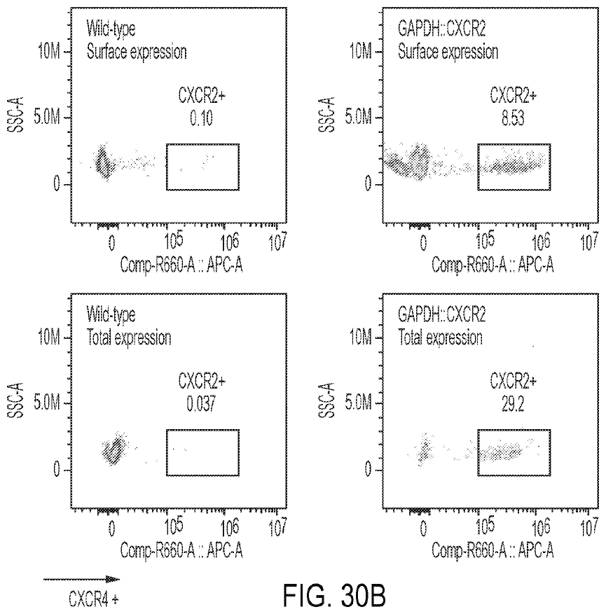

Positive targeted integration events at the GAPDH gene and cellular phenotypes were noted for integration of GFP, CD47, or CD16 as described above in Example 2 and Example 15. Additional or alternative cargo sequences may be incorporated into the GAPDH gene or other suitable essential genes as described herein with high integration rates. The essential gene GAPDH was targeted in iPSC cells using an RNP containing AsCpf1 (SEQ ID NO: 62) and a guide RNA (RSQ22337; SEQ ID NO: 95), resulting in a double-strand break towards the 5′ end of the last exon of GAPDH (exon 9), as described in Example 2. A donor plasmid containing a knock-in cassette with the cargo of interest was also electroporated with the RNP. As shown in FIG. 30A, the targeted integration (TI) rates at the GAPDH gene for cargos such as a) CD16, b) a CAR suitable for expression in NK cells, or c) biallelic GFP/mCherry, were all greater than 40% when assayed in two independent iPSC clonal lines when measured using ddPCR. As shown in FIG. 30B, the targeted TI rates at the GAPDH gene for a CXCR2 cargo was at least 29.2% of bulk edited iPSCs (expression determined using flow cytometry), while surface expression of CXCR2 was observed in approximately 8.5% of the bulk edited iPSCs (expression determined using flow cytometry). By contrast, unedited iPSCs very small amounts of CXCR2 (approximately 1%) by flow cytometry (data not shown).

An exemplary ddPCR experiment was used to measure the targeted integration (TI) rates as follows. In brief, TI was measured using a universal set of primers that captures both the 5′ homology arm and 3′ polyA tail for the GAPDH terminal exon region, and can detect cargos independent of the particular sequence of the specific cargo. The 5′ CDN primer and 3′ PolyA primer and FAM fluorophore probes are made in combination. An appropriate reference gene probe is a TTC5 HEX probe. For the reaction, probes, genomic DNA, BioRad master mix, and 2×control buffer were mixed together in ratios consistent with manufacturer recommendations. First, genomic DNA was placed in the BioRad 96 well plate (9.2 μl total genomic DNA+water), next, master mix with primer probes sets (13.8 μl per well) were added. Water controls comprised a 5′ primer probe set master mix in one well, and a 3′ primer probe set master mix in a different well. For blank well controls, a 50/50 mix of 2×control buffer and water (25 μl total) was added. The auto droplet generator was then prepared and run. Once droplets were generated, the ddPCR plates were sealed at 180° C. and then placed in a thermocycler for amplification. 5′ CDN primer: CATCGCATTGTCTGAGTAGGTGTC (SEQ ID NO: 219), 3′ PolyA primer: TGCCCACAGAATAGCTTCTTCC (SEQ ID NO: 220), FAM probe: TCCCCTCCTCACAGTTGCCA (SEQ ID NO: 221), TTC5 reference gene forward primer: GGAGAAAGTGTCCAGGCATAAG (SEQ ID NO: 222), TTC5 reference gene reverse primer: CTCCATCCCACTATGACCATTC, (SEQ ID NO:223), TTC5 FAM probe: AGTTTGTGTCAGGATGGGTGGT (SEQ ID NO: 224).

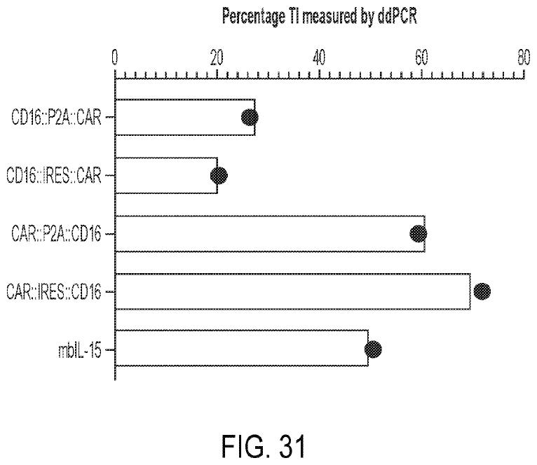

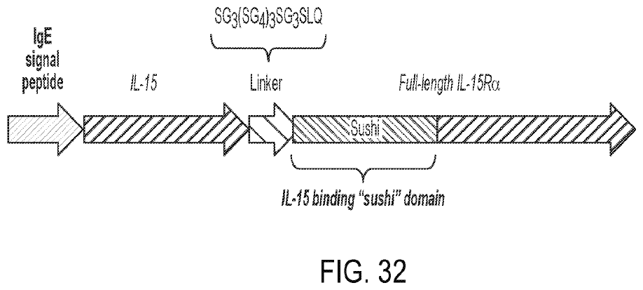

Next, the cargo integration and selection methods described herein were tested using a number of bicistronic knock-in cassettes that contained CD16 and an NK suitable CAR in different 5′-to-3′ orders (e.g., CD16 followed by the CAR, or the CAR followed by CD16) and separated by a P2A or IRES sequence. The essential gene GAPDH was targeted in iPSC cells using an RNP containing AsCpf1 (AsCas12a, (SEQ ID NO: 62)) and a guide RNA (RSQ22337; SEQ ID NO: 95), resulting in a double-strand break towards the 5′ end of the last exon of GAPDH (exon 9), as described in Example 2. A donor plasmid containing each of the knock-in cassettes depicted in FIG. 31 was also electroporated with the RNP. As shown in FIG. 31, the TI rates for the bicistronic constructs comprising CD16 and the NK suitable CAR ranged from 20-70% when measured in the bulk edited cells using ddPCR at day 0 post-transformation. In addition, a membrane bound IL-15 (mbIL-15) cargo gene (a fusion comprising IL-15 linked to a Sushi domain and a full-length IL-15Rα, as depicted in FIG. 32) was also knocked into the GAPDH locus using RNPs comprising (RSQ22337) and Cas12a at a concentration of 4 μM and the dsDNA plasmid encoding mbIL-15 at 5 (PLA1632; comprising donor template SEQ ID NO: 45) to determine if additional genes of interest could be integrated into an essential gene at high levels within a population of edited cells. FIG. 31 shows that the mbIL-15 cargo was knocked into the GAPDH locus at a percentage TI of greater than 50% as measured by ddPCR (day 0 post-transformation). Thus, the methods described herein can be used to isolate populations of edited cells, such as iPSCs, that have very high levels of a gene of interest knocked into an essential gene locus, such as GAPDH.

### Example 16: IL-15 and/or IL-15/IL15-Rα Knock-In iPSCs Give Rise to Edited iNKs with Enhanced Function

The present example describes use of gene editing methods described herein to create modified immune cells suitable for cancer cell killing.

PSCs were edited using the exemplary system illustrated in FIGS. 3A, 3B, and 3C, and described in Example 2. In brief, the GAPDH gene was targeted in iPSCs using RNPs containing AsCpf1 (AsCas12a, SEQ ID NO: 62), and a guide RNA (RSQ22337; SEQ ID NO: 95), resulting in a double-strand break towards the 5′ end of the last exon of GAPDH (exon 9). The CRISPR/Cas nuclease and guide RNA were introduced into cells by nucleofection (electroporation) of a ribonucleoprotein (RNP) according to known methods. The cells were also contacted with a double stranded DNA donor template (dsDNA plasmid, PLA) that included a donor template comprising in 5′-to-3′ order, a 5′ homology arm approximately 500 bp in length (comprising a 3′ portion of exon 8, intron 8, and a 5′ codon-optimized coding portion of exon 9 optimized to prevent further binding of the gRNA targeting domain sequence of the guide RNA (RSQ22337)), an in-frame sequence encoding the P2A self-cleaving peptide (“P2A”), an in-frame coding sequence for mbIL-15 as shown in FIG. 32 (“Cargo”) (SEQ ID NO: 172), a stop codon and polyA signal sequence, and a 3′ homology arm approximately 500 bp in length (comprising a coding portion of exon 9 including a stop codon, the 3′ non-coding exonic region of exon 9, and a portion of the downstream intergenic sequence) (as shown in FIG. 3B). The 5′ and 3′ homology arms flanking the cargo coding sequence of the donor template were designed to correspond to sequences located on either side of the endogenous stop codon in the genome of the cell.

The cargo gene mbIL-15 (as shown in FIG. 32) was successfully integrated into the GAPDH gene of iPSCs at high efficiencies using the selection systems described herein (see Example 15). FIG. 31 shows the efficiency of the mbIL-15-encoding “cargo” in GAPDH at 0 days post-electroporation in iPSCs transformed with RNPs comprising (RSQ22337) and Cas12a at a concentration of 4 μM and the dsDNA plasmid encoding mbIL-15 at 5 (PLA1632; comprising donor template SEQ ID NO: 45). Genomic DNA was extracted approximately seven days post nucleofection. After genomic DNA extraction ddPCR was performed.

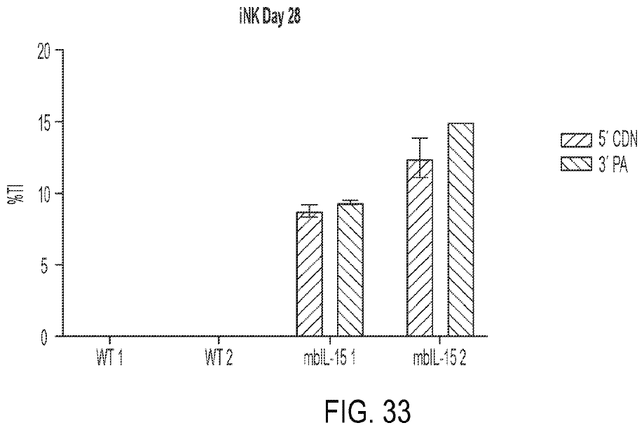

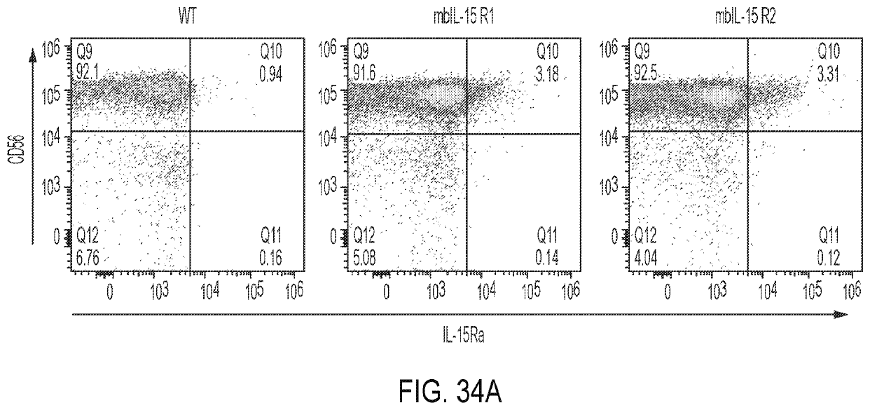

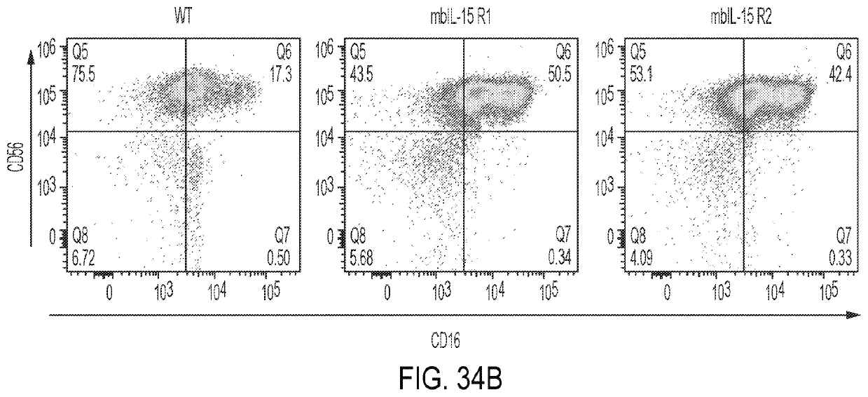

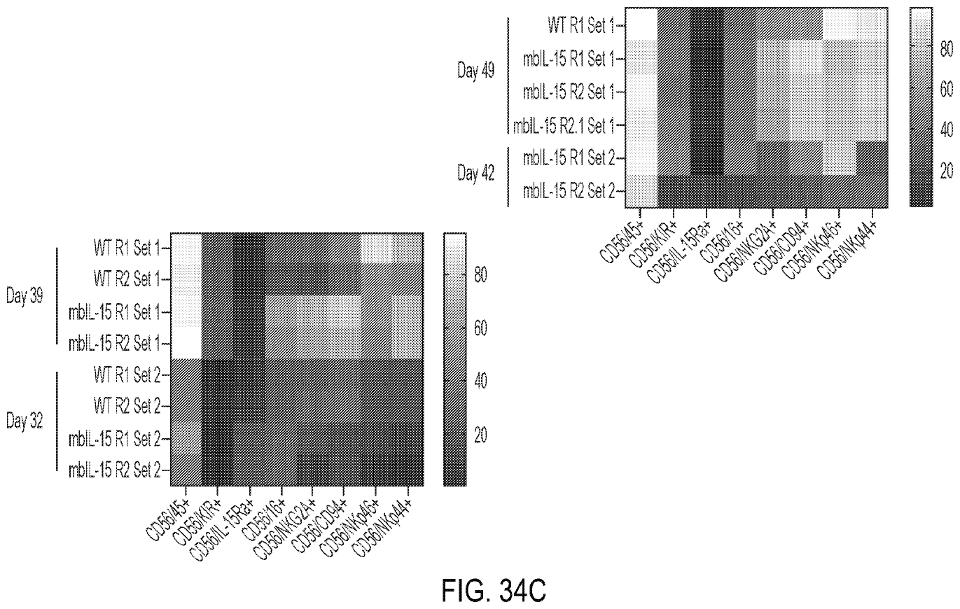

Two separate populations of the bulk edited mbIL-15 KI iPSC cells were then differentiated into iNK cells and the TI rates were measured using ddPCR at day 28 of the iNK differentiation process. FIG. 33 shows that TI integrate rates for these edited iNK cell populations ranged from 10-15%. While the TI rates in the iNK populations decreased when compared to the TI at day 0 post-electroporation of iPSCs, the TI integration levels within these cell populations remained significant. At day 32 post-differentiation initiation, flow cytometry was conducted to determine the proportion of cells expressing CD56 and exogenous IL-15Rα in these edited iNK cell populations (see FIG. 34A). The CD56 and CD16 co-expression levels were also determined in these edited iNK cell populations (see FIG. 34B). The bulk edited mbIL-15 KI cell populations were also analyzed for markers of differentiation by flow cytometry on day 32, day 39, day 42, and day 49 post-differentiation initiation (see FIG. 34C).

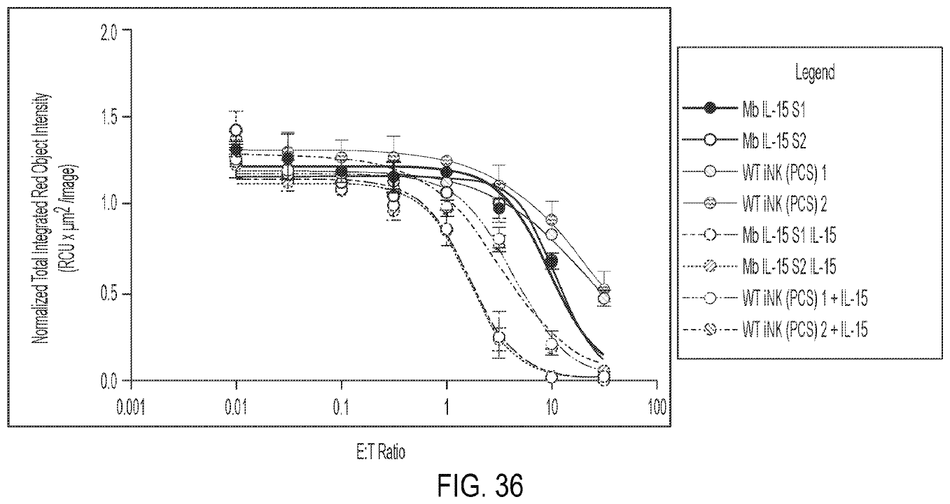

At day 39 following the initiation of differentiation from the edited iPSCs into iNKs, cells were challenged in 3D spheroid killing assays as described in Example 14 and depicted in FIG. 26. Using this assay, the cytotoxicity of iNKs differentiated from iPSCs comprising knock-in of mbIL-15 at the GAPDH gene was measured (see FIG. 36). Cells were tested in the presence or absence of 5 ng/mL exogenous IL-15. As shown in Table 15 and FIG. 36, mbIL-15 KI iNK cells (Mb IL-15 S1 and Mb IL-15 S2 populations) exhibited more efficient tumor cell killing when compared to unedited parental cells differentiated into iNKs (“WT” PCS, 1 and 2). Of note, mbIL-15 KI iNK cells exhibited better tumor cell killing in the absence of exogenous IL-15 relative to WT iNK cells in the absence of endogenous IL-15 at lower E:T ratios. The mbIL-15 KI iNK cells also exhibited better tumor cell killing in the presence of low concentrations of exogenous IL-15 (5 ng/mL) when compared to unedited WT iNK cells in the presence of the same concentration of exogenous IL-15.

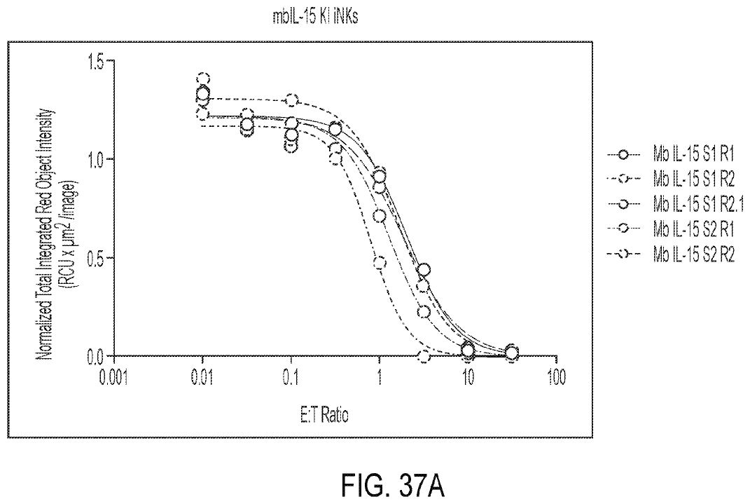

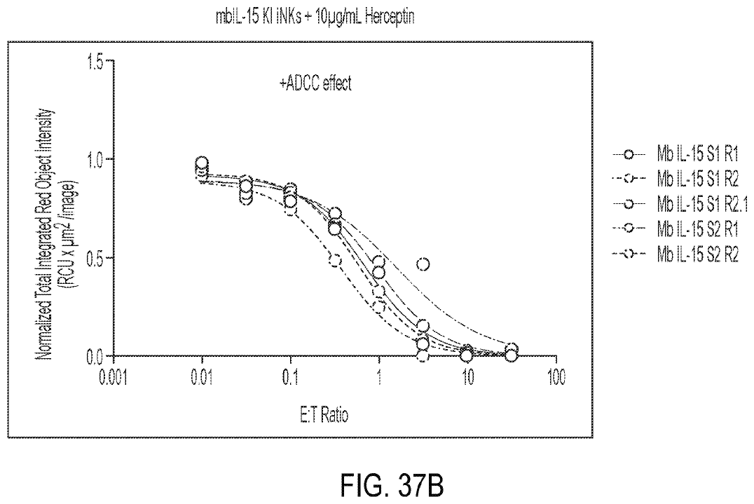

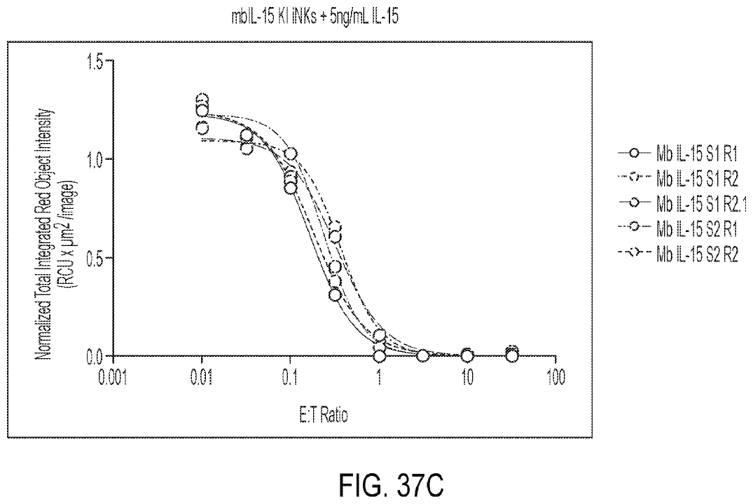

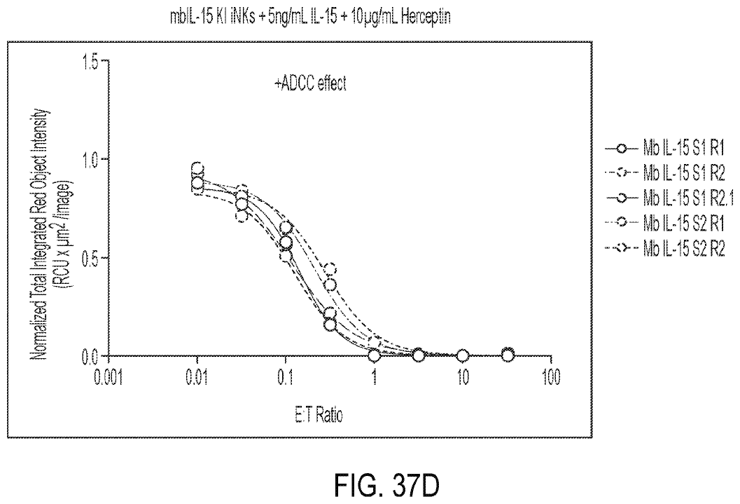

In addition, mbIL-15 KI iNK cells at later stages of differentiation (day 63 post-differentiation initiation for Set 1 (S1) and day 56 post-differentiation initiation for Set 2 (S2)) were also challenged in 3D spheroid killing assays as described above. Cells were tested in the presence or absence of 10 μg/ml Herceptin and/or 5 ng/mL exogenous IL-15. As shown in Table 16 and FIG. 37A-37D, mbIL-15 KI iNK cells exhibited high tumor cell killing efficiency, particularly when coupled with antibody therapy. At day 63, all mbIL-15 KI iNK cells did not express detectable levels of IL-15Ra; at Day 56, only one mbIL-15 KI iNK cell line (Mb IL-15 S2 R2) expressed detectable levels of IL-15Ra (data not shown).

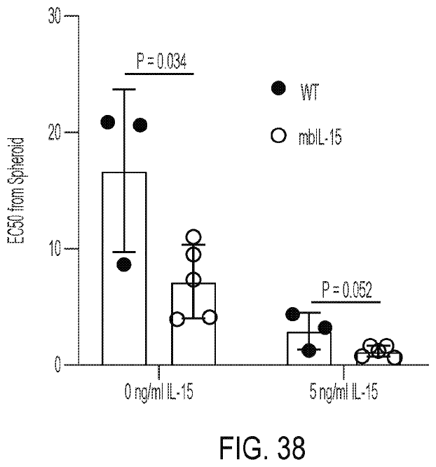

The cumulative results of certain 3D spheroid killing assays for mbIL-15 KI iNKs and control WT iNK cells is depicted in FIG. 38. Two independent bulk edited populations of iPSCs (Set 1 (S1) and Set 2 (S2)) comprising mbIL-15 knock-in at the GAPDH gene were differentiated into iNK cells (day 39 and 49 of iPSC differentiation for Set 1, and day 42 of iPSC differentiation for Set 2) These iNK cells significantly reduced tumor cell spheroid size when compared to differentiated WT parental cell iNKs in the absence of exogenous IL-15 (P=0.034, +/−standard deviation, unpaired t-test). The differentiated knock-in mbIL-15 iNK cells also trended towards significant reduction of tumor cell spheroid size when compared to differentiated WT parental cells in the presence of 5 ng/mL exogenous IL-15 (P=0.052, +/−standard deviation, unpaired t-test). These results show that populations of iNK cells comprising mbIL-15 knock-in at the GAPDH locus using the methods described herein perform better in killing tumor cells in the absence of exogenously added IL-15 compared to populations of unedited iNK cells.

TABLE 15
mbIL-15 KI iNK 3D spheroid killing with IL-15

| | EC50 with 0 ng/mL | EC50 with 5 ng/mL |
|---|---|---|
| Cell Line | IL-15 | IL-15 |
| Mb IL-15 S1 | 9.575 | 1.648 |
| Mb-IL-15 S2 | 11.05 | 1.646 |
| WT iNK (PCS) 1 | 20.71 | 4.378 |
| WT iNK (PCS) 2 | 20.99 | 3.213 |

TABLE 16
mbIL-15 KI iNK 3D spheroid killing with Herceptin and/or IL-15

| | | | EC50 with | EC50 with |
|---|---|---|---|---|
| | | | 5 ng/mL | 5 ng/mL |
| | EC50 with | EC50 with | IL-15 and | IL-15 and |
| | 0 μg/mL | 10 μg/mL | 0 μg/mL | 10 μg/mL |
| Cell Line | Herceptin | Herceptin | Herceptin | Herceptin |
| Mb IL-15 Set1 Rep1 | 2.055 | 0.6936 | 0.16515 | 0.1423 |
| Mb IL-15 Set1 Rep2 | 1.701 | 0.5903 | 0.1794 | 0.1247 |
| Mb IL-15 Set1 Rep2.1 | 1.848 | 0.9570 | 0.3187 | 0.1153 |
| Mb IL-15 Set2 Rep1 | 1.291 | 1.589 | 0.2339 | 0.2096 |
| Mb IL-15 Set2 Rep2 | 0.8026 | 0.3783 | 0.3605 | 0.2778 |

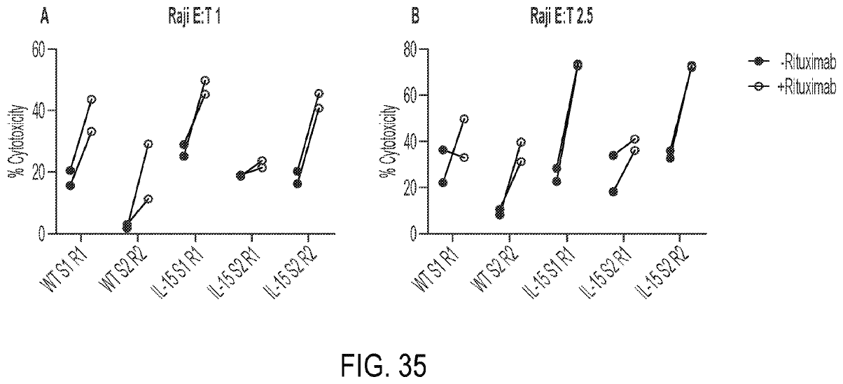

In addition, the mbIL-15 KI iNK cells at later stages of differentiation (day 63 post-differentiation initiation for Set 1 (51) and day 56 post-differentiation initiation for Set 2 (S2)) were also challenged with hematological cancer cells (e.g., Raji cells). Two biological replicate populations of mbIL-15 KI NK cells (S1 and S2) were tested in the presence or absence of 10 μg/ml rituximab. As shown in FIG. 35, mbIL-15 KI iNK cells exhibited high tumor cell killing efficiency, particularly when coupled with antibody therapy. This killing capacity of these cells is significant, as Raji cells are naturally resistant to NK cells, but the mbIL-15 KI iNK cells in combination with antibody were able to find and kill these cells.

### Example 17: Knock-In of Multicistronic CD16, IL-15, and/or IL-15Rα Sequences at a Suitable Essential Gene Loci

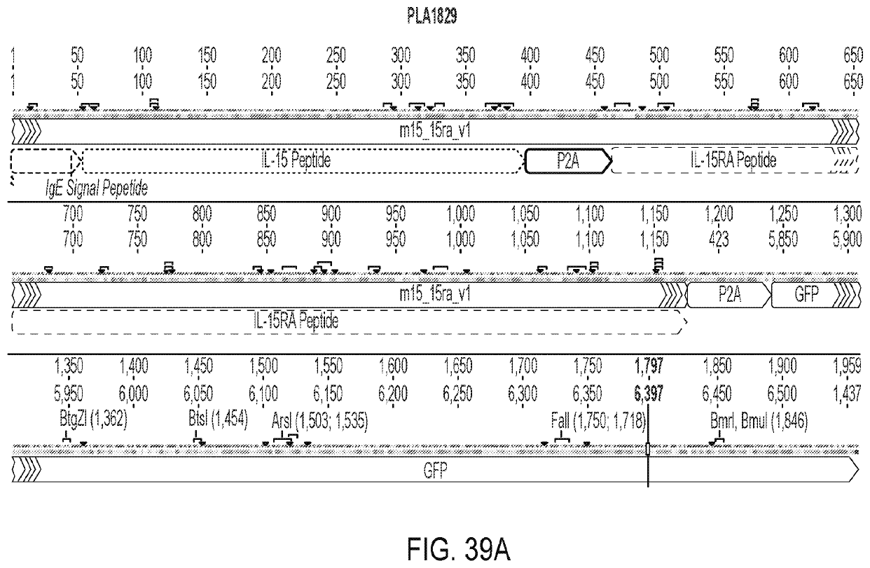

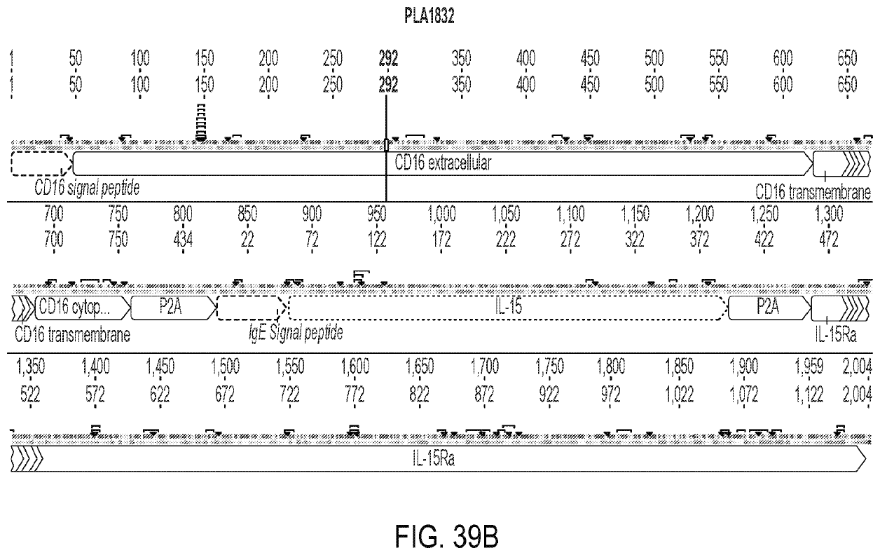

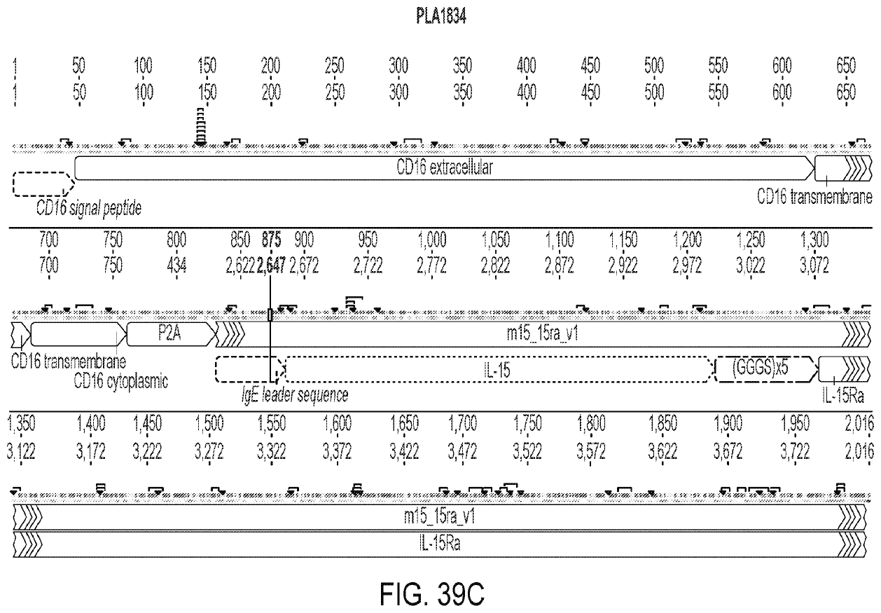

As described above in Example 2, genes of interest (GOI) may be integrated as a cargo sequence into suitable essential gene loci using methods described herein. In certain embodiments, multiple GOIs may be combined into a bicistronic or multicistronic knock-in cargo sequence. FIG. 39A depicts a portion of PLA1829 (comprising donor template SEQ ID NO: 208) comprising a bicistronic knock-in cargo sequence that was utilized for targeted integration at the GAPDH gene comprising an IL-15 peptide sequence, an IL-15Rα peptide sequence, and a GFP peptide sequence (SEQ ID NOs: 187, 189, and 195 respectively). Each of these peptide sequences were separate by a P2A sequence. Depicted in FIG. 39B is a portion of PLA1832 (comprising donor template SEQ ID NO: 209) comprising a multicistronic knock-in cargo sequence that was utilized for targeted integration at the GAPDH gene comprising a CD16 peptide sequence, an IL-15 peptide sequence, and an IL-15Rα peptide sequence (SEQ ID NO: 184, 187, and 189 respectively). Each of these peptide sequences were separate by a P2A sequence. Depicted in FIG. 39C is a portion of PLA1834 (comprising donor template SEQ ID NO: 212) comprising a bicistronic knock-in cargo sequence that was utilized for targeted integration at the GAPDH gene comprising a CD16 peptide sequence, and an mbIL-15 peptide sequence (an IL-15 sequence fused to an IL-15Rα sequence as depicted in FIG. 32) (SEQ ID NOs: 184 and 190 respectively) separated by a P2A sequence.

The knock-in cargo sequences described in FIG. 39A-39C are comprised within Plasmids 1829, 1832, and 1834 respectively (comprising donor template SEQ ID NO: 208, 209, and 212). PSCs were edited using the exemplary system illustrated in FIGS. 3A, 3B, and 3C, and described in Example 2. In brief, the GAPDH gene was targeted in iPSCs using AsCpf1 (AsCas12a (SEQ ID NO: 62)) and a guide RNA (RSQ22337 (SEQ ID NO: 95)), resulting in a double-strand break towards the 5′ end of the last exon of GAPDH (exon 9). The CRISPR/Cas nuclease and guide RNA were introduced by nucleofection (electroporation) of a ribonucleoprotein (RNP) according to known methods. The cells were also contacted with a double stranded DNA donor template (dsDNA plasmid (PLA1829, PLA1832, or PLA1834 respectively)) that included a donor template (SEQ ID NOs: 208, 209, and 212) comprising in 5′-to-3′ order, a 5′ homology arm approximately 500 bp in length (comprising a 3′ portion of exon 8, intron 8, and a 5′ codon-optimized coding portion of exon 9 optimized to prevent further binding of the gRNA targeting domain sequence of the guide RNA (RSQ22337)), an in-frame sequence encoding the P2A self-cleaving peptide (“P2A”), an in-frame coding sequence as described above (“Cargo”), a stop codon and polyA signal sequence, and a 3′ homology arm approximately 500 bp in length (comprising a coding portion of exon 9 including a stop codon, the 3′ non-coding exonic region of exon 9, and a portion of the downstream intergenic sequence) (as shown in FIG. 3B). Four unique nucleofection events were conducted (corresponding to RNP and PLA1829, RNP and PLA1832, RNP and PLA1834, and RNP with no plasmid control) and cells were plated at clonal density. Colonies were propagated for analysis of TI using ddPCR.

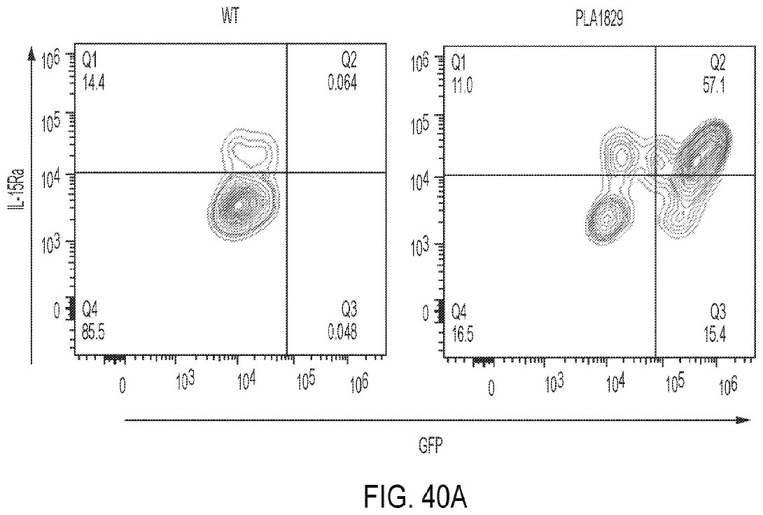

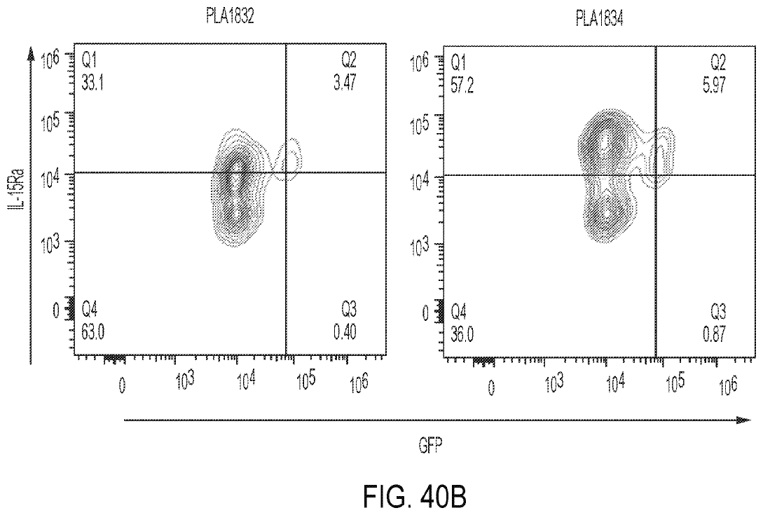

Following TI, transformed iPSCs (edited clones) with KI of PLA1829, PLA1832 or PLA1834 cargo sequences, or control WT parental cells transformed with RNP alone, were analyzed using flow cytometry seven days after transformation (see FIGS. 40A and 40B). The levels of GFP and IL-15Rα expression were measured in bulk edited iPSC populations. As shown in FIG. 40A, approximately 57% of cells transformed with PLA1829 expressed both IL-15Rα and GFP, while control cells had no GFP expression and approximately 14.4% IL-15Rα expression levels. As shown in FIG. 40B, approximately 33.1% of cells transformed with PLA1832, and approximately 57.2% of cells transformed with PLA1834 expressed IL-15Rα; neither of these cell populations displayed appreciable GFP levels, as expected as the respective donor templates did not comprise GFP. The expression of these cargo proteins can be used as a proxy for determining successful transformation, editing, and/or integration.

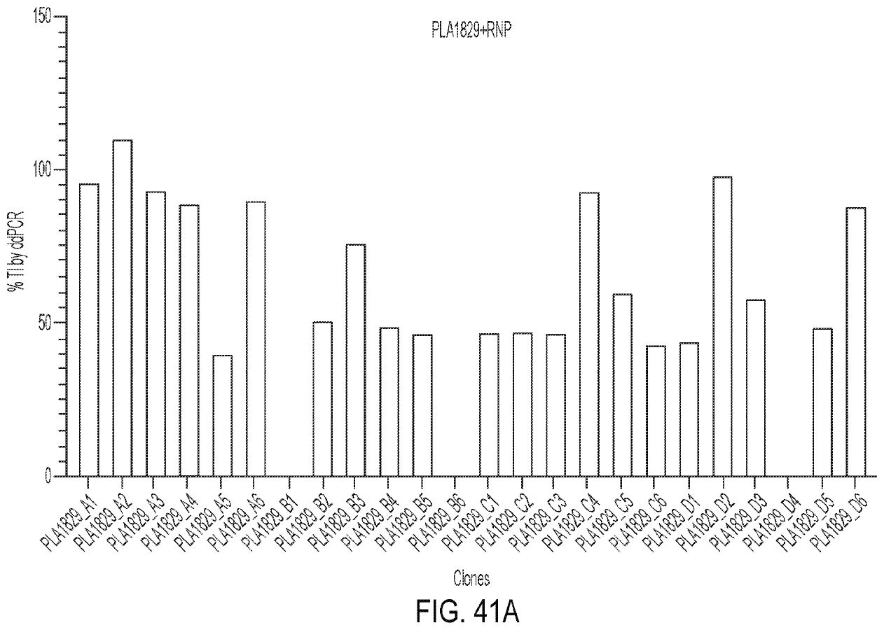

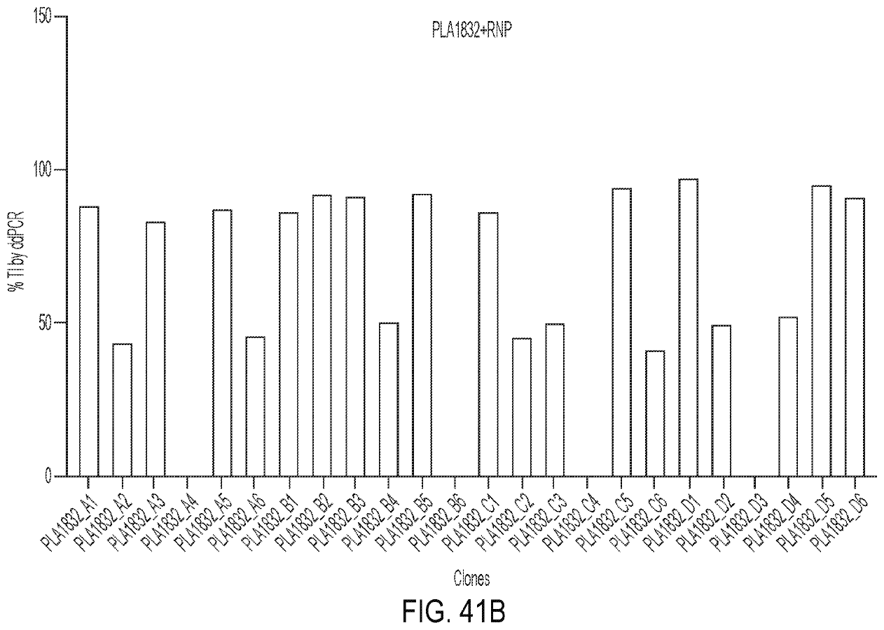

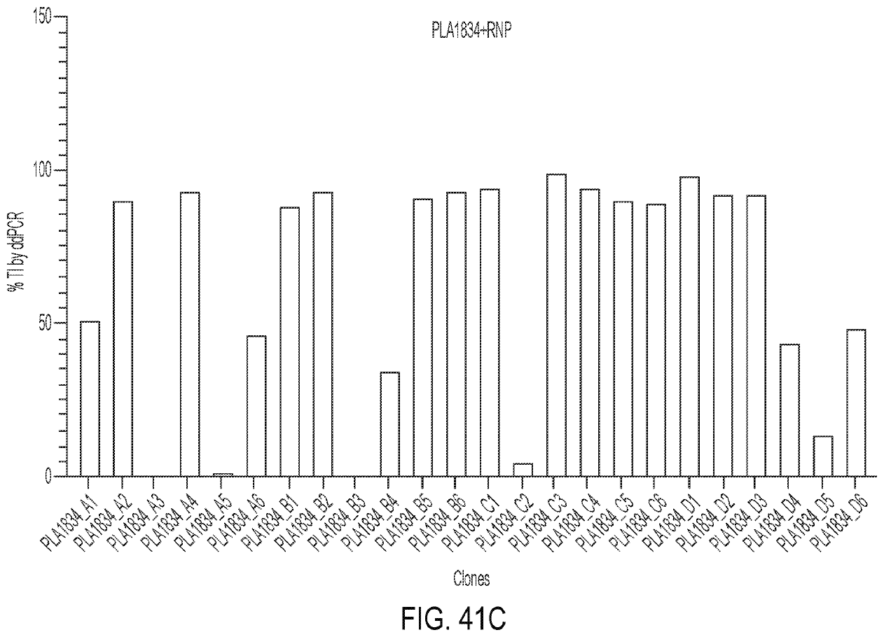

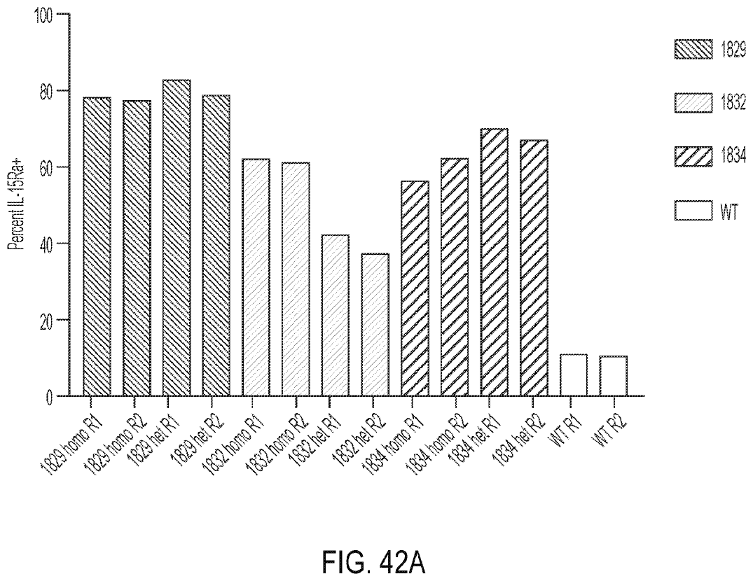

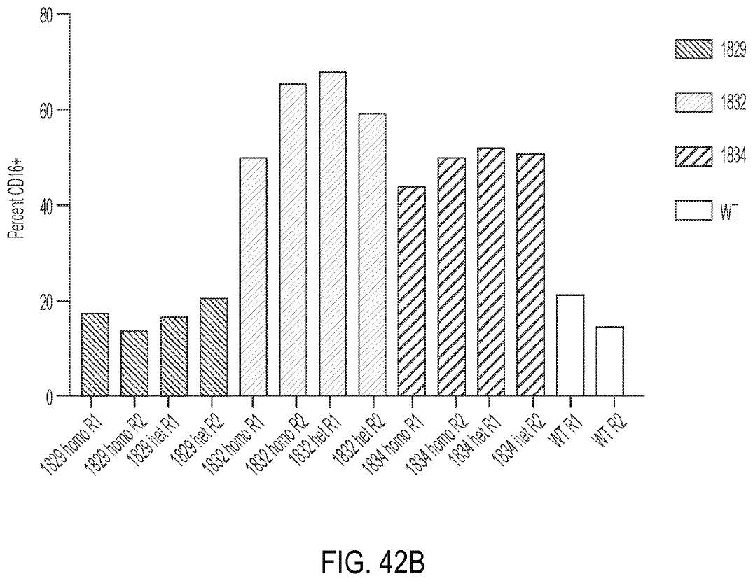

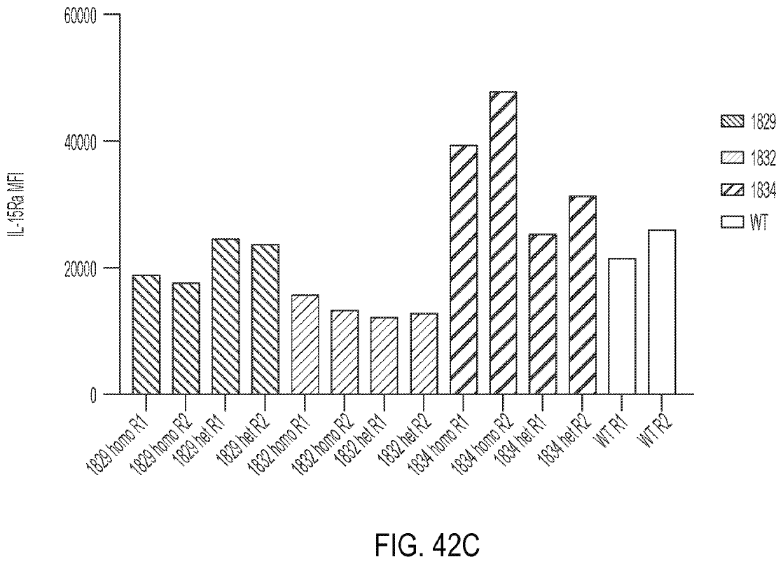

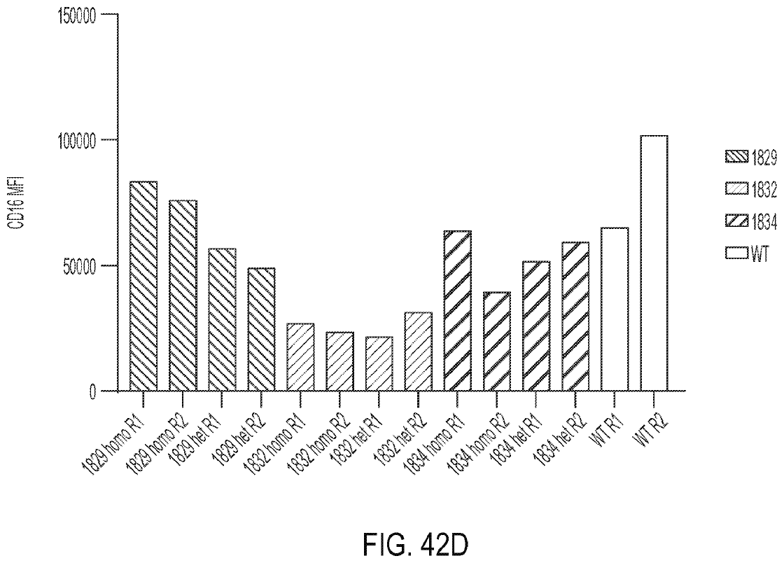

FIG. 41A-41C depicts the genotypes for 24 of the colonies transformed with PLA1829, PLA1832, or PLA1834 (comprising donor template SEQ ID NOs: 208, 209, and 212) respectively and compared to wild-type cells. Measured with ddPCR, cells with ˜85-100% TI are categorized as homozygous, 40-60% are categorized as heterozygous, while those with very low or no signal are categorized as wild type. The colonies were propagated after transformation, and cell populations were then differentiated to iNK cells using a spin embryoid method as known in the art. Shown in FIG. 42A-42D are exemplary flow cytometry results measuring the percentage of cells expressing IL-15Rα and/or CD16, and the median fluorescence intensity (MFI) of IL-15Rα and/or CD16 at day 32 of the iNK differentiation process. As shown in FIG. 42A, transformation with PLA1829, PLA1832, or PLA1834 enabled surface expression of IL-15Rα in heterozygous or homozygous colonies at significantly higher proportions than iNKs differentiated from control WT parental cells. As shown in FIG. 42B, transformation with PLA1832 or PLA1834 enabled surface expression of CD16 in heterozygous or homozygous colonies at significantly higher proportions than iNKs differentiated from control WT parental cells, as cells transformed with the PLA1829 cargo sequence do not comprise a CD16 cargo sequence. As shown in FIG. 42C, transformation with PLA1834 enabled higher MFI of IL-15Rα in heterozygous or homozygous colonies when compared to iNKs differentiated from control WT parental cells, or cells transformed with PLA1829 or PLA1832. As shown in FIG. 42D, transformation with PLA1832 or PLA1834 enabled surface expression of CD16 in heterozygous or homozygous colonies. These data show that the methods described herein can be used to knock-in a multicistronic cargo containing numerous genes of interest into an essential gene such as GAPDH, leading to expression of the genes of interest in the edited cells. These data also clearly demonstrate the constitutive nature of cargo expression from the GAPDH locus.

### Example 18—Computation Screening of AsCpf1 Guide RNAs Suitable for Selection by Essential-Gene Knock-In

The present example describes a method for computationally screening for AsCpf1 (AsCas12a; e.g., as represented by SEQ ID NO: 62) guide RNAs (gRNAs) suitable for methods described herein that target a number of essential housekeeping genes. The results of this screening are summarized in Table 17, these gRNAs facilitate Cas12a cleavage within the last 500 bp of the DNA coding sequences for the listed essential genes.

The essential genes in Table 17 selected for this analysis were identified in a pool of essential genes made by combining the essential genes described in Eisenberg et al., (see e.g., Eisenberg and Levanon, Human housekeeping genes, revisited. Trends Genetics, 2014) and the genes described in Yilmaz et al., (see e.g., Yilmaz et al., Defining essential genes for human pluripotent stem cells by CRISPR-Cas9 screening in haploid cells. Nature Cell Biology, 2018). In brief, essential genes described in Yilmaz et al., with CRISPR Scores less than 0, and FDR of <0.05 were combined with essential genes described in Eisenberg & Levanon to create a list of 4,582 genes in total. These genes were then sorted by their average expression level (mean normalized expression across different tissues, see e.g., RNA consensus tissue gene expression data provided by https://www.proteinatlas.org/download/rna_tissue_consensus.tsv.zip), and the 100 genes with the highest average expression levels across tissues were selected for the analysis. GAPDH was present within this group of genes. TBP, E2F4, G6PD and KIF11 were added to this group, making 104 genes in total, for further analysis.

Potential gRNA target sequences for each of the genes of interest were generated by searching for nuclease specific PAMs with suitable protospacers mapped to a representative coding region (mRNA-201). Transcripts with its name followed by “−201” were selected as the representative for each gene (e.g., GAPDH-201). Gene information (i.e., coding region) was obtained from GENCODE v.37 gene annotation GTF file. Potential gRNAs were first searched within the genomic regions of target genes in the human reference genome (hg38), and those identified gRNAs with their cut sites within 500 bp of the representative coding region's stop site were selected for further analysis. The candidate gRNAs were then aligned to the human reference genome (e.g., hg38) with BWA Aln (maximum mismatch tolerance −n 2). Guides with potential off target binding sites (i.e., aligning to multiple genomic regions; mapping quality MAPQ <30) were filtered out. The resultant gRNAs target highly and/or broadly expressed essential genes within 500 coding base pairs of a representative stop-codon and have no identical off-target binding sites annotated in the human genome. Thus, they are excellent candidate gRNAs for the selection methods described herein.

TABLE 17
AsCas12 guide RNAs

| SEQ | | Target |
|---|---|---|
| ID NO | Gene | Domain Sequence (DNA) |
| 225 | EIF4G2 | AGGCTTTGGCTGGTTCTTTAG |
| 226 | EIF4G2 | GCTGGTTCTTTAGTCAGCTTC |
| 227 | EIF4G2 | GTCAGCTTCTTCCTCTGATTC |
| 228 | EIF4G2 | TAACCAGGTTAGCCACTGATT |
| 229 | EIF4G2 | ACAAAAGACTTACCTGGAACA |
| 230 | EIF4G2 | CCGGAAACTCTTGGGTTATAT |
| 231 | EIF4G2 | CAAGCCAAGAAAGCTTCTTCT |
| 232 | EIF4G2 | CATGTCATAGAAGTGCACAAA |
| 233 | EIF4G2 | GGAAGTTGCTGTTATAGCAGT |
| 234 | EIF4G2 | TGCATTACTGGCTTGAAAGAT |
| 235 | EIF4G2 | CTGCTCTAACTGTTCTTTGGA |
| 236 | EIF4G2 | GAAGGAGCAGAGGATGAATCT |
| 237 | EIF4G2 | ATCGCTGGGGGGGTTTACTTC |
| 238 | EIF4G2 | CTTCACTAGAAATGTACTGTA |
| 239 | EIF4G2 | TCTACATGAAGTTTGGGAGAG |
| 240 | EIF4G2 | GGAGAGATGTTATCTTTAATC |
| 241 | EIF4G2 | TATATGGTTTGAGGGGATGGA |
| 242 | EIF4G2 | AGGGGATGGATCCAACTTTAT |
| 243 | EIF4G2 | TAGGTGAATCAGTGGCTAACC |
| 244 | EIF4G2 | CAAATCTTAATTTATAGGTGA |
| 245 | EIF4G2 | ATTTACAAATCTTAATTTATA |
| 246 | EIF4G2 | CGGGAAAAGGCAAGGCTTTGT |
| 247 | EIF4G2 | TTGGCTTGGAAAGAAGATATA |
| 248 | EIF4G2 | TGCACTTCTATGACATGGAAA |
| 249 | EIF4G2 | AGGCATGTTACTTCGCTTTTT |
| 250 | EIF4G2 | TTCATGATCACGTTGATCTAC |
| 251 | EIF4G2 | AAGCCAGTAATGCAGAAATTT |
| 252 | EIF4G2 | TAGTGAAGTAAACCCCCCCAG |
| 253 | EIF4G2 | TGTCCAGCTTCTTAGAGTACA |
| 254 | EIF4G2 | TGAACATCTTAATGACTAGGT |
| 255 | SKP1 | AAGACCTTACCTTTTTTAATA |
| 256 | SKP1 | CAATGAACTTACCTTCCAACA |
| 257 | SKP1 | AGCAGGGCAGAATAAAAACCA |
| 258 | SKP1 | TTCATAATTTCAGCAGGGCAG |
| 259 | SKP1 | CTTTGTTCATAATTTCAGCAG |
| 260 | SKP1 | CAGGCTGCAAACTACTTAGAC |
| 261 | SKP1 | TTGTTGTAGGTCATTCAGTGG |
| 262 | SKP1 | TTAGATTTGGGAATGGATGAT |
| 263 | SKP1 | TTCTGGTTTTCTTAGATTTGG |
| 264 | SKP1 | GATGCCTTCAATTAAGTTGCA |
| 265 | SKP1 | ATGTCCTTTTTTTTTAGATGC |
| 266 | RPS3 | AAGCTTTATGCTGAAAAGGTG |
| 267 | RPS3 | AAGGGCCTGCTATGGTGTGCT |
| 268 | RPS3 | AAGGAAGCAAGGGATATCCTG |
| 269 | RPS3 | AGCATAAAGCTTTAAAGGAAG |
| 270 | RPS3 | CCAGACACCACAACCTCGCAG |
| 271 | RPS3 | CCAAGCACTCTCAGCTGCTCA |
| 272 | RPS19 | TTCTTCCATCTTTTCCCACAG |
| 273 | RPS19 | CCACAGGTGGCAGCTGCCAAC |
| 274 | RPS19 | TCTGACGTCCCCCATAGATCT |
| 275 | HMGB1 | AGCCCTCTTACCTTCCACCTC |
| 276 | HMGB1 | TGTTCATTTATTGAAGTTCTA |
| 277 | HMGB1 | GTTCGGCCTTCTTCCTCTTCT |
| 278 | HMGB1 | TAGACCATGTCTGCTAAAGAG |
| 279 | HMGB1 | GAAAAATAACTAAACATGGGC |
| 280 | RPL7 | CCCCAAATAGAACCTACCAAG |
| 281 | RPL7 | ACTTCAGGTACCCCAATCTGA |
| 282 | RPL7 | CTTTTTCACTTCAGGTACCCC |
| 283 | RPL7 | TGTTTGCTTTTTCACTTCAGG |
| 284 | RPL7 | ACCACAGTATCAATGGAGTGA |
| 285 | RPL7 | TGGTCCGTTTTCACCACAGTA |
| 286 | RPLP0 | AGGTCAAGGCCTTCTTGGCTG |
| 287 | RPLP0 | ACCACTTCCCCCCTCCTTTCA |
| 288 | G6PD | CTCACCTGCCATAAATATAGG |
| 289 | G6PD | CAGTATGAGGGCACCTACAAG |
| 290 | G6PD | ACCCCACTGCTGCACCAGATT |
| 291 | G6PD | CGCCACGTAGGGGTGCCCTTC |
| 292 | RPL4 | GCTTGTAGTGCCGCTGCTGCA |
| 293 | RPL4 | CCGTGGTGCTCGAAGGGCTCT |
| 294 | RPL4 | TTGCAGCACAAGCTCCGGGTG |
| 295 | RPL4 | TGCCTAATTTGTTGCAGCACA |
| 296 | RPL4 | TAGCAAGAAGATCCATCGCAG |
| 297 | RPL4 | AGTCTTCCCATGCACAAGATG |
| 298 | RPL4 | CCTTTCAGTCTTCCCATGCAC |
| 299 | EEF1G | TCCCCAGCTGAGTCCAGATTG |
| 300 | EEF1G | TTCCTCTTAGTACCTTTGTGT |
| 301 | RPL31 | GATGGCTCCCGCAAAGAAGGG |
| 302 | RPL31 | AATCGTAGGGGCTTCAAGAAG |
| 303 | RPL31 | TTAGGAATGTGCCATACCGAA |
| 304 | RPL31 | CAGATCTACAGACAGTCAATG |
| 305 | RPL31 | GCACCTTATTCCTTTGGCCCA |
| 306 | RPL31 | TGGGATGGAGAACTTACTTTT |
| 307 | RPL31 | ATCTGACGATCAGCGATTAGT |
| 308 | ITM2B | ACTGTCTTTTTCATATTTTAG |
| 309 | ITM2B | ATATTTTAGGACCCAGATGAT |
| 310 | ITM2B | GGACCCAGATGATGTGGTACC |
| 311 | ITM2B | GACTAGCATTTATGCTTGCAG |
| 312 | ITM2B | TGCTTGCAGGTGTTATTCTAG |
| 313 | ITM2B | TGAATGTAGGCTGGAACCTAT |
| 314 | ITM2B | CCTCAGTCCTATCTGATTCAT |
| 315 | ITM2B | TTTATTTATCGACTGTGTCAT |
| 316 | ITM2B | TTTATCGACTGTGTCATGACA |
| 317 | ITM2B | TCGACTGTGTCATGACAAGGA |
| 318 | ITM2B | CCTCTCCAACAGGTATTCAGA |
| 319 | ITM2B | GCAATTCGGCATTTTGAAAAC |
| 320 | ITM2B | AAAACAAATTTGCCGTGGAAA |
| 321 | ITM2B | CCGTGGAAACTTTAATTTGTT |
| 322 | ITM2B | GCCAACTGGTACCACATCATC |
| 323 | ITM2B | TACAAGTATGCTCCTCCTAGA |
| 324 | ITM2B | CACTTACTTGAAGTGCAAAAT |
| 325 | ITM2B | AATGCGATCAGTAATAACCAT |
| 326 | ITM2B | CTTGTCATGACACAGTCGATA |
| 327 | ITM2B | TAAGTTTCCTTGTCATGACAC |
| 328 | ITM2B | TCTGCGTTGCAGTTTGTAAGT |
| 329 | ITM2B | ATAGTTTCTCTGCGTTGCAGT |
| 330 | ITM2B | AAAAGTATTACCTTTAATAGT |
| 331 | ITM2B | ATATTTAAAAAGTATTACCTT |
| 332 | ITM2B | AAAATGCCGAATTGCGAAACA |
| 333 | ITM2B | TTTTCAAAATGCCGAATTGCG |
| 334 | ITM2B | CACGGCAAATTTGTTTTCAAA |
| 335 | ITM2B | TTGACTGTTCAAGAACAAATT |
| 336 | RPL23A | CTTTTCTCCCAGCTCCTGCCC |
| 337 | RPL23A | TCCCAGCTCCTGCCCCTCCTA |
| 338 | RPL23A | CCTCTCCCAGGCTTGACCACT |
| 339 | RPL23A | TTTTTCAGATTGGGATCATCT |
| 340 | RPL23A | TAGGAAGGAAACTTACTTTGT |
| 341 | RPL27A | GTCTGGGCTGCCAACATGGTA |
| 342 | RPL27A | TATTCCTGCAGGCAAGCACCG |
| 343 | RPL27A | TCTGTTCTTCTAGGGCTACTA |
| 344 | PCBP2 | CCCTCTGACTCTCTCCCAGTC |
| 345 | PCBP2 | CTCCTTTTGTAGGCCTATACC |
| 346 | PCBP2 | TAGGCCTATACCATTCAAGGA |
| 347 | PCBP2 | CTCCTTGCAGTTGACCAAGCT |
| 348 | PCBP2 | ACTTGTATCTTAACAGGCATT |
| 349 | PCBP2 | GCAGGTTTGGATGCATCTGCT |
| 350 | PCBP2 | TTTCTCCCTTAAGTTGATTGG |
| 351 | PCBP2 | TCCCTTAAGTTGATTGGCTGC |
| 352 | PCBP2 | TGTGTTACAGGCTTTCCTCGG |
| 353 | PCBP2 | AGCATGAGCCTGAGGGCTTAC |
| 354 | PCBP2 | TTACCTGACCACCTGCAAAGA |
| 355 | PCBP2 | ATCATTAGCCCAATAGCCTTT |
| 356 | HSPA8 | TCTTCCTCAGACTGCTGAGAA |
| 357 | HSPA8 | CTAGGCCGTTTGAGCAAGGAA |
| 358 | HSPA8 | TTTCCTAGGCCGTTTGAGCAA |
| 359 | HNRNPK | ATCAGCACTGAAACCAACCTG |
| 360 | HNRNPK | AGTTGGCTGGATCTATTATTG |
| 361 | HNRNPK | AAAAATCTTTTCAGTTGGCTG |
| 362 | HNRNPK | AATCAGATTATTCCTATGCAG |
| 363 | HNRNPK | TGTTTTTAGGGTGGCTCCGGA |
| 364 | HNRNPK | TTTCTGTTTTTAGGGTGGCTC |
| 365 | HNRNPK | TCTCTAACAGGTTGGTTTCAG |
| 366 | RPL5 | TCTCTTACTATAGATTGCTTA |
| 367 | RPL5 | CATTGGTTTCTTGAATAGCTT |
| 368 | RPL5 | TTGAATAGCTTCTCAATAGGT |
| 369 | UBL5 | TGTAGCTCCAGCTAGGATGAT |
| 370 | UBL5 | CCTTAACTGCTCTGCGCCCAG |
| 371 | UBL5 | TTAGGTACACGATTTTTAAGG |
| 372 | UBL5 | CTTCAGATGAAATCCACGATG |
| 373 | CST3 | GACAAGGTCATTGTGCCCTGC |
| 374 | CST3 | AGATGTGGCTGGTCATGGAAG |
| 375 | CST3 | TTGTACTCGCCGACGGCAAAG |
| 376 | CST3 | CAGATCTACGCTGTGCCTTGG |
| 377 | CST3 | ACAGAAAGCATTCTGCTCTTT |
| 378 | CST3 | CTTTCACAGAAAGCATTCTGC |
| 379 | CST3 | ACATGTGTAGATCGTAGCTGG |
| 380 | CST3 | CCGTCGGCGAGTACAACAAAG |
| 381 | RPS29 | TCACCAAGAGCGAGAACCCTG |
| 382 | RPS29 | TTACAGTCGTGTCTGTTCAAA |
| 383 | RPS10 | TACTGTACATGCTTCCTTTTT |
| 384 | RPS10 | GAAATGACATTATCTGAGAGC |
| 385 | RPS10 | CTCACGTGGCACAGCACTCCG |
| 386 | RPS10 | TGTGGGAACCATACCTTTAGG |
| 387 | RPS10 | TAAAAAGGAAGCATGTACAGT |
| 388 | RPS10 | TCCTATGGCAGGTCCTCATAG |
| 389 | RPS10 | TAGCTGGTGCCGACAAGAAAG |
| 390 | RPS10 | ACTTTCTAGCTGGTGCCGACA |
| 391 | RPS10 | CATAGGTCTGGAGGGTGAGCG |
| 392 | RPS10 | ATTTACATAGGTCTGGAGGGT |
| 393 | RPS10 | TGCCTTACAGTCTCTCAAGTC |
| 394 | RPL6 | TTACGAGTCACAAGTAATAAG |
| 395 | RPL6 | GAAATATGAGATTACGGAGCA |
| 396 | RPL6 | TTTAGAAATATGAGATTACGG |
| 397 | RPL6 | TCTTTATTTAGAAATATGAGA |
| 398 | RPL6 | ATTTTCTCTTTATTTAGAAAT |
| 399 | RPL6 | CCCCTTAGGACCTCTGGTCCT |
| 400 | RPL6 | ACTTACAGAGGGTGGTTTTCC |
| 401 | RPL6 | TTTTTAACTTACAGAGGGTGG |
| 402 | RPLP2 | TGTAGGTATTGGCAAGCTTGC |
| 403 | ARF1 | ACACTGGCTGCCCGGCAGGCC |
| 404 | RPL15 | TGTGTAGGTTACGTTATATAT |
| 405 | RPL15 | CTATTCTAGGAGCGAGCTGGA |
| 406 | RPL15 | CCTCTGCAACGGACTGAAGGC |
| 407 | FAU | CTGGCCGGTCACCTCGAAGGT |
| 408 | FAU | CCTGTAGGCTCATGTAGCCTC |
| 409 | FAU | CTCAGTCGCCAATATGCAGCT |
| 410 | FAU | TTTACTCAGTCGCCAATATGC |
| 411 | RPL36 | CCCCCTAGCGTCTGACCAAAC |
| 412 | RPL36 | CCCCGTACGAGCGGCGCGCCA |
| 413 | NACA | CTAGTATACCTCTTCCTCTTC |
| 414 | NACA | CTCACCTTGGCTTCCCCAAAA |
| 415 | NACA | AAATCTTACCTTCCGTGCCTT |
| 416 | NACA | TCTGTTACAGGAATTAACAAT |
| 417 | NACA | CCTCTCATCTCTCAGGTCGAT |
| 418 | NACA | TACCCTGTAGATCGAAGATTT |
| 419 | NACA | GGCTATGTCCAAACTGGGTCT |
| 420 | NACA | TCTTCTTTAGGCTATGTCCAA |
| 421 | NACA | TCTTCTTAGCTGGCGGCAGCA |
| 422 | PRDX1 | GACATCAGGCTTGATGGTATC |
| 423 | PRDX1 | CCATGCTAGATGACAGAAGTG |
| 424 | PRDX1 | TTAAATTCTTCTGCCCTATCA |
| 425 | PRDX1 | TCTTGCAGTGTGCCCAGCTGG |
| 426 | PRDX1 | TCATTGATGATAAGGGTATTC |
| 427 | PRDX1 | CCAGGGGCCTTTTTATCATTG |
| 428 | PRDX1 | ATCTCTTTTCCCAGGGGCCTT |
| 429 | PRDX1 | CTTTCATCTCTTTTCCCAGGG |
| 430 | PRDX1 | GTATCAGACCCGAAGCGCACC |
| 431 | PRDX1 | CCATAGGGTCAATACACCTAA |
| 432 | PRDX1 | CCTTTTGCCATAGGGTCAATA |
| 433 | PRDX1 | AGTGATAGGGCAGAAGAATTT |
| 434 | PRDX1 | CCCTCTTGACTTCACCTTTGT |
| 435 | PRDX1 | CCCCCAGGAAAATATGTTGTG |
| 436 | ALDOA | CCTTCTCGGTCACATACTGGC |
| 437 | NCL | GCCCAGTCCAAGGTAACTTTA |
| 438 | NCL | TTTCCATCAATTTCACCGTCT |
| 439 | NCL | CATCAATTTCACCGTCTTCCA |
| 440 | NCL | ACCGTCTTCCATGGCCTCCTT |
| 441 | NCL | GCATCCTCCTCACTGTTGAAG |
| 442 | NCL | GAGGACCCAGTTTCCCGGTCA |
| 443 | NCL | CCGGTCAGTAACTATCCTTGC |
| 444 | NCL | ATGTCTCTTCAGTGGTATCCT |
| 445 | NCL | ACAAACAGAGTTTTGGATGGC |
| 446 | NCL | GTGGCAGAGGCCGGGGAGGCT |
| 447 | NCL | GAGGACGAGGTGGTGGTAGAG |
| 448 | NCL | TAGACTTCAACAGTGAGGAGG |
| 449 | NCL | GTTTTGTAGACTTCAACAGTG |
| 450 | NCL | GTGTTCTAGGTTTGGTTTTGT |
| 451 | NCL | ATTTGGTGTTCTAGGTTTGGT |
| 452 | NCL | ACGGCTCCGTTCGGGCAAGGA |
| 453 | NCL | TCAAAGGCCTGTCTGAGGATA |
| 454 | NCL | CTTCCCAGAGCCATCCAAAAC |
| 455 | BTF3 | TAGATGAAAGAAACAATCATG |
| 456 | BTF3 | CTCTTCTCCCTGACTTTAGGG |
| 457 | BTF3 | GGGAACTGCTCGCAGAAAGAA |
| 458 | BTF3 | TTTTCTTAATAGGTGAATATG |
| 459 | BTF3 | TTAATAGGTGAATATGTTTAC |
| 460 | BTF3 | CATTTTCCTTTCATAGCTGTG |
| 461 | BTF3 | CTTTCATAGCTGTGGATGGAA |
| 462 | BTF3 | ATAGCTGTGGATGGAAAAGCA |
| 463 | BTF3 | TACTCTTTTCCTTTTCCTAGA |
| 464 | BTF3 | CTTTTCCTAGATCTTGTGGAG |
| 465 | BTF3 | CTAGATCTTGTGGAGAATTTT |
| 466 | BTF3 | ATACTTGCCTCTTCAATACCA |
| 467 | E2F4 | GGGGCTATCATTGTAGTGAGT |
| 468 | E2F4 | AGCCCATCAAGGCAGACCCCA |
| 469 | E2F4 | AGTTTTGGAACTCCCCAAAGA |
| 470 | E2F4 | GAACTCCCCAAAGAGCTGTCA |
| 471 | E2F4 | CCCCTCTGCTTCGTCTTTCTC |
| 472 | E2F4 | TCCACCCCCGGGAGACCACGA |
| 473 | E2F4 | ATGTGCCTGTTCTCAACCTCT |
| 474 | E2F4 | TGACAGCTCTTTGGGGAGTTC |
| 475 | KIF11 | ACTAAGCTTAATTGCTTTCTG |
| 476 | KIF11 | TGGAACAGGATCTGAAACTGG |
| 477 | KIF11 | TACCCATCAACACTGGTAAGA |
| 478 | KIF11 | TTCTTTTAGGATGTGGATGTA |
| 479 | KIF11 | GGATGTGGATGTAGAAGAGGC |
| 480 | KIF11 | CCGCCTTAAATCCACAGCATA |
| 481 | KIF11 | ATTAAGTTCTAGATTTTGTGC |
| 482 | KIF11 | TGGTTTCATTAAGTTCTAGAT |
| 483 | KIF11 | AGATCCTGTTCCAGAAAGCAA |
| 484 | KIF11 | AAGTACCTGTTGGGATATCCA |
| 485 | KIF11 | TCTTTTAAAGTACCTGTTGGG |
| 486 | KIF11 | AGCTGATCAAGGAGATGTTGA |
| 487 | KIF11 | CTTTTCAGGTGATCAAGGAGA |
| 488 | KIF11 | GCATCATTAACAGCTCAGGCT |
| 489 | KIF11 | TGAACAGTTTAGCATCATTAA |
| 490 | KIF11 | TTGTTTTCTGAACAGTTTAGC |
| 491 | KIF11 | CCGGAATTGTCTCTTCTTTGT |
| 492 | KIF11 | AATTTACCGGAATTGTCTCTT |
| 493 | KIF11 | TCTTTTCCATGTGATTTTTTA |
| 494 | KIF11 | TTTGTCTTTTCCATGTGATTT |
| 495 | KIF11 | GACCTCTCCAGTGTGTTAATG |
| 496 | KIF11 | TTCCACTTTAGACCTCTCCAG |
| 497 | KIF11 | TAACCAAGTGCTCTGTAGTTT |
| 498 | RPL13 | TCTTCTAGGTCTATAAGAAGG |
| 499 | RPL13 | AGTAAGTGTTCACTTACGTTC |
| 500 | PFDN5 | CCTTAATTCTTGCTTCTCAGA |
| 501 | PFDN5 | AGCTGAGCAATGGACGTGGAC |
| 502 | PTMA | AAGGACTTAAAGGAGAAGAAG |
| 503 | PTMA | TGTCGAGGAGAATGAGGAAAA |
| 504 | PTMA | ATTCTCTCCAGGTGAGGAAGA |
| 505 | PTMA | TCTGCTTAGGATGACGATGTC |
| 506 | RPL11 | GCATCCGGAGAAATGAAAAGA |
| 507 | RPL11 | TCCACAGGTGCGGGAGTATGA |
| 508 | RPL11 | AGCATCGCAGACAAGAAGCGC |
| 509 | RPL11 | AGTATGATGGGATCATCCTTC |
| 510 | RPL11 | CGGATGCGAAGTTCCCGCATG |
| 511 | RPL11 | TCCGGATGCCAAAGGATCTGA |
| 512 | RPL11 | ATTTCTCCGGATGCCAAAGGA |
| 513 | RPL11 | GACCCTTCTCCAAGATTTCTT |
| 514 | RPL11 | TTAACTCATACTCCCGCACCT |
| 515 | RPL11 | CCTTCTGCTGGAACCAGCGCA |
| 516 | COX7C | TCTTTTTTTCCAACAGAATTT |
| 517 | COX7C | CAACAGAATTTGCCATTTTGA |
| 518 | RPL8 | TTGAGGCCCTCAGCACTAGTT |
| 519 | RPL8 | CGGCCAGCAGGGGCATCTCTG |
| 520 | RPL8 | TGGGTTACTTACATTCATGGC |
| 521 | RPL8 | TCTGCCTGCAGCCTGTGGAGC |
| 522 | RPL10 | TTCTCCCTACCTAGCCCTGGA |
| 523 | RPL10 | CATTGCTCCTTAGATCCACAT |
| 524 | RPL32 | CCTCCCCAAAAGGAAGAGTTC |
| 525 | TBP | CTGCGGTAATCATGAGGATAA |
| 526 | TBP | AGTTCTGGGAAAATGGTGTGC |
| 527 | TBP | CTTTCCCTAGTGAAGAACAGT |
| 528 | TBP | CCTAGTGAAGAACAGTCCAGA |
| 529 | TBP | CAGCTAAGTTCTTGGACTTCA |
| 530 | TBP | CTATAAGGTTAGAAGGCCTTG |
| 531 | TBP | CAATTTTCCTTCTAGTTATGA |
| 532 | TBP | CTTCTAGTTATGAGCCAGAGT |
| 533 | TBP | CTGGTTTAATCTACAGAATGA |
| 534 | TBP | ATCTACAGAATGATCAAACCC |
| 535 | TBP | TTTCTGGAAAAGTTGTATTAA |
| 536 | TBP | TGGAAAAGTTGTATTAACAGG |
| 537 | TBP | GGTCAAGTTTACAACCAAGAT |
| 538 | TBP | GGGCACGAAGTGCAATGGTCT |
| 539 | TBP | CCAGAACTGAAAATCAGTGCC |
| 540 | TBP | TTACGGCTACCTCTTGGCTCC |
| 541 | TBP | TTGCTGCCAGTCTGGACTGTT |
| 542 | TBP | AGACTTAGCTACTAAATTGTT |
| 543 | TBP | ATCATTCTGTAGATTAAACGA |
| 544 | TBP | CAGAAACAAAAATAAGGAGAA |
| 545 | TBP | AAATGCTTCATAAATTTCTGC |
| 546 | CD63 | CTCAGCCAGCCCCCAATCTTC |
| 547 | CD63 | TCCCAATCTGTGTAGTTAGCA |
| 548 | CD63 | GGGTAATTCTCCATCTGCTGC |
| 549 | CD63 | GGAATTGTCTTTGCCTGCTGC |
| 550 | CD63 | CTTCTAGGTTTTGGGAATTGT |
| 551 | CD63 | TGCCTGCCACCTTCAGGGCTG |
| 552 | CD63 | AACGAGAAGGCGATCCATAAG |
| 553 | CD63 | AGTGCTGTGGGGCTGCTAACT |
| 554 | CD63 | TTCCCTCCCCCAGTTTAAGTG |
| 555 | CD63 | ATAACAACTTCCGGCAGCAGA |
| 556 | CD63 | TGTCTCTTATCATGTTGGTGG |
| 557 | CD63 | CCATCTTTCTGTCTCTTATCA |
| 558 | CD63 | CTCCTGCAGTTTGCCATCTTT |
| 559 | CD63 | TGGGCTGCTGCGGGGCCTGCA |
| 560 | RPS24 | TGTTTTCAGAACGACACCGTA |
| 561 | RPS24 | AGAACGACACCGTAACTATCC |
| 562 | RPS24 | GGTCATTGATGTCCTTCACCC |
| 563 | RPS24 | TCATTCAGCATGGCCTGTATG |
| 564 | RPS24 | CCTCTTCTTCTGGATTACAGA |
| 565 | RPS24 | TAGTGCGGATAGTTACGGTGT |
| 566 | RPS24 | CTTAATGAACTATACCTTTTT |
| 567 | RPS23 | GGGCTGTGCCCAAATGAGCTT |
| 568 | RPS23 | TTCCAGGAAAATGATGAAGTT |
| 569 | RPS23 | TACCCAATGACGGTTGCTTGA |
| 570 | RPS23 | AGAGGAGTTGAAGCCAAACAG |
| 571 | RPS23 | TATTTCAGAGGAGTTGAAGCC |
| 572 | RPS23 | GGCAAGTGTCGTGGACTTCGT |
| 573 | RPS23 | ATTTTTAGGCAAGTGTCGTGG |
| 574 | EEF2 | TCCAGGAAGTTGTCCAGGGCA |
| 575 | EEF2 | AGGCCCTTGCGCTTGCGGGTC |
| 576 | EEF2 | ACCACTGGCAGATCCTGCCCG |
| 577 | EEF2 | TGGTCAAGGCCTATCTGCCCG |
| 578 | EEF2 | AACAGGAAGCGGGGCCACGTG |
| 579 | EEF2 | CCTTCTGGCAGTGTCCAGAGC |
| 580 | EEF2 | TTTCCCTTCTGGCAGTGTCCA |
| 581 | CALR | CTTCTCCCTTCTGCAGGGTGA |
| 582 | CALR | GCGTGCTGGGCCTGGACCTCT |
| 583 | CALR | ACAACTTCCTCATCACCAACG |
| 584 | CALR | GCAACGAGACGTGGGGCGTAA |
| 585 | CALR | TGGGTGGATCCAAGTGCCCTT |
| 586 | CALR | CTCCAAGTCTCACCTGCCAGA |
| 587 | CALR | TTACGCCCCACGTCTCGTTGC |
| 588 | CALR | TCCTTCATTTGTTTCTCTGCT |
| 589 | CALR | TTGTCTTCTTCCTCCTCCTTA |
| 590 | CALR | TCCTCATCATCCTCCTTGTCC |
| 591 | RPL36AL | TATGCCCAGGGAAGGAGGCGC |
| 592 | SRP14 | AGGCTTATTCAAACCTCCTTA |
| 593 | SRP14 | AGGTGAGCTCCAAGGAAGTGA |
| 594 | SRP14 | CTTCTTTTTCAGGTGAGCTCC |
| 595 | SRP14 | CTTCAGATGACGGTCGAACCA |
| 596 | SRP14 | CAGAAGTGCCGGACGTCGGGC |
| 597 | SRP14 | CAGTTCCTGACGGAGCTGACC |
| 598 | GABARAP | TTTCGGATCTTCTCGCCCTCA |
| 599 | GABARAP | GGATCTTCTCGCCCTCAGAGC |
| 600 | GABARAP | TCTACATTGCCTAGAGTGACG |
| 601 | GABARAP | ATCCCAGGAACACCATGAAGA |
| 602 | GABARAP | TGCTTTCATCCCAGGAACACC |
| 603 | GABARAP | TCAACAATGTCATTCCACCCA |
| 604 | GABARAP | TTTGTCAACAATGTCATTCCA |
| 605 | GABARAP | CAGTTGGTCAGTTCTACTTCT |
| 606 | GABARAP | TTGCATCTTGTATCTTTTGCA |
| 607 | GABARAP | TCAGGTGATAGTAGAAAAGGC |
| 608 | GABARAP | ATCTCTTTATCAGGTGATAGT |
| 609 | RPSA | ATAATCTGCCACTCTTGGCAG |
| 610 | RPSA | TAACCCAGATTGAAAAAGAAG |
| 611 | RPSA | GTATTCTCTTAACAGAAGACT |
| 612 | RPSA | GAGAAGCTTACCTCTTCAGGA |
| 613 | SET | AATTATTTATTACAGTATTTT |
| 614 | SET | TTACAGTATTTTGATGAAAAT |
| 615 | SET | GGATTTGACGAAACGTTCGAG |
| 616 | SET | ACGAAACGTTCGAGTCAAACG |
| 617 | SET | AGGTTCCCGATATGGATGATG |
| 618 | SET | TTTCAGGAGGATGAAGGAGAA |
| 619 | SET | AGGAGGATGAAGGAGAAGATG |
| 620 | SET | TTTTACCTCTCCTTCCTCCCC |
| 621 | SET | GCCAAATTTTCTTTTACCTCT |
| 622 | GAPDH | CAGACCACAGTCCATGCCATC |
| 623 | GAPDH | ATCTTCTAGGTATGACAACGA |
| 624 | RPLP1 | TTTGTTGTAGGAGGATAAGAT |
| 625 | RPLP1 | TTGTAGGAGGATAAGATCAAT |
| 626 | RPLP1 | TAGCTGAGGAGAAGAAAGTGG |
| 627 | RPLP1 | CCACCATCACCTTACCTTTGC |
| 628 | RPLP1 | CTACCTGGAGCAGCAGCAGTG |
| 629 | CFL1 | CTCTTAAGGGGCGCAGACTCG |
| 630 | CFL1 | TAGGGATCAAGCATGAATTGC |
| 631 | CFL1 | TTCTTTATAGGGATCAAGCAT |
| 632 | CFL1 | TGTCCAGGGCCCCCGAGTCTG |
| 633 | RPS15 | CTCTTGGTCTCCCGCAGCCCG |
| 634 | TPT1 | CATTATTTATTTTAACCCACT |
| 635 | TPT1 | TTTTAACCCACTTCCTTGTAC |
| 636 | TPT1 | ACCCACTTCCTTGTACTTACA |
| 637 | TPT1 | CCTGGTAGTTTTTGAAATTAG |
| 638 | TPT1 | GAAATGGAAAAATGTGTAAGT |
| 639 | TPT1 | CTTCCCAAGTTCTTTATTGGT |
| 640 | TPT1 | TTTGCTTCCCAAGTTCTTTAT |
| 641 | TPT1 | GAATCAAAGGGAAACTTGAAG |
| 642 | TPT1 | TTAATGCAGATGGTCAGTAGG |
| 643 | RPL23 | CTACCTTTCATCTCGCCTTTA |
| 644 | RPL23 | TTGTTCACTATGACTCCTGCA |
| 645 | RPL23 | CTCACCCTTTTTTCTGAGCTC |
| 646 | RPL23 | ATGCAGGTTCTGCCATTACAG |
| 647 | RPL23 | TTTTTTTAATGCAGGTTCTGC |
| 648 | RPL23 | TTCTCTCAGTACATCCAGCAG |
| 649 | RPL34 | ACTTTCTAGGTCCCGAACCCC |
| 650 | RPL34 | TAGGTCCCGAACCCCTGGTAA |
| 651 | RPL34 | TTATGCAGGTTCGTGCTGTAA |
| 652 | RPL34 | GTATTTTCCTTTCTAGGATCA |
| 653 | RPL34 | CTTTCTAGGATCAAGCGTGCT |
| 654 | RPL34 | TAGGATCAAGCGTGCTTTCCT |
| 655 | RPL34 | AGAAATACTTACAGCCTAGTT |
| 656 | RPL34 | ACTTACCTGTCACGAACACAT |
| 657 | RPL34 | AGCATTTAACTTACCTGTCAC |
| 658 | COX4I1 | TCTTTCAGAATGTTGGCTACC |
| 659 | COX4I1 | AGAATGTTGGCTACCAGGGTA |
| 660 | COX4I1 | CACCTCTGTGTGTGTACGAGC |
| 661 | COX4I1 | TTCAATATGTTTTTCAGAAAG |
| 662 | COX4I1 | AGAAAGTGTTGTGAAGAGCGA |
| 663 | COX4I1 | GCTCCCAGCTTATATGGATCG |
| 664 | COX4I1 | CTGAGATGAACAGGGGCTCGA |
| 665 | COX4I1 | ACCGCGCTCGTTATCATGTGG |
| 666 | COX4I1 | ACAAAGAGTGGGTGGCCAAGC |
| 667 | COX4I1 | TCAAAGCTTTGCGGGAGGGGG |
| 668 | COX4I1 | GTAGTCCCACTTGGAGGCTAA |
| 669 | RPL27 | TCCTTGCTCTCTGCAGAAATG |
| 670 | RPL27 | GAACATTGATGATGGCACCTC |
| 671 | RPL27 | TCCCCAGGTACTCTGTGGATA |
| 672 | RPL27 | CCTTCTAGATACAAGACAGGC |
| 673 | RPL27 | CGTCCGGAGTAGCGTCCAGCC |
| 674 | RPL27 | TCTTTGATCTCTTGGCGATCT |
| 675 | RPL27 | ACAAAAGATTTTATCTTTGAT |
| 676 | EDF1 | GAGGCTTTGTGTTCATTTCGC |
| 677 | EDF1 | TGTTCATTTCGCCCTAGGCCC |
| 678 | EDF1 | GCCCTAGGCCCCTTCTCGATG |
| 679 | EDF1 | CAATGTCCTTTCCCCGGAGCT |
| 680 | EDF1 | CCAAGCACCTGGTTATTGGGT |
| 681 | EDF1 | TTGGAAGTCTCCACATCTTCT |
| 682 | EDF1 | GCCTGGGCGGCCGTAGGGCCC |
| 683 | EDF1 | AGGCCTCAAGCTCCGGGGAAA |
| 684 | EDF1 | GAAAATCAATGAGAAGCCACA |
| 685 | EDF1 | CCTCACACCGACTCCAGGGGC |
| 686 | EDF1 | TAGGCTATCTTAGCGGCACAG |
| 687 | EDF1 | TAATTTTCTAGGCTATCTTAG |
| 688 | TMEM59 | AAAGAAAAATGCTTAAATTTC |
| 689 | TMEM59 | AGAATGAGCAAGATTCACTTT |
| 690 | TMEM59 | TAGGTAGAGGCCCTGCTTCTT |
| 691 | TMEM59 | GATCTAACAACCACAAGAGAA |
| 692 | TMEM59 | GCTTTTGTTCATTCATAAACT |
| 693 | TMEM59 | TTCATTCATAAACTCCAAGTC |
| 694 | TMEM59 | CCTCAGAGGGAACATACTGCT |
| 695 | TMEM59 | TCCATCTTCAAGAAAATTCCT |
| 696 | TMEM59 | CTTAGAGATGATTCTCTCAAA |
| 697 | TMEM59 | TAGGCTCCTGCTCCAAATGTG |
| 698 | TMEM59 | CGTCATCGGCTTGAAGATAAA |
| 699 | TMEM59 | TGAATGAACAAAAGCTAAACA |
| 700 | TMEM59 | CAGAAGCTGAGTATCTATGGT |
| 701 | TMEM59 | TTTTGCAGAAGCTGAGTATCT |
| 702 | TMEM59 | TTGTGCAACTGTTGCTACAGC |
| 703 | TMEM59 | GATTTGTTGTGCAACTGTTGC |
| 704 | TMEM59 | ACTACAACTCTTGTCCTCTCG |
| 705 | TMEM59 | CAGTAACTCTGGGTGGATTTT |
| 706 | TMEM59 | TTGAAGATGGAGAAAGTGATG |
| 707 | TMEM59 | AGCAGATCTGCAAATGAGAAA |
| 708 | TMEM59 | AGAGAATCATCTCTAAGCAAA |
| 709 | TMEM59 | GAGCAGGAGCCTACAAATTTG |
| 710 | TMEM59 | GTCTAAGCCAGAAATCCAGTA |
| 711 | TMEM59 | ATTATTATTTTAGTCTAAGCC |
| 712 | TMEM59 | TCTTCAAGCCGATGACGGAAA |
| 713 | DYNLL1 | TCTTTTCCAGGAATTTGACAA |
| 714 | DYNLL1 | CAGGAATTTGACAAGAAGTAG |
| 715 | DYNLL1 | ATGTGTCACATAACTACCGAA |
| 716 | NME2 | TTTCTTAGGAACATCATTCAT |
| 717 | NME2 | TTAGGAACATCATTCATGGCA |
| 718 | TMBIM6 | GCTGATGGCAACACCTCATAG |
| 719 | TMBIM6 | TGTTTTCTAGGAGTTGGCCTG |
| 720 | TMBIM6 | TAGGAGTTGGCCTGGGCCCTG |
| 721 | TMBIM6 | TATTGCTGTCAACCCCAGGTA |
| 722 | TMBIM6 | TAACAGCATCCTTCCCACTGC |
| 723 | TMBIM6 | ATGGGCACGGCAATGATCTTT |
| 724 | TMBIM6 | CCTGCTTCACCCTCAGTGCAC |
| 725 | TMBIM6 | CTGTGTCTTATAGGTATCTTG |
| 726 | TMBIM6 | TCTTCCCTGGGGAATGTTTTC |
| 727 | TMBIM6 | GATCCATTTGGCTTTTCCAGG |
| 728 | TMBIM6 | TTAGGCAAACCTGTATGTGGG |
| 729 | TMBIM6 | ATACTCAACTCATTATTGAAA |
| 730 | TMBIM6 | AGGCACTGCATTGATCTCTTC |
| 731 | TMBIM6 | ATTACTGTCTTCAGAAAACTC |
| 732 | TMBIM6 | TCCATTTCTAGGATAAGAAGA |
| 733 | TMBIM6 | TAGGATAAGAAGAAAGAGAAG |
| 734 | TMBIM6 | ATGGCTATGAGGTGTTGCCAT |
| 735 | TMBIM6 | TGTTCAGTTTCATGGCTATGA |
| 736 | TMBIM6 | CCAGTTCACACTTACCTCCCA |
| 737 | TMBIM6 | AATAATGAGTTGAGTATCAAA |
| 738 | TMBIM6 | TGAAGACAGTAATGAAATCTA |
| 739 | TMBIM6 | ATTCATGGCCAGGATCATCAT |
| 740 | TMBIM6 | GGTTGTAGGCTAACTAACCTT |
| 741 | RPS7 | TTTAGGAAATTGAAGTTGGTG |
| 742 | RPS7 | GGAAATTGAAGTTGGTGGTGG |
| 743 | RPS7 | CCTTACAGAGGAGAATTCTGC |
| 744 | RPS7 | AACTATTCTTTTAGCCGTACT |
| 745 | RPS7 | GCCGTACTCTGACAGCTGTGC |
| 746 | RPS7 | TTTTCTTGTAGGTTGAAACTT |
| 747 | RPS7 | TTGTAGGTTGAAACTTTTTCT |
| 748 | RPS7 | TGAAACTAGTAAAATACTCAC |
| 749 | ACTB | CTTCCCAGGGCGTGATGGTGG |
| 750 | NPM1 | ATTTGTAGTGATGATGATGAT |
| 751 | NPM1 | TAATTGCAGTCTATACGAGAT |
| 752 | NPM1 | GAAATTCATTTCTTTTTCAGG |
| 753 | NPM1 | TTTTTCAGGGACAAGAATCCT |
| 754 | NPM1 | AGGGACAAGAATCCTTCAAGA |
| 755 | NPM1 | TCTTAATAGGGTGGTTCTCTT |
| 756 | NPM1 | CAGGCTATTCAAGATCTCTGG |
| 757 | NPM1 | TAAAATCATACTTAGTCTTCA |
| 758 | NPM1 | CTCACTTTTTCTATACTTGCT |
| 759 | RPS6 | TTTTTCTTGGTACGCTGCTTC |
| 760 | RPS6 | GGGCCCAGGCGGCGAGGCACT |
| 761 | RPS6 | GGAGGCTAAGGAGAAGCGCCA |
| 762 | RPS6 | TTTAGGAGGCTAAGGAGAAGC |
| 763 | RPS6 | TTTTGTTTAGGAGGCTAAGGA |
| 764 | RPS6 | GGTAAGAAACCTAGGACCAAA |
| 765 | RPS6 | AATTTTTAGGTAAGAAACCTA |
| 766 | RPS6 | TTCTAAGGAGAGAAGGATATT |
| 767 | RPL12 | CTTAAAGGAACCATTAAAGAG |
| 768 | RPL12 | TTTACTTAAAGGAACCATTAA |
| 769 | RPL12 | CTCTTCTGCAGTTAAACACAG |
| 770 | RPL12 | CTGTTTCCTCTTCTGCAGTTA |
| 771 | RPL12 | TAGTCTCCAAAAAAAGTTGGT |
| 772 | RPL12 | TTTCTAGTCTCCAAAAAAAGT |
| 773 | RPL12 | CCCCAGTATACCTGAGGTGCA |
| 774 | CAPNS1 | AACCTGTTACCCACAGACCCT |
| 775 | CAPNS1 | GCATTGACACATGTCGCAGCA |
| 776 | CAPNS1 | AGGAATTCAAGTACTTGTGGA |
| 777 | CAPNS1 | CAGTAGTGAACTCCCAGGTGC |
| 778 | CAPNS1 | ATGTTGTTCCACAAGTACTTG |
| 779 | CAPNS1 | TACACACCTGCCACCTTTTGA |
| 780 | CAPNS1 | AGAGGTTTCTACACACCTGCC |
| 781 | CAPNS1 | ATCTGAGTAGCGTCGGATGAT |
| 782 | CAPNS1 | TCAAGAGATTTGAAGGCACCT |
| 783 | CAPNS1 | TCCAGTGCCATCTTTGTCAAG |
| 784 | RPL3 | CAGGGTGGCTTTGTCCACTAT |
| 785 | RPS13 | TTTATTAGCTTACCTTTCTGT |
| 786 | RPS13 | TTAGCTTACCTTTCTGTTCCT |
| 787 | RPS13 | AGTGAATCATCTACAGCCTCT |
| 788 | RPS13 | TTTTTCAGTGAATCATCTACA |
| 789 | RPS13 | CCCTTTTTTCTTTTTCAGTGA |
| 790 | RPS13 | AGGTGTAATCCTGAGAGATTC |
| 791 | RPS13 | TATTCCATAACAGTGGTTGAA |
| 792 | RPS21 | TCCACAGCTCCGCTAGCAATC |
| 793 | RPS21 | TGACCCTTCTTCTCTTTCTAG |
| 794 | RPS21 | TAGGTTGACAAGGTCACAGGC |
| 795 | RPS21 | TTAAGGGTGAGTCAGATGATT |
| 796 | RPS21 | CCCTGGTTCTAGGAACTTTTG |
| 797 | RPS21 | AGACGATGCCATCGGCCTTGG |
| 798 | SERF2 | ATTTTCTTTCCTTAGGCGGTA |
| 799 | SERF2 | TTTCCTTAGGCGGTAACCAGC |
| 800 | SERF2 | CTTAGGCGGTAACCAGCGTGA |
| 801 | SERF2 | TGCTGCCGCCCGCAAGCAGAG |
| 802 | SERF2 | ATATTCTTCTGGCGGGCGAGC |
| 803 | SERF2 | CCTTAACCGAGTCGCTCTGCT |
| 804 | SERF2 | CCTCCCCTCCCTGGGGCTACC |
| 805 | RPL7A | TTTCCCCTCCTGCCTTTTAGG |
| 806 | RPL7A | CCCTCCTGCCTTTTAGGGAAG |
| 807 | RPL7A | GGGAAGACAAAGGCGCTTTGG |
| 808 | RPL7A | TCTTTTCAGATCCGCCGTCAC |
| 809 | RPL7A | AGATCCGCCGTCACTGGGGTG |
| 810 | RPL7A | GGGCCAGGCTGTGTACTTACG |
| 811 | RPL7A | GTGTAAAGCTGCCTCTTACCT |
| 812 | HNRNPA2B1 | TAAATTACCTCCACCATATGG |
| 813 | HNRNPA2B1 | CACTCTTCATTGGACCGTAGT |
| 814 | HNRNPA2B1 | CAAAATCATTGTAATTTCCAC |
| 815 | HNRNPA2B1 | TTACCTCCTCCATAGTTGTCA |
| 816 | HNRNPA2B1 | CACCGCCACCACGTGAATCCC |
| 817 | HNRNPA2B1 | GTGGTAGCAGGAACATGGGGG |
| 818 | HNRNPA2B1 | GAAATTATAACCAGCAACCTT |
| 819 | HNRNPA2B1 | ATAGGAAATTATGGAAGTGGA |
| 820 | HNRNPA2B1 | GAGGTAGCCCCGGTTATGGAG |
| 821 | HNRNPA2B1 | TAATAGGTGGCAATTTTGGAG |
| 822 | HNRNPA2B1 | GGGATGGCTATAATGGGTATG |
| 823 | HNRNPA2B1 | GCCCCTAACAGATGGATATGG |
| 824 | HNRNPA2B1 | GGACCAGGACCAGGAAGTAAC |
| 825 | HNRNPA2B1 | GGGATTCACGTGGTGGCGGTG |
| 826 | HNRNPA2B1 | GCTTTGGGGATTCACGTGGTG |
| 827 | HNRNPA2B1 | TTGTAGGCAACTTTGGCTTTG |
| 828 | HNRNPA2B1 | TCTAGACAAGAAATGCAGGAA |
| 829 | RPL13A | TCTAACAGAAAAAGCGGATGG |
| 830 | RPL13A | GCATAGCTCACCTTGTCGTAG |
| 831 | ENO1 | AGCAGGAGGCAGTTGCAGGAC |
| 832 | ENO1 | TCCTTCCCAAGAATTGAAGAG |
| 833 | ENO1 | CCTTTCTCCTTCCCAAGAATT |
| 834 | ENO1 | TCCTAGATCAAGACTGGTGCC |
| 835 | ENO1 | TTTTCTCCTAGATCAAGACTG |
| 836 | ENO1 | CTTAGTGGTGTCTATCGAAGA |
| 837 | PPIA | CTATATGTTGACAGGGTGGTG |
| 838 | PPIA | AAGGTTGGATGGCAAGCATGT |
| 839 | CD81 | CCTGTGAGGTGGCCGCCGGCA |
| 840 | CD81 | ACCACCTCAGTGCTCAAGAAC |
| 841 | CD81 | TGTCCCTCGGGCAGCAACATC |
| 842 | RPL35 | TTGACAATGCGCCCCTCAGGC |
| 843 | RPL35 | TAGCCGAGTCGTCCGGAAATC |
| 844 | DAD1 | TTCTGTGGGTTGATCTGTATT |
| 845 | DAD1 | CCAGCACCATCCTGCACCTTG |
| 846 | DAD1 | TCTTTGCCAGCACCATCCTGC |
| 847 | DAD1 | CTGATTTTCTCTTTGCCAGCA |
| 848 | DAD1 | CAAGGCATCTCCCCAGAGCGA |
| 849 | DAD1 | CCTGAGAATACAGATCAACCC |
| 850 | DAD1 | CTTCTTGTGCAGTTTGCCTGA |
| 851 | DAD1 | TGTTTTGCTTCTTGTGCAGTT |
| 852 | DAD1 | TCTCGGGCTTCATCTCTTGTG |
| 853 | DAD1 | GCGGTTCTTAGAAGAGTACTT |
| 854 | UBA52 | TGAAGACCCTCACTGGCAAAA |
| 855 | UBA52 | CCAGTGAGGGTCTTCACAAAG |
| 856 | UBA52 | TGGGCAAGCTGGCGGAGAGAA |
| 857 | UBA52 | ACCTTCTTCTTGGGACGCAGG |
| 858 | RPL30 | TAGGTGAAAAGGTTTACTTTT |
| 859 | RPL30 | TGATTTAAAAAGCATACCTGG |
| 860 | RPL30 | AAAAGCATACCTGGATCAATG |
| 861 | RPL30 | GGTGACTCTGACATCATTAGA |
| 862 | RPL30 | TTTTTTAGGTGACTCTGACAT |
| 863 | RPL30 | TTTTTATTTTTTAGGTGACTC |
| 864 | RPL30 | GTTCCCAAAGGAAATCTGAAA |
| 865 | RPL30 | CCCATTTTGGTTCCCAAAGGA |
| 866 | RPL30 | TAGAAAAAGTCGCTGGAGTCG |
| 867 | RPL30 | CTTTGTAGAAAAAGTCGCTGG |
| 868 | RPL30 | ATGTTTGCTTTGTAGAAAAAG |
| 869 | RNASEK | CGCCTGCCGCCCCCGGATGGG |
| 870 | RNASEK | TCCCACCGCTTTCCGAGCCCG |
| 871 | RNASEK | CGAGCCCGCTTGCACCTCGGC |
| 872 | RNASEK | TGGCGTCGCTCCTGTGCTGTG |
| 873 | RPL38 | TGTTGCAGCCTCGGAAAATTG |
| 874 | RPL38 | TCTCTTTCCCTCTAGGTTTGG |
| 875 | RPL38 | CCTCTAGGTTTGGCAGTGAAG |
| 876 | RPL38 | GTCGGGCTGTGAGCAGGAAGT |
| 877 | MYL12B | TTCTTTCTATTGTCTTCCAGG |
| 878 | MYL12B | TATTGTCTTCCAGGCACCATT |
| 879 | MYL12B | GCTAAAGTTCTTTCAGTCATC |
| 880 | PFN1 | CCCATCAGCAGGACTAGCGCT |
| 881 | PFN1 | CTCCTCCTCCAGCGCTAGTCC |
| 882 | PFN1 | TCTTTCCTCCTCCTCCAGCGC |
| 883 | PFN1 | GCATGGATCTTCGTACCAAGA |
| 884 | RPS11 | TCCTCATAATCTGTAGACTGA |
| 885 | RPS11 | TCTTTCCTATCCTTTCAGGCT |
| 886 | RPS11 | CTATCCTTTCAGGCTATTGAG |
| 887 | RPS11 | AGGCTATTGAGGGCACCTACA |
| 888 | RPS11 | TTCTGAGGTTCCCCGCACCTC |

### Example 19—Computation Screening of Guide RNAs for Selection by Essential-Gene Knock-In

The present example describes a method for computationally screening for gRNAs more likely to be suitable for use in targeting essential genes using the selection methods herein that are relevant for different RNA-guided nucleases and variants thereof (e.g., variants of Cas12a, such as Mad7), so long as the RNA-guided nucleases exhibit high cutting efficiency. Cas12b, Cas12e, Cas-Phi, Mad7, and SpyCas9 gRNAs targeting essential genes described preceding examples (GAPDH, TBP, E2F4, G6PD, and KIF11) were selected for this analysis, but a similar process could be applied to identify gRNAs for these RNA-guided nucleases in other essential genes as well. The results of this screening are summarized in tables 18-22, these gRNAs facilitate DNA cleavage within the last 500 bp of the coding sequences of the listed essential genes.

Potential target sequences for each of the essential genes in this analysis (GAPDH, TBP, E2F4, G6PD, and KIF11) were generated by searching for nuclease specific PAMs (ATTN, TTCN, TTN, TTN, and NGG for Cas12b, Cas12e, CasΦ, Mad7, and SpyCas9 respectively) with suitable protospacers mapped to a representative coding region (mRNA-201). Transcripts with its name followed by “−201” were selected as the representative for each gene (e.g., GAPDH-201). Gene information (i.e., coding region) was obtained from GENCODE v.37 gene annotation GTF file. Potential gRNAs were first searched within the genomic regions of target genes in the human reference genome (hg38), and those identified gRNAs with their cut sites within 500 bp of the representative coding region stop site were selected for further analysis. The candidate gRNAs were then aligned to the human reference genome (e.g., hg38) with BWA Aln (maximum mismatch tolerance −n 2). Guides with potential off target binding sites (i.e., aligning to multiple genomic regions; mapping quality MAPQ <30) were filtered out. The resultant gRNAs target essential genes within 500 coding base pairs of a representative stop-codon and have no identical off-target binding sites annotated in the human genome. Thus, gRNAs in Tables 18-22, corresponding to SEQ ID NOs: 889-1885, represent excellent candidate gRNAs for applying the selection methods described herein to GAPDH, TBP, E2F4, G6PD, and

TABLE 18
Cas12b guide RNAs

| SEQ | | Target Domain |
|---|---|---|
| ID NO | Gene | Sequence (DNA) |
| 889 | GAPDH | CCCAGCTCTCATACCATGAGTCC |
| 890 | TBP | TATCCACAGTGAATCTTGGTTGT |
| 891 | TBP | CACTTCGTGCCCGAAACGCCGAA |
| 892 | TBP | TCTCTGACCATTGTAGCGGTTTG |
| 893 | TBP | TAGCGGTTTGCTGCGGTAATCAT |
| 894 | TBP | TCAGTTCTGGGAAAATGGTGTGC |
| 895 | TBP | AGAATATGGTGGGGAGCTGTGAT |
| 896 | TBP | TCCTTCTAGTTATGAGCCAGAGT |
| 897 | TBP | CCTGGTTTAATCTACAGAATGAT |
| 898 | TBP | TTCTCCTTATTTTTGTTTCTGGA |
| 899 | TBP | TTGTTTCTGGAAAAGTTGTATTA |
| 900 | TBP | ATGAAGCATTTGAAAACATCTAG |
| 901 | TBP | TAAAGGGATTCAGGAAGACGACG |
| 902 | TBP | GGCGTTTCGGGCACGAAGTGCAA |
| 903 | TBP | TATTCGGCGTTTCGGGCACGAAG |
| 904 | TBP | AAATAGATCTAACCTTGGGATTA |
| 905 | TBP | TCCCAGAACTGAAAATCAGTGCC |
| 906 | TBP | CTTACGGCTACCTCTTGGCTCCT |
| 907 | TBP | TCTTGCTGCCAGTCTGGACTGTT |
| 908 | TBP | TGAATCTTGAAGTCCAAGAACTT |
| 909 | TBP | TTGGTGGGTGAGCACAAGGCCTT |
| 910 | TBP | CAGACTTAGCTAGTAAATTGTTG |
| 911 | TBP | AACCAGGAAATAACTCTGGCTCA |
| 912 | TBP | TGTAGATTAAACGAGGAAATAAC |
| 913 | TBP | TGGGTTTGATCATTCTGTAGATT |
| 914 | TBP | CTGCTCTGACTTTAGCACCTAAG |
| 915 | TBP | CGTCGTCTTCCTGAATCCCTTTA |
| 916 | E2F4 | TAGTGAGTGGCGGCCCTGGGACT |
| 917 | E2F4 | CCAGAGTGCATGAGCTCGGAGCT |
| 918 | E2F4 | TATCTAGAACCTGGACGAGAGTG |
| 919 | E2F4 | CCTGGACTTCTGCACTGCCAGGG |
| 920 | E2F4 | CTGACAGCTCTTTGGGGAGTTCC |
| 921 | G6PD | AGCTGGAGAAGCCCAAGCCCATC |
| 922 | G6PD | TCACCCCACTGCTGCACCAGATT |
| 923 | KIF11 | ATGAAGATAAATTGATAGCACAA |
| 924 | KIF11 | ATAGCACAAAATCTAGAACTTAA |
| 925 | KIF11 | GTTTGACTAAGCTTAATTGCTTT |
| 926 | KIF11 | CTTTCTGGAACAGGATCTGAAAC |
| 927 | KIF11 | ATACCCATCAACACTGGTAAGAA |
| 928 | KIF11 | TTCATCAATTGGCGGGGTTCCAT |
| 929 | KIF11 | GCGGGGTTCCATTTTTCCAGGTA |
| 930 | KIF11 | TCCCGCCTTAAATCCACAGCATA |
| 931 | KIF11 | ACACACTGGAGAGGTCTAAAGTG |
| 932 | KIF11 | CCTCTGCGAGCCCAGATCAACCT |
| 933 | KIF11 | AGTTCTAGATTTTGTGCTATCAA |
| 934 | KIF11 | TTATGGTTTCATTAAGTTCTAGA |
| 935 | KIF11 | AGCTTAGTCAAACCAATTTTTAT |
| 936 | KIF11 | CTCTTTTAAAGTACCTGTTGGGA |
| 937 | KIF11 | TATTTCTCTTTTAAAGTACCTGT |
| 938 | KIF11 | ACAGCTCAGGCTGTTTCCTTTTC |
| 939 | KIF11 | TCTCTTCTTTGTTGTTTTCTGAA |
| 940 | KIF11 | ACCGGAATTGTCTCTTCTTTGTT |
| 941 | KIF11 | ATGAACAATCCACACCAGCATCT |
| 942 | KIF11 | AAGGTTGATCTGGGCTCGCAGAG |
| 943 | KIF11 | CCAACCCCCAAGTGAATTAAAGG |
| — | — | — |

TABLE 19
Cas12e guide RNAs

| | SEQ | | Target Domain |
|---|---|---|---|
| | ID NO | Gene | Sequence (DNA) |
| | 944 | GAPDH | TCTTCTAGGTATGAGAACGAA |
| | 945 | GAPDH | CGAGCTCTCATACCATGAGTC |
| | 946 | TBP | TGCCCGAAACGCCGAATATAA |
| | 947 | TBP | CTCTGACCATTGTAGCGGTTT |
| | 948 | TBP | GTTCTGGGAAAATGGTGTGCA |
| | 949 | TBP | GGGAAAATGGTGTGCACAGGA |
| | 950 | TBP | TTTCCCTAGTGAAGAACAGTC |
| | 951 | TBP | CTAGTGAAGAACAGTCCAGAC |
| | 952 | TBP | AGCTAAGTTCTTGGACTTCAA |
| | 953 | TBP | TGGACTTCAAGATTCAGAATA |
| | 954 | TBP | AGATTCAGAATATGGTGGGGA |
| | 955 | TBP | GAATATGGTGGGGAGCTGTGA |
| | 956 | TBP | TATAAGGTTAGAAGGCCTTGT |
| | 957 | TBP | TTCTAGTTATGAGCCAGAGTT |
| | 958 | TBP | AGTTATGAGCCAGAGTTATTT |
| | 959 | TBP | TGGTTTAATCTACAGAATGAT |
| | 960 | TBP | CCTTATTTTTGTTTCTGGAAA |
| | 961 | TBP | GGAAAAGTTGTATTAACAGGT |
| | 962 | TBP | TAGGTGCTAAAGTCAGAGCAG |
| | 963 | TBP | AAAGGGATTCAGGAAGACGAC |
| | 964 | TBP | GGCACGAAGTGCAATGGTCTT |
| | 965 | TBP | GCGTTTCGGGCACGAAGTGCA |
| | 966 | TBP | TGGCTCTCTTATCCTCATGAT |
| | 967 | TBP | CAGAACTGAAAATCAGTGCCG |
| | 968 | TBP | TACGGCTACCTCTTGGCTCCT |
| | 969 | TBP | TGCTGCCAGTCTGGACTGTTC |
| | 970 | TBP | GTACAACTCTAGCATATTTTC |
| | 971 | TBP | GAATCTTGAAGTCCAAGAACT |
| | 972 | TBP | CATCACAGCTCCCCACCATAT |
| | 973 | TBP | AACCTTATAGGAAACTTCACA |
| | 974 | TBP | GACTTACCTACTAAATTGTTG |
| | 975 | TBP | GTAGATTAAACCAGGAAATAA |
| | 976 | TBP | GGGTTTGATCATTCTGTAGAT |
| | 977 | TBP | AGAAACAAAAATAAGGAGAAC |
| | 978 | TBP | TGTTACAACTTACCTGTTAAT |
| | 979 | TBP | GCTCTGACTTTAGCACCTAAG |
| | 980 | TBP | TAAATTTCTGCTCTGACTTTA |
| | 981 | TBP | AATGCTTCATAAATTTCTGCT |
| | 982 | TBP | TGAATCCCTTTAGAATAGGGT |
| | 983 | E2F4 | CTCCCACTGGGCCCAACAACA |
| | 984 | E2F4 | GCCCTGCTGGACAGCAGCAGC |
| | 985 | E2F4 | TCCGGACCCAACCCTTCTACC |
| | 986 | E2F4 | ACCTCCTTTGAGCCCATCAAG |
| | 987 | E2F4 | TGTTTTTCAGTTTTGGAACTC |
| | 988 | E2F4 | GTTTTGGAACTCCCCAAAGAG |
| | 989 | E2F4 | CAGAGTGCATGAGCTCGGAGC |
| | 990 | E2F4 | TCTTTCTCCACCCCCGGGAGA |
| | 991 | E2F4 | CCACCCCCGGGAGACCACGAT |
| | 992 | E2F4 | GCACTGCCAGGGACAGCAGTG |
| | 993 | E2F4 | CTGGACTTCTGCACTGCCAGG |
| | 994 | E2F4 | GACAGCTCTTTGGGGAGTTCC |
| | 995 | E2F4 | GAGGACATCAACTCCTCCAGC |
| | 996 | E2F4 | AGGGCCACCCACCTTCTGAGG |
| | 997 | E2F4 | CTCTCGTCCAGGTTGTAGATA |
| | 998 | G6PD | CCCACTTGTAGGTGCCCTCAT |
| | 999 | G6PD | TCAGCTCGTCTGCCTCCGTGG |
| | 1000 | G6PD | TCACCTGCCATAAATATAGGG |
| | 1001 | G6PD | CCAGCTCAATCTGGTGCAGCA |
| | 1002 | G6PD | CTGTAGGGCACCTTGTATCTG |
| | 1003 | G6PD | TGGTCATCATCTTGGTGTACA |
| | 1004 | G6PD | GGGCCTTGCCGCAGCGCAGGA |
| | 1005 | G6PD | AGTATGAGGGCACCTACAAGT |
| | 1006 | G6PD | CCCCACTGCTGCACCAGATTG |
| | 1007 | G6PD | GCGGGAGCCAGATGCACTTCG |
| | 1008 | G6PD | ACCCCGAGGAGTCGGAGCTGG |
| | 1009 | G6PD | TCAACCCCGAGGAGTCGGAGC |
| | 1010 | G6PD | ACCAGCAGTGCAAGCGCAACG |
| | 1011 | G6PD | ATGATGTGGCCGGCGACATCT |
| | 1012 | G6PD | TCCTGCGCTGCGGCAAGGCCC |
| | 1013 | G6PD | GCCACGTAGGGGTGCCCTTCA |
| | 1014 | KIF11 | GGAACAGGATCTGAAACTGGA |
| | 1015 | KIF11 | GAAAACAACAAAGAAGAGACA |
| | 1016 | KIF11 | TCTTTTAGGATGTGGATGTAG |
| | 1017 | KIF11 | TTTAGGATGTGGATGTAGAAG |
| | 1018 | KIF11 | GGGGCAGTATACTGAAGAACC |
| | 1019 | KIF11 | TCAATTGGCGGGGTTCCATTT |
| | 1020 | KIF11 | CGCCTTAAATCCACAGCATAA |
| | 1021 | KIF11 | AGATTTTGTGCTATCAATTTA |
| | 1022 | KIF11 | TTAAGTTCTAGATTTTGTGCT |
| | 1023 | KIF11 | AGAAAGCAATTAAGCTTAGTC |
| | 1024 | KIF11 | GATCCTGTTCCAGAAAGCAAT |
| | 1025 | KIF11 | CTTTTAAAGTACCTGTTGGGA |
| | 1026 | KIF11 | ATTTCTCTTTTAAAGTACCTG |
| | 1027 | KIF11 | TCTGTGGTGTCGTACCTTTAA |
| | 1028 | KIF11 | TACCAGTGTTGATGGGTATAA |
| | 1029 | KIF11 | GTTCTTACCAGTGTTGATGGG |
| | 1030 | KIF11 | CGTGGTTCAGTTCTTACCAGT |
| | 1031 | KIF11 | GCTGATCAAGGAGATGTTCAC |
| | 1032 | KIF11 | TTTTCAGCTGATCAAGGAGAT |
| | 1033 | KIF11 | GAACAGTTTAGCATCATTAAC |
| | 1034 | KIF11 | TTGTTGTTTTCTGAACAGTTT |
| | 1035 | KIF11 | GTATACTGCCCCAGAACTGCC |
| | 1036 | KIF11 | TCAGTATACTGCCCCAGAACT |
| | 1037 | KIF11 | ATGTGATTTTTTATGCTGTGG |
| | 1038 | KIF11 | TTGTCTTTTCCATGTGATTTT |
| | 1039 | KIF11 | ACTTTAGACCTCTCCAGTGTG |
| | 1040 | KIF11 | TCCACTTTAGACCTCTCCAGT |
| — | — | — | |

TABLE 20
Cas-Phi guide RNAs

| SEQ | | Target Domain |
|---|---|---|
| ID NO | Gene | Sequence (DNA) |
| 1041 | GAPDH | TGCAGACCACAGTCCATGCCA |
| 1042 | GAPDH | GCAGACCACAGTCCATGCCAT |
| 1043 | GAPDH | CAGACCACAGTCCATGCCATC |
| 1044 | GAPDH | TCATCTTCTAGGTATGACAAC |
| 1045 | GAPDH | CATCTTCTAGGTATGACAACG |
| 1046 | GAPDH | ATCTTCTAGGTATGACAACGA |
| 1047 | GAPDH | TAGGTATGACAACGAATTTGG |
| 1048 | GAPDH | CCCAGCTCTCATACCATGAGT |
| 1049 | TBP | TATCCACAGTGAATCTTGGTT |
| 1050 | TBP | GTTGTAAACTTGACCTAAAGA |
| 1051 | TBP | TAAACTTGACCTAAAGACCAT |
| 1052 | TBP | ACCTAAAGACCATTGCACTTC |
| 1053 | TBP | CACTTCGTGCCCGAAACGCCG |
| 1054 | TBP | GTGCCCGAAACGCCGAATATA |
| 1055 | TBP | TCTCTGACCATTGTAGCGGTT |
| 1056 | TBP | TAGCGGTTTGCTGCGGTAATC |
| 1057 | TBP | GCTGCGGTAATCATGAGGATA |
| 1058 | TBP | CTGCGGTAATCATGAGGATAA |
| 1059 | TBP | TCAGTTCTGGGAAAATGGTGT |
| 1060 | TBP | CAGTTCTGGGAAAATGGTGTG |
| 1061 | TBP | AGTTCTGGGAAAATGGTGTGC |
| 1062 | TBP | TGGGAAAATGGTGTGCACAGG |
| 1063 | TBP | TTTCCTTTCCCTAGTGAAGAA |
| 1064 | TBP | TTCCTTTCCCTAGTGAAGAAC |
| 1065 | TBP | TCCTTTCCCTAGTGAAGAACA |
| 1066 | TBP | CCTTTCCCTAGTGAAGAACAG |
| 1067 | TBP | CTTTCCCTAGTGAAGAACAGT |
| 1068 | TBP | CCCTAGTGAAGAACAGTCCAG |
| 1069 | TBP | CCTAGTGAAGAACAGTCCAGA |
| 1070 | TBP | TACAGAAGTTGGGTTTTCCAG |
| 1071 | TBP | GGTTTTCCAGCTAAGTTCTTG |
| 1072 | TBP | TCCAGCTAAGTTCTTGGACTT |
| 1073 | TBP | CCAGCTAAGTTCTTGGACTTC |
| 1074 | TBP | CAGCTAAGTTCTTGGACTTCA |
| 1075 | TBP | TTGGACTTCAAGATTCAGAAT |
| 1076 | TBP | GAGTTCAAGATTCAGAATATG |
| 1077 | TBP | AAGATTCAGAATATGGTGGGG |
| 1078 | TBP | AGAATATGGTGGGGAGCTGTG |
| 1079 | TBP | CCTATAAGGTTAGAAGGCCTT |
| 1080 | TBP | CTATAAGGTTAGAAGGCCTTG |
| 1081 | TBP | TGCTCACCCACCAACAATTTA |
| 1082 | TBP | TTGCAATTTTCCTTCTAGTTA |
| 1083 | TBP | TGCAATTTTCCTTCTAGTTAT |
| 1084 | TBP | GCAATTTTCCTTCTAGTTATG |
| 1085 | TBP | CAATTTTCCTTCTAGTTATGA |
| 1086 | TBP | TCCTTCTAGTTATGAGCCAGA |
| 1087 | TBP | CCTTCTAGTTATGAGCCAGAG |
| 1088 | TBP | CTTCTAGTTATGAGCCAGAGT |
| 1089 | TBP | TAGTTATGAGCCAGAGTTATT |
| 1090 | TBP | TGAGCCAGAGTTATTTCCTGG |
| 1091 | TBP | CCTGGTTTAATCTACAGAATG |
| 1092 | TBP | CTGGTTTAATCTACAGAATGA |
| 1093 | TBP | AATCTACAGAATGATCAAACC |
| 1094 | TBP | ATCTACAGAATGATCAAACCC |
| 1095 | TBP | TTCTCCTTATTTTTGTTTCTG |
| 1096 | TBP | TCCTTATTTTTGTTTCTGGAA |
| 1097 | TBP | TTTTTGTTTCTGGAAAAGTTG |
| 1098 | TBP | TTGTTTCTGGAAAAGTTGTAT |
| 1099 | TBP | TGTTTCTGGAAAAGTTGTATT |
| 1100 | TBP | GTTTCTGGAAAAGTTGTATTA |
| 1101 | TBP | TTTCTGGAAAAGTTGTATTAA |
| 1102 | TBP | CTGGAAAAGTTGTATTAACAG |
| 1103 | TBP | TGGAAAAGTTGTATTAACAGG |
| 1104 | TBP | TCTTCTTAGGTGCTAAAGTCA |
| 1105 | TBP | TTAGGTGCTAAAGTCAGAGCA |
| 1106 | TBP | GGTGCTAAAGTCAGAGCAGAA |
| 1107 | TBP | TAAAGGGATTCAGGAAGACGA |
| 1108 | TBP | GGTCAAGTTTAGAACCAAGAT |
| 1109 | TBP | AGGTCAAGTTTACAACCAAGA |
| 1110 | TBP | GGGCACGAAGTGCAATGGTCT |
| 1111 | TBP | CGGGCACGAAGTGCAATGGTC |
| 1112 | TBP | GGCGTTTCGGGCACGAAGTGC |
| 1113 | TBP | TATTCGGCGTTTCGGGCACGA |
| 1114 | TBP | GGATTATATTCGGCGTTTCGG |
| 1115 | TBP | AAATAGATCTAACCTTGGGAT |
| 1116 | TBP | TCCTCATGATTACCGCAGCAA |
| 1117 | TBP | GTGGCTCTCTTATCCTCATGA |
| 1118 | TBP | CCAGAACTGAAAATCAGTGCC |
| 1119 | TBP | CCCAGAACTGAAAATCAGTGC |
| 1120 | TBP | TCCCAGAACTGAAAATCAGTG |
| 1121 | TBP | GCTCCTGTGCACACCATTTTC |
| 1122 | TBP | CGGCTACCTCTTGGCTCCTGT |
| 1123 | TBP | TTACGGCTACCTCTTGGCTCC |
| 1124 | TBP | CTTACGGCTACCTCTTGGCTC |
| 1125 | TBP | CTGCCAGTCTGGACTGTTCTT |
| 1126 | TBP | TTGCTGCCAGTCTGGACTGTT |
| 1127 | TBP | CTTGCTGCCAGTCTGGACTGT |
| 1128 | TBP | TCTTGCTGCCAGTCTGGACTG |
| 1129 | TBP | TGTACAACTCTAGCATATTTT |
| 1130 | TBP | GCTGGAAAACCCAACTTCTGT |
| 1131 | TBP | AAGTCCAAGAACTTAGCTGGA |
| 1132 | TBP | TGAATCTTGAAGTCCAAGAAC |
| 1133 | TBP | ACATCACAGCTCCCCACCATA |
| 1134 | TBP | TAACCTTATAGGAAACTTCAC |
| 1135 | TBP | GTGGGTGAGCACAAGGCCTTC |
| 1136 | TBP | TTGGTGGGTGAGCACAAGGCC |
| 1137 | TBP | CCTACTAAATTGTTGGTGGGT |
| 1138 | TBP | AGACTTAGCTAGTAAATTGTT |
| 1139 | TBP | CAGACTTAGCTAGTAAATTGT |
| 1140 | TBP | AACCAGGAAATAACTCTGGCT |
| 1141 | TBP | TGTAGATTAAACCAGGAAATA |
| 1142 | TBP | ATCATTCTGTAGATTAAACCA |
| 1143 | TBP | GATCATTCTGTAGATTAAACC |
| 1144 | TBP | TGGGTTTGATCATTCTGTAGA |
| 1145 | TBP | CAGAAACAAAAATAAGGAGAA |
| 1146 | TBP | CCAGAAACAAAAATAAGGAGA |
| 1147 | TBP | TCCAGAAACAAAAATAAGGAG |
| 1148 | TBP | ATACAACTTTTCCAGAAACAA |
| 1149 | TBP | CCTGTTAATACAACTTTTCCA |
| 1150 | TBP | CAACTTACCTGTTAATACAAC |
| 1151 | TBP | CTGTTACAACTTACCTGTTAA |
| 1152 | TBP | TGCTCTGACTTTAGCACCTAA |
| 1153 | TBP | CTGCTCTGACTTTAGCACCTA |
| 1154 | TBP | ATAAATTTCTGCTCTGACTTT |
| 1155 | TBP | AAATGCTTCATAAATTTCTGC |
| 1156 | TBP | CAAATGCTTCATAAATTTCTG |
| 1157 | TBP | TCAAATGCTTCATAAATTTCT |
| 1158 | TBP | CTGAATCCCTTTAGAATAGGG |
| 1159 | TBP | CGTCGTCTTCCTGAATCCCTT |
| 1160 | E2F4 | GGGGGCTATCATTGTAGTGAG |
| 1161 | E2F4 | GGGGCTATCATTGTAGTGAGT |
| 1162 | E2F4 | TAGTGAGTGGCGGCCCTGGGA |
| 1163 | E2F4 | ACTCCCACTGGGCCCAACAAC |
| 1164 | E2F4 | TGCCCTGCTGGACAGCAGCAG |
| 1165 | E2F4 | GTCCGGACCCAACCCTTCTAC |
| 1166 | E2F4 | TACCTCCTTTGAGCCCATCAA |
| 1167 | E2F4 | GAGCCCATCAAGGCAGACCCC |
| 1168 | E2F4 | AGCCCATCAAGGCAGACCCCA |
| 1169 | E2F4 | CTTGTTTTTCAGTTTTGGAAC |
| 1170 | E2F4 | TTTTTCAGTTTTGGAACTCCC |
| 1171 | E2F4 | TTCAGTTTTGGAACTCCCCAA |
| 1172 | E2F4 | TCAGTTTTGGAACTCCCCAAA |
| 1173 | E2F4 | CAGTTTTGGAACTCCCCAAAG |
| 1174 | E2F4 | AGTTTTGGAACTCCCCAAAGA |
| 1175 | E2F4 | TGGAACTCCCCAAAGAGCTGT |
| 1176 | E2F4 | GGAACTCCCCAAAGAGCTGTC |
| 1177 | E2F4 | CCAGAGTGCATGAGCTCGGAG |
| 1178 | E2F4 | GCCCCTCTGCTTCGTCTTTCT |
| 1179 | E2F4 | CCCCTCTGCTTCGTCTTTCTC |
| 1180 | E2F4 | GTCTTTCTCCACCCCCGGGAG |
| 1181 | E2F4 | CTCCACCCCCGGGAGACCACG |
| 1182 | E2F4 | TCCACCCCCGGGAGACCACGA |
| 1183 | E2F4 | TATCTACAACCTGGAGGAGAG |
| 1184 | E2F4 | GATGTGCCTGTTCTCAACCTC |
| 1185 | E2F4 | ATGTGCCTGTTCTCAACCTCT |
| 1186 | E2F4 | TGCACTGCCAGGGACAGCAGT |
| 1187 | E2F4 | CCTGGACTTCTGCACTGCCAG |
| 1188 | E2F4 | CTATCAGTCCCAGGGCCGCCA |
| 1189 | E2F4 | GGCCCAGTGGGAGTGAACTGA |
| 1190 | E2F4 | TTGGGCCCAGTGGGAGTGAAC |
| 1191 | E2F4 | GGTCCGGACGAACTGCTGCTG |
| 1192 | E2F4 | ATGGGCTCAAAGGAGGTAGAA |
| 1193 | E2F4 | TGACAGCTCTTTGGGGAGTTC |
| 1194 | E2F4 | CTGACAGCTCTTTGGGGAGTT |
| 1195 | E2F4 | TGAGGACATCAACTCCTCCAG |
| 1196 | E2F4 | CAGGGCCACCCACCTTCTGAG |
| 1197 | E2F4 | TAGATATAATCGTGGTCTCCC |
| 1198 | E2F4 | ACTCTCGTCCAGGTTGTAGAT |
| 1199 | G6PD | TGGGGGTTCACCCACTTGTAG |
| 1200 | G6PD | ACCCACTTGTAGGTGCCCTCA |
| 1201 | G6PD | TAGGTGCCCTCATACTGGAAA |
| 1202 | G6PD | ATCAGCTCGTCTGCCTCCGTG |
| 1203 | G6PD | CCTCACCTGCCATAAATATAG |
| 1204 | G6PD | CTCACCTGCCATAAATATAGG |
| 1205 | G6PD | GGCTTCTCCAGCTCAATCTGG |
| 1206 | G6PD | TCCAGCTCAATCTGGTGCAGC |
| 1207 | G6PD | TCTGTAGGGCACCTTGTATCT |
| 1208 | G6PD | TATCTGTTGCCGTAGGTCAGG |
| 1209 | G6PD | CCGTAGGTCAGGTCCAGCTCC |
| 1210 | G6PD | AAGAACATGCCCGGCTTCTTG |
| 1211 | G6PD | TTGGTCATCATCTTGGTGTAC |
| 1212 | G6PD | GTCATCATCTTGGTGTACACG |
| 1213 | G6PD | GTGTACACGGCCTCGTTGGGC |
| 1214 | G6PD | GGCTGCACGCGGATCACCAGC |
| 1215 | G6PD | CGCTTGCACTGCTGGTGGAAG |
| 1216 | G6PD | CACTGCTGGTGGAAGATGTCG |
| 1217 | G6PD | CGCTCGTTCAGGGCCTTGCCG |
| 1218 | G6PD | AGGGCCTTGCCGCAGCGCAGG |
| 1219 | G6PD | CCGCAGCGCAGGATGAAGGGC |
| 1220 | G6PD | CAGTATGAGGGCACCTACAAG |
| 1221 | G6PD | CCAGTATGAGGGCACCTACAA |
| 1222 | G6PD | AGCTGGAGAAGCCCAAGCCCA |
| 1223 | G6PD | ACCCCACTGCTGCACCAGATT |
| 1224 | G6PD | CACCCCACTGCTGCACCAGAT |
| 1225 | G6PD | TCACCCCACTGCTGCACCAGA |
| 1226 | G6PD | TGCGGGAGCCAGATGCACTTC |
| 1227 | G6PD | AACCCCGAGGAGTCGGAGCTG |
| 1228 | G6PD | TTCAACCCCGAGGAGTCGGAG |
| 1229 | G6PD | CACCAGCAGTGCAAGCGCAAC |
| 1230 | G6PD | CATGATGTGGCCGGCGACATC |
| 1231 | G6PD | ATCCTGCGCTGCGGCAAGGCC |
| 1232 | G6PD | CGCCACGTAGGGGTGCCCTTC |
| 1233 | G6PD | CCGCCACGTAGGGGTGCCCTT |
| 1234 | KIF11 | ATGAAGATAAATTGATAGCAC |
| 1235 | KIF11 | ATAGCACAAAATCTAGAACTT |
| 1236 | KIF11 | ATGAAACCATAAAAATTGGTT |
| 1237 | KIF11 | GTTTGACTAAGCTTAATTGCT |
| 1238 | KIF11 | GACTAAGCTTAATTGCTTTCT |
| 1239 | KIF11 | ACTAAGCTTAATTGCTTTCTG |
| 1240 | KIF11 | ATTGCTTTCTGGAACAGGATC |
| 1241 | KIF11 | CTTTCTGGAACAGGATCTGAA |
| 1242 | KIF11 | CTGGAACAGGATCTGAAACTG |
| 1243 | KIF11 | TGGAACAGGATCTGAAACTGG |
| 1244 | KIF11 | TCTAATGTCCGTTAAAGGTAC |
| 1245 | KIF11 | AAGGTACGACACCACAGAGGA |
| 1246 | KIF11 | TTTATACCCATCAACACTGGT |
| 1247 | KIF11 | ATACCCATCAACACTGGTAAG |
| 1248 | KIF11 | TACCCATCAACACTGGTAAGA |
| 1249 | KIF11 | ATCAGCTGAAAAGGAAACAGC |
| 1250 | KIF11 | ATGATGCTAAACTGTTCAGAA |
| 1251 | KIF11 | AGAAAACAACAAAGAAGAGAC |
| 1252 | KIF11 | CTTCTTTTAGGATGTGGATGT |
| 1253 | KIF11 | TTCTTTTAGGATGTGGATGTA |
| 1254 | KIF11 | TTTTAGGATGTGGATGTAGAA |
| 1255 | KIF11 | TAGGATGTGGATGTAGAAGAG |
| 1256 | KIF11 | AGGATGTGGATGTAGAAGAGG |
| 1257 | KIF11 | GGATGTGGATGTAGAAGAGGC |
| 1258 | KIF11 | TGGGGCAGTATACTGAAGAAC |
| 1259 | KIF11 | TTCATCAATTGGCGGGGTTCC |
| 1260 | KIF11 | ATCAATTGGCGGGGTTCCATT |
| 1261 | KIF11 | GCGGGGTTCCATTTTTCCAGG |
| 1262 | KIF11 | TCCCGCCTTAAATCCACAGCA |
| 1263 | KIF11 | CCCGCCTTAAATCCACAGCAT |
| 1264 | KIF11 | CCGCCTTAAATCCACAGCATA |
| 1265 | KIF11 | AATCCACAGCATAAAAAATCA |
| 1266 | KIF11 | ACACACTGGAGAGGTCTAAAG |
| 1267 | KIF11 | GTTACAAAGAGCAGATTACCT |
| 1268 | KIF11 | CAAAGAGCAGATTACCTCTGC |
| 1269 | KIF11 | CCTCTGCGAGCCCAGATCAAC |
| 1270 | KIF11 | TAGATTTTGTGCTATCAATTT |
| 1271 | KIF11 | AGTTCTAGATTTTGTGCTATC |
| 1272 | KIF11 | ATTAAGTTCTAGATTTTGTGC |
| 1273 | KIF11 | CATTAAGTTCTAGATTTTGTG |
| 1274 | KIF11 | TGGTTTCATTAAGTTCTAGAT |
| 1275 | KIF11 | ATGGTTTCATTAAGTTCTAGA |
| 1276 | KIF11 | TATGGTTTCATTAAGTTCTAG |
| 1277 | KIF11 | TTATGGTTTCATTAAGTTCTA |
| 1278 | KIF11 | GTCAAACCAATTTTTATGGTT |
| 1279 | KIF11 | AGCTTAGTCAAACCAATTTTT |
| 1280 | KIF11 | CAGAAAGCAATTAAGCTTAGT |
| 1281 | KIF11 | AGATCCTGTTCCAGAAAGCAA |
| 1282 | KIF11 | CAGATCCTGTTCCAGAAAGCA |
| 1283 | KIF11 | GGATATCCAGTTTCAGATCCT |
| 1284 | KIF11 | AAGTACCTGTTGGGATATCCA |
| 1285 | KIF11 | AAAGTACCTGTTGGGATATCC |
| 1286 | KIF11 | TAAAGTACCTGTTGGGATATC |
| 1287 | KIF11 | TCTTTTAAAGTACCTGTTGGG |
| 1288 | KIF11 | CTCTTTTAAAGTACCTGTTGG |
| 1289 | KIF11 | TATTTCTCTTTTAAAGTACCT |
| 1290 | KIF11 | CTCTGTGGTGTCGTACCTTTA |
| 1291 | KIF11 | CCTCTGTGGTGTCGTACCTTT |
| 1292 | KIF11 | TCCTCTGTGGTGTCGTACCTT |
| 1293 | KIF11 | ATGGGTATAAATAACTTTTCC |
| 1294 | KIF11 | CCAGTGTTGATGGGTATAAAT |
| 1295 | KIF11 | TTACCAGTGTTGATGGGTATA |
| 1296 | KIF11 | AGTTCTTACCAGTGTTGATGG |
| 1297 | KIF11 | ACGTGGTTCAGTTCTTACCAG |
| 1298 | KIF11 | AGCTGATCAAGGAGATGTTCA |
| 1299 | KIF11 | CAGCTGATCAAGGAGATGTTC |
| 1300 | KIF11 | TCAGCTGATCAAGGAGATGTT |
| 1301 | KIF11 | CTTTTCAGCTGATCAAGGAGA |
| 1302 | KIF11 | CCTTTTCAGCTGATCAAGGAG |
| 1303 | KIF11 | ACAGCTCAGGCTGTTTCCTTT |
| 1304 | KIF11 | GCATCATTAACAGCTCAGGCT |
| 1305 | KIF11 | AGCATCATTAACAGCTCAGGC |
| 1306 | KIF11 | TGAACAGTTTAGCATCATTAA |
| 1307 | KIF11 | CTGAACAGTTTAGCATCATTA |
| 1308 | KIF11 | TCTGAACAGTTTAGCATCATT |
| 1309 | KIF11 | TTTTCTGAACAGTTTAGCATC |
| 1310 | KIF11 | TTGTTTTCTGAACAGTTTAGC |
| 1311 | KIF11 | TTTGTTGTTTTCTGAACAGTT |
| 1312 | KIF11 | TCTCTTCTTTGTTGTTTTCTG |
| 1313 | KIF11 | CCGGAATTGTCTCTTCTTTGT |
| 1314 | KIF11 | ACCGGAATTGTCTCTTCTTTG |
| 1315 | KIF11 | AATTTACCGGAATTGTCTCTT |
| 1316 | KIF11 | AAATTTACCGGAATTGTCTCT |
| 1317 | KIF11 | AGTATACTGCCCCAGAACTGC |
| 1318 | KIF11 | TTCAGTATACTGCCCCAGAAC |
| 1319 | KIF11 | GAGGTTCTTCAGTATACTGCC |
| 1320 | KIF11 | ACTTAGAGGTTCTTCAGTATA |
| 1321 | KIF11 | ATGAACAATCCACACCAGCAT |
| 1322 | KIF11 | TCTGATATGACATACCTGGAA |
| 1323 | KIF11 | CATGTGATTTTTTATGCTGTG |
| 1324 | KIF11 | CCATGTGATTTTTTATGCTGT |
| 1325 | KIF11 | TCCATGTGATTTTTTATGCTG |
| 1326 | KIF11 | TCTTTTCCATGTGATTTTTTA |
| 1327 | KIF11 | GTCTTTTCCATGTGATTTTTT |
| 1328 | KIF11 | TTTGTCTTTTCCATGTGATTT |
| 1329 | KIF11 | CTTTGTCTTTTCCATGTGATT |
| 1330 | KIF11 | TCTTTGTCTTTTCCATGTGAT |
| 1331 | KIF11 | ATGCCTCTGTTTTCTTTGTCT |
| 1332 | KIF11 | GACCTCTCCAGTGTGTTAATG |
| 1333 | KIF11 | AGACCTCTCCAGTGTGTTAAT |
| 1334 | KIF11 | CACTTTAGACCTCTCCAGTGT |
| 1335 | KIF11 | TTCCACTTTAGACCTCTCCAG |
| 1336 | KIF11 | CTTCCACTTTAGACCTCTCCA |
| 1337 | KIF11 | TAACCAAGTGCTCTGTAGTTT |
| 1338 | KIF11 | GTAACCAAGTGCTCTGTAGTT |
| 1339 | KIF11 | ATCTGGGCTCGCAGAGGTAAT |
| 1340 | KIF11 | AAGGTTGATCTGGGCTCGCAG |
| 1341 | KIF11 | CCAACCCCCAAGTGAATTAAA |
| — | — | — |

TABLE 21
Mad7 guide RNAs

| SEQ | | Target Domain |
|---|---|---|
| ID NO | Gene | Sequence (DNA) |
| 1342 | GAPDH | TGCAGACCACAGTCCATGCCA |
| 1343 | GAPDH | GCAGACCACAGTCCATGCCAT |
| 1344 | GAPDH | CAGACCACAGTCCATGCCATC |
| 1345 | GAPDH | TCATCTTCTAGGTATGACAAC |
| 1346 | GAPDH | CATCTTCTAGGTATGACAACG |
| 1347 | GAPDH | ATCTTCTAGGTATGACAACGA |
| 1348 | GAPDH | TAGGTATGACAACGAATTTGG |
| 1349 | GAPDH | CCCAGCTCTCATACCATGAGT |
| 1350 | TBP | TATCCACAGTGAATCTTGGTT |
| 1351 | TBP | GTTGTAAACTTGACCTAAAGA |
| 1352 | TBP | TAAACTTGACCTAAAGACCAT |
| 1353 | TBP | ACCTAAAGACCATTGCACTTC |
| 1354 | TBP | CACTTCGTGCCCGAAACGCCG |
| 1355 | TBP | GTGCCCGAAACGCCGAATATA |
| 1356 | TBP | TCTCTGACCATTGTAGCGGTT |
| 1357 | TBP | TAGCGGTTTGCTGCGGTAATC |
| 1358 | TBP | GCTGCGGTAATCATGAGGATA |
| 1359 | TBP | CTGCGGTAATCATGAGGATAA |
| 1360 | TBP | TCAGTTCTGGGAAAATGGTGT |
| 1361 | TBP | CAGTTCTGGGAAAATGGTGTG |
| 1362 | TBP | AGTTCTGGGAAAATGGTGTGC |
| 1363 | TBP | TGGGAAAATGGTGTGCACAGG |
| 1364 | TBP | TTTCCTTTCCCTAGTGAAGAA |
| 1365 | TBP | TTCCTTTCCCTAGTGAAGAAC |
| 1366 | TBP | TCCTTTCCCTAGTGAAGAACA |
| 1367 | TBP | CCTTTCCCTAGTGAAGAACAG |
| 1368 | TBP | CTTTCCCTAGTGAAGAACAGT |
| 1369 | TBP | CCCTAGTGAAGAACAGTCGAG |
| 1370 | TBP | CCTAGTGAAGAACAGTCCAGA |
| 1371 | TBP | TACAGAAGTTGGGTTTTCCAG |
| 1372 | TBP | GGTTTTCCAGCTAAGTTCTTG |
| 1373 | TBP | TCCAGCTAAGTTCTTGGACTT |
| 1374 | TBP | CCAGCTAAGTTCTTGGACTTC |
| 1375 | TBP | CAGCTAAGTTCTTGGACTTCA |
| 1376 | TBP | TTGGACTTCAAGATTCAGAAT |
| 1377 | TBP | GACTTCAAGATTCAGAATATG |
| 1378 | TBP | AAGATTCAGAATATGGTGGGG |
| 1379 | TBP | AGAATATGGTGGGGAGCTGTG |
| 1380 | TBP | CCTATAAGGTTAGAAGGCCTT |
| 1381 | TBP | CTATAAGGTTAGAAGGCCTTG |
| 1382 | TBP | TGCTCACCCACCAACAATTTA |
| 1383 | TBP | TTGCAATTTTCCTTCTAGTTA |
| 1384 | TBP | TGCAATTTTCCTTCTAGTTAT |
| 1385 | TBP | GCAATTTTCCTTCTAGTTATG |
| 1386 | TBP | CAATTTTCCTTCTAGTTATGA |
| 1387 | TBP | TCCTTCTAGTTATGAGCCAGA |
| 1388 | TBP | CCTTCTAGTTATGAGCCAGAG |
| 1389 | TBP | CTTCTAGTTATGAGCCAGAGT |
| 1390 | TBP | TAGTTATGAGCCAGAGTTATT |
| 1391 | TBP | TGAGCCAGAGTTATTTCCTGG |
| 1392 | TBP | CCTGGTTTAATCTACAGAATG |
| 1393 | TBP | CTGGTTTAATCTACAGAATGA |
| 1394 | TBP | AATCTACAGAATGATCAAACC |
| 1395 | TBP | ATCTACAGAATGATCAAACCC |
| 1396 | TBP | TTCTCCTTATTTTTGTTTCTG |
| 1397 | TBP | TCCTTATTTTTGTTTCTGGAA |
| 1398 | TBP | TTTTTGTTTCTGGAAAAGTTG |
| 1399 | TBP | TTGTTTCTGGAAAAGTTGTAT |
| 1400 | TBP | TGTTTCTGGAAAAGTTGTATT |
| 1401 | TBP | GTTTCTGGAAAAGTTGTATTA |
| 1402 | TBP | TTTCTGGAAAAGTTGTATTAA |
| 1403 | TBP | CTGGAAAAGTTGTATTAACAG |
| 1404 | TBP | TGGAAAAGTTGTATTAACAGG |
| 1405 | TBP | TCTTCTTAGGTGCTAAAGTCA |
| 1406 | TBP | TTAGGTGCTAAAGTCAGAGCA |
| 1407 | TBP | GGTGCTAAAGTCAGAGCAGAA |
| 1408 | TBP | TAAAGGGATTCAGGAAGACGA |
| 1409 | TBP | GGTCAAGTTTACAACCAAGAT |
| 1410 | TBP | AGGTCAAGTTTACAACCAAGA |
| 1411 | TBP | GGGCACGAAGTGCAATGGTCT |
| 1412 | TBP | CGGGCACGAAGTGCAATGGTC |
| 1413 | TBP | GGCGTTTCGGGCACGAAGTGC |
| 1414 | TBP | TATTCGGCGTTTCGGGCACGA |
| 1415 | TBP | GGATTATATTCGGCGTTTCGG |
| 1416 | TBP | AAATAGATCTAACCTTGGGAT |
| 1417 | TBP | TCCTCATGATTACCGCAGCAA |
| 1418 | TBP | GTGGCTCTCTTATCCTCATGA |
| 1419 | TBP | CCAGAACTGAAAATCAGTGCC |
| 1420 | TBP | CCCAGAACTGAAAATCAGTGC |
| 1421 | TBP | TCCCAGAACTGAAAATGAGTG |
| 1422 | TBP | GCTCCTGTGCACACCATTTTC |
| 1423 | TBP | CGGCTACCTCTTGGCTCCTGT |
| 1424 | TBP | TTACGGCTACCTCTTGGCTCC |
| 1425 | TBP | CTTACGGCTACCTCTTGGCTC |
| 1426 | TBP | CTGCCAGTCTGGACTGTTCTT |
| 1427 | TBP | TTGCTGCCAGTCTGGACTGTT |
| 1428 | TBP | CTTGCTGCCAGTCTGGACTGT |
| 1429 | TBP | TCTTGCTGCCAGTCTGGACTG |
| 1430 | TBP | TGTAGAACTCTAGCATATTTT |
| 1431 | TBP | GCTGGAAAACCCAACTTCTGT |
| 1432 | TBP | AAGTCCAAGAACTTAGCTGGA |
| 1433 | TBP | TGAATCTTGAAGTCCAAGAAC |
| 1434 | TBP | ACATCACAGCTCCCCACCATA |
| 1435 | TBP | TAACCTTATAGGAAACTTCAC |
| 1436 | TBP | GTGGGTGAGCACAAGGCCTTC |
| 1437 | TBP | TTGGTGGGTGAGCACAAGGCC |
| 1438 | TBP | CCTACTAAATTGTTGGTGGGT |
| 1439 | TBP | AGACTTAGCTAGTAAATTGTT |
| 1440 | TBP | CAGACTTAGCTAGTAAATTGT |
| 1441 | TBP | AACCAGGAAATAACTCTGGCT |
| 1442 | TBP | TGTAGATTAAACCAGGAAATA |
| 1443 | TBP | ATCATTCTGTAGATTAAACCA |
| 1444 | TBP | GATCATTCTGTAGATTAAACC |
| 1445 | TBP | TGGGTTTGATCATTCTGTAGA |
| 1446 | TBP | CAGAAACAAAAATAAGGAGAA |
| 1447 | TBP | CCAGAAACAAAAATAAGGAGA |
| 1448 | TBP | TCCAGAAACAAAAATAAGGAG |
| 1449 | TBP | ATACAACTTTTCCAGAAACAA |
| 1450 | TBP | CCTGTTAATACAACTTTTCCA |
| 1451 | TBP | CAACTTACCTGTTAATACAAC |
| 1452 | TBP | CTGTTACAACTTACCTGTTAA |
| 1453 | TBP | ATAAATTTCTGCTCTGACTTT |
| 1454 | TBP | AAATGCTTCATAAATTTCTGC |
| 1455 | TBP | CAAATGCTTCATAAATTTCTG |
| 1456 | TBP | TCAAATGCTTCATAAATTTCT |
| 1457 | TBP | CTGAATCCCTTTAGAATAGGG |
| 1458 | TBP | CGTCGTCTTCCTGAATCCCTT |
| 1459 | E2F4 | GGGGGCTATCATTGTAGTGAG |
| 1460 | E2F4 | GGGGCTATCATTGTAGTGAGT |
| 1461 | E2F4 | TAGTGAGTGGCGGCCCTGGGA |
| 1462 | E2F4 | ACTCCCACTGGGCCCAACAAC |
| 1463 | E2F4 | TGCCCTGCTGGACAGCAGCAG |
| 1464 | E2F4 | GTCCGGACCCAACCCTTCTAC |
| 1465 | E2F4 | TACCTCCTTTGAGCCCATCAA |
| 1466 | E2F4 | GAGCCCATCAAGGCAGACCCC |
| 1467 | E2F4 | AGCCCATCAAGGCAGACCCCA |
| 1468 | E2F4 | CTTGTTTTTCAGTTTTGGAAC |
| 1469 | E2F4 | TTTTTCAGTTTTGGAACTCCC |
| 1470 | E2F4 | TTCAGTTTTGGAACTCCCCAA |
| 1471 | E2F4 | TCAGTTTTGGAACTCCCCAAA |
| 1472 | E2F4 | CAGTTTTGGAACTCCCCAAAG |
| 1473 | E2F4 | AGTTTTGGAACTCCCCAAAGA |
| 1474 | E2F4 | TGGAACTCCCCAAAGAGCTGT |
| 1475 | E2F4 | GGAACTCCCCAAAGAGCTGTC |
| 1476 | E2F4 | CCAGAGTGCATGAGCTCGGAG |
| 1477 | E2F4 | GCCCCTCTGCTTCGTCTTTCT |
| 1478 | E2F4 | CCCCTCTGCTTCGTCTTTCTC |
| 1479 | E2F4 | GTCTTTCTCCACCCCCGGGAG |
| 1480 | E2F4 | CTCCACCCCCGGGAGACCACG |
| 1481 | E2F4 | TCCACCCCCGGGAGACCACGA |
| 1482 | E2F4 | TATCTACAACCTGGAGGAGAG |
| 1483 | E2F4 | GATGTGCCTGTTCTCAACCTC |
| 1484 | E2F4 | ATGTGCCTGTTCTCAACCTCT |
| 1485 | E2F4 | TGCACTGCCAGGGACAGCAGT |
| 1486 | E2F4 | CCTGGACTTCTGCACTGCCAG |
| 1487 | E2F4 | CTATCAGTCCCAGGGCCGCCA |
| 1488 | E2F4 | GGCCCAGTGGGAGTGAACTGA |
| 1489 | E2F4 | TTGGGCCCAGTGGGAGTGAAC |
| 1490 | E2F4 | GGTCCGGACGAACTGCTGCTG |
| 1491 | E2F4 | ATGGGCTCAAAGGAGGTAGAA |
| 1492 | E2F4 | TGACAGCTCTTTGGGGAGTTC |
| 1493 | E2F4 | CTGACAGCTCTTTGGGGAGTT |
| 1494 | E2F4 | TGAGGACATCAACTCCTCCAG |
| 1495 | E2F4 | CAGGGCCACCCACCTTCTGAG |
| 1496 | E2F4 | TAGATATAATCGTGGTCTCCC |
| 1497 | E2F4 | ACTCTCGTCCAGGTTGTAGAT |
| 1498 | G6PD | TGGGGGTTCACCCACTTGTAG |
| 1499 | G6PD | ACCCACTTGTAGGTGCCCTCA |
| 1500 | G6PD | TAGGTGCCCTCATACTGGAAA |
| 1501 | G6PD | ATCAGCTCGTCTGCCTCCGTG |
| 1502 | G6PD | CCTCACCTGCCATAAATATAG |
| 1503 | G6PD | CTCACCTGCCATAAATATAGG |
| 1504 | G6PD | GGCTTCTCCAGCTCAATCTGG |
| 1505 | G6PD | TCCAGCTCAATCTGGTGCAGC |
| 1506 | G6PD | TCTGTAGGGCACCTTGTATCT |
| 1507 | G6PD | TATCTGTTGCCGTAGGTCAGG |
| 1508 | G6PD | CCGTAGGTCAGGTCCAGCTCC |
| 1509 | G6PD | AAGAACATGCCCGGCTTCTTG |
| 1510 | G6PD | TTGGTCATCATCTTGGTGTAC |
| 1511 | G6PD | GTCATCATCTTGGTGTACACG |
| 1512 | G6PD | GTGTACACGGCCTCGTTGGGC |
| 1513 | G6PD | GGCTGCACGCGGATCACCAGC |
| 1514 | G6PD | CGCTTGCACTGCTGGTGGAAG |
| 1515 | G6PD | CACTGCTGGTGGAAGATGTCG |
| 1516 | G6PD | CGCTCGTTCAGGGCCTTGCCG |
| 1517 | G6PD | AGGGCCTTGCCGCAGCGCAGG |
| 1518 | G6PD | CCGCAGCGCAGGATGAAGGGC |
| 1519 | G6PD | CAGTATGAGGGCACCTACAAG |
| 1520 | G6PD | CCAGTATGAGGGCACCTACAA |
| 1521 | G6PD | AGCTGGAGAAGCCCAAGCCCA |
| 1522 | G6PD | ACCCCACTGCTGCACCAGATT |
| 1523 | G6PD | CACCCCACTGCTGCACCAGAT |
| 1524 | G6PD | TCACCCCACTGCTGCACCAGA |
| 1525 | G6PD | TGCGGGAGCCAGATGCACTTC |
| 1526 | G6PD | AACCCCGAGGAGTCGGAGCTG |
| 1527 | G6PD | TTCAACCCCGAGGAGTCGGAG |
| 1528 | G6PD | CACCAGCAGTGCAAGCGCAAC |
| 1529 | G6PD | CATGATGTGGCCGGCGACATC |
| 1530 | G6PD | ATCCTGCGCTGCGGCAAGGCC |
| 1531 | G6PD | CGCCACGTAGGGGTGCCCTTC |
| 1532 | G6PD | CCGCCACGTAGGGGTGCCCTT |
| 1533 | KIF11 | ATGAAGATAAATTGATAGCAC |
| 1534 | KIF11 | ATAGCACAAAATCTAGAACTT |
| 1535 | KIF11 | ATGAAACCATAAAAATTGGTT |
| 1536 | KIF11 | GTTTGACTAAGCTTAATTGCT |
| 1537 | KIF11 | GACTAAGCTTAATTGCTTTCT |
| 1538 | KIF11 | ACTAAGCTTAATTGCTTTCTG |
| 1539 | KIF11 | ATTGCTTTCTGGAACAGGATC |
| 1540 | KIF11 | CTTTCTGGAACAGGATCTGAA |
| 1541 | KIF11 | CTGGAACAGGATCTGAAACTG |
| 1542 | KIF11 | TGGAACAGGATCTGAAACTGG |
| 1543 | KIF11 | TCTAATGTCCGTTAAAGGTAC |
| 1544 | KIF11 | AAGGTACGACACCACAGAGGA |
| 1545 | KIF11 | TTTATACCCATCAACACTGGT |
| 1546 | KIF11 | ATACCCATCAACACTGGTAAG |
| 1547 | KIF11 | TACCCATCAACACTGGTAAGA |
| 1548 | KIF11 | ATCAGCTGAAAAGGAAACAGC |
| 1549 | KIF11 | ATGATGCTAAACTGTTCAGAA |
| 1550 | KIF11 | AGAAAACAACAAAGAAGAGAC |
| 1551 | KIF11 | CTTCTTTTAGGATGTGGATGT |
| 1552 | KIF11 | TTCTTTTAGGATGTGGATGTA |
| 1553 | KIF11 | TTTTAGGATGTGGATGTAGAA |
| 1554 | KIF11 | TAGGATGTGGATGTAGAAGAG |
| 1555 | KIF11 | AGGATGTGGATGTAGAAGAGG |
| 1556 | KIF11 | GGATGTGGATGTAGAAGAGGC |
| 1557 | KIF11 | TGGGGCAGTATACTGAAGAAC |
| 1558 | KIF11 | TTCATCAATTGGCGGGGTTCC |
| 1559 | KIF11 | ATCAATTGGCGGGGTTCCATT |
| 1560 | KIF11 | GCGGGGTTCCATTTTTCCAGG |
| 1561 | KIF11 | TCCCGCCTTAAATCCACAGCA |
| 1562 | KIF11 | CCCGCCTTAAATCCACAGCAT |
| 1563 | KIF11 | CCGCCTTAAATCCACAGCATA |
| 1564 | KIF11 | AATCCACAGCATAAAAAATCA |
| 1565 | KIF11 | ACACACTGGAGAGGTCTAAAG |
| 1566 | KIF11 | GTTACAAAGAGCAGATTACCT |
| 1567 | KIF11 | CAAAGAGCAGATTACCTCTGC |
| 1568 | KIF11 | CCTCTGCGAGCCCAGATCAAC |
| 1569 | KIF11 | TAGATTTTGTGCTATCAATTT |
| 1570 | KIF11 | AGTTCTAGATTTTGTGCTATC |
| 1571 | KIF11 | ATTAAGTTCTAGATTTTGTGC |
| 1572 | KIF11 | CATTAAGTTCTAGATTTTGTG |
| 1573 | KIF11 | TGGTTTCATTAAGTTCTAGAT |
| 1574 | KIF11 | ATGGTTTCATTAAGTTCTAGA |
| 1575 | KIF11 | TATGGTTTCATTAAGTTCTAG |
| 1576 | KIF11 | TTATGGTTTCATTAAGTTCTA |
| 1577 | KIF11 | GTCAAACCAATTTTTATGGTT |
| 1578 | KIF11 | AGCTTAGTCAAACCAATTTTT |
| 1579 | KIF11 | CAGAAAGCAATTAAGCTTAGT |
| 1580 | KIF11 | AGATCCTGTTCCAGAAAGCAA |
| 1581 | KIF11 | CAGATCCTGTTCCAGAAAGCA |
| 1582 | KIF11 | GGATATCCAGTTTCAGATCCT |
| 1583 | KIF11 | AAGTACCTGTTGGGATATCCA |
| 1584 | KIF11 | AAAGTACCTGTTGGGATATCC |
| 1585 | KIF11 | TAAAGTACCTGTTGGGATATC |
| 1586 | KIF11 | TCTTTTAAAGTACCTGTTGGG |
| 1587 | KIF11 | CTCTTTTAAAGTACCTGTTGG |
| 1588 | KIF11 | TATTTCTCTTTTAAAGTACCT |
| 1589 | KIF11 | ATGGGTATAAATAACTTTTCC |
| 1590 | KIF11 | CCAGTGTTGATGGGTATAAAT |
| 1591 | KIF11 | TTACCAGTGTTGATGGGTATA |
| 1592 | KIF11 | AGTTCTTACCAGTGTTGATGG |
| 1593 | KIF11 | ACGTGGTTCAGTTCTTACCAG |
| 1594 | KIF11 | AGCTGATCAAGGAGATGTTCA |
| 1595 | KIF11 | CAGCTGATCAAGGAGATGTTC |
| 1596 | KIF11 | TCAGCTGATCAAGGAGATGTT |
| 1597 | KIF11 | CTTTTCAGCTGATCAAGGAGA |
| 1598 | KIF11 | CCTTTTCAGCTGATCAAGGAG |
| 1599 | KIF11 | ACAGCTCAGGCTGTTTCCTTT |
| 1600 | KIF11 | GCATCATTAACAGCTCAGGCT |
| 1601 | KIF11 | AGCATCATTAACAGCTCAGGC |
| 1602 | KIF11 | TGAACAGTTTAGCATCATTAA |
| 1603 | KIF11 | CTGAACAGTTTAGCATCATTA |
| 1604 | KIF11 | TCTGAACAGTTTAGCATCATT |
| 1605 | KIF11 | TTTTCTGAACAGTTTAGCATC |
| 1606 | KIF11 | TTGTTTTCTGAACAGTTTAGC |
| 1607 | KIF11 | TTTGTTGTTTTCTGAACAGTT |
| 1608 | KIF11 | TCTCTTCTTTGTTGTTTTCTG |
| 1609 | KIF11 | CCGGAATTGTCTCTTCTTTGT |
| 1610 | KIF11 | ACCGGAATTGTCTCTTCTTTG |
| 1611 | KIF11 | AATTTACCGGAATTGTCTCTT |
| 1612 | KIF11 | AAATTTACCGGAATTGTCTCT |
| 1613 | KIF11 | AGTATACTGCCCCAGAACTGC |
| 1614 | KIF11 | TTCAGTATACTGCCCCAGAAC |
| 1615 | KIF11 | GAGGTTCTTCAGTATACTGCC |
| 1616 | KIF11 | ACTTAGAGGTTCTTCAGTATA |
| 1617 | KIF11 | ATGAACAATCCACACCAGCAT |
| 1618 | KIF11 | TCTGATATGACATACCTGGAA |
| 1619 | KIF11 | TCTTTTCCATGTGATTTTTTA |
| 1620 | KIF11 | GTCTTTTCCATGTGATTTTTT |
| 1621 | KIF11 | TTTGTCTTTTCCATGTGATTT |
| 1622 | KIF11 | CTTTGTCTTTTCCATGTGATT |
| 1623 | KIF11 | TCTTTGTCTTTTCCATGTGAT |
| 1624 | KIF11 | ATGCCTCTGTTTTCTTTGTCT |
| 1625 | KIF11 | GACCTCTCCAGTGTGTTAATG |
| 1626 | KIF11 | AGACCTCTCCAGTGTGTTAAT |
| 1627 | KIF11 | CACTTTAGACCTCTCCAGTGT |
| 1628 | KIF11 | TTCCACTTTAGACCTCTCCAG |
| 1629 | KIF11 | CTTCCACTTTAGACCTCTCCA |
| 1630 | KIF11 | TAACCAAGTGCTCTGTAGTTT |
| 1631 | KIF11 | GTAACCAAGTGCTCTGTAGTT |
| 1632 | KIF11 | ATCTGGGCTCGCAGAGGTAAT |
| 1633 | KIF11 | AAGGTTGATCTGGGCTCGCAG |
| 1634 | KIF11 | CCAACCCCCAAGTGAATTAAA |
| — | — | — |

TABLE 22
SpyCas9 guide RNAs

| SEQ | | Target Domain |
|---|---|---|
| ID NO | Gene | Sequence (DNA) |
| 1635 | GAPDH | TCTAGGTATGAGAACGAATT |
| 1636 | GAPDH | AGCCCCAGCGTCAAAGGTGG |
| 1637 | TBP | ATTGTATCCACAGTGAATCT |
| 1638 | TBP | AAACGCCGAATATAATCCCA |
| 1639 | TBP | ACCATTGTAGCGGTTTGCTG |
| 1640 | TBP | GGTTTGCTGCGGTAATCATG |
| 1641 | TBP | GATAAGAGAGCCACGAACCA |
| 1642 | TBP | ACGGCACTGATTTTCAGTTC |
| 1643 | TBP | CGGCACTGATTTTCAGTTCT |
| 1644 | TBP | GATTTTCAGTTCTGGGAAAA |
| 1645 | TBP | TCTGGGAAAATGGTGTGCAC |
| 1646 | TBP | TGGTGTGCACAGGAGCCAAG |
| 1647 | TBP | TAGTGAAGAACAGTCCAGAC |
| 1648 | TBP | TGCTAGAGTTGTACAGAAGT |
| 1649 | TBP | GCTAGAGTTGTACAGAAGTT |
| 1650 | TBP | GGGTTTTCCAGCTAAGTTCT |
| 1651 | TBP | GGACTTCAAGATTCAGAATA |
| 1652 | TBP | CTTCAAGATTCAGAATATGG |
| 1653 | TBP | TTCAAGATTCAGAATATGGT |
| 1654 | TBP | TCAAGATTCAGAATATGGTG |
| 1655 | TBP | GTGATGTGAAGTTTCCTATA |
| 1656 | TBP | AAGTTTCCTATAAGGTTAGA |
| 1657 | TBP | TCACCCACCAACAATTTAGT |
| 1658 | TBP | TATGAGCCAGAGTTATTTCC |
| 1659 | TBP | GTTCTCCTTATTTTTGTTTC |
| 1660 | TBP | TCTGGAAAAGTTGTATTAAC |
| 1661 | TBP | AAACATCTACCCTATTCTAA |
| 1662 | TBP | ACCCTATTCTAAAGGGATTC |
| 1663 | TBP | GATTCAGGAAGACGACGTAA |
| 1664 | TBP | CACGAAGTGCAATGGTCTTT |
| 1665 | TBP | GTTTCGGGCACGAAGTGCAA |
| 1666 | TBP | GGGATTATATTCGGCGTTTC |
| 1667 | TBP | TGGGATTATATTCGGCGTTT |
| 1668 | TBP | TCTAACCTTGGGATTATATT |
| 1669 | TBP | ATTAAAATAGATCTAACCTT |
| 1670 | TBP | AAAATCAGTGCCGTGGTTCG |
| 1671 | TBP | AGAACTGAAAATCAGTGCCG |
| 1672 | TBP | AATTTCTTACGGCTACCTCT |
| 1673 | TBP | AGTCTGGACTGTTCTTCACT |
| 1674 | TBP | ATATTTTCTTGCTGCCAGTC |
| 1675 | TBP | TTGAAGTCCAAGAACTTAGC |
| 1676 | TBP | ACAAGGCCTTCTAACCTTAT |
| 1677 | TBP | ATTGTTGGTGGGTGAGCACA |
| 1678 | TBP | TTACCTACTAAATTGTTGGT |
| 1679 | TBP | CTTACCTACTAAATTGTTGG |
| 1680 | TBP | AGACTTAGCTAGTAAATTGT |
| 1681 | TBP | ATTAAACCAGGAAATAACTC |
| 1682 | TBP | ATCATTCTGTAGATTAAACC |
| 1683 | TBP | AAAATAAGGAGAACAATTCT |
| 1684 | TBP | CTTTTCCAGAAACAAAAATA |
| 1685 | TBP | TCCTGAATCCCTTTAGAATA |
| 1686 | TBP | TTCCTGAATCCCTTTAGAAT |
| 1687 | E2F4 | CTCACTCCCACTGCTGTCCC |
| 1688 | E2F4 | CCCTGGCAGTGCAGAAGTCC |
| 1689 | E2F4 | CCTGGCAGTGCAGAAGTCCA |
| 1690 | E2F4 | CAGTGCAGAAGTCCAGGGAA |
| 1691 | E2F4 | GCAGAAGTCCAGGGAATGGC |
| 1692 | E2F4 | GGCCCAGCAGCTGAGATCAC |
| 1693 | E2F4 | GGGGCTATCATTGTAGTGAG |
| 1694 | E2F4 | GCTATCATTGTAGTGAGTGG |
| 1695 | E2F4 | ATTGTAGTGAGTGGCGGCCC |
| 1696 | E2F4 | TTGTAGTGAGTGGCGGCCCT |
| 1697 | E2F4 | CGGCCCTGGGACTGATAGCA |
| 1698 | E2F4 | GGGACTGATAGCAAGGACAG |
| 1699 | E2F4 | TGAGCTCAGTTCACTCCCAC |
| 1700 | E2F4 | GAGCTCAGTTCACTCCCACT |
| 1701 | E2F4 | CCCACTGGGCCCAACAACAC |
| 1702 | E2F4 | GCCCAACAACACTGGACACC |
| 1703 | E2F4 | ACTGCAGTCTTCTGCCCTGC |
| 1704 | E2F4 | AGTAACAGCAGCAGTTCGTC |
| 1705 | E2F4 | TACCTCCTTTGAGCCCATCA |
| 1706 | E2F4 | CCCATCAAGGCAGACCCCAC |
| 1707 | E2F4 | ATCAAGGCAGACCCCACAGG |
| 1708 | E2F4 | GAAATCTTTGATCCCACACG |
| 1709 | E2F4 | TCTTTGATCCCACACGAGGT |
| 1710 | E2F4 | ATTCCCAGAGTGCATGAGCT |
| 1711 | E2F4 | GTGCATGAGCTCGGAGCTGC |
| 1712 | E2F4 | GAGGAGTTGATGTCCTCAGA |
| 1713 | E2F4 | GAGTTGATGTCCTCAGAAGG |
| 1714 | E2F4 | AGTTGATGTCCTCAGAAGGT |
| 1715 | E2F4 | GCTTCGTCTTTCTCCACCCC |
| 1716 | E2F4 | CTTCGTCTTTCTCCACCCCC |
| 1717 | E2F4 | CCACGATTATATCTACAACC |
| 1718 | E2F4 | TACAACCTGGACGAGAGTGA |
| 1719 | E2F4 | GCACTGCCAGGGACAGCAGT |
| 1720 | E2F4 | TGCACTGCCAGGGACAGCAG |
| 1721 | E2F4 | CCTGGACTTCTGCACTGCCA |
| 1722 | E2F4 | CCCTGGACTTCTGCACTGCC |
| 1723 | E2F4 | CTGCTGGGCCAGCCATTCCC |
| 1724 | E2F4 | TGTCCTTGCTATCAGTCCCA |
| 1725 | E2F4 | CTGTCCTTGCTATCAGTCCC |
| 1726 | E2F4 | CCAGTGTTGTTGGGCCCAGT |
| 1727 | E2F4 | TCCAGTGTTGTTGGGCCCAG |
| 1728 | E2F4 | GCCGGGTGTCCAGTGTTGTT |
| 1729 | E2F4 | GGCCGGGTGTCCAGTGTTGT |
| 1730 | E2F4 | AGCAGGGCAGAAGACTGCAG |
| 1731 | E2F4 | GCTGCTGCTGCTGTCCAGCA |
| 1732 | E2F4 | GGAGGTAGAAGGGTTGGGTC |
| 1733 | E2F4 | TGGGCTCAAAGGAGGTAGAA |
| 1734 | E2F4 | ATGGGCTCAAAGGAGGTAGA |
| 1735 | E2F4 | TGCCTTGATGGGCTCAAAGG |
| 1736 | E2F4 | GTCTGCCTTGATGGGCTCAA |
| 1737 | E2F4 | CCTGTGGGGTCTGCCTTGAT |
| 1738 | E2F4 | ACCTGTGGGGTCTGCCTTGA |
| 1739 | E2F4 | GCAGGTACTCACCACCTGTG |
| 1740 | E2F4 | GGCAGGTACTCACCACCTGT |
| 1741 | E2F4 | GGGCAGGTACTCACCACCTG |
| 1742 | E2F4 | AGATTTCTGACAGCTCTTTG |
| 1743 | E2F4 | AAGATTTCTGACAGCTCTTT |
| 1744 | E2F4 | AAAGATTTCTGACAGCTCTT |
| 1745 | E2F4 | TGCAGCAGCCTACCTCGTGT |
| 1746 | E2F4 | ATGCAGCAGCCTACCTCGTG |
| 1747 | E2F4 | GCTCCGAGCTCATGCACTCT |
| 1748 | E2F4 | AGCTCCGAGCTCATGCACTC |
| 1749 | E2F4 | CCAGGGCCACCCACCTTCTG |
| 1750 | E2F4 | TGGAGAAAGACGAAGCAGAG |
| 1751 | E2F4 | GTGGAGAAAGACGAAGCAGA |
| 1752 | E2F4 | GGTGGAGAAAGACGAAGCAG |
| 1753 | E2F4 | TAATCGTGGTCTCCCGGGGG |
| 1754 | E2F4 | ATATAATCGTGGTCTCCCGG |
| 1755 | E2F4 | GATATAATCGTGGTCTCCCG |
| 1756 | E2F4 | AGATATAATCGTGGTCTCCC |
| 1757 | E2F4 | TAGATATAATCGTGGTCTCC |
| 1758 | E2F4 | CGAGGTTGTAGATATAATCG |
| 1759 | E2F4 | AGACACCTTCACTCTCGTCC |
| 1760 | E2F4 | TGAGAACAGGCACATCAAAG |
| 1761 | G6PD | GTGGGGGTTCACCCACTTGT |
| 1762 | G6PD | ACTTGTAGGTGCCCTCATAC |
| 1763 | G6PD | CATCAGCTCGTCTGCCTCCG |
| 1764 | G6PD | ATCAGCTCGTCTGCCTCCGT |
| 1765 | G6PD | TCAGCTCGTCTGCCTCCGTG |
| 1766 | G6PD | CGTCTGCCTCCGTGGGGCCT |
| 1767 | G6PD | TGCCTCCGTGGGGCCTCGGC |
| 1768 | G6PD | TCCTCACCTGCCATAAATAT |
| 1769 | G6PD | CCTCACCTGCCATAAATATA |
| 1770 | G6PD | CTCACCTGCCATAAATATAG |
| 1771 | G6PD | CCTGCCATAAATATAGGGGA |
| 1772 | G6PD | CTGCCATAAATATAGGGGAT |
| 1773 | G6PD | ATAAATATAGGGGATGGGCT |
| 1774 | G6PD | TAAATATAGGGGATGGGCTT |
| 1775 | G6PD | TGGGCTTCTCCAGCTCAATC |
| 1776 | G6PD | AGCTCAATCTGGTGCAGCAG |
| 1777 | G6PD | GCTCAATCTGGTGCAGCAGT |
| 1778 | G6PD | CTCAATCTGGTGCAGCAGTG |
| 1779 | G6PD | CAGTGGGGTGAAAATACGCC |
| 1780 | G6PD | TGAAAATACGCCAGGCCTCA |
| 1781 | G6PD | CCTCACGGAGCTCGTCGCTG |
| 1782 | G6PD | ACCTGCGCACGAAGTGCATC |
| 1783 | G6PD | GGCTCCCGCAGAAGACGTCC |
| 1784 | G6PD | CGCAGAAGACGTCCAGGATG |
| 1785 | G6PD | GTCCAGGATGAGGCGCTCAT |
| 1786 | G6PD | ATGAGGCGCTCATAGGCGTC |
| 1787 | G6PD | TGAGGCGCTCATAGGCGTCA |
| 1788 | G6PD | CACCTTGTATCTGTTGCCGT |
| 1789 | G6PD | TGTATCTGTTGCCGTAGGTC |
| 1790 | G6PD | CAGGTCCAGCTCCGACTCCT |
| 1791 | G6PD | AGGTCCAGCTCCGACTCCTC |
| 1792 | G6PD | GGTCCAGCTCCGACTCCTCG |
| 1793 | G6PD | TCGGGGTTGAAGAACATGCC |
| 1794 | G6PD | GAAGAACATGCCCGGCTTCT |
| 1795 | G6PD | CGGCTTCTTGGTCATCATCT |
| 1796 | G6PD | GGTCATCATCTTGGTGTACA |
| 1797 | G6PD | CTTGGTGTACACGGCCTCGT |
| 1798 | G6PD | TTGGTGTACACGGCCTCGTT |
| 1799 | G6PD | CGGCCTCGTTGGGCTGCACG |
| 1800 | G6PD | GCTCGTTGCGCTTGCACTGC |
| 1801 | G6PD | CGTTGCGCTTGCACTGCTGG |
| 1802 | G6PD | CTGCTGGTGGAAGATGTCGC |
| 1803 | G6PD | AGATGTCGCCGGCCACATCA |
| 1804 | G6PD | ATGGAACTGCAGCCTCACCT |
| 1805 | G6PD | CCTCGGCCTTGCGCTCGTTC |
| 1806 | G6PD | CTCGGCCTTGCGCTCGTTCA |
| 1807 | G6PD | TCAGGGCCTTGCCGCAGCGC |
| 1808 | G6PD | CTTGCCGCAGCGCAGGATGA |
| 1809 | G6PD | TTGCCGCAGCGCAGGATGAA |
| 1810 | G6PD | GTATGAGGGCACCTACAAGT |
| 1811 | G6PD | AGTATGAGGGCACCTACAAG |
| 1812 | G6PD | AGAGTGGGTTTCCAGTATGA |
| 1813 | G6PD | GAGAGTGGGTTTCCAGTATG |
| 1814 | G6PD | GACGAGCTGATGAAGAGAGT |
| 1815 | G6PD | AGACGAGCTGATGAAGAGAG |
| 1816 | G6PD | CTCCAGCCGAGGCCCCACGG |
| 1817 | G6PD | CACCCGTCACTCTCCAGCCG |
| 1818 | G6PD | CCATCCCCTATATTTATGGC |
| 1819 | G6PD | AAGCCCATCCCCTATATTTA |
| 1820 | G6PD | ACTGCTGCACCAGATTGAGC |
| 1821 | G6PD | GCGACGAGCTCCGTGAGGCC |
| 1822 | G6PD | CCTCAGCGACGAGCTCCGTG |
| 1823 | G6PD | GCCAGATGCACTTCGTGCGC |
| 1824 | G6PD | TCATCCTGGACGTCTTCTGC |
| 1825 | G6PD | CTCATCCTGGACGTCTTCTG |
| 1826 | G6PD | CGCCTATGAGCGCCTCATCC |
| 1827 | G6PD | GACCTACGGCAACAGATACA |
| 1828 | G6PD | TCGGAGCTGGACCTGACCTA |
| 1829 | G6PD | CAACCCCGAGGAGTCGGAGC |
| 1830 | G6PD | GTTCTTCAACCCCGAGGAGT |
| 1831 | G6PD | GGGCATGTTCTTCAACCCCG |
| 1832 | G6PD | AAGATGATGACCAAGAAGCC |
| 1833 | G6PD | CAAGATGATGACCAAGAAGC |
| 1834 | G6PD | GATCCGCGTGCAGCCCAACG |
| 1835 | G6PD | GCAGTGCAAGCGCAACGAGC |
| 1836 | G6PD | CTGCAGTTCCATGATGTGGC |
| 1837 | G6PD | GAGGCTGCAGTTCCATGATG |
| 1838 | G6PD | ACGAGCGCAAGGCCGAGGTG |
| 1839 | G6PD | CCTGAACGAGCGCAAGGCCG |
| 1840 | G6PD | CAAGGCCCTGAACGAGCGCA |
| 1841 | G6PD | CTTCATCCTGCGCTGCGGCA |
| 1842 | G6PD | GTGCCCTTCATCCTGCGCTG |
| 1843 | G6PD | AGAATGAGAGGTGGGATGGT |
| 1844 | G6PD | GTGGAGAATGAGAGGTGGGA |
| 1845 | KIF11 | CTTAATGAAACCATAAAAAT |
| 1846 | KIF11 | GACTAAGCTTAATTGCTTTC |
| 1847 | KIF11 | GCTTAATTGCTTTCTGGAAC |
| 1848 | KIF11 | TCTGGAACAGGATCTGAAAC |
| 1849 | KIF11 | CTGAAACTGGATATCCCAAC |
| 1850 | KIF11 | TTAAAGGTACGACACCACAG |
| 1851 | KIF11 | TTATTTATACCCATCAACAC |
| 1852 | KIF11 | ATCTCCTTGATCAGCTGAAA |
| 1853 | KIF11 | CAACAAAGAAGAGACAATTC |
| 1854 | KIF11 | TTAGGATGTGGATGTAGAAG |
| 1855 | KIF11 | GGATGTAGAAGAGGCAGTTC |
| 1856 | KIF11 | GATGTAGAAGAGGCAGTTCT |
| 1857 | KIF11 | ATGTAGAAGAGGCAGTTCTG |
| 1858 | KIF11 | CAAGAGCCATCTGTAGATGC |
| 1859 | KIF11 | GCCATCTGTAGATGCTGGTG |
| 1860 | KIF11 | GGTGTGGATTGTTCATCAAT |
| 1861 | KIF11 | GTGGATTGTTCATCAATTGG |
| 1862 | KIF11 | TGGATTGTTCATCAATTGGC |
| 1863 | KIF11 | GGATTGTTCATCAATTGGCG |
| 1864 | KIF11 | TGGCGGGGTTCCATTTTTCC |
| 1865 | KIF11 | CCACAGCATAAAAAATCACA |
| 1866 | KIF11 | GGAAAAGACAAAGAAAACAG |
| 1867 | KIF11 | AAACAGAGGCATTAACACAC |
| 1868 | KIF11 | GAGGCATTAACACACTGGAG |
| 1869 | KIF11 | CACACTGGAGAGGTCTAAAG |
| 1870 | KIF11 | GGAAGAAACTACAGAGCACT |
| 1871 | KIF11 | CTTAGTCAAAGCAATTTTTA |
| 1872 | KIF11 | TCTCTTTTAAAGTACCTGTT |
| 1873 | KIF11 | TTCTCTTTTAAAGTACCTGT |
| 1874 | KIF11 | TATAAATAACTTTTCCTCTG |
| 1875 | KIF11 | CAGTTCTTACCAGTGTTGAT |
| 1876 | KIF11 | TCAGTTCTTACCAGTGTTGA |
| 1877 | KIF11 | TGATCAAGGAGATGTTCACG |
| 1878 | KIF11 | GTTTCCTTTTCAGCTGATCA |
| 1879 | KIF11 | TTTAGCATCATTAACAGCTC |
| 1880 | KIF11 | ACAGATGGCTCTTGACTTAG |
| 1881 | KIF11 | TCCACACCAGCATCTAGAGA |
| 1882 | KIF11 | ATATGACATACCTGGAAAAA |
| 1883 | KIF11 | AGGTTGATCTGGGCTCGCAG |
| 1884 | KIF11 | AGTGAATTAAAGGTTGATCT |
| 1885 | KIF11 | AAGTGAATTAAAGGTTGATC |
| — | — | — |

### Example 20—a Second Round of Editing with RNP and Donor Templates or RNP Alone Enables Further Enrichment of iPSCs with Transgenes Targeted at the GAPDH Gene Locus

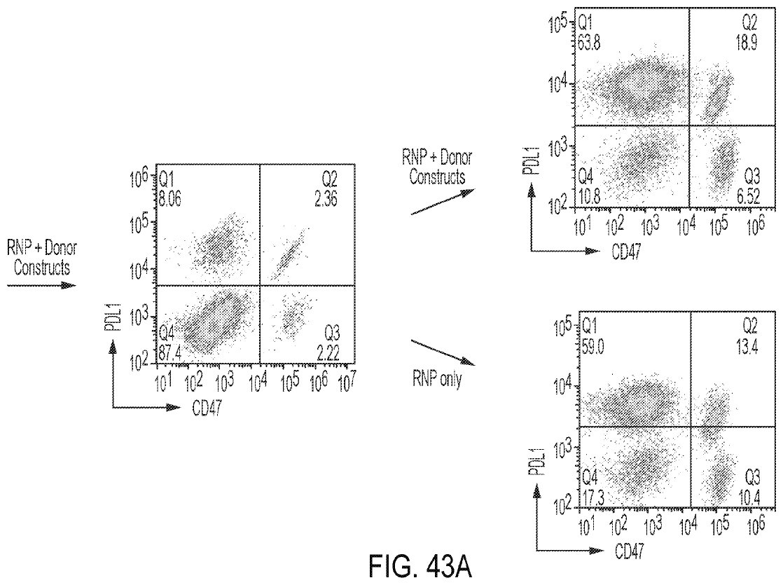

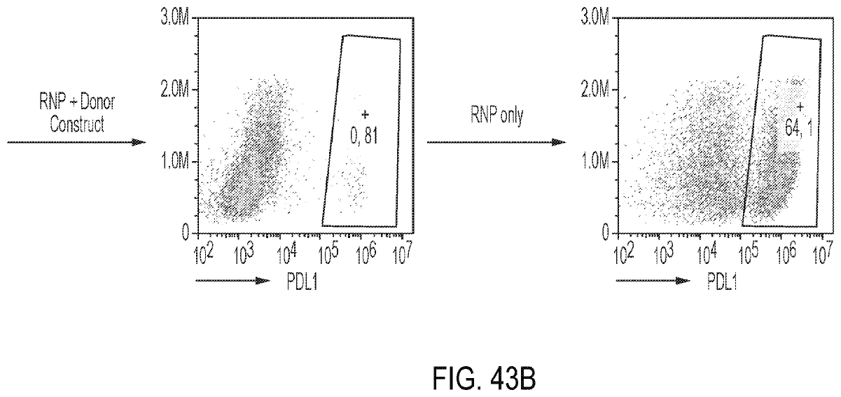

The present example relates to the introduction of two immunologically relevant genes inserted biallelically, and in a bicistronic manner at the GAPDH gene. Two different donor templates (e.g., donor nucleic acid constructs) one containing the gene sequences for the PDL1 immuno-regulatory molecule and a safety switch as its genetic payload, and the other donor template comprising CD47 immuno-regulatory molecule and the same safety switch as its genetic payload were targeted to the GAPDH locus (FIG. 43A). Following the first round of editing with ribonucleoprotein (RNP) Cpf1 nuclease and guide RNA complex gene editing system, PDL1-based and CD47-based donor templates, ˜8.1% of PDL1-positive, ˜2.2% of CD47-positive, and ˜2.4% of PDL1/CD47-double positive cells were obtained. This indicated that donor nucleic acid constructs with their flanking homology arms had integrated correctly at the GAPDH locus restoring the disruption that had been caused by nuclease cutting within the GAPDH exon. This result was surprising because the double positive results were far superior to expected and previously seen results (e.g., as described in the art). Note that the single knock-in efficiency for CD47 was lower than the double knock in, potentially because PD-L1 incorporation was more efficient and assisted with higher rates of biallelic incorporation.

To further enrich for the population of edited cells, cells were expanded and then re-edited by providing the pool of surviving cells with either RNP and both donor templates (e.g., donor nucleic acid constructs) again, or RNP alone. In the sample re-edited with RNP and both donor templates (e.g., donor nucleic acid constructs), the population of PDL1-positive cells increased to ˜63.8%, the population of CD47-positive cells increased to ˜6.5%, and the population of PDL1/CD47-double positive cells increased to ˜18.9%. In the sample re-edited with RNP only, the population of PDL1-positive cells increased to ˜59.0%, the population of CD47-positive cells increased to ˜10.4%, and the population of PDL1/CD47-double positive cells increased to ˜13.4%. There was a decrease of unedited cells from 87.4% to 10.8% with RNP and donor templates, or to 17.3% with RNP alone. In either case, providing a second round of RNP allowed selective removal of non-targeted cells via GAPDH exon cutting, and therefore further enrichment of cells targeted with either or both of the PDL1-based and CD47-based donor templates (e.g., nucleic acid constructs).

In a separate study, the same PDL1-based donor template was used to target PDL1 to the GAPDH locus (FIG. 43B). Following the first round of editing with RNP and the PDL1-based donor template ˜0.8% of PDL1-positive cells were obtained. To further enrich for the population of edited cells, cells were expanded and then re-edited by providing the surviving population of cells with RNP alone. In the sample re-edited with RNP only, the population of PDL1-positive cells increased to 64.7%. This data indicates that editing with a second round of RNP allowed selective removal of non-targeted cells via GAPDH exon cutting, and therefore further enrichment of cells targeted with the PDL1-based donor template.

### Example 21—Editing in PSCs with Two Different Donor Templates Including Suicide Switch Components, and RNP Targeted to the Coding Region of GAPDH Gene Enables Enrichment of Biallelically Edited Cells and Therefore Dimerization of Suicide Switch Components

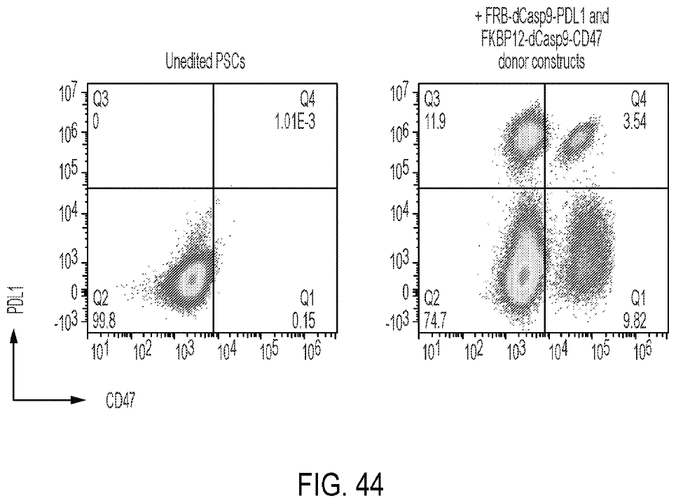

The present example relates to the introduction of two knock-in cassettes each encoding multiple gene products of interest as their genetic payloads. Two different donor templates (e.g., nucleic acid constructs) that contain the PDL1 or CD47 immuno-regulatory molecules were targeted to the GAPDH gene (FIG. 44). The PDL1-based donor template was comprised of the coding sequence for FRB (FKBP12-rapamycin binding domain fragment of, mammalian target of rapamycin(mTOR)) linked via a GS linker to the coding sequence for the truncated caspase 9 gene (dCasp9), which was linked via a P2A self-cleaving peptide to the coding sequence for the PDL1 gene. The CD47-based donor template was comprised of the coding sequence for FKBP12 (Peptidyl-prolyl cis-trans isomerase FKBP12, encoding the 12-kDa FK506-binding protein) linked via a GS linker to the coding sequence for the truncated caspase 9 gene (dCasp9), which was linked via a P2A self-cleaving peptide to the coding sequence for the PDL1 gene. The FRB-dCasp9 and FKBP12-dCasp9 sequences form the two necessary components of the rapamycin inducible Caspase 9 kill switch (rapaCasp9). In the presence of rapamycin, the FRB and FKBP12 domains will heterodimerize causing the truncated Caspase 9 proteins to homodimerize, this in turn activates downstream effector caspases to trigger apoptosis in biallelically edited rapaCasp9 cells.

After editing of PSCs with GAPDH-targeting RNP and PDL1-based and CD47-based donor templates, surviving cells were allowed to recover and expand, and flow cytometric analysis was performed one week later on a population of PSCs stained with anti-PDL-1 and anti-CD47 antibodies. After cytometric analysis, provision of surviving cells with GAPDH-targeting RNP and two different donor templates (e.g., donor nucleic acid constructs) together (FRB-dCasp9-PDL1 and FKBP12-dCasp9-CD47) resulted in PDL1-positive PSCs (˜11.9%), CD47-positive PSCs (˜9.8%), and cells that were double-positive for PDL1 and CD47 (˜3.5%), indicating that some cells had biallelically integrated the two genetic payloads: both an FRB-dCasp9-PDL1 transgene and an FKBP12-dCasp9-CD47 transgene targeted to the GAPDH gene, which restored the disruption that had been caused by nuclease cutting within the GAPDH exon (e.g., coding region). These results are striking, as cells that are biallelically edited for two different large donor constructs at the same gene locus are usually very rare events when performing homologous recombination experiments in PSCs.

### EQUIVALENTS

It is to be understood that while the disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims.

We claim:

1. A method of editing the genome of a cell, the method comprising contacting the cell with:
a nuclease that causes a break within an endogenous coding sequence of an essential gene in the cell, wherein the essential gene encodes a gene product that is required for survival and/or proliferation of the cell, and
(ii) a donor template that comprises a knock-in cassette comprising an exogenous coding sequence for a gene product of interest in frame with and downstream (3′) of an exogenous coding sequence or partial coding sequence of the essential gene, wherein the knock-in cassette is integrated into the genome of the cell by homology-directed repair (HDR) of the break, resulting in a genome-edited cell that expresses:
(a) the gene product of interest, and
(b) the gene product encoded by the essential gene that is required for survival and/or proliferation of the cell, or a functional variant thereof.

2. The method of claim 1, wherein, if the knock-in cassette is not integrated into the genome of the cell by homology-directed repair (HDR) in the correct position or orientation, the cell no longer expresses the gene product encoded by the essential gene, or a functional variant thereof.

3. The method of claim 1 or 2, wherein the break is a double-strand break.

4. The method of any one of claims 1-3, wherein the break is located within the last 1000, 500, 400, 300, 200, 100, or 50 base pairs of the endogenous coding sequence of the essential gene.

5. The method of any one of claims 1-3, wherein the break is located within the last exon of the essential gene.

6. The method of any one of claims 1-5, wherein the nuclease is a CRISPR/Cas nuclease and the method further comprises contacting the cell with a guide molecule for the CRISPR/Cas nuclease.

7. The method of any one of claims 1-5, wherein the nuclease is a zinc finger nuclease (ZFN), a transcription activator-like effector nuclease (TALEN) or a meganuclease.

8. The method of any one of claims 1-7, wherein the donor template is a donor DNA template, optionally wherein the donor DNA template is double-stranded.

9. The method of claim 8, wherein the donor DNA template is a plasmid, optionally wherein the plasmid has not been linearized.

10. The method of any one of claims 1-9, wherein the donor template comprises homology arms on either side of the knock-in cassette.

11. The method of claim 10, wherein the homology arms correspond to sequences located on either side of the break in the genome of the cell.

12. The method of any one of claims 1-11, wherein the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product.

13. The method of claim 12, wherein the knock-in cassette comprises an IRES or 2A element located between the exogenous coding sequence or partial coding sequence of the essential gene and the exogenous coding sequence for the gene product of interest.

14. The method of claim 13, wherein the 2A element is a T2A element (EGRGSLLTCGDVEENPGP), a P2A element (ATNFSLLKQAGDVEENPGP), a E2A element (QCTNYALLKLAGDVESNPGP), or an F2A element (VKQTLNFDLLKLAGDVESNPGP).

15. The method of claim 13 or 14, wherein the knock-in cassette further comprises a sequence encoding a linker peptide upstream of the 2A element.

16. The method of claim 15, wherein the linker peptide comprises the amino acid sequence GSG.

17. The method of any one of claims 1-16, wherein the knock-in cassette comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

18. The method of any one of claims 1-17, wherein the exogenous partial coding sequence of the essential gene in the knock-in cassette encodes a C-terminal fragment of a protein encoded by the essential gene.

19. The method of claim 18, wherein the C-terminal fragment is less than 500, 250, 150, 125, 100, 75, 50, 25, 20, 15 or 10 amino acids in length.

20. The method of claim 18 or 19, wherein the C-terminal fragment includes an amino acid sequence that is encoded by a region of the endogenous coding sequence of the essential gene that spans the break.

21. The method of any one of claims 1-20, wherein the exogenous coding sequence or partial coding sequence of the essential gene in the knock-in cassette is less than 100% identical to the corresponding endogenous coding sequence of the essential gene of the cell.

22. The method of claim 21, wherein the exogenous coding sequence or partial coding sequence of the essential gene in the knock-in cassette has been codon optimized relative to the corresponding endogenous coding sequence of the essential gene of the cell to prevent further binding of the nuclease to the target site, to reduce the likelihood of recombination after integration of the knock-in cassette into the genome of the cell, and/or to increase expression of the gene product of the essential gene and/or the gene product of interest after integration of the knock-in cassette into the genome of the cell.

23. The method of any one of claims 1-22, wherein the essential gene is a housekeeping gene, e.g., a gene listed in Table 3.

24. The method of any one of claims 1-22, wherein the cell is an iPS cell or ES cell and the essential gene is involved in differentiation of iPS or ES cells or expansion of iPS- or ES-derived cells, e.g., a gene listed in Table 4.

25. The method of claim 24, wherein the iPS-derived cells are iPS-derived NK cells or iPS-derived T cells.

26. The method of any one of claims 1-25, wherein the donor template does not comprise a reporter gene, e.g., a fluorescent reporter gene or an antibiotic resistance gene.

27. The method of any one of claims 1-26, wherein the gene product of interest is a chimeric antigen receptor (CAR), a non-naturally occurring variant of FcγRIII (CD16), an interleukin (e.g., interleukin 15 (IL-15), interleukin 15 receptor (IL-15R) or a variant thereof, interleukin 12 (IL-12), interleukin-12 receptor (IL-12R) or a variant thereof), a human leukocyte antigen (e.g., human leukocyte antigen G (HLA-G), human leukocyte antigen E (HLA-E)), leukocyte surface antigen cluster of differentiation CD47 (CD47), or any combination of two or more thereof.

28. A genetically modified cell comprising a genome with an exogenous coding sequence for a gene product of interest in frame with and downstream (3′) of a coding sequence of an essential gene, wherein the essential gene encodes a gene product that is required for survival and/or proliferation of the cell.

29. An engineered cell comprising a genomic modification, wherein the genomic modification comprises an insertion of an exogenous knock-in cassette within an endogenous coding sequence of an essential gene in the cell's genome, wherein the essential gene encodes a gene product that is required for survival and/or proliferation of the cell, wherein the knock-in cassette comprises an exogenous coding sequence for a gene product of interest in frame with and downstream (3′) of an exogenous coding sequence or partial coding sequence encoding the gene product of the essential gene, or a functional variant thereof, and wherein the cell expresses the gene product of interest and the gene product encoded by the essential gene that is required for survival and/or proliferation of the cell, or a functional variant thereof, optionally wherein the gene product of interest and the gene product encoded by the essential gene are expressed from the endogenous promoter of the essential gene.

30. The cell of claim 28 or 29, wherein the cell's genome comprises a regulatory element that enables expression of the gene product encoded by the essential gene and the gene product of interest as separate gene products, optionally, wherein at least one of the gene products is a protein and the regulatory element enables expression of that protein separate from the other gene product.

31. The cell of claim 30, wherein the cell's genome comprises an IRES or 2A element located between the coding sequence of the essential gene and the exogenous coding sequence for the gene product of interest.

32. The cell of any one of claims 28-31, wherein the cell's genome comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

33. The cell of any one of claims 28-32, wherein the coding sequence of the essential gene is less than 100% identical to an endogenous coding sequence of the essential gene.

34. The cell of any one of claims 28-33, wherein the essential gene is a housekeeping gene, e.g., a gene listed in Table 3.

35. The cell of any one of claims 28-33, wherein the cell is an iPS cell or ES cell and the essential gene is involved in differentiation of iPS or ES cells or expansion of iPS- or ES-derived cells, e.g., a gene listed in Table 4.

36. The cell of claim 35, wherein the iPS-derived cells are iPS-derived NK cells or iPS-derived T cells.

37. The cell of any one of claims 28-36, wherein the cell's genome does not comprise a reporter gene, e.g., a fluorescent reporter gene or an antibiotic resistance gene.

38. The cell of any one of claims 28-37, wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.

39. The cell of any one of claims 28-38, for use as a medicament.

40. The cell of any one of claims 28-38, for use in the treatment of a disease, disorder, or condition, e.g., a cancer.

41. A cell, or population of cells, produced by the method of any one of claims 1-27 or progeny thereof.

42. A system for editing the genome of a cell, the system comprising the cell, a nuclease that causes a break within an endogenous coding sequence of an essential gene of the cell, wherein the essential gene encodes a gene product that is required for survival and/or proliferation of the cell, and a donor template that comprises a knock-in cassette comprising an exogenous coding sequence for a gene product of interest in frame with and downstream (3′) of an exogenous coding sequence or partial coding sequence of the essential gene.

43. The system of claim 42, wherein the break is a double-strand break.

44. The system of claim 42 or 43, wherein the break is located within the last 1000, 500, 400, 300, 200, 100 or 50 base pairs of the coding sequence of the essential gene.

45. The system of any one of claims 42-44, wherein the break is located within the last exon of the essential gene.

46. The system of any one of claims 42-45, wherein the nuclease is a CRISPR/Cas nuclease and the system further comprises a guide molecule for the CRISPR/Cas nuclease.

47. The system of any one of claims 42-45, wherein the nuclease is a zinc finger nuclease (ZFN), a transcription activator-like effector nuclease (TALEN) or a meganuclease.

48. The system of any one of claims 42-47, wherein the donor template is a donor DNA template, optionally wherein the donor DNA template is double-stranded.

49. The system of claim 48, wherein the donor DNA template is a plasmid, optionally wherein the plasmid has not been linearized.

50. The system of any one of claims 42-49, wherein the donor template comprises homology arms on either side of the knock-in cassette.

51. The system of claim 50, wherein the homology arms correspond to sequences located on either side of the break in the genome of the cell.

52. The system of any one of claims 42-51, wherein the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product.

53. The system of claim 52, wherein the knock-in cassette comprises an IRES or 2A element located between the exogenous coding sequence or partial coding sequence of the essential gene and the exogenous coding sequence for the gene product of interest.

54. The system of any one of claims 42-53, wherein the knock-in cassette comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

55. The system of any one of claims 42-54, wherein the exogenous partial coding sequence of the essential gene in the knock-in cassette encodes a C-terminal fragment of a protein encoded by the essential gene.

56. The system of claim 55, wherein the C-terminal fragment is less than 500, 250, 150, 125, 100, 75, 50, 25, 20, 15 or 10 amino acids in length.

57. The system of claim 55 or 56, wherein the C-terminal fragment includes an amino acid sequence that is encoded by a region of the coding sequence of the essential gene that spans the break.

58. The system of any one of claims 42-57, wherein the exogenous coding sequence or partial coding sequence of the essential gene in the knock-in cassette is less than 100% identical to the corresponding endogenous coding sequence of the essential gene of the cell.

59. The system of claim 58, wherein the exogenous coding sequence or partial coding sequence of the essential gene in the knock-in cassette has been codon optimized relative to the corresponding endogenous coding sequence of the essential gene of the cell to prevent further binding of a nuclease to the target site, to reduce the likelihood of recombination after integration of the knock-in cassette into the genome of the cell, or to increase expression of the gene product of the essential gene and/or the gene product of interest after integration of the knock-in cassette into the genome of the cell.

60. The system of claim 59, wherein the exogenous coding sequence or partial coding sequence of the essential gene in the knock-in cassette does not comprise a target site for the nuclease.

61. The system of any one of claims 42-60, wherein the essential gene is a housekeeping gene, e.g., a gene listed in Table 3.

62. The system of any one of claims 42-61, wherein the cell is an iPS cell or ES cell and the essential gene is involved in differentiation of iPS or ES cells or expansion of iPS- or ES-derived cells, e.g., a gene listed in Table 4.

63. The system of claim 62, wherein the iPS-derived cells are iPS-derived NK cells or iPS-derived T cells.

64. The system of any one of claims 42-63, wherein the donor DNA template does not comprise a reporter gene, e.g., a fluorescent reporter gene or an antibiotic resistance gene.

65. The system of any one of claims 42-64, wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.

66. A donor template comprising a knock-in cassette with an exogenous coding sequence for a gene product of interest in frame with and downstream (3′) of an exogenous coding sequence or partial coding sequence of an essential gene, wherein the essential gene encodes a gene product that is required for survival and/or proliferation of the cell.

67. The donor template of claim 66, for use in editing the genome of a cell by homology-directed repair (HDR)

68. The donor template of claim 66 or 67, wherein the donor template is a donor DNA template, optionally wherein the donor DNA template is double-stranded.

69. The donor template of claim 68, wherein the donor DNA template is a plasmid, optionally wherein the plasmid has not been linearized.

70. The donor template of any one of claims 66-69, wherein the donor template comprises homology arms on either side of the knock-in cassette.

71. The donor template of any one of claims 66-70, wherein the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product.

72. The donor template of claim 71, wherein the knock-in cassette comprises an 1RES or 2A element located between the exogenous coding sequence or partial coding sequence of the essential gene and the exogenous coding sequence for the gene product of interest.

73. The donor template of any one of claims 66-72, wherein the knock-in cassette comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

74. The donor template of any one of claims 66-73, wherein the exogenous partial coding sequence of the essential gene in the knock-in cassette encodes a C-terminal fragment of a protein encoded by the endogenous coding sequence of the essential gene.

75. The donor template of claim 74, wherein the C-terminal fragment is less than 500, 250, 150, 125, 100, 75, 50, 25, 20, 15 or 10 amino acids in length.

76. The donor template of any one of claims 66-75, wherein the exogenous coding sequence or partial coding sequence of the essential gene in the knock-in cassette is less than 100% identical to the corresponding endogenous coding sequence of the essential gene.

77. The donor template of claim 76, wherein the exogenous coding sequence or partial coding sequence of the essential gene in the knock-in cassette has been codon optimized relative to the corresponding endogenous coding sequence of the essential gene to prevent further binding of a nuclease to the target site, to reduce the likelihood of recombination after integration of the knock-in cassette into a genome of a cell, or to increase expression of the gene product of the essential gene and/or the gene product of interest after integration of the knock-in cassette into a genome of a cell.

78. The donor template of claim 77, wherein the exogenous coding sequence or partial coding sequence of the essential gene in the knock-in cassette does not comprise a target site for a nuclease.

79. The donor template of any one of claims 66-78, wherein the essential gene is a housekeeping gene, e.g., a gene listed in Table 3.

80. The donor template of any one of claims 66-79, wherein the cell is an iPS cell or ES cell and the essential gene is involved in differentiation of iPS or ES cells or expansion of iPS- or ES-derived cells, e.g., a gene listed in Table 4.

81. The donor template of any one of claims 66-80, wherein the donor template does not comprise a reporter gene, e.g., a fluorescent reporter gene or an antibiotic resistance gene.

82. The donor template of any one of claims 66-81, wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.

83. A method of generating genetically modified mammalian cells comprising a safety switch comprising:
providing at least one donor nucleic acid construct comprising a genetic payload comprising at least one necessary component of a safety switch
wherein said genetic payload is flanked by a first homologous region (HR) and a second HR, wherein the first and second HRs are essentially homologous to a first genomic region (GR) and a second GR, respectively, wherein the first GR and the second GR are adjacent to and flank a pre-determined genomic position in an exon of an essential gene in a mammalian cell,
providing a gene editing system containing a nuclease that is targeted to the pre-determined genomic position, and
adding the at least one donor nucleic acid construct and the gene editing system into a population of mammalian cells wherein a plurality of the mammalian cells incorporate the genetic payload at the pre-determined genomic position,
wherein a disruption to the essential gene sequence caused by the nuclease is restored upon integration of the HRs and genetic payload.

84. The method of claim 83 wherein each donor nucleic acid construct comprises at least one necessary component of the safety switch.

85. The method of any of claims 83 or 84, wherein each donor nucleic acid construct comprises all of the necessary components of a safety switch.

86. The method of any one of claims 83-85 wherein a combination of the donor nucleic acid constructs contain all of the necessary components of a functional safety switch.

87. The method of any one of claims 83-86 wherein the necessary components of the safety switch dimerize to produce a functional suicide switch.

88. The method of any one of claims 83-87 wherein the genetic payload from a first donor nucleic acid construct is incorporated into a first allele of the essential gene and the genetic payload from a second donor nucleic acid construct is incorporated into a second allele of the essential gene.

89. The method of any one of claims 83-88 wherein one or more of the necessary components of the safety switch are incorporated into a first allele of the essential gene and the rest of the necessary components of the safety switch are incorporated into the second allele of the essential gene.

90. The method of any one of claims 83-89 wherein activation of the safety switch is triggered by a cellular event, an environmental event or a chemical agent.

91. The method of any of claims 83-90 wherein activation of the safety switch induces apoptosis.

92. The method of any one of claims 83-91 wherein activation of the safety switch inhibits growth of cells that have incorporated all of the necessary components of the safety switch.

93. A population of cells made by the method of any one of claims 83-92.

94. The population of cells of claim 93, wherein the cells are pluripotent stem cells (PSCs).

95. The population of cells of claim 93, wherein the cells are induced pluripotent stem cells (iPSCs).

96. A cell from the population of cells of any one of claims 93-95 wherein the cell is differentiated into a differentiated cell.

97. The differentiated cell of claim 96, wherein the differentiated cell is selected from:
a cell in the immune system, optionally selected from a T cell, a T cell expressing a chimeric antigen receptor (CAR), a suppressive T cell, a myeloid cell, a dendritic cell, and a macrophage;
a cell in the nervous system, optionally selected from a dopaminergic neuron, a microglia cell, an oligodendrocyte, an astrocyte, a cortical neuron, a spinal or oculomotor neuron, an enteric neuron, a Placode-derived cell, a Schwann cell, and a trigeminal or sensory neuron;
a cell in the ocular system, optionally selected from a retinal pigment epithelial cell, a photoreceptor cone cell, a photoreceptor rod cell, a bipolar cell, and a ganglion cell;
a cell in the cardiovascular system, optionally selected from a cardiomyocyte, an endothelial cell, and a nodal cell; or
a cell in the metabolic system, optionally selected from a hepatocyte, a cholangiocyte, and a pancreatic beta cell.

98. A method of increasing the percentage of cells in the population of cells of claim 93-97 that incorporate the genetic payload at the pre-determined genomic position comprising:
creating a first population of mammalian cells comprising cells of any of claims 93-97 by providing at least one donor nucleic acid construct comprising a specific genetic payload flanked by a first homologous region (HR) and a second HR, wherein the first and second HRs are essentially homologous to a first genomic region (GR) and a second GR, respectively, wherein the first GR and the second GR are adjacent to and flank a pre-determined genomic position in an exon of an essential gene in a mammalian cell,
providing a gene editing system containing a nuclease that is targeted to the pre-determined genomic position,
providing the at least one donor nucleic acid construct and the gene editing system into the first population of mammalian cells,
culturing the first population of mammalian cells, and
identifying the percentage of surviving cells that comprise the specific genetic payload,
creating a second population of mammalian cells by expanding the surviving cells from the first population of mammalian cells by providing to the surviving cells from the first population of mammalian cells, a gene editing system containing a nuclease that is targeted to the pre-determined genomic position;
optionally reintroducing the at least one donor construct;
culturing the second population of mammalian cells; and
identifying the percentage of surviving cells that comprise the specific exogenous genetic payload,
wherein the percentage of surviving cells from the second population of mammalian cells that comprise the specific exogenous genetic payload is higher than the percentage of surviving cells from the first population of mammalian cells that comprise the specific exogenous genetic payload.

99. The method of claim 98 wherein a plurality of the surviving cells from the first population of mammalian cells that do not comprise the specific genetic payloads are killed during the creation of the second population of mammalian cells.

100. The method of any one of claim 98 or 99 wherein a plurality of surviving cells from the first population of mammalian cells that do not comprise the specific genetic payloads incorporate the specific genetic payloads during the creation of the second population of mammalian cells.

101. The method of any one of claims 98-100 wherein the percentage of surviving cells from the second population of mammalian cells that comprise the specific genetic payloads is at least three times larger than the percentage of surviving cells from the first population of mammalian cells that comprise the specific genetic payloads.

102. The method of any one of the preceding claims 98-101 wherein the percentage of surviving cells from the second population of mammalian cells that do not comprise the specific genetic payloads is at least five (5) times lower than the percentage of surviving cells from the first population of mammalian cells that do not comprise the specific genetic payloads.

103. The method of any one of claims 98-102 wherein at least one of the donor nucleic acid constructs has a different genetic payload than at least one other donor nucleic acid constructs, and at least a plurality of the second population of mammalian cells incorporate each of the different genetic payloads.

104. The method of any one of claims 98-103 wherein the at least one of the HR regions contains at least one mutation that prevents the cutting of the genetic payload at the nuclease cutting site.

105. The method of claims 98-104 wherein identifying the percentage of surviving cells is accomplished using flow cytometry.

106. An engineered iPSC comprising a genomic modification, wherein the genomic modification comprises an insertion of an exogenous knock-in cassette within an endogenous coding sequence of a GAPDH gene in the iPSC's genome, wherein the knock-in cassette comprises an exogenous coding sequence for a safety switch in frame with and downstream (3′) of an exogenous coding sequence or partial coding sequence encoding GAPDH, or a functional variant thereof, and wherein the iPSC expresses the gene product of interest and GAPDH, or a functional variant thereof, optionally wherein the gene product of interest and the GAPDH are expressed from the endogenous promoter of the GAPDH gene.

107. The iPSC of claim 106, wherein the iPSC's genome comprises a regulatory element that enables expression of GAPDH and the gene product of interest as separate gene products, optionally, wherein at least one of the gene products is a protein and the regulatory element enables expression of that protein separate from the other gene product.

108. The iPSC of claim 107, wherein the iPSC's genome comprises an IRES or 2A element located between the coding sequence of the GAPDH gene and the exogenous coding sequence for the gene product of interest.

109. The iPSC of any one of claims 106-108, wherein the iPSC's genome comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

110. The iPSC of any one of claims 106-109, wherein the coding sequence of the GAPDH gene is less than 100% identical to an endogenous coding sequence of the GAPDH gene.

111. The iPSC of any one of claims 106-110, wherein the iPSC's genome does not comprise a reporter gene, e.g., a fluorescent reporter gene or an antibiotic resistance gene.

112. The iPSC of any one of claims 106-111, for use as a medicament.

113. The iPSC of any one of claims 106-112, for use in the treatment of a disease, disorder, or condition, e.g., a cancer.

114. A system for editing the genome of an iPSC in a population of iPSCs, the system comprising the population of iPSC, a nuclease that causes a break within an endogenous coding sequence of a GAPDH gene of the iPSC, and a donor template that comprises a knock-in cassette comprising a safety switch in frame with and downstream (3′) of an exogenous coding sequence or partial coding sequence of the GAPDH gene.

115. The system of claim 114, wherein the break is a double-strand break.

116. The system of any one of claims 114-115, wherein the break is located within the last exon of the GAPDH gene.

117. The system of any one of claims 114-116, wherein the nuclease is a CRISPR/Cas nuclease and the system further comprises a guide molecule for the CRISPR/Cas nuclease.

118. The system of any one of claims 114-116, wherein the nuclease is a zinc finger nuclease (ZFN), a transcription activator-like effector nuclease (TALEN) or a meganuclease.

119. The system of any one of claims 114-118, wherein the knock-in cassette comprises a regulatory element that enables expression of GAPDH and the gene product of interest as separate gene products, optionally, wherein at least one of the gene products is a protein and the regulatory element enables expression of that protein separate from the other gene product.

120. The system of claim 119, wherein the knock-in cassette comprises an IRES or 2A element located between the exogenous coding sequence or partial coding sequence of the GAPDH gene and the exogenous coding sequence for the gene product of interest.

121. The system of any one of claims 114-120, wherein the knock-in cassette comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

122. The system of any one of claims 114-121, wherein the exogenous coding sequence or partial coding sequence of the GAPDH gene in the knock-in cassette is less than 100% identical to the corresponding endogenous coding sequence of the GAPDH gene of the iPSC.

123. The system of claim 122, wherein the exogenous coding sequence or partial coding sequence of the GAPDH gene in the knock-in cassette has been codon optimized relative to the corresponding endogenous coding sequence of the GAPDH gene of the iPSC to remove a target site of the DNA nuclease, and/or to reduce the likelihood of homologous recombination after integration of the knock-in cassette into the genome of the iPSC.

124. The system of claim 123, wherein the exogenous coding sequence or partial coding sequence of the GAPDH gene in the knock-in cassette does not comprise a target site for the nuclease.

125. A method of increasing the percentage of genetically modified mammalian cells with a desired genetic payload within a population of mammalian cells comprising:
creating a first population of mammalian cells by providing at least one donor nucleic acid construct comprising a specific genetic payload flanked by a first homologous region (HR) and a second HR, wherein the first and second HRs are essentially homologous to a first genomic region (GR) and a second GR, respectively, wherein the first GR and the second GR are adjacent to and flank a pre-determined genomic position in an exon of an essential gene in a mammalian cell,
providing a gene editing system containing a nuclease that is targeted to the pre-determined genomic position,
providing the at least one donor nucleic acid construct and the gene editing system into the first population of mammalian cells,
culturing the first population of mammalian cells, and
identifying the percentage of surviving cells that comprise the specific genetic payload,
creating a second population of mammalian cells by providing to the surviving cells from the first population of mammalian cells, a gene editing system containing a nuclease that is targeted to the pre-determined genomic position;
optionally reintroducing the at least one donor construct;
culturing the second population of mammalian cells; and
identifying the percentage of surviving cells that comprise the specific exogenous genetic payload,
wherein the percentage of surviving cells from the second population of mammalian cells that comprise the specific exogenous genetic payload is higher than the percentage of surviving cells from the first population of mammalian cells that comprise the specific exogenous genetic payload.

126. The method of claim 125 wherein a plurality of the surviving cells from the first population of mammalian cells that do not comprise the specific genetic payloads are killed during the creation of the second population of mammalian cells.

127. The method any one of claims 125-126 wherein a plurality of surviving cells from the first population of mammalian cells that do not comprise the specific genetic payloads incorporate the specific genetic payloads during the creation of the second population of mammalian cells.

128. The method of any one of claims 125-127 wherein the percentage of surviving cells from the second population of mammalian cells that comprise the specific genetic payloads is at least three (3) times larger than the percentage of surviving cells from the first population of mammalian cells that comprise the specific genetic payloads.

129. The method of any one of claims 125-128 wherein at least one of the donor nucleic acid constructs has a different genetic payload than at least one other donor nucleic acid constructs, and at least a plurality of the second population of mammalian cells incorporate each of the different genetic payloads.

130. The method of any one of claims 125-129 wherein the percentage of surviving cells from the second population of mammalian cells that do not comprise the specific genetic payloads is at least five (5) times lower than the percentage of surviving cells from the first population of mammalian cells that do not comprise the specific genetic payloads.

131. The method of any one of claims 125-130 wherein the at least one of the HR regions contains at least one mutation that prevents the cutting of the genetic payload at the nuclease cutting site.

132. The method of any one of claims 125-131 wherein identifying the percentage of surviving cells is accomplished using flow cytometry.

133. A population of cells made by the method of any one of claims 125-132.

134. The population of cells of claim 133, wherein the cells are pluripotent stem cells (PSCs).

135. The population of cells of claim 133, wherein the cells are induced pluripotent stem cells (iPSCs).

136. A cell from the population of cells of any one of claims 133-135 wherein the cell is differentiated into a differentiated cell.

137. The differentiated cell of claim 136, wherein the differentiated cell is selected from:
a cell in the immune system, optionally selected from a T cell, a T cell expressing a chimeric antigen receptor (CAR), a suppressive T cell, a myeloid cell, a dendritic cell, and a macrophage;
a cell in the nervous system, optionally selected from a dopaminergic neuron, a microglia cell, an oligodendrocyte, an astrocyte, a cortical neuron, a spinal or oculomotor neuron, an enteric neuron, a Placode-derived cell, a Schwann cell, and a trigeminal or sensory neuron;
a cell in the ocular system, optionally selected from a retinal pigment epithelial cell, a photoreceptor cone cell, a photoreceptor rod cell, a bipolar cell, and a ganglion cell;
a cell in the cardiovascular system, optionally selected from a cardiomyocyte, an endothelial cell, and a nodal cell; or
a cell in the metabolic system, optionally selected from a hepatocyte, a cholangiocyte, and a pancreatic beta cell.

138. An iPSC of claim 135, wherein the iPSC's genome comprises a regulatory element that enables expression of GAPDH and the gene product of interest as separate gene products, optionally, wherein at least one of the gene products is a protein and the regulatory element enables expression of that protein separate from the other gene product.

139. The iPSC of claim 138, wherein the iPSC's genome comprises an IRES or 2A element located between the coding sequence of the GAPDH gene and the exogenous coding sequence for the gene product of interest.

140. The iPSC of any one of claims 138-139, wherein the iPSC's genome comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

141. The iPSC of any one of claims 138-140, wherein the coding sequence of the GAPDH gene is less than 100% identical to an endogenous coding sequence of the GAPDH gene.

142. The iPSC of any one of claims 138-141, wherein the iPSC's genome does not comprise a reporter gene, e.g., a fluorescent reporter gene or an antibiotic resistance gene.

143. The iPSC of any one of claims 138-142, for use as a medicament.

144. The iPSC of any one of claims 138-143, for use in the treatment of a disease, disorder, or condition, e.g., a cancer.

145. A method of editing the genome of a pluripotent stem cell or an iPS cell, the method comprising contacting the cell with:
a nuclease that causes a break within an endogenous coding sequence of an essential gene in the cell, wherein the essential gene encodes a gene product that is required for survival and/or proliferation of the cell, and
(ii) a donor template that comprises a knock-in cassette comprising an exogenous coding sequence for a gene product of interest in frame with and downstream (3′) of an exogenous coding sequence or partial coding sequence of the essential gene, wherein the knock-in cassette is integrated into the genome of the cell by homology-directed repair (HDR) of the break, resulting in a genome-edited cell that expresses:
(a) the gene product of interest, and
(b) the gene product encoded by the essential gene that is required for survival and/or proliferation of the cell, or a functional variant thereof.

146. The method of claim 145, wherein, if the knock-in cassette is not integrated into the genome of the cell by homology-directed repair (HDR) in the correct position or orientation, the cell no longer expresses the gene product encoded by the essential gene, or a functional variant thereof.

147. The method of claim 145 or 146, wherein the break is a double-strand break.

148. The method of any one of claims 145-147, wherein the break is located within the last 1000, 500, 400, 300, 200, 100, or 50 base pairs of the endogenous coding sequence of the essential gene.

149. The method of any one of claims 145-147, wherein the break is located within the last exon of the essential gene.

150. The method of any one of claims 145-149, wherein the nuclease is a CRISPR/Cas nuclease and the method further comprises contacting the cell with a guide molecule for the CRISPR/Cas nuclease.

151. The method of any one of claims 145-149, wherein the nuclease is a zinc finger nuclease (ZFN), a transcription activator-like effector nuclease (TALEN) or a meganuclease.

152. The method of any one of claims 145-151, wherein the donor template is a donor DNA template, optionally wherein the donor DNA template is double-stranded.

153. The method of claim 152, wherein the donor DNA template is a plasmid, optionally wherein the plasmid has not been linearized.

154. The method of any one of claims 145-153, wherein the donor template comprises homology arms on either side of the knock-in cassette.

155. The method of claim 154, wherein the homology arms correspond to sequences located on either side of the break in the genome of the cell.

156. The method of any one of claims 145-155, wherein the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product.

157. The method of claim 156, wherein the knock-in cassette comprises an IRES or 2A element located between the exogenous coding sequence or partial coding sequence of the essential gene and the exogenous coding sequence for the gene product of interest.

158. The method of claim 157, wherein the 2A element is a T2A element (EGRGSLLTCGDVEENPGP), a P2A element (ATNFSLLKQAGDVEENPGP), a E2A element (QCTNYALLKLAGDVESNPGP), or an F2A element (VKQTLNFDLLKLAGDVESNPGP).

159. The method of claim 157 or 158, wherein the knock-in cassette further comprises a sequence encoding a linker peptide upstream of the 2A element.

160. The method of claim 159, wherein the linker peptide comprises the amino acid sequence GSG.

161. The method of any one of claims 145-160, wherein the knock-in cassette comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

162. The method of any one of claims 145-161, wherein the exogenous partial coding sequence of the essential gene in the knock-in cassette encodes a C-terminal fragment of a protein encoded by the essential gene.

163. The method of claim 162, wherein the C-terminal fragment is less than 500, 250, 150, 125, 100, 75, 50, 25, 20, 15 or 10 amino acids in length.

164. The method of claim 162 or 163, wherein the C-terminal fragment includes an amino acid sequence that is encoded by a region of the endogenous coding sequence of the essential gene that spans the break.

165. The method of any one of claims 145-164, wherein the exogenous coding sequence or partial coding sequence of the essential gene in the knock-in cassette is less than 100% identical to the corresponding endogenous coding sequence of the essential gene of the cell.

166. The method of claim 165, wherein the exogenous coding sequence or partial coding sequence of the essential gene in the knock-in cassette has been codon optimized relative to the corresponding endogenous coding sequence of the essential gene of the cell to prevent further binding of the nuclease to the target site, to reduce the likelihood of recombination after integration of the knock-in cassette into the genome of the cell, and/or to increase expression of the gene product of the essential gene and/or the gene product of interest after integration of the knock-in cassette into the genome of the cell.

167. The method of any one of claims 145-166, wherein the essential gene is a housekeeping gene, e.g., a gene listed in Table 3.

168. The method of any one of claims 145-166, wherein the cell is an iPS cell or ES cell and the essential gene is involved in differentiation of iPS or ES cells or expansion of iPS- or ES-derived cells, e.g., a gene listed in Table 4.

169. The method of claim 168, wherein the iPS-derived cells are iPS-derived NK cells or iPS-derived T cells.

170. The method of any one of claims 145-169, wherein the donor template does not comprise a reporter gene, e.g., a fluorescent reporter gene or an antibiotic resistance gene.

171. The method of any one of claims 145-170, wherein the gene product of interest is a chimeric antigen receptor (CAR), a non-naturally occurring variant of FcγRIII (CD16), an interleukin (e.g., interleukin 15 (IL-15), interleukin 15 receptor (IL-15R) or a variant thereof, interleukin 12 (IL-12), interleukin-12 receptor (IL-12R) or a variant thereof), a human leukocyte antigen (e.g., human leukocyte antigen G (HLA-G), human leukocyte antigen E (HLA-E)), leukocyte surface antigen cluster of differentiation CD47 (CD47), or any combination of two or more thereof.

172. A genetically modified pluripotent stem cell or iPS cell comprising a genome with an exogenous coding sequence for a gene product of interest in frame with and downstream (3′) of a coding sequence of an essential gene, wherein the essential gene encodes a gene product that is required for survival and/or proliferation of the cell.

173. An engineered pluripotent stem cell or iPS cell comprising a genomic modification, wherein the genomic modification comprises an insertion of an exogenous knock-in cassette within an endogenous coding sequence of an essential gene in the cell's genome, wherein the essential gene encodes a gene product that is required for survival and/or proliferation of the cell, wherein the knock-in cassette comprises an exogenous coding sequence for a gene product of interest in frame with and downstream (3′) of an exogenous coding sequence or partial coding sequence encoding the gene product of the essential gene, or a functional variant thereof, and wherein the cell expresses the gene product of interest and the gene product encoded by the essential gene that is required for survival and/or proliferation of the cell, or a functional variant thereof, optionally wherein the gene product of interest and the gene product encoded by the essential gene are expressed from the endogenous promoter of the essential gene.

174. The cell of claim 172 or 173, wherein the cell's genome comprises a regulatory element that enables expression of the gene product encoded by the essential gene and the gene product of interest as separate gene products, optionally, wherein at least one of the gene products is a protein and the regulatory element enables expression of that protein separate from the other gene product.

175. The cell of claim 174, wherein the cell's genome comprises an IRES or 2A element located between the coding sequence of the essential gene and the exogenous coding sequence for the gene product of interest.

176. The cell of any one of claims 172-175, wherein the cell's genome comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

177. The cell of any one of claims 172-176, wherein the coding sequence of the essential gene is less than 100% identical to an endogenous coding sequence of the essential gene.

178. The cell of any one of claims 172-177, wherein the essential gene is a housekeeping gene, e.g., a gene listed in Table 3.

179. The cell of any one of claims 172-177, wherein the cell is an iPS cell or ES cell and the essential gene is involved in differentiation of iPS or ES cells or expansion of iPS- or ES-derived cells, e.g., a gene listed in Table 4.

180. The cell of claim 179, wherein the iPS-derived cells are iPS-derived NK cells or iPS-derived T cells.

181. The cell of any one of claims 172-180, wherein the cell's genome does not comprise a reporter gene, e.g., a fluorescent reporter gene or an antibiotic resistance gene.

182. The cell of any one of claims 172-181, wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.

183. The cell of any one of claims 172-182, for use as a medicament.

184. The cell of any one of claims 172-182, for use in the treatment of a disease, disorder, or condition, e.g., a cancer.

185. A cell, or population of cells, produced by the method of any one of claims 145-171 or progeny thereof.

186. A system for editing the genome of a pluripotent stem cell or an iPS cell, the system comprising the cell, a nuclease that causes a break within an endogenous coding sequence of an essential gene of the cell, wherein the essential gene encodes a gene product that is required for survival and/or proliferation of the cell, and a donor template that comprises a knock-in cassette comprising an exogenous coding sequence for a gene product of interest in frame with and downstream (3′) of an exogenous coding sequence or partial coding sequence of the essential gene.

187. The system of claim 186, wherein the break is a double-strand break.

188. The system of claim 186 or 187, wherein the break is located within the last 1000, 500, 400, 300, 200, 100 or 50 base pairs of the coding sequence of the essential gene.

189. The system of any one of claims 186-188, wherein the break is located within the last exon of the essential gene.

190. The system of any one of claims 186-189, wherein the nuclease is a CRISPR/Cas nuclease and the system further comprises a guide molecule for the CRISPR/Cas nuclease.

191. The system of any one of claims 186-189, wherein the nuclease is a zinc finger nuclease (ZFN), a transcription activator-like effector nuclease (TALEN) or a meganuclease.

192. The system of any one of claims 186-191, wherein the donor template is a donor DNA template, optionally wherein the donor DNA template is double-stranded.

193. The system of claim 192, wherein the donor DNA template is a plasmid, optionally wherein the plasmid has not been linearized.

194. The system of any one of claims 186-193, wherein the donor template comprises homology arms on either side of the knock-in cassette.

195. The system of claim 194, wherein the homology arms correspond to sequences located on either side of the break in the genome of the cell.

196. The system of any one of claims 186-195, wherein the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product.

197. The system of claim 196, wherein the knock-in cassette comprises an IRES or 2A element located between the exogenous coding sequence or partial coding sequence of the essential gene and the exogenous coding sequence for the gene product of interest.

198. The system of any one of claims 186-197, wherein the knock-in cassette comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

199. The system of any one of claims 186-198, wherein the exogenous partial coding sequence of the essential gene in the knock-in cassette encodes a C-terminal fragment of a protein encoded by the essential gene.

200. The system of claim 199, wherein the C-terminal fragment is less than 500, 250, 150, 125, 100, 75, 50, 25, 20, 15 or 10 amino acids in length.

201. The system of claim 199 or 200, wherein the C-terminal fragment includes an amino acid sequence that is encoded by a region of the coding sequence of the essential gene that spans the break.

202. The system of any one of claims 186-201, wherein the exogenous coding sequence or partial coding sequence of the essential gene in the knock-in cassette is less than 100% identical to the corresponding endogenous coding sequence of the essential gene of the cell.

203. The system of claim 202, wherein the exogenous coding sequence or partial coding sequence of the essential gene in the knock-in cassette has been codon optimized relative to the corresponding endogenous coding sequence of the essential gene of the cell to prevent further binding of a nuclease to the target site, to reduce the likelihood of recombination after integration of the knock-in cassette into the genome of the cell, or to increase expression of the gene product of the essential gene and/or the gene product of interest after integration of the knock-in cassette into the genome of the cell.

204. The system of claim 203, wherein the exogenous coding sequence or partial coding sequence of the essential gene in the knock-in cassette does not comprise a target site for the nuclease.

205. The system of any one of claims 186-204, wherein the essential gene is a housekeeping gene, e.g., a gene listed in Table 3.

206. The system of any one of claims 186-205, wherein the cell is an iPS cell or ES cell and the essential gene is involved in differentiation of iPS or ES cells or expansion of iPS- or ES-derived cells, e.g., a gene listed in Table 4.

207. The system of claim 206, wherein the iPS-derived cells are iPS-derived NK cells or iPS-derived T cells.

208. The system of any one of claims 186-207, wherein the donor DNA template does not comprise a reporter gene, e.g., a fluorescent reporter gene or an antibiotic resistance gene.

209. The system of any one of claims 186-208, wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.

210. A donor template for use in editing the genome of a pluripotent stem cell or an iPS cell, the donor template comprising a knock-in cassette with an exogenous coding sequence for a gene product of interest in frame with and downstream (3′) of an exogenous coding sequence or partial coding sequence of an essential gene, wherein the essential gene encodes a gene product that is required for survival and/or proliferation of a pluripotent stem cell or an iPS cell.

211. The donor template of claim 210, wherein the knock-in cassette is integrated into the genome of the cell by homology-directed repair (HDR).

212. The donor template of claim 210 or 211, wherein the donor template is a donor DNA template, optionally wherein the donor DNA template is double-stranded.

213. The donor template of any one of claims 210-212, wherein the donor DNA template is a plasmid, optionally wherein the plasmid has not been linearized.

214. The donor template of any one of claims 210-213, wherein the donor template comprises homology arms on either side of the knock-in cassette.

215. The donor template of any one of claims 210-214, wherein the knock-in cassette comprises a regulatory element that enables expression of the gene product encoded by the essential gene and the gene product of interest as separate gene products, optionally, wherein at least one of the gene products is a protein and the regulatory element enables expression of that protein separate from the other gene product.

216. The donor template of claim 215, wherein the knock-in cassette comprises an IRES or 2A element located between the exogenous coding sequence or partial coding sequence of the essential gene and the exogenous coding sequence for the gene product of interest.

217. The donor template of any one of claims 210-216, wherein the knock-in cassette comprises a polyadenylation sequence, and optionally a 3′ UTR sequence, downstream of the exogenous coding sequence for the gene product of interest, wherein, if a 3′UTR sequence is present, the 3′UTR sequence is positioned 3′ of the exogenous coding sequence and 5′ of the polyadenylation sequence.

218. The donor template of any one of claims 210-217, wherein the exogenous partial coding sequence of the essential gene in the knock-in cassette encodes a C-terminal fragment of a protein encoded by the endogenous coding sequence of the essential gene.

219. The donor template of claim 218, wherein the C-terminal fragment is less than 500, 250, 150, 125, 100, 75, 50, 25, 20, 15 or 10 amino acids in length.

220. The donor template of any one of claims 210-219, wherein the exogenous coding sequence or partial coding sequence of the essential gene in the knock-in cassette is less than 100% identical to the corresponding endogenous coding sequence of the essential gene.

221. The donor template of claim 220, wherein the exogenous coding sequence or partial coding sequence of the essential gene in the knock-in cassette has been codon optimized relative to the corresponding endogenous coding sequence of the essential gene to prevent further binding of a nuclease to the target site, to reduce the likelihood of recombination after integration of the knock-in cassette into a genome of a cell, or to increase expression of the gene product of the essential gene and/or the gene product of interest after integration of the knock-in cassette into a genome of a cell.

222. The donor template of claim 221, wherein the exogenous coding sequence or partial coding sequence of the essential gene in the knock-in cassette does not comprise a target site for a nuclease.

223. The donor template of any one of claims 210-222, wherein the essential gene is a housekeeping gene, e.g., a gene listed in Table 3.

224. The donor template of any one of claims 210-223, wherein the cell is an iPS cell or ES cell and the essential gene is involved in differentiation of iPS or ES cells or expansion of iPS- or ES-derived cells, e.g., a gene listed in Table 4.

225. The donor template of any one of claims 210-224, wherein the donor template does not comprise a reporter gene, e.g., a fluorescent reporter gene or an antibiotic resistance gene.

226. The donor template of any one of claims 210-225, wherein the gene product of interest is a chimeric antigen receptor (CAR), a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof.
